(19) United States
(12) Patent Application Publication
(10) Pub. No.: US 2024/0158418 A1
(43) Pub. Date: May 16, 2024

(54) EGFR Degraders to Treat Cancer Metastasis to the Brain or CNS

(71) Applicant: C4 Therapeutics, Inc., Watertown, MA (US)

(72) Inventors: Christopher G. Nasveschuk, Stoneham, MA (US); Martin Duplessis, Somerville, MA (US); Jae Young Ahn, Somerville, MA (US); Alexander W. Hird, Belmont, MA (US); Ryan E. Michael, Erie, CO (US); Kiel Lazarski, Boston, MA (US); Yanke Liang, Belmont, MA (US); Georg Jaeschke, Basel (CH); Antonio Ricci, Beil-Benken (CH); Annick Goergler, Colmar (FR); Daniel Rueher, Raedersdorf (FR)

(73) Assignee: C4 Therapeutics, Inc., Watertown, MA (US)

(21) Appl. No.: 18/516,589

(22) Filed: Nov. 21, 2023

Related U.S. Application Data
(63) Continuation of application No. PCT/US2022/031193, filed on May 26, 2022.
(60) Provisional application No. 63/270,488, filed on Oct. 21, 2021.
(60) Provisional application No. 63/193,574, filed on May 26, 2021.

(51) Int. Cl. C07D 519/00; A61P 35/04

(52) U.S. Cl. CPC C07D 519/00; A61P 35/04

(57) ABSTRACT

The invention provides for the treatment of mutant epidermal growth factor receptor (EGFR) mediated cancer that has metastasized to the brain or other area of the central nervous system with a compound that degrades a mutant form of EGFR via the ubiquitination of the EGFR protein and subsequent proteasomal degradation. The invention also provides advantageous drug combinations for the treatment of such cancer that include a compound herein that degrades a mutant form of EGFR in combination with a second anti-cancer agent.

### CROSS-REFERENCE TO RELATED APPLICATIONS

This application is a continuation of International Patent Application No. PCT/US2022/031193, filed in the U.S. Receiving Office on May 26, 2022, which claims the benefit of U.S. Provisional Application 63/193,574, filed May 26, 2021, and U.S. Provisional Application 63/270,488, filed Oct. 21, 2021. The entirety of each of these applications is incorporated herein by reference for all purposes.

### FIELD OF THE INVENTION

The invention provides for the treatment of mutant epidermal growth factor receptor (EGFR) mediated cancer that has metastasized to the brain or other area of the central nervous system with a compound that degrades a mutant form of EGFR via the ubiquitination of the EGFR protein and subsequent proteasomal degradation. The invention also provides advantageous drug combinations for the treatment of such cancer that include a compound herein that degrades a mutant form of EGFR in combination with a second anti-cancer agent.

### BACKGROUND OF THE INVENTION

The HER family receptor tyrosine kinases are mediators of cell growth, differentiation, and survival. The receptor family includes four distinct members, i.e. epidermal growth factor receptor (EGFR, ErbB1, or HER1), HER2 (ErbB2), HER3 (ErbB3) and HER4 (ErbB4). Upon ligand binding, the receptors form homo and heterodimers and subsequent activation of the intrinsic tyrosine kinase activity leads to receptor auto-phosphorylation and the activation of downstream signaling molecules (Yarden, Y., Sliwkowski, M X. Untangling the ErbB signaling network. Nature Review Mol Cell Biol. 2001 February; 2(2): 127-37). These signaling molecules promote cell growth and proliferation. Deregulation of EGFR by overexpression or mutation has been implicated in many types of human cancer including colorectal, pancreatic, gliomas, head and neck and lung cancer, in particular non-small cell lung cancer (NSCLC). Several EGFR targeting agents have been developed over the years (Ciardiello, F., and Tortora, G. (2008). EGFR antagonists in cancer treatment. The New England Journal of Medicine 358, 1160-1174). Erlotinib (TARCEVA®) gefitinib (IRESSA®) are first generation reversible inhibitors of the EGFR tyrosine kinase that are approved in numerous countries for the treatment of recurrent NSCLC. Osimertinib (TAGRISSO®) is an irreversible inhibitor of the EGFR tyrosine kinase and is approved in numerous countries for the first line treatment of NSCLC (Soina et al., (2018) The New England Journal of Medicine 378, 113-125).

The most common somatic mutations of EGFR are exon 19 deletions and exon 21 amino acid substitutions. The most prevalent exon 19 deletions are delta 746-750 and the prevalent exon 21 amino acid substitution is L858R (Sharma S V, Bell D W, Settleman J, Haber D A. Epidermal growth factor receptor mutations in lung cancer. Nat Rev Cancer. 2007 March; 7(3): 169-81).

Treatment resistance arises frequently after first generation EGFR inhibitor treatment, often due to the secondary T790M mutation within the ATP binding site of the receptor. Osimertinib, a mutant-selective irreversible inhibitor, is highly active against the T790M mutant, but its efficacy can be compromised by acquired mutation of C797S, which is the cysteine residue with which osimertinib form a key covalent bond (Thress, K. S. et al. Acquired EGFR C797S mutation mediates resistance to AZD9291 in non-small cell lung cancer harboring EGFR T790M. Nat. Med. 21, 560-562 (2015)). C797S mutation was further reported by Wang et al. to be a major mechanism for resistance to T790M-targeting EGFR inhibitors (Wang et al. EGFR C797S mutation mediates resistance to third-generation inhibitors in T790M-positive non-small cell lung cancer, J Hematol Oncol. 2016; 9: 59). Additional mutations that cause resistance to osimertinib are described by Yang et al., for example L718Q (Yang et al, Investigating Novel Resistance Mechanisms to Third-Generation EGFR Tyrosine Kinase Inhibitor Osimertinib in Non-Small Cell Lung Cancer Patients, Clinical Cancer Research, DOI: 10.1158/1078-0432.CCR-17-2310). Additional mutations targeting strategies are also known including targeting EGFR^{L858R-T790M} and EGFR^{L858R-T790M-C797S} resistance mutations in NSCLC treatment (Lu et al. Targeting EGFR^{L858R-T790M} and EGFR^{L858R-T790M-C797S} resistance mutations in NSCLC: Current developments in medicinal chemistry, Med Res Rev 2018; 1-32).

Additional examples of EGFR inhibitors, in particular selective inhibitors of T790M containing EGFR mutants, have also been described including those in WO2014081718, WO2014210354, WO2018/115218, WO2018220149, WO2020002487, and ZHOU et al., “Novel mutant-selective EGFR kinase inhibitors against EGFR T790M”, NATURE, (20091224), vol. 462, no. 7276, doi:10.1038/nature08622, ISSN 0028-0836, pages 1070-1074.

All approved EGFR inhibitors target the ATP binding site of the kinase. As secondary mutations that cause resistance to ATP-competitive EGFR inhibitors are located in the ATP binding site, there is a need for new therapeutic agents that work differently to overcome resistance to the current therapies, for example through highly selective targeting of drug-resistant EGFR mutants.

Recent studies suggest that purposefully targeting allosteric sites might lead to mutant-selective inhibitors (Jia et al. Overcoming EGFR(T790M) and EGFR(C797S) resistance with mutant-selective allosteric inhibitors, June 2016, Nature 534, 129-132).

The field of targeted protein degradation promoted by small molecules has been intensively studied (Collins et al., Biochem J, 2017, 474(7), 1127-47). Protein degradation plays a role in various cellular functions. For example, the body uses protein degradation to adjust the concentrations of regulatory proteins through degradation into small peptides to maintain the health and productivity of the cells.

Cereblon is a protein that forms an E3 ubiquitin ligase complex, which ubiquitinates various other proteins. Cereblon is known as the primary target for the anticancer thalidomide analogs. A higher expression of cereblon has been linked to the efficiency of thalidomide analogs in cancer therapy.

Compounds have been described as useful modulators of targeted ubiquitination, for example the compounds described in. WO2013020557, WO2013063560, WO2013106643, WO/2013170147, WO2016011906, and WO/2019183523 can be used for targeted ubiquitination. Additional modulators for targeted ubiquitination include those described by Ranok Therapeutics (Hangzhou) Co. Ltd. WO2020206608 and WO2020207396 those described by Arvinas, Inc. in WO2015160845, WO2016149668, WO2016197032, WO2017011590, WO2017030814, WO2018144649, WO2018226542, and WO2019199816; those described by Dana-Farber Cancer Institute in WO2016105518, WO2017007612, WO2017024317, WO2017024318, WO2017117473, WO2017117474, WO2018148443, WO2018148440, and WO2019165229; those described by Kymera Therapeutics in WO2019/060742, WO2019/140387, and WO2020/01022; and those described by C4 Therapeutics, Inc. in WO2017197036, WO2017197046, WO2017197051, WO2017197055, WO2018237026, WO2019099868, WO2019191112, WO2019204353, WO2019236483, WO2020132561, WO2020181232, and WO2020210630.

Some specific molecules for the degradation of EGFR have also been described, for example, Dana-Farber Cancer Institute described EGFR degraders in WO2017185036. F. Hoffman-La-Roche described EGFR degraders in WO2019121562 and WO2019149922. Arvinas, Inc. has described EGFR degraders in WO2018119441. Additional EGFR Degraders have been described in the paper by Jang et al. titled “Mutant-Selective Allosteric EGFR Degraders are Effective Against a Broad Range of Drug-Resistant Mutations”, Angewandte Chemie, 59(34), 14481-489.

Despite these efforts, because of the life-threatening cancers exhibiting EGFR mutations and/or overexpression, there remains a need for new EGFR modulators to treat disorders mediated by EGFR in hosts, and in particular humans, in need thereof.

### SUMMARY OF THE INVENTION

Methods of treating an EGFR mediated cancer that has metastasized to the brain or central nervous system, for example, the peripheral nervous system, cerebral spinal fluid, spinal cord, leptomeninges, epidural space, and/or dura, are presented that comprise administering an effective amount of a compound of Formula I, II, III, or IV, or a pharmaceutically acceptable salt thereof, to a patient in need thereof. The compounds of Formula I, II, III, and IV include a Targeting Ligand that binds to EGFR, an E3 Ligase binding portion (typically via a cereblon subunit), and a Linker that covalently links the Targeting Ligand to the E3 Ligase binding portion. In certain embodiments the E3 Ligase binding portion is a moiety of A or A*, the Linker is a moiety of L¹ or L², and the remainder of the molecule is the EGFR Targeting Ligand portion.

The EGFR Targeting Ligand may be an allosteric inhibitor. Allosteric binding before degradation results in advantages to the use of the compounds of the present invention over traditional EGFR inhibitors, covalent modulators, and even non-allosteric degraders. Non-limiting examples of the advantages of using the allosteric degrading compounds described herein include increased selectivity for mutant-EGFR, increased catalytic activity, improved efficacy, the ability to overcome resistance to ATP-competitive inhibitors, and/or fewer side effects. In certain embodiments the allosteric degrading compound of the present invention effectively binds and degrades EGFR with a mutation that imparts resistance to osimertinib and/or erlotinib, for example a mutation that replaces an active site cysteine with another amino acid. Because of these advantages the compounds described herein can be used to treat cancer that has metastasized to the brain or CNS and developed resistance to osimertinib (e.g., 2^{nd} line therapy or treatment for non-small cell lung cancer). In other embodiments a compound described herein can be used to treat a cancer that has metastasized to the brain or CNS that is treatment naïve (e.g., 1^{st} line therapy or treatment for non-small cell lung cancer). In other embodiments a compound described herein can be used to treat a cancer that has metastasized to the brain or CNS and that developed resistance to multiple lines of therapy (e.g., 3rd line therapy or treatment for non-small cell lung cancer).

In other embodiments, the EGFR Targeting Ligand may be an active site inhibitor.

In certain embodiments the method provided selectively degrades EGFR in a tumor that has metastasized to the brain or CNS and may have a mutation or combination of mutations, for example a mutation selected from T790M, L858R, and C797S; the combination of two mutations selected from T⁷⁹⁰M, L858R, and C797S; or the combination of three mutations selected from T⁷⁹⁰M, L858R, and C797S. In certain embodiments the method utilizes a selective degrader of L⁸⁵⁸R-T790M, L858R-T790M-C797S, L⁸⁵⁸R, or L858R-C797S containing EGFR mutants.

In certain embodiments a method of treating an EGFR mediated cancer that has metastasized to the brain or CNS comprising administering an effective amount of Compound 1, Compound 2, Compound 3, Compound 4, Compound 5, Compound 6, Compound 7, Compound 8, Compound 9, Compound 10, Compound 11, or Compound 12, or a pharmaceutically acceptable salt thereof, to a patient in need thereof is provided. These compounds are allosteric site binding EGFR degraders (allosteric EGFR degraders).

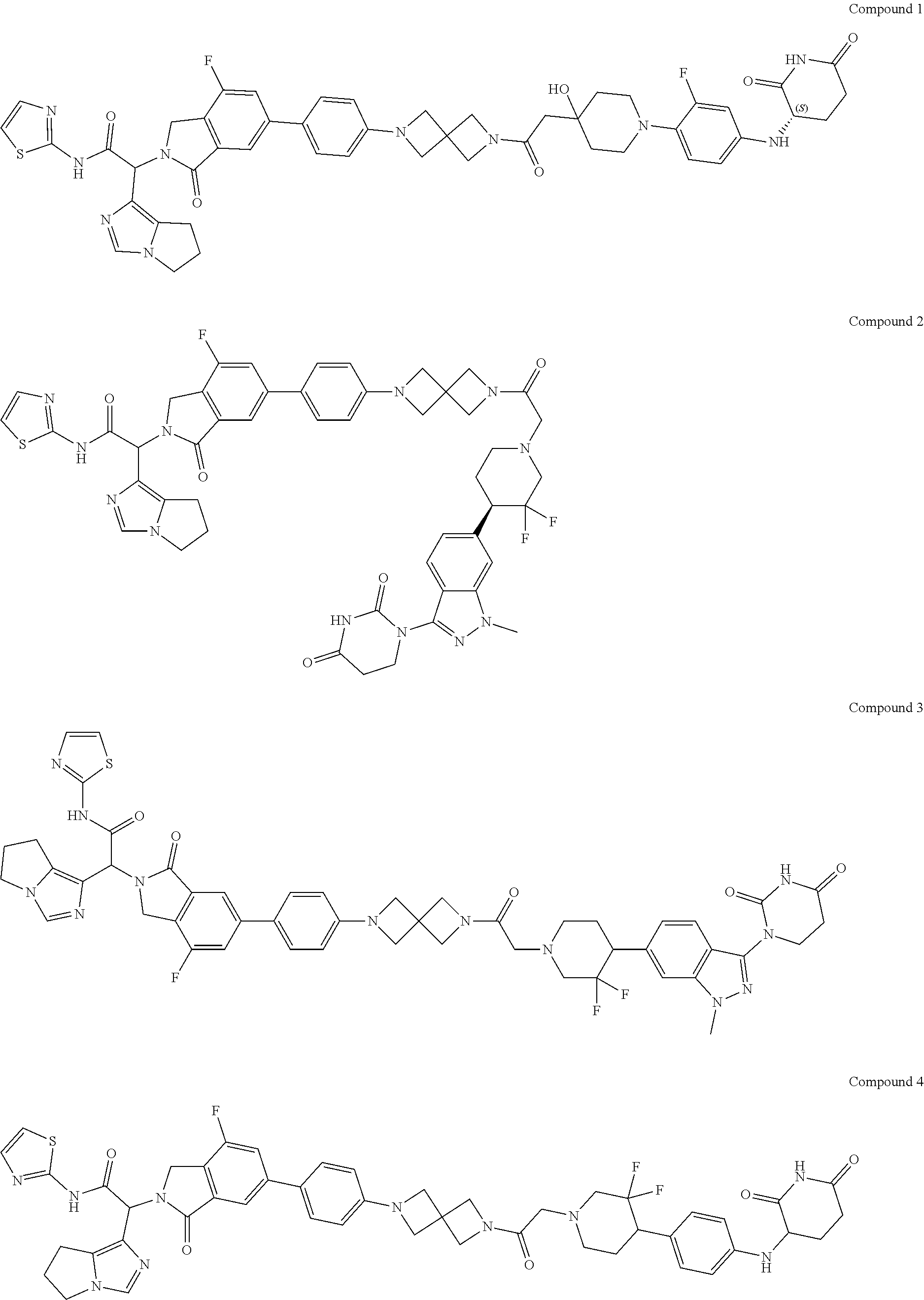

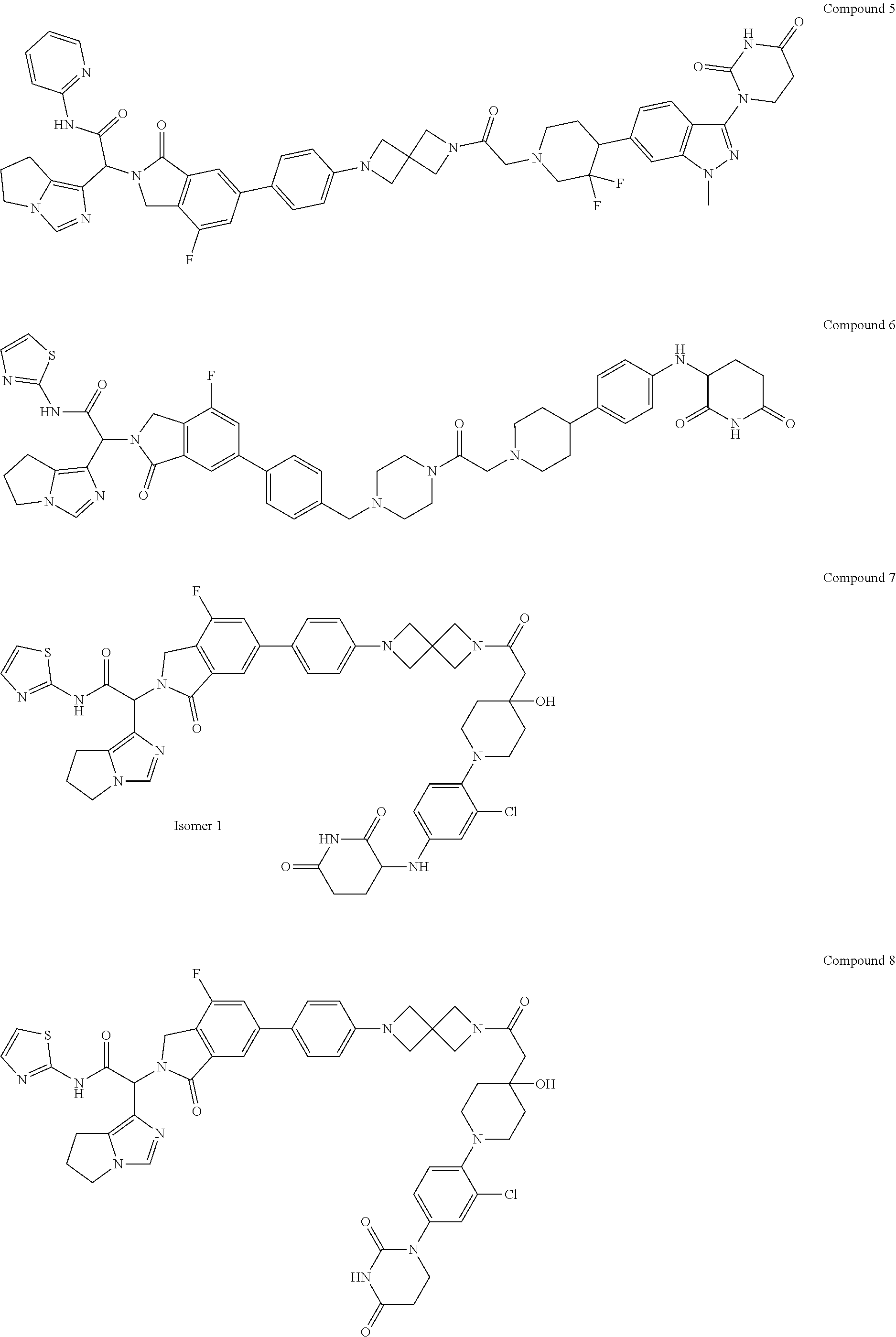

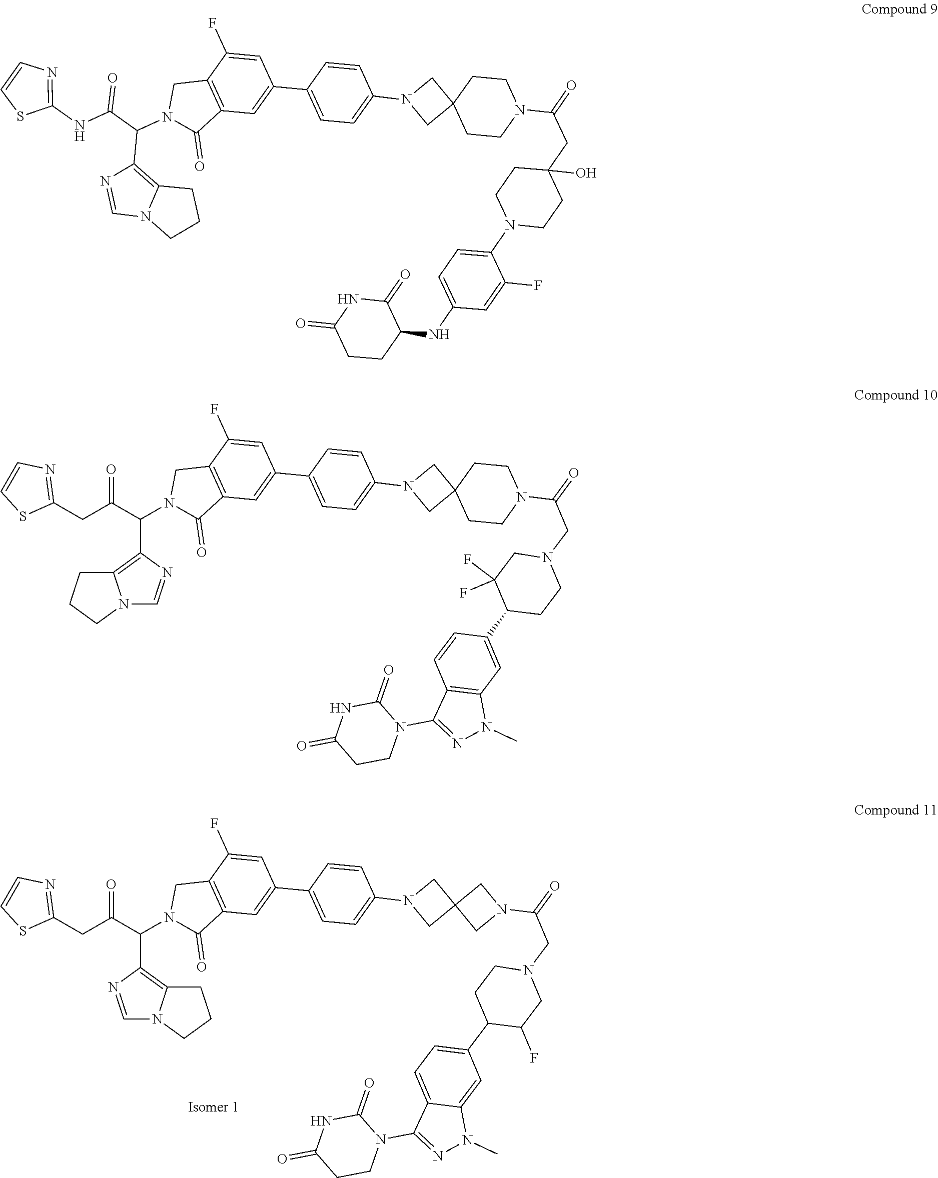 [SMILES: O=C1CC[C@H](Nc2ccc(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1 ; Cn1nc(N2CCC(=O)NC2=O)c2ccc([C@H]3CCN(CC(=O)N4CCC5(CC4)CN(c4ccc(-c6cc(F)c7c(c6)C(=O)N(C(C(=O)Cc6nccs6)c6ncn8c6CCC8)C7)cc4)C5)CC3(F)F)cc21 ; Cn1nc(N2CCC(=O)NC2=O)c2ccc(C3CCN(CC(=O)N4CC5(C4)CN(c4ccc(-c6cc(F)c7c(c6)C(=O)N(C(C(=O)Cc6nccs6)c6ncn8c6CCC8)C7)cc4)C5)CC3F)cc21]

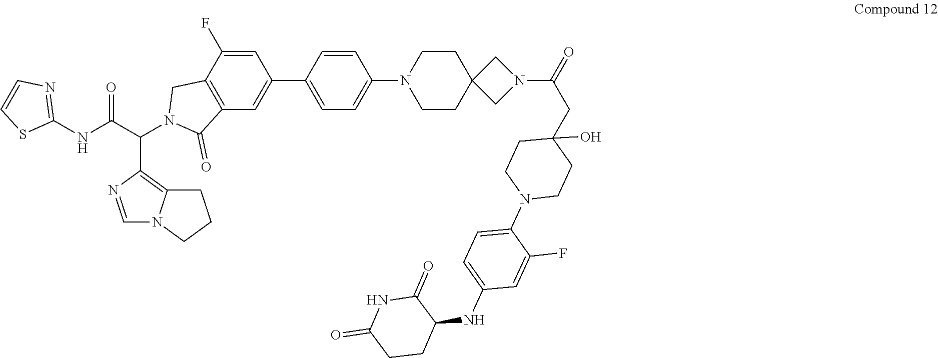 [SMILES: O=C1CC[C@H](Nc2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1]

Large concentrations of the allosteric EGFR degraders described herein cross the blood brain barrier. Compounds described herein, including for example Compound 1, are also highly selective and degrade mutant EGFR-L858R protein without appreciably degrading other non-EGFR proteins. In a kinome screen (see Example 60 and FIGS. 13A and 13B) a compound described herein had negligible binding against hundreds of proteins. Additionally, in global proteomics similarly high selectivity was observed (see Example 61 and Table 14). Further, the compounds described herein have very low activity for the degradation of SALL4 and GSPT1, two proteins that are degraded by IMID compounds such as lenalidomide and CC-885 (see FIG. 7 and FIG. 8). In NCI-H1975 (EGFR-L858R-T790M) and NCI-H3255 (EGFR-L858R) human lung cancer lines, degradation of 50% of mutant EGFR was achieved at 6 hours with nanomolar concentrations of allosteric EGFR degraders (see Table 9A). In addition, EGFR phosphorylation is potently inhibited (see Table 10A). In contrast, allosteric EGFR degraders described herein did not reach 50% degradation nor phospho-EGFR inhibition up to a concentration of 10 μM in the human wild-type EGFR cell line A431. An allosteric EGFR degrader described herein also inhibits proliferation of the engineered BaF3 cells expressing EGFR variants including L858R, L858R-C797S, L858R-T790M, or L858R-T790M-C797S EGFR mutants, with GI50 values ranging from 8 to 16 nM, compared to an GI50 of 486 nM in BaF3 cells expressing wild-type EGFR (see Table 10B).

Oral dosing of Compound 1 or Compound 2 is well tolerated in mice. Treatment with Compound 1 in an NCI-H1975 mouse xenograft model led to dose-dependent activity and up to 90% tumor regression (see FIG. 1). In addition, up to 85% of mutant EGFR was degraded in vivo after a single oral dose of Compound 1, and phospho-EGFR was decreased >95% (see FIGS. 2A and 2B). In an engineered BaF3 EGFR-L858R-T790M-C797S allograft mouse model of osimertinib resistance, oral dosing of Compound 1 led to 60% tumor regression, in contrast to osimertinib treatment where minimal efficacy was observed (see FIG. 3). In a luciferase expressing NCI-H1975 intracranial model of brain metastasis, oral dosing of Compound 1 resulted in tumor regression (see FIG. 5A). Oral dosing of Compound 1 also resulted in tumor regression in an intracranial model with intracarotid implementation (see FIG. 14).

In certain embodiments an allosteric EGFR degrader is administered as second line therapy for the treatment of EGFR-mediated cancer that has metastasized to the brain or CNS, for example, an allosteric EGFR degrader may be administered to a patient that has progressed off osimertinib. In other embodiments an allosteric EGFR degrader is administered as a first line therapy for the treatment of EGFR-mediated cancer that has metastasized to the brain or CNS. In other embodiments an allosteric EGFR degrader is administered as a third line therapy for the treatment of EGFR-mediated cancer that has metastasized to the brain or CNS.

In certain embodiments the allosteric EGFR degrader binds to the allosteric site created by the displacement of the regulatory αC-helix in an “αC-out” conformation. In this embodiment the allosteric site may be enlarged in the activation loop mutants such as Exon 21 L858R or L861Q but is occluded in wild type EGFR. This mechanism provides mutant selectivity over wild type. In certain embodiments an allosteric EGFR degrader described herein degrades mutant EGFR monomers and dimers.

In certain aspects the present invention provides a method of treating an EGFR-mediated cancer that has metastasized to the brain or CNS comprising administering an effective amount of a compound of Formula:

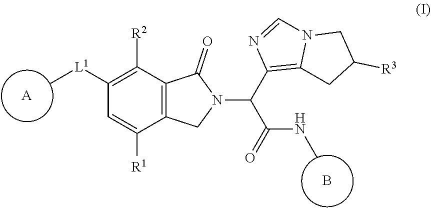

or a pharmaceutically acceptable salt, isotope, N-oxide, or stereoisomer thereof;
wherein:
   A is selected from the ring systems AF and AG;

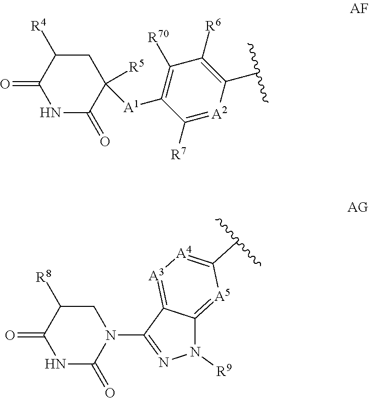

A¹ is selected from
   i) —NH—, and
   ii) —O—;
A² is selected from
   i) —N—, and
   ii) —CR⁵²—;
A³ is selected from
   i) —N—, and
   ii) —CR⁵³—;
A⁴ is selected from
   i) —N—, and
   ii) —CR⁵⁴—;
A⁵ is selected from
   i) —N—, and
   ii) —CR⁵⁵—;
R¹ is selected from
   i) H,
   ii) halogen
   iii) C₁₋₆-alkyl;
R⁵² is selected from
   i) H,
   ii) halogen,
   iii) cyano,
   iv) C₁₋₆-alkoxy,
   v) halo-C₁₋₆-alkoxy,
   vi) C₁₋₆-alkyl,
   vii) halo-C₁₋₆-alkyl,
   viii) C₃₋₈-cycloalkyl, and
   ix) halo-C₃₋₈-cycloalkyl;
R⁵³, R⁵⁴ and R⁵⁵ are independently selected from
   i) H,
   ii) halogen,
   iii) C₁₋₆-alkyl,
   iv) halo-C₁₋₆-alkyl,
   v) C₃₋₈-cycloalkyl, and
   vi) halo-C₃₋₈-cycloalkyl;
R² is selected from
   i) H,
   ii) halogen,
   iii) C₁₋₆-alkyl,
   iv) halo-C₁₋₆-alkyl,
   v) C₃₋₈-cycloalkyl, and
   vi) halo-C₃₋₈-cycloalkyl;
R³ is selected from
   i) H,
   ii) halogen,
   iii) C₁₋₆-alkyl,
   iv) halo-C₁₋₆-alkyl,
   v) C₃₋₈-cycloalkyl, and
   vi) halo-C₃₋₈-cycloalkyl;
R⁴ and R⁵ are H;
or R⁴ and R⁵ together form —(CH₂)_{q}—;
q is 1 or 2;
R⁶ is selected from
   i) H,
   ii) halogen,
   iii) cyano,
   iv) C₁₋₆-alkoxy,
   v) halo-C₁₋₆-alkoxy,
   vi) C₁₋₆-alkyl,
   vii) halo-C₁₋₆-alkyl,
   viii) C₃₋₈-cycloalkyl, and
   ix) halo-C₃₋₈-cycloalkyl;
R⁷ is selected from
   i) H,
   ii) halogen,
   iii) cyano,
   iv) C₁₋₆-alkyl,
   v) halo-C₁₋₆-alkyl,
   vi) C₃₋₈-cycloalkyl, and
   vii) halo-C₃₋₈-cycloalkyl;
R⁷⁰ is selected from
   i) H,
   ii) halogen,
   iii) cyano,
   iv) C₁₋₆-alkyl,
   v) halo-C₁₋₆-alkyl,
   vi) C₃₋₈-cycloalkyl, and
   vii) halo-C₃₋₈-cycloalkyl;
R⁸ is H;
R⁹ is selected from
   i) H, and
   ii) C₁₋₆-alkyl;
L¹ is

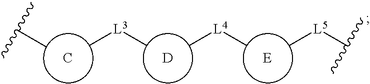

C is absent or selected from the ring systems F, G and H;

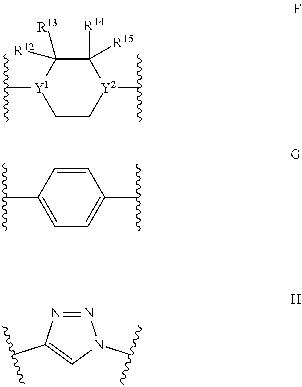

Y¹ is selected from
   i) —N—, and
   ii) —CH—;
Y² is selected from
   i) —N—, and
   ii) —CR¹⁶—;
R¹², R¹³, R¹⁴ and R¹⁵ are independently selected from
   i) —H—,
   ii) halogen, and
   iii) hydroxy-C₁₋₆-alkyl;
R¹⁶ is selected from
   i) —H—,
   ii) hydroxy, and
   iii) fluoro;
L³ is absent or selected from
   i) —(CH₂)ₘ—C(O)—,
   ii) —C(O)—(CH₂)ₚ—,
   iii) —C(O)—C(O)—,
   iv) —NR¹⁰—C(O)—,
   v) —C(O)—NR¹⁰—,
   vi) —C(O)O—,
   vii) —CH₂—CF₂—CH₂—,
   viii) —CH₂—,

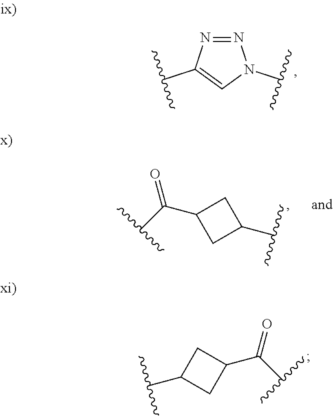 [SMILES: *c1cn(*)nn1 ; *C(=O)C1CC(*)C1 ; *C(=O)C1CC(*)C1]

m is 0, 1 or 2;
p is 0, 1, 2 or 3;
R¹⁰ is selected from
   i) H, and
   ii) C₁₋₆-alkyl;
D is selected from the ring systems I, J, K, L, M, N, O, P, Q, R, S, T, U, V, W and X, all ring systems being optionally substituted by one to three substituents selected from R⁸⁰, R⁸¹ and R⁸²;

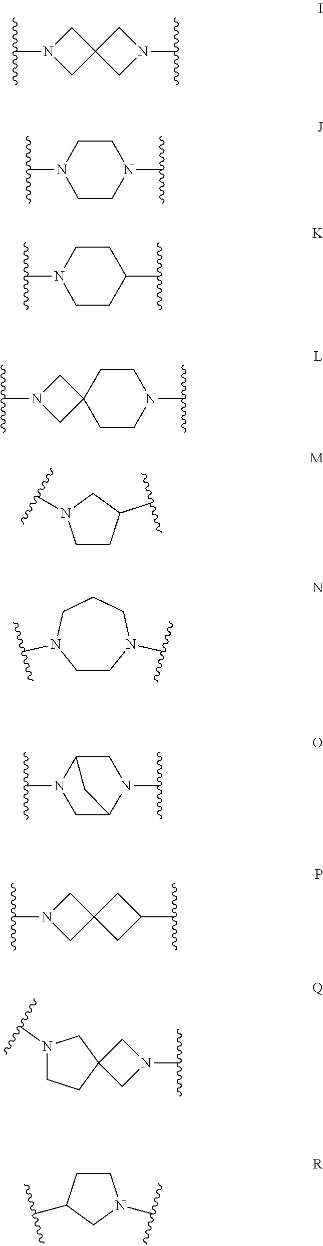

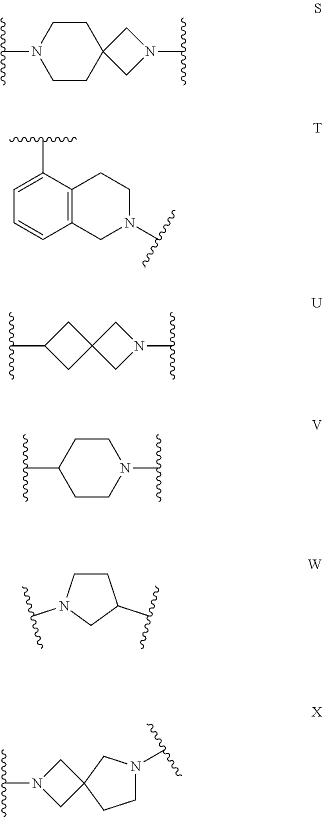 [SMILES: *N1CCC2(CC1)CN(*)C2 ; *c1cccc2c1CCN(*)C2 ; *C1CC2(C1)CN(*)C2 ; *C1CCN(*)CC1 ; *C1CCN(*)C1 ; *N1CCC2(C1)CN(*)C2]

R⁸⁰, R⁸¹ and R⁸² are independently selected from
   i) halogen,
   ii) cyano,
   iii) hydroxy,
   iv) hydroxy-C₁₋₆-alkyl,
   v) C₁₋₆-alkoxy,
   vi) halo-C₁₋₆-alkoxy,
   vii) C₁₋₆-alkyl,
   viii) halo-C₁₋₆-alkyl,
   ix) C₃₋₈-cycloalkyl, and
   x) halo-C₃₋₈-cycloalkyl;
L⁴ is absent or selected from
   i) —NR¹¹—C(O)—,
   ii) —CH₂—, and
   iii) —O—;
E is selected from the ring systems Y, Z, AA, AB and AC;

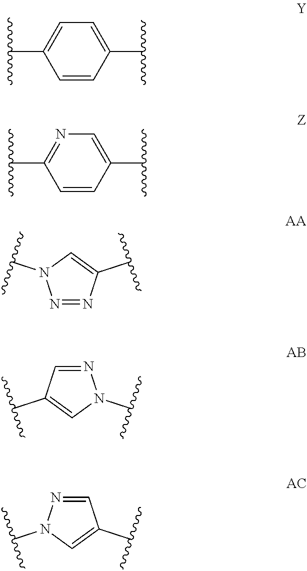 [SMILES: *c1ccc(*)cc1 ; *c1ccc(*)nc1 ; *c1cn(*)nn1 ; *c1cnn(*)c1 ; *c1cnn(*)c1]

L5 is absent or

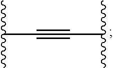 [SMILES: *C#C*]

B is selected from the ring system AD and AE;

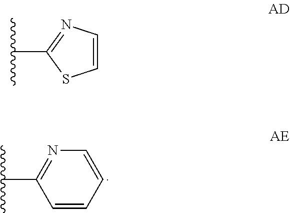 [SMILES: *c1nccs1 ; *c1ccccn1]

In certain aspects the present invention provides a method of treating an EGFR-mediated cancer that has metastasized to the brain or CNS comprising administering an effective amount of a compound of Formula:

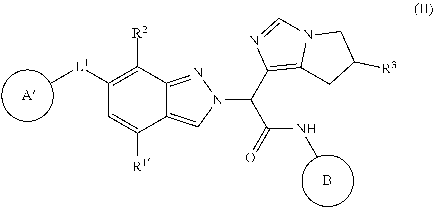

or a pharmaceutically acceptable salt, isotope, N-oxide, or stereoisomer thereof;
   wherein
   A′ is selected from the ring systems AF, AG and AH;

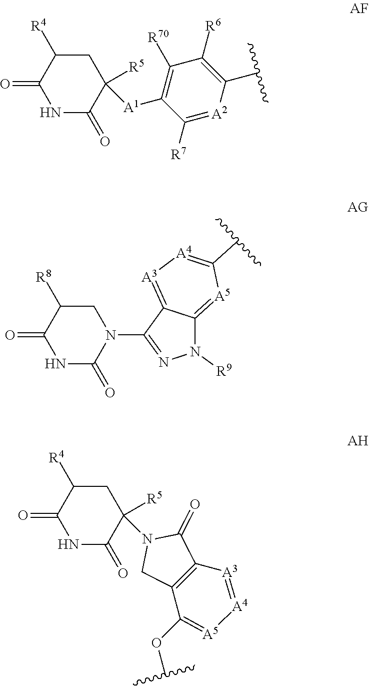

R^{1′} is selected from
   i) H,
   ii) halogen,
   iii) C₁₋₆-alkyl
   iv) cyano,
   v) C₁₋₆-alkoxy,
   vi) halo-C₁₋₆-alkoxy,
   vii) C₁₋₆-alkyl,
   viii) halo-C₁₋₆-alkyl,
   ix) C₃₋₈-cycloalkyl, and
   x) halo-C₃₋₈-cycloalkyl;
and the remaining variables are as defined herein.

In other aspects the present invention provides a method of treating an EGFR-mediated cancer that has metastasized to the brain or CNS comprising administering an effective amount of a compound of Formula:

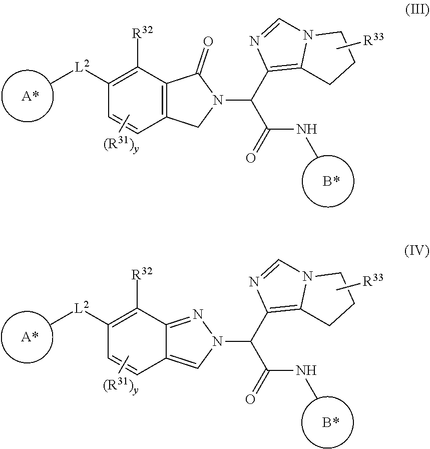

or a pharmaceutically acceptable salt, isotope, N-oxide, or stereoisomer thereof;
wherein:
   A* is selected from:

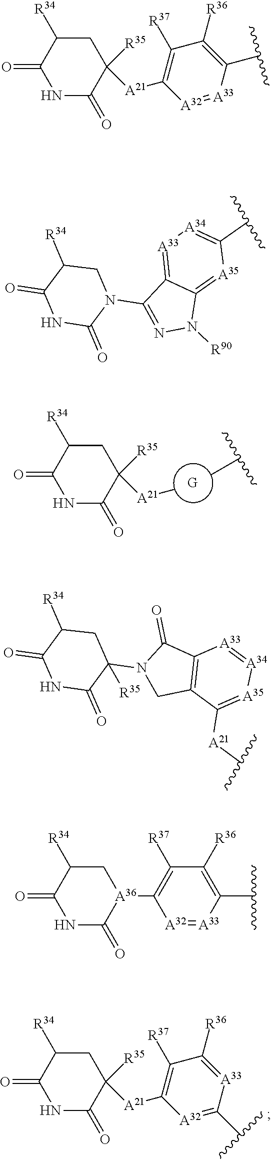

B* is heteroaryl or aryl which is optionally substituted with 1, 2, or 3 R³¹ substituents; in certain embodiments B* is selected from

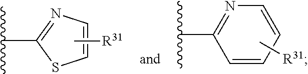

y is 0, 1, 2, or 3;
R³¹ is independently selected at each occurrence from H, halogen (F, Cl, Br, or I), C₁₋₆-alkyl, cyano, C₁₋₆-alkoxy, halo-C₁₋₆-alkoxy, halo-C₁₋₆-alkyl, C₃₋₈-cycloalkyl, and halo-C₃₋₈-cycloalkyl and can be located on either ring where present on a bicycle, for example

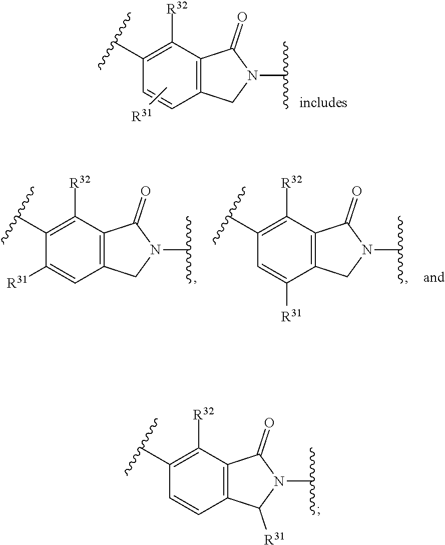

R³² is hydrogen, halogen (F, Cl, Br, or I), C₁₋₆-alkyl, halo-C₁₋₆-alkyl, C₃₋₈-cycloalkyl, or halo-C₃₋₈-cycloalkyl;
R³³ is hydrogen, halogen (F, Cl, Br, or I), C₁₋₆-alkyl, halo-C₁₋₆-alkyl, C₃₋₈-cycloalkyl, or halo-C₃₋₈-cycloalkyl and can be located on the dihydropyrrole or imidazole ring;
R³⁴ is independently selected at each occurrence from H, F, C₁₋₆-alkyl, halo-C₁₋₆-alkyl, C₃₋₈-cycloalkyl, and halo-C₃₋₈-cycloalkyl;
R³⁵ is selected at each occurrence from H, halogen (F, Cl, Br, or I), C₁₋₆-alkyl, halo-C₁₋₆-alkyl, and C₃₋₈-cycloalkyl;
or R³⁴ and R³⁵ combine to form —(CH₂)_{q}—;
R³⁶ and R³⁷ are independently selected from H, halogen (F, Cl, Br, or I), cyano, C₁₋₆-alkoxy, halo-C₁₋₆-alkoxy(for example F, Cl, or Br), C₁₋₆-alkyl, halo-C₁₋₆-alkyl (for example F, Cl, or Br), C₃₋₈-cycloalkyl, and halo-C₃₋₈-cycloalkyl;
or R³⁶ and R³⁷ together are combined to form a 5- or 6-membered cycle optionally substituted with 1, 2, or 3 R³¹ substituents;
R⁴² is independently selected at each occurrence from H, halogen (F, Cl, Br, or I), cyano, C₁₋₆-alkoxy, halo-C₁₋₆-alkoxy, C₁₋₆-alkyl, halo-C₁₋₆-alkyl, C₃₋₈-cycloalkyl, and halo-C₃₋₈-cycloalkyl;
R⁹⁰ is H, C₁₋₆-alkyl, or C₃₋₆-cycloalkyl;
Ring G is a heteroaryl optionally substituted with 1 or 2 R⁴² substituents, for example a 5- or 6-membered heteroaryl ring with 1, 2, or 3 N heteroatoms;
A²¹ is —NH—, —O—, —CH₂—, or —NR¹⁰⁰—;
R¹⁰⁰ is alkyl, cycloalkyl, aryl, or heteroaryl; or as allowed by valence R¹⁰⁰ may combine with R³⁷ to form a 5-8 membered heterocycle or 5 membered heteroaryl;
A³², A³³, A³⁴, and A³⁵ are independently selected from —N— and —CR⁴²—;
A³⁶ is —N— or —CR³⁵—;
L² is a bivalent linking group (a linker) that connects A* and either the isoindolinone or indazole, for example but not limited to a bivalent linking group of Formula LI; and wherein the remaining variables are as defined herein.

In certain embodiments L² is of formula:

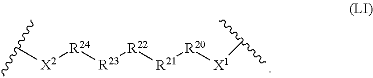

wherein,
   X¹ and X² are independently at each occurrence selected from bond, heterocycle, aryl, heteroaryl, bicycle, alkyl, aliphatic, heteroaliphatic, —NR²⁷—, —CR⁴⁰R⁴¹—, —O—, —C(O)—, —C(NR²⁷)—, —C(S)—, —S(O)—, —S(O)₂— and —S—; each of which heterocycle, aryl, heteroaryl, and bicycle is optionally substituted with 1, 2, 3, or 4 substituents independently selected from R⁴⁰;
   R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, oxyalkylene, —C(R⁴⁰R⁴⁰)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, and carbocycle; each of which is optionally substituted with 1, 2, 3, or 4 substituents independently selected from R⁴⁰;
   R²⁶ is independently at each occurrence selected from the group consisting of hydrogen, alkyl, arylalkyl, heteroarylalkyl, alkene, alkyne, aryl, heteroaryl, heterocycle, aliphatic and heteroaliphatic;
   R²⁷ is independently at each occurrence selected from the group consisting of hydrogen, alkyl, aliphatic, heteroaliphatic, heterocycle, aryl, heteroaryl, —C(O)(aliphatic, aryl, heteroaliphatic or heteroaryl), —C(O)O(aliphatic, aryl, heteroaliphatic, or heteroaryl), alkene, and alkyne;
   R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(aliphatic, including alkyl), —N(aliphatic, including alkyl)₂, —NHSO₂(aliphatic, including alkyl), —N(aliphatic, including alkyl)SO₂alkyl, —NHSO₂(aryl, heteroaryl or heterocycle), —N(alkyl)SO₂(aryl, heteroaryl or heterocycle), —NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heterocycle, oxo, and cycloalkyl; additionally, where allowed by valence two R⁴⁰ groups bound to the same carbon may be joined together to form a 3-8 membered spirocycle; and
   R⁴¹ is aliphatic, aryl, heteroaryl, or hydrogen.

Every combination of variables, substituents, embodiments and the methods that result from these combinations, is deemed specifically and individually disclosed, as such depiction is for convenience of space only.

In certain embodiments a compound of Formula I, II, III, or IV is an allosteric degrader of EGFR. For example, the compound may bind an allosteric site on EGFR, for example mutated EGFR) and then direct degradation of the EGFR protein.

In certain embodiments a compound of Formula I, II, III, or IV crosses the blood brain barrier. By crossing the blood brain barrier the compound of Formula I, II, III, or IV can be used to treat an EGFR-mediated cancer that has metastasized to the brain or CNS. Non-limiting examples of EGFR-mediated cancers include non-small cell lung cancer; breast cancer, including HER-2 positive breast cancer, ER+ (estrogen positive) breast cancer, PR+ (progesterone positive) breast cancer, or triple negative breast cancer; head and neck cancer; glioblastoma; pancreatic cancer; thyroid cancer; astrocytoma; esophageal cancer; cervical cancer; synovial sarcoma; ovarian cancer; liver cancer; bladder cancer; and kidney cancer.

In certain embodiments a compound described herein is used to treat lung cancer that has metastasized to the brain or CNS. In certain embodiments, the lung cancer is non-small cell lung cancer that has metastasized to the brain or CNS. In certain embodiments, the lung cancer is small cell lung cancer that has metastasized to the brain or CNS. In certain embodiments, the lung cancer is adenocarcinoma that has metastasized to the brain or CNS. In certain embodiments, the lung cancer is squamous cell lung cancer that has metastasized to the brain or CNS. In certain embodiments, the lung cancer is large-cell undifferentiated carcinoma that has metastasized to the brain or CNS. In certain embodiments, the lung cancer is neuroendocrine carcinoma that has metastasized to the brain or CNS. Additional examples of lung cancers include sarcomatoid carcinoma, adenosquamous carcinoma, oat-cell cancer, combined small cell carcinoma, lung carcinoid tumor, central carcinoid, peripheral carcinoid, salivary gland-type lung carcinoma, mesothelioma, and mediastinal tumors.

In certain embodiments a compound described herein is used to treat breast cancer that has metastasized to the brain or CNS. In certain embodiments, the breast cancer is HER-2 positive breast cancer. In certain embodiments, the breast cancer is ER+ breast cancer. In certain embodiments, the breast cancer is PR+ breast cancer. In certain embodiments, the breast cancer is triple negative breast cancer.

In certain embodiments a compound described herein is used to treat colorectal or rectal cancer that has metastasized to the brain or CNS.

In certain embodiments a compound described herein is used to treat head and neck cancer or esophageal cancer that has metastasized to the brain or CNS.

In certain embodiments a compound described herein is used to treat pancreatic cancer that has metastasized to the brain or CNS.

In certain embodiments a compound described herein is used to treat thyroid cancer that has metastasized to the brain or CNS.

In certain embodiments a compound described herein is used to treat ovarian cancer, uterine cancer, or cervical cancer that has metastasized to the brain or CNS.

In certain embodiments a compound described herein is used to treat kidney cancer, liver cancer, or bladder cancer that has metastasized to the brain or CNS.

In certain embodiments a compound described herein is used to treat melanoma that has metastasized to the brain or CNS.

In certain embodiments a compound described herein is used to treat kidney cancer, liver cancer, or bladder cancer that has metastasized to the brain or CNS. In other embodiments, the compound is used to treat adenocarcinoma, colorectal carcinoma, breast cancer, triple negative breast cancer, renal cell carcinoma, a primary brain tumor, astrocytoma, esophageal cancer or synovial sarcoma

In certain embodiments, a compound described herein crosses the blood brain barrier in sufficient concentrations to treat an EGFR-mediated disorder such as cancer in the brain or CNS and has one or more, and even may provide multiple additional advantages over traditional treatment with an EGFR inhibitor. For example, the EGFR degrading compound described herein may a) overcome resistance in certain cases; b) prolong the kinetics of drug effect by destroying the protein, thus requiring resynthesis of the protein even after the compound has been metabolized; c) target all functions of a protein at once rather than a specific catalytic activity or binding event; and/or d) have increased potency compared to inhibitors due to the possibility of the small molecule acting catalytically.

In one aspect, a compound described herein is used to treat an EGFR mediated cancer that has metastasized to the brain or CNS, wherein the EGFR has mutated from the wild-type. There are a number of possibilities for EGFR mutations. In certain non-limiting embodiments, the mutation is found in exon 18, exon 19, exon 20, or exon 21, or any combination thereof. In certain nonlimiting embodiments, the mutation is at position L858, E709, G719, C797, L861, T790, or L718 or any combination thereof. In certain embodiments the mutation is a L858R, T790M, L718Q, L792H, and/or a C797S mutation or any combination thereof.

In certain aspects, the cancer has developed one or more EGFR mutations following treatment with at least one EGFR inhibitor that can be a non-covalent inhibitor (including but not limited to gefitinib, erlotinib, lapatinib or vandetanib) or a covalent inhibitor (such as afatinib, osimertinib or dacomitinib). In another aspect, the cancer has developed one or more EGFR mutations following treatment with an antibody such as cetuximab, panitumab or necitumab. In yet another aspect, the cancer has one or more EGFR mutations or non-EGFR mutations that renders the cancer intrinsically resistant to EGFR inhibitor treatment, for example, a somatic exon 20 insertion, asomatic PIK3CA mutation, loss of PTEN expression, MET amplification, or a KRAS mutation.

In certain embodiments, a compound described herein is used to treat a cancer that has metastasized to the brain or CNS that is resistant to, or has acquired a resistance to, a first generation EGFR inhibitor such as erlotinib, gefitinib, and/or lapatinib. In certain embodiments, a compound described herein is used to treat a cancer that is resistant to, or has acquired a resistance to a second generation EGFR inhibitor such as afatinib and/or dacomitinib. In certain embodiments, a compound described herein is used to treat a cancer that is resistant to, or acquired a resistance to a third generation EGFR inhibitor such as osimertinib.

In some embodiments, the mutated EGFR protein in the diseased tissue has an L858 mutation, for example L858R.

In certain embodiments a compound described herein is used to treat a mutant EGFR-mediated cancer that has metastasized to the brain or CNS, wherein EGFR has a mutation of at least one of the below listed amino acid sites, or a combination thereof. The mutation may, for example, be selected from one of the listed exemplary mutations, or may be a different mutation.

| Amino Acid | Exemplary Mutations |
|---|---|
| C797 | C797S |
| E709 | E709A, E709G, E709K, E709V |
| G719 | G719A, G719S, G719C, G719D |
| G724 | G724S |
| G119 | G119A |
| G796 | G796S, G796C |
| L718 | L718V, L718Q |
| L792 | L792H; L792V |
| L858 | L858R |
| L861 | L861Q |
| S768 | S768I |
| T790 | T790M |

In certain embodiments the mutant EGFR-mediated cancer that has metastasized to the brain or CNS has two mutations selected from the table above. In other embodiments the mutant EGFR-mediated cancer that has metastasized to the brain or CNS has three mutations selected from the table above. In other embodiments the mutant EGFR-mediated cancer that has metastasized to the brain or CNS has four or more mutations, which may optionally be selected from the table above.

In certain embodiments the mutant EGFR-mediated cancer that has metastasized to the brain or CNS has an L858R mutation and one additional mutation which may optionally be selected from the table above. In some of these embodiments the mutant EGFR-mediated cancer that has metastasized to the brain or CNS has an L858R mutation and two additional mutations that may optionally be selected from the table above. In other embodiments the mutant EGFR-mediated cancer that has metastasized to the brain or CNS has a L858R mutation and three additional mutations that may optionally be selected from the table above.

In certain embodiments the mutant EGFR-mediated cancer that has metastasized to the brain or CNS has a T790M mutation and one additional mutation optionally selected from the table above. In other embodiments the mutant EGFR-mediated cancer that has metastasized to the brain or CNS has a T790M mutation and two additional mutations optionally selected from the table above. In other embodiments the mutant EGFR-mediated cancer that has metastasized to the brain or CNS has a T790M mutation and three additional mutations optionally selected from the table above.

In certain embodiments the mutant EGFR-mediated cancer that has metastasized to the brain or CNS has a L718Q mutation and one additional mutation optionally selected from the table above. In other embodiments the mutant EGFR-mediated cancer that has metastasized to the brain or CNS has a L718Q mutation and two additional mutations optionally selected from the table above. In other embodiments the mutant EGFR-mediated cancer that has metastasized to the brain or CNS has a L718Q mutation and three additional mutations optionally selected from the table above.

In certain embodiments the mutant EGFR-mediated cancer that has metastasized to the brain or CNS has a mutation of S768I, L718V, L792H, L792V, G796S, G796C, G724S, and/or G719A.

In certain embodiments, a compound described herein is used to treat an EGFR-mediated cancer that has metastasized to the brain or CNS that has a frameshift mutation, for example a short in-frame deletion. In certain embodiments, a compound described herein is used to treat an EGFR-mediated cancer that has metastasized to the brain or CNS wherein the EGFR has an exon 19 deletion. In certain embodiments, the exon 19 deletion is a deletion which includes the amino acids LREA (L747-A750). In certain embodiments, the exon 19 deletion is a deletion which includes the amino acids ELREA (E746-A750).

In certain embodiments a compound described herein is used to treat an EGFR-mediated cancer that has metastasized to the brain or CNS wherein the EGFR has an L858R mutation in exon 21.

In certain embodiments a compound described herein is more active against a disorder driven by a mutated EGFR than wild-type EGFR.

In certain embodiments, a compound described herein is used to treat EGFR-mediated cancer that has metastasized to the brain or CNS wherein the EGFR has one or more exon 18 deletions.

In certain embodiments a compound described herein is used to treat an EGFR-mediated cancer that has metastasized to the brain or CNS with a E709 mutation, for example E709A, E709G, E709K, or E709V.

In certain embodiments a compound described herein is used to treat an EGFR-mediated cancer that has metastasized to the brain or CNS with a L718 mutation, for example L718Q.

In certain embodiments a compound described herein is used to treat an EGFR-mediated cancer that has metastasized to the brain or CNS with a G719 mutation, for example G719S, G719A, G719C, or G719D.

In certain embodiments, a compound described herein is used to treat an EGFR-mediated cancer that has metastasized to the brain or CNS wherein the EGFR has one or more exon 19 insertions and/or one or more exon 20 insertions.

In certain embodiments, a compound described herein is used to treat a 57681 mutant EGFR-mediated cancer that has metastasized to the brain or CNS. In certain embodiments a compound described herein is used to treat a EGFR L861Q mutant EGFR-mediated cancer that has metastasized to the brain or CNS. In certain embodiments, a compound described herein is used to treat C797S mutant EGFR-mediated cancer that has metastasized to the brain or CNS.

In certain embodiments a compound described herein is used to treat a L858R-T790M mutant EGFR-mediated cancer that has metastasized to the brain or CNS.

In certain embodiments a compound described herein is used to treat a L858R-L718Q mutant EGFR-mediated cancer that has metastasized to the brain or CNS.

In certain embodiments a compound described herein is used to treat a L858R-L792H, mutant EGFR-mediated cancer that has metastasized to the brain or CNS.

In certain embodiments a compound described herein is used to treat a L858R-C797S, mutant EGFR-mediated cancer that has metastasized to the brain or CNS.

In certain embodiments a compound described herein is used to treat a L858R-T790M-C797S mutant EGFR-mediated cancer that has metastasized to the brain or CNS.

Other features and advantages of the present application will be apparent from the following detailed description.

The present invention thus includes at least the following features:
(a) A method for treating an EGFR mediated cancer which has metastasized to the brain or CNS comprising administering an effective amount of a compound of Formula I, II, III, or IV, or pharmaceutically acceptable salt thereof, as described herein, to a patient in need thereof;
(b) The method of (a) wherein the patient is also administered an ATP site binding EGFR inhibitor, for example osimertinib;
(c) Use of a compound of Formula I, II, III, or IV, or a pharmaceutically acceptable salt thereof, in an effective amount in the treatment of a patient in need thereof, typically a human, with an EGFR-mediated cancer, wherein the cancer has metastasized to the brain or CNS;
(d) The use of (c) wherein the patient is also administered an ATP site binding EGFR inhibitor, for example osimertinib;
(e) A compound of Formula I, II, III, or IV, or a pharmaceutically acceptable salt thereof, for use in the manufacture of a medicament for the treatment of a patient in need thereof, typically a human, with an EGFR-mediated cancer, wherein the cancer has metastasized to the brain or CNS;
(f) The compound of (e) wherein the patient is also administered an ATP site binding EGFR inhibitor, for example osimertinib;
(g) A method for treating a mutant EGFR mediated cancer which has metastasized to the brain comprising administering an effective amount of a compound of Formula I, II, III, or IV, or pharmaceutically acceptable salt thereof, as described herein, to a patient in need thereof;
(h) The method of (g) wherein the patient is also administered an ATP site binding EGFR inhibitor, for example osimertinib;
(i) Use of a compound of Formula I, II, III, or IV, or a pharmaceutically acceptable salt thereof, in an effective amount in the treatment of a patient in need thereof, typically a human, with a mutant EGFR-mediated cancer, wherein the cancer has metastasized to the brain or CNS;
(j) The use of (i) wherein the patient is also administered an ATP site binding EGFR inhibitor, for example osimertinib;
(k) A compound of Formula I, II, III, or IV, or a pharmaceutically acceptable salt thereof, for use in the manufacture of a medicament for the treatment of a patient in need thereof, typically a human, with a mutant EGFR-mediated cancer, wherein the cancer has metastasized to the brain or CNS;

(l) The compound of (k) wherein the patient is also administered an ATP site binding EGFR inhibitor, for example osimertinib.

### BRIEF DESCRIPTION OF THE FIGURES

FIG. 1A is a line graph demonstrating the in vivo efficacy of Compound 1 or osimertinib in the treatment of female BALB/c nude mice bearing NCI-H1975 L858R-T790M NSCLC xenograft tumors. Mice were treated with the vehicle control, a dose response (20, 50 and 100 mg/kg/day) of Compound 1, or 25 mg/kg/day of osimertinib for 14 days. Compound 1 was administered orally (PO) on a twice a day basis (BID) and osimertinib was administered orally (PO) on a once per day basis (QD). The x-axis is the time measured in days and the y-axis is NCI-H1975 tumor volume measured in mm³. The experimental procedure is provided in Example 55.

FIG. 1B is a line graph demonstrating the effect on body weight of Compound 1 or osimertinib in the treatment of female BALB/c nude mice bearing NCI-H1975 NSCLC xenograft tumors. Mice were treated with the vehicle control, a dose response (20, 50 and 100 mg/kg/day) of Compound 1, or 25 mg/kg/day of osimertinib for 14 days. Compound 1 was administered orally (PO) on a twice a day basis (BID) and osimertinib was administered orally (PO) on a once per day basis (QD). After 14 days of dosing, tumors were monitored for regrowth. The x-axis is the time measured in days and the y-axis is body weight change measured as %. The experimental procedure is provided in Example 55.

FIG. 2A and FIG. 2B are graphs of the relative protein expression of (A) mutant EGFR-L858R-T790M and (B) phospho-EGFR in NCI-H1975 tumors. BALB/c nude mice were injected with NCI-H1975 tumor cells and Compound 1 was administered as a single oral (PO) dose at 10, 25, or 50 mg/kg and osimertinib was administered orally (PO) at 25 mg/kg. The x-axis is time measured in hours and represents time post-single dose administration and the y-axis is the percent of protein relative to the vehicle control normalized to alpha-tubulin. The experimental procedure is provided in Example 56.

FIG. 3A is a line graph demonstrating the in vivo efficacy of Compound 1 or osimertinib in the treatment of female BALB/c nude mice bearing engineered triple mutant EGFR (L858R-T790M-C797S) BaF3 tumors. Mice were treated with the vehicle control, a dose response (20, 50, and 100 mg/kg/day) of Compound 1, or 25 mg/kg/day of osimertinib for 14 days. All compounds were administered orally (PO) on a twice a day basis (BID) for Compound 1 and once a day basis (QD) for osimertinib. The x-axis is the time measured in days and the y-axis is BaF3 tumor volume measured in mm³. The experimental procedure is provided in Example 57.

FIG. 3B is a line graph demonstrating the effect on body weight of Compound 1 or osimertinib in the treatment of female BALB/c nude mice bearing engineered triple mutant EGFR (L858R-T790M-C797S) BaF3 tumors. Mice were treated with the vehicle control, a dose response (20, 50, and 100 mg/kg/day) of Compound 1, or 25 mg/kg/day of osimertinib for 14 days. All compounds were administered orally (PO) on a twice a day basis (BID) for Compound 1 and once a day basis (QD) for osimertinib. The x-axis is the time measured in days and the y-axis is body weight change measured in %. The experimental procedure is provided in Example 57.

FIG. 4A is a line graph showing the in vivo efficacy of Compound 2 and osimertinib in the treatment of female BALB/c nude mice bearing engineered triple mutant EGFR (L858R-T790M-C797S) BaF3 tumors. Mice were treated with the vehicle control, a dose response (20, 50, and 100 mg/kg/day) of compound 2, or 25 mg/kg/day of osimertinib for 14 days. All compounds were administered orally (PO) on a twice a day basis (BID) for Compound 2 and once a day basis (QD) for osimertinib. The x-axis is the time measured in days and the y-axis is BaF3 tumor volume measured in mm³. The experimental procedure is provided in Example 57.

FIG. 4B is a line graph showing the change in body weight caused by Compound 2 and osimertinib in the treatment of female BALB/c nude mice bearing engineered triple mutant EGFR (L858R-T790M-C797S) BaF3 tumors. Mice were treated with the vehicle control, a dose response (20, 50, and 100 mg/kg/day) of compound 2, or 25 mg/kg/day of osimertinib for 14 days. All compounds were administered orally (PO) on a twice a day basis (BID) for Compound 2 and once a day basis (QD) for osimertinib. The x-axis is the time measured in days and the y-axis is body weight change in percent. The experimental procedure is provided in Example 57.

FIG. 5A is the mean in vivo efficacy of Compound 1 in the treatment of female BALB/c nude mice bearing intracranial NCI-H1975-luciferase expressing NSCLC tumors established by injecting tumor cells intracranially into the forebrain. Mice were treated with the vehicle control, Compound 1 at 100 mg/kg for 14 days. Compound 1 was administered orally (PO) on a twice a day basis (BID). The x-axis is the time measured in days and the y-axis is NCI-H1975-luc BLI (total bioluminescence signal) measured in photons/s×10⁶. The experimental procedure is provided in Example 58.

FIG. 5B is a line graph demonstrating the mean effect on body weight for Compound 1, in the treatment of female BALB/c nude mice bearing intracranial NCI-H1975-luciferase expressing NSCLC tumors established by injecting tumor cells intracranially into the forebrain. Mice were treated with the vehicle control or Compound 1 at 100 mg/kg. Compound 1 was administered orally (PO) on a twice a day basis (BID). The x-axis is the time measured in days and the y-axis is % change of bodyweight. The experimental procedure is provided in Example 58.

FIG. 6 is a line graph of the mean plasma and tumor concentration time profile of Compound 1 following a single oral dose at 50 mg/kg. Female BALB/c nude mice were injected intracranially with NCI-H1975 (EGFR-L858R-T790M) luciferase-expressing cells and administered a single oral dose of Compound 1. Plasma and tumors were harvested at the indicated time points and injected into the LC/MS/MS system for quantitative analysis.

FIG. 7 is a dose-response curve describing the effect of Compound 1 on Sal-like protein 4 (SALL4) degradation compared to lenalidomide. The x-axis is the concentration of Compound 1 or lenalidomide in nM and the y-axis is the % SALL4 remaining after 6 hours. Compound 1 had no effect on SALL4 protein level up to 10 μM. The experimental procedure is provided in Example 62.

FIG. 8 is a dose-response curve describing the effect of Compound 1 on G1 to S Phase Transition 1 (GSPT1) degradation compared to CC-885. The x-axis is the concentration of Compound 1 or CC-885 IMiD in nM and the y-axis is the % GSPT1 remaining after 6 hours. Compound 1 had no significant effect on GSPT1 up to 10 WV. The experimental procedure is provided in Example 63.

FIG. 9 is a density map of tert-Butyl 2-[1-[4-[[(3S)-2,6-dioxo-3-piperidyl]amino]-2-fluoro-phenyl]-4-hydroxy-4-piperidyl]acetate established by X-ray diffraction. This crystal structure establishes the chirality of Compound 1 as discussed in the synthesis of Compound 1 below.

FIG. 10 is a density map of tert-Butyl 2-[1-[4-[[(3S)-2,6-dioxo-3-piperidyl]amino]-2-fluoro-phenyl]-4-hydroxy-4-piperidyl]acetate established by X-ray diffraction. This crystal structure establishes the chirality of Compound 1 as discussed in the synthesis of Compound 1 below.

FIG. 11 is a density map of tert-Butyl (4R)-4-[3-(2,4-dioxohexahydropyrimidin-1-yl)-1-methyl-indazol-6-yl]-3,3-difluoro-piperidine-1-carboxylate established by X-ray diffraction. This crystal structure establishes the chirality of Compound 2 as discussed in the synthesis of Compound 2 below.

FIG. 12 is the crystal structure of tert-Butyl (4R)-4-[3-(2,4-dioxohexahydropyrimidin-1-yl)-1-methyl-indazol-6-yl]-3,3-difluoro-piperidine-1-carboxylate established by X-ray diffraction. This crystal structure establishes the chirality of Compound 2 as discussed in the synthesis of Compound 2 below.

FIG. 13A and FIG. 13B are human kinome phylogenetic tree binding plots showing the binding selectivity of 100 nM of Compound 1 against various proteins from a panel of 486 wild-type and mutant human protein kinases. Each kinase is marked as a circle. Dark colored and light-colored circles indicate kinases with <50% and >50% percent binding remaining, respectively. The size of dark circles indicates higher-affinity binding. The smaller dark circle is EGFR-L858R and the larger dark circle is EGFR-L861Q. The experimental procedure is provided in Example 60.

FIG. 14 is a graph illustrating in vivo efficacy of Compound 1 in female BALB/c nude mice bearing intracranial NCI-H1975-luciferase expressing tumors established by injecting tumor cells in the carotid artery. Mice were treated with the vehicle control, Compound 1 at 100 mg/kg. Compound 1 was administered orally (PO) on a twice a day basis (BID). The x-axis is the time measured in days and the y-axis is NCI-H1975-LUC BLI (photons/s). The experimental procedure is provided in Example 64.

FIG. 15 is a graph illustrating in vivo body weight change in female BALB/c nude mice bearing intracranial NCI-H1975-luciferase expressing tumors established by injecting tumor cells in carotid artery. Mice were treated with the vehicle control, Compound 1 at 100 mg/kg. Compound 1 was administered orally (PO) on a twice a day basis (BID). The x-axis is the time measured in days and the y-axis is % change of bodyweight. The experimental procedure is provided in Example 64.

FIG. 16 is a graph showing the probability of survival of female BALB/c nude mice bearing intracranial NCI-H1975-luciferase expressing tumors established by injecting tumor cells in carotid artery. Mice were treated with the vehicle control, Compound 1 at 100 mg/kg. Compound 1 was administered orally (PO) on a twice a day basis (BID). The x-axis is the time measured in days and the y-axis is the % probability of survival. The experimental procedure is provided in Example 64.

FIG. 17 is a cocrystal structure showing the simultaneous binding of the allosteric EGFR binding portion of Compound 1 and osimertinib in different binding pockets of L858R mutant EGFR. The allosteric EGFR binding portion of Compound 1 binds close to the L858R mutation. The experimental procedure is provided in Example 66.

FIG. 18A and FIG. 18B are SPR sensorgrams of Compound 1 mixed with either 1.5 μM EGFRₐₚₒ^{L858R} (18A) or 5 μM EGFR_{Osimertinib}^{L858R} (18B) injected over immobilized Btn-CRBN-DDB1. Concentrations of Compound 1 corresponding to each sensorgram are indicated by the key. Thin black lines represent fits to a 1:1 Langmuir binding model, with best-fit parameters for each titration experiment listed in their respective plots. The experimental procedure is provided in Example 68.

FIG. 19 is a western blot showing the effect of osimertinib on Compound 1-induced EGFR-L858R degradation and the downstream signaling in H3255 (EGFR-L858R) cells. The experimental procedure is provided in Example 69.

### DETAILED DESCRIPTION OF THE INVENTION

Compounds and their uses and manufacture are provided that degrade via the ubiquitin proteasome pathway (UPP) the epidermal growth factor receptor protein (EGFR) mediated cancer that has metastasized to the brain or CNS. The present invention provides compounds of Formula I, II, III, or IV or a pharmaceutically acceptable salt thereof that include a Targeting Ligand that binds to EGFR, an E3 Ligase binding portion (typically via a cereblon subunit), and a Linker that covalently links the Targeting Ligand to the E3 Ligase binding portion. In certain embodiments the E3 Ligase binding portion is a moiety of A or A*, the Linker is a moiety of L¹ or L², and the remainder of the molecule is the EGFR Targeting Ligand portion. In certain embodiments a compound described herein degrades EGFR with a mutation or combination of mutations, for example a mutation selected from T790M, L858R, and C797S; the combination of two mutations selected from T790M, L858R, and C797S; or the combination of two mutations selected from T790M, L858R, and C797S. In certain embodiments a compound described herein is a selective degrader of L858R-T790M, L858R-T790M-C797S, L858R, and/or L858R-C797S containing EGFR mutants.

In certain embodiments, a compound described herein provides an improved efficacy and/or safety profile relative to at least one known EGFR inhibitor. For example, the degrader described herein has the efficiency of an inhibitor only protein binding moiety combined with the catalytic degradation activity of the cereblon-mediated proteasomal degradation. This provides rapid activity against the target overexpressed EGFR by an active moiety that can quickly “return to action” and repeat the catalytic function. In this way, the EGFR is quickly destroyed as done with a covalent suicide inhibitor, like osimertinib, but without at the same time destroying the active drug.

### I. Definitions

The following definitions of the general terms used in the present description apply whether the terms appear alone or in combination with other groups.

Unless otherwise stated, the following terms used in this application, including the specification and claims, have the definitions given below. It must be noted that, as used in the specification and the appended claims, the singular forms “a”, “an,” and “the” include plural referents unless the context clearly dictates otherwise.

The term “C₁₋₆-alkoxy” denotes a group of the formula —O—R′, wherein R′ is an C₁₋₆-alkyl group, particularly C₁₋₃-alkyl. Examples of C₁₋₆-alkoxy groups include methoxy, ethoxy, n-propoxy, isopropoxy, n-butoxy, isobutoxy and tert-butoxy. Particular examples are methoxy, ethoxy and isopropoxy. More particular example is methoxy.

The term “C₁₋₆-alkyl”, alone or in combination with other groups, stands for a hydrocarbon radical which may be linear or branched, with single or multiple branching, wherein the alkyl group in general comprises 1 to 6 carbon atoms, for example, methyl (Me), ethyl (Et), propyl, isopropyl (i-propyl), n-butyl, i-butyl (isobutyl), 2-butyl (sec-butyl), t-butyl (tert-butyl), isopentyl, 2-ethyl-propyl (2-methyl-propyl), 1,2-dimethyl-propyl and the like. A specific group is methyl.

The term “cyano” denotes a —C≡N group.

The term “C₃₋₈-cycloalkoxy” denotes a group of the formula —O—R′, wherein R′ is a C₃₋₈-cycloalkyl group. Examples of cycloalkoxy group include cyclopropoxy, cyclobutoxy, cyclopentyloxy, cyclohexyloxy, cycloheptyloxy and cyclooctyloxy. Particular example is cyclopropoxy.

The term “C₃₋₈-cycloalkyl” denotes a monovalent saturated monocyclic or bicyclic hydrocarbon group of 3 to 8 ring carbon atoms. Bicyclic means a ring system consisting of two saturated carbocycles having one or two carbon atoms in common. Examples of monocyclic C₃₋₈-cycloalkyl are cyclopropyl, cyclobutanyl, cyclopentyl, cyclohexyl or cycloheptyl. Example of bicyclic C₃₋₈-cycloalkyl is spiro[3.3]heptanyl. Particular monocyclic C₃₋₈-cycloalkyl groups are cyclopropyl, cyclobutanyl. More particular monocyclic C₃₋₈-cycloalkyl groups include cyclopropyl.

The term “halo-C₁₋₆-alkoxy” denotes an C₁₋₆-alkoxy group wherein at least one of the hydrogen atoms of the C₁₋₆-alkoxy group has been replaced by same or different halogen atoms. The term “perhalo-C₁₋₆-alkoxy” denotes an C₁₋₆-alkoxy group where all hydrogen atoms of the C₁₋₆-alkoxy group have been replaced by the same or different halogen atoms. Examples of halo-C₁₋₆-alkoxy include fluoromethoxy, difluoromethoxy, trifluoromethoxy, fluoroethoxy, difluoroethoxy, trifluoroethoxy, trifluoromethylethoxy, trifluorodimethylethoxy and pentafluoroethoxy. Particular halo-C₁₋₆-alkoxy groups include fluoromethoxy, rifluoroethoxy, difluoromethoxy, difluoroethoxy, trifluoromethoxy, trifluoromethylethoxy and trifluorodimethylethoxy. More particular examples are fluoromethoxy, difluoromethoxy and trifluoromethoxy.

The term “halo-C₁₋₆-alkyl” denotes an C₁₋₆-alkyl group wherein at least one of the hydrogen atoms of the C₁₋₆-alkyl group has been replaced by the same or different halogen atoms. The term “perhalo-C₁₋₆-alkyl-C₁₋₆-alkyl” denotes an-C₁₋₆-alkyl-C₁₋₆-alkyl group where all hydrogen atoms of the alkyl group have been replaced by the same or different halogen atoms. Examples of halo-C₁₋₆-alkyl include fluoromethyl, difluoromethyl, trifluoromethyl, trifluoroethyl, trifluoromethylethyl and pentafluoroethyl. Particular halo-C₁₋₆-alkyl groups include fluoromethyl, difluoromethyl, trifluoromethyl, fluoroethyl, trifluoroethyl and difluoroethyl. More particular halo-C₁₋₆-alkyl groups include fluoromethyl.

The term “halo-C₃₋₈-cycloalkoxy” denotes an C₃₋₈-cycloalkoxy group wherein at least one of the hydrogen atoms of the C₃₋₈-cycloalkoxy group has been replaced by same or different halogen atoms. The term “perhalo-C₃₋₈-cycloalkoxy” denotes an C₃₋₈-cycloalkoxy group where all hydrogen atoms of the C₃₋₈-cycloalkoxy group have been replaced by the same or different halogen atoms. Examples of halo-C₃₋₈-cycloalkoxy include fluorocyclopropoxy, fluorocyclobutoxy, fluorocyclopentyloxy, fluorocyclohexyloxy, fluorocycloheptyloxy, difluorocyclopropoxy, difluorocyclobutoxy, difluorocyclopentyloxy, difluorocyclohexyloxy and difluorocycloheptyloxy.

The term “halo-C₃₋₈-cycloalkyl” denotes an C₃₋₈-cycloalkyl group wherein at least one of the hydrogen atoms of the C₃₋₈-cycloalkyl group has been replaced by the same or different halogen atoms. The term “perhalo-C₃₋₈-cycloalkyl” denotes an-C₃₋₈-cycloalkyl group where all hydrogen atoms of the alkyl group have been replaced by the same or different halogen atoms. Examples of halo-C₃₋₈-cycloalkyl include fluorocyclopropyl, fluorocyclobutanyl, fluorocyclopentyl, fluorocyclohexyl, fluorocycloheptyl, difluorocyclopropyl, difluorocyclobutanyl, difluorocyclopentyl, difluorocyclohexyl or difluorocycloheptyl.

The term “halogen”, alone or in combination with other groups, denotes chloro (Cl), iodo (I), fluoro (F) and bromo (Br). Specific groups are F and Cl.

The term “hydroxy” denotes a —OH group.

The term “hydroxy-C₁₋₆-alkyl alkyl” denotes an C₁₋₆-alkyl alkyl group wherein at least one of the hydrogen atoms of the C₁₋₆-alkyl alkyl group has been replaced by a hydroxy group. Examples of hydroxy-C₁₋₆-alkyl include hydroxymethyl, hydroxyethyl and hydroxypropyl. Particular example is hydroxymentyl.

The term “pharmaceutically acceptable” denotes an attribute of a material which is useful in preparing a pharmaceutical composition that is generally safe, non-toxic, and neither biologically nor otherwise undesirable and is acceptable for veterinary as well as human pharmaceutical use.

The term “a pharmaceutically acceptable salt” refers to a salt that is suitable for use in contact with the tissues of humans and animals. Examples of suitable salts with inorganic and organic acids are, but are not limited to acetic acid, citric acid, formic acid, fumaric acid, hydrochloric acid, lactic acid, maleic acid, malic acid, methane-sulfonic acid, nitric acid, phosphoric acid, p-toluenesulphonic acid, succinic acid, sulfuric acid (sulphuric acid), tartaric acid, trifluoroacetic acid and the like. Particular acids are formic acid, trifluoroacetic acid and hydrochloric acid. A specific acid is trifluoroacetic acid.

The terms “pharmaceutically acceptable auxiliary substance” refer to carriers and auxiliary substances such as diluents or excipients that are compatible with the other ingredients of the formulation.

The term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The term “as defined herein” and “as described herein” when referring to a variable incorporates by reference the broad definition of the variable as well as particularly, more particularly and most particularly definitions, if any.

The terms “treating”, “contacting” and “reacting” when referring to a chemical reaction means adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “pharmaceutically acceptable excipient” denotes any ingredient having no therapeutic activity and being non-toxic such as disintegrators, binders, fillers, solvents, buffers, tonicity agents, stabilizers, antioxidants, surfactants or lubricants used in formulating pharmaceutical products.

The term “pharmaceutical composition” encompasses a product comprising specified ingredients in pre-determined amounts or proportions, as well as any product that results, directly or indirectly, from combining specified ingredients in specified amounts. Particularly it encompasses a product comprising one or more active ingredients, and an optional carrier comprising inert ingredients, as well as any product that results, directly or indirectly, from combination, complexation or aggregation of any two or more of the ingredients, or from dissociation of one or more of the ingredients, or from other types of reactions or interactions of one or more of the ingredients.

The term “inhibitor” denotes a compound which competes with, reduces or prevents the binding of a particular ligand to particular receptor or which reduces or prevents the function of a particular protein.

The term “half maximal inhibitory concentration” (IC₅₀) denotes the concentration of a particular compound required for obtaining 50% inhibition of a biological process in vitro. IC₅₀ values can be converted logarithmically to pIC₅₀ values (−log IC₅₀), in which higher values indicate exponentially greater potency. The IC₅₀ value is not an absolute value but depends on experimental conditions e.g. concentrations employed. The IC₅₀ value can be converted to an absolute inhibition constant (Kᵢ) using the Cheng-Prusoff equation (Biochem. Pharmacol. (1973) 22:3099).

“Therapeutically effective amount” means an amount of a compound that, when administered to a subject for treating a disease state, is sufficient to effect such treatment for the disease state. The “therapeutically effective amount” will vary depending on the compound, disease state being treated, the severity or the disease treated, the age and relative health of the subject, the route and form of administration, the judgment of the attending medical or veterinary practitioner, and other factors.

The term “aromatic” denotes the conventional idea of aromaticity as defined in the literature, in particular in IUPAC—Compendium of Chemical Terminology, 2nd, A. D. McNaught & A. Wilkinson (Eds). Blackwell Scientific Publications, Oxford (1997).

Whenever a chiral carbon is present in a chemical structure, it is intended that all stereoisomers associated with that chiral carbon are encompassed by the structure as pure stereoisomers as well as mixtures thereof.

In certain embodiments, isotopes are incorporated into the compounds of the invention. These isotopes include, but are not limited to, isotopes of hydrogen, carbon, nitrogen, oxygen, fluorine, and chlorine such as ²H, ³H, ¹¹C, ¹³C, ¹⁴C, ¹⁵N, ¹⁷O, ¹⁸O, ¹⁸F, ³⁵S, and ³⁶Cl respectively. In one non-limiting embodiment, isotopically labelled compounds can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Additionally, any hydrogen atom present in the compound of the invention may be substituted with an ¹⁸F atom, a substitution that may be particularly desirable for PET or SPECT studies.

In one non-limiting embodiment, the substitution of a hydrogen atom for a deuterium atom can be provided in any compound described herein. For example, when any of the groups are, or contain for example through substitution, methyl, ethyl, or methoxy, the alkyl residue may be deuterated (in non-limiting embodiments, CDH₂, CD₂H, CD₃, CH₂CD₃, CD₂CD₃, CHDCH₂D, CH₂CD₃, CHDCHD₂, OCDH₂, OCD₂H, or OCD₃ etc.). In certain other embodiments, when two substituents are combined to form a cycle the unsubstituted carbons may be deuterated. In certain embodiments, at least one deuterium is placed on an atom that has a bond which is broken during metabolism of the compound in vivo, or is one, two or three atoms remote form the metabolized bond (e.g., which may be referred to as an α, β or γ, or primary, secondary or tertiary isotope effect).

In certain embodiments a compound described herein is isotopically labeled. In certain embodiments at least one R group independently selected from R¹, R², R³, R⁴, R⁶, R⁷, R⁸, R⁹, R¹⁰, R¹¹, R¹², R¹³, R¹⁴, R¹⁵, R¹⁶, R¹⁷, R²⁰, R²¹, R²², R²³, R²⁴, R²⁶, R²⁷, R³¹, R³², R³³, R³⁴, R³⁵, R³⁶, R³⁷, R⁴⁰, R⁴¹, R⁴², R⁵², R⁵³, R⁵⁴, R⁵⁵, R⁷⁰, R⁸⁰, R⁸¹, R⁸², R⁹⁰, or R¹⁰⁰ is isotopically labeled with 1, 2, or more isotopes as allowed by valence. In certain embodiments the isotopic label is deuterium. In certain embodiments, at least one deuterium is placed on an atom that has a bond which is broken during metabolism of the compound in vivo, or is one, two or three atoms remote form the metabolized bond (e.g., which may be referred to as an α, β or γ, or primary, secondary or tertiary isotope effect). In another embodiment the isotopic label is ¹³C. In other embodiments the isotopic label is ¹⁸F.

In certain embodiments the compounds described herein may form a solvate with a solvent (including water). Therefore, in one non-limiting embodiment, the invention includes a solvated form of the compounds described herein. The term “solvate” refers to a molecular complex of a compound described herein (including a salt thereof) with one or more solvent molecules. Non-limiting examples of solvents are water, ethanol, isopropanol, dimethyl sulfoxide, acetone and other common organic solvents.

In certain embodiments “alkenyl” is a linear or branched aliphatic hydrocarbon groups having one or more carbon-carbon double bonds that may occur at a stable point along the chain. In one non-limiting embodiment, the alkenyl contains from 2 to about 12 carbon atoms, more generally from 2 to about 6 carbon atoms or from 2 to about 4 carbon atoms. In certain embodiments the alkenyl is C₂, C₂-C₃, C₂-C₄, C₂-C₅, or C₂-C₆. In certain embodiments, examples of alkenyl radicals include, but are not limited to ethenyl, propenyl, allyl, propenyl, butenyl and 4-methylbutenyl. In certain embodiments the term “alkenyl” also embodies “cis” and “trans” alkenyl geometry, or alternatively, “E” and “Z” alkenyl geometry. In certain embodiments the term “alkenyl” also encompasses cycloalkyl or carbocyclic groups having at least one point of unsaturation.

In certain embodiments “alkynyl” is a branched or straight chain aliphatic hydrocarbon group having one or more carbon-carbon triple bonds that may occur at any stable point along the chain. In one non-limiting embodiment, the alkynyl contains from 2 to about 12 carbon atoms, more generally from 2 to about 6 carbon atoms or from 2 to about 4 carbon atoms. In certain embodiments the alkynyl is C₂, C₂-C₃, C₂-C₄, C₂-C₅, or C₂-C₆. In certain embodiments, examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl. In certain embodiments, the term “alkynyl” also encompasses cycloalkyl or carbocyclic groups having at least one point of triple bond unsaturation.

In certain embodiments the term “CNS” refers to a component of the central nervous system including, for example, the brain, brain stem, peripheral nervous system, cerebral spinal fluid, spinal cord, leptomeninges, epidural space, myelin, thalamus, hypothalamus, pituitary gland, hippocampus, cerebellum, cerebrum, midbrain, pons, frontal lobe, temporal lobe, and/or dura.

### II. Methods of Treating EGFR Mediated Disorders with Compounds of Formula I, II, III, and IV

The invention provides methods of using compounds of Formulas I, II, III, and IV.
E1: In certain embodiments the invention is a method of treating a patient with an EGFR mediated cancer that has metastasized to the brain, central nervous system, peripheral nervous system, cerebral spinal fluid, spinal cord, leptomeninges, epidural space, and/or dura comprising administering an effective amount of an EGFR degrading compound of Formula:

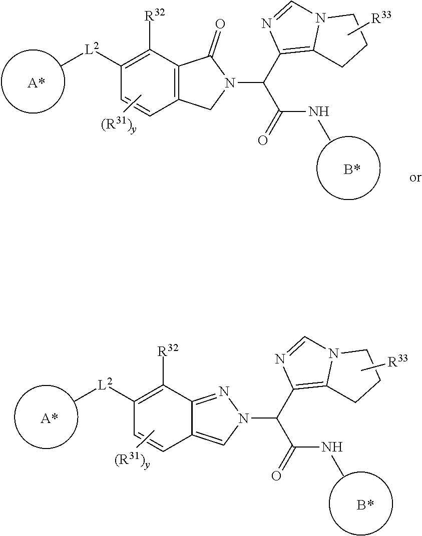

or a pharmaceutically acceptable salt, isotope, N-oxide, stereoisomer thereof, optionally as part of a pharmaceutical composition, to a patient in need thereof;
wherein
   A* is selected from:

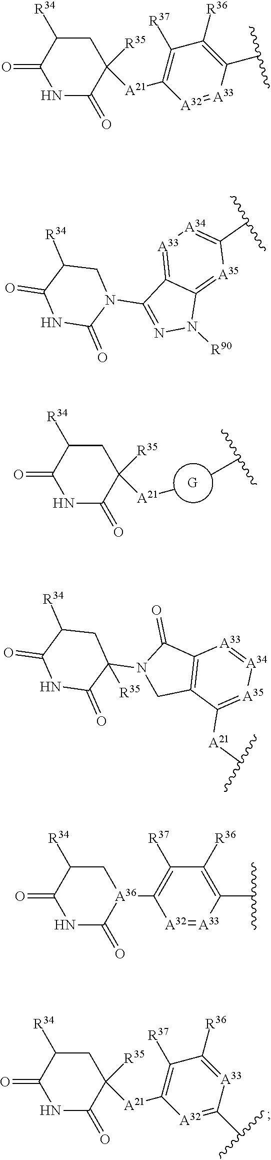

B* is heteroaryl or aryl each of which is optionally substituted with 1, 2, or 3 R³¹ substituents;
y is 0, 1, 2, or 3;
R³¹ is independently selected at each occurrence from H, halogen (F, Cl, Br, or I), C₁₋₆-alkyl, cyano, C₁₋₆-alkoxy, halo-C₁₋₆-alkoxy, halo-C₁₋₆-alkyl, C₃₋₈-cycloalkyl, and halo-C₃₋₈-cycloalkyl and can be located on either ring where present on a bicycle;
R³² is hydrogen, halogen (F, Cl, Br, or I), C₁₋₆-alkyl, halo-C₁₋₆-alkyl, C₃₋₈-cycloalkyl, or halo-C₃₋₈-cycloalkyl;
R³³ is hydrogen, halogen (F, Cl, Br, or I), C₁₋₆-alkyl, halo-C₁₋₆-alkyl, C₃₋₈-cycloalkyl, or halo-C₃₋₈-cycloalkyl and can be located on the dihydropyrrole or imidazole ring;
R³⁴ is independently selected at each occurrence from H, F, C₁₋₆-alkyl, halo-C₁₋₆-alkyl, C₃₋₈-cycloalkyl, and halo-C₃₋₈-cycloalkyl;
R³⁵ is independently selected at each occurrence from H, halogen (F, Cl, Br, or I), C₁₋₆-alkyl, halo-C₁₋₆-alkyl, and C₃₋₈-cycloalkyl;
or R³⁴ and R³⁵ combine to form —(CH₂)_{q}—;
q is 1 or 2;
R³⁶ and R³⁷ are independently selected from H, halogen (F, Cl, Br, or I), cyano, C₁₋₆-alkoxy, halo-C₁₋₆-alkoxy, C₁₋₆-alkyl, halo-C₁₋₆-alkyl, C₃₋₈-cycloalkyl, and halo-C₃₋₈-cycloalkyl;
or R³⁶ and R³⁷ together are combined to form a 5- or 6-membered cycle optionally substituted with 1, 2, or 3 R³¹ substituents;
R⁹⁰ is H, C₁₋₆-alkyl, or C₃₋₆-cycloalkyl;
Ring G is a heteroaryl optionally substituted with 1 or 2 R⁴² substituents;
A²¹ is —NH—, —O—, —CH₂—, or —NR¹⁰⁰—;
R¹⁰⁰ is alkyl, cycloalkyl, aryl, or heteroaryl; or as allowed by valence R¹⁰⁰ may combine with R³⁷ to form a 5-8 membered heterocycle or 5 membered heteroaryl;
A³², A³³, A³⁴, and A³⁵ are independently selected from —N— and —CR⁴²—;
R⁴² is independently selected at each occurrence from H, halogen (F, Cl, Br, or I), cyano, C₁₋₆-alkoxy, halo-C₁₋₆-alkoxy, C₁₋₆-alkyl, halo-C₁₋₆-alkyl, C₃₋₈-cycloalkyl, and halo-C₃₋₈-cycloalkyl;
A³⁶ is —N— or —CR³⁵—;
L² is a bivalent linking group that connects A* and either the isoindolinone or indazole.
E2: The method of embodiment 1, wherein the EGFR degrading compound is selected from:

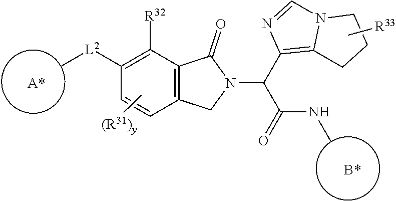

or a pharmaceutically acceptable salt thereof.
E3: The method of embodiment 1, wherein the EGFR degrading compound is selected from:

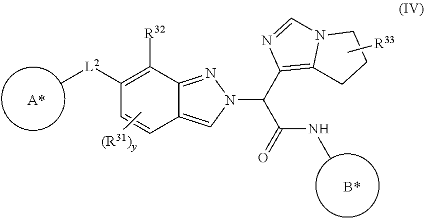

or a pharmaceutically acceptable salt thereof.
E4: The method of any one of embodiments 1-3, wherein R³³ is H.
E5: The method of any one of embodiments 1-3, wherein R³³ is F.
E6: The method of any one of embodiments 1-5, wherein y is 1.
   E7: The method of any one of embodiments 1-5, wherein y is 2.
E8: The method of any one of embodiments 1-7, wherein at least one R³¹ is halo.
E9: The method of any one of embodiments 1-7, wherein at least one R³¹ is F.
E10: The method of any one of embodiments 1-3, wherein y is 0.
E11: The method of any one of embodiments 1-9, wherein R³² is H.
E12: The method of any one of embodiments 1-9, wherein R³² is F.
E13: The method of embodiment 1, wherein the EGFR degrading compound is selected from:

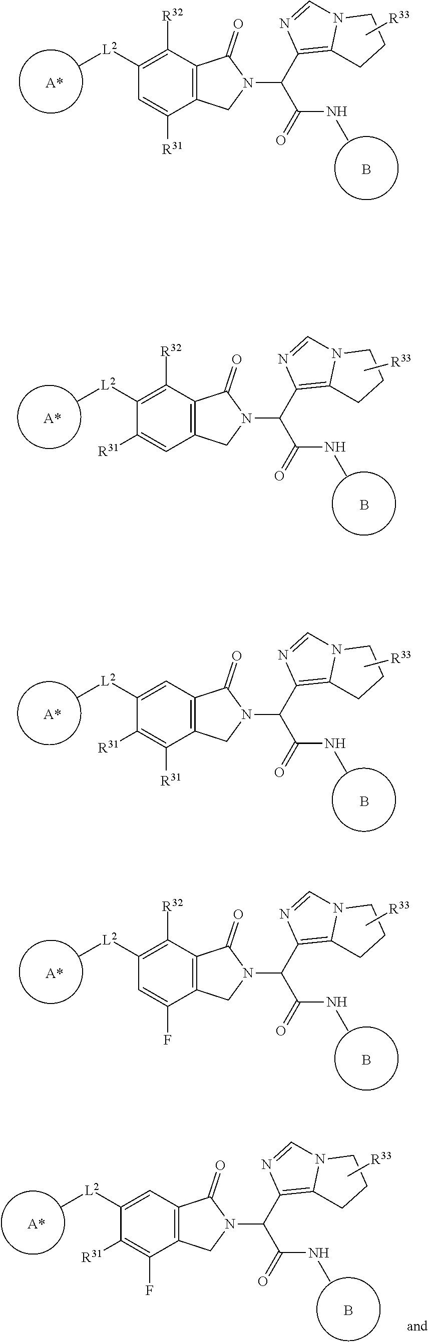

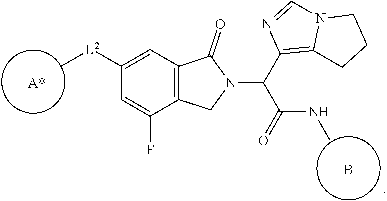

E14: The method of embodiment 1, wherein the EGFR degrading compound is selected from:

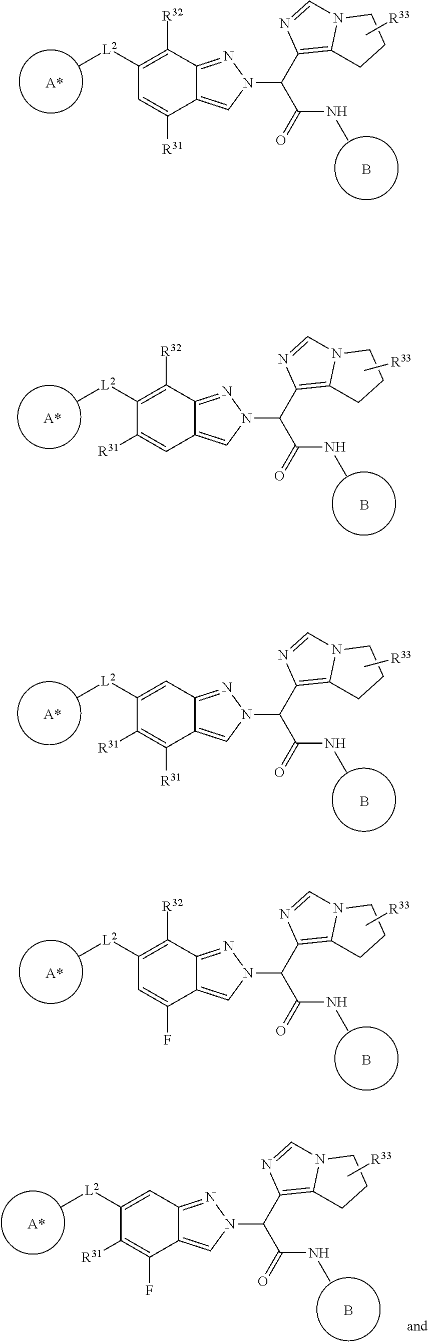

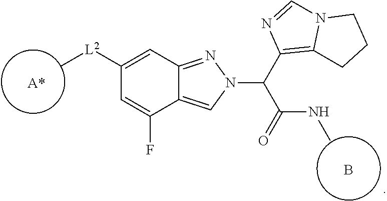

E15: The method of any one of embodiments 1-14, wherein A* is:

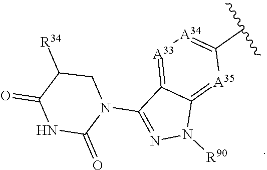

E16: The method of any one of embodiments 1-14, wherein A* is:

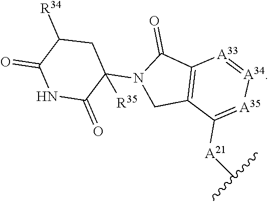

E17: The method of any one of embodiments 1-16, wherein A³⁴ is CH.
E18: The method of any one of embodiments 1-16, wherein A³⁴ is N.
E19: The method of any one of embodiments 1-16, wherein A³⁴ is CR⁴².
E20: The method of any one of embodiments 1-16, wherein A³⁴ is CF.
E21: The method of any one of embodiments 1-20, wherein A³⁵ is CH.
E22: The method of any one of embodiments 1-20, wherein A³⁵ is N.
E23: The method of any one of embodiments 1-20, wherein A³⁵ is CR⁴².
E24: The method of any one of embodiments 1-20, wherein A³⁵ is CF.
E25: The method of any one of embodiments 1-14, wherein A* is:

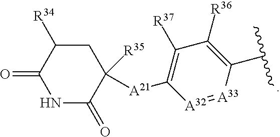

E26: The method of any one of embodiments 1-14, wherein A* is:

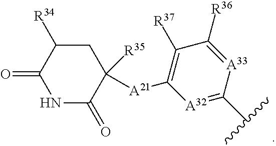

E27: The method of any one of embodiments 25 or 26, wherein A²¹ is NH.
E28: The method of any one of embodiments 25 or 26, wherein A²¹ is O.
E29: The method of any one of embodiments 1-14, wherein A* is:

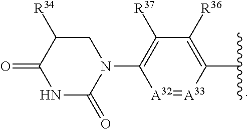

E30: The method of any one of embodiments 1-29, wherein A³² is CH.
E31: The method of any one of embodiments 1-29, wherein A³² is N.
E32: The method of any one of embodiments 1-29, wherein A³² is CR⁴².
E33: The method of any one of embodiments 1-29, wherein A³² is CF.
E34: The method of any one of embodiments 1-33, wherein A³³ is CH.
E35: The method of any one of embodiments 1-33, wherein A³³ is N.
E36: The method of any one of embodiments 1-33, wherein A³³ is CR⁴².
E37: The method of any one of embodiments 1-33, wherein A³³ is CF.
E38: The method of any one of embodiments 1-14, wherein A* is:

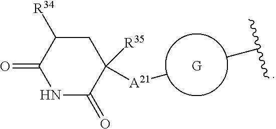

E39: The method of embodiments 38, wherein A²¹ is NH.
E40: The method of embodiments 38, wherein A²¹ is O.
E41: The method of any one of embodiments 1-40, wherein R³⁴ is H.
E42: The method of any one of embodiments 1-40, wherein R³⁴ is F.
E43: The method of any one of embodiments 1-40, wherein R³⁴ is CH₃.
E44: The method of any one of embodiments 1-43, wherein R³⁵ is H.
E45: The method of any one of embodiments 1-43, wherein R³⁵ is F.
E46: The method of any one of embodiments 1-43, wherein R³⁵ is CH₃.
E47: The method of any one of embodiments 1-40, wherein R³⁴ and R³⁵ combine to form a —CH₂—.
E48: The method of any one of embodiments 1-47, wherein R³¹ is independently selected at each instance from H, halogen (F, Cl, Br, or I), and C₁₋₆-alkyl.
E49: The method of any one of embodiments 1-47, wherein R⁴² is independently selected at each instance from H, halogen (F, Cl, Br, or I), and C₁₋₆-alkyl.
E50: The method of any one of embodiments 1-49, wherein B* is

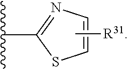

E51: The method of any one of embodiments 1-49, wherein B* is

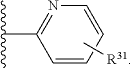

E52: The method of any one of embodiments 1-49, wherein B* is

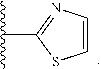 [SMILES: *c1nccs1]

E53: The method of any one of embodiments 1-49, wherein B* is

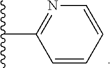 [SMILES: *c1ccccn1]

E54: The method of any one of embodiments 1-53, wherein L² is of formula:

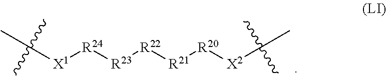

wherein,
   X¹ and X² are independently at each occurrence selected from bond, heterocycle, aryl, heteroaryl, bicycle, alkyl, aliphatic, heteroaliphatic, —NR²⁷—, —CR⁴⁰R⁴¹—, —O—, —C(O)—, —C(NR²⁷)—, —C(S)—, —S(O)—, —S(O)₂— and —S—; each of which heterocycle, aryl, heteroaryl, and bicycle is optionally substituted with 1, 2, 3, or 4 substituents independently selected from R⁴⁰;
   R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, oxyalkylene, —C(R⁴⁰R⁴⁰)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, and carbocycle; each of which is optionally substituted with 1, 2, 3, or 4 substituents independently selected from R⁴⁰;
   R²⁶ is independently at each occurrence selected from the group consisting of hydrogen, alkyl, arylalkyl, heteroarylalkyl, alkene, alkyne, aryl, heteroaryl, heterocycle, aliphatic and heteroaliphatic;
   R²⁷ is independently at each occurrence selected from the group consisting of hydrogen, alkyl, aliphatic, heteroaliphatic, heterocycle, aryl, heteroaryl, —C(O)(aliphatic, aryl, heteroaliphatic or heteroaryl), —C(O)O(aliphatic, aryl, heteroaliphatic, or heteroaryl), alkene, and alkyne;
   R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(aliphatic), —N(aliphatic)₂, —NHSO₂(aliphatic), —N(aliphatic)SO₂alkyl, —NHSO₂(aryl, heteroaryl or heterocycle), —N(alkyl)SO₂(aryl, heteroaryl or heterocycle), —NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heterocycle, oxo, and cycloalkyl; additionally, where allowed by valence two R⁴⁰ groups bound to the same carbon may be joined together to form a 3-8 membered spirocycle; and
   R⁴¹— is aliphatic, aryl, heteroaryl, or hydrogen.
E55: The method of any one of embodiments 1-54, wherein L² is of formula:

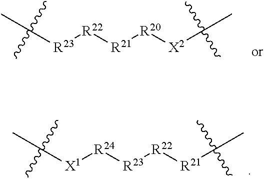

E56: The method of embodiment 54 or 55, wherein X¹ is bond.
E57: The method of embodiment 54 or 55, wherein X¹ is heterocycle.
E58: The method of embodiment 54 or 55, wherein X¹ is NR².
E59: The method of embodiment 54 or 55, wherein X¹ is C(O).
E60: The method of any one of embodiments 54 to 59, wherein X² is bond.
E61: The method of any one of embodiments 54 to 59, wherein X² is heterocycle.
E62: The method of any one of embodiments 54 to 59, wherein X² is NR².
E63: The method of any one of embodiments 54 to 59, wherein X² is C(O).
E64: The method of any one of embodiments 54 to 63, wherein R²⁰ is bond.
E65: The method of any one of embodiments 54 to 63, wherein R²⁰ is CH₂.
E66: The method of any one of embodiments 54 to 63, wherein R²⁰ is heterocycle.
E67: The method of any one of embodiments 54 to 63, wherein R²⁰ is aryl.
E68: The method of any one of embodiments 54 to 63, wherein R²⁰ is phenyl.
E69: The method of any one of embodiments 54 to 63, wherein R²⁰ is bicycle.
E70: The method of any one of embodiments 54 to 69, wherein R²¹ is bond.
E71: The method of any one of embodiments 54 to 69, wherein R²¹ is CH₂.
E72: The method of any one of embodiments 54 to 69, wherein R²¹ is heterocycle.
E73: The method of any one of embodiments 54 to 69, wherein R²¹ is aryl.
E74: The method of any one of embodiments 54 to 69, wherein R²¹ is phenyl.
E75: The method of any one of embodiments 54 to 69, wherein R²¹ is bicycle.
E76: The method of embodiment 54, wherein L is a linker of formula:

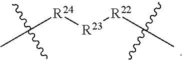

E77: The method of any one of embodiments 54 to 76, wherein R²² is bond.
E78: The method of any one of embodiments 54 to 76, wherein R²² is CH₂.
E79: The method of any one of embodiments 54 to 76, wherein R²² is heterocycle.
E80: The method of any one of embodiments 54 to 76, wherein R²² is aryl.
E81: The method of any one of embodiments 54 to 76, wherein R²² is phenyl.
E82: The method of any one of embodiments 54 to 76, wherein R²² is bicycle.
E83: The method of any one of embodiments 54 to 69, wherein L is a linker of formula:

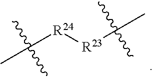

E84: The method of any one of embodiments 54 to 83, wherein R²³ is bond.
E85: The method of any one of embodiments 54 to 83, wherein R²³ is CH₂.
E86: The method of any one of embodiments 54 to 83, wherein R²³ is heterocycle.
E87: The method of any one of embodiments 54 to 83, wherein R²³ is aryl.
E88: The method of any one of embodiments 54 to 83, wherein R²³ is phenyl.
E89: The method of any one of embodiments 54 to 83, wherein R²³ is bicycle.
E90: The method of any one of embodiments 54 to 89, wherein R²⁴ is bond.
E91: The method of any one of embodiments 54 to 89, wherein R²⁴ is CH₂.
E92: The method of any one of embodiments 54 to 89, wherein R²⁴ is heterocycle.
E93: The method of any one of embodiments 54 to 89, wherein R²⁴ is aryl.
E94: The method of any one of embodiments 54 to 89, wherein R²⁴ is phenyl.
E95: The method of any one of embodiments 54 to 89, wherein R²⁴ is bicycle.
E96: The method of any one of embodiments 54 to 89, wherein R²⁴ is C(O).
E97: The method of any one of embodiments 1-96, wherein the patient is a human.
E98: The method of any one of embodiments 1-97, wherein the cancer is lung cancer.
E99: The method of embodiment 98, wherein the lung cancer is non-small cell lung cancer.
E100: The method of any one of embodiments 1-99, wherein the cancer has an EGFR protein with at least one mutation.
E101: The method of any one of embodiments 1-100, wherein the cancer has an EGFR protein with the L858R mutation.
E102: The method of any one of embodiments 1-101, wherein the cancer has an EGFR protein with the T790M mutation.
E103: The method of any one of embodiments 1-102, wherein the cancer has an EGFR protein with the C797S mutation.
E104: The method of any one of embodiments 1-103, wherein the cancer has an EGFR protein with the L792H mutation.
E105: The method of any one of embodiments 1-104, wherein the cancer has an EGFR protein with the L718Q mutation.
E106: The method of any one of embodiments 1-105, wherein the cancer has an EGFR protein with the L858R-T790M mutation.
E107: The method of any one of embodiments 1-106, wherein the cancer has an EGFR protein with the L858R-T790M-C797S mutation.
E108: The method of any one of embodiments 1-107, wherein the cancer has an EGFR protein with the L858R-C797S mutation.
E109: The method of any one of embodiments 1-108, wherein an additional EGFR inhibitor is administered.
E110: The method of embodiment 109, wherein the additional EGFR inhibitor is a tyrosine kinase inhibitor.
E111: The method of embodiment 109, wherein the additional EGFR inhibitor is osimertinib.
E112: The method of embodiment 109, wherein the additional EGFR inhibitor is rociletinib.
E113: The method of embodiment 109, wherein the additional EGFR inhibitor is avitinib.
E114: The method of embodiment 109, wherein the additional EGFR inhibitor is lazertinib.
E115: The method of embodiment 109, wherein the additional EGFR inhibitor is nazartinib.
E116: The method of embodiment 109, wherein the additional EGFR inhibitor is an antibody to a mutated form of EGFR.
E117: The method of embodiment 109, wherein the additional EGFR inhibitor is cetuximab.
E118: The method of embodiment 109, wherein the additional EGFR inhibitor is panitumab.
E119: The method of embodiment 109, wherein the additional EGFR inhibitor is necitumab.
E120: The method of any one of embodiments 1-119, wherein a MET inhibitor is also administered.
E121: The method of any one of embodiments 1-120, wherein the patient receives an additional chemotherapeutic agent.
E122: The method of any one of embodiments 1-121, wherein the EGFR degrading compound is:

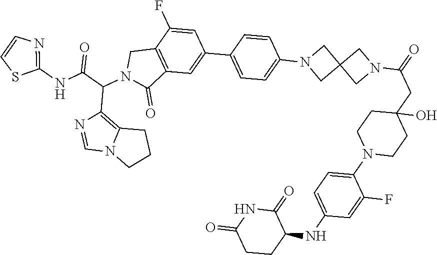 [SMILES: O=C1CC[C@H](Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1]

or a pharmaceutically acceptable salt thereof.
E123: The method of any one of embodiments 1-121, wherein the EGFR degrading compound is:

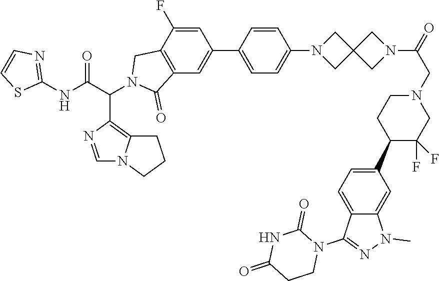 [SMILES: Cn1nc(N2CCC(=O)NC2=O)c2ccc([C@H]3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21]

or a pharmaceutically acceptable salt thereof
E124: The method of any one of embodiments 1-121, wherein the EGFR degrading

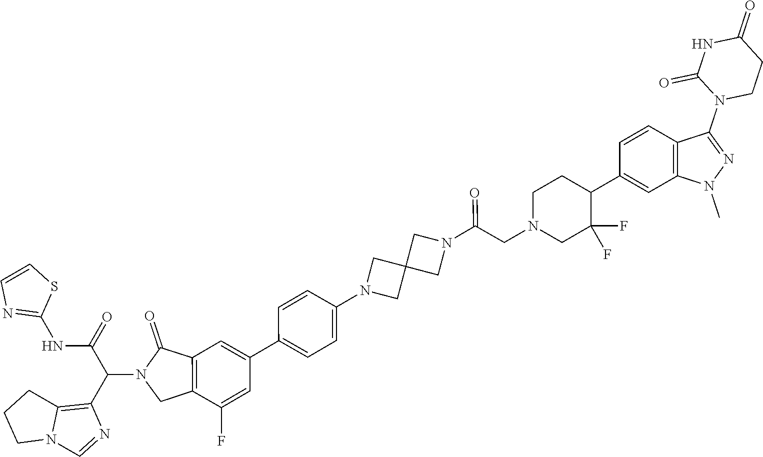 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21]

or a pharmaceutically acceptable salt thereof
E125: The method of any one of embodiments 1-121, wherein the EGFR degrading compound is:

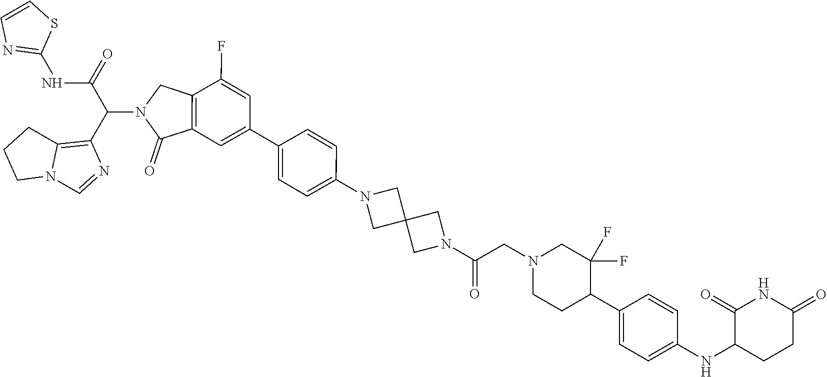 [SMILES: O=C1CCC(Nc2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc2)C(=O)N1]

or a pharmaceutically acceptable salt thereof
E126: The method of any one of embodiments 1-121, wherein the EGFR degrading

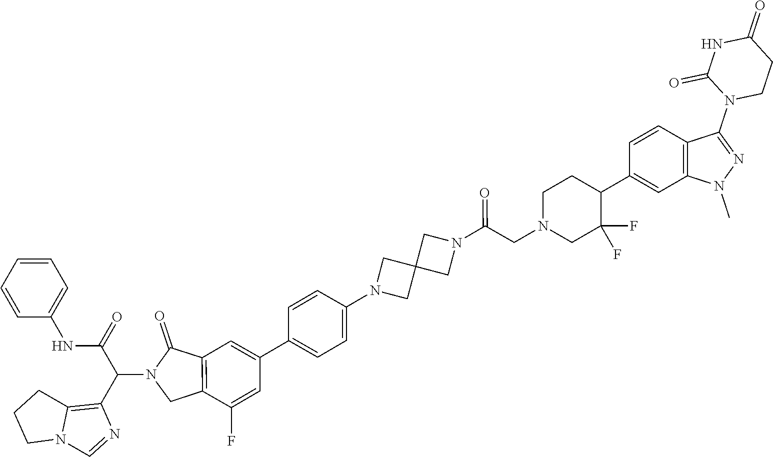 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6ccccc6)c6ncn8c6CCC8)C7)cc4)C5)CC3(F)F)cc21]

or a pharmaceutically acceptable salt thereof
E127: The method of any one of embodiments 1-121, wherein the EGFR degrading compound is:

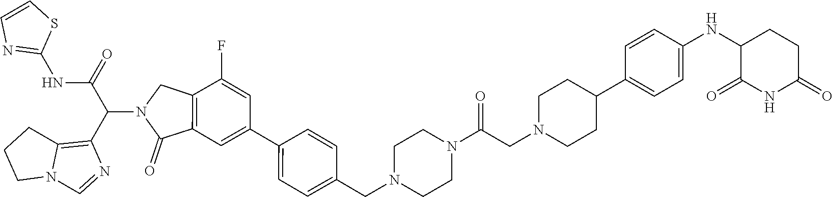 [SMILES: O=C1CCC(Nc2ccc(C3CCN(CC(=O)N4CCN(Cc5ccc(-c6cc(F)c7c(c6)C(=O)N(C(C(=O)Nc6nccs6)c6ncn8c6CCC8)C7)cc5)CC4)CC3)cc2)C(=O)N1]

or a pharmaceutically acceptable salt thereof.
E128: The method of any one of embodiments 1-121, wherein the EGFR degrading compound is:

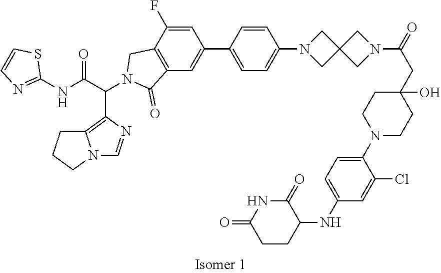 [SMILES: O=C1CCC(Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1]

or a pharmaceutically acceptable salt thereof
E129: The method of any one of embodiments 1-121, wherein the EGFR degrading

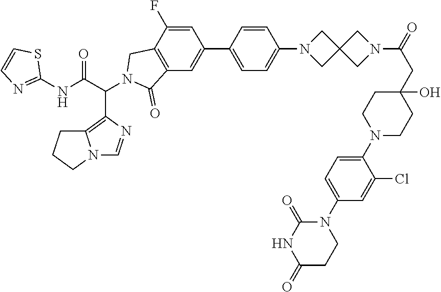 [SMILES: O=C1CCN(c2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1]

or a pharmaceutically acceptable salt thereof
E130: The method of any one of embodiments 1-121, wherein the EGFR degrading compound is:

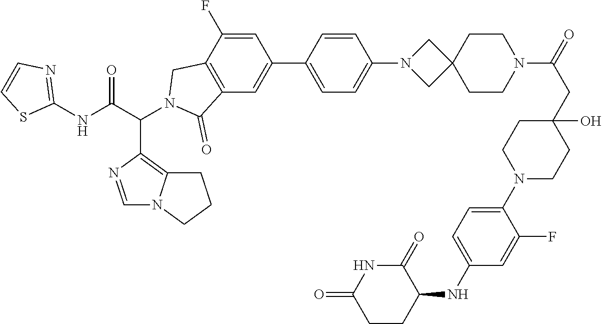 [SMILES: O=C1CC[C@H](Nc2ccc(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1]

or a pharmaceutically acceptable salt thereof
E131: The method of any one of embodiments 1-121, wherein the EGFR degrading

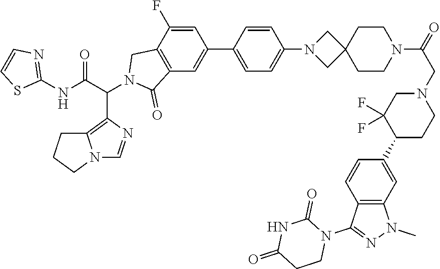 [SMILES: Cn1nc(N2CCC(=O)NC2=O)c2ccc([C@H]3CCN(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3(F)F)cc21]

or a pharmaceutically acceptable salt thereof
E132: The method of any one of embodiments 1-121, wherein the EGFR degrading compound is:

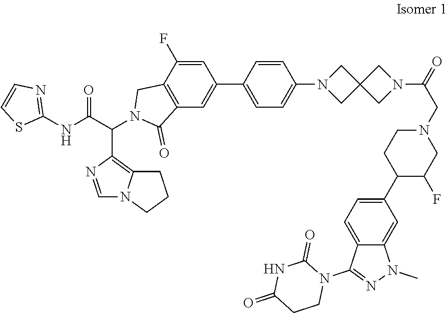 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3F)cc21]

or a pharmaceutically acceptable salt thereof
E133: The method of any one of embodiments 1-121, wherein the EGFR degrading

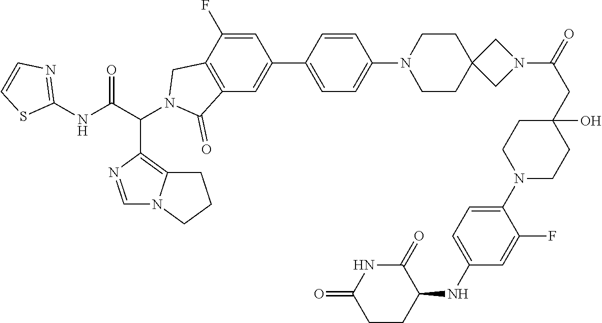 [SMILES: O=C1CC[C@H](Nc2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1]

or a pharmaceutically acceptable salt thereof
E134: The method of any one of embodiments 1-121, wherein the EGFR degrading compound is described herein.
E135: The method of any one of embodiments 1-121, wherein the EGFR degrading compound is described in Table 8, Table 9A, or Table 9B.
   1. In certain embodiments a method of treating an EGFR mediated cancer that has metastasized to the brain or CNS comprising administering an effective amount of a Compound selected from:

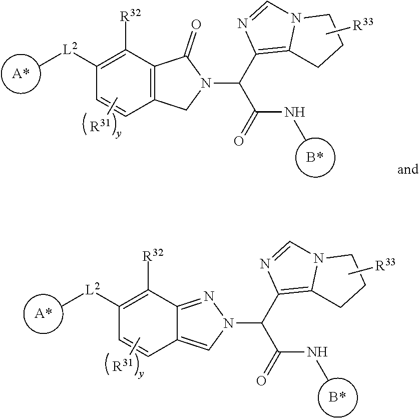

or a pharmaceutically acceptable salt thereof to a patient in need thereof is provided;
wherein
   A* is selected from:

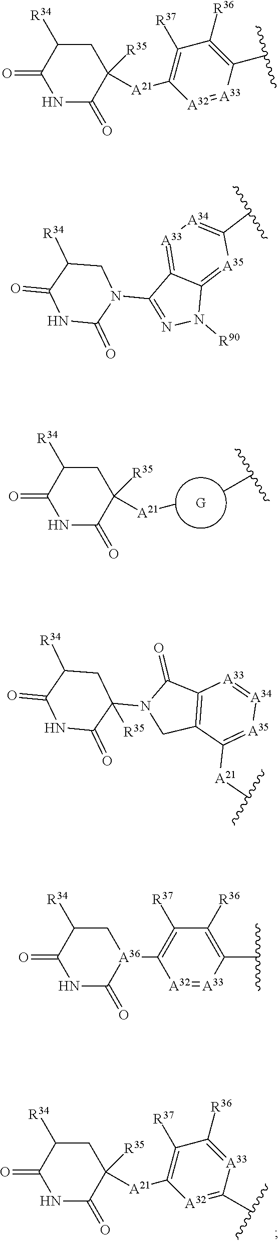

B* is heteroaryl or aryl each of which is optionally substituted with 1, 2, or 3 R³¹ substituents;
y is 0, 1, 2, or 3;
R³¹ is independently selected at each occurrence from H, halogen (F, Cl, Br, or I), C₁₋₆-alkyl, cyano, C₁₋₆-alkoxy, halo-C₁₋₆-alkoxy, halo-C₁₋₆-alkyl, C₃₋₈-cycloalkyl, and halo-C₃₋₈-cycloalkyl and can be located on either ring where present on a bicycle;
R³² is hydrogen, halogen (F, Cl, Br, or I), C₁₋₆-alkyl, halo-C₁₋₆-alkyl, C₃₋₈-cycloalkyl, or halo-C₃₋₈-cycloalkyl;
R³³ is hydrogen, halogen (F, Cl, Br, or I), C₁₋₆-alkyl, halo-C₁₋₆-alkyl, C₃₋₈-cycloalkyl, or halo-C₃₋₈-cycloalkyl and can be located on the dihydropyrrole or imidazole ring;
R³⁴ is independently selected at each occurrence from H, F, C₁₋₆-alkyl, halo-C₁₋₆-alkyl, C₃₋₈-cycloalkyl, and halo-C₃₋₈-cycloalkyl;
R³⁵ is independently selected at each occurrence from H, halogen (F, Cl, Br, or I), C₁₋₆-alkyl, halo-C₁₋₆-alkyl, and C₃₋₈-cycloalkyl;
or R³⁴ and R³⁵ combine to form —(CH₂)_{q}—;
q is 1 or 2; R³⁶ and R³⁷ are independently selected from H, halogen (F, Cl, Br, or I), cyano, C₁₋₆-alkoxy, halo-C₁₋₆-alkoxy, C₁₋₆-alkyl, halo-C₁₋₆-alkyl, C₃₋₈-cycloalkyl, and halo-C₃₋₈-cycloalkyl;
or R³⁶ and R³⁷ together are combined to form a 5- or 6-membered cycle optionally substituted with 1, 2, or 3 R³¹ substituents;
R⁹⁰ is H, C₁₋₆-alkyl, or C₃₋₆-cycloalkyl;
Ring G is a heteroaryl optionally substituted with 1 or 2 R⁴² substituents;
A²¹ is —NH—, —O—, —CH₂—, or —NR¹⁰⁰—;
R¹⁰⁰ is alkyl, cycloalkyl, aryl, or heteroaryl; or as allowed by valence R¹⁰⁰ may combine with R³⁷ to form a 5-8 membered heterocycle or 5 membered heteroaryl;
A³², A³³, A³⁴, and A³⁵ are independently selected from —N— and —CR⁴²—;
R⁴² is independently selected at each occurrence from H, halogen (F, Cl, Br, or I), cyano, C₁₋₆-alkoxy, halo-C₁₋₆-alkoxy, C₁₋₆-alkyl, halo-C₁₋₆-alkyl, C₃₋₈-cycloalkyl, and halo-C₃₋₈-cycloalkyl;
A³⁶ is —N— or —CR³⁵—;
L² is a bivalent linking group that connects A* and either the isoindolinone or indazole.
2. The method of embodiment 1, wherein L² is of formula:

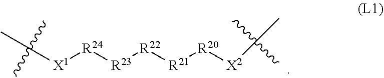

wherein,
   X¹ and X² are independently at each occurrence selected from bond, heterocycle, aryl, heteroaryl, bicycle, alkyl, aliphatic, heteroaliphatic, —NR²⁷—, —CR⁴⁰R⁴¹—, —O—, —C(O)—, —C(NR²⁷)—, —C(S)—, —S(O)—, —S(O)₂—and —S—; each of which heterocycle, aryl, heteroaryl, and bicycle is optionally substituted with 1, 2, 3, or 4 substituents independently selected from R⁴⁰;
   R²⁰, R²¹, R²², R²³, and R²⁴ are independently at each occurrence selected from the group consisting of a bond, alkyl, —C(O)—, —C(O)O—, —OC(O)—, —SO₂—, —S(O)—, —C(S)—, —C(O)NR²⁷—, —NR²⁷C(O)—, —O—, —S—, —NR²⁷—, oxyalkylene, —C(R⁴⁰R⁴⁰)—, —P(O)(OR²⁶)O—, —P(O)(OR²⁶)—, bicycle, alkene, alkyne, haloalkyl, alkoxy, aryl, heterocycle, aliphatic, heteroaliphatic, heteroaryl, lactic acid, glycolic acid, and carbocycle; each of which is optionally substituted with 1, 2, 3, or 4 substituents independently selected from R⁴⁰;
   R²⁶ is independently at each occurrence selected from the group consisting of hydrogen, alkyl, arylalkyl, heteroarylalkyl, alkene, alkyne, aryl, heteroaryl, heterocycle, aliphatic and heteroaliphatic;
   R²⁷ is independently at each occurrence selected from the group consisting of hydrogen, alkyl, aliphatic, heteroaliphatic, heterocycle, aryl, heteroaryl, —C(O)(aliphatic, aryl, heteroaliphatic or heteroaryl), —C(O)O(aliphatic, aryl, heteroaliphatic, or heteroaryl), alkene, and alkyne;
   R⁴⁰ is independently at each occurrence selected from the group consisting of hydrogen, R²⁷, alkyl, alkene, alkyne, fluoro, bromo, chloro, hydroxyl, alkoxy, azide, amino, cyano, —NH(aliphatic), —N(aliphatic)₂, —NHSO₂(aliphatic), —N(aliphatic)SO₂alkyl, —NHSO₂(aryl, heteroaryl or heterocycle), —N(alkyl)SO₂(aryl, heteroaryl or heterocycle), —NHSO₂alkenyl, —N(alkyl)SO₂alkenyl, —NHSO₂alkynyl, —N(alkyl)SO₂alkynyl, haloalkyl, aliphatic, heteroaliphatic, aryl, heteroaryl, heterocycle, oxo, and cycloalkyl; additionally, where allowed by valence two R⁴⁰ groups bound to the same carbon may be joined together to form a 3-8 membered spirocycle; and
   R⁴¹ is aliphatic, aryl, heteroaryl, or hydrogen.
   3. The method of embodiment 1, wherein the Compound is selected from Table 9A and Table 9B.
   4. In certain embodiments a method of treating an EGFR mediated cancer that has metastasized to the brain or CNS comprising administering an effective amount of a Compound selected from:

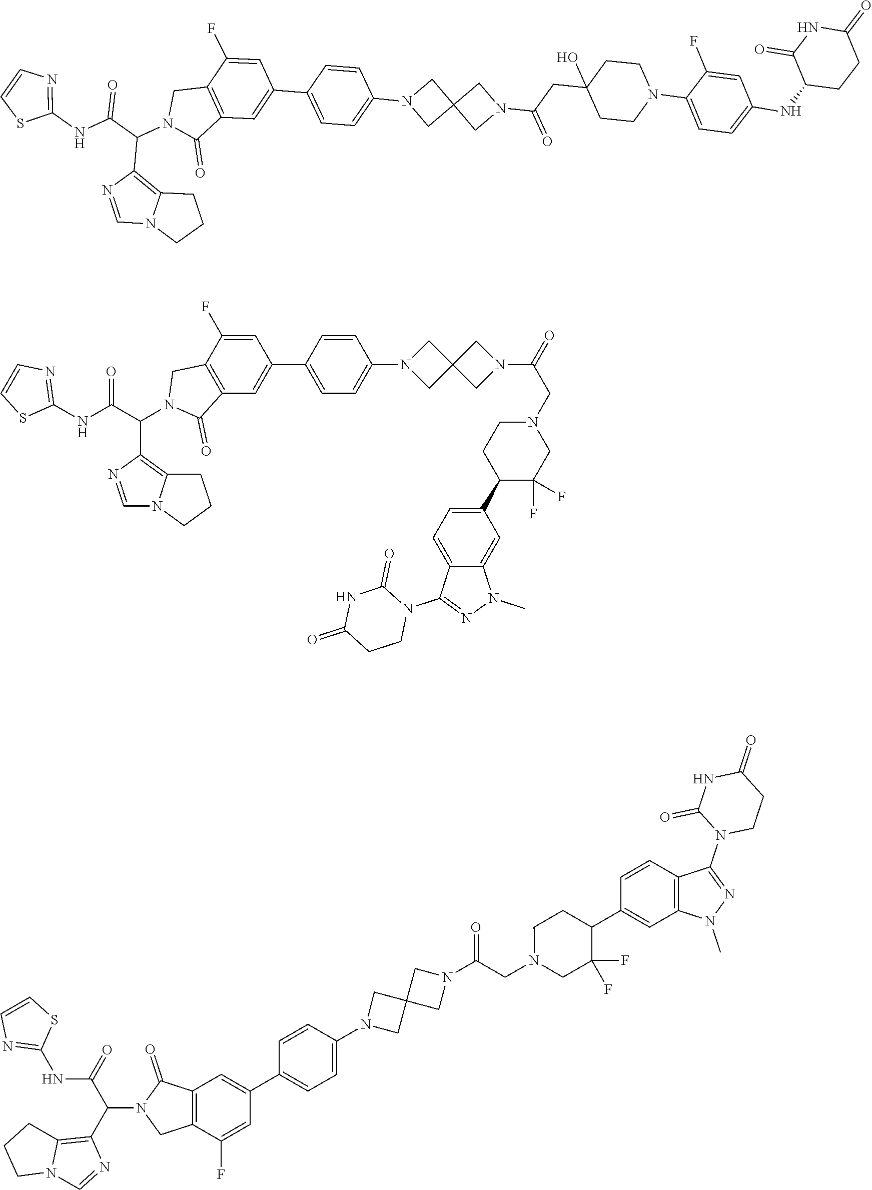 [SMILES: O=C1CC[C@H](Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1 ; Cn1nc(N2CCC(=O)NC2=O)c2ccc([C@H]3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21 ; Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21]

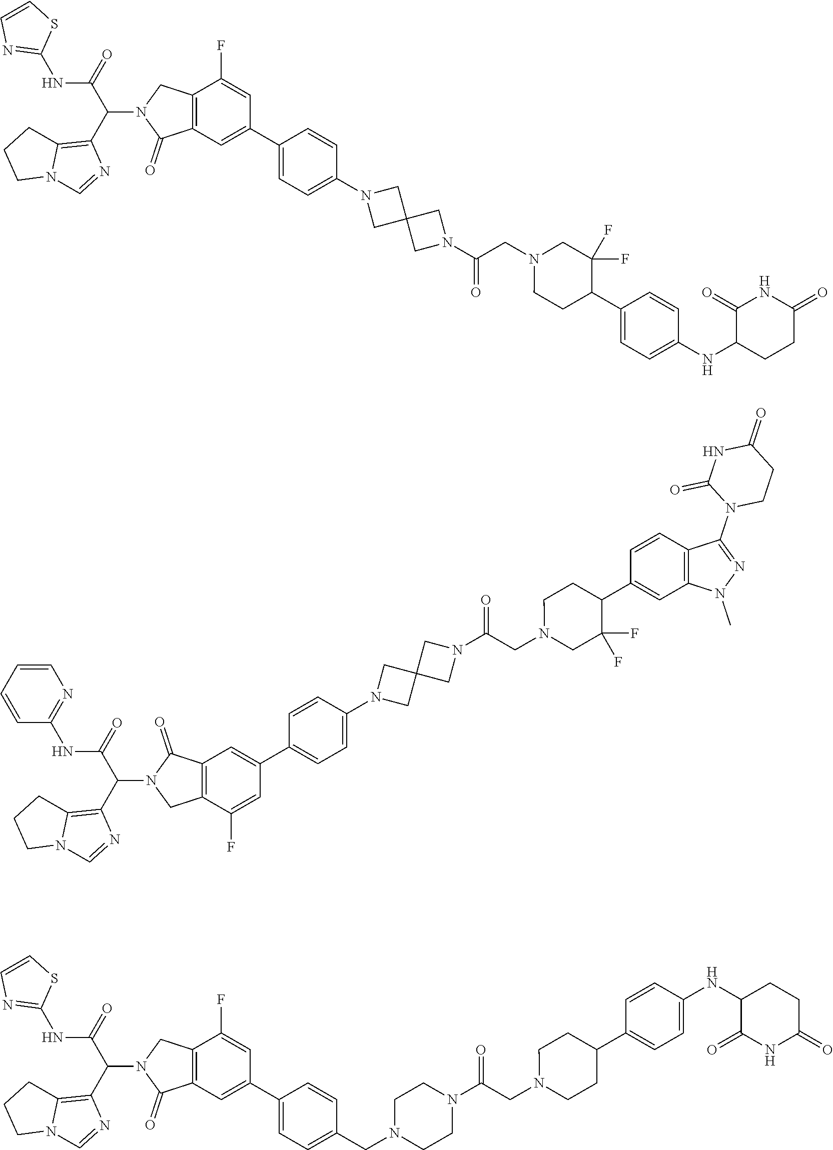 [SMILES: O=C1CCC(Nc2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc2)C(=O)N1 ; Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6ccccn6)c6ncn8c6CCC8)C7)cc4)C5)CC3(F)F)cc21 ; O=C1CCC(Nc2ccc(C3CCN(CC(=O)N4CCN(Cc5ccc(-c6cc(F)c7c(c6)C(=O)N(C(C(=O)Nc6nccs6)c6ncn8c6CCC8)C7)cc5)CC4)CC3)cc2)C(=O)N1]

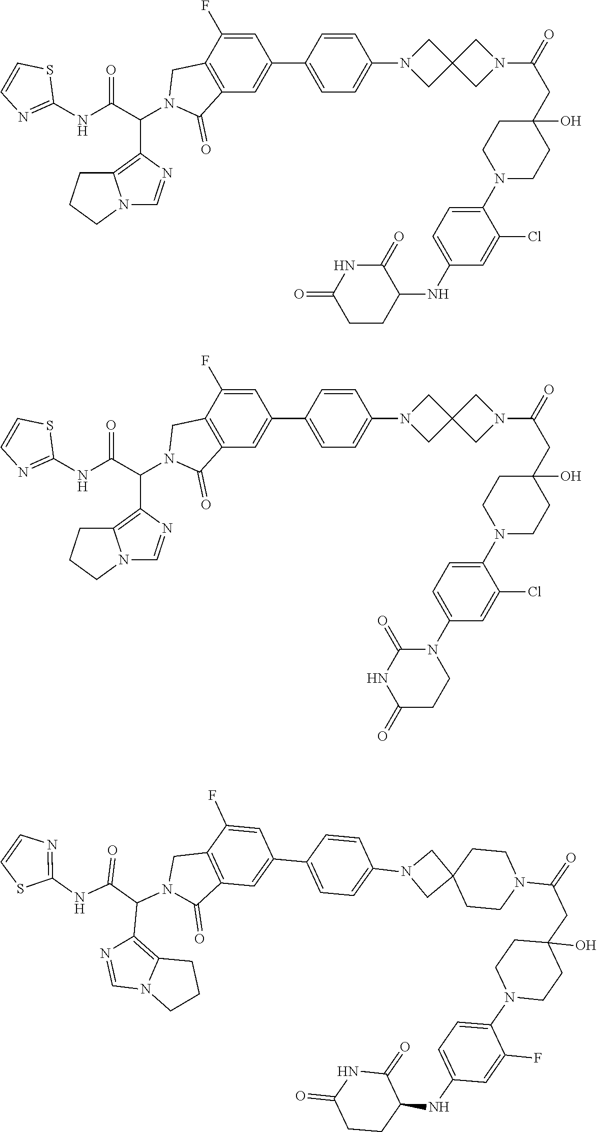 [SMILES: O=C1CCC(Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1 ; O=C1CCN(c2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1 ; O=C1CC[C@H](Nc2ccc(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1]

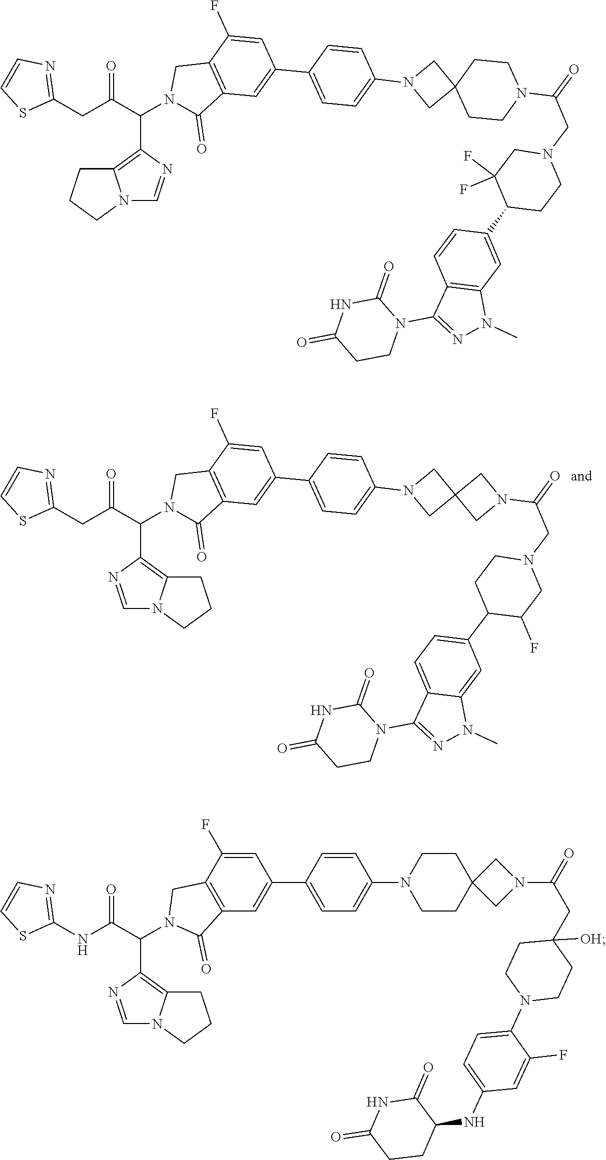 [SMILES: Cn1nc(N2CCC(=O)NC2=O)c2ccc([C@H]3CCN(CC(=O)N4CCC5(CC4)CN(c4ccc(-c6cc(F)c7c(c6)C(=O)N(C(C(=O)Cc6nccs6)c6ncn8c6CCC8)C7)cc4)C5)CC3(F)F)cc21 ; Cn1nc(N2CCC(=O)NC2=O)c2ccc(C3CCN(CC(=O)N4CC5(C4)CN(c4ccc(-c6cc(F)c7c(c6)C(=O)N(C(C(=O)Cc6nccs6)c6ncn8c6CCC8)C7)cc4)C5)CC3F)cc21 ; "and" ; O=C1CC[C@H](Nc2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1]

or a pharmaceutically acceptable salt thereof, to a patient in need thereof is provided.
   5. In certain embodiments a method of treating an EGFR mediated cancer that has metastasized to the brain or CNS comprising administering an effective amount of a Compound of structure:

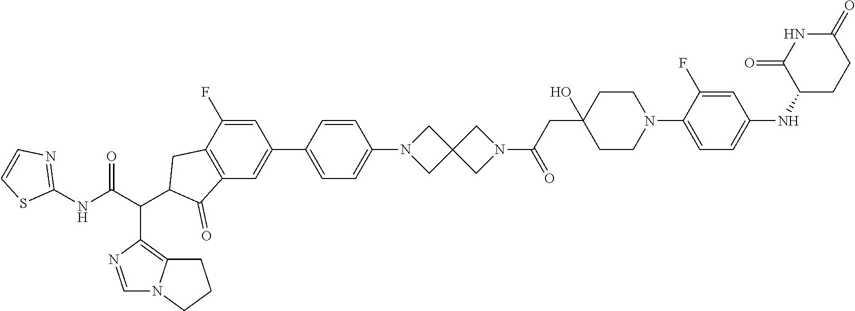 [SMILES: O=C1CC[C@H](Nc2ccc(N3CCC(O)(CC(=O)N4CC5(C4)CN(c4ccc(-c6cc(F)c7c(c6)C(=O)C(C(C(=O)Nc6nccs6)c6ncn8c6CCC8)C7)cc4)C5)CC3)c(F)c2)C(=O)N1]

or a pharmaceutically acceptable salt thereof, to a patient in need thereof is provided.
   6. In certain embodiments a method of treating an EGFR mediated cancer that has metastasized to the brain or CNS comprising administering an effective amount of a Compound of structure:

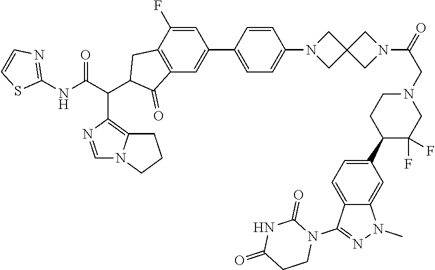 [SMILES: Cn1nc(N2CCC(=O)NC2=O)c2ccc([C@H]3CCN(CC(=O)N4CC5(C4)CN(c4ccc(-c6cc(F)c7c(c6)C(=O)C(C(C(=O)Nc6nccs6)c6ncn8c6CCC8)C7)cc4)C5)CC3(F)F)cc21]

or a pharmaceutically acceptable salt thereof, to a patient in need thereof is provided.
   7. In certain embodiments a method of treating an EGFR mediated cancer that has metastasized to the brain or CNS comprising administering an effective amount of a Compound of structure:

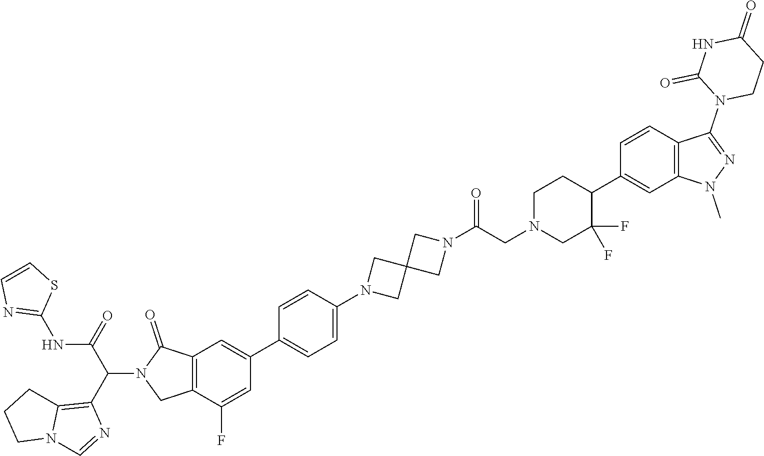 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21]

or a pharmaceutically acceptable salt thereof, to a patient in need thereof is provided.
   8. In certain embodiments a method of treating an EGFR mediated cancer that has metastasized to the brain or CNS comprising administering an effective amount of a Compound of structure:

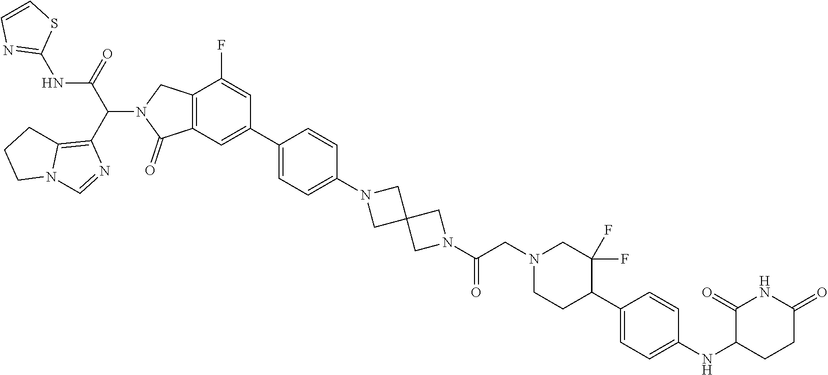 [SMILES: O=C1CCC(Nc2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc2)C(=O)N1]

or a pharmaceutically acceptable salt thereof, to a patient in need thereof is provided.
   9. In certain embodiments a method of treating an EGFR mediated cancer that has metastasized to the brain or CNS comprising administering an effective amount of a Compound of structure:

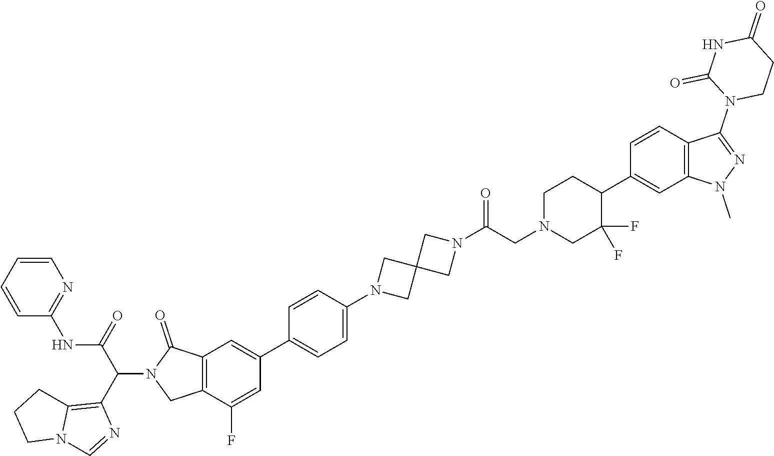 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6ccccn6)c6ncn8c6CCC8)C7)cc4)C5)CC3(F)F)cc21]

or a pharmaceutically acceptable salt thereof, to a patient in need thereof is provided.
   10. In certain embodiments a method of treating an EGFR mediated cancer that has metastasized to the brain or CNS comprising administering an effective amount of a Compound of structure:

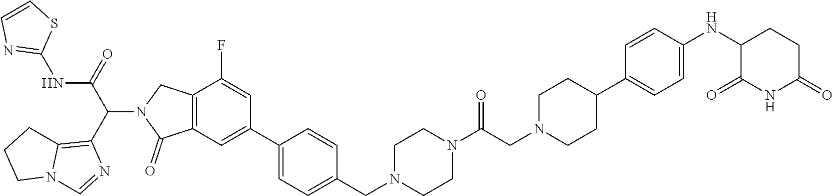 [SMILES: O=C1CCC(Nc2ccc(C3CCN(CC(=O)N4CCN(Cc5ccc(-c6cc(F)c7c(c6)C(=O)N(C(C(=O)Nc6nccs6)c6ncn8c6CCC8)C7)cc5)CC4)CC3)cc2)C(=O)N1]

or a pharmaceutically acceptable salt thereof, to a patient in need thereof is provided.
   11. In certain embodiments a method of treating an EGFR mediated cancer that has metastasized to the brain or CNS comprising administering an effective amount of a Compound of structure:

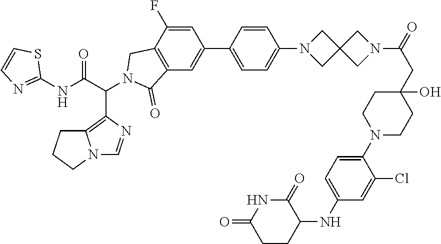 [SMILES: O=C1CCC(Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1]

or a pharmaceutically acceptable salt thereof, to a patient in need thereof is provided.
   12. In certain embodiments a method of treating an EGFR mediated cancer that has metastasized to the brain or CNS comprising administering an effective amount of a Compound of structure:

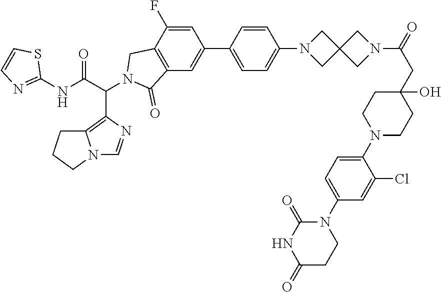 [SMILES: O=C1CCN(c2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1]

or a pharmaceutically acceptable salt thereof, to a patient in need thereof is provided.
   13. In certain embodiments a method of treating an EGFR mediated cancer that has metastasized to the brain or CNS comprising administering an effective amount of a Compound of structure:

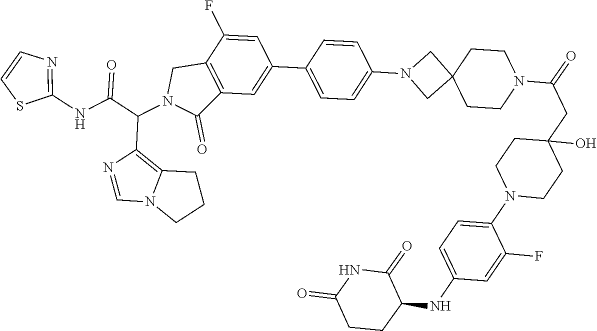 [SMILES: O=C1CC[C@H](Nc2ccc(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1]

or a pharmaceutically acceptable salt thereof, to a patient in need thereof is provided.
   14. In certain embodiments a method of treating an EGFR mediated cancer that has metastasized to the brain or CNS comprising administering an effective amount of a Compound of structure:

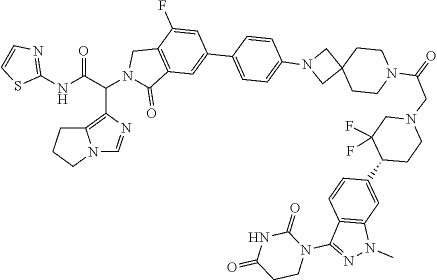 [SMILES: Cn1nc(N2CCC(=O)NC2=O)c2ccc([C@H]3CCN(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3(F)F)cc21]

or a pharmaceutically acceptable salt thereof, to a patient in need thereof is provided.
   15. In certain embodiments a method of treating an EGFR mediated cancer that has metastasized to the brain or CNS comprising administering an effective amount of a Compound of structure:

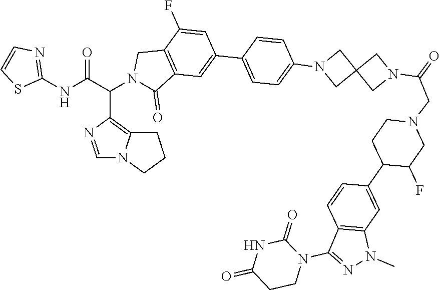 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3F)cc21]

or a pharmaceutically acceptable salt thereof, to a patient in need thereof is provided.
   16. In certain embodiments a method of treating an EGFR mediated cancer that has metastasized to the brain or CNS comprising administering an effective amount of a Compound of structure:

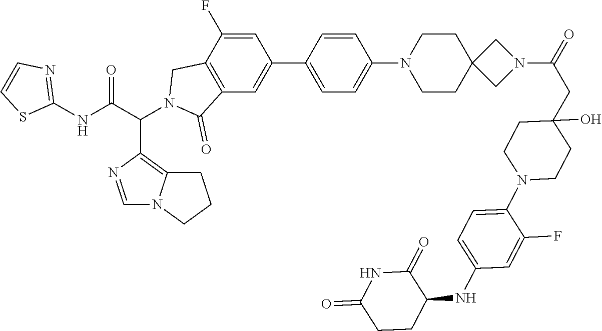 [SMILES: O=C1CC[C@H](Nc2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1]

or a pharmaceutically acceptable salt thereof, to a patient in need thereof is provided.
   17. The method of any one of embodiments 1-16, wherein the patient is a human.
   18. The method of any one of embodiments 1-17, wherein the EGFR mediated cancer is mediated by a mutant EGFR.
   19. The method of embodiment 18, wherein the mutant EGFR has an Exon 21 mutation.
   20. The method of embodiment 19, wherein the mutant EGFR has a L858R mutation.
   21. The method of embodiment 19, wherein the mutant EGFR has a L861Q mutation.
   22. The method of any one of embodiments 18-21, wherein mutant EGFR has a T790M mutation.
   23. The method of any one of embodiments 18-22, wherein mutant EGFR has a C797S mutation.
   24. The method of embodiment 18, wherein the mutant EGFR has a L858R and T790M mutation.
   25. The method of embodiment 18, wherein the mutant EGFR has a L858R, T790M, and C797S mutation.
   26. The method of any one of embodiments 1-25, wherein the Compound is administered as part of a pharmaceutical composition.
   27. The method of any one of embodiments 1-26, wherein the Compound is administered orally.
   28. The method of any one of embodiments 1-26, wherein the Compound is administered parenterally.
   29. The method of any one of embodiments 1-26, wherein the Compound is administered by intravenously.
   30. The method of any one of embodiments 1-29, wherein an ATP site binding EGFR ligand is also administered to the patient in need thereof.
   31. The method of embodiment 30, wherein the ATP site binding EGFR ligand is osimertinib or a pharmaceutically acceptable salt thereof.
   32. The method of embodiment 30, wherein the ATP site binding EGFR ligand is naquotinib or a pharmaceutically acceptable salt thereof.
   33. The method of embodiment 30, wherein the ATP site binding EGFR ligand is mavelertinib or a pharmaceutically acceptable salt thereof
   34. The method of embodiment 30, wherein the ATP site binding EGFR ligand is spebrutinib or a pharmaceutically acceptable salt thereof
   35. The method of any one of embodiments 1-34, wherein the EGFR mediated cancer is lung cancer that has metastasized to the brain or CNS.
   36. The method of any one of embodiments 1-34, wherein the EGFR mediated cancer is non-small cell lung cancer that has metastasized to the brain or CNS.
   37. The method of any one of embodiments 1-34, wherein the EGFR mediated cancer is small cell lung cancer that has metastasized to the brain or CNS.
   38. The method of any one of embodiments 1-34, wherein the EGFR mediated cancer is adenocarcinoma that has metastasized to the brain or CNS.
   39. The method of any one of embodiments 1-34, wherein the EGFR mediated cancer is squamous cell lung cancer that has metastasized to the brain or CNS.
   40. The method of any one of embodiments 1-34, wherein the EGFR mediated cancer is large-cell undifferentiated carcinoma that has metastasized to the brain or CNS.
   41. The method of any one of embodiments 1-34, wherein the EGFR mediated cancer is neuroendocrine carcinoma that has metastasized to the brain or CNS.
   42. The method of any one of embodiments 1-34, wherein the EGFR mediated cancer is sarcomatoid carcinoma, adenosquamous carcinoma, oat-cell cancer, combined small cell carcinoma, lung carcinoid tumor, central carcinoid, peripheral carcinoid, salivary gland-type lung carcinoma, mesothelioma, or a mediastinal tumor that has metastasized to the brain or CNS.
   43. The method of any one of embodiments 1-34, wherein the EGFR mediated cancer is breast cancer that has metastasized to the brain or CNS.
   44. The method of embodiment 43, wherein the EGFR mediated cancer is HER-2 positive breast cancer.
   45. The method of embodiment 43 or 44, wherein the EGFR mediated cancer is ER+ breast cancer.
   46. The method of any one of embodiments 43-45, wherein the EGFR mediated cancer is PR+ breast cancer.
   47. The method of any one of embodiments 1-34, wherein the EGFR mediated cancer is triple negative breast cancer.
   48. The method of any one of embodiments 1-34, wherein the EGFR mediated cancer is colorectal or rectal cancer that has metastasized to the brain or CNS.
   49. The method of any one of embodiments 1-34, wherein the EGFR mediated cancer is head and neck cancer or esophageal cancer that has metastasized to the brain or CNS.
   50. The method of any one of embodiments 1-34, wherein the EGFR mediated cancer is pancreatic cancer that has metastasized to the brain or CNS.
   51. The method of any one of embodiments 1-34, wherein the EGFR mediated cancer is thyroid cancer that has metastasized to the brain or CNS.
   52. The method of any one of embodiments 1-34, wherein the EGFR mediated cancer is ovarian cancer, uterine cancer, or cervical cancer that has metastasized to the brain or CNS.
   53. The method of any one of embodiments 1-34, wherein the EGFR mediated cancer is kidney cancer, liver cancer, or bladder cancer that has metastasized to the brain or CNS.
   54. The method of any one of embodiments 1-34, wherein the EGFR mediated cancer is melanoma that has metastasized to the brain or CNS.
   55. The method of any one of embodiments 1-54, wherein the EGFR mediated cancer has metastasized to the brain.
   56. The method of any one of embodiments 1-54, wherein the EGFR mediated cancer has metastasized to the CNS.
   57. The method of any one of embodiments 1-56, wherein the Compound is administered to a patient with treatment naïve EGFR mediated cancer.
   58. The method of any one of embodiments 1-56, wherein the EGFR mediated cancer is relapsed.
   59. The method of any one of embodiments 1-56, wherein the EGFR mediated cancer is refractory.
   60. The method of any one of embodiments 1-56, wherein the EGFR mediated cancer is relapsed and refractory.
   61. In certain embodiments the use of a Compound described herein (for example a Compound used in any one of embodiments 1-60) for the manufacture of a medicament to treat a disorder described herein (for example a disorder of any one of embodiments 1-60) is provided.
   62. In certain embodiments the use of a Compound described herein (for example a Compound used in any one of embodiments 1-60) in the treatment of a disorder described herein (for example a disorder of any one of embodiments 1-60) is provided.
   63. In certain embodiments the Compound described herein (for example a Compound used in any one of embodiments 1-60) for use in the treatment of a disorder described herein (for example a disorder of any one of embodiments 1-60) is provided.

### Additional Embodiments of the Present Invention

### Chirality Embodiments

The compounds described herein may have multiple stereocenters (e.g., chiral carbon atoms) including for example one or more stereocenters in the E3 ligase binding moiety (for example

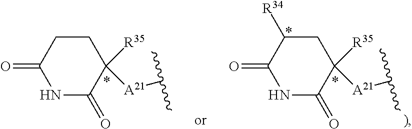

one or more stereocenters in the linker, and/or at least one stereocenter in the EGFR binding ligand moiety of the molecule

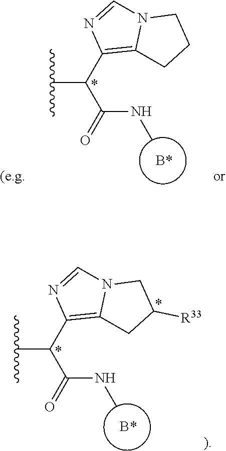

In certain embodiments, the EGFR-degrading compound described herein is provided without regard to stereochemistry. In other embodiments, the EGFR-degrading compound may have one or more chiral carbons presented in an enantiomerically enriched (i.e., greater than about 50%, 60%, 70%, 80% or 90% pure) or even substantially pure form (greater than about 95%, 98% or 99% pure) of R and S stereochemistry. In certain aspects, the EGFR-degrading compound has two enantiomerically enriched and/or substantially pure stereocenters. In one sub-aspect of this, the two enantiomerically enriched and/or substantially pure stereocenters are located in the ligase-binding moiety of the compound and the linker; or alternatively there are two in the linker. In another sub-aspect, there are three enantiomerically enriched and/or substantially pure stereocenters, with one in the ligase-binding moiety of the compound and two in the linker. In yet another sub-aspect of this, there are three enantiomerically enriched and/or substantially pure stereocenters, with one in the ligase-binding moiety of the compound and two in the linker. In another aspect, in any of these embodiments, aspects or sub-aspects, in addition, the EGFR binding ligand moiety is enantiomerically enriched or in substantially pure form.

It has been observed that in some embodiments, the chiral carbon in the EGFR binding ligand moiety adjacent to the amide may easily racemize between stereoisomers under the conditions of use, and therefore in certain embodiments, is not considered for purposes of stereochemistry designation.

In certain embodiments one stereocenter is in the R configuration and any others present are either enantiomerically enriched or substantially pure. In certain embodiments one stereocenter is in the S configuration and any others present are either enantiomerically enriched or substantially pure.

In certain embodiments one stereocenter is in the R configuration and any others present are without regard to stereochemistry, enantiomerically enriched or substantially pure. In certain embodiments one stereocenter is in the S configuration and any others present are without regard to stereochemistry, enantiomerically enriched or substantially pure.

In certain embodiments there is one stereocenter in the E3 ligase binding moiety (disregarding the stereocenter in the EGFR binding ligand moiety) and it is enantiomerically enriched or substantially pure in the R-configuration, as indicated below. In another embodiment there is one stereocenter in the E3 ligase binding moiety (disregarding the stereocenter in the EGFR binding ligand moiety) and it is enantiomerically enriched or substantially pure in the 5-configuration, as indicated below.

In certain embodiments

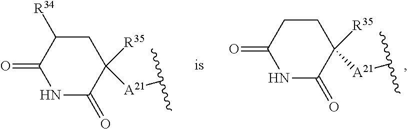

wherein R³⁴ is hydrogen.

In certain embodiments

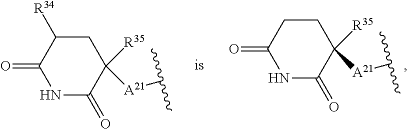

wherein R³⁴ is hydrogen.

In certain embodiments

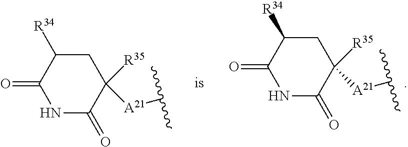

In certain embodiments

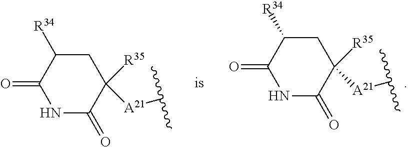

In certain embodiments

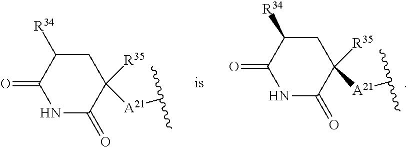

In certain embodiments

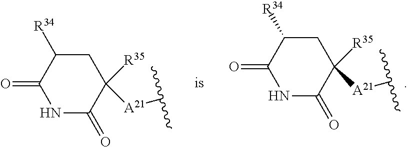

In certain embodiments there is one stereocenter in the linker portion and it is a mixture of R- and S-configuration. In another embodiment there is one stereocenter in the linker portion and it is enantiomerically enriched or substantially pure R-configuration. In another embodiment there is one stereocenter in the linker portion and it is enantiomerically enriched or substantially pure S-configuration.

In certain embodiments the linker contains one or more moieties with a chiral center. Non-limiting examples include heterocycle with an enantiomerically enriched or substantially pure stereocenter for example piperidine with a substituent meta- or ortho to the nitrogen or linking in the meta- or ortho-configuration; piperazine with a substituent or linking in the meta- or ortho-configuration; pyrrolidinone with or without a substituent; and pyrrolidine with or without a substituent.

Additional non-limiting examples of linker moieties with at least one chiral center include an alkyl with an enantiomerically enriched or substantially pure stereocenter; an alkene with an enantiomerically enriched or substantially pure stereocenter; an alkyne with an enantiomerically enriched or substantially pure stereocenter; a haloalkyl with an enantiomerically enriched or substantially pure stereocenter; an alkoxy with an enantiomerically enriched or substantially pure stereocenter; an aliphatic group with an enantiomerically enriched or substantially pure stereocenter; a heteroaliphatic group with an enantiomerically enriched or substantially pure stereocenter; and a cycloalkyl with an enantiomerically enriched or substantially pure stereocenter

In certain embodiments the linker includes

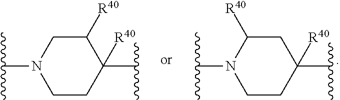

In certain embodiments the linker includes

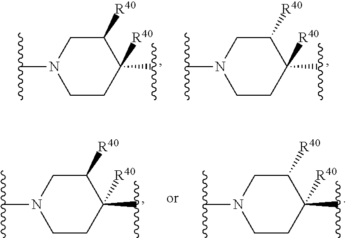

In certain embodiments the linker includes

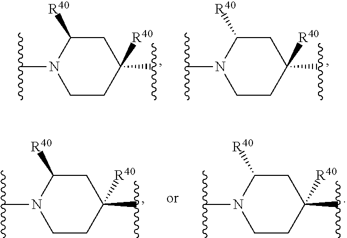

In certain embodiments the linker includes

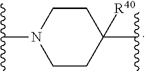

In certain embodiments the linker includes

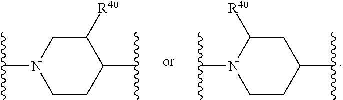

In certain embodiments the linker includes

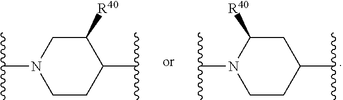

In certain embodiments the linker includes

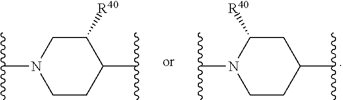

In certain embodiments the linker includes

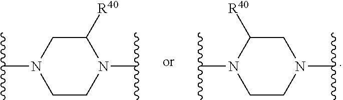

In certain embodiments the linker includes

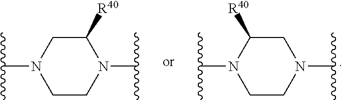

In certain embodiments the linker includes

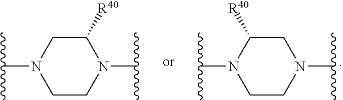

In certain embodiments the linker includes

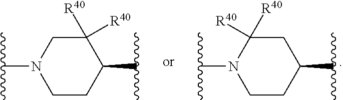

In certain embodiments the linker includes

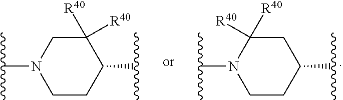

In certain embodiments the linker includes

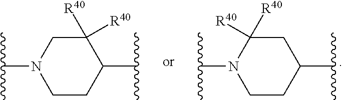

In certain embodiments, there is at least one stereocenter in the EGFR ligand portion which is a mixture of R and S. In another embodiment there is at least one stereocenter in the EGFR ligand portion and it is enantiomerically enriched or substantially pure in the R-configuration. In another embodiment there is at least one stereocenter in the EGFR ligand portion and it is enantiomerically enriched or substantially pure in the S-configuration.

In certain embodiments

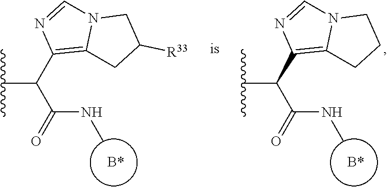

wherein R³³ is hydrogen.

In certain embodiments

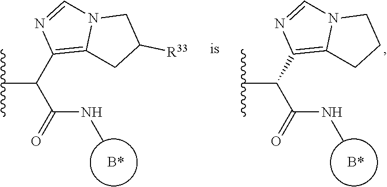

wherein R³³ is hydrogen.

In certain embodiments

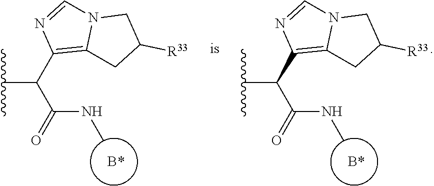

In certain embodiments

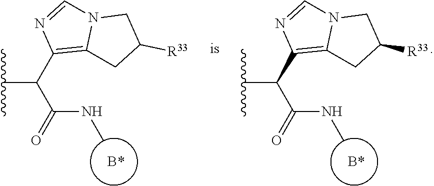

In certain embodiments

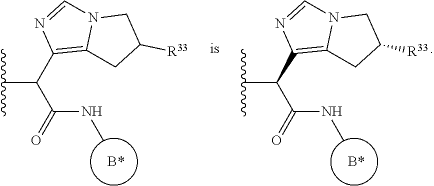

In certain embodiments

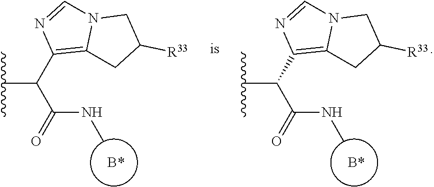

In certain embodiments

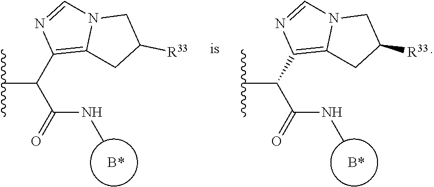

In certain embodiments

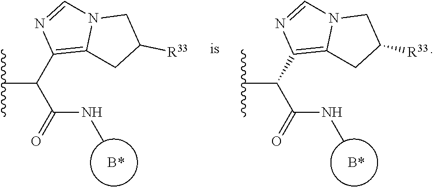

### Embodiments of Alkyl

In certain embodiments “alkyl” is a C₁-C₁₀alkyl, C₁-C₉alkyl, C₁-C₈alkyl, C₁-C₇alkyl, C₁-C₆alkyl, C₁-C₈alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment “alkyl” is “optionally substituted” with 1, 2, 3, or 4 R³¹ substituents.

### Embodiments of Cycloalkyl

In certain embodiments “cycloalkyl” is a C₃-C₈cycloalkyl, C₃-C₇cycloalkyl, C₃-C₆cycloalkyl, C₃-C₅cycloalkyl, C₃-C₄cycloalkyl, C₄-C₈cycloalkyl, C₅-C₈cycloalkyl, or C₆-C₈cycloalkyl.

In certain embodiments “cycloalkyl” has three carbons.

In certain embodiments “cycloalkyl” has four carbons.

In certain embodiments “cycloalkyl” has five carbons.

In certain embodiments “cycloalkyl” has six carbons.

In certain embodiments “cycloalkyl” has seven carbons.

In certain embodiments “cycloalkyl” has eight carbons.

In certain embodiments “cycloalkyl” has nine carbons.

In certain embodiments “cycloalkyl” has ten carbons.

Non-limiting examples of “cycloalkyl” include: cyclopropyl, cyclobutyl, cyclopentyl, cyclohexyl, cycloheptyl, cyclooctyl, and cyclodecyl.

In an alternative embodiment “cycloalkyl” is “optionally substituted” with 1, 2, 3, or 4 R³¹ substituents.

### Embodiments of Haloalkyl

In certain embodiments “haloalkyl” is a C₁-C₁₀haloalkyl, C₁-C₉haloalkyl, C₁-C₈haloalkyl, C₁-C₇haloalkyl, C₁-C₆haloalkyl, C₁-C₅haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has three carbons.

In certain embodiments “haloalkyl” has four carbons.

In certain embodiments “haloalkyl” has five carbons.

In certain embodiments “haloalkyl” has six carbons.

Non-limiting examples of “haloalkyl” include:

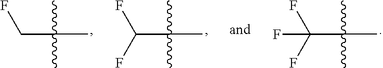

Additional non-limiting examples of “haloalkyl” include:

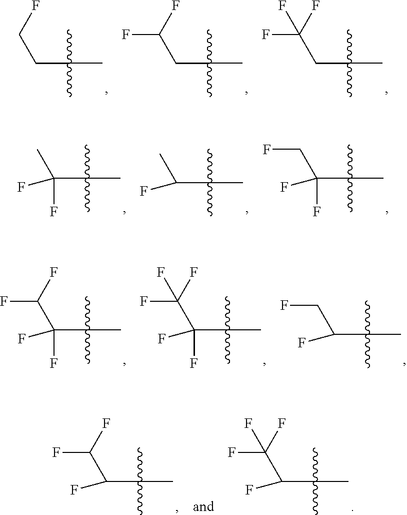

Additional non-limiting examples of “haloalkyl” include:

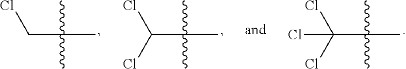

Additional non-limiting examples of “haloalkyl” include:

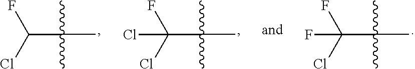

### Embodiments of Heterocycle

In certain embodiments “heterocycle” refers to a cyclic ring with one nitrogen and 3, 4, 5, 6, 7, or 8 carbon atoms.

In certain embodiments “heterocycle” refers to a cyclic ring with one nitrogen and one oxygen and 3, 4, 5, 6, 7, or 8 carbon atoms.

In certain embodiments “heterocycle” refers to a cyclic ring with two nitrogens and 3, 4, 5, 6, 7, or 8 carbon atoms.

In certain embodiments “heterocycle” refers to a cyclic ring with one oxygen and 3, 4, 5, 6, 7, or 8 carbon atoms.

In certain embodiments “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of“heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocycle ring.

Non-limiting examples of “heterocycle” also include:

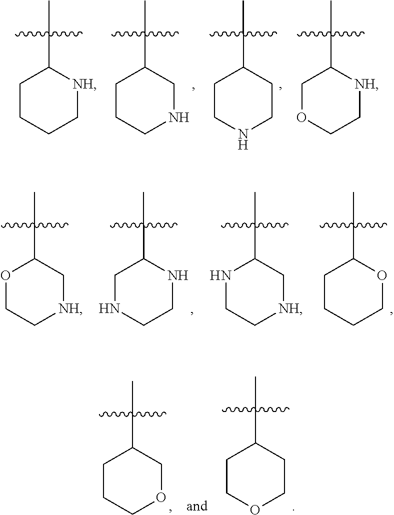

Additional non-limiting examples of “heterocycle” include:

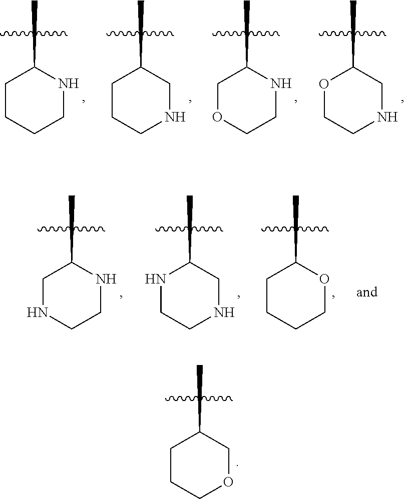

Additional non-limiting examples of “heterocycle” include:

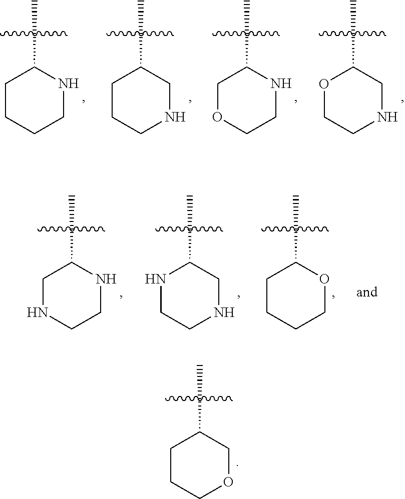

Non-limiting examples of “heterocycle” also include:

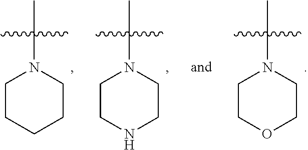

Non-limiting examples of “heterocycle” also include:

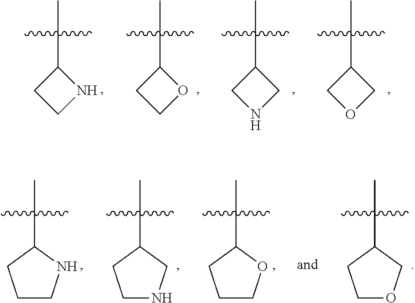

Additional non-limiting examples of “heterocycle” include:

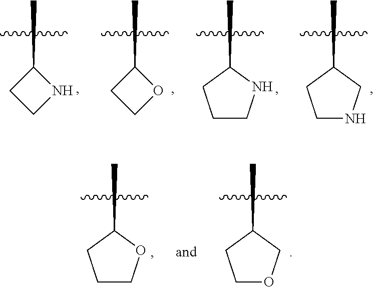

Additional non-limiting examples of “heterocycle” include:

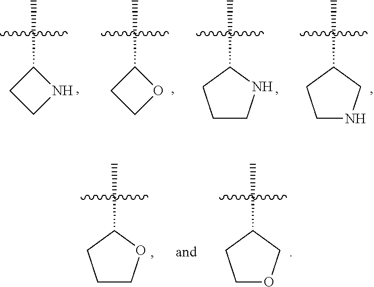

In an alternative embodiment “heterocycle” is “optionally substituted” with 1, 2, 3, or 4 R³¹ substituents.

### Embodiments of Heteroaryl

In certain embodiments “heteroaryl” is a 5 membered aromatic group containing 1, 2, 3, or 4 nitrogen atoms.

Non-limiting examples of 5 membered “heteroaryl” groups include pyrrole, furan, thiophene, pyrazole, imidazole, triazole, tetrazole, isoxazole, oxazole, oxadiazole, oxatriazole, isothiazole, thiazole, thiadiazole, and thiatriazole.

Additional non-limiting examples of 5 membered “heteroaryl” groups include:

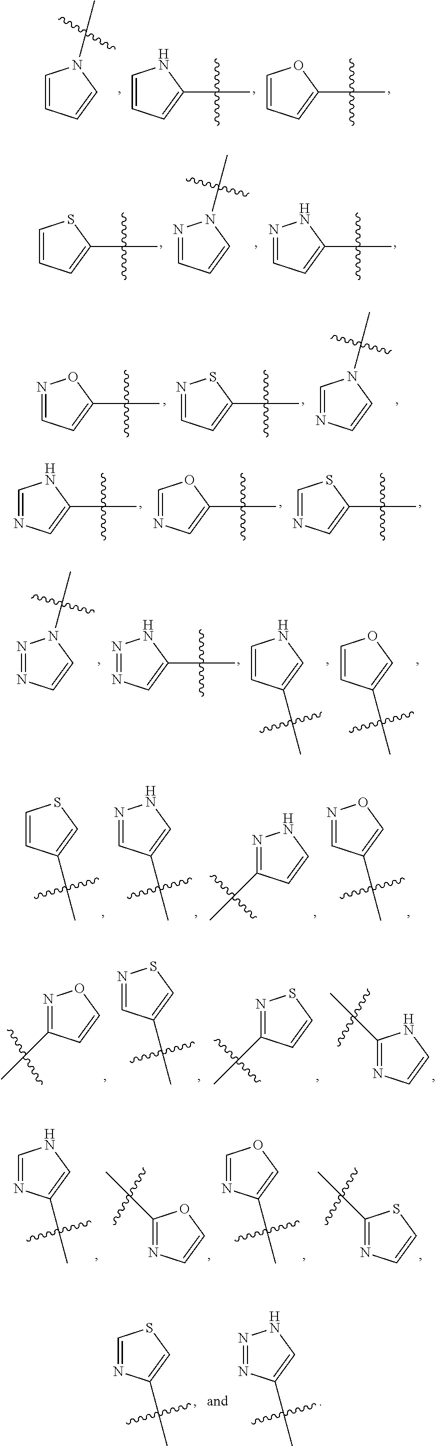

In certain embodiments “heteroaryl” is a 6 membered aromatic group containing 1, 2, or 3 nitrogen atoms (i.e., pyridinyl, pyridazinyl, triazinyl, pyrimidinyl, and pyrazinyl).

Non-limiting examples of 6 membered “heteroaryl” groups with 1 or 2 nitrogen atoms include:

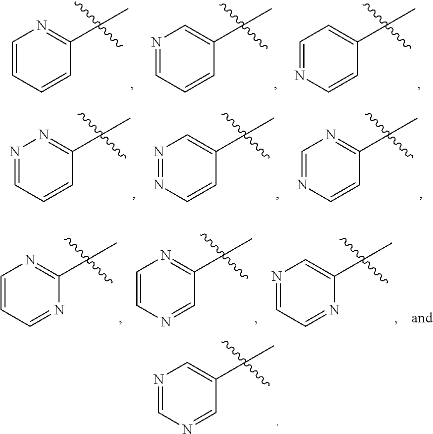

In certain embodiments “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

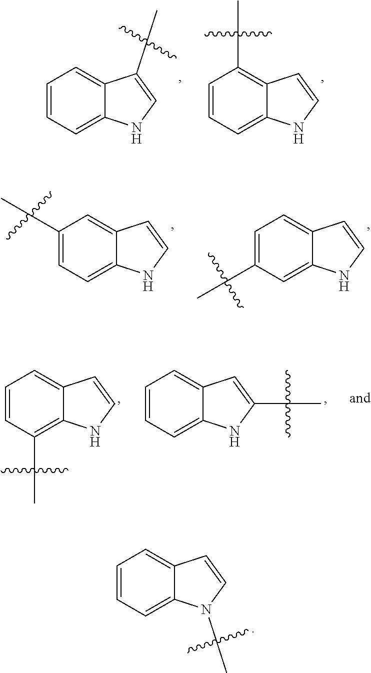

Additional non-limiting examples of “heteroaryl” groups that are bicyclic include:

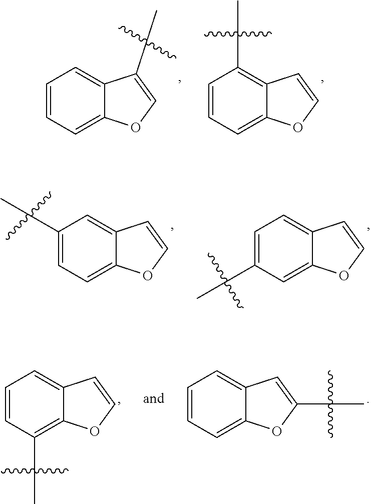 [SMILES: *c1coc2ccccc12 ; *c1cccc2occc12 ; *c1ccc2occc2c1 ; *c1ccc2ccoc2c1 ; *c1cccc2ccoc12 ; *c1cc2ccccc2o1]

Additional non-limiting examples of “heteroaryl” groups that are bicyclic include:

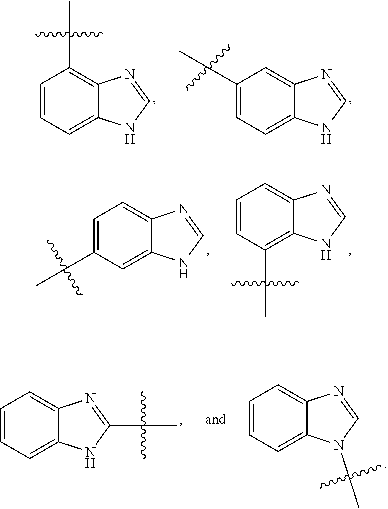

In certain embodiments “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

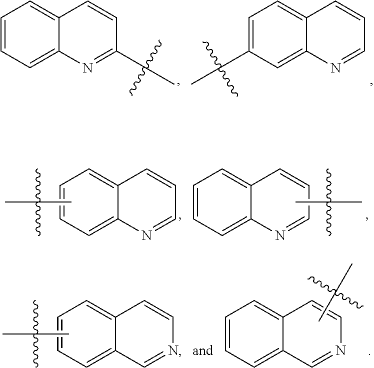

In an alternative embodiment “heteroaryl” is “optionally substituted” with 1, 2, 3, or 4 R³¹ substituents.

### Embodiments of Aryl

In certain embodiments aryl is phenyl.

In certain embodiments aryl is napthyl.

In an alternative embodiment “aryl” is “optionally substituted” with 1, 2, 3, or 4 R³¹ substituents.

### Embodiments of Bicycle

The term “bicycle” refers to a ring system wherein two rings share at least one atom in common. These rings can be spirocyclic or fused together and each ring is independently selected from carbocycle, heterocycle, aryl, and heteroaryl. Non-limiting examples of bicycle groups include:

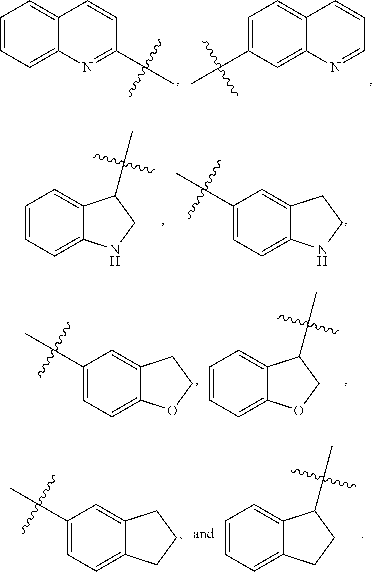

When the term “bicycle” is used in the context of a bivalent residue such as Linker the attachment points can be on separate rings or on the same ring. In certain embodiments both attachment points are on the same ring. In certain embodiments both attachment points are on different rings. Non-limiting examples of bivalent bicycle groups include:

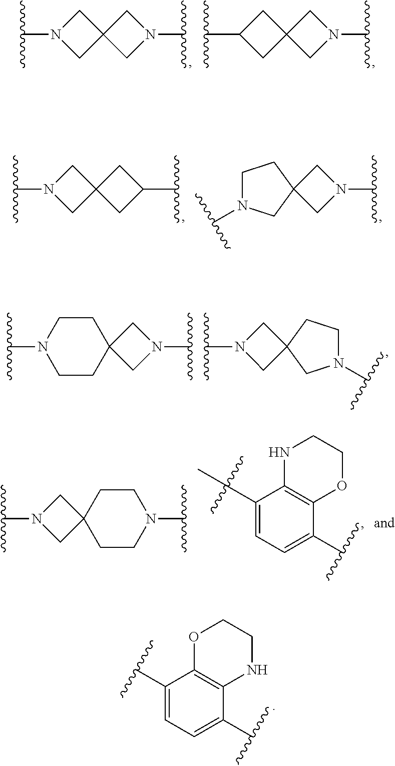

Additional non-limiting examples of bivalent bicycle include:

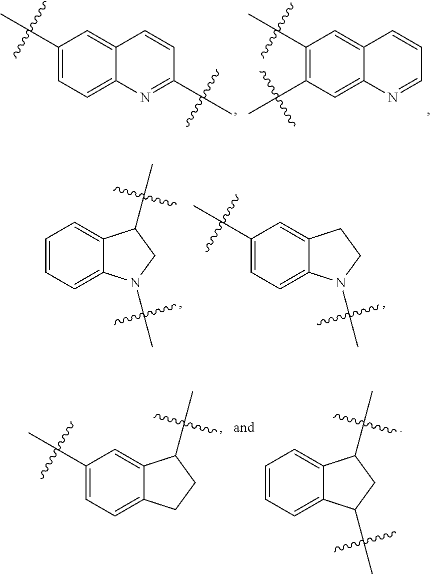 [SMILES: *c1ccc2nc(*)ccc2c1 ; *c1cc2cccnc2cc1* ; *C1CN(*)c2ccccc21 ; *c1ccc2c(c1)CCN2* ; *c1ccc2c(c1)C(*)CC2 ; *C1CC(*)c2ccccc21]

In an alternative embodiment “bicycle” is “optionally substituted” with 1, 2, 3, or 4 R³¹ substituents.

### Embodiments of Optional Substituents

In certain embodiments wherein a variable can be optionally substituted it is not substituted.

In certain embodiments wherein a variable can be optionally substituted it is substituted with 1 substituent.

In certain embodiments wherein a variable can be optionally substituted it is substituted with 2 substituents.

In certain embodiments wherein a variable can be optionally substituted it is substituted with 3 substituents.

In certain embodiments wherein a variable can be optionally substituted it is substituted with 4 substituents.

In one alternative embodiment any suitable group may be present on a “substituted” or “optionally substituted” position if indicated that forms a stable molecule and meets the desired purpose of the invention and includes, but is not limited to, e.g., halogen (which can independently be F, Cl, Br or I); cyano; hydroxyl; nitro; azido; alkanoyl (such as a C₂-C₆ alkanoyl group); carboxamide; alkyl, cycloalkyl, alkenyl, alkynyl, alkoxy, aryloxy such as phenoxy; thioalkyl including those having one or more thioether linkages; alkylsulfinyl; alkylsulfonyl groups including those having one or more sulfonyl linkages; aminoalkyl groups including groups having more than one N atoms; aryl (e.g., phenyl, biphenyl, naphthyl, or the like, each ring either substituted or unsubstituted); arylalkyl having for example, 1 to 3 separate or fused rings and from 6 to about 14 or 18 ring carbon atoms, with benzyl being an exemplary arylalkyl group; arylalkoxy, for example, having 1 to 3 separate or fused rings with benzyloxy being an exemplary arylalkoxy group; or a saturated or partially unsaturated heterocycle having 1 to 3 separate or fused rings with one or more N, O or S atoms, or a heteroaryl having 1 to 3 separate or fused rings with one or more N, O or S atoms, e.g. coumarinyl, quinolinyl, isoquinolinyl, quinazolinyl, pyridyl, pyrazinyl, pyrimidinyl, furanyl, pyrrolyl, thienyl, thiazolyl, triazinyl, oxazolyl, isoxazolyl, imidazolyl, indolyl, benzofuranyl, benzothiazolyl, tetrahydrofuranyl, tetrahydropyranyl, piperidinyl, morpholinyl, piperazinyl, and pyrrolidinyl. Such groups may be further substituted, e.g. with hydroxy, alkyl, alkoxy, halogen and amino.

### Embodiments of Aliphatic and Heteroaliphatic

In certain embodiments “aliphatic” refers to a saturated or unsaturated, straight, branched, or cyclic hydrocarbon. In these embodiments aliphatic is intended to include, but is not limited to, alkyl, alkenyl, alkynyl, cycloalkyl, cycloalkenyl, and cycloalkynyl moieties, and thus incorporates each of these definitions. In certain embodiments, “aliphatic” is used to indicate those aliphatic groups having 1-20 carbon atoms. The aliphatic chain can be, for example, mono-unsaturated, di-unsaturated, tri-unsaturated, or polyunsaturated, or alkynyl. Unsaturated aliphatic groups can be in a cis or trans configuration. In certain embodiments, the aliphatic group contains from 1 to about 12 carbon atoms, more generally from 1 to about 6 carbon atoms or from 1 to about 4 carbon atoms. In certain embodiments, the aliphatic group contains from 1 to about 8 carbon atoms. In certain embodiments, the aliphatic group is C₁-C₂, C₁-C₃, C₁-C₄, C₁-C₅ or C₁-C₆. The specified ranges as used herein indicate an aliphatic group having each member of the range described as an independent species. For example, the term C₁-C₆ aliphatic as used herein indicates a straight or branched alkyl, alkenyl, or alkynyl group having from 1, 2, 3, 4, 5, or 6 carbon atoms and is intended to mean that each of these is described as an independent species. For example, the term C₁-C₄ aliphatic as used herein indicates a straight or branched alkyl, alkenyl, or alkynyl group having from 1, 2, 3, or 4 carbon atoms and is intended to mean that each of these is described as an independent species. In certain embodiments, the aliphatic group is substituted with one or more functional groups that results in the formation of a stable moiety.

In certain embodiments “heteroaliphatic” refers to an aliphatic moiety that contains at least one heteroatom in the chain, for example, an amine, carbonyl, carboxy, oxo, thio, phosphate, phosphonate, nitrogen, phosphorus, silicon, or boron atoms in place of a carbon atom. In certain embodiments, the only heteroatom is nitrogen. In certain embodiments, the only heteroatom is oxygen. In certain embodiments, the only heteroatom is sulfur. In certain embodiments “heteroaliphatic” is intended herein to include, but is not limited to, heteroalkyl, heteroalkenyl, heteroalkynyl, heterocycloalkyl, heterocycloalkenyl, and heterocycloalkynyl moieties. In certain embodiments, “heteroaliphatic” is used to indicate a heteroaliphatic group (cyclic, acyclic, substituted, unsubstituted, branched or unbranched) having 1-20 carbon atoms. In certain embodiments, the heteroaliphatic group is optionally substituted in a manner that results in the formation of a stable moiety. Nonlimiting examples of heteroaliphatic moieties are polyethylene glycol, polyalkylene glycol, amide, polyamide, polylactide, polyglycolide, thioether, ether, alkyl-heterocycle-alkyl, —O-alkyl-O-alkyl, alkyl-O-haloalkyl, etc.

### Embodiments of A and A*

In certain embodiments A* is

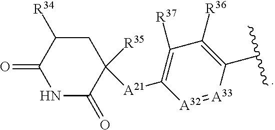

In certain embodiments A* is

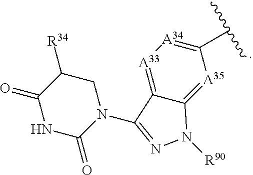

In certain embodiments A* is

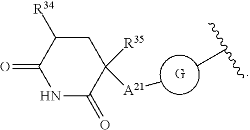

In certain embodiments A* is

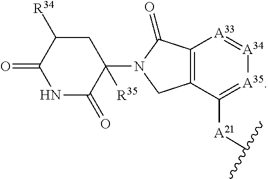

In certain embodiments A* is

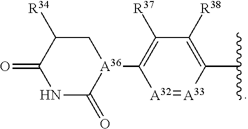

In certain embodiments A* is

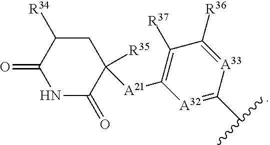

In certain embodiments R³⁴ and R³⁵ combine to form a CH₂.

In certain embodiments R³⁴ is H.

In certain embodiments R³⁵ is H.

In certain embodiments A¹ is NH.

In certain embodiments A¹ is O.

In certain embodiments A²¹ is NH.

In certain embodiments A²¹ is O.

In certain embodiments A²¹ is CH₂.

In certain embodiments A²¹ is NR¹⁰⁰.

In certain embodiments A³², A³³, A³⁴, and A³⁵ are each selected from CH, C-halogen, and CF.

In certain embodiments A³² is CH.

In certain embodiments A³² is CF.

In certain embodiments A³² is CR⁴².

In certain embodiments A³² is N.

In certain embodiments A³³ is CH.

In certain embodiments A³³ is CF.

In certain embodiments A³³ is CR⁴².

In certain embodiments A³³ is N.

In certain embodiments A³⁴ is CH.

In certain embodiments A³⁴ is CF.

In certain embodiments A³⁴ is CR⁴².

In certain embodiments A³⁴ is N.

In certain embodiments A³⁵ is CH.

In certain embodiments A³⁵ is CF.

In certain embodiments A³⁵ is CR⁴².

In certain embodiments A³⁵ is N.

In certain embodiments A³⁶ is N.

In certain embodiments R⁹⁰ is hydrogen.

In certain embodiments R⁹⁰ is C₁-C₃ alkyl.

In certain embodiments R⁹⁰ is C₃₋₆-cycloalkyl.

In certain embodiments R⁹⁰ is methyl.

In certain embodiments A or A* is

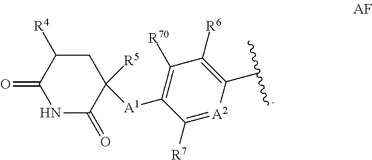

In certain embodiments A or A* is

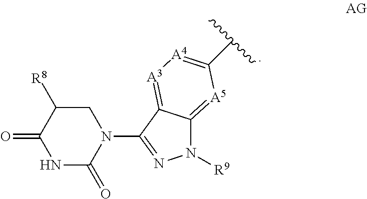

In certain embodiments A or A* is

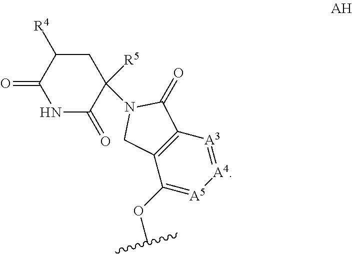

In certain embodiments, A or A* is selected from:

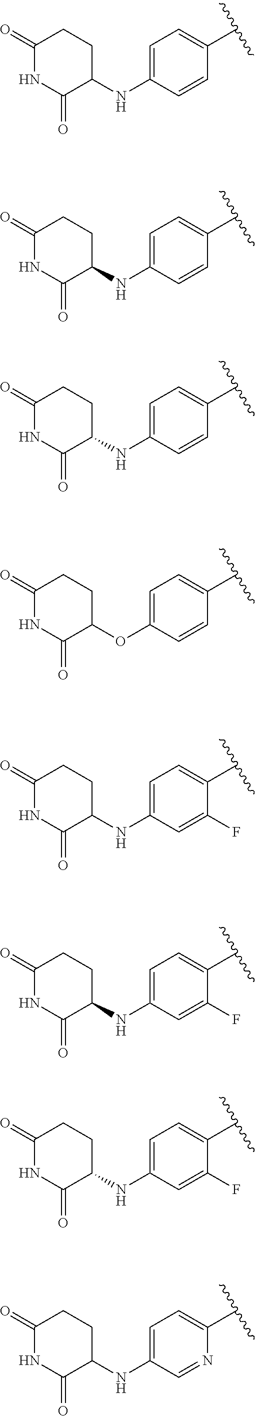

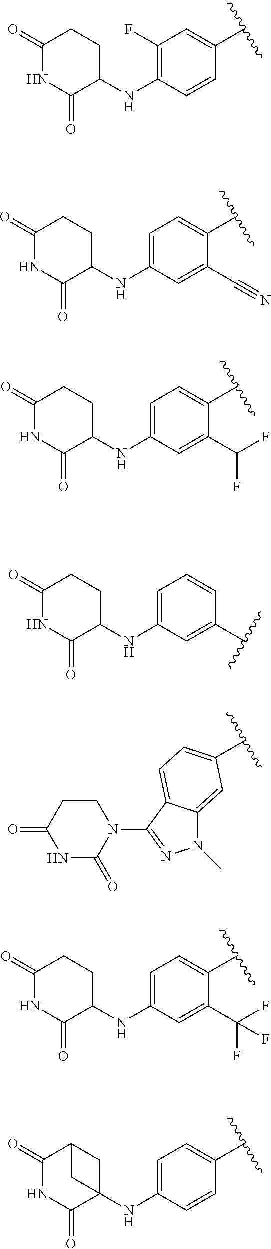

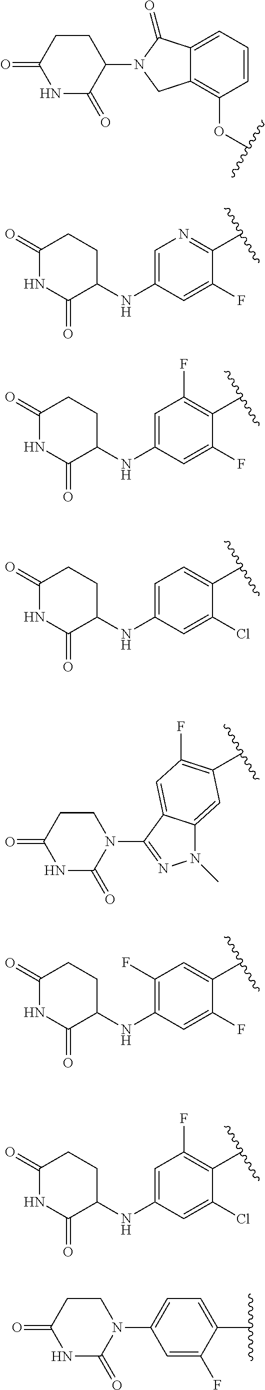

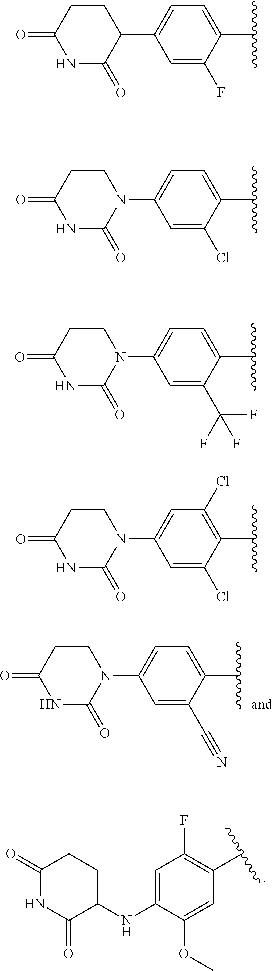

### Embodiments of B and B*

In certain embodiments B or B* is

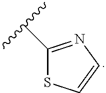 [SMILES: *c1nccs1]

In certain embodiments B or B* is

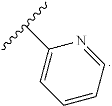 [SMILES: *c1ccccn1]

In certain embodiments B* is heteroaryl.

In certain embodiments B* is heteroaryl substituted with one R³¹ group.

In certain embodiments B* is aryl.

In certain embodiments B* is aryl substituted with one R³¹ group.

In certain embodiments B* is

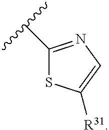

In certain embodiments B* is

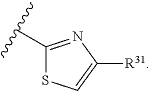

In certain embodiments B* is

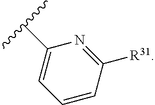

In certain embodiments B* is

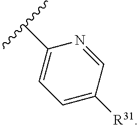

In certain embodiments B* is

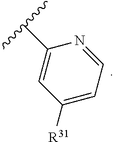

In certain embodiments B* is

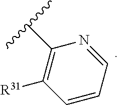

### Embodiments of y

In certain embodiments y is 0.

In certain embodiments y is 1.

In certain embodiments y is 2. In certain embodiments y is 3.

### Embodiments of R³¹

In certain embodiments at least one R³¹ is halogen.

In certain embodiments at least one R³¹ is F.

In certain embodiments at least one R³¹ is Cl.

In certain embodiments at least one R³¹ is C₁₋₆-alkyl.

In certain embodiments at least one R³¹ is halo-C₁₋₆-alkyl.

In certain embodiments one R³¹ is halogen.

In certain embodiments one R³¹ is F.

In certain embodiments one R³¹ is Cl.

In certain embodiments one R³¹ is C₁₋₆-alkyl.

In certain embodiments one R³¹ is cyano.

In certain embodiments one R³¹ is C₁₋₆-alkoxy.

In certain embodiments one R³¹ is halo-C₁₋₆-alkoxy.

In certain embodiments one R³¹ is C₃₋₈-cycloalkyl.

In certain embodiments one R³¹ is halo-C₃₋₈-cycloalkyl.

In certain embodiments R³¹ is selected from halogen, C₁₋₆-alkoxy, and C₁₋₆-alkyl.

In certain embodiments R³¹ is selected from F, Cl, methoxy, and methyl.

### Embodiments of R³⁶ and R³⁷

In certain embodiments R³⁶ and R³⁷ together are combined to form a 5-membered cycle optionally substituted with 1, 2, or 3 R³¹ substituents.

In certain embodiments R³⁶ and R³⁷ together are combined to form a 6-membered cycle optionally substituted with 1, 2, or 3 R³¹ substituents.

In certain embodiments R³⁶ and R³⁷ together are combined to form a 5-membered cycloalkyl optionally substituted with 1, 2, or 3 R³¹ substituents.

In certain embodiments R³⁶ and R³⁷ together are combined to form a 6-membered cycloalkyl optionally substituted with 1, 2, or 3 R³¹ substituents.

In certain embodiments R³⁶ and R³⁷ together are combined to form a 5-membered heteroaryl optionally substituted with 1, 2, or 3 R³¹ substituents.

In certain embodiments R³⁶ and R³⁷ together are combined to form a 6-membered heteroaryl optionally substituted with 1, 2, or 3 R³¹ substituents.

In certain embodiments R³⁶ and R³⁷ together are combined to form a 5-membered heterocycle optionally substituted with 1, 2, or 3 R³¹ substituents.

In certain embodiments R³⁶ and R³⁷ together are combined to form a 6-membered heterocycle optionally substituted with 1, 2, or 3 R³¹ substituents.

In certain embodiments R³⁶ and R³⁷ together are combined to form a morpholine optionally substituted with 1, 2, or 3 R³¹ substituents.

In certain embodiments R³⁶ and R³⁷ together are combined to form phenyl optionally substituted with 1, 2, or 3 R³¹ substituents.

In certain embodiments the cycle formed by combining R³⁶ and R³⁷ is not substituted.

In certain embodiments the cycle formed by combining R³⁶ and R³⁷ is substituted with 1 R³¹ substituent.

In certain embodiments the cycle formed by combining R³⁶ and R³⁷ is substituted with 2 R³¹ substituents.

In certain embodiments the cycle formed by combining R³⁶ and R³⁷ is substituted with 3 R³¹ substituents.

In certain embodiments R³⁶ is hydrogen.

In certain embodiments R³⁶ is halogen.

In certain embodiments R³⁶ is F.

In certain embodiments R³⁶ is Cl.

In certain embodiments R³⁶ is C₁₋₆-alkyl.

In certain embodiments R³⁶ is cyano.

In certain embodiments R³⁶ is C₁₋₆-alkoxy.

In certain embodiments R³⁶ is halo-C₁₋₆-alkoxy.

In certain embodiments R³⁶ is C₃₋₈-cycloalkyl.

In certain embodiments R³⁶ is halo-C₃₋₈-cycloalkyl.

In certain embodiments R³⁶ is selected from hydrogen, halogen, C₁₋₆-alkoxy, and C₁₋₆-alkyl.

In certain embodiments R³⁶ is selected from hydrogen, F, Cl, methoxy, and methyl.

In certain embodiments R³⁷ is hydrogen.

In certain embodiments R³⁷ is halogen.

In certain embodiments R³⁷ is F.

In certain embodiments R³⁷ is Cl.

In certain embodiments R³⁷ is C₁₋₆-alkyl.

In certain embodiments R³⁷ is cyano.

In certain embodiments R³⁷ is C₁₋₆-alkoxy.

In certain embodiments R³⁷ is halo-C₁₋₆-alkoxy.

In certain embodiments R³⁷ is C₃₋₈-cycloalkyl.

In certain embodiments R³⁷ is halo-C₃₋₈-cycloalkyl.

In certain embodiments R³⁷ is selected from hydrogen, halogen, C₁₋₆-alkoxy, and C₁₋₆-alkyl.

In certain embodiments R³⁷ is selected from hydrogen, F, Cl, methoxy, and methyl.

### Embodiments of R⁴²

In certain embodiments at least one R⁴² is halogen.

In certain embodiments at least one R⁴² is F.

In certain embodiments at least one R⁴² is Cl.

In certain embodiments at least one R⁴² is C₁₋₆-alkyl.

In certain embodiments at least one R⁴² is halo-C₁₋₆-alkyl.

In certain embodiments R⁴² is hydrogen.

In certain embodiments R⁴² is halogen.

In certain embodiments R⁴² is F.

In certain embodiments R⁴² is Cl.

In certain embodiments R⁴² is C₁₋₆-alkyl.

In certain embodiments R⁴² is cyano.

In certain embodiments R⁴² is C₁₋₆-alkoxy

In certain embodiments R⁴² is halo-C₁₋₆-alkoxy.

In certain embodiments R⁴² is C₃₋₈-cycloalkyl.

In certain embodiments R⁴² is halo-C₃₋₈-cycloalkyl.

In certain embodiments R⁴² is selected from hydrogen, halogen, C₁₋₆-alkoxy, and C₁₋₆-alkyl.

In certain embodiments R⁴² is selected from hydrogen, F, Cl, methoxy, and methyl.

### Embodiments of Ring G

In certain embodiments Ring G is a 5-membered heteroaryl ring optionally substituted with 1 or 2 R⁴² substituents.

In certain embodiments Ring G is a 6-membered heteroaryl ring optionally substituted with 1 or 2 R⁴² substituents.

In certain embodiments Ring G is selected from:

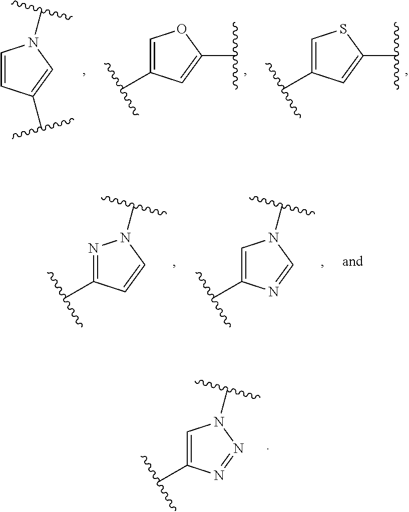 [SMILES: *c1ccn(*)c1 ; *c1coc(*)c1 ; *c1csc(*)c1 ; *c1ccn(*)n1 ; *c1cn(*)cn1 ; *c1cn(*)nn1]

### Embodiments of EGFR Targeting Ligand

In certain embodiments the compound for use in the methods of treatment described herein is selected from:

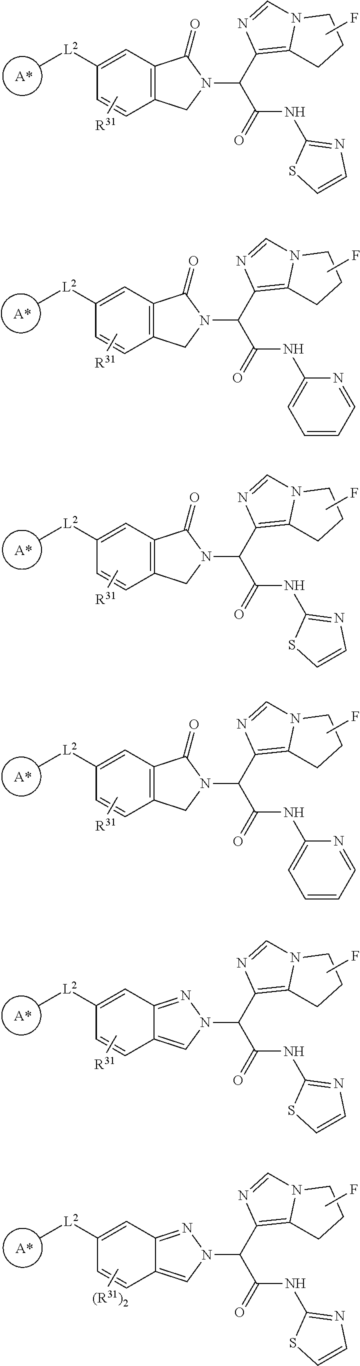

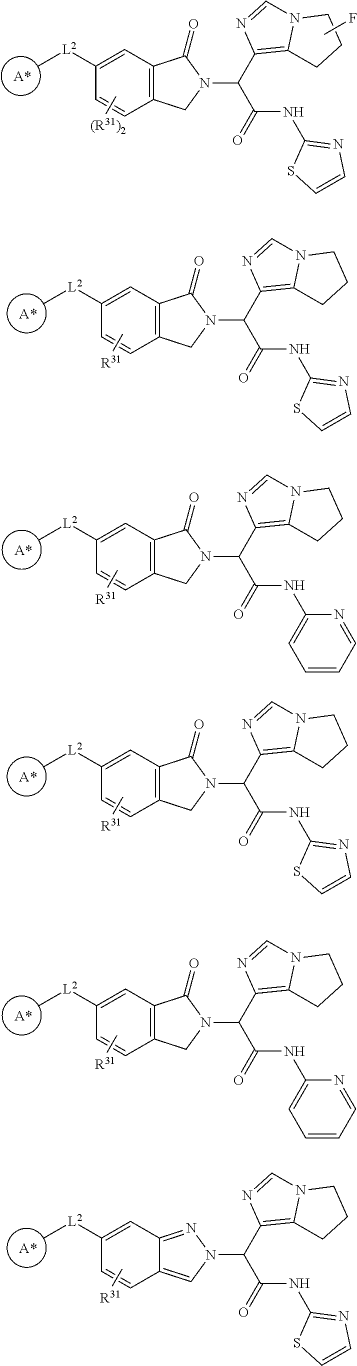

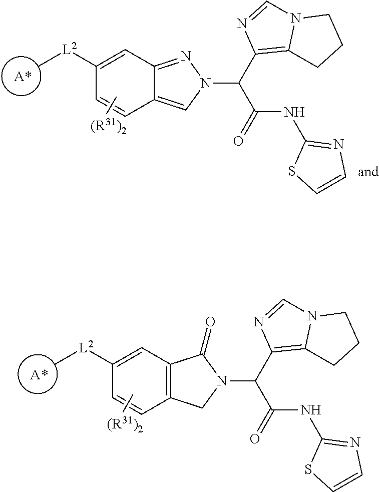

In certain embodiments the compound for use in the methods of treatment described herein is selected from:

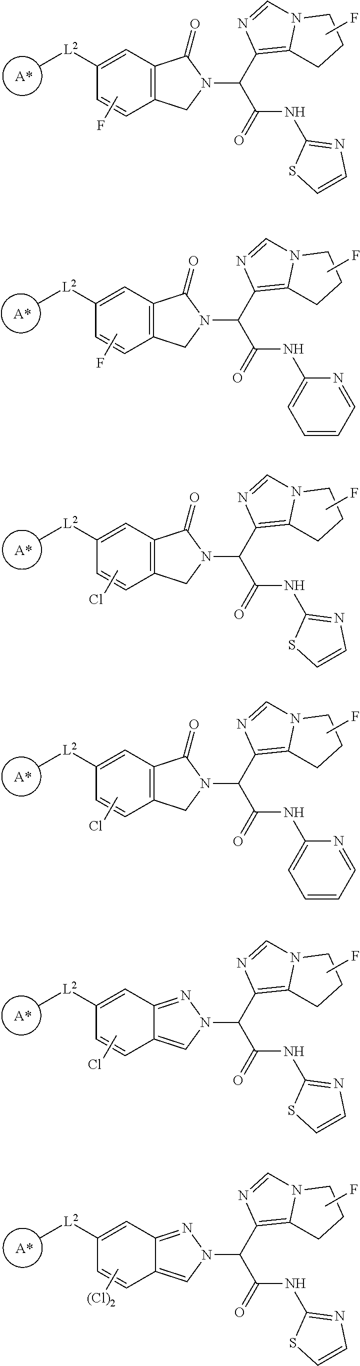

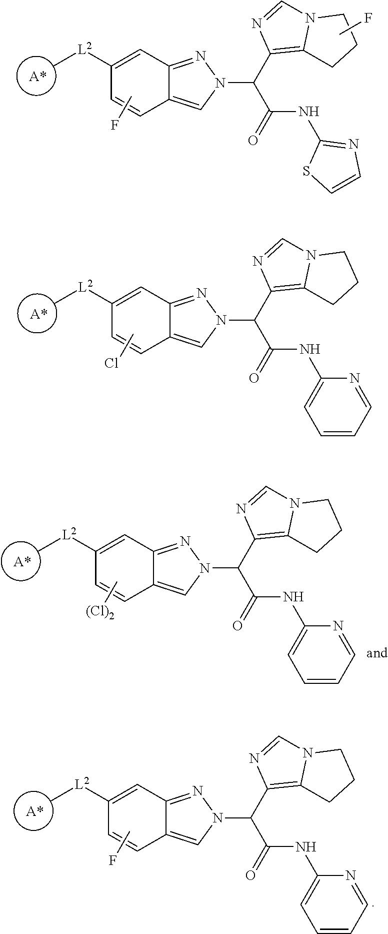

In certain embodiments the compound for use in the methods of treatment described herein is selected from:

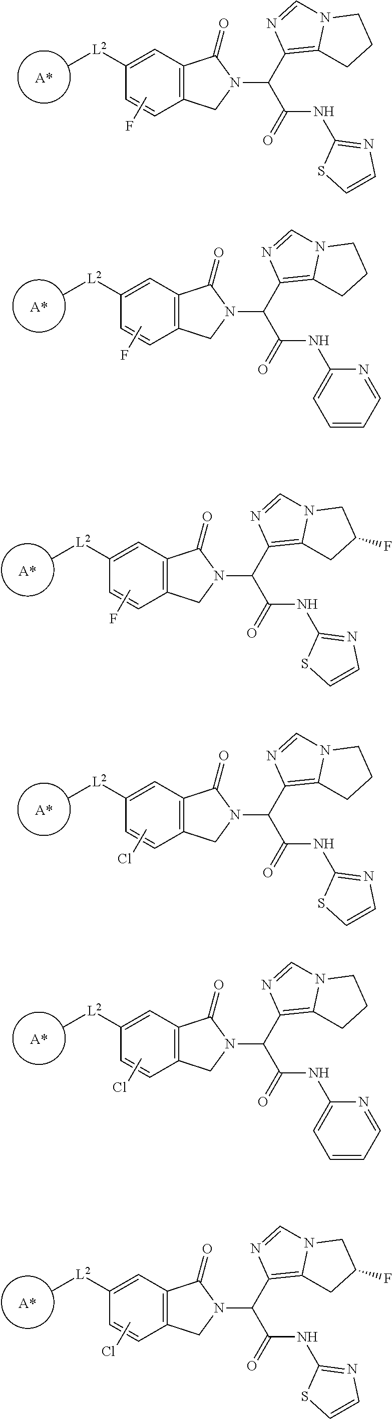

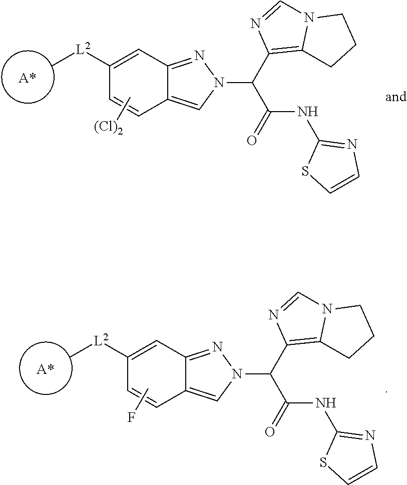

In certain embodiments the compound for use in the methods of treatment described herein is selected from:

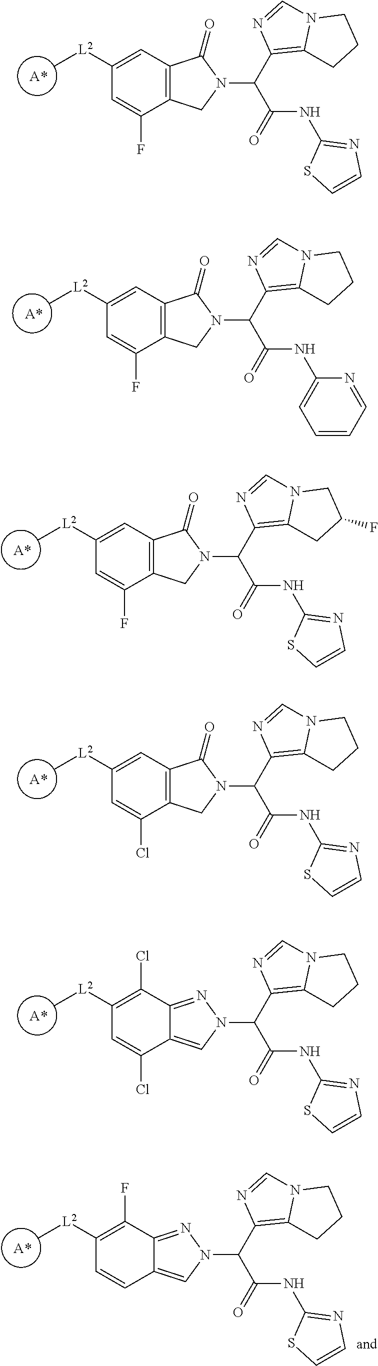

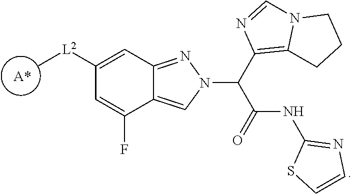

In certain embodiments the compound for use in the methods of treatment described herein is selected from:

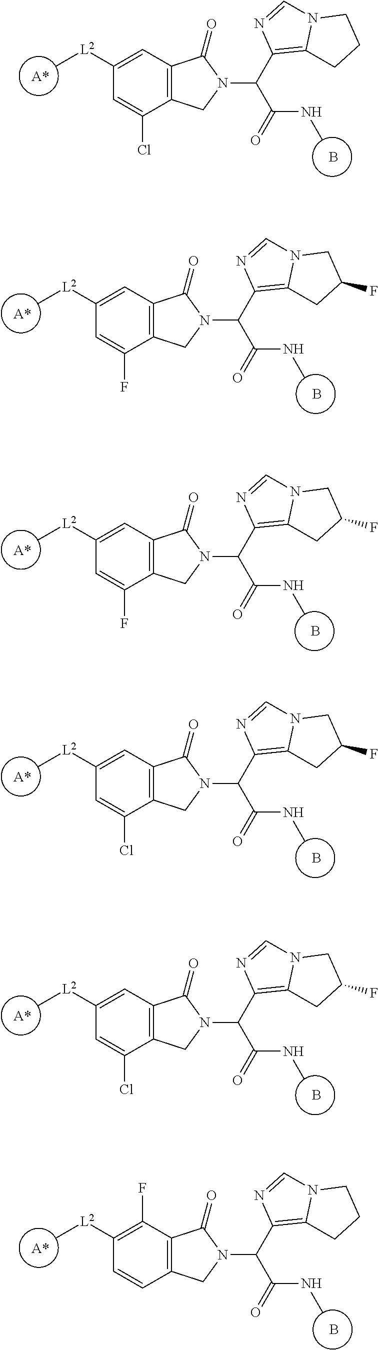

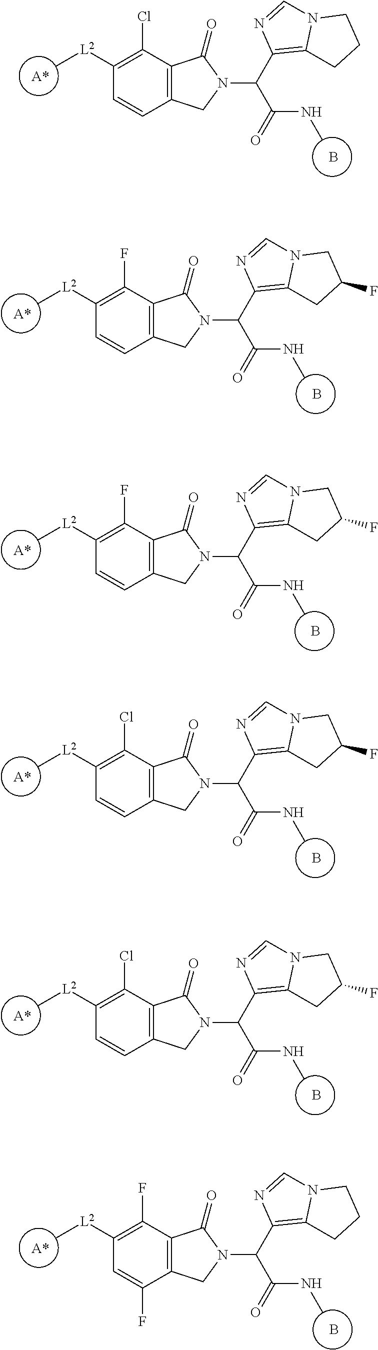

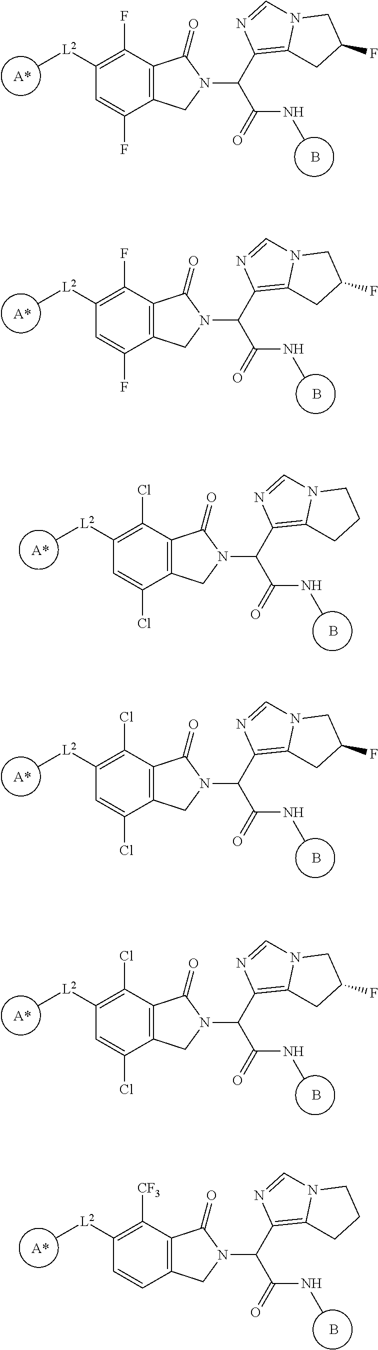

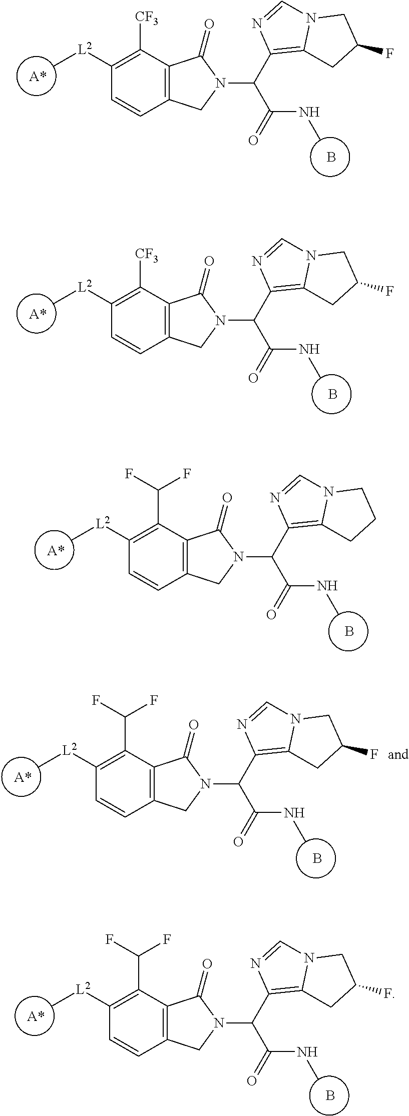

In certain embodiments the compound for use in the methods of treatment described herein is selected from:

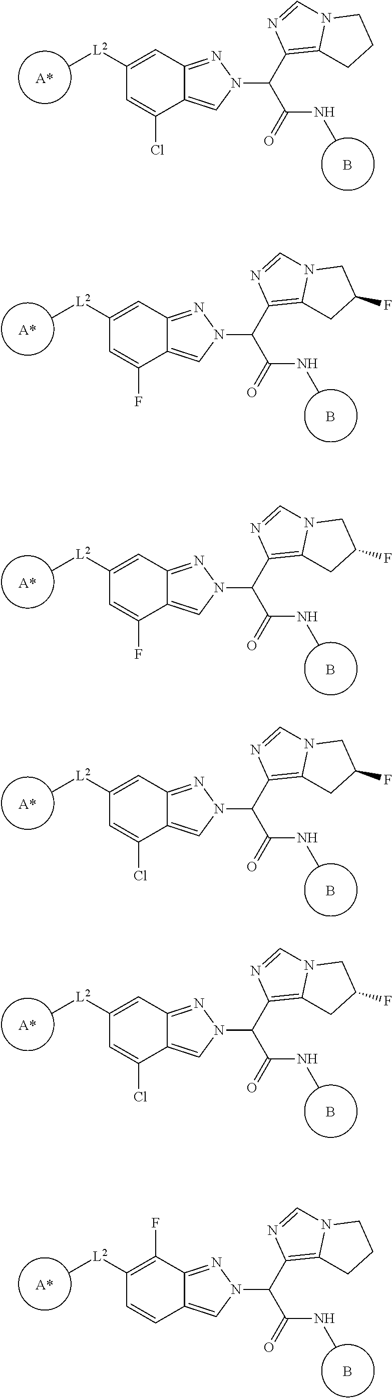

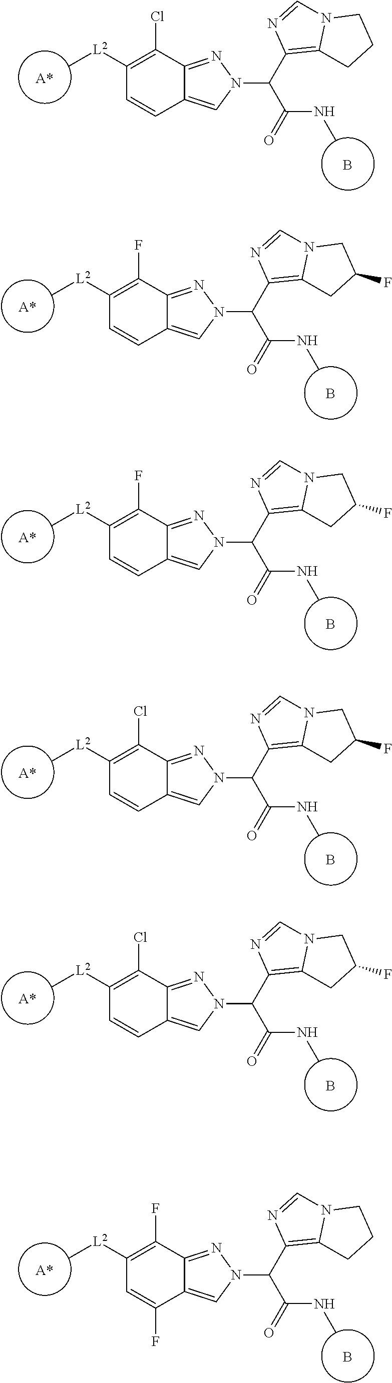

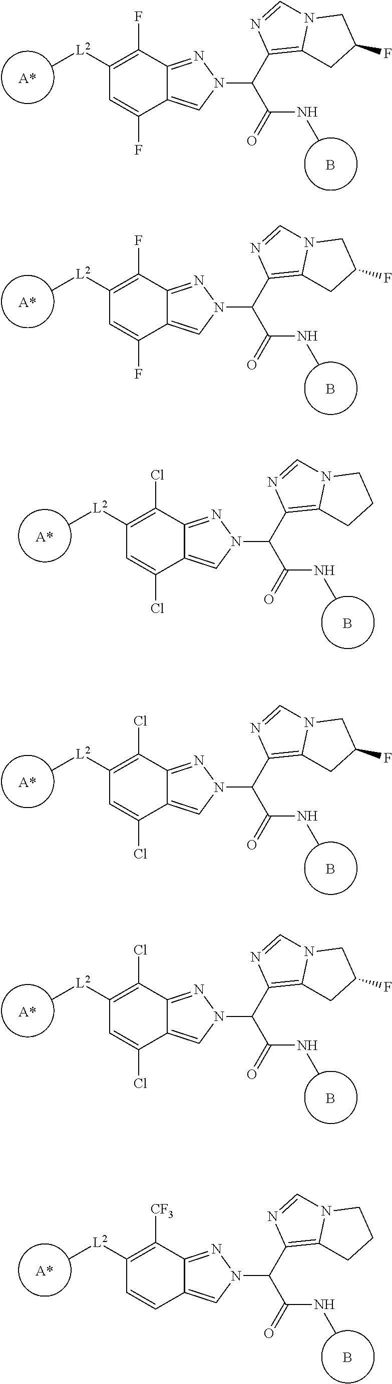

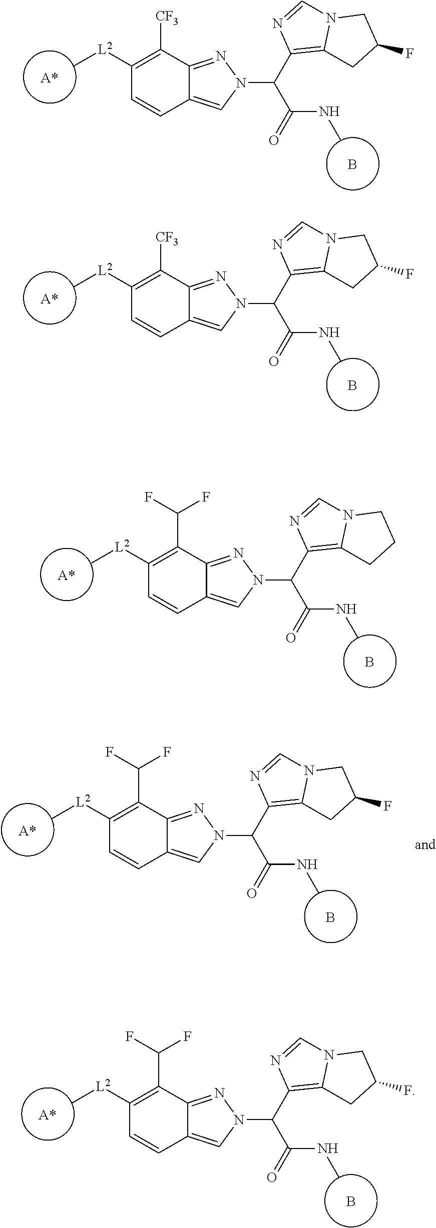

### Compounds of Formula III

In certain embodiments the compound for use in the methods of treatment described herein is selected from:

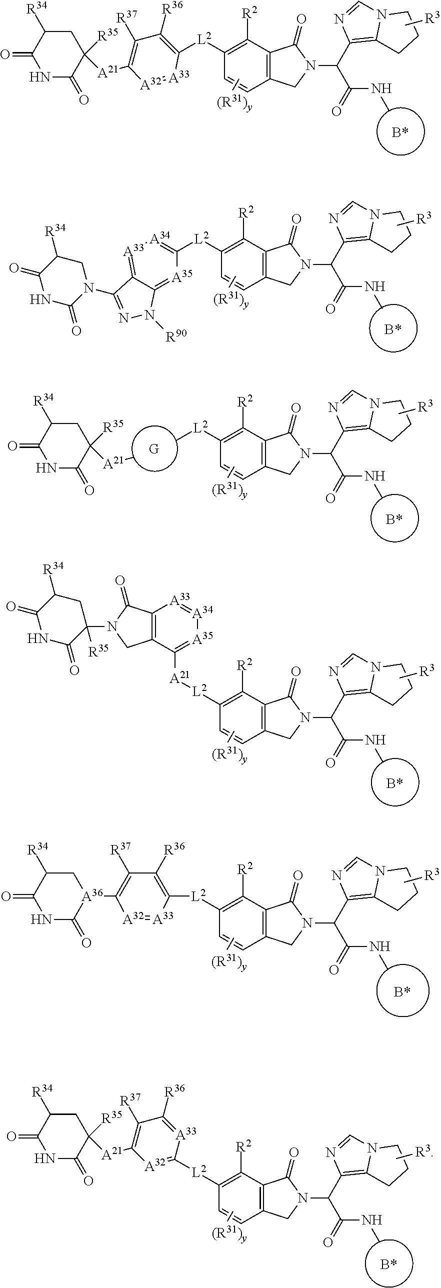

In certain embodiments the compound for use in the methods of treatment described herein is selected from:

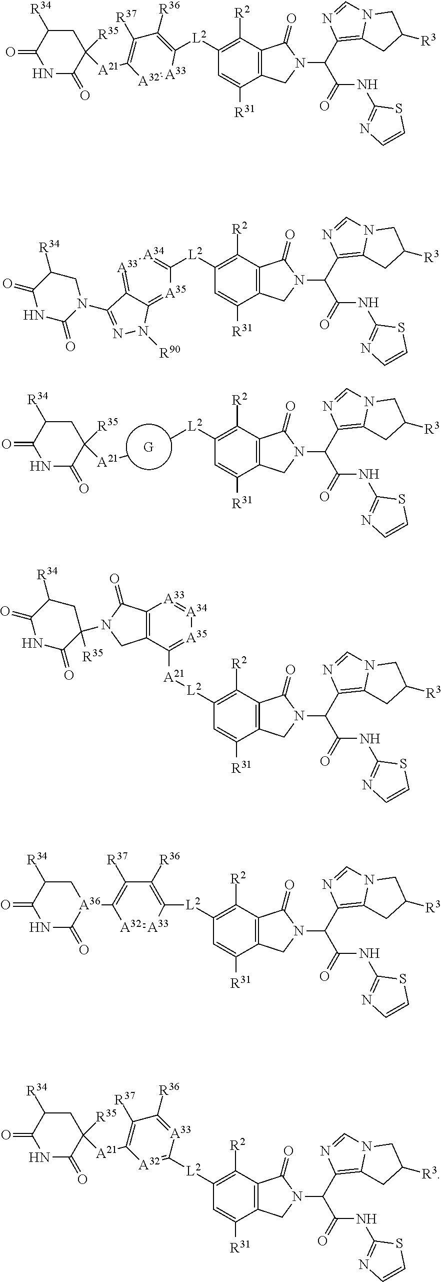

### Compounds of Formula IV

In certain embodiments the compound for use in the methods of treatment described herein is selected from:

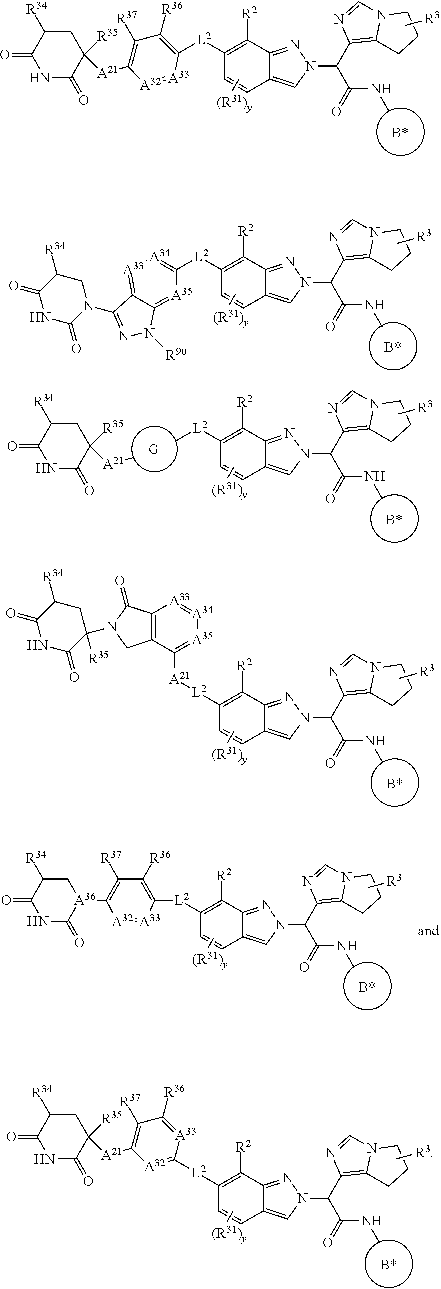

### III. Additional Compounds for Use in the Present Invention

In certain embodiments the compound for use in the methods of treatment described herein is selected from:

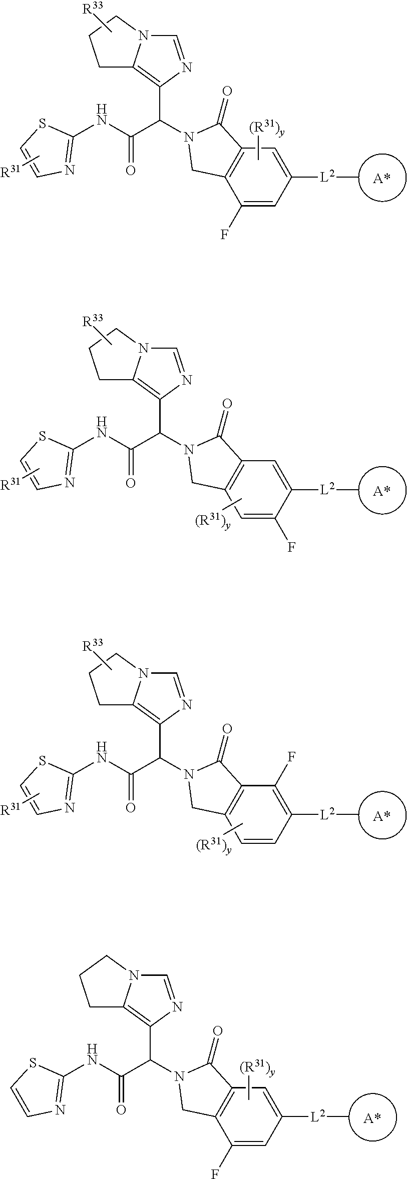

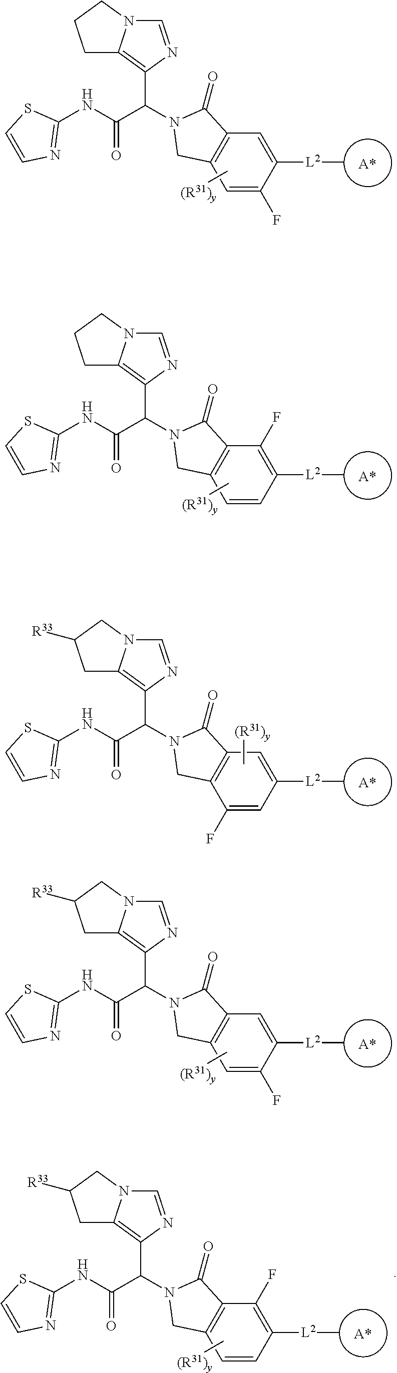

In certain embodiments the compound for use in the methods of treatment described herein is selected from:

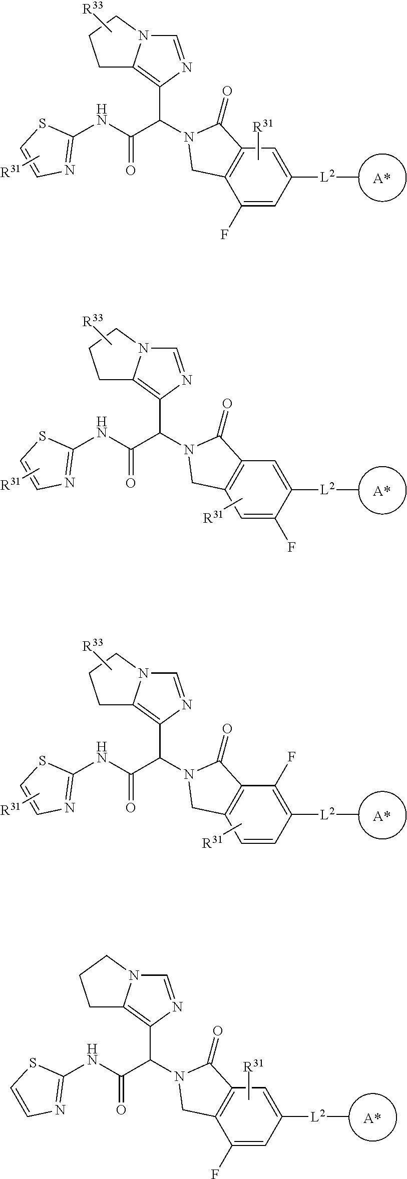

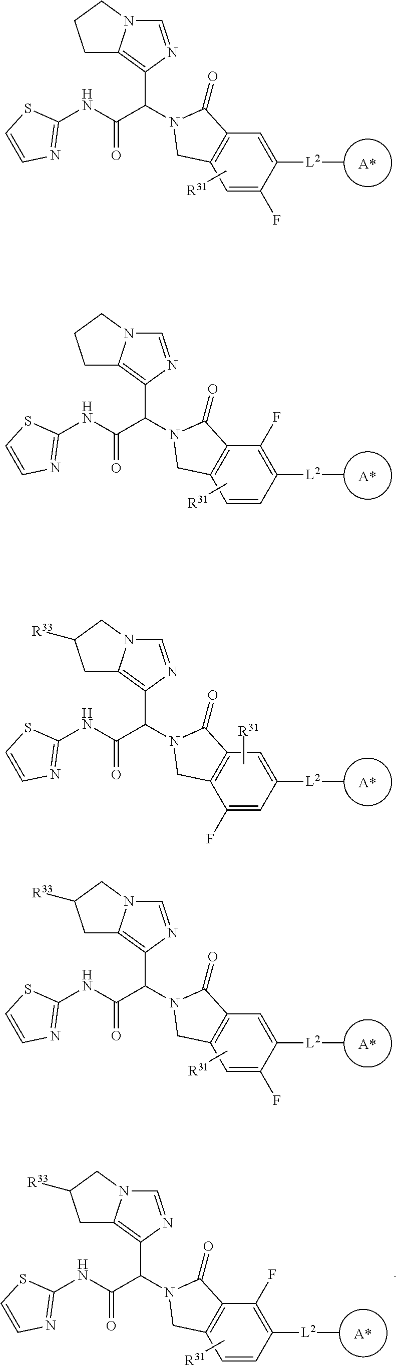

In certain embodiments the compound for use in the methods of treatment described herein is selected from:

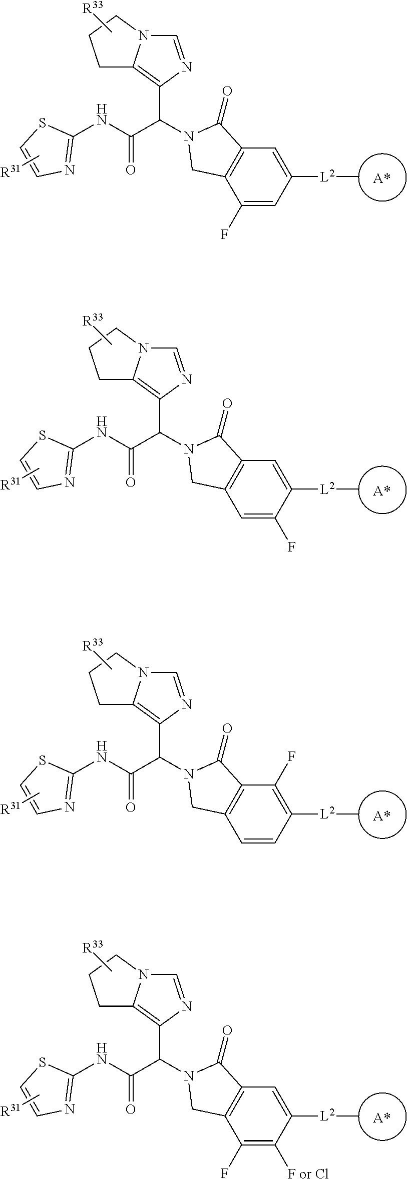

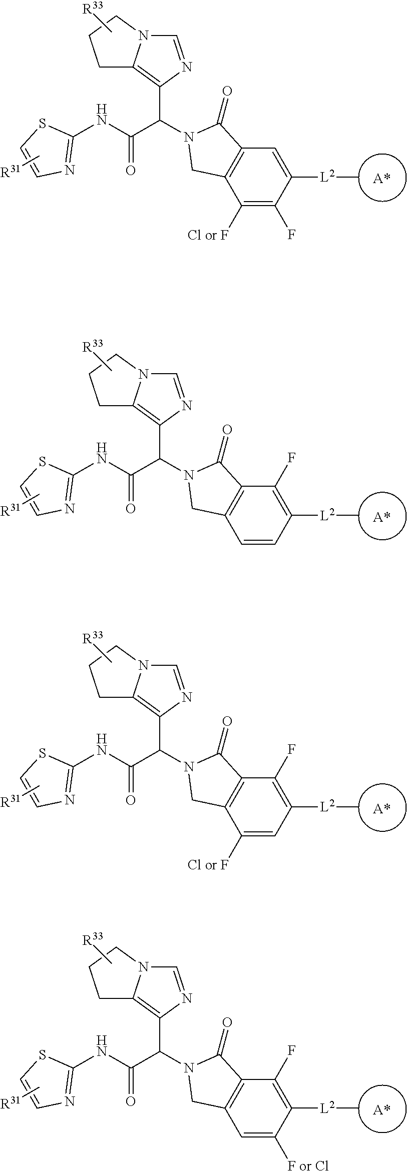

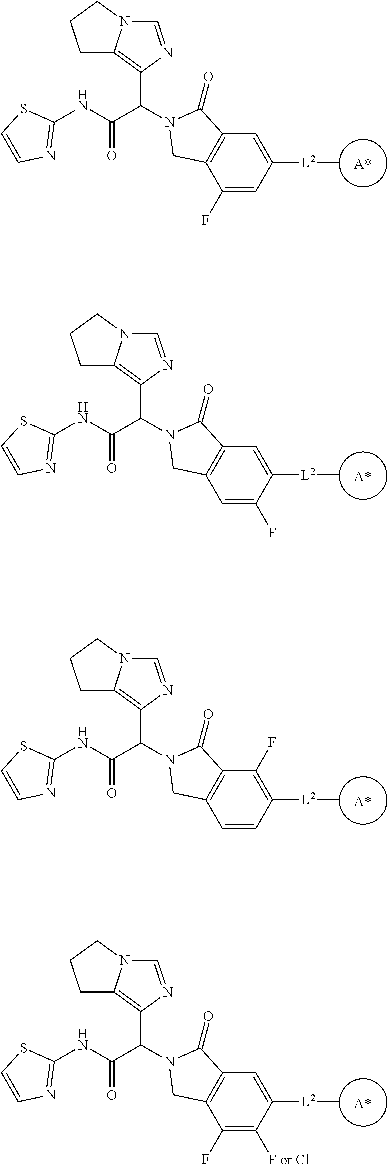

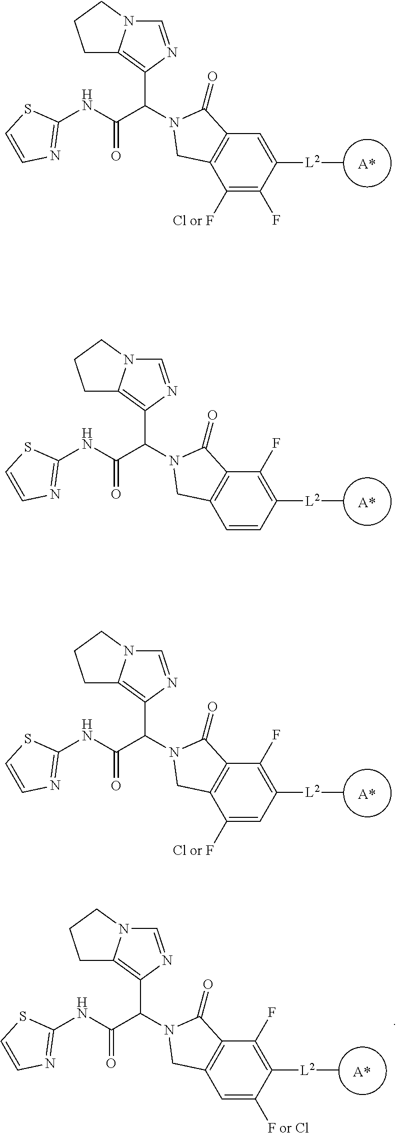

In certain embodiments the compound for use in the methods of treatment described herein is selected from:

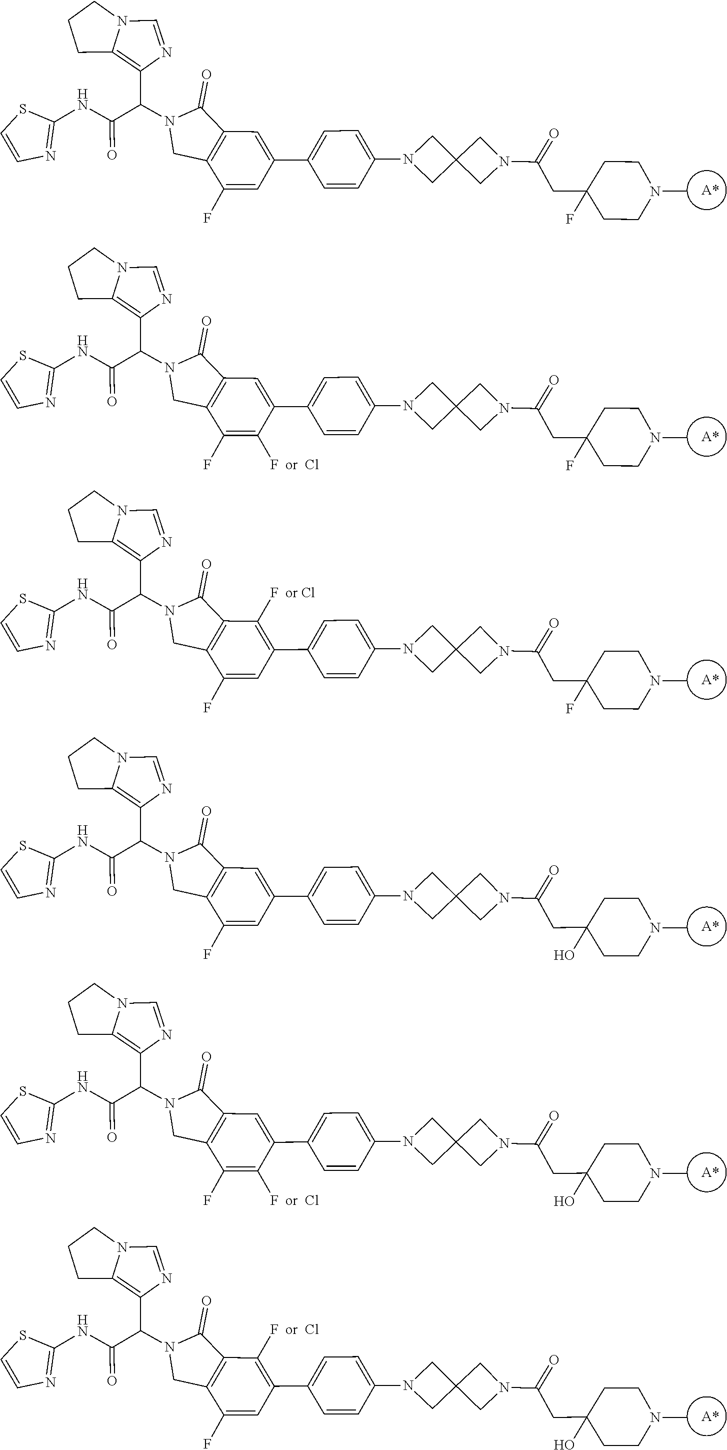

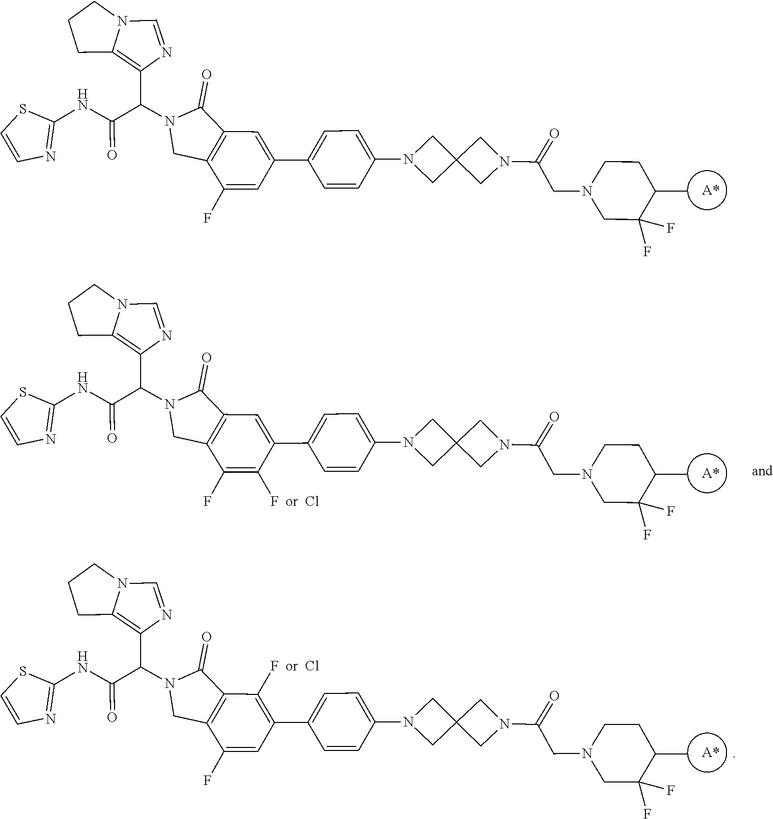

In certain embodiments the compound for use in the methods of treatment described herein is selected from:

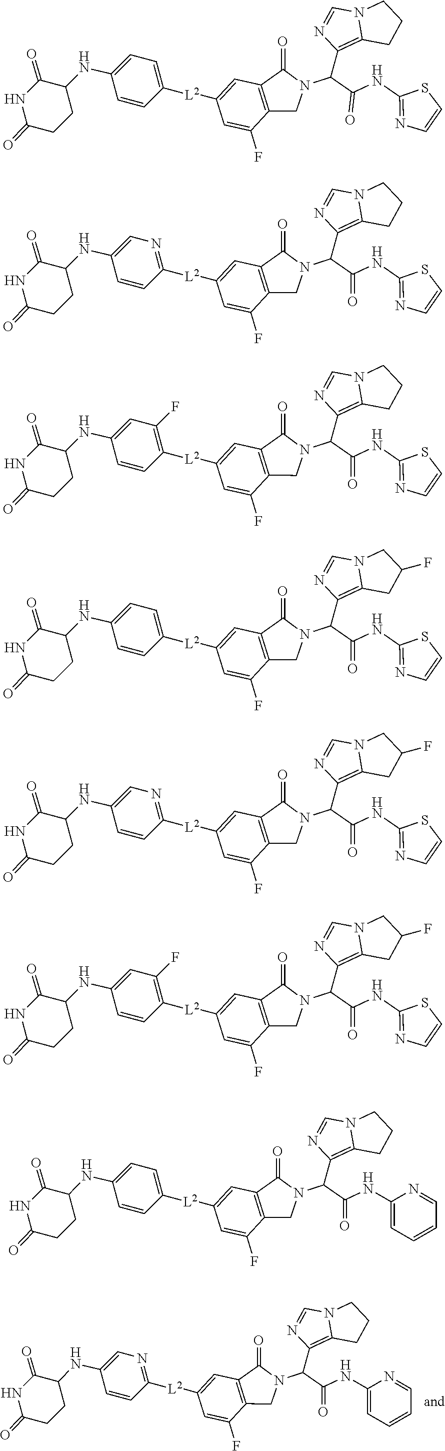

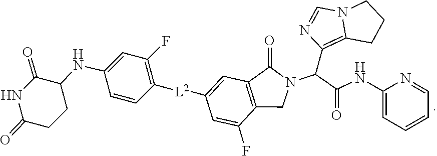

In certain embodiments the compound for use in the methods of treatment described herein is selected from:

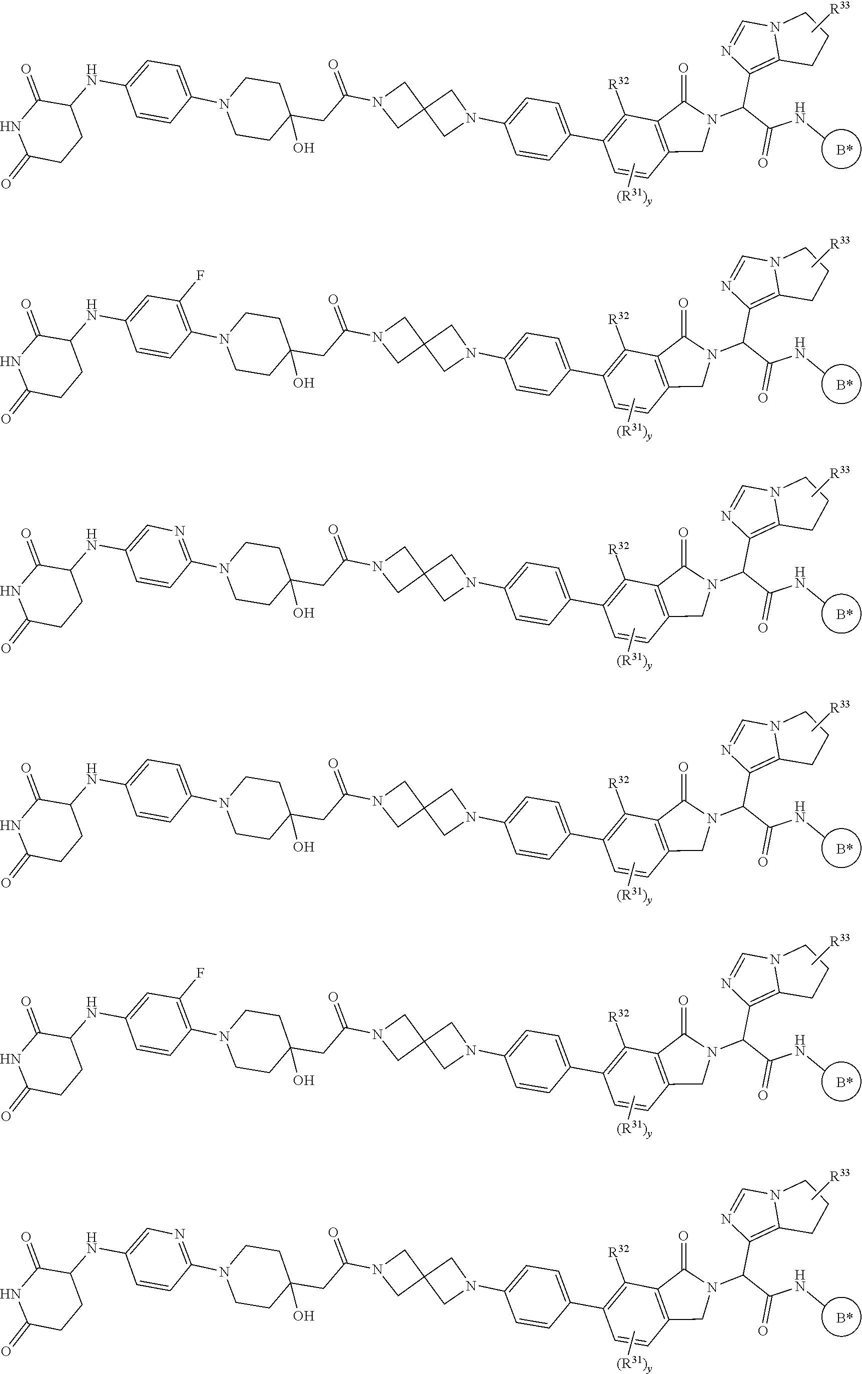

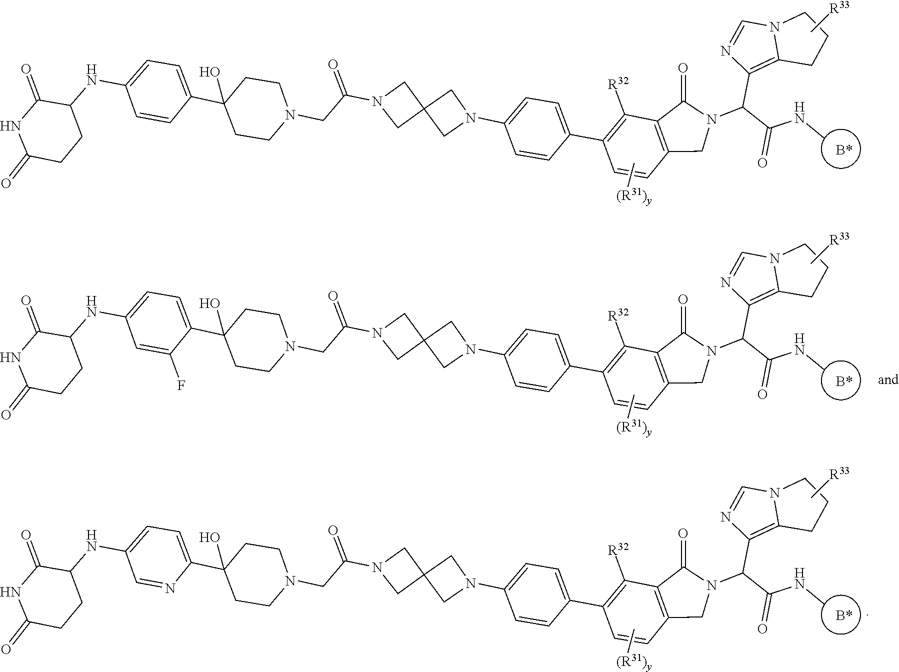

In certain embodiments the compound for use in the methods of treatment described herein is selected from:

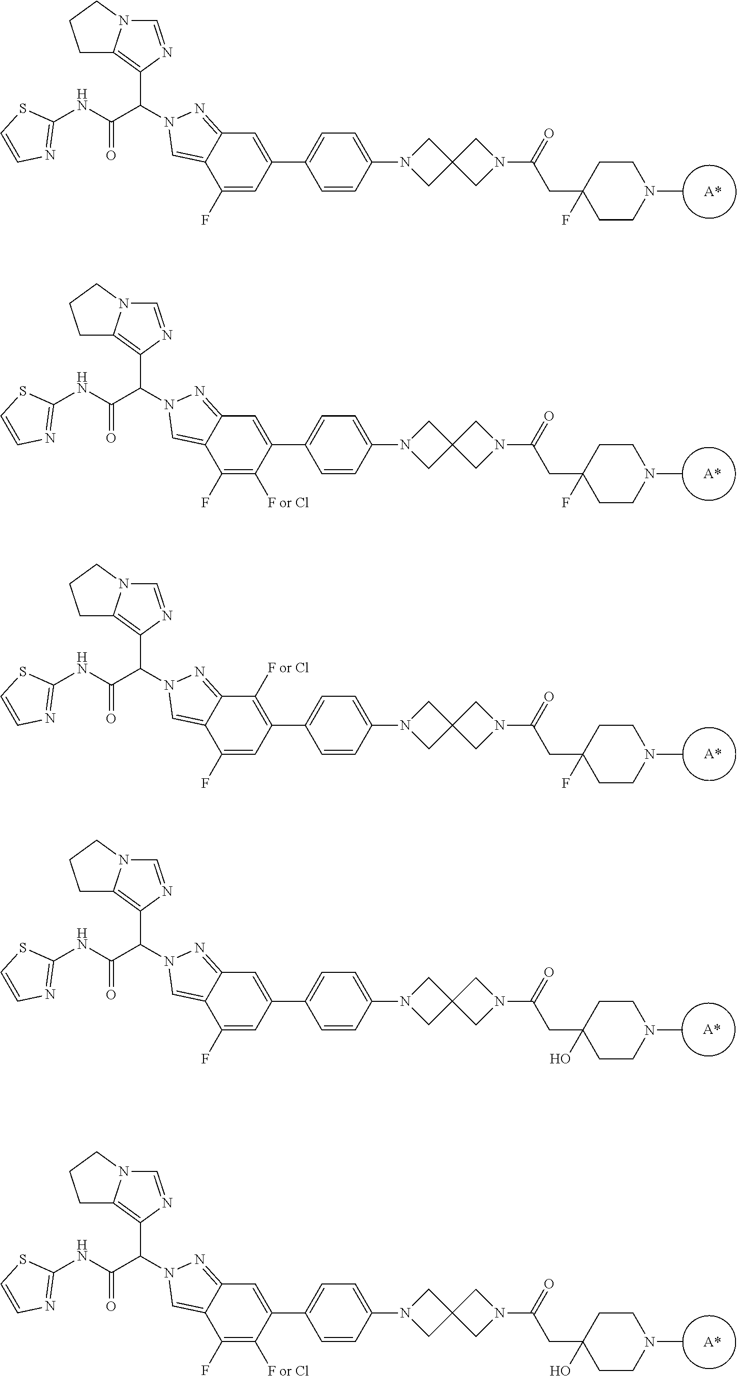

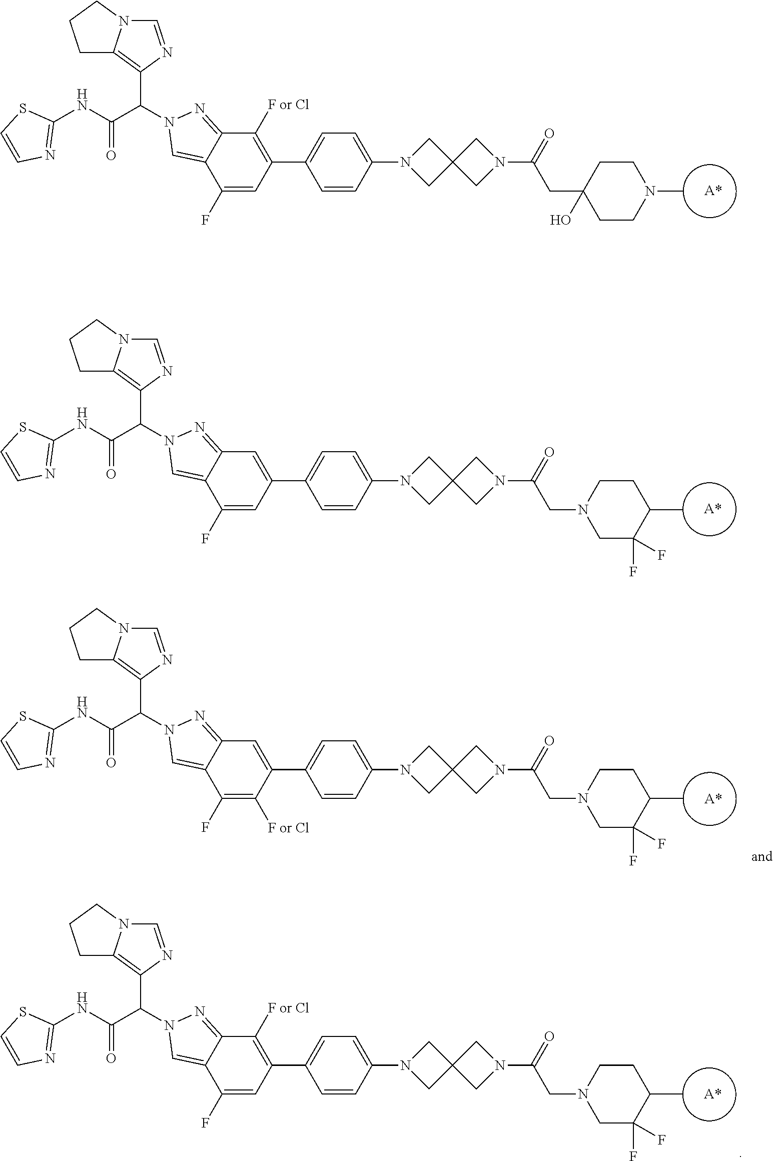

In certain embodiments the compound for use in the methods of treatment described herein is selected from:

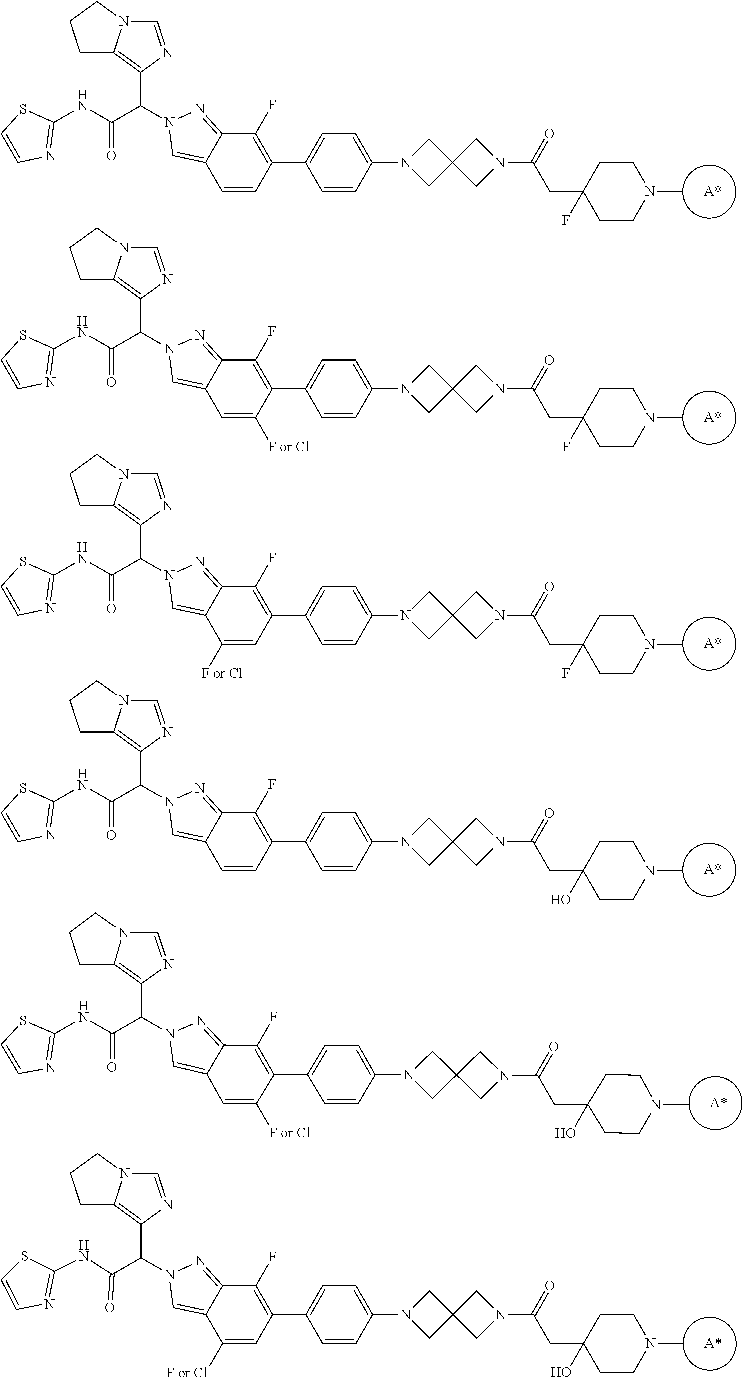

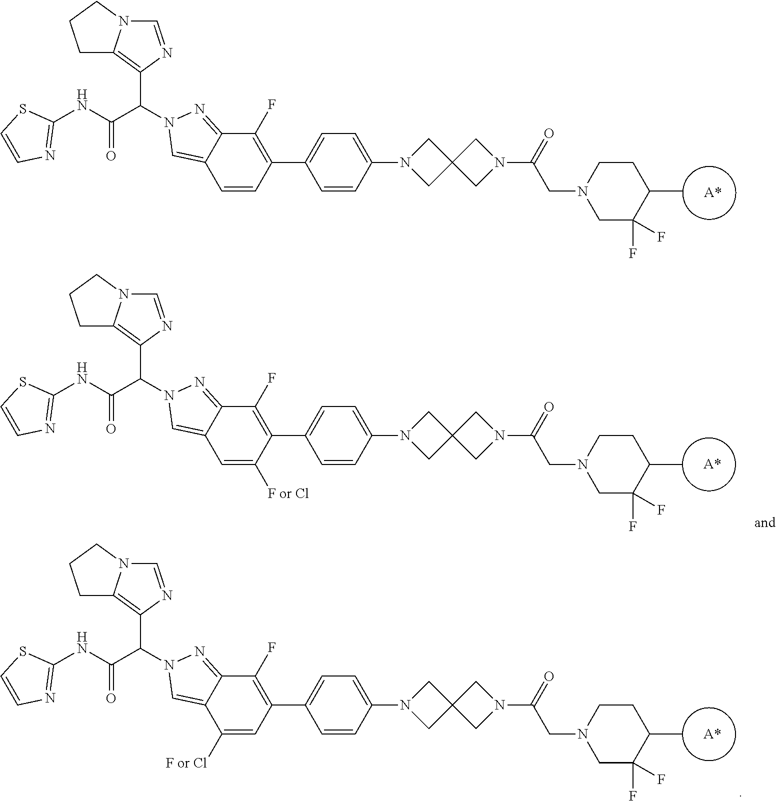

In certain embodiments the compound for use in the methods of treatment described herein is selected from:

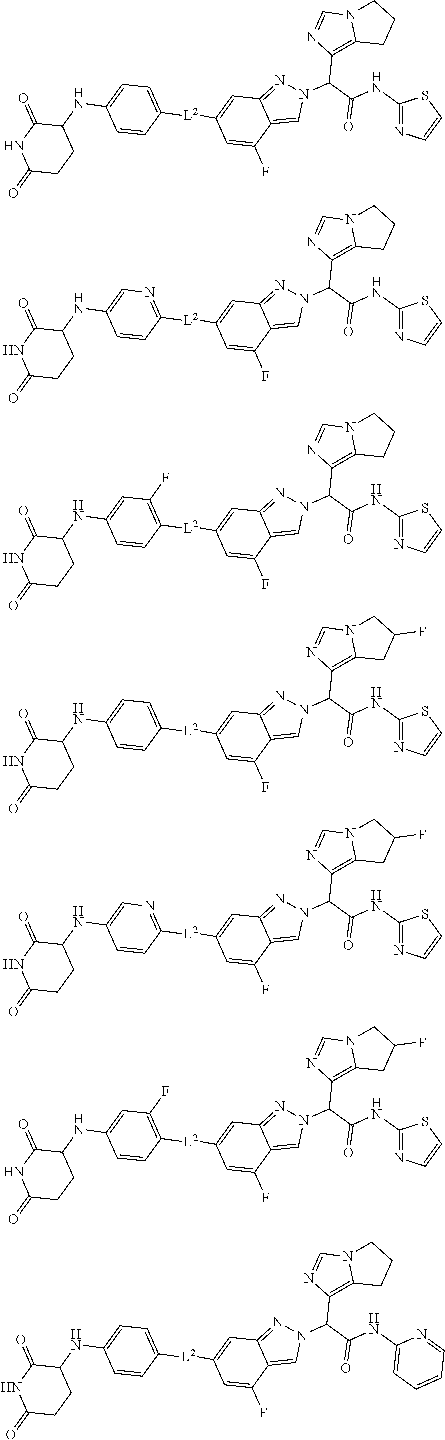

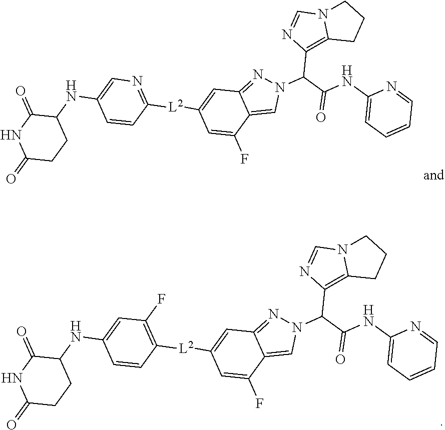

In certain embodiments the compound for use in the methods of treatment described herein is selected from:

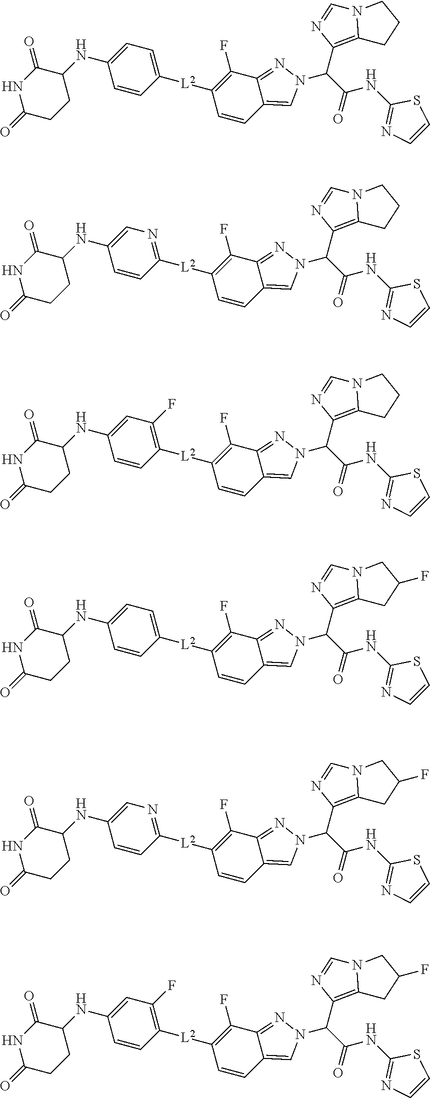

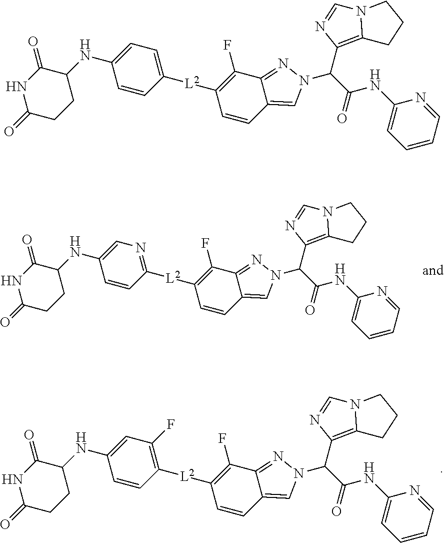

In certain embodiments the compound for use in the methods of treatment described herein is selected from:

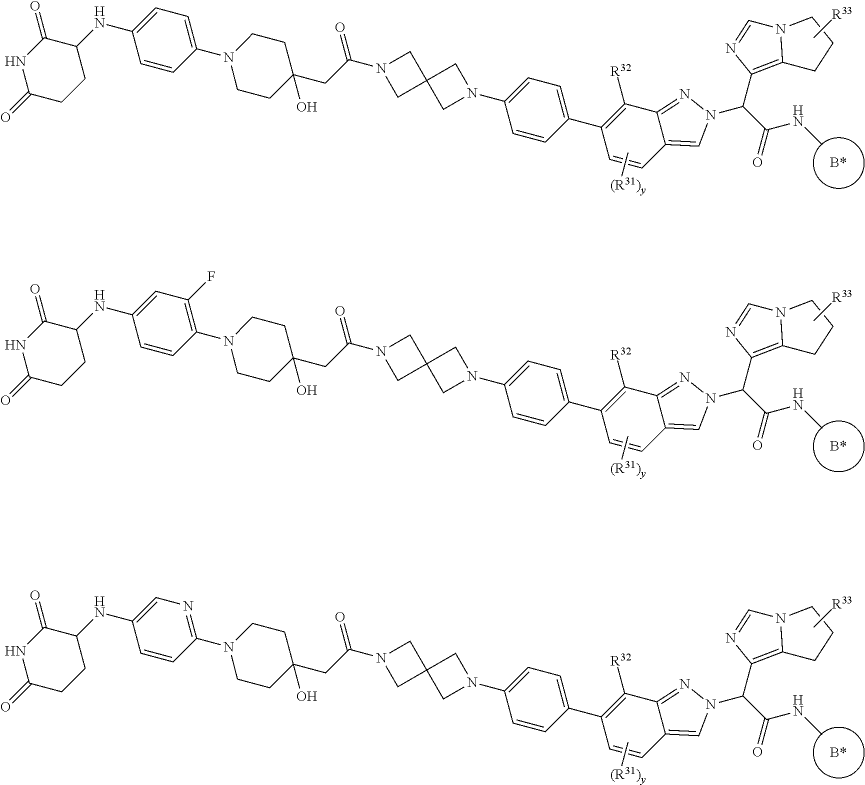

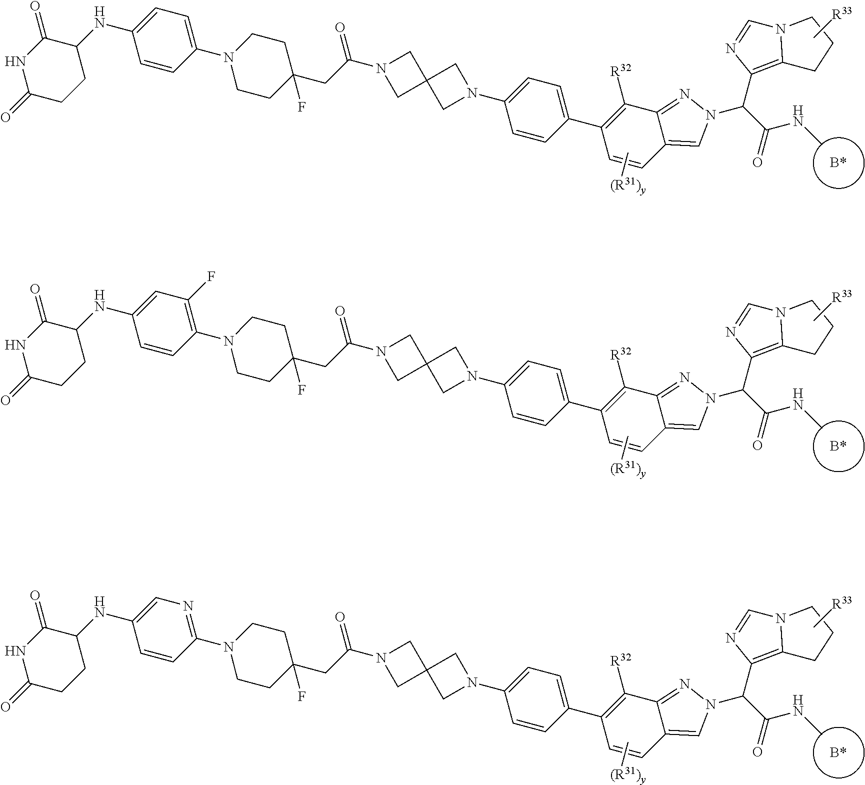

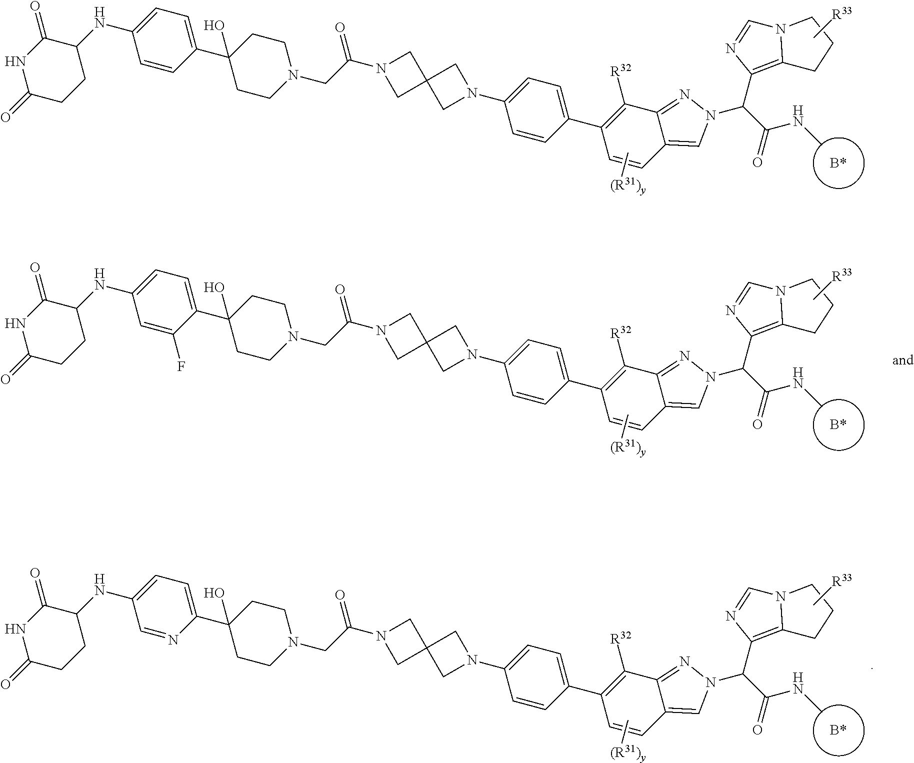

In certain embodiments the compound for use in the methods of treatment described herein is selected from:

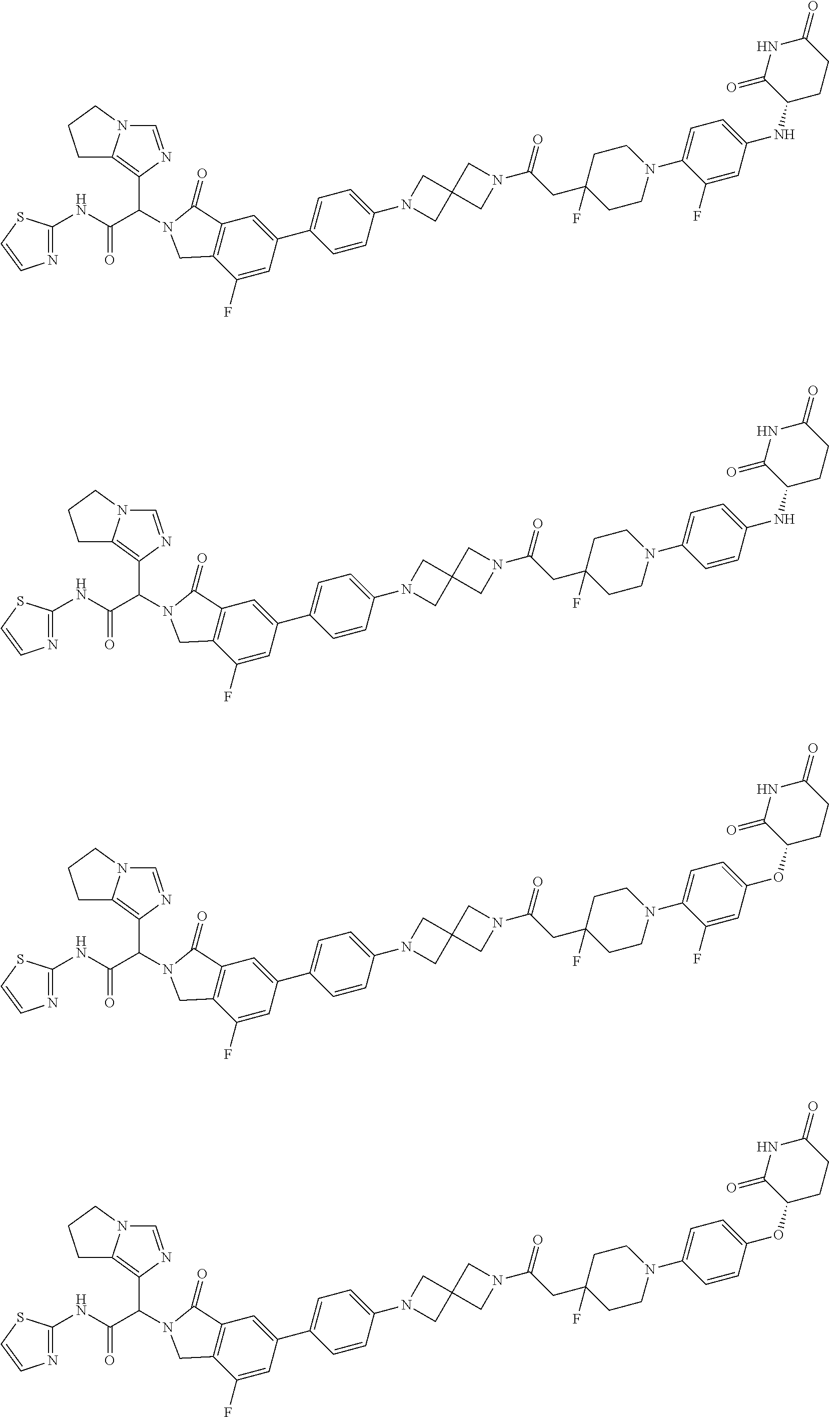

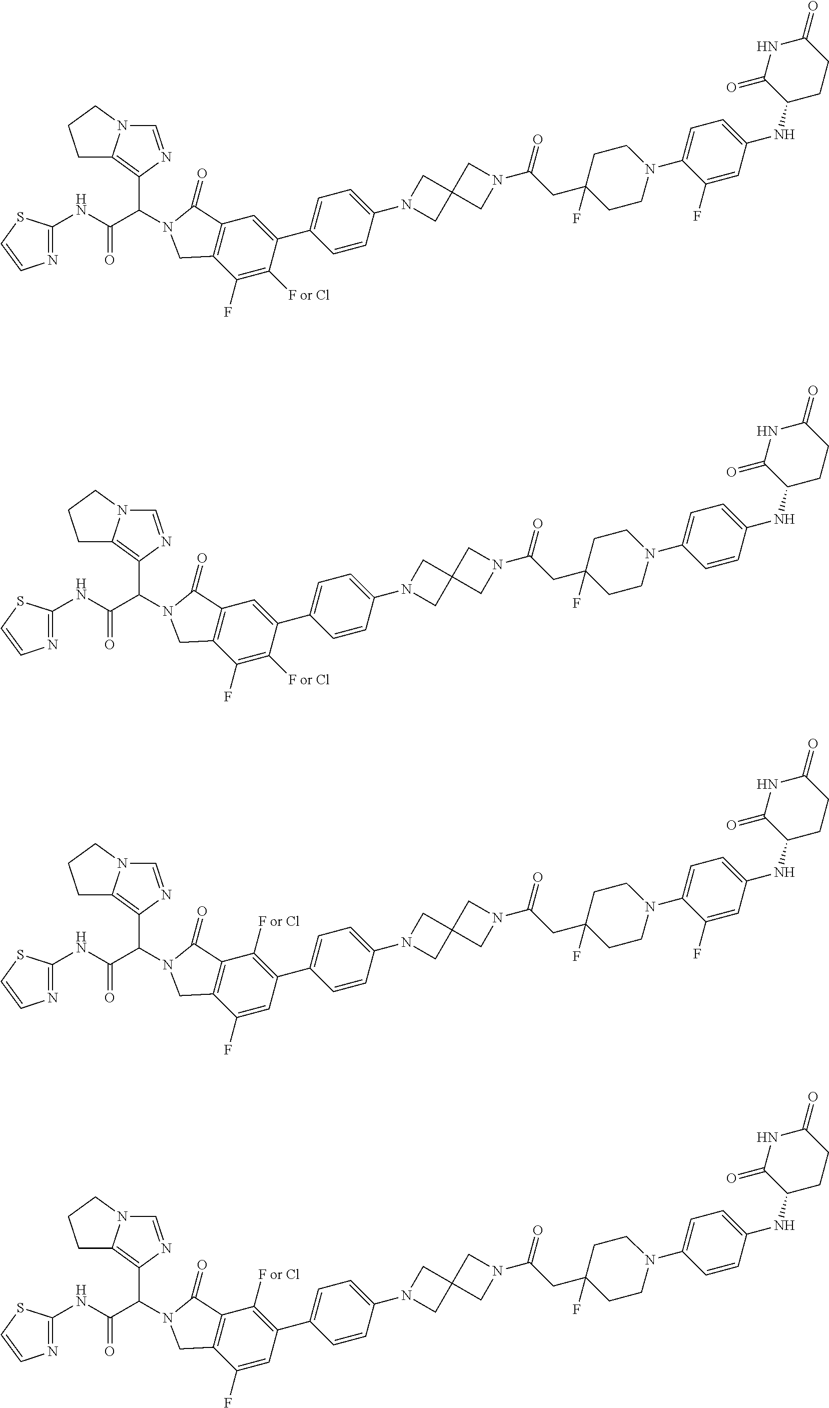

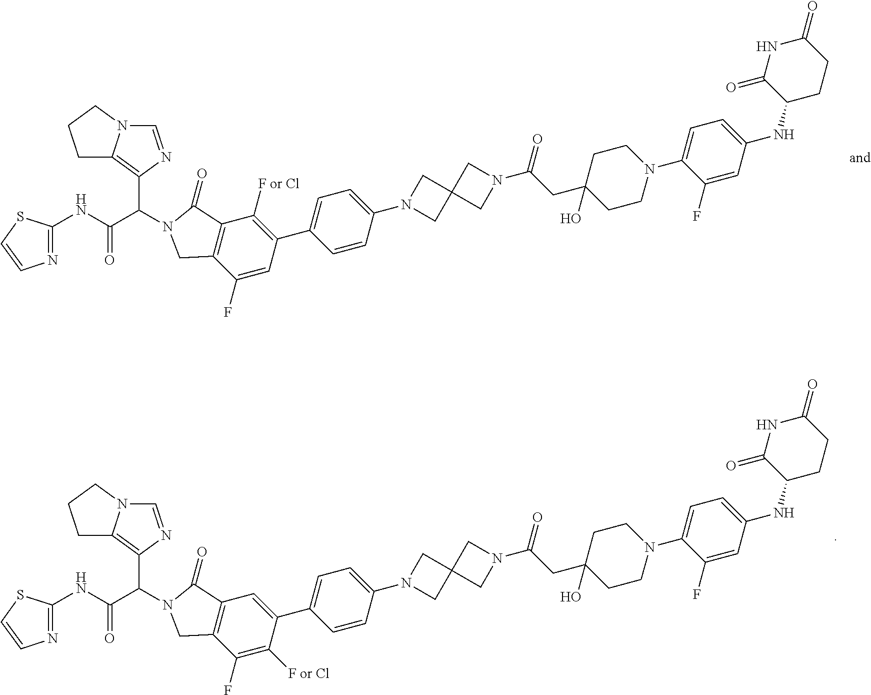

In certain embodiments the compound for use in the methods of treatment described herein is selected from:

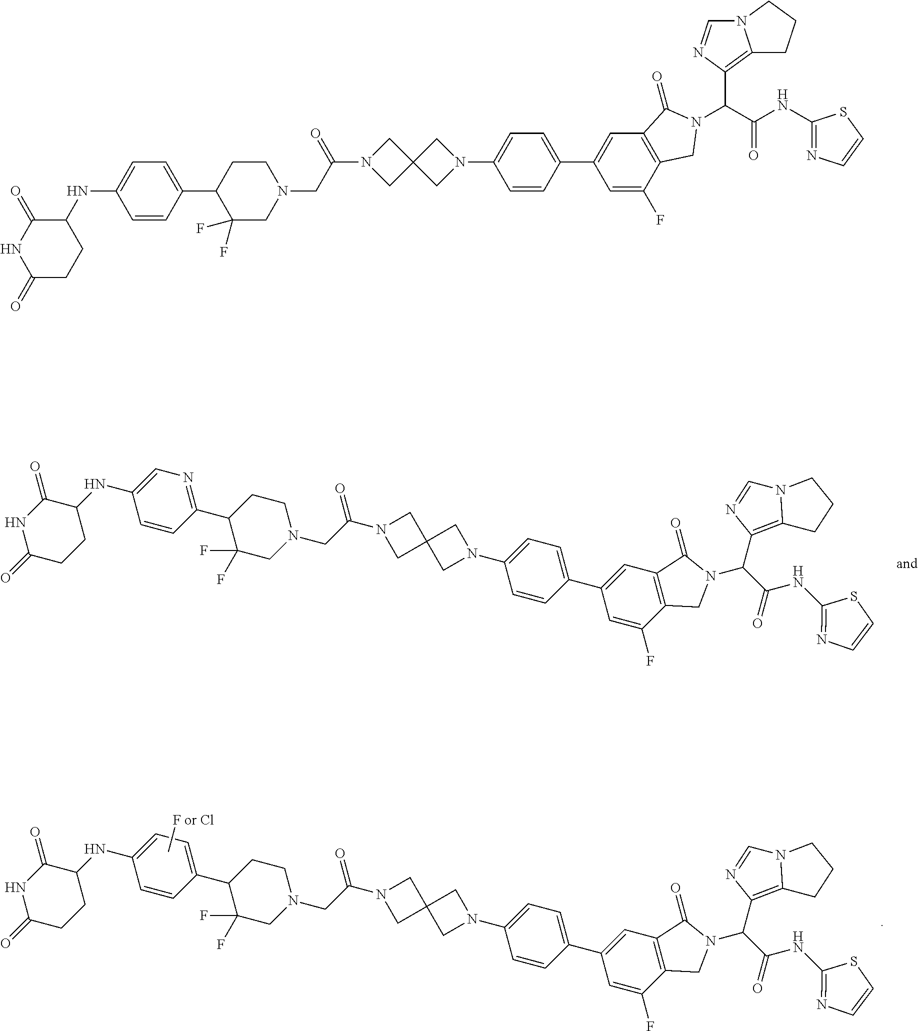

In certain embodiments the compound for use in the methods of treatment described herein is selected from:

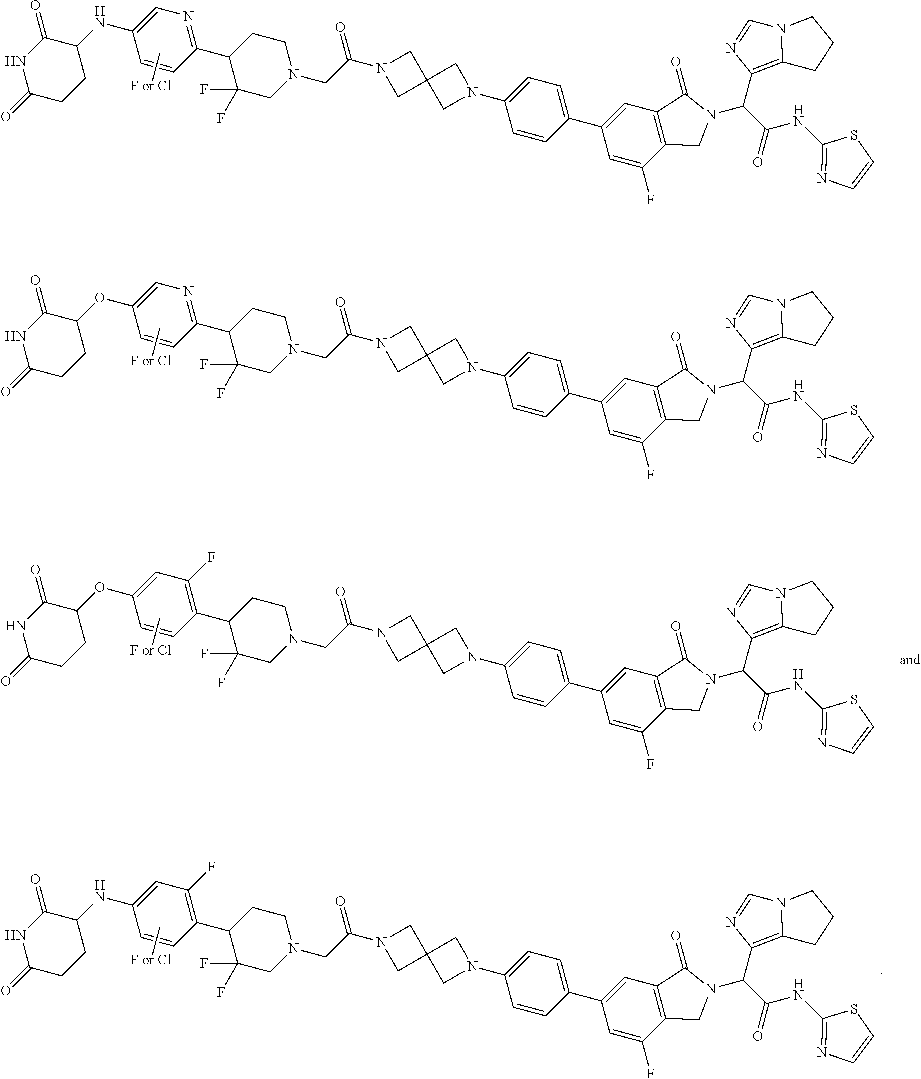

In certain embodiments the compound for use in the methods of treatment described herein is selected from:

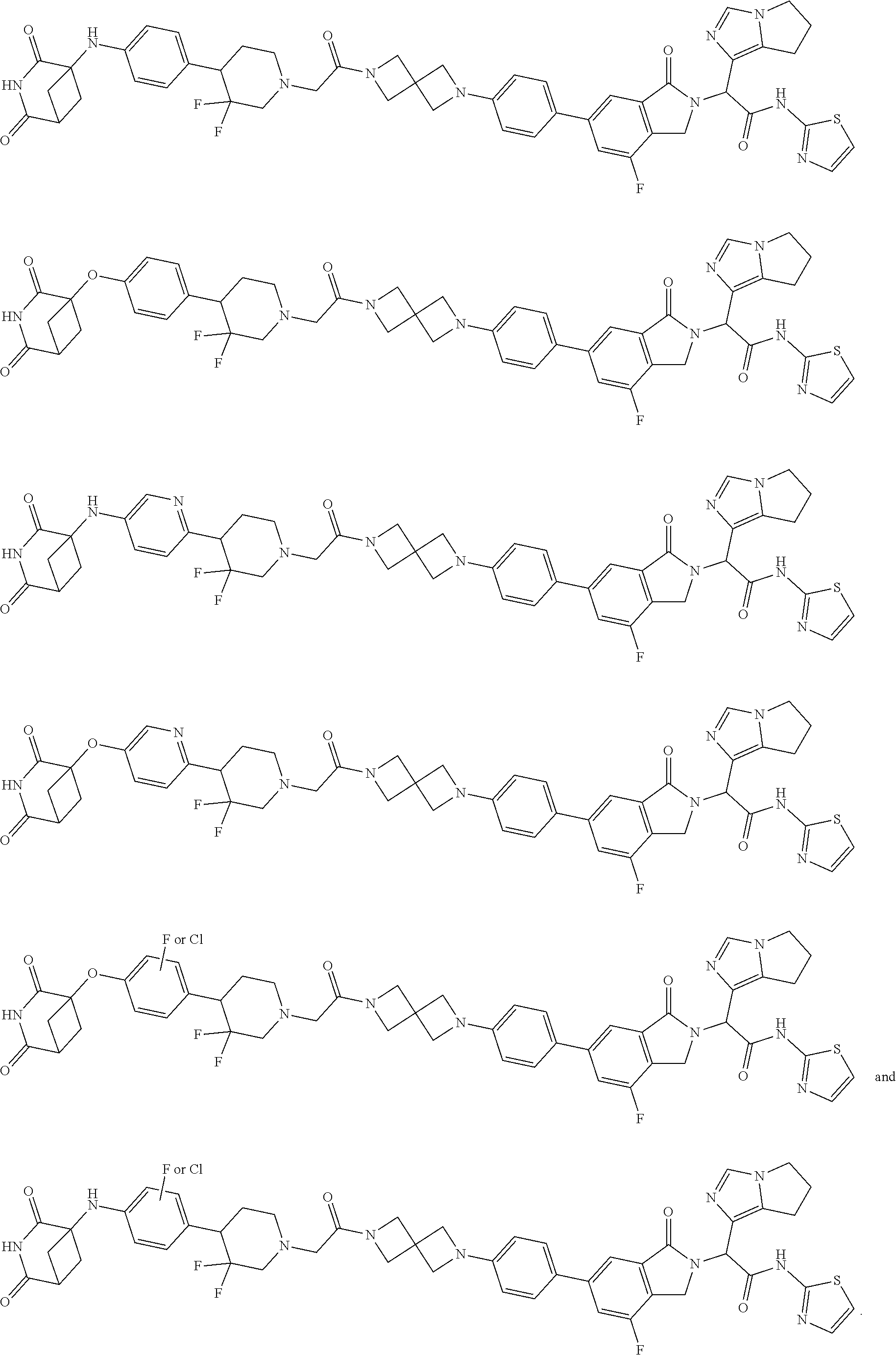

In certain embodiments the compound for use in the methods of treatment described herein is selected from:

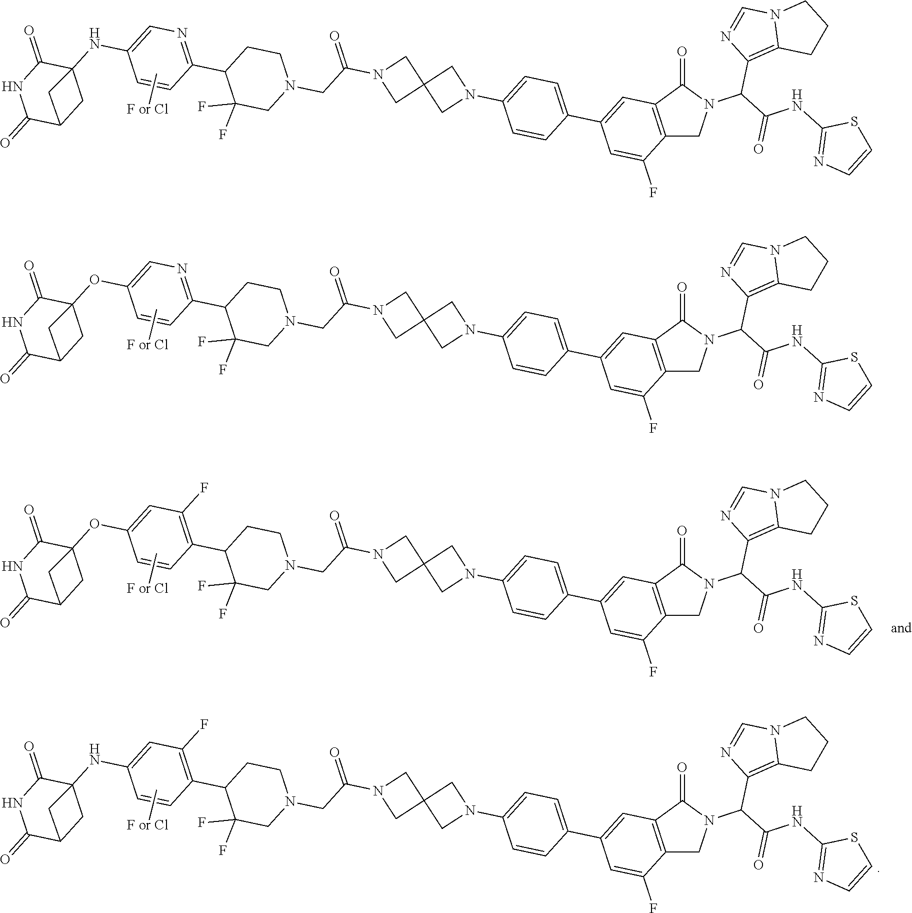

In certain embodiments the compound for use in the methods of treatment described herein is selected from:

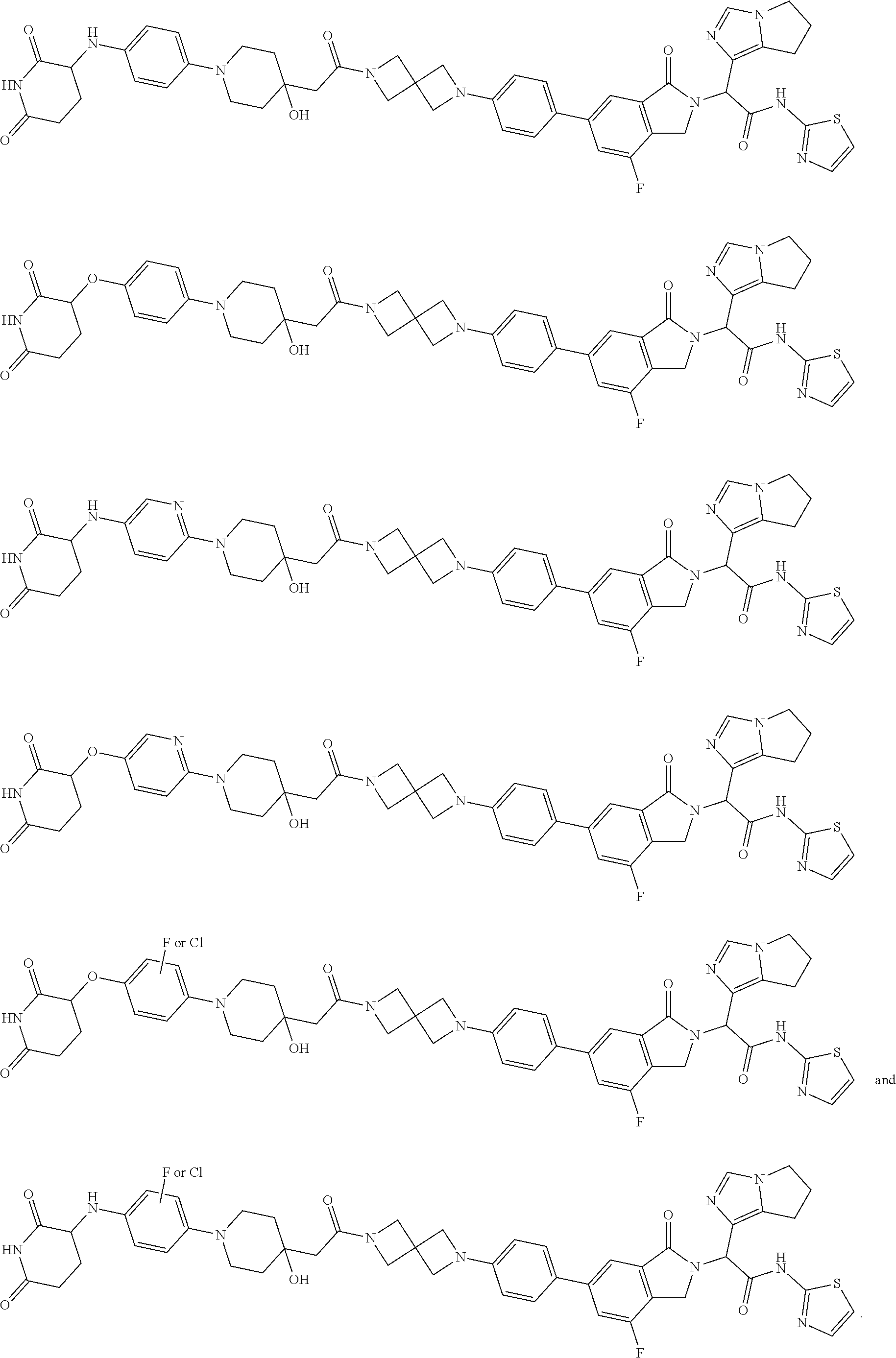

In certain embodiments the compound for use in the methods of treatment described herein is selected from:

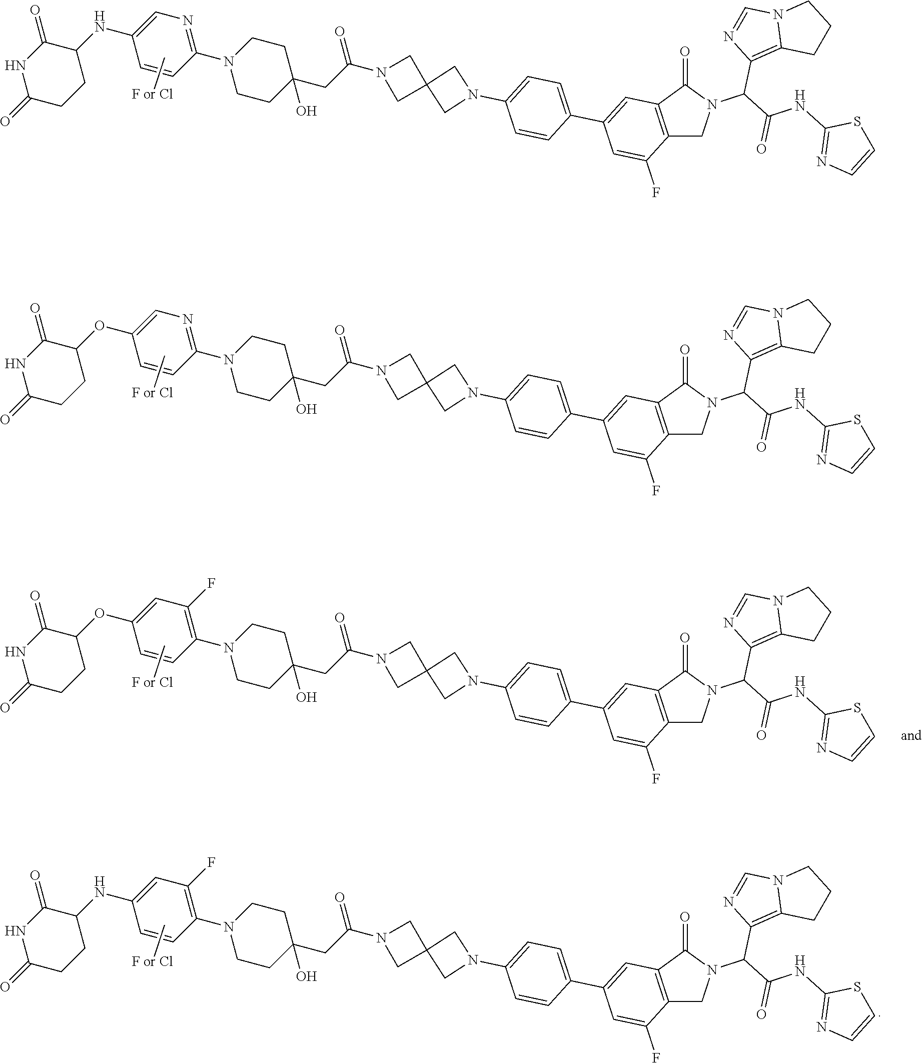

In certain embodiments the compound for use in the methods of treatment described herein is selected from:

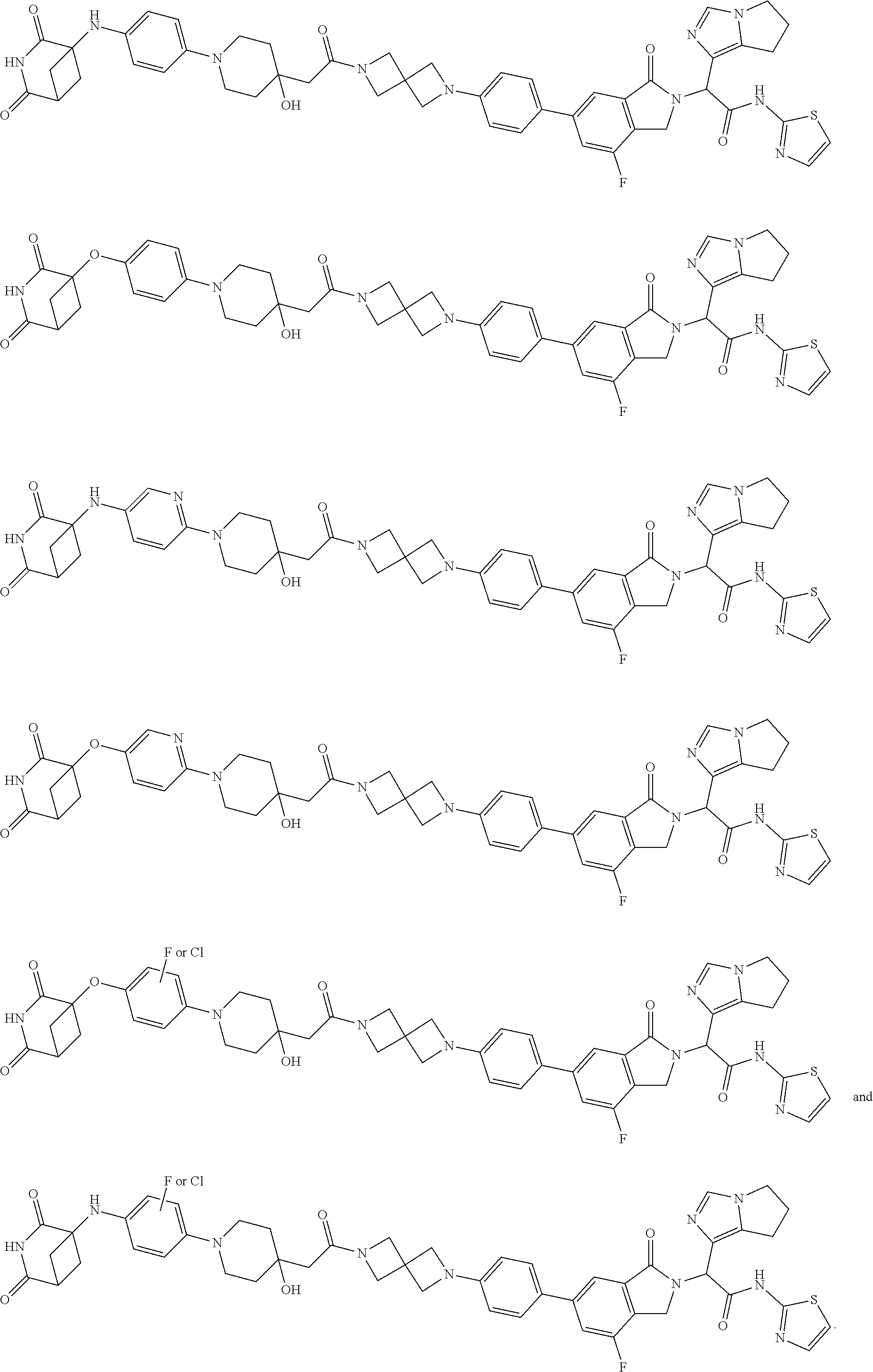

In certain embodiments the compound for use in the methods of treatment described herein is selected from:

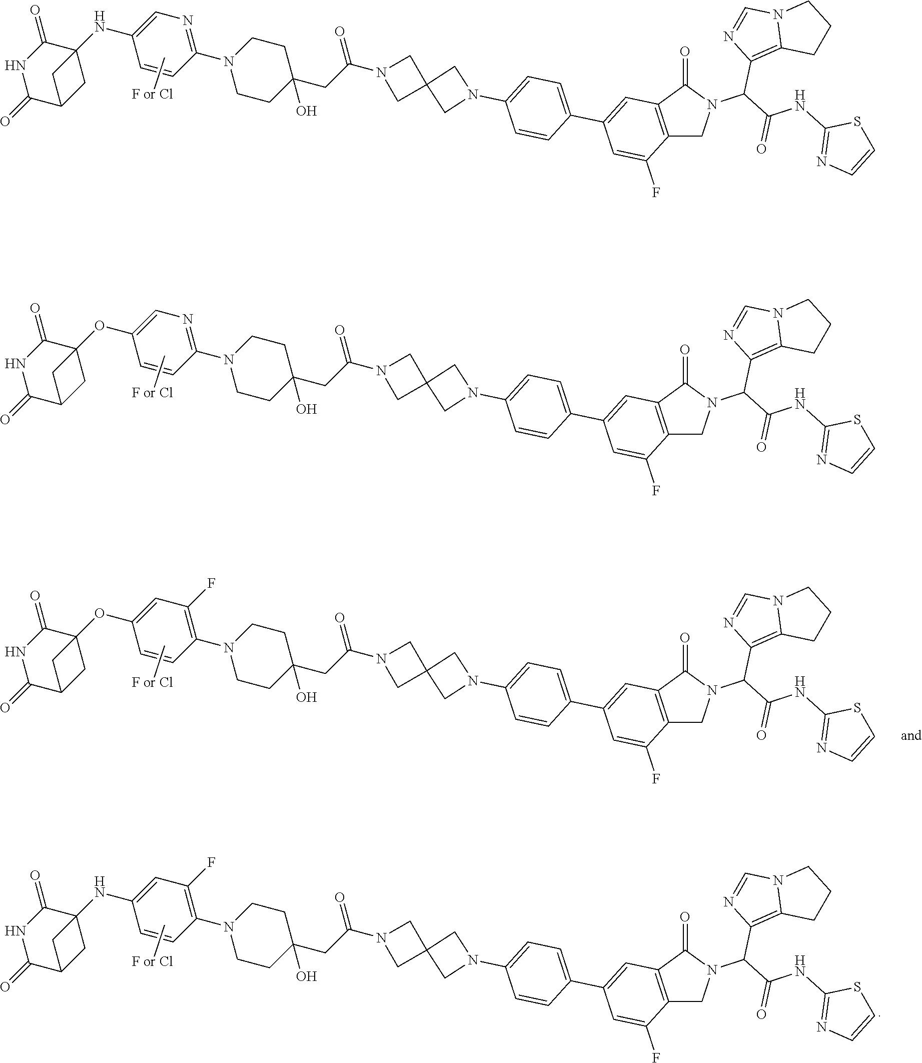

In certain embodiments the compound for use in the methods of treatment described herein is selected from:

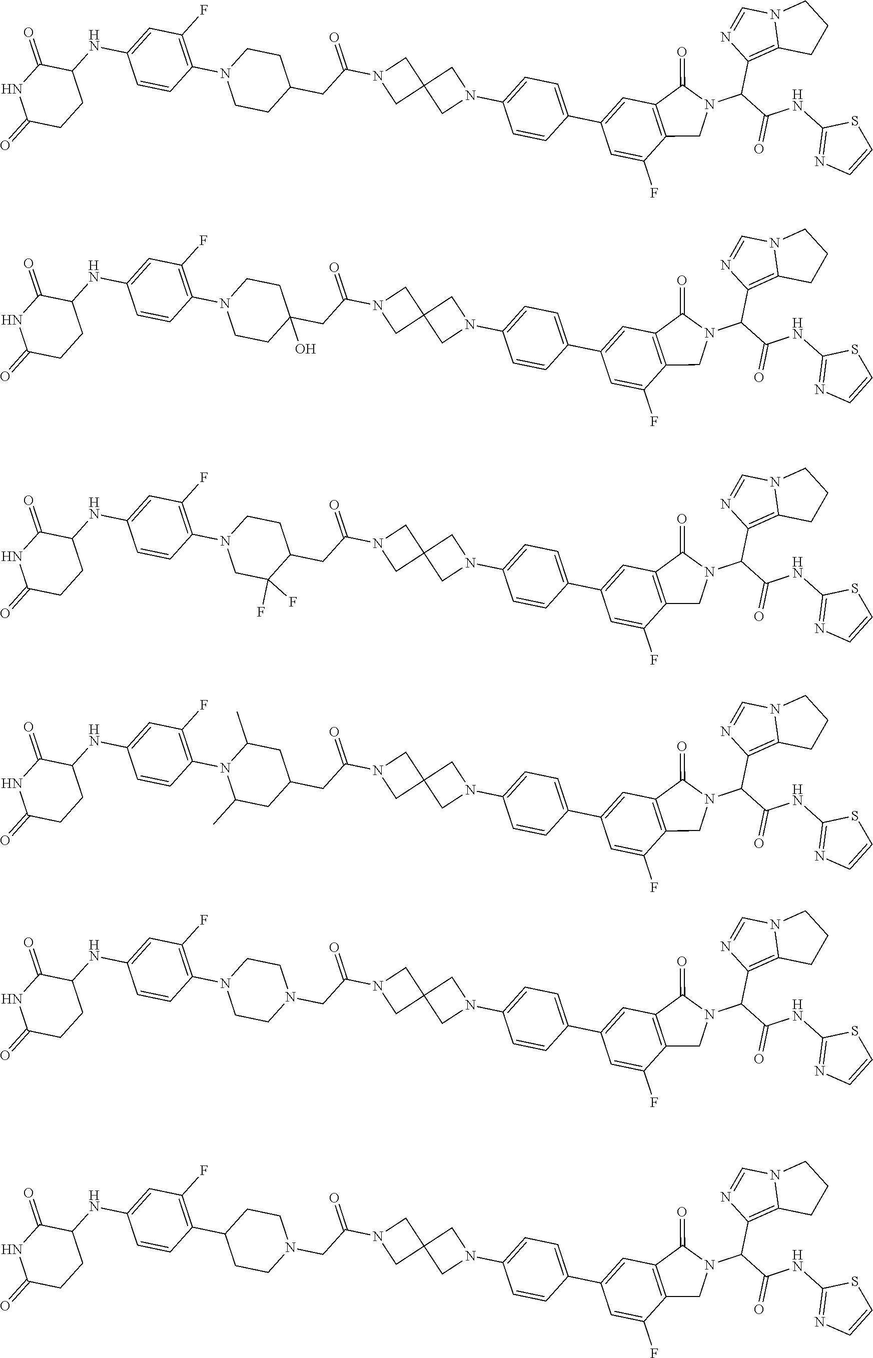

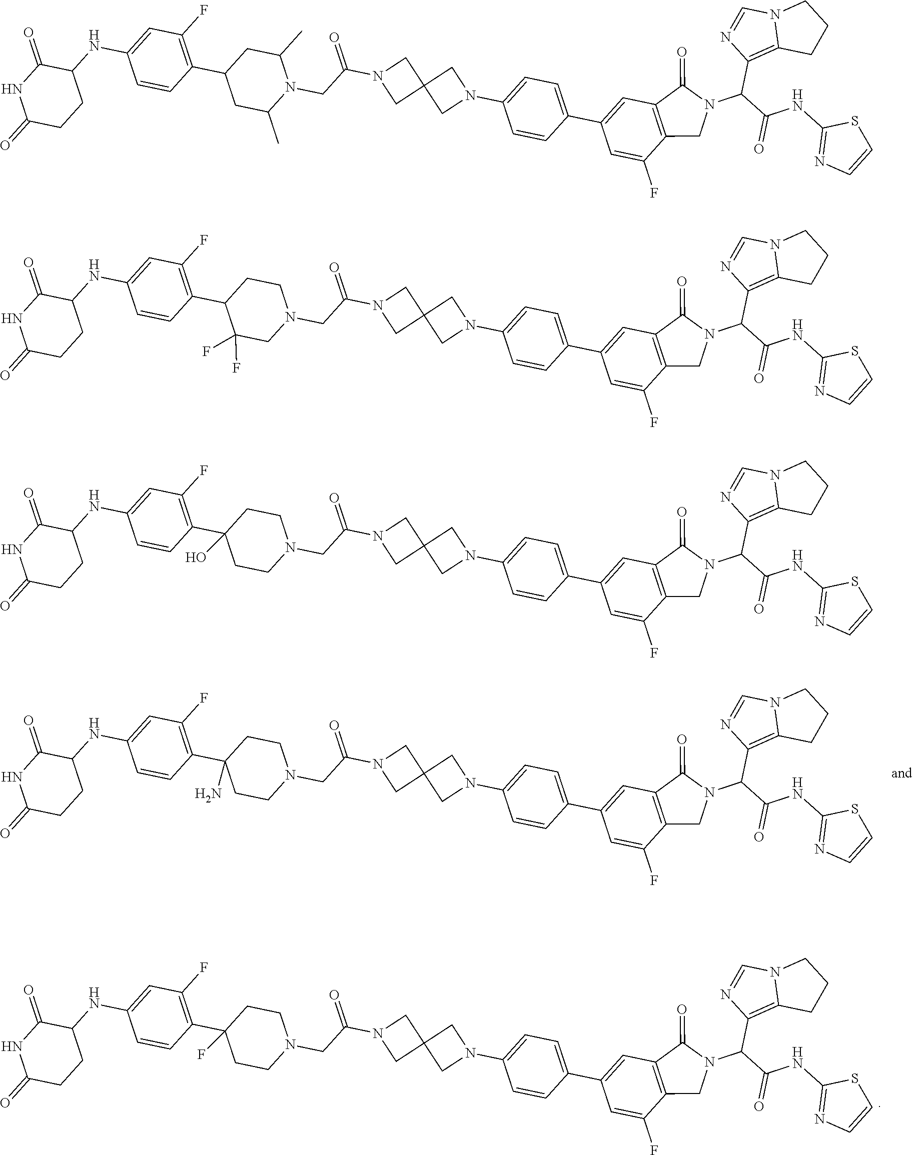

In certain embodiments the compound for use in the methods of treatment described herein is selected from:

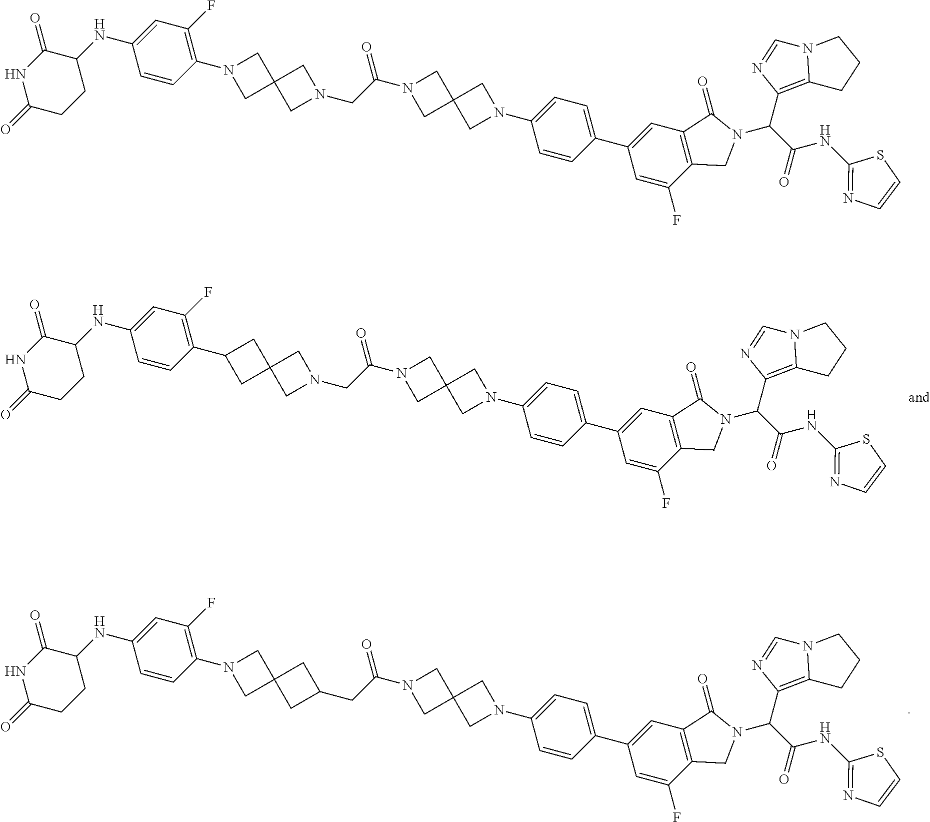 [SMILES: O=C1CCC(Nc2ccc(N3CC4(CN(CC(=O)N5CC6(C5)CN(c5ccc(-c7cc(F)c8c(c7)C(=O)N(C(C(=O)Nc7nccs7)c7ncn9c7CCC9)C8)cc5)C6)C4)C3)c(F)c2)C(=O)N1 ; O=C1CCC(Nc2ccc(C3CC4(C3)CN(CC(=O)N3CC5(C3)CN(c3ccc(-c6cc(F)c7c(c6)C(=O)N(C(C(=O)Nc6nccs6)c6ncn8c6CCC8)C7)cc3)C5)C4)c(F)c2)C(=O)N1 ; O=C1CCC(Nc2ccc(N3CC4(CC(CC(=O)N5CC6(C5)CN(c5ccc(-c7cc(F)c8c(c7)C(=O)N(C(C(=O)Nc7nccs7)c7ncn9c7CCC9)C8)cc5)C6)C4)C3)c(F)c2)C(=O)N1]

In certain embodiments the compound for use in the methods of treatment described herein is selected from:

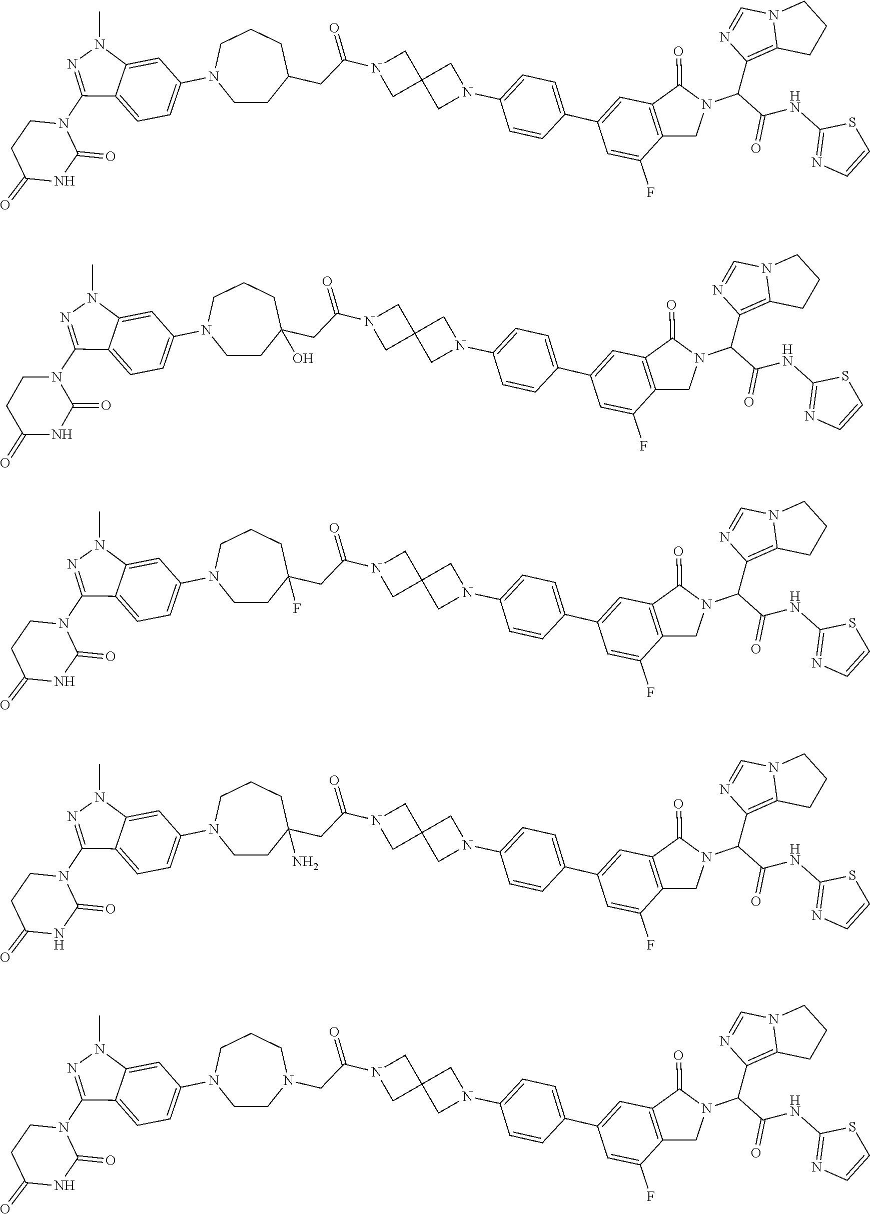

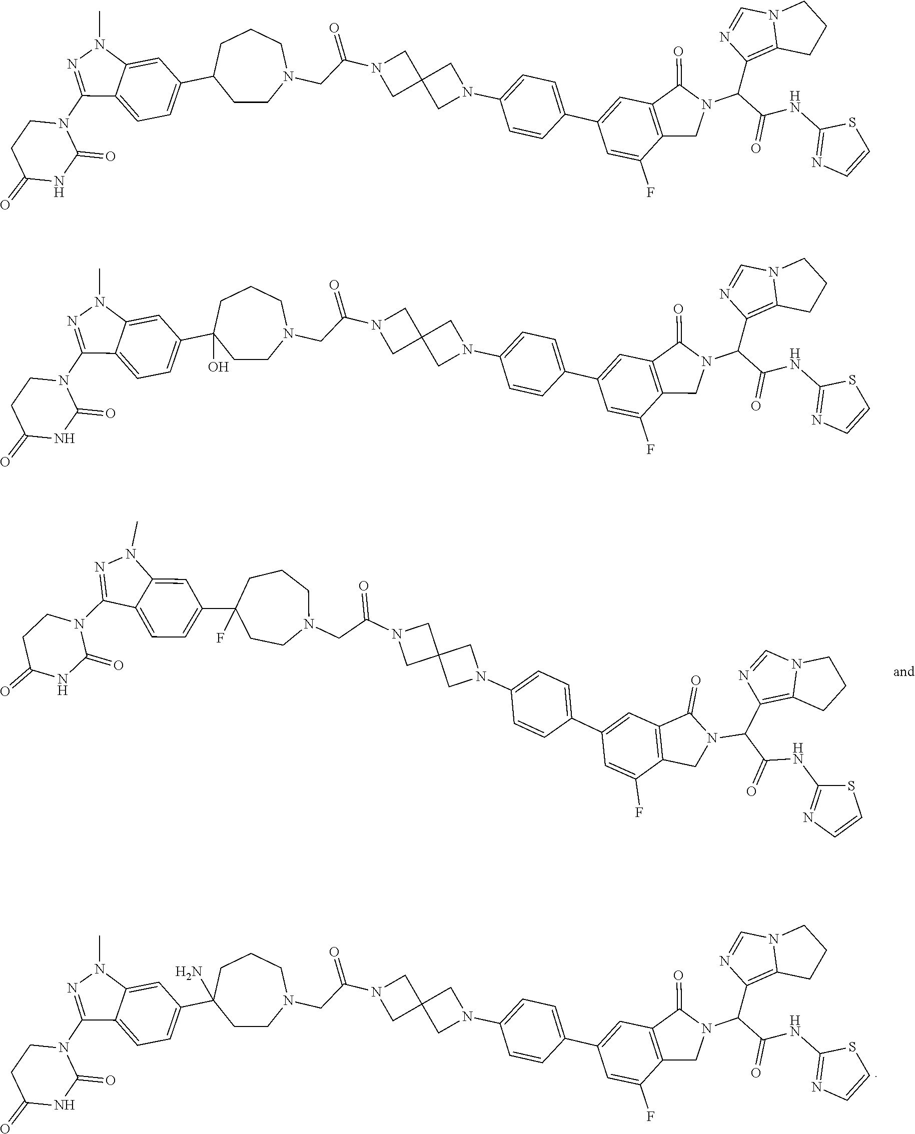

In certain embodiments the compound for use in the methods of treatment described herein is selected from:

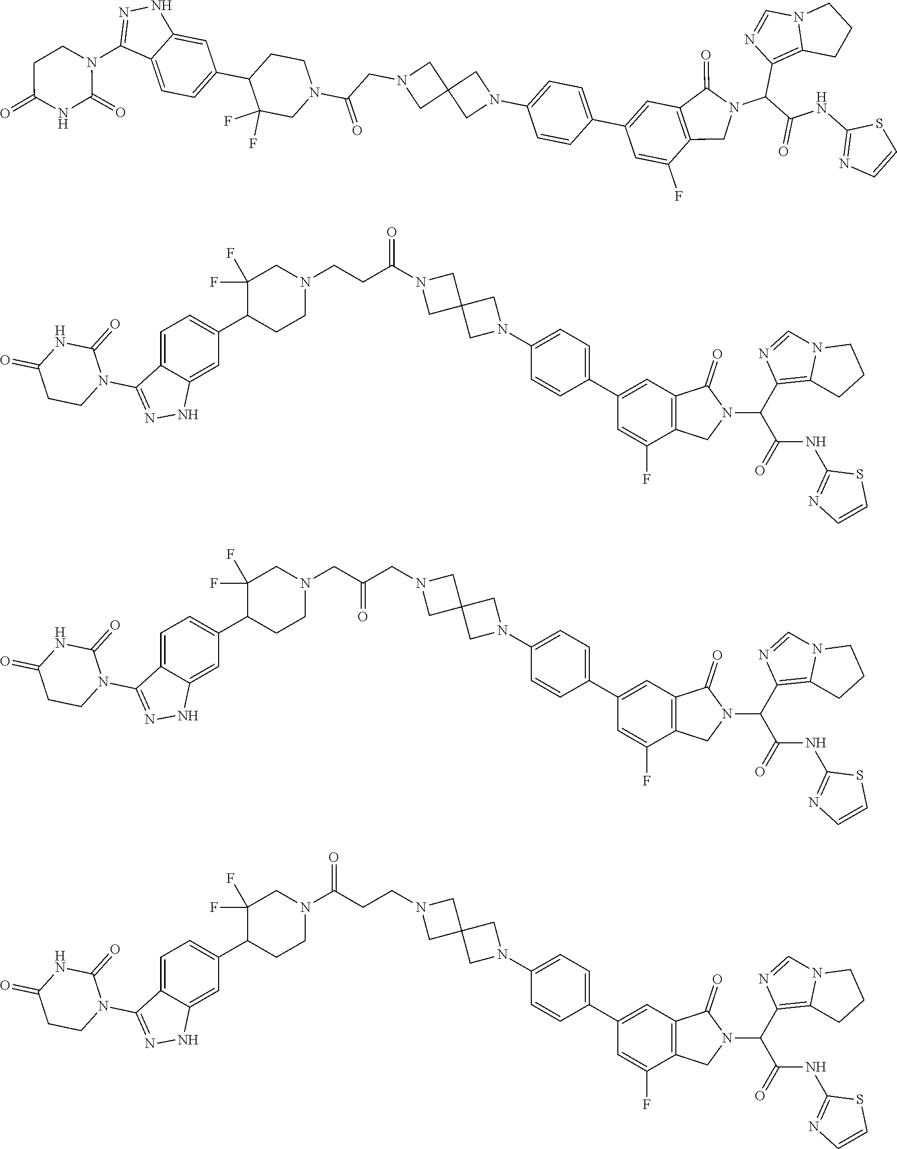

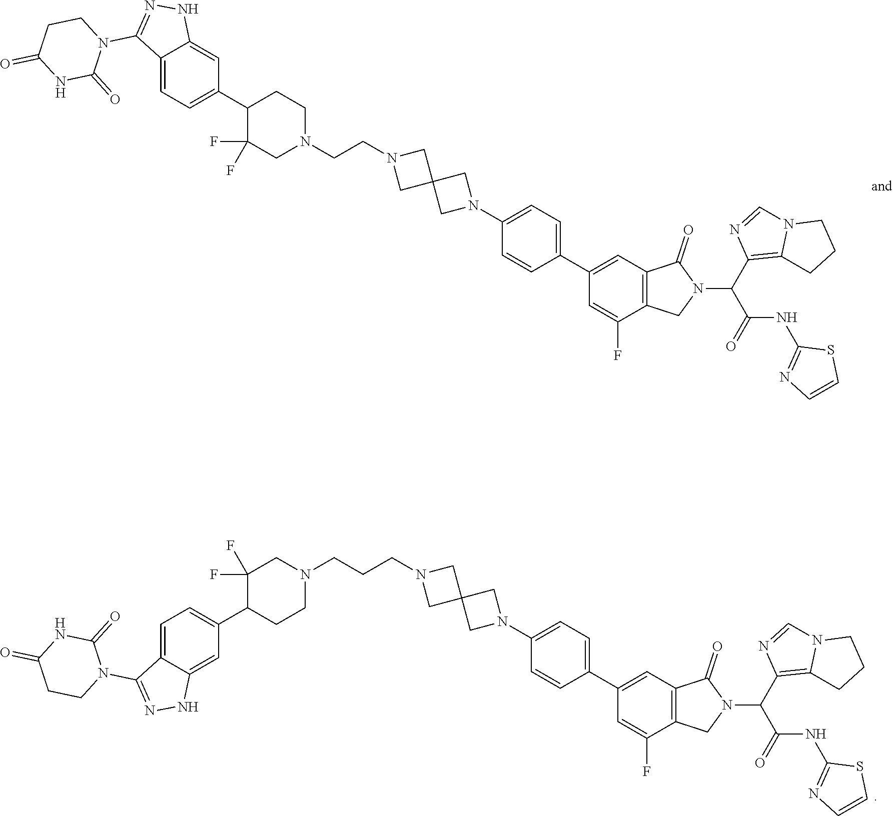 [SMILES: O=C1CCN(c2n[nH]c3cc(C4CCN(CCN5CC6(C5)CN(c5ccc(-c7cc(F)c8c(c7)C(=O)N(C(C(=O)Nc7nccs7)c7ncn9c7CCC9)C8)cc5)C6)CC4(F)F)ccc23)C(=O)N1 ; O=C1CCN(c2n[nH]c3cc(C4CCN(CCCN5CC6(C5)CN(c5ccc(-c7cc(F)c8c(c7)C(=O)N(C(C(=O)Nc7nccs7)c7ncn9c7CCC9)C8)cc5)C6)CC4(F)F)ccc23)C(=O)N1]

In certain embodiments the compound for use in the methods of treatment described herein is selected from:

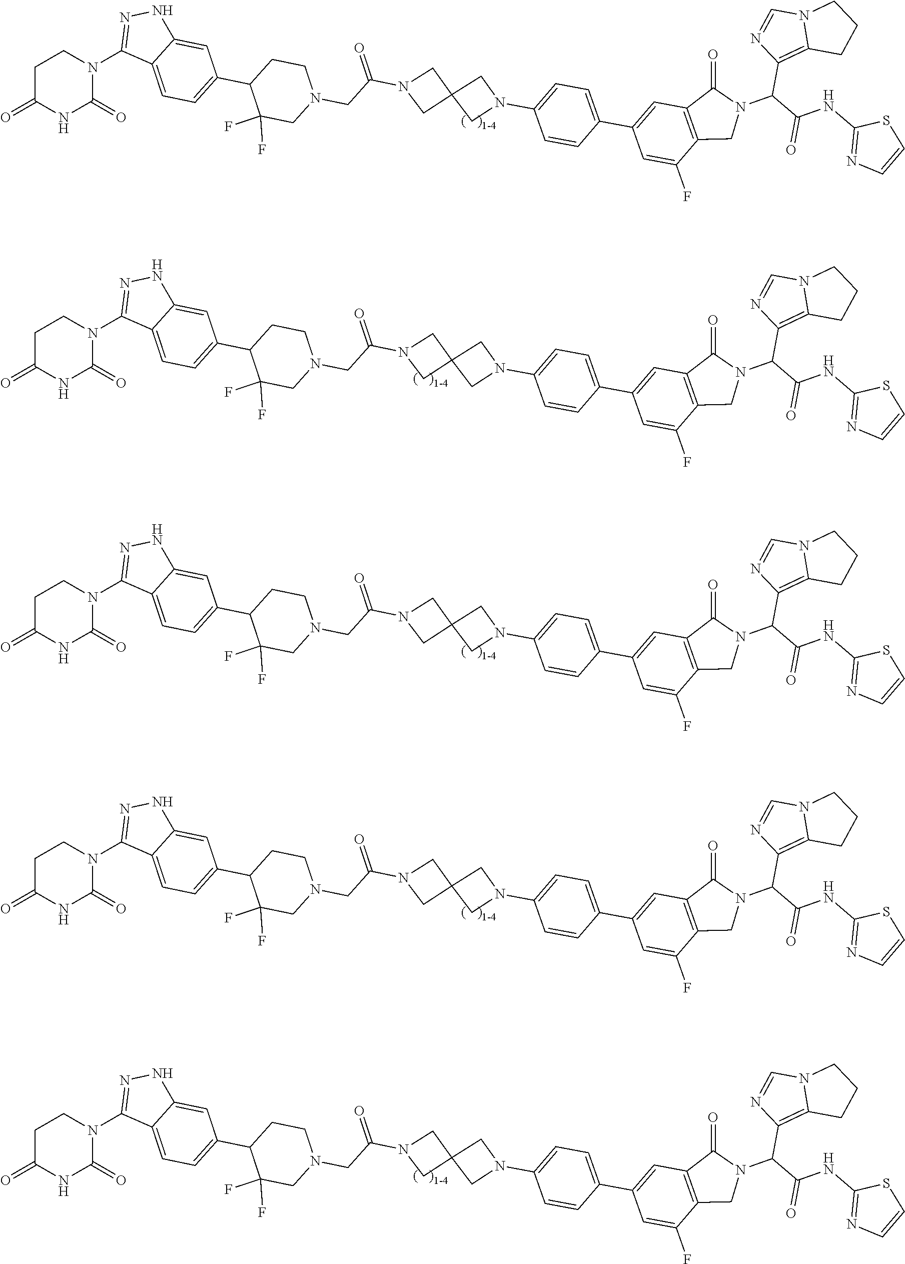

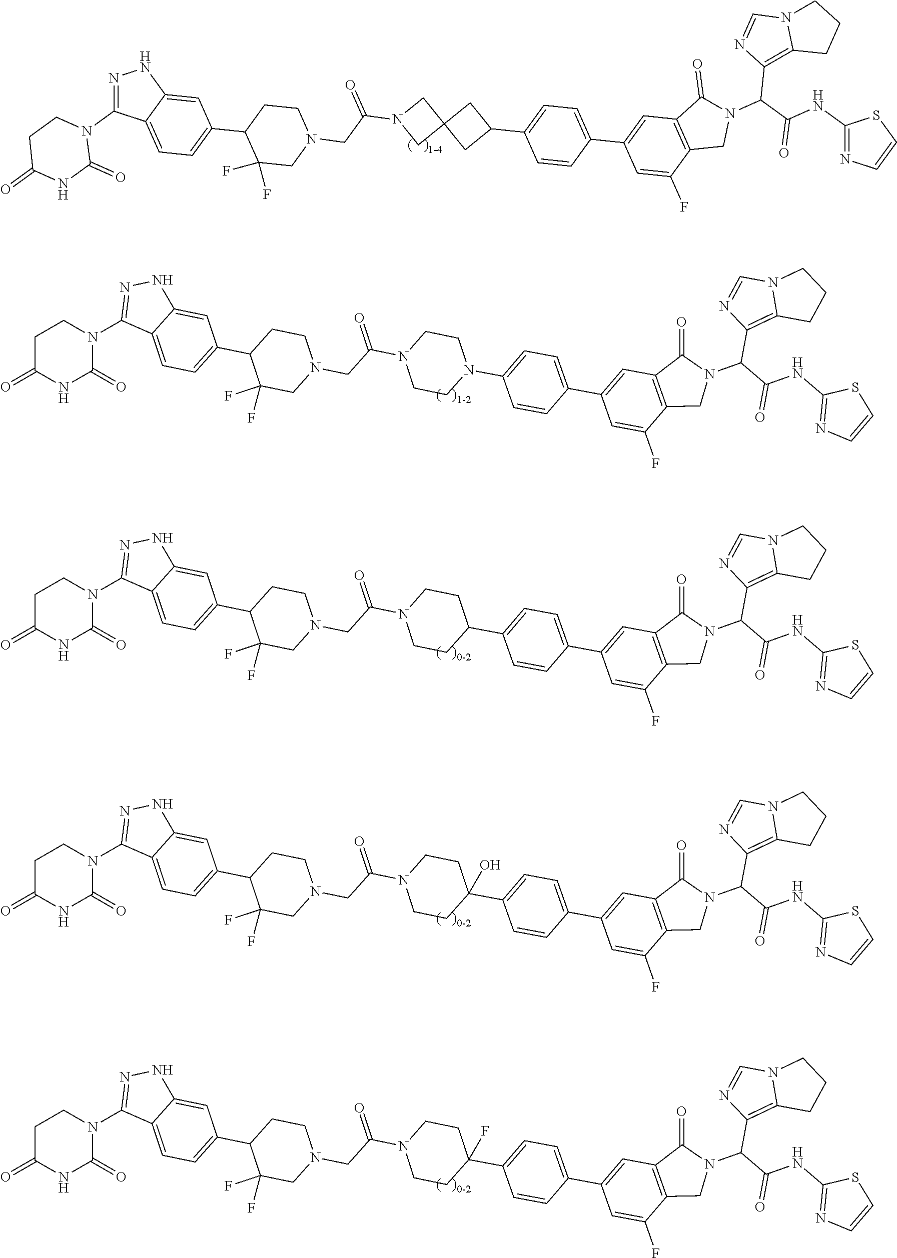

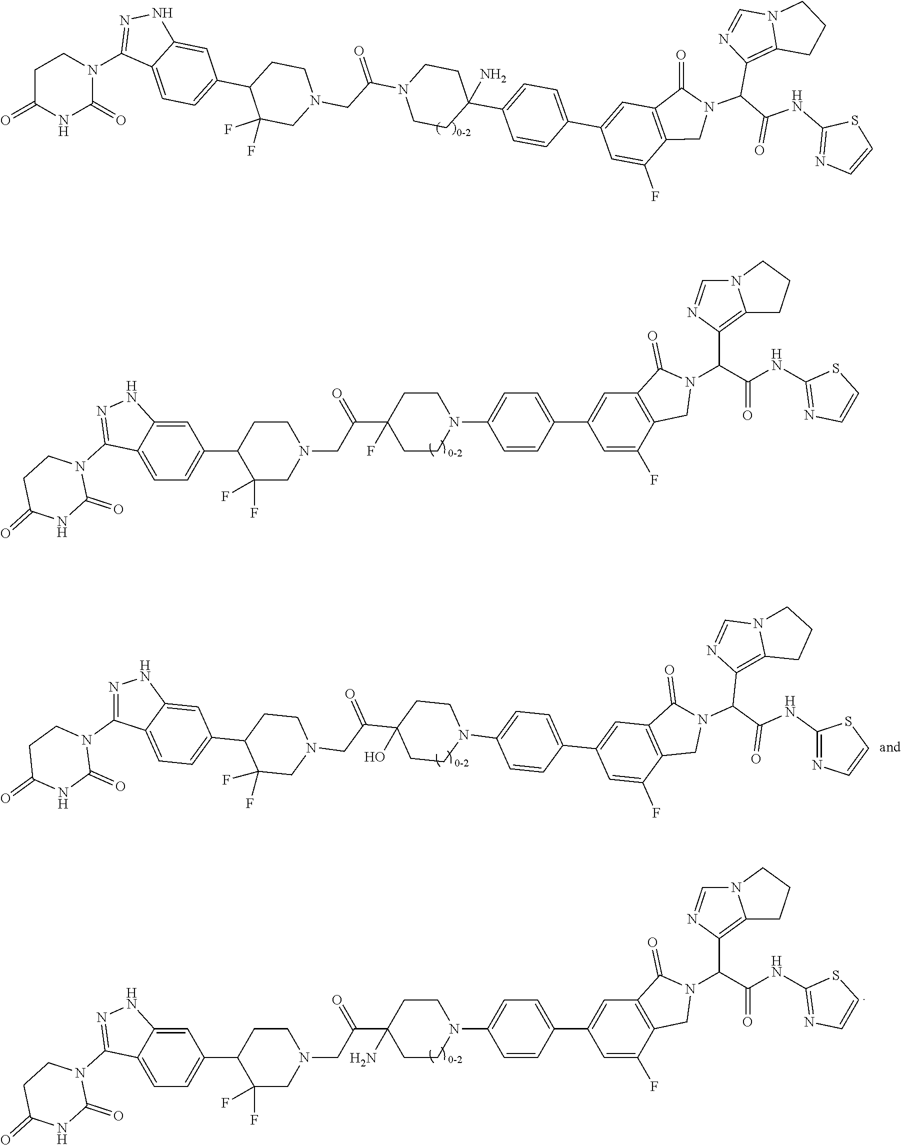

In certain embodiments the compound for use in the methods of treatment described herein is selected from:

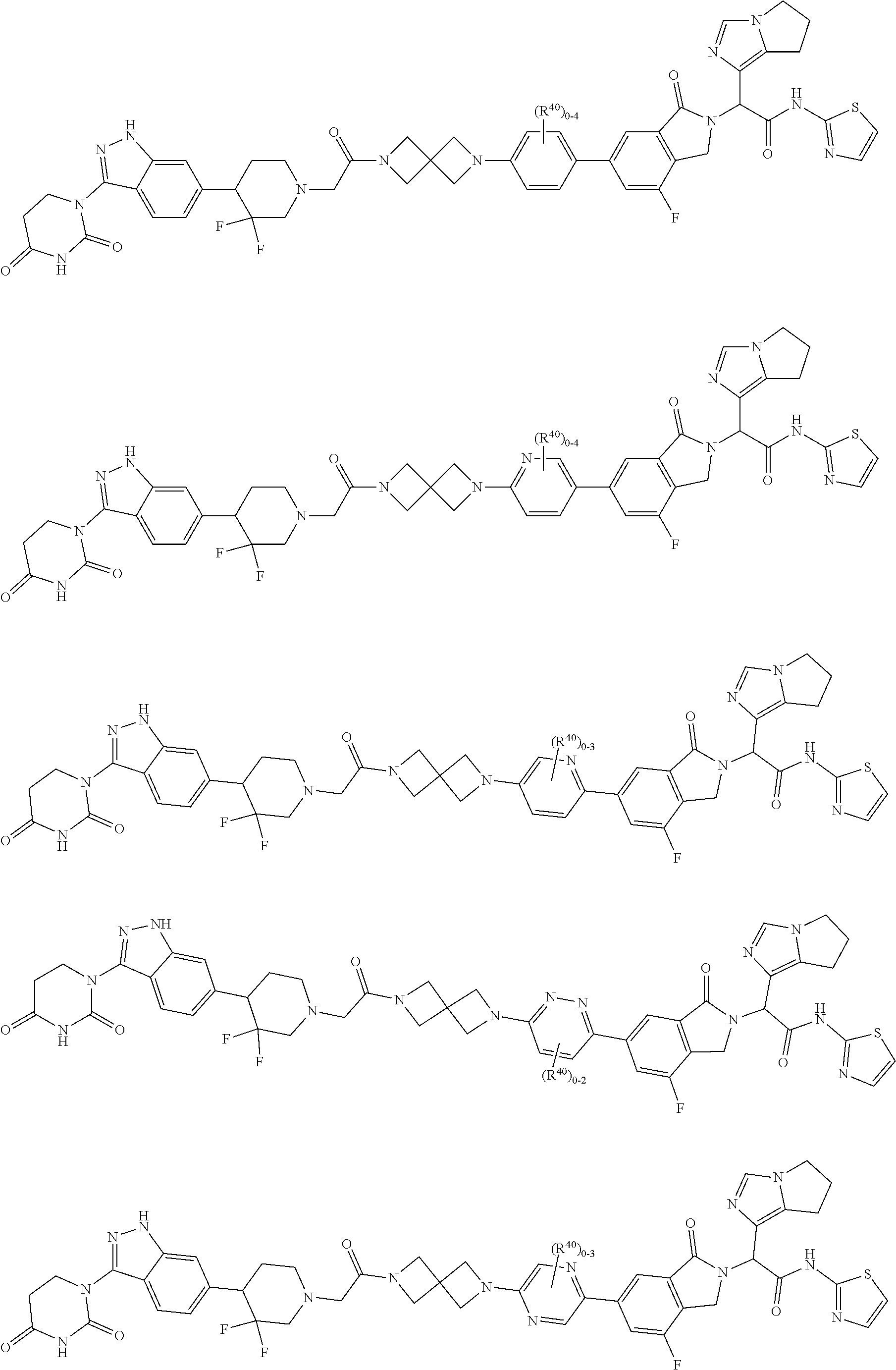

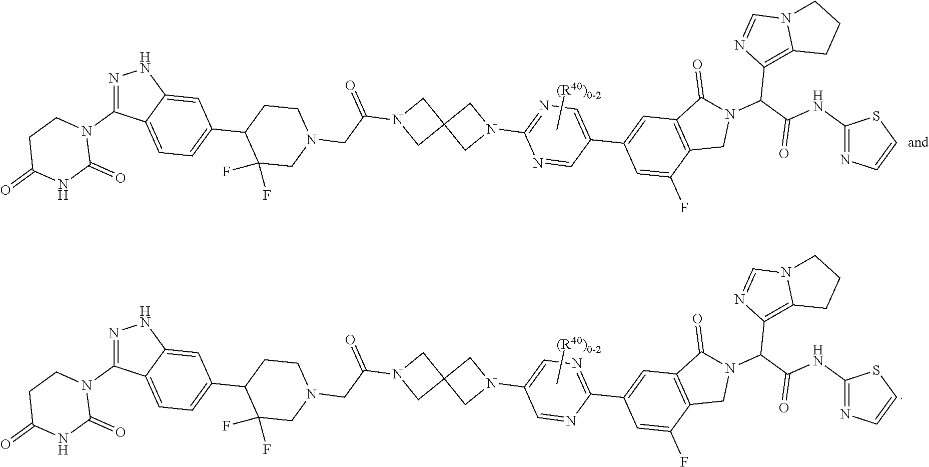

In certain embodiments the compound for use in the methods of treatment described herein is selected from:

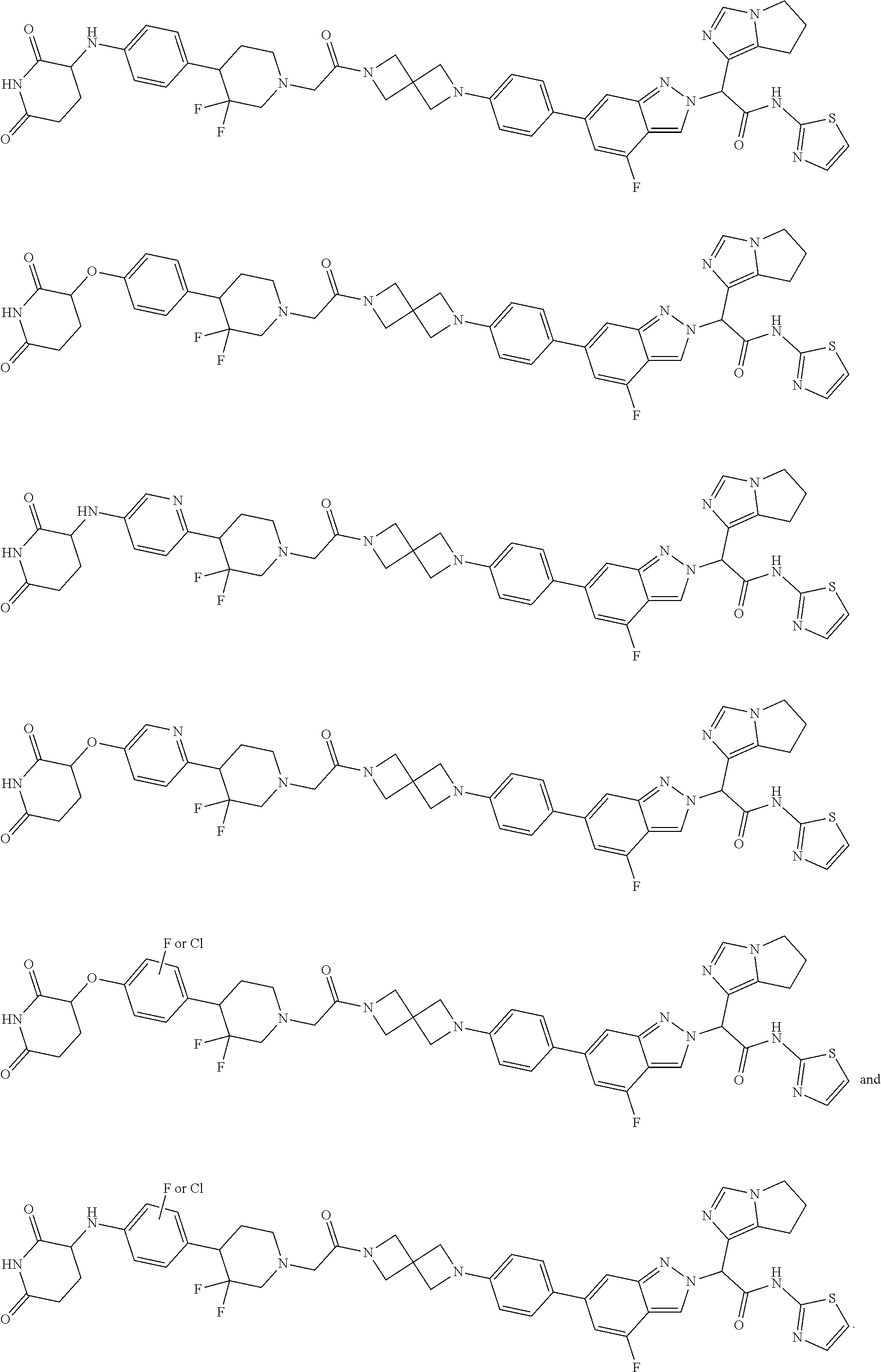

In certain embodiments the compound for use in the methods of treatment described herein is selected from:

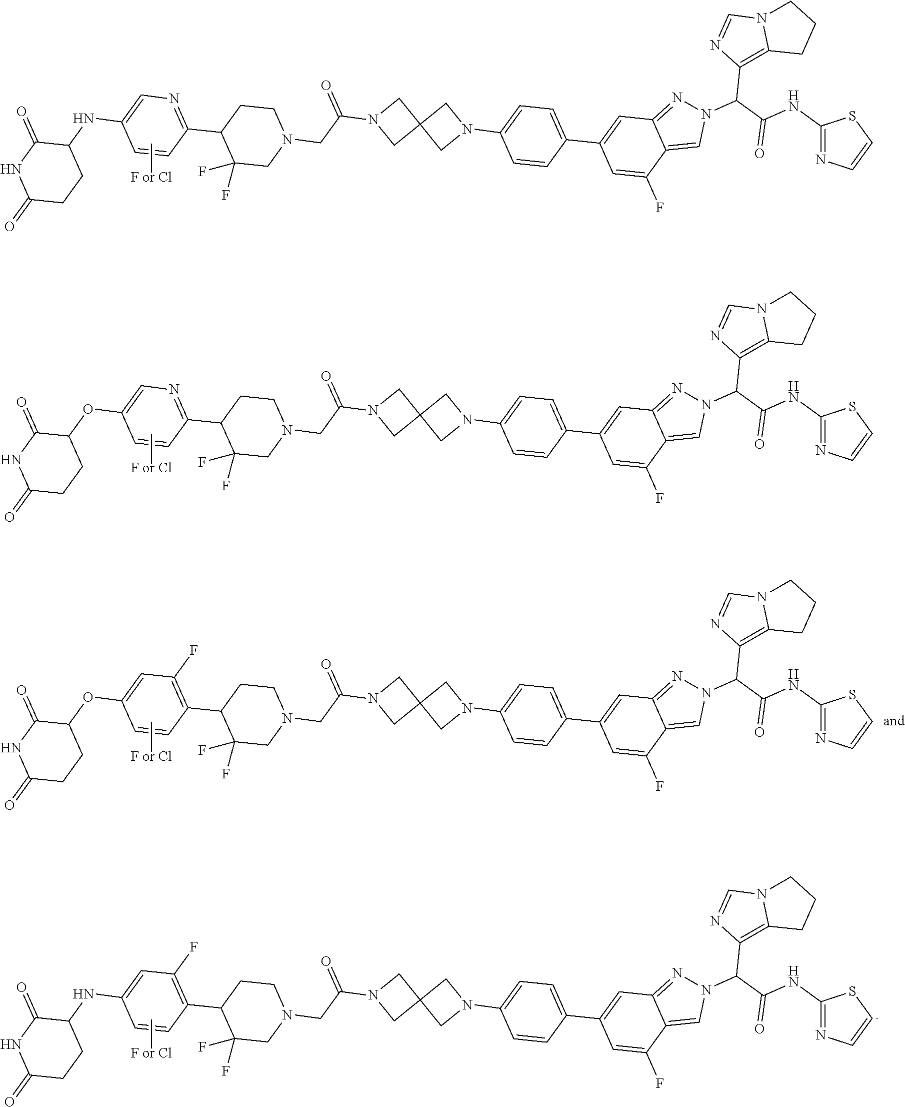

In certain embodiments the compound for use in the methods of treatment described herein is selected from:

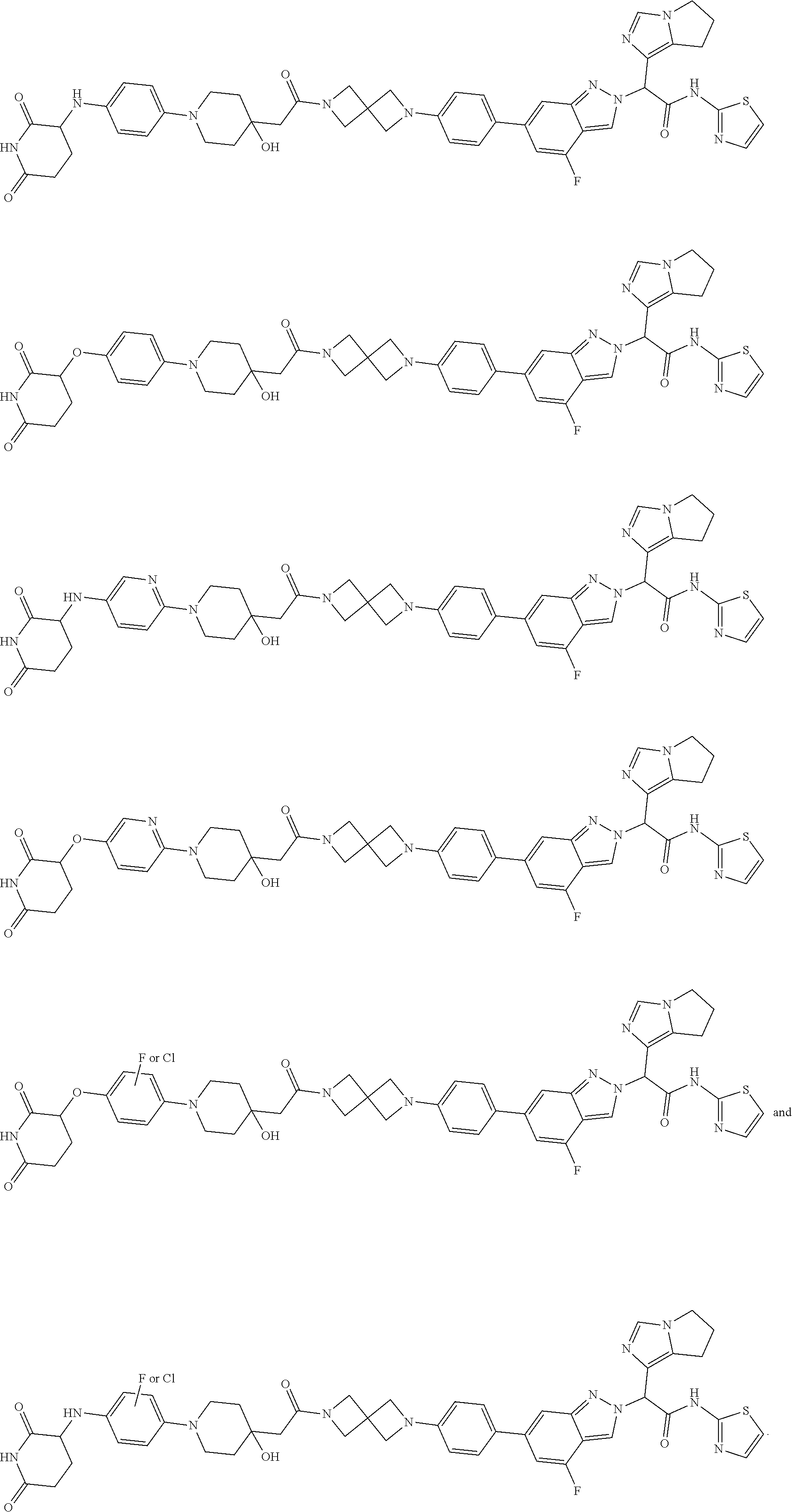

In certain embodiments the compound for use in the methods of treatment described herein is selected from:

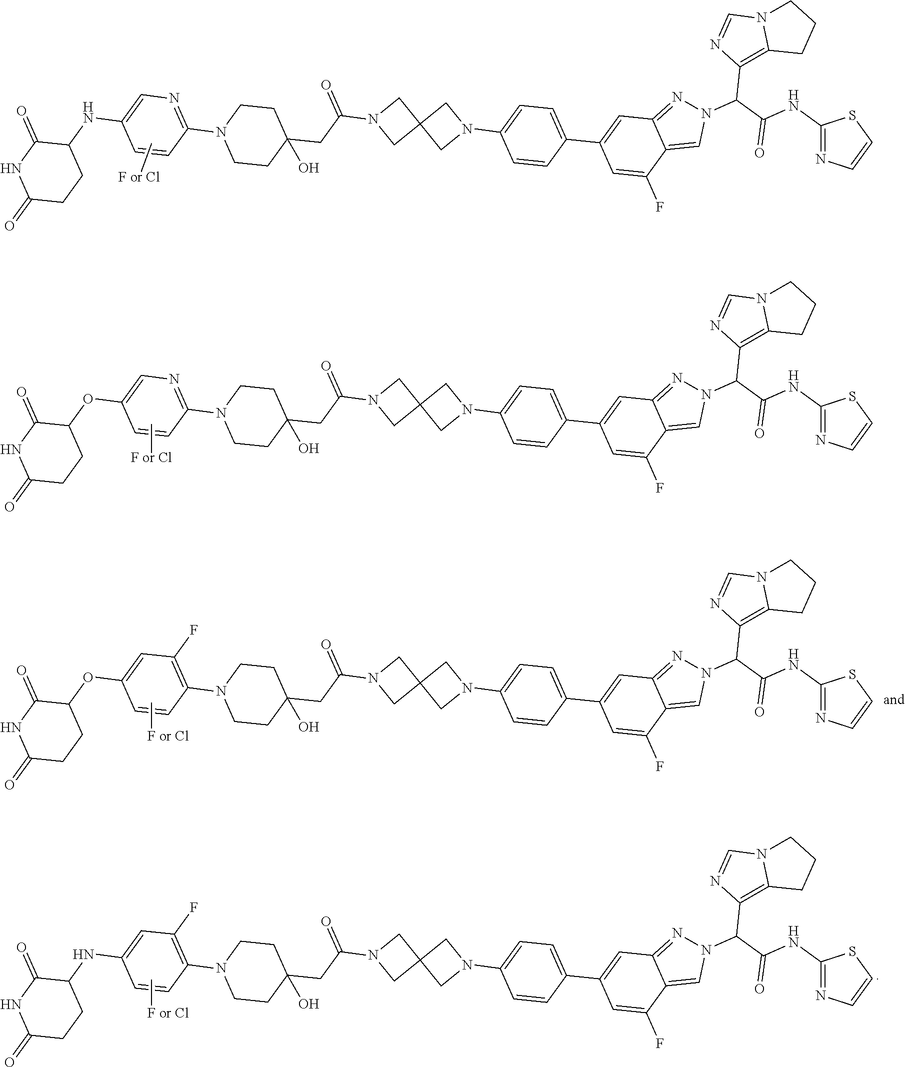

In certain embodiments the compound for use in the methods of treatment described herein is selected from:

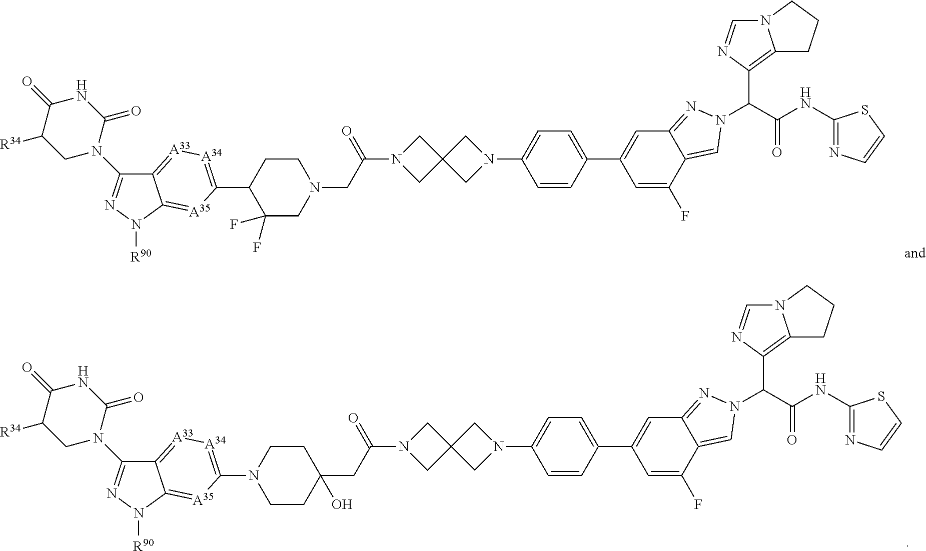

In certain embodiments the compound for use in the methods of treatment described herein is selected from:

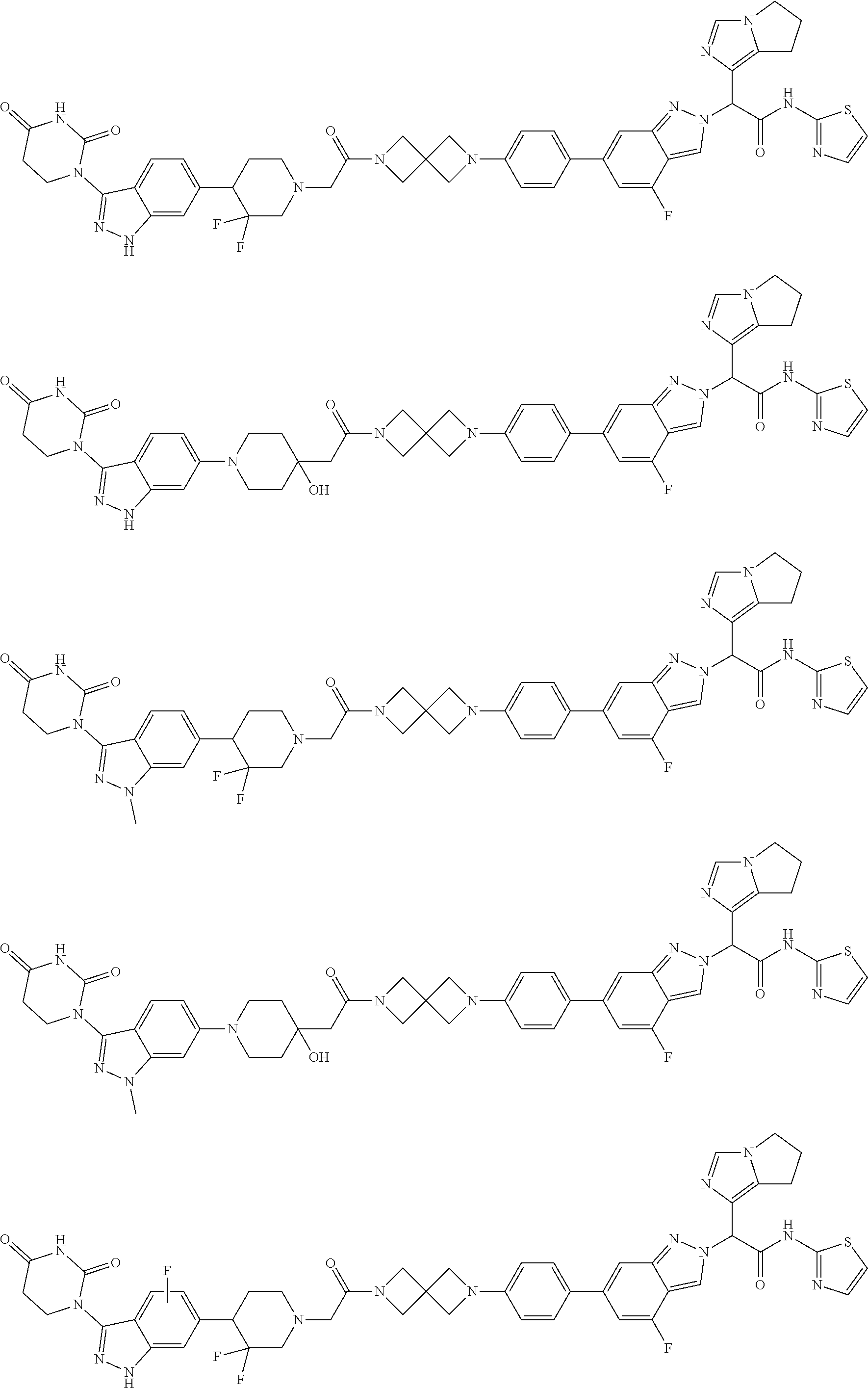

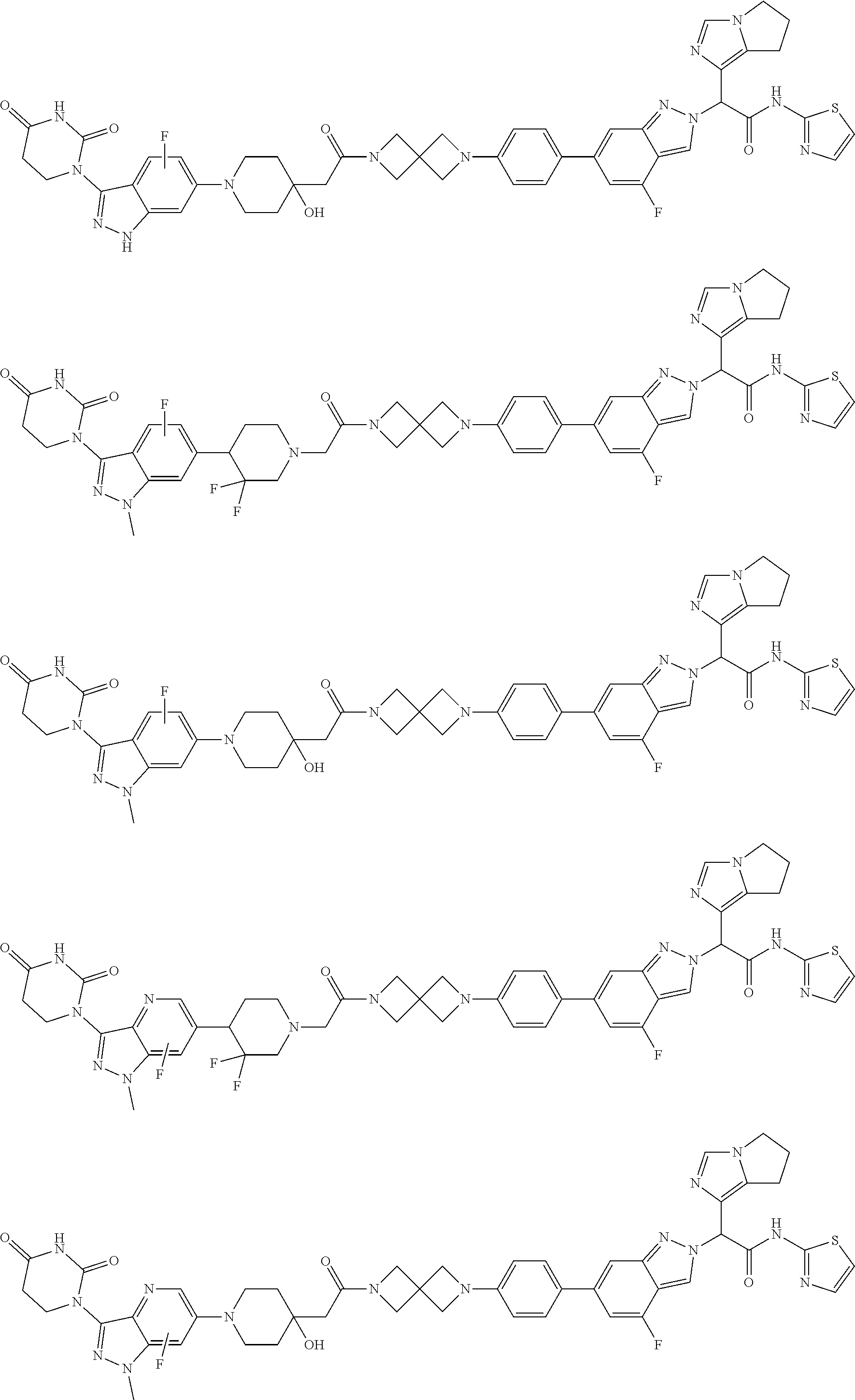

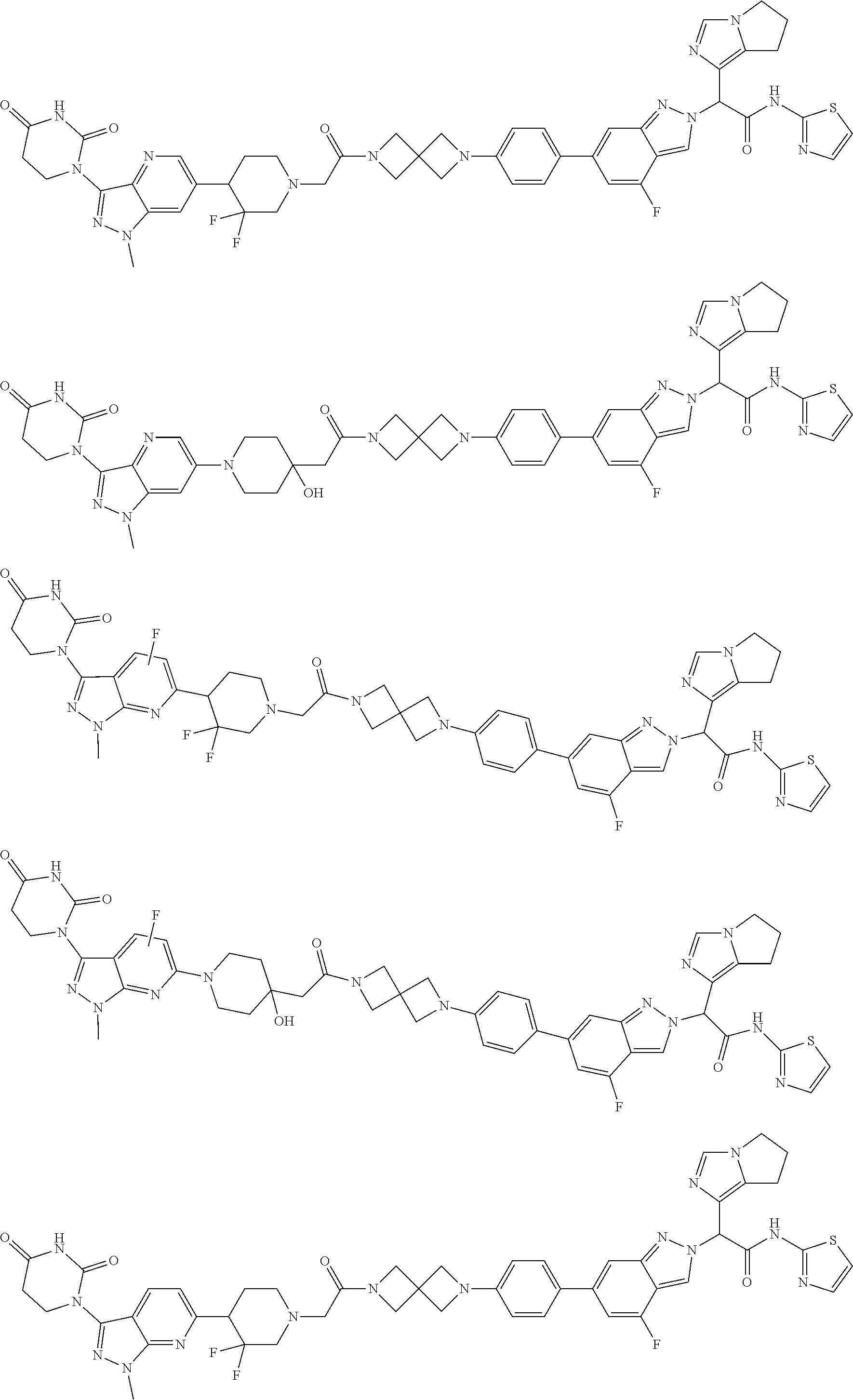

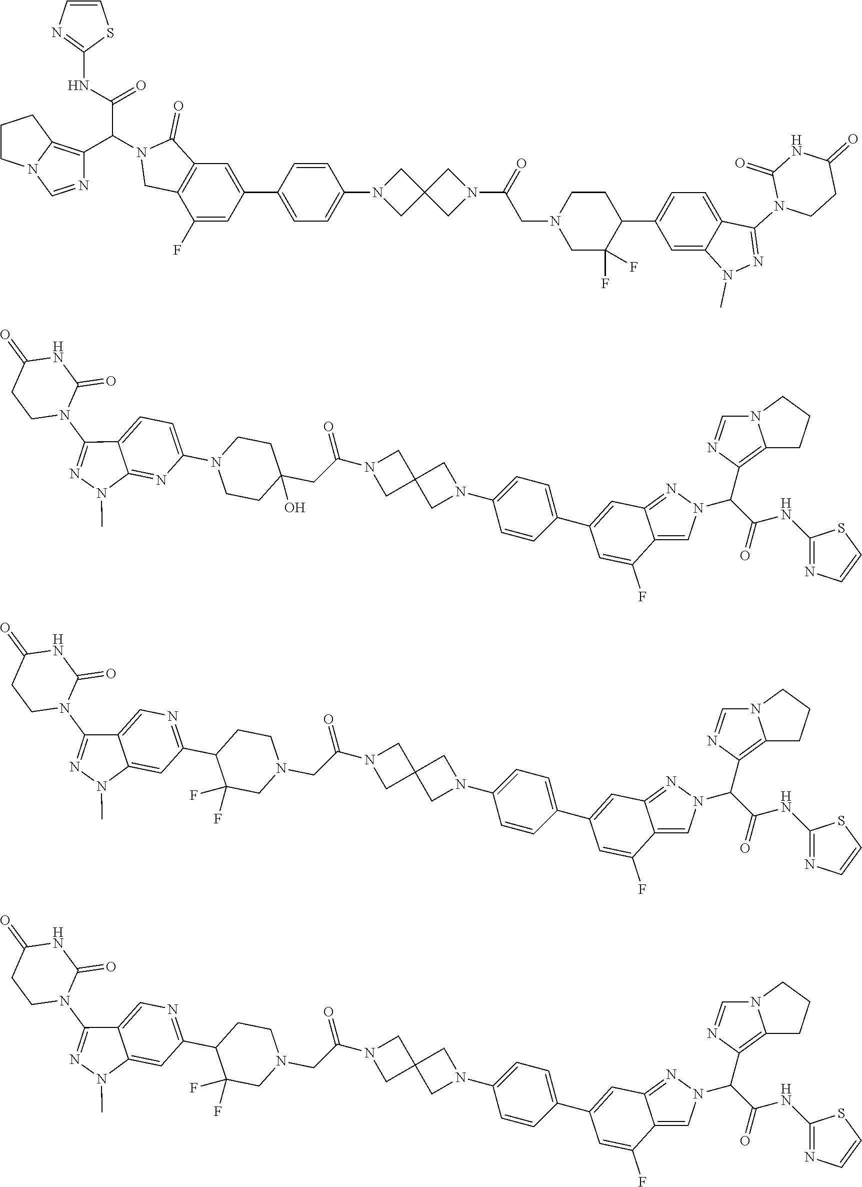

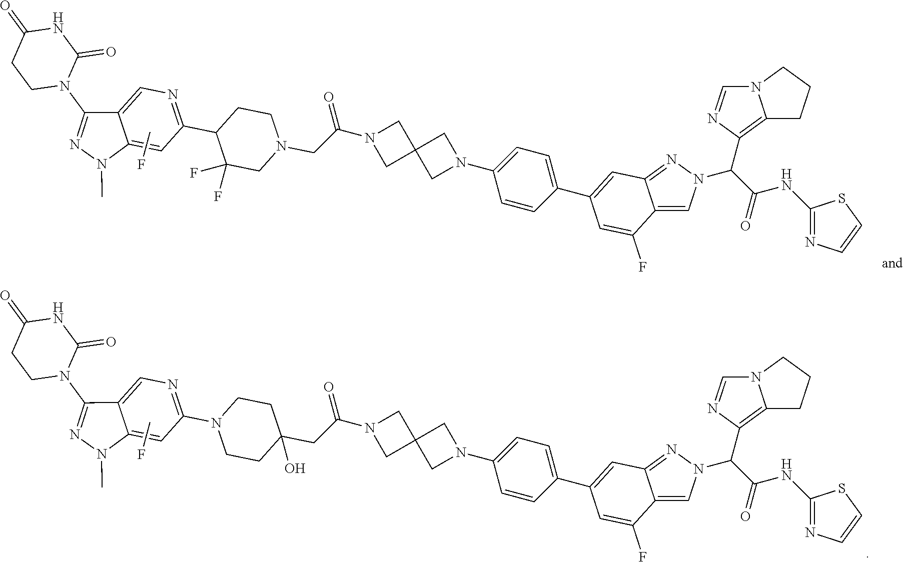

### IV. Linkers

A Linker (L¹ or L²) or a bond is included in the compounds described herein. Linker is a chemically stable bivalent group that attaches an E3 Ligase binding portion to an EGFR Targeting Ligand. According to the invention, any desired linker, as described herein, can be used as long as the resulting compound has a stable shelf life, for example at least 1 month, 2 months, 3 months, 6 months or 1 year as part of a pharmaceutically acceptable dosage form, and itself is pharmaceutically acceptable.

Linker as described herein can be used in either direction, i.e., either the left end is linked to the E3 Ligase binding portion and the right end to the EGFR Targeting Ligand, or the left end is linked to the EGFR Targeting Ligand and the right end is linked to the E3 Ligase binding portion.

In certain embodiments Linker is a bond.

In certain embodiments, the Linker has a chain of 2 to 14, 15, 16, 17, 18 or 20 or more carbon atoms of which one or more carbons can be replaced by a heteroatom such as O, N, S, or P.

In certain embodiments the chain has 2, 3, 4, 5, 6, 7, 8, 9, 10, 11, 12, 13, 14, 15, 16, 17, 18, 19 or 20 contiguous atoms in the chain. For example, the chain may include 1 or more ethylene glycol units that can be contiguous, partially contiguous or non-contiguous (for example, 2, 3, 4, 5, 6, 7, 8, 9, 10, 11 or 12 ethylene glycol units).

In certain embodiments the chain has at least 1, 2, 3, 4, 5, 6, 7, or 8 contiguous chains which can have branches which can be independently alkyl, aryl, heteroaryl, alkenyl, or alkynyl, aliphatic, heteroaliphatic, cycloalkyl or heterocycle substituents.

In other embodiments, the linker can include or be comprised of one or more of ethylene glycol, propylene glycol, lactic acid and/or glycolic acid. Lactic acid segments tend to have a longer half-life than glycolic acid segments. Block and random lactic acid-co-glycolic acid moieties, as well as ethylene glycol and propylene glycol, are known in the art to be pharmaceutically acceptable and can be modified or arranged to obtain the desired half-life and hydrophilicity. In certain aspects, these units can be flanked or interspersed with other moieties, such as aliphatic, including alkyl, heteroaliphatic, aryl, heteroaryl, heterocycle, cycloalkyl, etc., as desired to achieve the appropriate drug properties.

In certain embodiments, L² is a linker selected from:

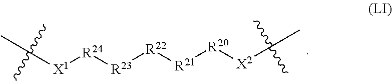

In one aspect, Linker (L²) is selected from the group consisting of a moiety of Formula LI, Formula LII, Formula LIII, Formula LIV, Formula LV, Formula LVI, Formula LVII Formula LVIII, Formula IX and Formula LX:

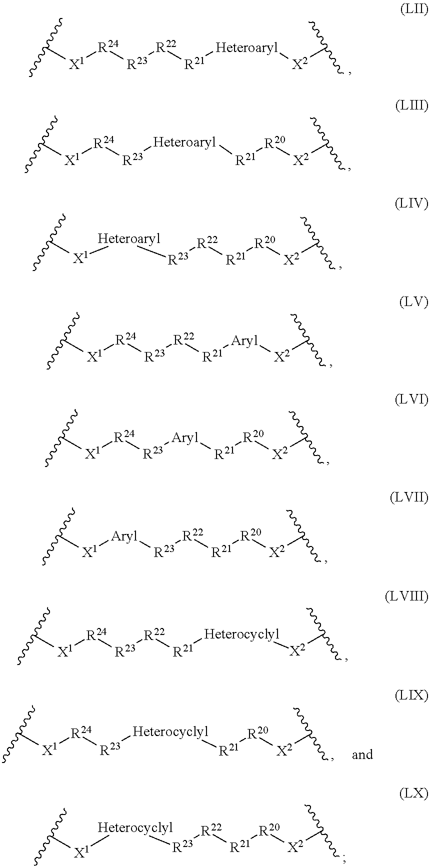

wherein all variables are as defined herein.

In certain embodiments, Linker (L²) is selected from:

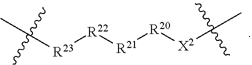

In one aspect, Linker (L²) is selected from the group consisting of a moiety of Formula LDI, Formula LDII, Formula LDIII, Formula LDIV, Formula LDV, Formula LDVI, and Formula LDVII:

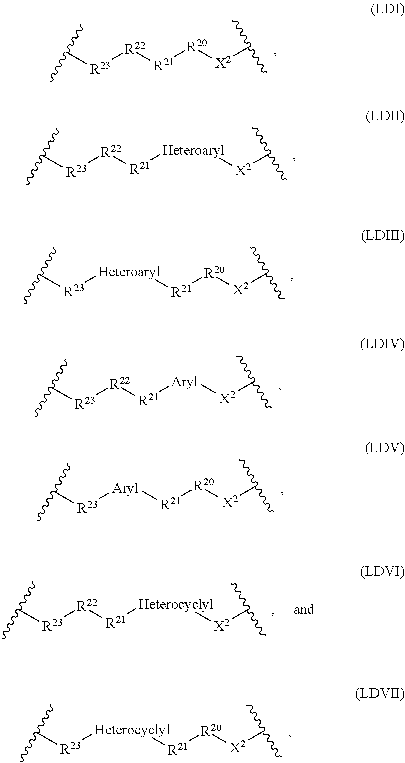

wherein all variables are described herein.

The following are non-limiting examples of Linkers that can be used in this invention. Based on this elaboration, those of skill in the art will understand how to use the full breadth of Linkers that will accomplish the goal of the invention.

In certain embodiments L² is selected from:

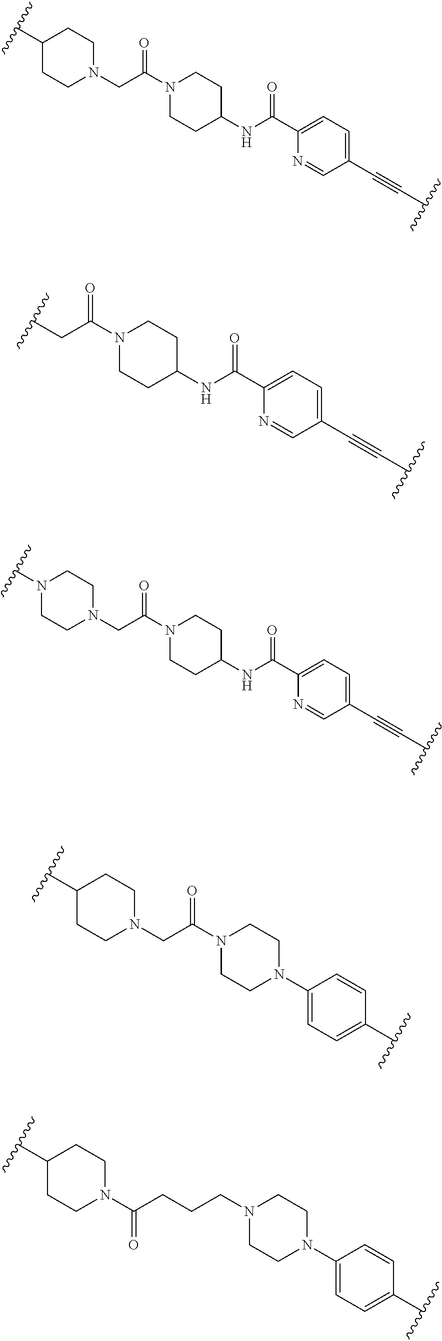 [SMILES: *C#Cc1ccc(C(=O)NC2CCN(C(=O)CN3CCC(*)CC3)CC2)nc1 ; *C#Cc1ccc(C(=O)NC2CCN(C(=O)C*)CC2)nc1 ; *C#Cc1ccc(C(=O)NC2CCN(C(=O)CN3CCN(*)CC3)CC2)nc1 ; *c1ccc(N2CCN(C(=O)CN3CCC(*)CC3)CC2)cc1 ; *c1ccc(N2CCN(CCCC(=O)N3CCC(*)CC3)CC2)cc1]

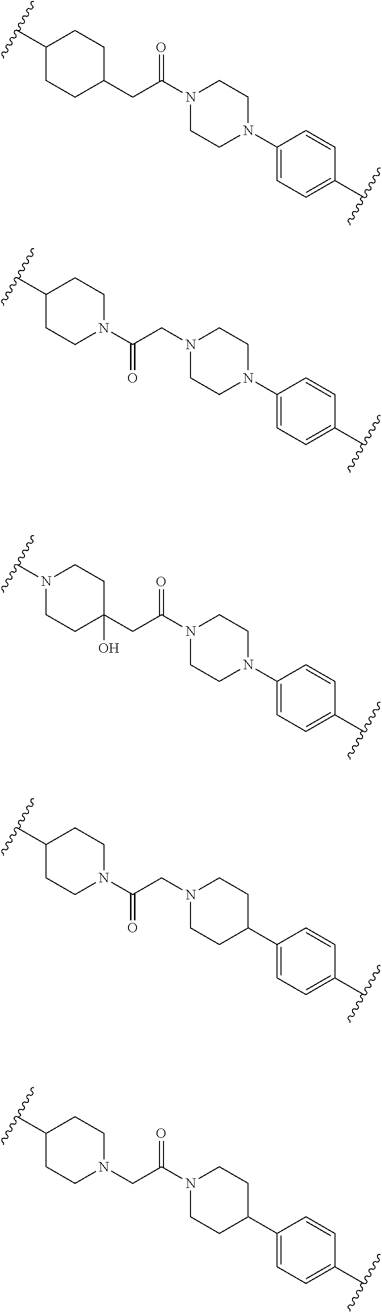 [SMILES: *c1ccc(N2CCN(C(=O)CC3CCC(*)CC3)CC2)cc1 ; *c1ccc(N2CCN(CC(=O)N3CCC(*)CC3)CC2)cc1 ; *c1ccc(N2CCN(C(=O)CC3(O)CCN(*)CC3)CC2)cc1 ; *c1ccc(C2CCN(CC(=O)N3CCC(*)CC3)CC2)cc1 ; *c1ccc(C2CCN(C(=O)CN3CCC(*)CC3)CC2)cc1]

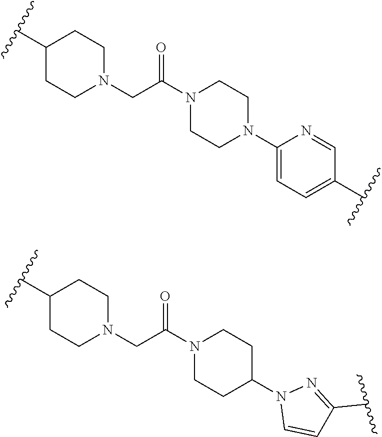 [SMILES: *c1ccc(N2CCN(C(=O)CN3CCC(*)CC3)CC2)nc1 ; *c1ccn(C2CCN(C(=O)CN3CCC(*)CC3)CC2)n1]

In certain embodiments L² is selected from:

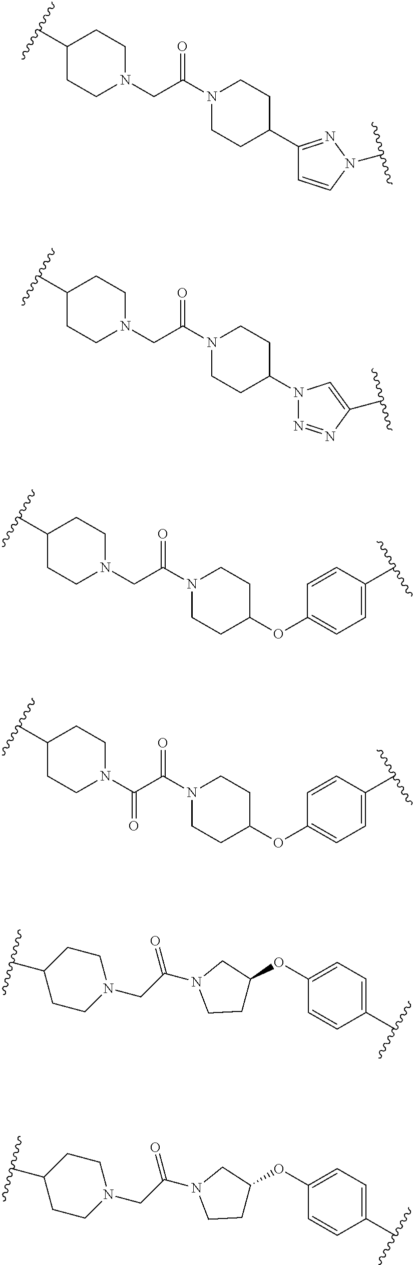 [SMILES: *C1CCN(CC(=O)N2CCC(c3ccn(*)n3)CC2)CC1 ; *c1cn(C2CCN(C(=O)CN3CCC(*)CC3)CC2)nn1 ; *c1ccc(OC2CCN(C(=O)CN3CCC(*)CC3)CC2)cc1 ; *c1ccc(OC2CCN(C(=O)C(=O)N3CCC(*)CC3)CC2)cc1 ; *c1ccc(O[C@H]2CCN(C(=O)CN3CCC(*)CC3)C2)cc1 ; *c1ccc(O[C@@H]2CCN(C(=O)CN3CCC(*)CC3)C2)cc1]

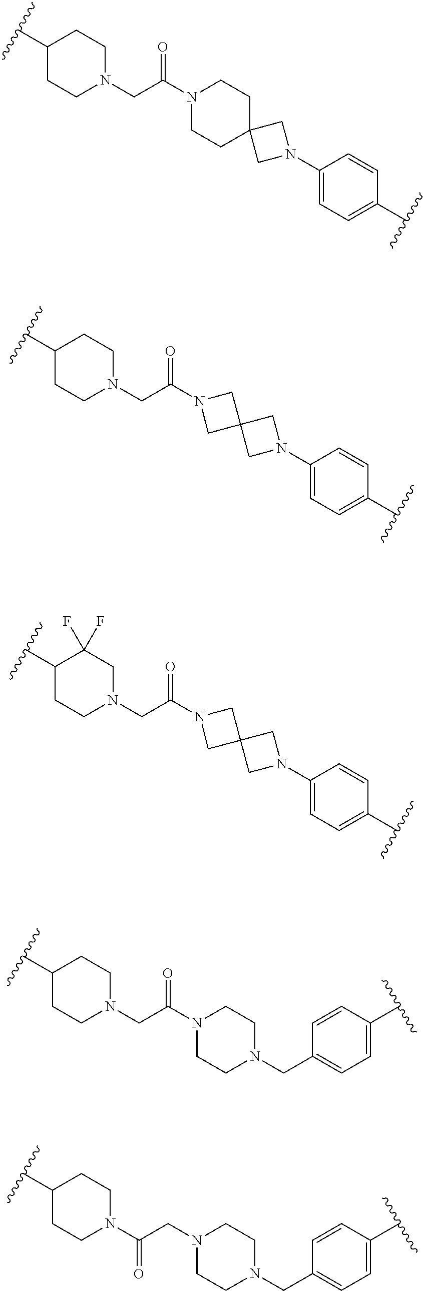 [SMILES: *c1ccc(N2CC3(CCN(C(=O)CN4CCC(*)CC4)CC3)C2)cc1 ; *c1ccc(N2CC3(CN(C(=O)CN4CCC(*)CC4)C3)C2)cc1 ; *c1ccc(N2CC3(CN(C(=O)CN4CCC(*)C(F)(F)C4)C3)C2)cc1 ; *c1ccc(CN2CCN(C(=O)CN3CCC(*)CC3)CC2)cc1 ; *c1ccc(CN2CCN(CC(=O)N3CCC(*)CC3)CC2)cc1]

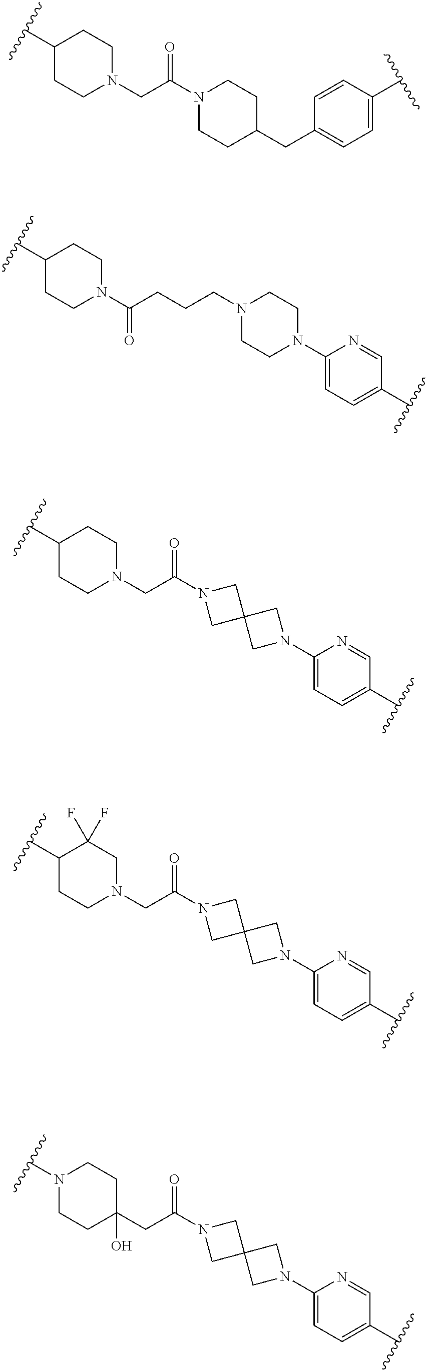 [SMILES: *c1ccc(CC2CCN(C(=O)CN3CCC(*)CC3)CC2)cc1 ; *c1ccc(N2CCN(CCCC(=O)N3CCC(*)CC3)CC2)nc1 ; *c1ccc(N2CC3(CN(C(=O)CN4CCC(*)CC4)C3)C2)nc1 ; *c1ccc(N2CC3(CN(C(=O)CN4CCC(*)C(F)(F)C4)C3)C2)nc1 ; *c1ccc(N2CC3(CN(C(=O)CC4(O)CCN(*)CC4)C3)C2)nc1]

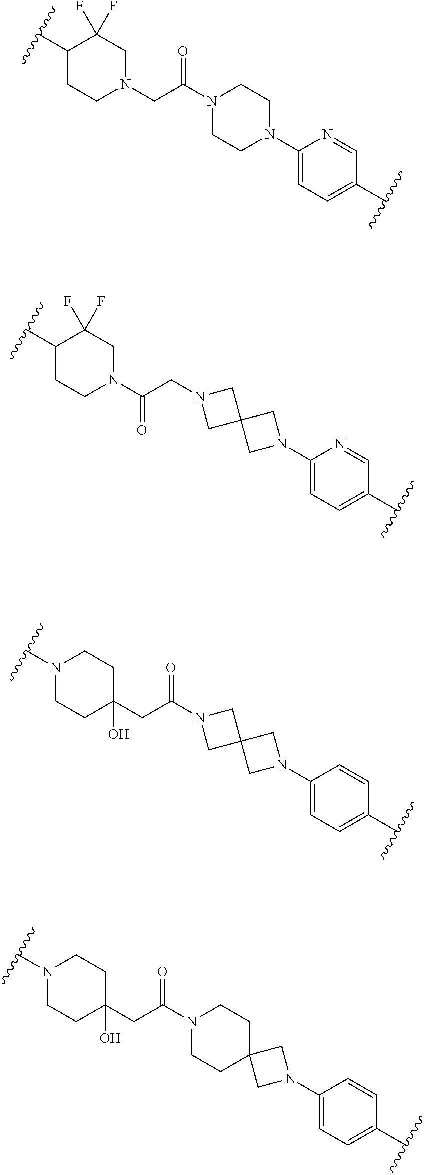 [SMILES: *c1ccc(N2CCN(C(=O)CN3CCC(*)C(F)(F)C3)CC2)nc1 ; *c1ccc(N2CC3(CN(CC(=O)N4CCC(*)C(F)(F)C4)C3)C2)nc1 ; *c1ccc(N2CC3(CN(C(=O)CC4(O)CCN(*)CC4)C3)C2)cc1 ; *c1ccc(N2CC3(CCN(C(=O)CC4(O)CCN(*)CC4)CC3)C2)cc1]

In certain embodiments L² is selected from:

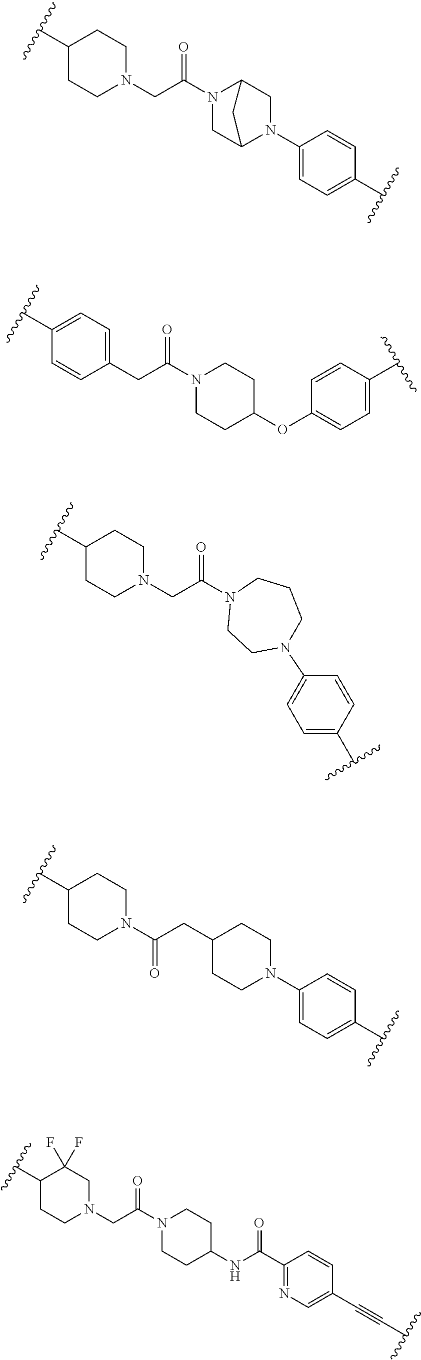 [SMILES: *c1ccc(N2CC3CC2CN3C(=O)CN2CCC(*)CC2)cc1 ; *c1ccc(CC(=O)N2CCC(Oc3ccc(*)cc3)CC2)cc1 ; *c1ccc(N2CCCN(C(=O)CN3CCC(*)CC3)CC2)cc1 ; *c1ccc(N2CCC(CC(=O)N3CCC(*)CC3)CC2)cc1 ; *C#Cc1ccc(C(=O)NC2CCN(C(=O)CN3CCC(*)C(F)(F)C3)CC2)nc1]

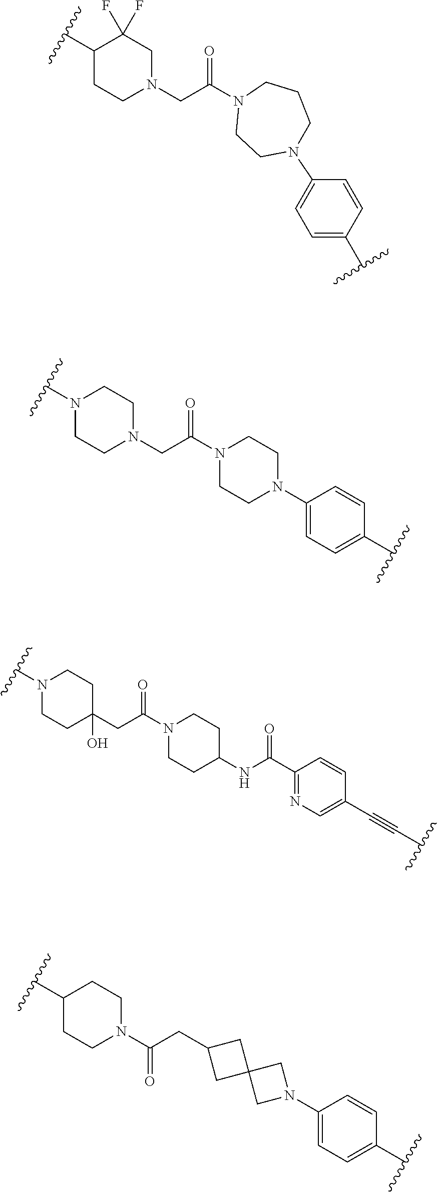 [SMILES: *c1ccc(N2CCCN(C(=O)CN3CCC(*)C(F)(F)C3)CC2)cc1 ; *c1ccc(N2CCN(C(=O)CN3CCN(*)CC3)CC2)cc1 ; *C#Cc1ccc(C(=O)NC2CCN(C(=O)CC3(O)CCN(*)CC3)CC2)nc1 ; *c1ccc(N2CC3(CC(CC(=O)N4CCC(*)CC4)C3)C2)cc1]

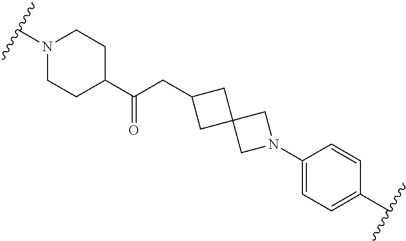 [SMILES: *c1ccc(N2CC3(CC(CC(=O)C4CCN(*)CC4)C3)C2)cc1]

In certain embodiments L² is selected from:

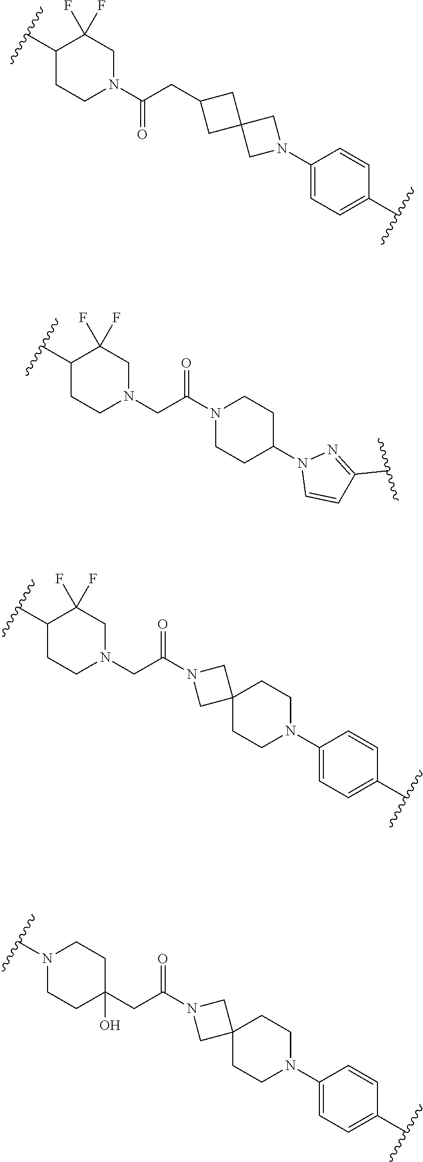 [SMILES: *c1ccc(N2CC3(CC(CC(=O)N4CCC(*)C(F)(F)C4)C3)C2)cc1 ; *c1ccn(C2CCN(C(=O)CN3CCC(*)C(F)(F)C3)CC2)n1 ; *c1ccc(N2CCC3(CC2)CN(C(=O)CN2CCC(*)C(F)(F)C2)C3)cc1 ; *c1ccc(N2CCC3(CC2)CN(C(=O)CC2(O)CCN(*)CC2)C3)cc1]

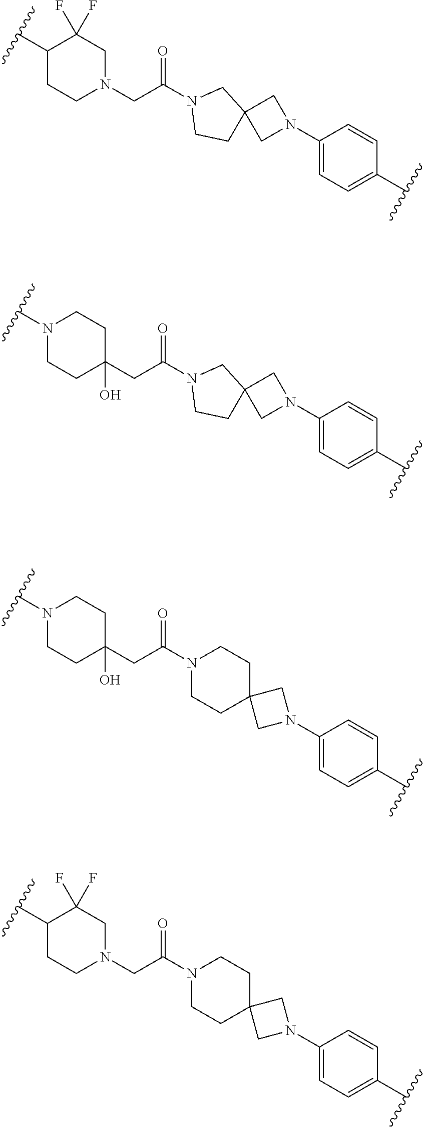 [SMILES: *c1ccc(N2CC3(CCN(C(=O)CN4CCC(*)C(F)(F)C4)C3)C2)cc1 ; *c1ccc(N2CC3(CCN(C(=O)CC4(O)CCN(*)CC4)C3)C2)cc1 ; *c1ccc(N2CC3(CCN(C(=O)CC4(O)CCN(*)CC4)CC3)C2)cc1 ; *c1ccc(N2CC3(CCN(C(=O)CN4CCC(*)C(F)(F)C4)CC3)C2)cc1]

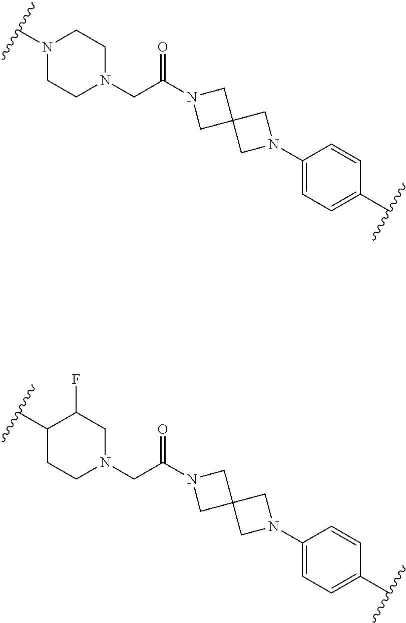 [SMILES: *c1ccc(N2CC3(CN(C(=O)CN4CCN(*)CC4)C3)C2)cc1 ; *c1ccc(N2CC3(CN(C(=O)CN4CCC(*)C(F)C4)C3)C2)cc1]

In certain embodiments L² is selected from:

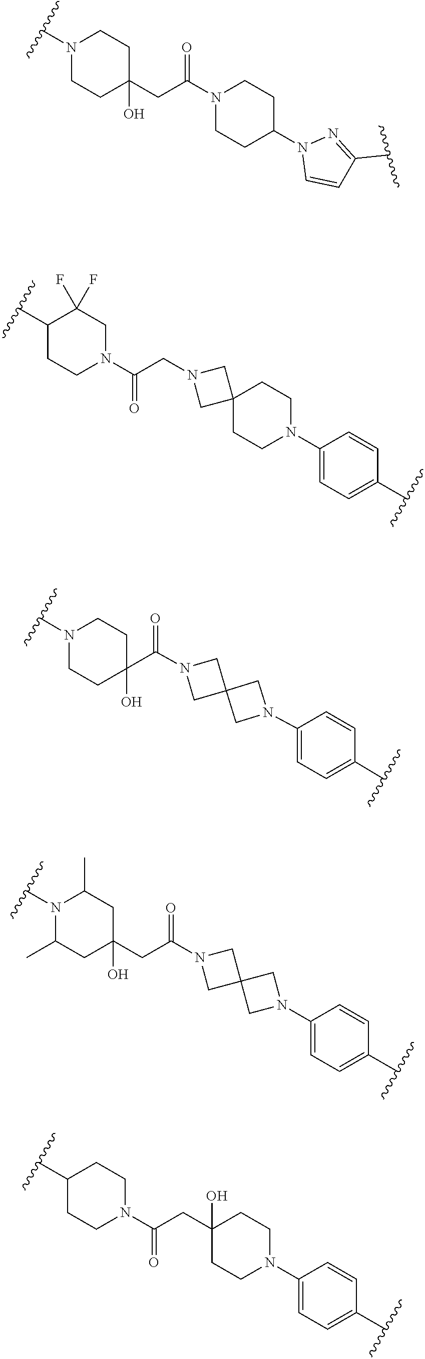 [SMILES: *c1ccn(C2CCN(C(=O)CC3(O)CCN(*)CC3)CC2)n1 ; *c1ccc(N2CCC3(CC2)CN(CC(=O)N2CCC(*)C(F)(F)C2)C3)cc1 ; *c1ccc(N2CC3(CN(C(=O)C4(O)CCN(*)CC4)C3)C2)cc1 ; *c1ccc(N2CC3(CN(C(=O)CC4(O)CC(C)N(*)C(C)C4)C3)C2)cc1 ; *c1ccc(N2CCC(O)(CC(=O)N3CCC(*)CC3)CC2)cc1]

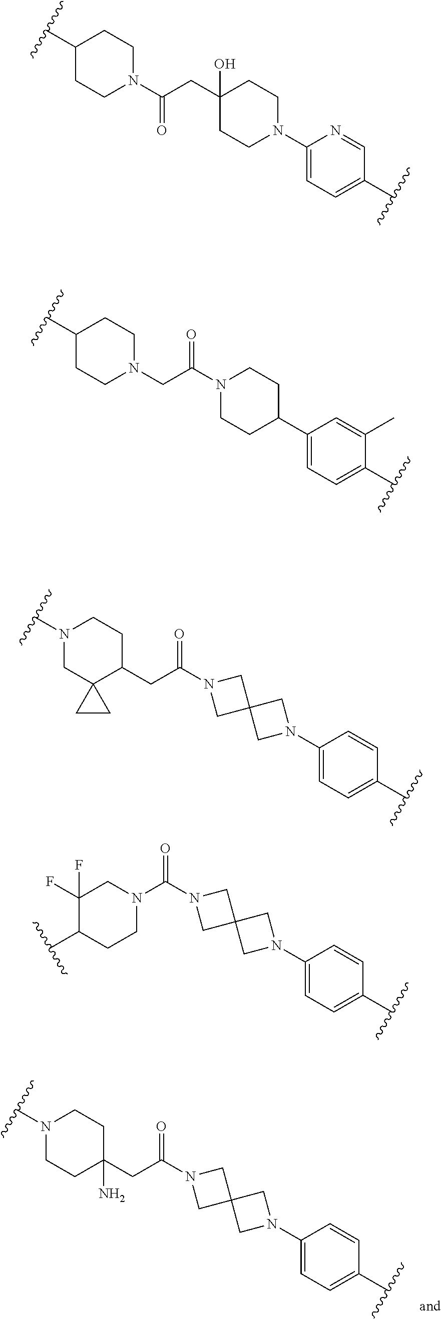 [SMILES: *c1ccc(N2CCC(O)(CC(=O)N3CCC(*)CC3)CC2)nc1 ; *c1ccc(C2CCN(C(=O)CN3CCC(*)CC3)CC2)cc1C ; *c1ccc(N2CC3(CN(C(=O)CC4CCN(*)CC45CC5)C3)C2)cc1 ; *c1ccc(N2CC3(CN(C(=O)N4CCC(*)C(F)(F)C4)C3)C2)cc1 ; *c1ccc(N2CC3(CN(C(=O)CC4(N)CCN(*)CC4)C3)C2)cc1]

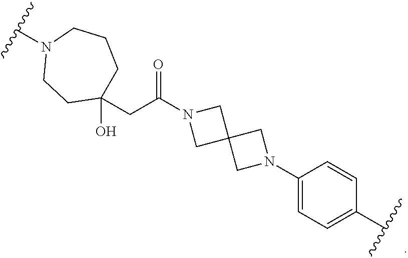 [SMILES: *c1ccc(N2CC3(CN(C(=O)CC4(O)CCCN(*)CC4)C3)C2)cc1]

In certain embodiments L² is selected from:

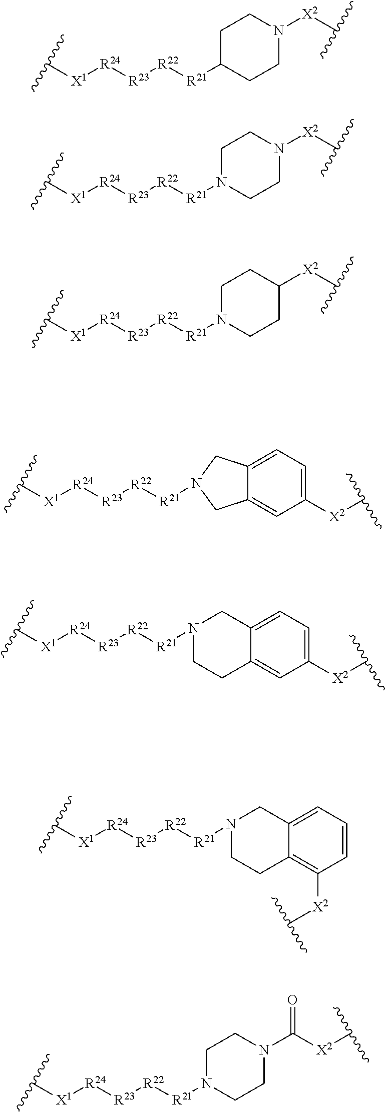

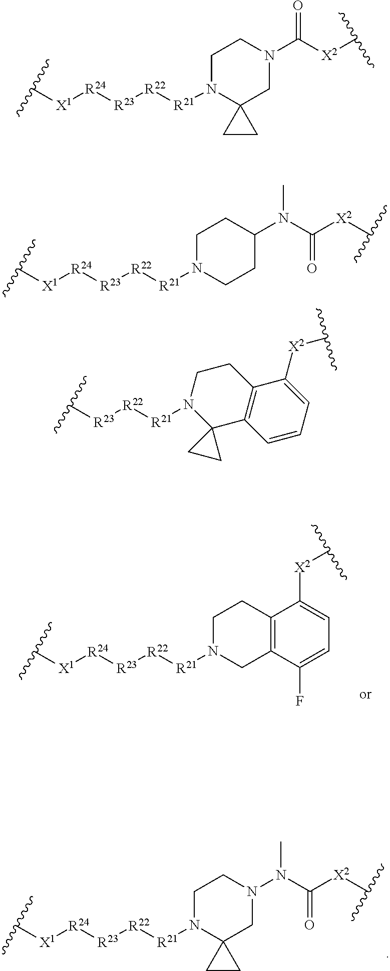

In certain embodiments L² is selected from:

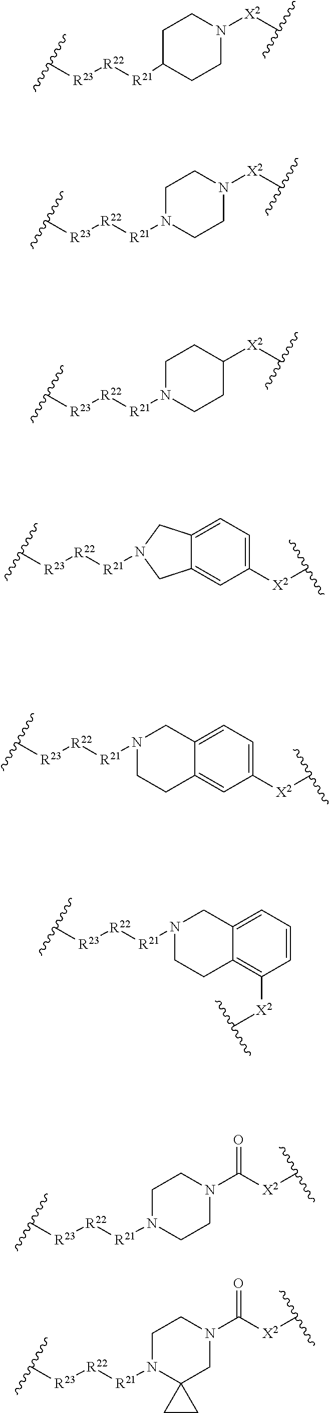

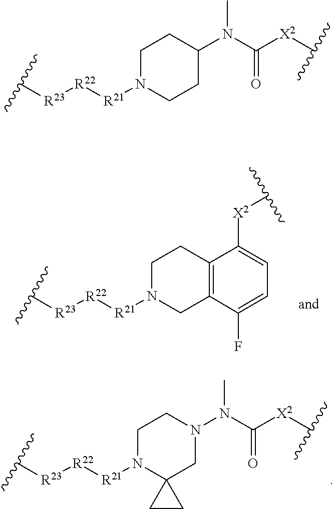

Non-limiting examples of moieties of R²⁰, R²¹, R²², R²³, and R²⁴ include:

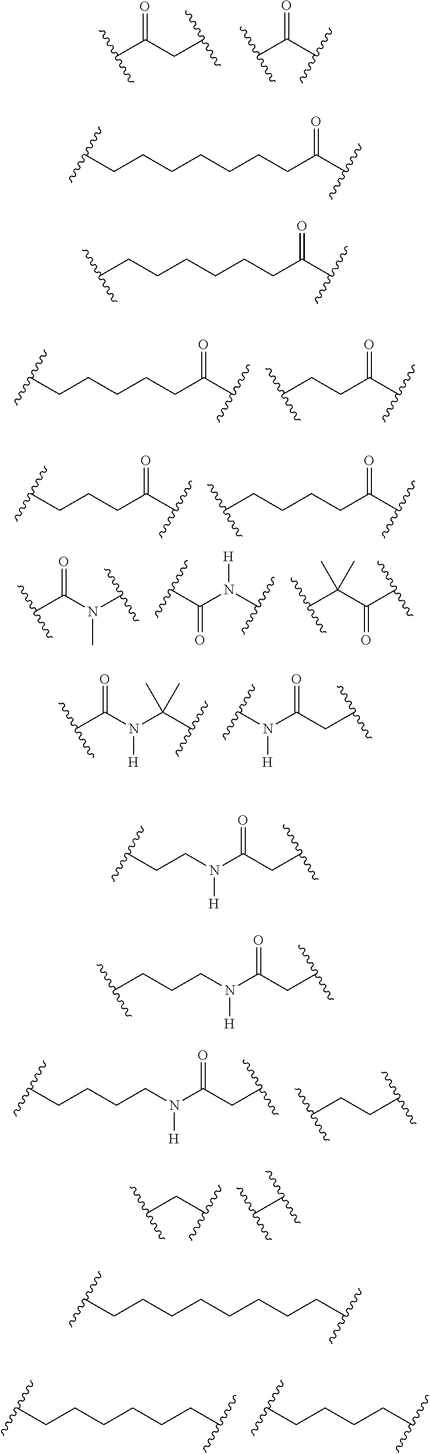

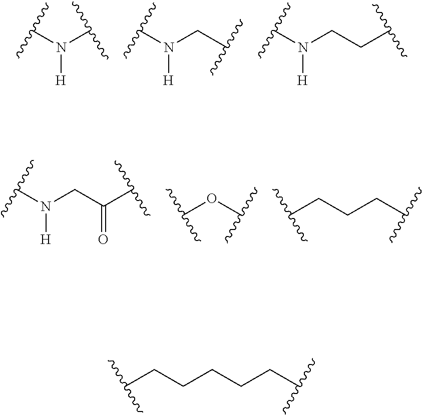

Additional non-limiting examples of moieties of R²⁰, R²¹, R²², R²³, and R²⁴ include:

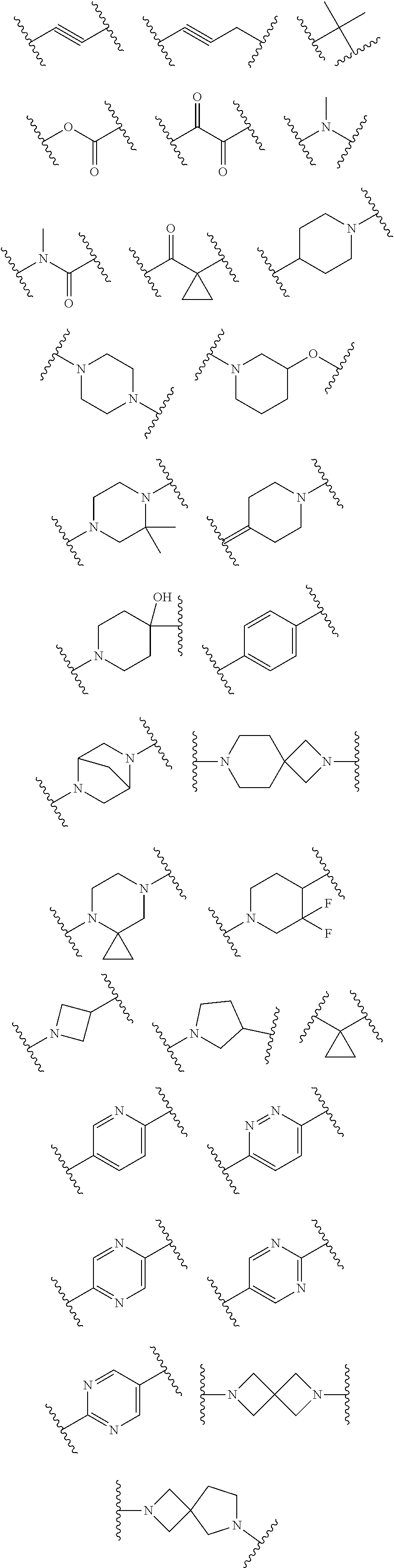

Additional non-limiting examples of moieties of R²⁰, R²¹, R²², R²³, and R²⁴ include:

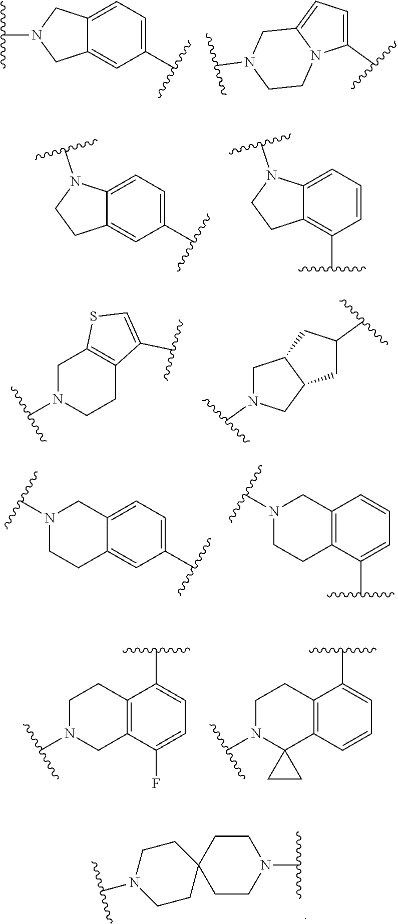

In additional embodiments, the Linker (L²) moiety is an optionally substituted (poly)ethylene glycol having at least 1, at least 2, at least 3, at least 4, at least 5, at least 6, at least 7, at least 8, at least 9, at least 10, ethylene glycol units, or optionally substituted alkyl groups interspersed with optionally substituted, O, N, S, P or Si atoms.

In certain embodiments, the Linker (L²) is flanked, substituted, or interspersed with an aryl, phenyl, benzyl, alkyl, alkylene, or heterocycle group.

In certain embodiments, the Linker (L²) may be asymmetric or symmetrical.

In certain embodiments, Linker (L²) can be a nonlinear chain, and can be, or include, aliphatic or aromatic or heteroaromatic cyclic moieties.

In any of the embodiments of the compounds described herein, the Linker group may be any suitable moiety as described herein.

In certain embodiments, the Linker (L²) is selected from the group consisting of:

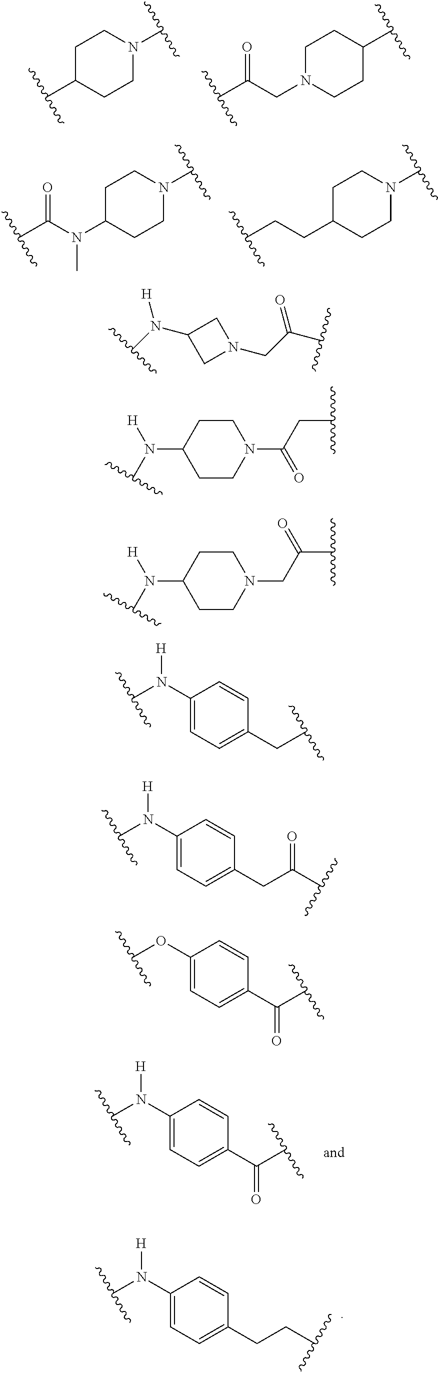

In certain embodiments, the linker (L²) is selected from the group consisting of:

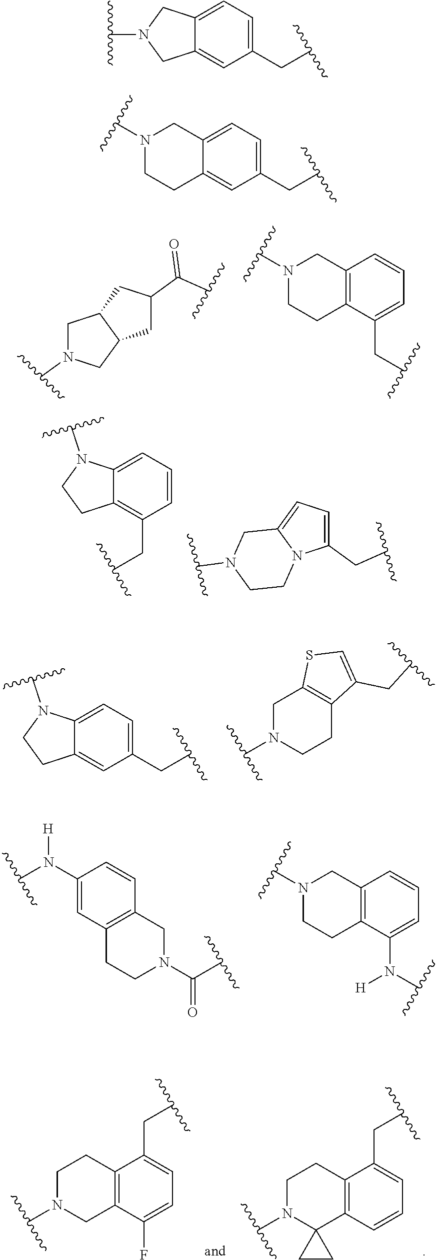

In certain embodiments, the linker (L²) is selected from the group consisting of:

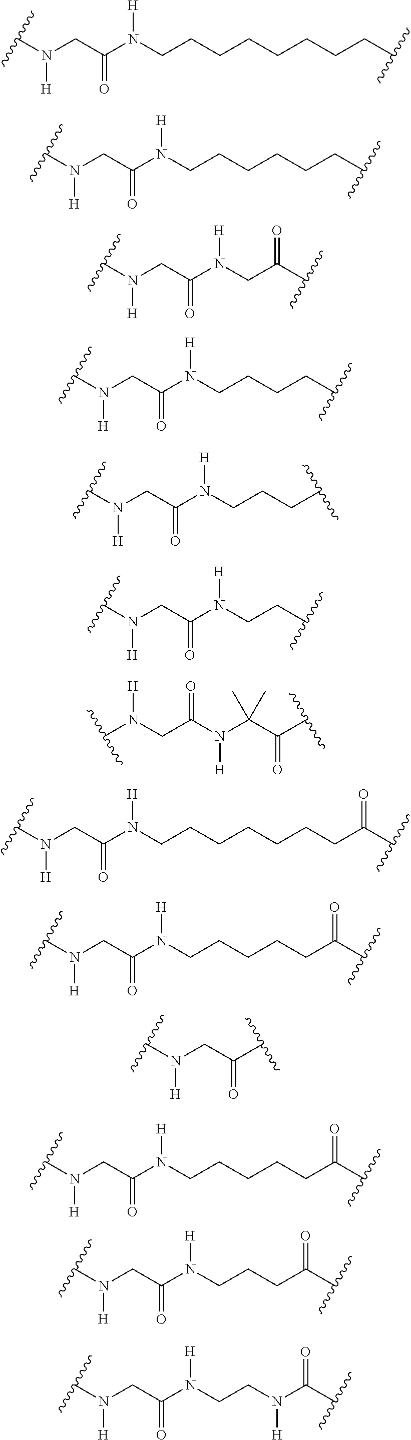

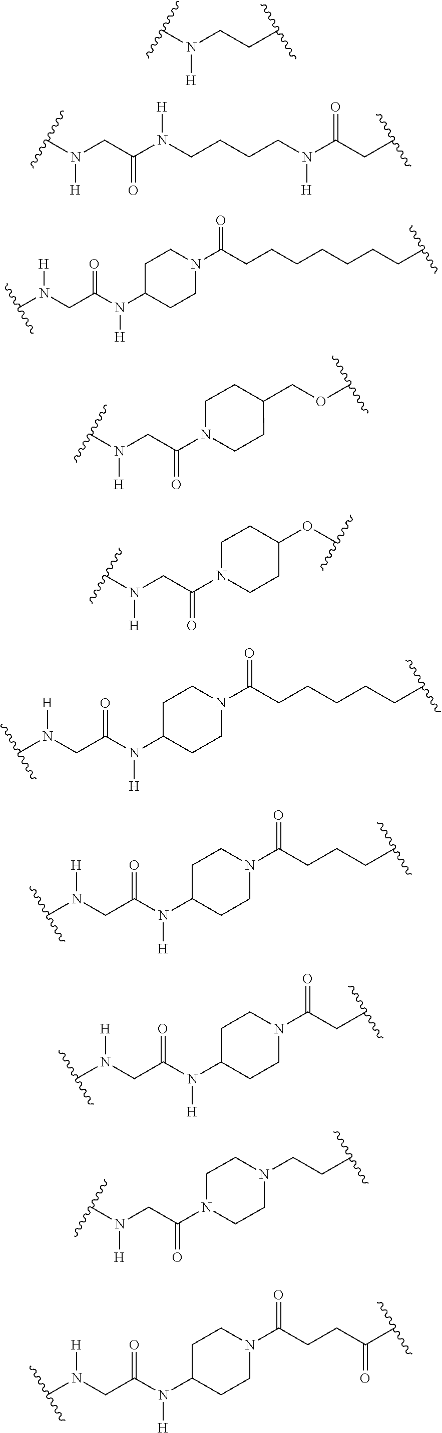

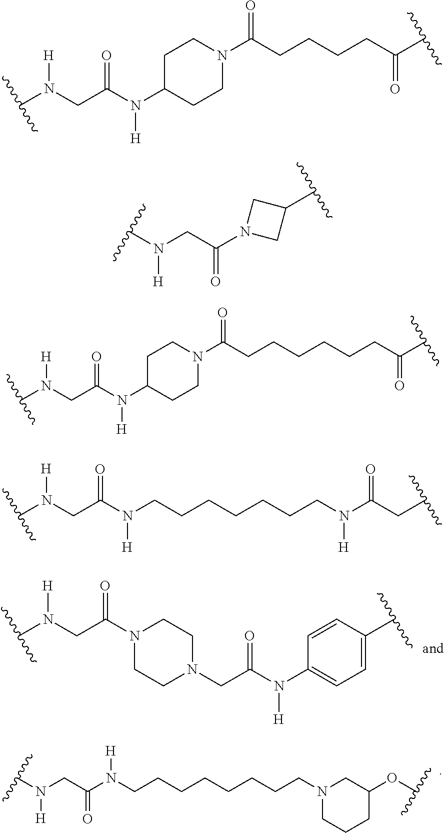

In certain embodiments, the linker (L²) is selected from the group consisting of:

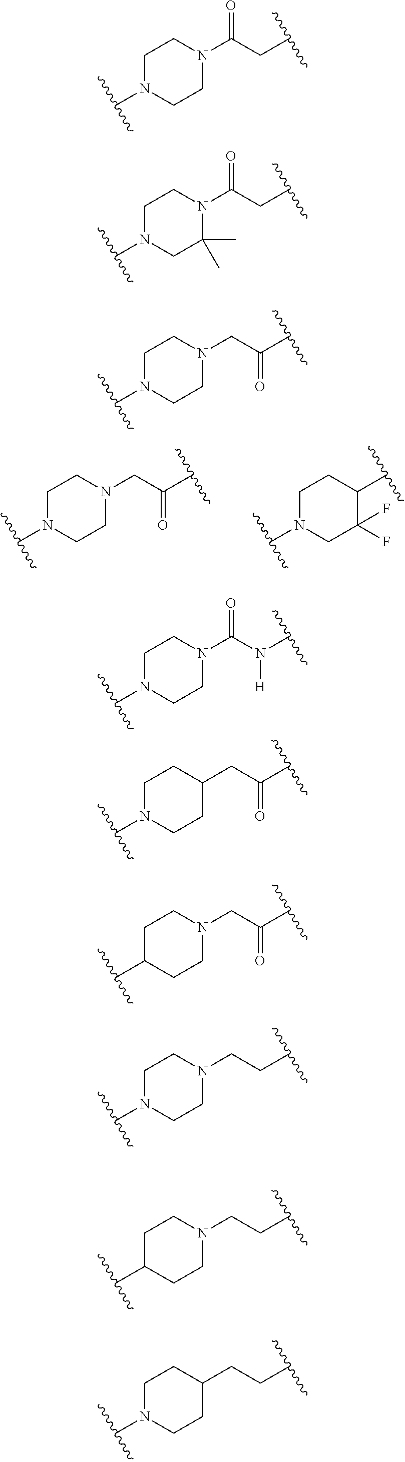

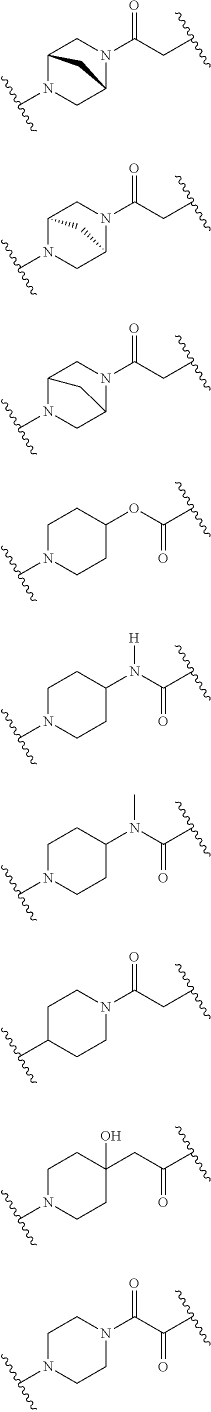

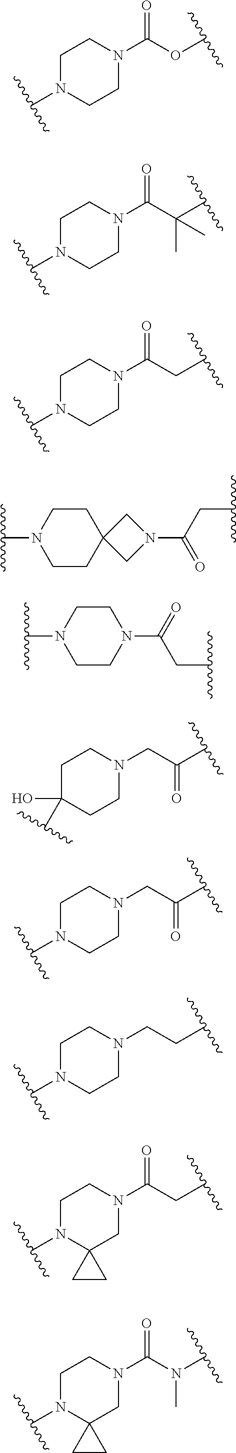

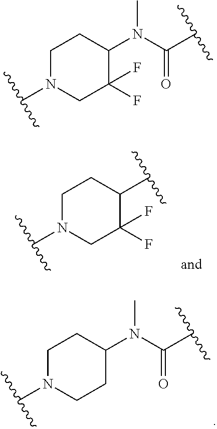 [SMILES: *C(=O)N(C)C1CCN(*)CC1(F)F ; *C1CCN(*)CC1(F)F ; *C(=O)N(C)C1CCN(*)CC1]

In certain embodiments, the linker (L²) is selected from the group consisting of:

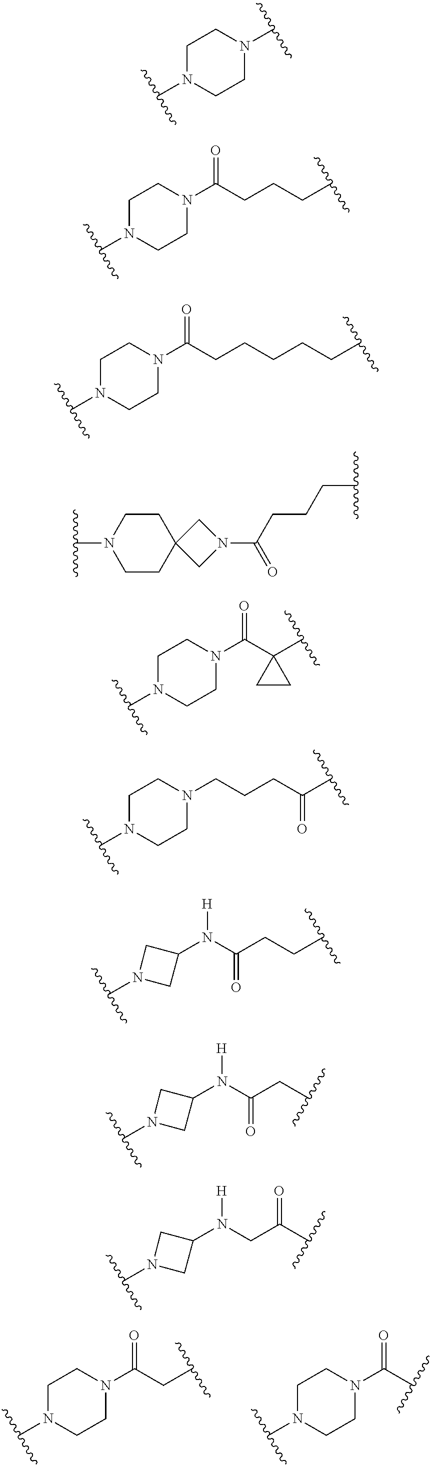

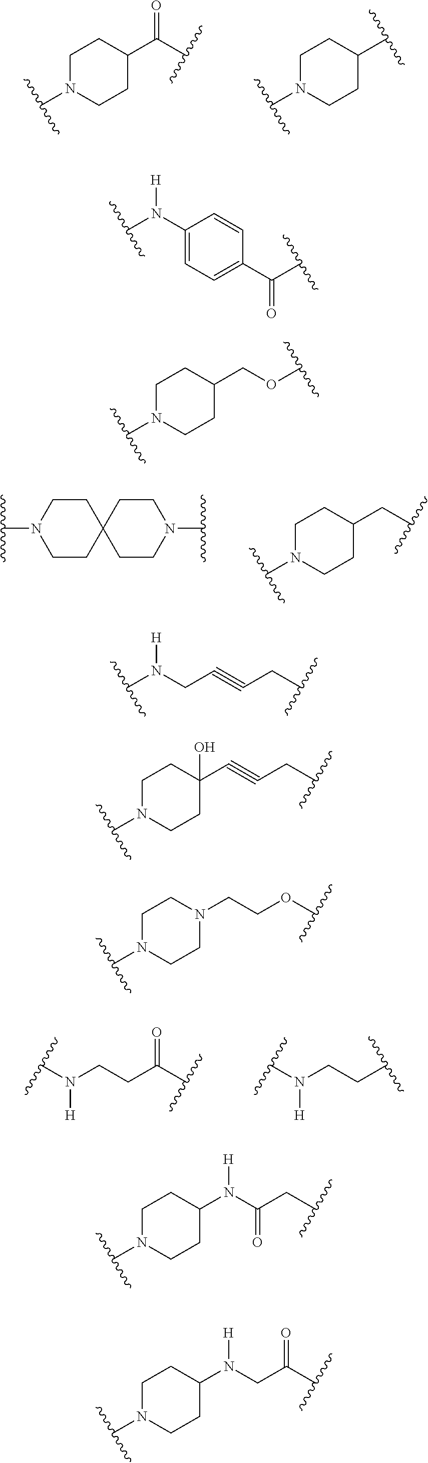

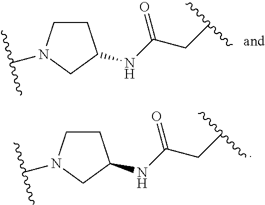

In certain embodiments, the linker (L²) is selected from the group consisting of:

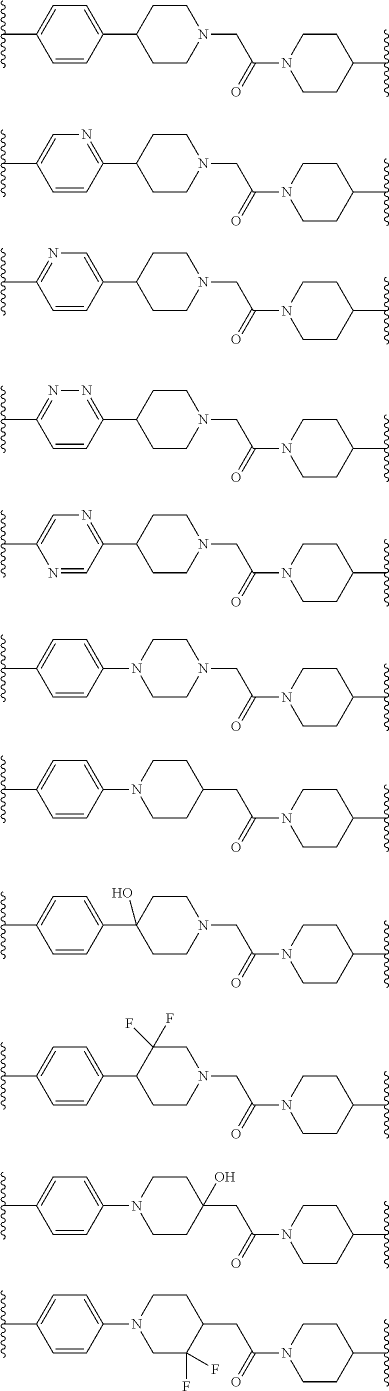

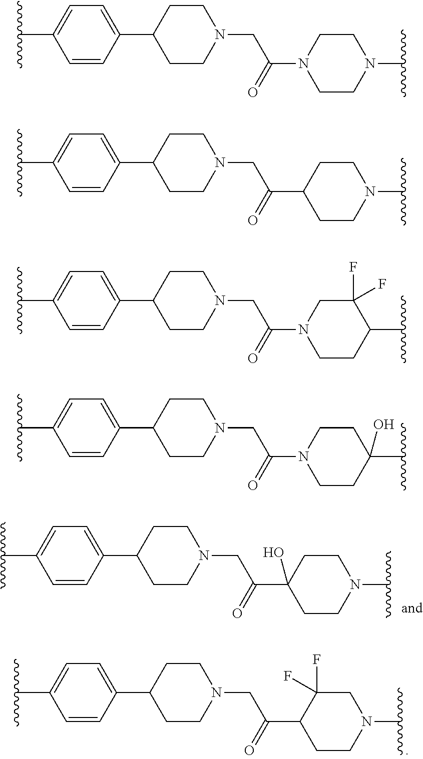 [SMILES: *c1ccc(C2CCN(CC(=O)N3CCN(*)CC3)CC2)cc1 ; *c1ccc(C2CCN(CC(=O)C3CCN(*)CC3)CC2)cc1 ; *c1ccc(C2CCN(CC(=O)N3CCC(*)C(F)(F)C3)CC2)cc1 ; *c1ccc(C2CCN(CC(=O)N3CCC(*)(O)CC3)CC2)cc1 ; *c1ccc(C2CCN(CC(=O)C3(O)CCN(*)CC3)CC2)cc1 ; *c1ccc(C2CCN(CC(=O)C3CCN(*)CC3(F)F)CC2)cc1]

In certain embodiments, the linker (L²) is selected from the group consisting of:

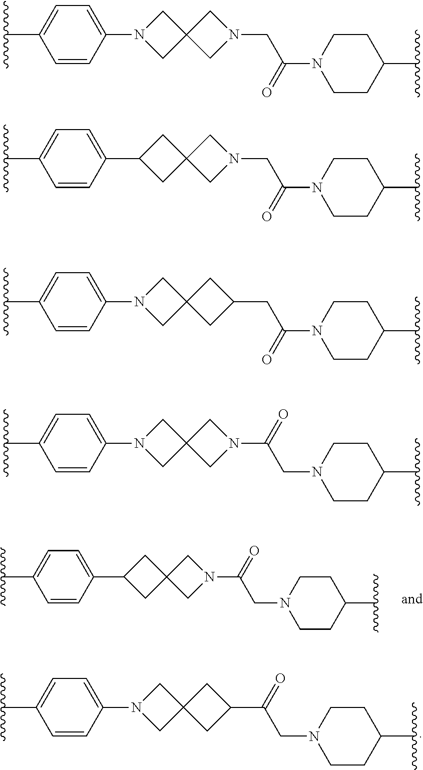 [SMILES: *c1ccc(N2CC3(CN(CC(=O)N4CCC(*)CC4)C3)C2)cc1 ; *c1ccc(C2CC3(C2)CN(CC(=O)N2CCC(*)CC2)C3)cc1 ; *c1ccc(N2CC3(CC(CC(=O)N4CCC(*)CC4)C3)C2)cc1 ; *c1ccc(N2CC3(CN(C(=O)CN4CCC(*)CC4)C3)C2)cc1 ; *c1ccc(C2CC3(C2)CN(C(=O)CN2CCC(*)CC2)C3)cc1 ; *c1ccc(N2CC3(CC(C(=O)CN4CCC(*)CC4)C3)C2)cc1]

In certain embodiments linker (L²) or a portion thereof is selected from:

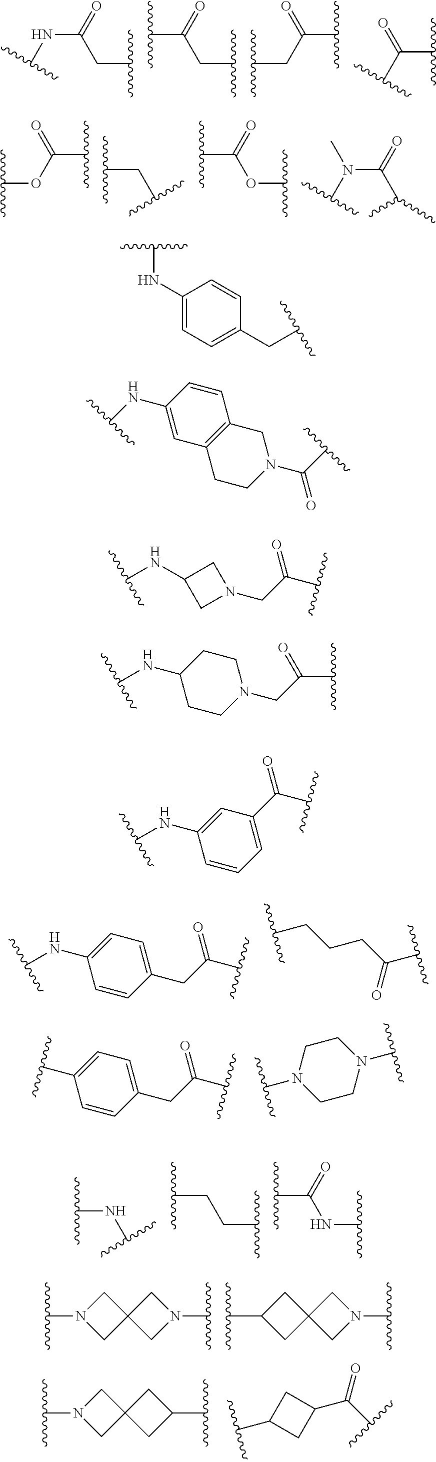

### V. Methods of Treatment

A compound described herein can be used in an effective amount to treat a patient, in need thereof, or to treat any disorder mediated by EGFR.

Another aspect described herein provides a compound as described herein, or an enantiomer, diastereomer, or stereoisomer thereof, or pharmaceutically acceptable salt, hydrate, or solvate thereof, or a pharmaceutical composition, for use in the manufacture of a medicament for treating or preventing cancer in a patient in need thereof; wherein there is a need of EGFR inhibition for the treatment or prevention of cancer.

In one aspect, a compound described herein is used to treat an EGFR mediated cancer, wherein the EGFR has mutated from the wild-type. There are a number of possibilities for EGFR mutations. In certain non-limiting embodiments, the mutation is found in exon 18, exon 19, exon 20, or exon 21, or any combination thereof. In certain nonlimiting embodiments, the mutation is at position L858, E709, G719, C797, L861, T790, or L718 or any combination thereof. In certain embodiments the mutation is a L858R, T790M, L718Q, L792H, and/or a C797S mutation or any combination thereof.

In certain aspects, the cancer has developed one or more EGFR mutations following treatment with at least one EGFR inhibitor that can be a non-covalent inhibitor (including but not limited to gefitinib, erlotinib, lapatinib or vandetanib) or a covalent inhibitor (such as afatinib, osimertinib or dacomitinib). In another aspect, the cancer has developed one or more EGFR mutations following treatment with an antibody such as cetuximab, panitumab or necitumab. In yet another aspect, the cancer has one or more EGFR mutations or non-EGFR mutations that renders the cancer intrinsically resistant to EGFR inhibitor treatment, for example, a somatic exon insertion, asomatic PIK3CA mutation, loss of PTEN expression, MET amplification, or a KRAS mutation.

In certain embodiments, a compound described herein is used to treat a cancer that is resistant to, or has acquired a resistance to, a first generation EGFR inhibitor such as erlotinib, gefitinib, and/or lapatinib. In certain embodiments, a compound described herein is used to treat a cancer that is resistant to, or has acquired a resistance to a second generation EGFR inhibitor such as afatinib and/or dacomitinib. In certain embodiments, a compound described herein is used to treat a cancer that is resistant to, or acquired a resistance to a third generation EGFR inhibitor such as osimertinib.

In one aspect, a compound described herein is used to treat an EGFR mediated cancer that has metastasized to the brain or CNS, wherein the EGFR has mutated from the wild-type. There are a number of possibilities for EGFR mutations. In certain non-limiting embodiments, the mutation is found in exon 18, exon 19, exon 20, or exon 21, or any combination thereof. In certain nonlimiting embodiments, the mutation is at position L858, E709, G719, C797, L861, T790, or L718 or any combination thereof. In certain embodiments the mutation is a L858R, T790M, L718Q, L792H, and/or a C797S mutation or any combination thereof.

In certain embodiments, a compound described herein is used to treat a cancer that has metastasized to the brain or CNS that is resistant to, or has acquired a resistance to, a first generation EGFR inhibitor such as erlotinib, gefitinib, and/or lapatinib. In certain embodiments, a compound described herein is used to treat a cancer that has metastasized to the brain or CNS that is resistant to, or has acquired a resistance to a second generation EGFR inhibitor such as afatinib and/or dacomitinib. In certain embodiments, a compound described herein is used to treat a cancer that has metastasized to the brain or CNS that is resistant to, or acquired a resistance to a third generation EGFR inhibitor such as osimertinib.

In some embodiments, the mutated EGFR protein in the diseased tissue has an L858 mutation, for example L858R.

In certain embodiments a compound described herein is used to treat a mutant EGFR mediated disorder in the brain or CNS or a mutant EGFR-mediated cancer that has metastasized to the brain or CNS, wherein EGFR has a mutation of at least one of the below listed amino acid sites, or a combination thereof. The mutation may, for example, be selected from one of the listed exemplary mutations, or may be a different mutation.

| Amino Acid | Exemplary Mutations |
|---|---|
| C797 | C797S |
| E709 | E709A, E709G, E709K, E709V |
| G719 | G719A, G719S, G719C, G719D |
| G724 | G724S |
| G119 | G119A |
| G796 | G796S, G796C |
| L718 | L718V, L718Q |
| L792 | L792H; L792V |
| L858 | L858R |
| L861 | L861Q |
| S768 | S768I |
| T790 | T790M |

In certain embodiments the mutant EGFR-mediated disorder in the brain or CNS or the mutant EGFR-mediated cancer that has metastasized to the brain or CNS has two mutations selected from the table above. In other embodiments the mutant EGFR-mediated disorder in the brain or CNS or the mutant EGFR-mediated cancer that has metastasized to the brain or CNS has three mutations selected from the table above. In other embodiments the mutant EGFR-mediated disorder in the brain or CNS or the mutant EGFR-mediated cancer that has metastasized to the brain or CNS has four or more mutations, which may optionally be selected from the table above.

In certain embodiments the mutant EGFR-mediated disorder in the brain or CNS or the mutant EGFR-mediated cancer that has metastasized to the brain or CNS has an L858R mutation and one additional mutation which may optionally be selected from the table above. In some of these embodiments the mutant EGFR-mediated disorder in the brain or CNS or the mutant EGFR-mediated cancer that has metastasized to the brain or CNS has an L858R mutation and two additional mutation that may optionally be selected from the table above. In other embodiments the mutant EGFR-mediated disorder in the brain or CNS or the mutant EGFR-mediated cancer that has metastasized to the brain or CNS has a L858R mutation and three additional mutation that may optionally be selected from the table above.

In certain embodiments the mutant EGFR-mediated disorder in the brain or CNS or the mutant EGFR-mediated cancer that has metastasized to the brain or CNS has a T790M mutation and one additional mutation optionally selected from the table above. In other embodiments the mutant EGFR-mediated disorder in the brain or CNS or the mutant EGFR-mediated cancer that has metastasized to the brain or CNS has a T790M mutation and two additional mutation optionally selected from the table above. In other embodiments the mutant EGFR-mediated disorder in the brain or CNS or the mutant EGFR-mediated cancer that has metastasized to the brain or CNS has a T790M mutation and three additional mutation optionally selected from the table above.

In certain embodiments the mutant EGFR-mediated disorder in the brain or CNS or the mutant EGFR-mediated cancer that has metastasized to the brain or CNS has a L718Q mutation and one additional mutation optionally selected from the table above. In other embodiments the mutant EGFR-mediated disorder in the brain or CNS or the mutant EGFR-mediated cancer that has metastasized to the brain or CNS has a L718Q mutation and two additional mutation optionally selected from the table above. In other embodiments the mutant EGFR-mediated disorder in the brain or CNS or the mutant EGFR-mediated cancer that has metastasized to the brain or CNS has a L718Q mutation and three additional mutation optionally selected from the table above.

In certain embodiments the mutant EGFR-mediated disorder in the brain or CNS or the mutant EGFR-mediated cancer that has metastasized to the brain or CNS has a mutation of S768I, L718V, L792H, L792V, G796S, G796C, G724S, and/or G719A.

In certain embodiments, a compound described herein is used to treat a mutant EGFR-mediated disorder in the brain or CNS or a mutant EGFR-mediated cancer that has metastasized to the brain or CNS that has a frameshift mutation, for example a short in-frame deletion. In certain embodiments, a compound described herein is used to treat a mutant EGFR-mediated disorder in the brain or CNS or a mutant EGFR-mediated cancer that has metastasized to the brain or CNS wherein the EGFR has an exon 19 deletion. In certain embodiments, the exon 19 deletion is a deletion which includes the amino acids LREA (L747-A750). In certain embodiments, the exon 19 deletion is a deletion which includes the amino acids ELREA (E746-A750).

In certain embodiments a compound described herein is used to treat a mutant EGFR-mediated disorder in the brain or CNS or a mutant EGFR-mediated cancer that has metastasized to the brain or CNS wherein the EGFR has an L858R mutation in exon 21.

In certain embodiments a compound described herein is more active against a disorder driven by a mutated EGFR than wild-type EGFR.

In certain embodiments, a compound described herein is used to treat EGFR-mediated cancer that has metastasized to the brain or CNS wherein the EGFR has one or more exon 18 deletions.

In certain embodiments a compound described herein is used to treat a mutant EGFR-mediated disorder in the brain or CNS or a mutant EGFR-mediated cancer that has metastasized to the brain or CNS with a E709 mutation, for example E709A, E709G, E709K, or E709V.

In certain embodiments a compound described herein is used to treat a mutant EGFR-mediated disorder in the brain or CNS or a mutant EGFR-mediated cancer that has metastasized to the brain or CNS with a L718 mutation, for example L718Q.

In certain embodiments a compound described herein is used to treat a mutant EGFR-mediated disorder in the brain or CNS or a mutant EGFR-mediated cancer that has metastasized to the brain or CNS with a G719 mutation, for example G719S, G719A, G719C, or G719D.

In certain embodiments, a compound described herein is used to treat a mutant EGFR-mediated disorder in the brain or CNS or a mutant EGFR-mediated cancer that has metastasized to the brain or CNS wherein the EGFR has one or more exon 19 insertions and/or one or more exon 20 insertions.

In certain embodiments, a compound described herein is used to treat a S7681 mutant EGFR-mediated disorder in the brain or CNS or a S7681 mutant EGFR-mediated cancer that has metastasized to the brain or CNS. In certain embodiments a compound described herein is used to treat a L861Q mutant EGFR-mediated disorder in the brain or CNS or a EGFR L861Q mutant EGFR-mediated cancer that has metastasized to the brain or CNS. In certain embodiments, a compound described herein is used to treat C797S mutant EGFR-mediated cancer that has metastasized to the brain or CNS.

In certain embodiments a compound described herein is used to treat a L858R-T790M mutant EGFR-mediated disorder in the brain or CNS.

In certain embodiments a compound described herein is used to treat a L858R-L718Q mutant EGFR-mediated disorder in the brain or CNS.

In certain embodiments a compound described herein is used to treat a L858R-L792H mutant EGFR-mediated disorder in the brain or CNS.

In certain embodiments a compound described herein is used to treat a L858R-C797S mutant EGFR-mediated disorder in the brain or CNS.

In certain embodiments a compound described herein is used to treat a L858R-T790M mutant EGFR-mediated cancer that has metastasized to the brain or CNS.

In certain embodiments a compound described herein is used to treat a L858R-L718Q mutant EGFR-mediated cancer that has metastasized to the brain or CNS.

In certain embodiments a compound described herein is used to treat a L858R-L792H mutant EGFR-mediated cancer that has metastasized to the brain or CNS.

In certain embodiments a compound described herein is used to treat a L858R-C797S mutant EGFR-mediated cancer that has metastasized to the brain or CNS.

In certain embodiments, the EGFR mediated cancer that has metastasized to the brain or CNS is a hematological cancer.

In certain embodiments, the EGFR mediated cancer that has metastasized to the brain or CNS is acute myelogenous leukemia (AML), acute lymphoblastic leukemia (ALL), lymphoblastic T-cell leukemia, chronic myelogenous leukemia (CML), chronic lymphocytic leukemia (CLL), hairy-cell leukemia, chronic neutrophilic leukemia (CNL), acute lymphoblastic T-cell leukemia, acute monocytic leukemia, plasmacytoma, immunoblastic large cell leukemia, mantle cell leukemia, multiple myeloma, megakaryoblastic leukemia, acute megakaryocytic leukemia, promyelocytic leukemia, mixed lineage leukemia (MLL), erythroleukemia, malignant lymphoma, Hodgkins lymphoma, non-Hodgkins lymphoma, lymphoblastic T-cell lymphoma, Burkitt's lymphoma, follicular lymphoma, B cell acute lymphoblastic leukemia, diffuse large B cell lymphoma, Myc and B-Cell Leukemia (BCL)2 and/or BCL6 rearrangements/overexpression [double- and triple-hit lymphoma], myelodysplastic/myeloproliferative neoplasm, mantle cell lymphoma including bortezomib resistant mantle cell lymphoma.

Additional EGFR mediated cancer that has metastasized to the brain or CNS that can be treated with the compounds described herein include, but are not limited to lung cancers, including small cell lung cancer (SCLC) and non-small cell lung cancer (NSCLC), breast cancers including inflammatory breast cancer, ER-positive breast cancer including tamoxifen resistant ER-positive breast cancer, and triple negative breast cancer, colon cancers, midline carcinomas, liver cancers, renal cancers, prostate cancers including castrate resistant prostate cancer (CRPC), brain cancers including gliomas, glioblastomas, neuroblastoma, and medulloblastoma including MYC-amplified medulloblastoma, colorectal cancers, Wilm's tumor, Ewing's sarcoma, rhabdomyosarcomas, ependymomas, head and neck cancers, melanomas, squamous cell carcinomas, ovarian cancers, pancreatic cancers including pancreatic ductal adenocarcinomas (PDAC) and pancreatic neuroendocrine tumors (PanNET), osteosarcomas, giant cell tumors of bone, thyroid cancers, bladder cancers, urothelial cancers, vulval cancers, cervical cancers, endometrial cancers, mesotheliomas, esophageal cancers, salivary gland cancers, gastric cancer, nasopharangeal cancers, buccal cancers, cancers of the mouth, GIST (gastrointestinal stromal tumors), NUT-midline carcinomas, testicular cancers, squamous cell carcinomas, hepatocellular carcinomas (HCC), MYCN driven solid tumors, and NUT midline carcinomas (NMC).

In further embodiments, the cancer that has metastasized to the brain or CNS is sarcoma of the bones, muscles, tendons, cartilage, nerves, fat, or blood vessels.

In further embodiments, the cancer that has metastasized to the brain or CNS is soft tissue sarcoma, bone sarcoma, or osteosarcoma.

In further embodiments, the cancer that has metastasized to the brain or CNS is angiosarcoma, fibrosarcoma, liposarcoma, leiomyosarcoma, Karposi's sarcoma, osteosarcoma, gastrointestinal stromal tumor, synovial sarcoma, pleomorphic sarcoma, chondrosarcoma, Ewing's sarcoma, reticulum cell sarcoma, meningiosarcoma, botryoid sarcoma, rhabdomyosarcoma, or embryonal rhabdomyosarcoma.

In certain embodiments the cancer that has metastasized to the brain or CNS is a bone, muscle, tendon, cartilage, nerve, fat, or blood vessel sarcoma.

In further embodiments, the cancer that has metastasized to the brain or CNS is multiple myeloma.

In certain embodiments a compound described herein or a pharmaceutically acceptable salt thereof is used as a medicament in therapeutic and/or prophylactic treatment of a patient with EGFR activating mutations as determined by next-generation sequencing (NGS), suffering from cancer, in particular non-small-cell lung cancer, comprising determining the EGFR activating mutations status in said patient and then administering the compound described herein, or a pharmaceutically acceptable salt thereof, to said patient.

In other embodiments, the cancer that has metastasized to the brain or CNS is selected from lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, solid tumors, hematological cancers or solid cancers.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers, such as oral, laryngeal, nasopharyngeal and esophageal, genitourinary cancers, such as prostate, bladder, renal, uterine, ovarian, testicular, lung cancer, such as small-cell and non-small cell, breast cancer, pancreatic cancer, melanoma and other skin cancers, stomach cancer, brain tumors, tumors related to Gorlin's syndrome, such as medulloblastoma or meningioma, and liver cancer.

Additional exemplary forms of cancer include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds described herein may be useful in preventing, treating and studying are, for example, colon carcinoma, familiary adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the application, the present application provides for the use of one or more compound as described herein, in the manufacture of a medicament for the treatment of cancer, including without limitation the various types of cancer disclosed herein.

In some embodiments, a compound described herein is useful for treating cancer which has metastasized to the brain or CNS, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compound as described herein is useful for treating hematopoietic disorders, in particular, acute-myelogenous leukemia (AML), chronic-myelogenous leukemia (CML), acute-promyelocytic leukemia, and acute lymphocytic leukemia (ALL).

In certain embodiments, a compound described herein or its corresponding pharmaceutically acceptable salt, or isotopic derivative, as described herein can be used in an effective amount to treat a host with a cancer that has metastasized to the brain or CNS, for example a human, wherein the cancer that has metastasized to the brain or CNS is selected from a lymphoma or lymphocytic or myelocytic proliferation disorder or abnormality. For example, a compound as described herein can be administered to a host suffering from a Hodgkin's Lymphoma or a Non-Hodgkin's Lymphoma. For example, the host can be suffering from a Non-Hodgkin's Lymphoma such as, but not limited to: an AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); diffuse small-cleaved cell lymphoma (DSCCL); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; Langerhans cell histiocytosis; or Waldenstrom's Macroglobulinemia.

In another embodiment, a compound described herein or its corresponding pharmaceutically acceptable salt, or isotopic derivative, as described herein can be used in an effective amount to treat a patient, for example a human, with a cancer that has metastasized to the brain or CNS selected from Hodgkin's lymphoma, such as, but not limited to: Nodular Sclerosis Classical Hodgkin's Lymphoma (CHL); Mixed Cellularity CHL; Lymphocyte-depletion CHL; Lymphocyte-rich CHL; Lymphocyte Predominant Hodgkin's Lymphoma; or Nodular Lymphocyte Predominant HL.

This application further embraces the treatment or prevention of cell proliferative disorders such as hyperplasias, dysplasias and pre-cancerous lesions. Dysplasia is the earliest form of pre-cancerous lesion recognizable in a biopsy by a pathologist. The compounds may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

As degraders of EGFR protein, the compounds and compositions of this application are also useful in biological samples. One aspect of the application is inhibiting protein activity in a biological sample, which method comprises contacting said biological sample with a compound or composition as described herein. The term “biological sample”, as used herein, means an in vitro or an ex vivo sample, including, without limitation, cell cultures or extracts thereof; biopsied material obtained from a mammal or extracts thereof; and blood, saliva, urine, feces, semen, tears, or other body fluids or extracts thereof. Inhibition of protein activity in a biological sample is useful for a variety of purposes that are known to one of skill in the art. Examples of such purposes include, but are not limited to, blood transfusion, organ-transplantation, and biological specimen storage.

Another aspect of this application is the study of EGFR protein in biological and pathological phenomena; the study of intracellular signal transduction pathways mediated by such proteins; and the comparative evaluation of new protein inhibitors. Examples of such uses include, but are not limited to, biological assays such as enzyme assays and cell-based assays.

In accordance with the foregoing, the present application further provides a method for preventing or treating any of the diseases or disorders described above in a patient in need of such treatment, which method comprises administering to said patient a therapeutically effective amount of a compound as described herein, or an enantiomer, diastereomer, or stereoisomer thereof, or pharmaceutically acceptable salt, hydrate, or solvate thereof. For any of the above uses, the required dosage will vary depending on the mode of administration, the particular condition to be treated and the effect desired.

### VI. Combination Therapy

The disclosed compounds described herein can be used in an effective amount alone or in combination with another compound described herein or another bioactive agent or second therapeutic agent to treat a patient such as a human with an EGFR-mediated cancer that has metastasized to the brain or CNS, including but not limited to those described herein.

The term “bioactive agent” is used to describe an agent, other than the selected compound according to the present invention, which can be used in combination or alternation with a compound described herein to achieve a desired result of therapy. In certain embodiments, the compound described herein and the bioactive agent are administered in a manner that they are active in vivo during overlapping time periods, for example, have time-period overlapping Cmax, Tmax, AUC or another pharmacokinetic parameter. In another embodiment, the compound described herein and the bioactive agent are administered to a patient in need thereof that do not have overlapping pharmacokinetic parameter, however, one has a therapeutic impact on the therapeutic efficacy of the other.

In one aspect of this embodiment, the bioactive agent is an immune modulator, including but not limited to a checkpoint inhibitor, including as non-limiting examples, a PD-1 inhibitor, PD-L1 inhibitor, PD-L2 inhibitor, CTLA-4 inhibitor, LAG-3 inhibitor, TIM-3 inhibitor, V-domain Ig suppressor of T-cell activation (VISTA) inhibitors, small molecule, peptide, nucleotide, or other inhibitor. In certain aspects, the immune modulator is an antibody, such as a monoclonal antibody.

PD-1 inhibitors that blocks the interaction of PD-1 and PD-L1 by binding to the PD-1 receptor, and in turn inhibit immune suppression include, for example, nivolumab (OPDIVO®), pembrolizumab) (KEYTRUDA®), pidilizumab, AMP-224 (AstraZeneca and MedImmune), PF-06801591 (Pfizer), MEDI0680 (AstraZeneca), PDR001 (Novartis), REGN2810 (Regeneron), SHR-12-1 (Jiangsu Hengrui Medicine Company and Incyte Corporation), TSR-042 (GlaxoSmithKline plc), and the PD-L1/VISTA inhibitor CA-170 (Curis Inc.). PD-L1 inhibitors that block the interaction of PD-1 and PD-L1 by binding to the PD-L1 receptor, and in turn inhibits immune suppression, include for example, atezolizumab (TECENTRIQ®), durvalumab (AstraZeneca and MedImmune), KN035 (Alphamab Co. Ltd.), and BMS-936559 (Bristol-Myers Squibb). CTLA-4 checkpoint inhibitors that bind to CTLA-4 and inhibits immune suppression include, but are not limited to, ipilimumab, tremelimumab (AstraZeneca and MedImmune), AGEN1884 and AGEN2041 (Agenus). LAG-3 checkpoint inhibitors include, but are not limited to, BMS-986016 (Bristol-Myers Squibb), GSK2831781 (GlaxoSmithKline plc), IMP321 (Prima BioMed), LAG525 (Novartis), and the dual PD-1 and LAG-3 inhibitor MGD013 (MacroGenics). An example of a TIM-3 inhibitor is TSR-022 (GlaxoSmithKline plc).

In certain embodiments the checkpoint inhibitor is selected from nivolumab (OPDIVO®); pembrolizumab (KEYTRUDA®); and pidilizumab/CT-011, MPDL3280A/RG7446; MEDI4736; MSB0010718C; BMS 936559, a PDL2/lg fusion protein such as AMP 224 or an inhibitor of B7-H3 (e.g., MGA271), B7-H4, BTLA, HVEM, TIM3, GALS, LAG 3, VISTA, KIR, 2B4, CD160, CGEN-15049, CHK 1, CHK2, A2aR, B-7 family ligands, or a combination thereof.

In yet another embodiment, one of the active compounds described herein can be administered in an effective amount for the treatment of abnormal tissue of the female reproductive system such as breast, ovarian, endometrial, or uterine cancer, in combination or alternation with an effective amount of an estrogen inhibitor including, but not limited to, a SERM (selective estrogen receptor modulator), a SERD (selective estrogen receptor degrader), a complete estrogen receptor degrader, or another form of partial or complete estrogen antagonist or agonist. Partial anti-estrogens like raloxifene and tamoxifen retain some estrogen-like effects, including an estrogen-like stimulation of uterine growth, and also, in some cases, an estrogen-like action during breast cancer progression which actually stimulates tumor growth. In contrast, fulvestrant, a complete anti-estrogen, is free of estrogen-like action on the uterus and is effective in tamoxifen-resistant tumors.

Non-limiting examples of anti-estrogen compounds are provided in WO 2014/19176 assigned to Astra Zeneca, WO2013/090921, WO 2014/203129, WO 2014/203132, and US2013/0178445 assigned to Olema Pharmaceuticals, and U.S. Pat. Nos. 9,078,871, 8,853,423, and 8,703, 810, as well as US 2015/0005286, WO 2014/205136, and WO 2014/205138.

Additional non-limiting examples of anti-estrogen compounds include: SERMS such as anordrin, bazedoxifene, broparestriol, chlorotrianisene, clomiphene citrate, cyclofenil, lasofoxifene, ormeloxifene, raloxifene, tamoxifen, toremifene, and fulvestratnt; aromatase inhibitors such as aminoglutethimide, testolactone, anastrozole, exemestane, fadrozole, formestane, and letrozole; and antigonadotropins such as leuprorelin, cetrorelix, allylestrenol, chloromadinone acetate, cyproterone acetate, delmadinone acetate, dydrogesterone, medroxyprogesterone acetate, megestrol acetate, nomegestrol acetate, norethisterone acetate, progesterone, and spironolactone.

Other estrogenic ligands that can be used according to the present invention are described in U.S. Pat. Nos. 4,418,068; 5,478,847; 5,393,763; and 5,457,117, WO2011/156518, U.S. Pat. Nos. 8,455,534 and 8,299,112, 9,078,871; 8,853,423; 8,703,810; US 2015/0005286; and WO 2014/205138, US2016/0175289, US2015/0258080, WO 2014/191726, WO 2012/084711; WO 2002/013802; WO 2002/004418; WO 2002/003992; WO 2002/003991; WO 2002/003990; WO 2002/003989; WO 2002/003988; WO 2002/003986; WO 2002/003977; WO 2002/003976; WO 2002/003975; WO 2006/078834; U.S. Pat. No. 6,821,989; US 2002/0128276; U.S. Pat. No. 6,777,424; US 2002/0016340; U.S. Pat. Nos. 6,326,392; 6,756,401; US 2002/0013327; U.S. Pat. Nos. 6,512,002; 6,632,834; US 2001/0056099; U.S. Pat. Nos. 6,583,170; 6,479,535; WO 1999/024027; U.S. Pat. No. 6,005,102; EP 0802184; U.S. Pat. Nos. 5,998,402; 5,780,497, 5,880,137, WO 2012/048058 and WO 2007/087684.

In another embodiment, active compounds described herein can be administered in an effective amount for the treatment of abnormal tissue of the male reproductive system such as prostate or testicular cancer, in combination or alternation with an effective amount of an androgen (such as testosterone) inhibitor including, but not limited to a selective androgen receptor modulator, a selective androgen receptor degrader, a complete androgen receptor degrader, or another form of partial or complete androgen antagonist. In certain embodiments, the prostate or testicular cancer is androgen-resistant.

Non-limiting examples of anti-androgen compounds are provided in WO 2011/156518 and U.S. Pat. Nos. 8,455,534 and 8,299,112. Additional non-limiting examples of anti-androgen compounds include: enzalutamide, apalutamide, cyproterone acetate, chlormadinone acetate, spironolactone, canrenone, drospirenone, ketoconazole, topilutamide, abiraterone acetate, and cimetidine.

In certain embodiments, the bioactive agent is an ALK inhibitor. Examples of ALK inhibitors include but are not limited to Crizotinib, Alectinib, ceritinib, TAE684 (NVP-TAE684), GSK1838705A, AZD3463, ASP3026, PF-06463922, entrectinib (RXDX-101), and AP26113.

In certain embodiments, the bioactive agent is an HER-2 inhibitor. Examples of HER-2 inhibitors include trastuzumab, lapatinib, ado-trastuzumab emtansine, and pertuzumab.

In certain embodiments, the bioactive agent is a CD20 inhibitor. Examples of CD20 inhibitors include obinutuzumab (GAZYVA®), rituximab (RITUXAN®), fatumumab, ibritumomab, tositumomab, and ocrelizumab.

In certain embodiments, the bioactive agent is a JAK3 inhibitor. Examples of JAK3 inhibitors include tasocitinib.

In certain embodiments, the bioactive agent is a BCL-2 inhibitor. Examples of BCL-2 inhibitors include venetoclax, ABT-199 (4-[4-[[2-(4-Chlorophenyl)-4,4-dimethylcyclohex-1-en-1-yl]methyl]piperazin-1-yl]-N-[[3-nitro-4-[[(tetrahydro-2H-pyran-4-yl)methyl]amino]phenyl]sulfonyl]-2-[(1H-pyrrolo[2,3-b]pyridin-5-yl)oxy]benzamide), ABT-737 (4-[4-[[2-(4-chlorophenyl)phenyl]methyl]piperazin-1-yl]-N-[4-[[(2R)-4-(dimethylamino)-1-phenylsulfanylbutan-2-yl]amino]-3-nitrophenyl]sulfonylbenzamide) (navitoclax), ABT-263 ((R)-4-(4-((4′-chloro-4,4-dimethyl-3,4,5,6-tetrahydro-[1,1′-biphenyl]-2-yl)methyl)piperazin-1-yl)-N-((4-((4-morpholino-1-(phenylthi o)butan-2-yl)amino)-3((trifluoromethyl)sulfonyl)phenyl)sulfonyl)benzamide), GX15-070 (obatoclax mesylate, (2Z)-2-[(5Z)-5-[(3,5-dimethyl-1H-pyrrol-2-yl)methylidene]-4-methoxypyrrol-2-ylidene]indole; methanesulfonic acid))), 2-methoxy-antimycin A3, YC137 (4-(4,9-dioxo-4,9-dihydronaphtho[2,3-d]thiazol-2-ylamino)-phenyl ester), pogosin, ethyl 2-amino-6-bromo-4-(1-cyano-2-ethoxy-2-oxoethyl)-4H-chromene-3-carboxylate, Nilotinib-d3, TW-37 (N-[4-[[2-(1,1-Dimethyl ethyl)phenyl]sulfonyl]phenyl]-2,3,4-trihydroxy-5-[[2-(1-methylethyl)phenyl]methyl]benzamide), Apogossypolone (ApoG2), HA14-1, AT101, sabutoclax, gambogic acid, or G3139 (oblimersen).

In certain embodiments, the bioactive agent is a kinase inhibitor. In certain embodiments, the kinase inhibitor is selected from a phosphoinositide 3-kinase (PI3K) inhibitor, a Bruton's tyrosine kinase (BTK) inhibitor, or a spleen tyrosine kinase (Syk) inhibitor, or a combination thereof.

Examples of PI3 kinase inhibitors include, but are not limited to, Wortmannin, demethoxyviridin, perifosine, idelalisib, pictilisib, palomid 529, ZSTK474, PWT33597, CUDC-907, and AEZS-136, duvelisib, GS-9820, BKM120, GDC-0032 (Taselisib) (2-[4[2-(2-Isopropyl-5-methyl-1,2,4-triazol-3-yl)-5,6-dihydroimidazo[1,2-d][1,4]benzoxazepin-9-yl]pyrazol-1-yl]-2-methylpropanamide), MLN-1117 ((2R)-1-Phenoxy-2-butanyl hydrogen (S)-methylphosphonate; or Methyl(oxo) {[(2R)-1-phenoxy-2-butanyl]oxy}phosphonium)), BYL-719 ((2S)—N1-[4-Methyl-5-[2-(2,2,2-trifluoro-1,1-dimethylethyl)-4-pyridinyl]-2-thiazolyl]-1,2-pyrrolidinedicarboxamide), GSK2126458 (2,4-Difluoro-N-{2-(methyloxy)-5-[4-(4-pyridazinyl)-6-quinolinyl]-3-pyridinyl}benzenesulfonamide) (omipalisib), TGX-221 ((±)-7-Methyl-2-(morpholin-4-yl)-9-(1-phenylaminoethyl)-pyrido[1,2-a]-pyrimidin-4-one), GSK2636771 (2-Methyl-1-(2-methyl-3-(trifluoromethyl)benzyl)-6-morpholino-1H-benzo[d]imidazole-4-carboxylic acid dihydrochloride), KIN-193 ((R)-2-((1-(7-methyl-2-morpholino-4-oxo-4H-pyrido[1,2-a]pyrimidin-9-yl)ethyl)amino)benzoic acid), TGR-1202/RP5264, GS-9820 ((S)-1-(4-((2-(2-aminopyrimidin-5-yl)-7-methyl-4-mohydroxypropan-1-one), GS-1101 (5-fluoro-3-phenyl-2-([S)]-1-[9H-purin-6-ylamino]-propyl)-3H-quinazolin-4-one), AMG-319, GSK-2269557, SAR245409 (N-(4-(N-(3-((3,5-dimethoxyphenyl)amino)quinoxalin-2-yl)sulfamoyl)phenyl)-3-methoxy-4 methylbenzamide), BAY80-6946 (2-amino-N-(7-methoxy-8-(3-morpholinopropoxy)-2,3-dihydroimidazo[1,2-c]quinaz), AS 252424 (5-[1-[5-(4-Fluoro-2-hydroxy-phenyl)-furan-2-yl]-meth-(Z)-ylidene]-thiazolidine-2,4-dione), CZ 24832 (5-(2-amino-8-fluoro-[1,2,4]triazolo[1,5-a]pyridin-6-yl)-N-tert-butylpyridine-3-sulfonamide), Buparlisib (5-[2,6-Di(4-morpholinyl)-4-pyrimidinyl]-4-(trifluoromethyl)-2-pyridinamine), GDC-0941 (2-(1H-Indazol-4-yl)-6-[[4-(methylsulfonyl)-1-piperazinyl]methyl]-4-(4-morpholinyl)thieno[3,2-d]pyrimidine), GDC-0980 ((S)-1-(4-((2-(2-aminopyrimidin-5-yl)-7-methyl-4-morpholinothieno[3,2-d]pyrimidin-6 yl)methyl)piperazin-1-yl)-2-hydroxypropan-1-one (also known as RG7422)), SF1126 ((8S,14S,17S)-14-(carboxymethyl)-8-(3-guanidinopropyl)-17-(hydroxymethyl)-3,6,9,12,15-pentaoxo-1-(4-(4-oxo-8-phenyl-4H-chromen-2-yl)morpholino-4-ium)-2-oxa-7,10,13,16-tetraazaoctadecan-18-oate), PF-05212384 (N-[4-[[4-(Dimethylamino)-1-piperidinyl]carbonyl]phenyl]-N′-[4-(4,6-di-4-morpholinyl-1,3,5-triazin-2-yl)phenyf]urea) (gedatolisib), LY3023414, BEZ235 (2-Methyl-2-{4-[3-methyl-2-oxo-8-(quinolin-3-yl)-2,3-dihydro-1H-imidazo[4,5-c]quinolin-1-yl]phenyl}propanenitrile) (dactolisib), XL-765 (N-(3-(N-(3-(3,5-dimethoxyphenylamino)quinoxalin-2-yl)sulfamoyl)phenyl)-3-methoxy-4-methylbenzamide), and GSK1059615 (5-[[4-(4-Pyridinyl)-6-quinolinyl]methylene]-2,4-thiazolidenedione), PX886 ([(3 aR,6E,9S,9aR,10R,11aS)-6-[[bis(prop-2-enyl)amino]methylidene]-5-hydroxy-9-(methoxymethyl)-9a,11a-dimethyl-1,4,7-trioxo-2,3,3a,9,10,11-hexahydroindeno[4,5h]isochromen-10-yl]acetate (also known as sonolisib)), LY294002, AZD8186, PF-4989216, pilaralisib, GNE-317, PI-3065, PI-103, NU7441 (KU-57788), HS 173, VS-5584 (SB2343), CZC24832, TG100-115, A66, YM201636, CAY10505, PIK-75, PIK-93, AS-605240, BGT226 (NVP-BGT226), AZD6482, voxtalisib, alpelisib, IC-87114, TGI100713, CH5132799, PKI-402, copanlisib (BAY 80-6946), XL 147, PIK-90, PIK-293, PIK-294, 3-MA (3-methyladenine), AS-252424, AS-604850, apitolisib (GDC-0980; RG7422).

Examples of BTK inhibitors include ibrutinib (also known as PCI-32765)(IMBRUVICA®) (1-[(3R)-3-[4-amino-3-(4-phenoxy-phenyl)pyrazolo[3,4-d]pyrimidin-1-yl]piperidin-1-yl]prop-2-en-1-one), dianilinopyrimidine-based inhibitors such as AVL-101 and AVL-291/292 (N-(3-((5-fluoro-2-((4-(2-methoxyethoxy)phenyl)amino)pyrimidin-4-yl)amino)phenyl)acrylamide) (Avila Therapeutics) (see US Patent Publication No 2011/0117073, incorporated herein in its entirety), dasatinib ([N-(2-chloro-6-methylphenyl)-2-(6-(4-(2-hydroxyethyl)piperazin-1-yl)-2-methylpyrimidin-4-ylamino)thiazole-5-carboxamide], LFM-A13 (alpha-cyano-beta-hydroxy-beta-methyl-N-(2,5-ibromophenyl) propenamide), GDC-0834 ([R—N-(3-(6-(4-(1,4-dimethyl-3-oxopiperazin-2-yl)phenylamino)-4-methyl-5-oxo-4,5-dihydropyrazin-2-yl)-2-methylphenyl)-4,5,6,7-tetrahydrobenzo[b]thiophene-2-carboxamide], CGI-560 4-(tert-butyl)-N-(3-(8-(phenylamino)imidazo[1,2-a]pyrazin-6-yl)phenyl)benzamide, CGI-1746 (4-(tert-butyl)-N-(2-methyl-3-(4-methyl-6-((4-(morpholine-4-carbonyl)phenyl)amino)-5-oxo-4,5-dihydropyrazin-2-yl)phenyl)benzamide), CNX-774 (4-(4-((4-((3-acrylamidophenyl)amino)-5-fluoropyrimidin-2-yl)amino)phenoxy)-N-methylpicolinamide), CTA056 (7-benzyl-1-(3-(piperidin-1-yl)propyl)-2-(4-(pyridin-4-yl)phenyl)-1H-imidazo[4,5-g]quinoxalin-6(5H)-one), GDC-0834 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), GDC-0837 ((R)—N-(3-(6-((4-(1,4-dimethyl-3-oxopiperazin-2-yl)phenyl)amino)-4-methyl-5-oxo-4,5-dihydropyrazin-2-yl)-2-methylphenyl)-4,5,6,7-tetrahydrobenzo[b]thiophene-2-carboxamide), HM-71224, ACP-196, ONO-4059 (Ono Pharmaceuticals), PRT062607 (4-((3-(2H-1,2,3-triazol-2-yl)phenyl)amino)-2-(((1R,2S)-2-aminocyclohexyl)amino)pyrimidine-5-carboxamide hydrochloride), QL-47 (1-(1-acryloylindolin-6-yl)-9-(1-methyl-1H-pyrazol-4-yl)benzo[h][1,6]naphthyridin-2(1H)-one), and RN486 (6-cyclopropyl-8-fluoro-2-(2-hydroxymethyl-3-{1-methyl-5-[5-(4-methyl-piperazin-1-yl)-pyridin-2-yl amino]-6-oxo-1,6-dihydro-pyridin-3-yl}-phenyl)-2H-isoquinolin-1-one), and other molecules capable of inhibiting BTK activity, for example those BTK inhibitors disclosed in Akinleye et ah, Journal of Hematology & Oncology, 2013, 6:59, the entirety of which is incorporated herein by reference.

Syk inhibitors include, but are not limited to, cerdulatinib (4-(cyclopropylamino)-2-((4-(4-(ethylsulfonyl)piperazin-1-yl)phenyl)amino)pyrimidine-5-carboxamide), entospletinib (6-(1H-indazol-6-yl)-N-(4-morpholinophenyl)imidazo[1,2-a]pyrazin-8-amine), fostamatinib ([6-({5-Fluoro-2-[(3,4,5-trimethoxyphenyl)amino]-4-pyrimidinyl}amino)-2,2-dimethyl-3-oxo-2,3-dihydro-4H-pyrido[3,2-b][1,4]oxazin-4-yl]methyl dihydrogen phosphate), fostamatinib disodium salt (sodium (6-((5-fluoro-2-((3,4,5-trimethoxyphenyl)amino)pyrimidin-4-yl)amino)-2,2-dimethyl-3-oxo-2H-pyrido[3,2-b][1,4]oxazin-4(3H)-yl)methyl phosphate), BAY 61-3606 (2-(7-(3,4-Dimethoxyphenyl)-imidazo[1,2-c]pyrimidin-5-ylamino)-nicotinamide HCl), R09021 (6-[(1R,2S)-2-Amino-cyclohexylamino]-4-(5,6-dimethyl-pyridin-2-ylamino)-pyridazine-3-carboxylic acid amide), imatinib (Gleevac; 4-[(4-methylpiperazin-1-yl)methyl]-N-(4-methyl-3-{[4-(pyridin-3-yl)pyrimidin-2-yl]amino}phenyl)benzamide), staurosporine, GSK143 (2-(((3R,4R)-3-aminotetrahydro-2H-pyran-4-yl)amino)-4-(p-tolylamino)pyrimidine-5-carboxamide), PP2 (1-(tert-butyl)-3-(4-chlorophenyl)-1H-pyrazolo[3,4-d]pyrimidin-4-amine), PRT-060318 (2-(((1R,2S)-2-aminocyclohexyl)amino)-4-(m-tolylamino)pyrimidine-5-carboxamide), PRT-062607 (4-((3-(2H-1,2,3-triazol-2-yl)phenyl)amino)-2-(((1R,2S)-2-aminocyclohexyl)amino)pyrimidine-5-carboxamide hydrochloride), R112 (3,3′4(5-fluoropyrimidine-2,4-diyl)bis(azanediyl))diphenol), R348 (3-Ethyl-4-methylpyridine), R406 (6-((5-fluoro-2-(3,4,5-trimethoxyphenyl)amino)pyrimidin-4-yl)amino)-2,2-dimethyl-2H-pyrido[3,2-b][1,4]oxazin-3(4H)-one), piceatannol (3-Hydroxyresveratol), YM193306 (see Singh et al. Discovery and Development of Spleen Tyrosine Kinase (SYK) Inhibitors, J. Med. Chem. 2012, 55, 3614-3643), 7-azaindole, piceatannol, ER-27319 (see Singh et al. Discovery and Development of Spleen Tyrosine Kinase (SYK) Inhibitors, J. Med. Chem. 2012, 55, 3614-3643 incorporated in its entirety herein), Compound D (see Singh et al. Discovery and Development of Spleen Tyrosine Kinase (SYK) Inhibitors, J. Med. Chem. 2012, 55, 3614-3643 incorporated in its entirety herein), PRT060318 (see Singh et al. Discovery and Development of Spleen Tyrosine Kinase (SYK) Inhibitors, J. Med. Chem. 2012, 55, 3614-3643 incorporated in its entirety herein), luteolin (see Singh et al. Discovery and Development of Spleen Tyrosine Kinase (SYK) Inhibitors, J. Med. Chem. 2012, 55, 3614-3643 incorporated in its entirety herein), apigenin (see Singh et al.

Discovery and Development of Spleen Tyrosine Kinase (SYK) Inhibitors, J. Med. Chem. 2012, 55, 3614-3643 incorporated in its entirety herein), quercetin (see Singh et al. Discovery and Development of Spleen Tyrosine Kinase (SYK) Inhibitors, J. Med. Chem. 2012, 55, 3614-3643 incorporated in its entirety herein), fisetin (see Singh et al. Discovery and Development of Spleen Tyrosine Kinase (SYK) Inhibitors, J. Med. Chem. 2012, 55, 3614-3643 incorporated in its entirety herein), myricetin (see Singh et al. Discovery and Development of Spleen Tyrosine Kinase (SYK) Inhibitors, J. Med. Chem. 2012, 55, 3614-3643 incorporated in its entirety herein), morin (see Singh et al. Discovery and Development of Spleen Tyrosine Kinase (SYK) Inhibitors, J. Med. Chem. 2012, 55, 3614-3643 incorporated in its entirety herein).

In certain embodiments, the bioactive agent is a MEK inhibitor. MEK inhibitors are well known, and include, for example, trametinib/GSK1120212 (N-(3-{3-Cyclopropyl-5-[(2-fluoro-4-iodophenyl)amino]-6,8-dimethyl-2,4,7-trioxo-3,4,6,7-tetrahydropyrido[4,3-d]pyrimidin-1(2H-yl}phenyl)acetamide), selumetinib (6-(4-bromo-2-chloroanilino)-7-fluoro-N-(2-hydroxyethoxy)-3-methylbenzimidazole-5-carboxamide), pimasertib/AS703026NISC 1935369 ((S)—N-(2,3-dihydroxypropyl)-3-((2-fluoro-4-iodophenyl)amino)isonicotinamide), XL-518/GDC-0973 (1-({3,4-difluoro-2-[(2-fluoro-4-iodophenyl)amino]phenyl}carbonyl)-3-[(2S)-piperidin-2-yl]azetidin-3-ol), refametinib/BAY869766/RDEA1 19 (N-(3,4-difluoro-2-(2-fluoro-4-iodophenyl amino)-6-methoxyphenyl)-1-(2,3-dihydroxypropyl)cyclopropane-1-sulfonamide), PD-0325901 (N-[(2R)-2,3-Dihydroxypropoxy]-3,4-difluoro-2-[(2-fluoro-4-iodophenyl)amino]-benzamide), TAK733 ((R)-3-(2,3-Dihydroxypropyl)-6-fluoro-5-(2-fluoro-4-iodophenylamino)-8-methylpyrido[2,3-d]pyrimidine-4,7(3H,8H)-dione), MEK162/ARRY438162 (5-[(4-Bromo-2-fluorophenyl)amino]-4-fluoro-N-(2-hydroxyethoxy)-1-methyl-1H-benzimidazole-6-carboxamide), R05126766 (3-[[3-Fluoro-2-(methyl sulfamoyl amino)-4-pyridyl]methyl]-4-methyl-7-pyrimidin-2-yloxychromen-2-one), WX-554, R04987655/CH4987655 (3,4-difluoro-2-((2-fluoro-4-iodophenyl)amino)-N-(2-hydroxyethoxy)-5-oxo-1,2-oxazinan-2yl)methyl)benzamide), or AZD8330 (2-((2-fluoro-4-iodophenyl)amino)-N-(2 hydroxyethoxy)-1,5-dimethyl-6-oxo-1,6-dihydropyridine-3-carboxamide), U0126-EtOH, PD184352 (CI-1040), GDC-0623, BI-847325, cobimetinib, PD98059, BIX02189, BIX02188, binimetinib, SL-327, TAK-733, PD318088.

In certain embodiments, the bioactive agent is a Raf inhibitor. Raf inhibitors are known and include, for example, Vemurafinib (N-[3-[[5-(4-Chlorophenyl)-1H-pyrrolo[2,3-b]pyridin-3-yl]carbonyl]-2,4-difluorophenyl]-1-propanesulfonamide), sorafenib tosylate (4-[4-[[4-chloro-3-(trifluoromethyl)phenyl]carbamoylamino]phenoxy]-N-methylpyridine-2-carboxamide; 4-methylbenzenesulfonate), AZ628 (3-(2-cyanopropan-2-yl)-N-(4-methyl-3-(3-methyl-4-oxo-3,4-dihydroquinazolin-6-ylamino)phenyl)benzamide), NVP-BHG712 (4-methyl-3-(1-methyl-6-(pyridin-3-yl)-1H-pyrazolo[3,4-d]pyrimidin-4-ylamino)-N-(3-(trifluoromethyl)phenyl)benzamide), RAF-265 (1-methyl-5-[2-[5-(trifluoromethyl)-1H-imidazol-2-yl]pyridin-4-yl]oxy-N-[4-(trifluoromethyl)phenyl]benzimidazol-2-amine), 2-Bromoaldisine (2-Bromo-6,7-dihydro-1H,5H-pyrrolo[2,3-c]azepine-4,8-dione), Raf Kinase Inhibitor IV (2-chloro-5-(2-phenyl-5-(pyridin-4-yl)-1H-imidazol-4-yl)phenol), Sorafenib N-Oxide (4-[4-[[[[4-Chloro-3(trifluoroMethyl)phenyl]aMino]carbonyl]aMino]phenoxy]-N-Methyl-2pyridinecarboxaMide 1-Oxide), PLX-4720, dabrafenib (GSK2118436), GDC-0879, RAF265, AZ 628, SB590885, ZM336372, GW5074, TAK-632, CEP-32496, LY3009120, and GX818 (encorafenib (BRAFTOVI®)).

In certain embodiments, the bioactive agent is an EGFR inhibitor, including, for example gefitinib (IRESSA®), lapatinib (TYKERB®), osimertinib (TAGRISSO®), neratinib (NERLYNX®), vandetanib (CAPRELSA®), dacomitinib (VIZIMPRO®), rociletinib (XEGAFRI™), afatinib (GLOTRIF®), GIOTRIFF™, AFANIX™), lazertinib, or nazartib.

Additional examples of EGFR inhibitors include rociletinib (CO-1686), olmutinib (Olita), naquotinib (ASP8273), nazartinib (EGF816), PF-06747775, icotinib (BPI-2009), neratinib (HKI-272; PB272); avitinib (AC0010), EA1045, tarloxotinib (TH-4000; PR-610), PF-06459988 (Pfizer), tesevatinib (XL647; EXEL-7647; KD-019), transtinib, WZ-3146, WZ8040, CNX-2006, dacomitinib (PF-00299804; Pfizer), brigatinib (Alunbrig), lorlatinib, and PF-06747775 (PF7775).

In certain embodiments, the bioactive agent is a first-generation EGFR inhibitor such as erlotinib, gefitinib, or lapatinib. In certain embodiments, the bioactive agent is a second-generation EGFR inhibitor such as afatinib and/or dacomitinib. In certain embodiments, the bioactive agent is a third-generation EGFR inhibitor such as osimertinib.

In certain aspects Compound 1 is administered in combination with a ATP-site binding inhibitor of EGFR or mutant EGFR. Non-limiting examples of ATP-site binding inhibitors of EGFR include osimertinib, naquotinib, mavelertinib, spebrutinib, and AZ5104.

In certain embodiments a compound described herein is administered to a patient in need thereof in combination with osimertinib.

In certain embodiments a compound described herein is administered to a patient in need thereof in combination with naquotinib.

In certain embodiments a compound described herein is administered to a patient in need thereof in combination with mavelertinib.

In certain embodiments a compound described herein is administered to a patient in need thereof in combination with spebrutinib.

In certain embodiments a compound described herein is administered to a patient in need thereof in combination with AZ5104.

In certain embodiments a compound described herein is administered to a patient in need thereof in combination with rociletinib.

In certain embodiments a compound described herein is administered to a patient in need thereof in combination with avitinib.

In certain embodiments a compound described herein is administered to a patient in need thereof in combination with lazertinib.

In certain embodiments a compound described herein is administered to a patient in need thereof in combination with nazartinib.

In certain embodiments a compound described herein is administered to a patient in need thereof in combination with an EGFR antibody, for example, cetuximab, panitumab, or necitumab.

In certain embodiments a compound described herein is administered to a patient in need thereof in combination with cetuximab.

In certain embodiments a compound described herein is administered to a patient in need thereof in combination with panitumab.

In certain embodiments a compound described herein is administered to a patient in need thereof in combination with necitumab.

In certain embodiments, the bioactive agent is a c-MET inhibitor, for example, crizotinib (Xalkori, Crizonix), tepotinib (XL880, EXEL-2880, GSK1363089, GSK089), or tivantinib (ARQ197).

In certain embodiments, the bioactive agent is an AKT inhibitor, including, but not limited to, MK-2206, GSK690693, perifosine, (KRX-0401), GDC-0068, triciribine, AZD5363, honokiol, PF-04691502, and miltefosine, a FLT-3 inhibitor, including, but not limited to, P406, dovitinib, quizartinib (AC220), amuvatinib (MP-470), tandutinib (MLN518), ENMD-2076, and KW-2449, or a combination thereof.

In certain embodiments, the bioactive agent is an mTOR inhibitor. Examples of mTOR inhibitors include, but are not limited to, rapamycin and its analogs, everolimus (Afinitor), temsirolimus, ridaforolimus, sirolimus, and deforolimus.

In certain embodiments, the bioactive agent is a RAS inhibitor. Examples of RAS inhibitors include but are not limited to Reolysin and siG12D LODER.

In certain embodiments, the bioactive agent is a HSP inhibitor. HSP inhibitors include but are not limited to Geldanamycin or 17-N-Allylamino-17-demethoxygeldanamycin (17AAG), and Radicicol.

Additional bioactive compounds include, for example, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an aurora kinase inhibitor, a PIK-1 modulator, an HDAC inhibitor, a c-MET inhibitor, a PARP inhibitor, a Cdk inhibitor, an IGFR-TK inhibitor, an anti-HGF antibody, a focal adhesion kinase inhibitor, a Map kinase kinase (mek) inhibitor, a VEGF trap antibody, pemetrexed, panitumumab, amrubicin, oregovomab, Lep-etu, nolatrexed, azd2171, batabulin, atumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258); 3-[5-(methylsulfonylpiperadinemethyl)-indolyl-quinolone, vatalanib, AG-013736, AVE-0005, goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, arnsacrine, anagrelide, L-asparaginase, Bacillus Calmette-Guerin (BCG) vaccine, adriamycin, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gleevec, gemcitabine, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonist, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

In certain embodiments the compound is administered in combination with ifosfamide.

In certain embodiments, the bioactive agent is selected from, but are not limited to, Imatinib mesylate (Gleevac®), Dasatinib (Sprycel®), Nilotinib (Tasigna®), Bosutinib (Bosulif®), Trastuzumab (Herceptin®), trastuzumab-DM1, Pertuzumab (Perjeta™), Lapatinib (Tykerb®), Gefitinib (Iressa®), Erlotinib (Tarceva®), Cetuximab (Erbitux®), Panitumumab (Vectibix®), Vandetanib (Caprelsa®), Vemurafenib (Zelboraf®), Vorinostat (Zolinza®), Romidepsin (Istodax®), Bexarotene (Tagretin®), Alitretinoin (Panretin®), Tretinoin (Vesanoid®), Carfilizomib (Kyprolis™), Pralatrexate (Folotyn®), Bevacizumab (Avastin®), Ziv-aflibercept (Zaltrap®), Sorafenib (Nexavar®), Sunitinib (Sutent®), Pazopanib (Votrient®), Regorafenib (Stivarga®), and Cabozantinib (Cometriq™).

In certain aspects, the bioactive agent is an anti-inflammatory agent, a chemotherapeutic agent, a radiotherapeutic, an additional therapeutic agent, or an immunosuppressive agent.

Suitable chemotherapeutic bioactive agents include, but are not limited to, a radioactive molecule, a toxin, also referred to as cytotoxin or cytotoxic agent, which includes any agent that is detrimental to the viability of cells, and liposomes or other vesicles containing chemotherapeutic compounds. General anticancer pharmaceutical agents include: Vincristine (Oncovin®) or liposomal vincristine (Margibo®), Daunorubicin (daunomycin or Cerubidine®) or doxorubicin (Adriamycin®), Cytarabine (cytosine arabinoside, ara-C, or Cytosar®), L-asparaginase (Elspar®) or PEG-L-asparaginase (pegaspargase or Oncaspar®), Etoposide (VP-16), Teniposide (Vumon®), 6-mercaptopurine (6-MP or Purinethol®), Methotrexate, Cyclophosphamide (Cytoxan®), Prednisone, Dexamethasone (Decadron), imatinib (Gleevec®), dasatinib (Sprycel®), nilotinib (Tasigna®), bosutinib (Bosulif®), and ponatinib (Iclusig™)

Examples of additional suitable chemotherapeutic agents include, but are not limited to 1-dehydrotestosterone, 5-fluorouracil decarbazine, 6-mercaptopurine, 6-thioguanine, actinomycin D, adriamycin, aldesleukin, an alkylating agent, allopurinol sodium, altretamine, amifostine, anastrozole, anthramycin (AMC)), an anti-mitotic agent, cis-dichlorodiamine platinum (II) (DDP) cisplatin), diamino dichloro platinum, anthracycline, an antibiotic, an antimetabolite, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

In some embodiments, the compound described herein is administered in combination with a chemotherapeutic agent (e.g., a cytotoxic agent or other chemical compound useful in the treatment of cancer). Examples of chemotherapeutic agents include alkylating agents, antimetabolites, folic acid analogs, pyrimidine analogs, purine analogs and related inhibitors, vinca alkaloids, epipodopyyllotoxins, antibiotics, L-Asparaginase, topoisomerase inhibitors, interferons, platinum coordination complexes, anthracenedione substituted urea, methyl hydrazine derivatives, adrenocortical suppressant, adrenocorticosteroides, progestins, estrogens, antiestrogen, androgens, antiandrogen, and gonadotropin-releasing hormone analog. Also included is 5-fluorouracil (5-FU), leucovorin (LV), irenotecan, oxaliplatin, capecitabine, paclitaxel, and doxetaxel. Non-limiting examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Inti. Ed Engl. 33:183-186 (1994)); dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin, including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® (paclitaxel; Bristol-Myers Squibb Oncology, Princeton, NJ), ABRAXANE®, cremophor-free, albumin-engineered nanoparticle formulation of paclitaxel (American Pharmaceutical Partners, Schaumberg, IL), and TAXOTERE® doxetaxel (Rhone-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE®, vinorelbine; novantrone; teniposide; edatrexate; daunomycin; aminopterin; xeloda; ibandronate; irinotecan (e.g., CPT-1 1); topoisomerase inhibitor RFS 2000; difluoromethylornithine (DMFO); retinoids such as retinoic acid; capecitabine; and pharmaceutically acceptable salts, acids or derivatives of any of the above. Two or more chemotherapeutic agents can be used in a cocktail to be administered in combination with the compound described herein. Suitable dosing regimens of combination chemotherapies are known in the ar. For example combination dosing regimes are described in Saltz et al., Proc. Am. Soc. Clin. Oncol. 18:233a (1999) and Douillard et al., Lancet 355(9209): 1041-1047 (2000).

Additional therapeutic agents that can be administered in combination with a Compound disclosed herein can include bevacizumab, sutinib, sorafenib, 2-methoxyestradiol or 2ME2, finasunate, vatalanib, vandetanib, aflibercept, volociximab, etaracizumab (MEDI-522), cilengitide, cetuximab, panitumumab, gefitinib, trastuzumab, dovitinib, figitumumab, atacicept, rituximab, alemtuzumab, aldesleukine, atlizumab, tocilizumab, temsirolimus, everolimus, lucatumumab, dacetuzumab, HLL1, huN901-DM1, atiprimod, natalizumab, bortezomib, carfilzomib, marizomib, tanespimycin, saquinavir mesylate, ritonavir, nelfinavir mesylate, indinavir sulfate, belinostat, panobinostat, mapatumumab, lexatumumab, dulanermin, ABT-737, oblimersen, plitidepsin, talmapimod, P276-00, enzastaurin, tipifarnib, perifosine, imatinib, dasatinib, lenalidomide, thalidomide, simvastatin, celecoxib, bazedoxifene, AZD4547, rilotumumab, oxaliplatin (Eloxatin), PD0332991, ribociclib (LEE011), amebaciclib (LY2835219), HDM201, fulvestrant (Faslodex), exemestane (Aromasin), PIM447, ruxolitinib (INC424), BGJ398, necitumumab, pemetrexed (Alimta), and ramucirumab (IMC-1121B).

In certain embodiments, the additional therapy is a monoclonal antibody (MAb). Some MAbs stimulate an immune response that destroys cancer cells. Similar to the antibodies produced naturally by B cells, these MAbs may “coat” the cancer cell surface, triggering its destruction by the immune system. For example, bevacizumab targets vascular endothelial growth factor (VEGF), a protein secreted by tumor cells and other cells in the tumor's microenvironment that promotes the development of tumor blood vessels. When bound to bevacizumab, VEGF cannot interact with its cellular receptor, preventing the signaling that leads to the growth of new blood vessels. MAbs that bind to cell surface growth factor receptors prevent the targeted receptors from sending their normal growth-promoting signals. They may also trigger apoptosis and activate the immune system to destroy tumor cells.

In one aspect of the present invention, the bioactive agent is an immunosuppressive agent. The immunosuppressive agent can be a calcineurin inhibitor, e.g. a cyclosporin or an ascomycin, e.g. Cyclosporin A (NEORAL®), FK506 (tacrolimus), pimecrolimus, a mTOR inhibitor, e.g. rapamycin or a derivative thereof, e.g. Sirolimus (RAPAMUNE®), Everolimus (Certican®), temsirolimus, zotarolimus, biolimus-7, biolimus-9, a rapalog, e.g. ridaforolimus, azathioprine, campath 1H, a S1P receptor modulator, e.g. fingolimod or an analogue thereof, an anti IL-8 antibody, mycophenolic acid or a salt thereof, e.g. sodium salt, or a prodrug thereof, e.g. Mycophenolate Mofetil (CELLCEPT®), OKT3 (ORTHOCLONE OKT3®), Prednisone, ATGAM®, THYMOGLOBULIN®, Brequinar Sodium, OKT4, T10B9.A-3A, 33B3.1, 15-deoxyspergualin, tresperimus, Leflunomide ARAVA®, CTLAI-Ig, anti-CD25, anti-IL2R, Basiliximab (SIIVIULECT®), Daclizumab (ZENAPAX®), mizorbine, methotrexate, dexamethasone, ISAtx-247, SDZ ASM 981 (pimecrolimus, Elidel®), CTLA41g (Abatacept), belatacept, LFA31g, etanercept (sold as Enbrel® by Immunex), adalimumab (Humira®), infliximab (Remicade®), an anti-LFA-1 antibody, natalizumab (Antegren®), Enlimomab, gavilimomab, antithymocyte immunoglobulin, siplizumab, Alefacept efalizumab, pentasa, mesalazine, asacol, codeine phosphate, benorylate, fenbufen, naprosyn, diclofenac, etodolac and indomethacin, aspirin and ibuprofen.

In some embodiments, the bioactive agent is a therapeutic agent which is a biologic such a cytokine (e.g., interferon or an interleukin (e.g., IL-2)) used in cancer treatment. In some embodiments the biologic is an anti-angiogenic agent, such as an anti-VEGF agent, e.g., bevacizumab (AVASTIN®). In some embodiments the biologic is an immunoglobulin-based biologic, e.g., a monoclonal antibody (e.g., a humanized antibody, a fully human antibody, an Fc fusion protein or a functional fragment thereof) that agonizes a target to stimulate an anti-cancer response, or antagonizes an antigen important for cancer. Such agents include RITUXAN® (rituximab); ZENAPAX® (daclizumab); SIMULECT® (basiliximab); SYNAGIS® (palivizumab); REMICADE® (infliximab); HERCEPTIN® (trastuzumab); MYLOTARG® (gemtuzumab ozogamicin); CAMPATH® (alemtuzumab); ZEVALIN® (ibritumomab tiuxetan); HUMIRA® (adalimumab); XOLAIR® (omalizumab); BEXXAR® (tositumomab-1-131); RAPTIVA® (efalizumab); ERBITUX® (cetuximab); AVASTIN® (bevacizumab); TYSABRI® (natalizumab); ACTEMRA® (tocilizumab); VECTIBIX® (panitumumab); LUCENTIS® (ranibizumab); SOURIS® (eculizumab); CIMZIA® (certolizumab pegol); S ONI (golimumab); HARTS® (canakinumab); STELARA® (ustekinumab); ARZERRA® (ofatumumab); PROLIA® (denosumab); NUMAX® (motavizumab); ABTHRAX® (raxibacumab); BENLYSTA® (belimumab); YERVOY® (ipilimumab); ADCETRIS® (brentuximab vedotin); PERJETA® (pertuzumab); KADCYLA® (ado-trastuzumab emtansine); and GAZYVA® (obinutuzumab). Also included are antibody-drug conjugates.

The combination therapy may include a therapeutic agent which is a non-drug treatment. For example, the compound could be administered in addition to radiation therapy, cryotherapy, hyperthermia, and/or surgical excision of tumor tissue.

Compounds administered “in combination” as the term is used herein can refer to simultaneous administration or administration of the two compounds at different times or on different days in the treatment cycle.

In certain embodiments the first and second therapeutic agents are administered simultaneously or sequentially, in either order. The first therapeutic agent may be administered immediately, up to 1 hour, up to 2 hours, up to 3 hours, up to 4 hours, up to 5 hours, up to 6 hours, up to 7 hours, up to, 8 hours, up to 9 hours, up to 10 hours, up to 11 hours, up to 12 hours, up to 13 hours, 14 hours, up to hours 16, up to 17 hours, up 18 hours, up to 19 hours up to 20 hours, up to 21 hours, up to 22 hours, up to 23 hours up to 24 hours or up to 1-7, 1-14, 1-21 or 1-30 days before or after the second therapeutic agent.

In certain embodiments the second therapeutic agent is administered on a different dosage schedule than the compound described herein. For example the second therapeutic agent may have a treatment holiday of 1 day, 2 days, 3 days, 4 days, 5 days, 6 days, 7 days, 8 days, 9 days, 10 days, 11 days, 12 days, 13 days, or 14 days per treatment cycle. In another embodiment the first therapeutic agent has a treatment holiday. For example the first therapeutic agent may have a treatment holiday of 1 day, 2 days, 3 days, 4 days, 5 days, 6 days, 7 days, 8 days, 9 days, 10 days, 11 days, 12 days, 13 days, or 14 days per treatment cycle. In certain embodiments both the first and second therapeutic have a treatment holiday.

### VII. Pharmaceutical Compositions

A compound of Formula I, II, III, or IV or a pharmaceutically acceptable salt thereof can be used as a therapeutically active substance, e.g. in the form of a pharmaceutical preparations. The pharmaceutical preparations can be administered orally, e.g. in the form of tablets, coated tablets, dragées, hard and soft gelatin capsules, solutions, emulsions or suspensions. In other embodiments the compound is administered paternally, for example by intravenous administration. The administration can, however, also be effected rectally, e.g. in the form of suppositories, or parenterally, e.g. in the form of injection solutions.

The compounds of Formula I, II, III, or IV and the pharmaceutically acceptable salts thereof can be processed with pharmaceutically inert, inorganic or organic carriers for the production of pharmaceutical preparations. Lactose, corn starch or derivatives thereof, talc, stearic acids or its salts and the like can be used, for example, as such carriers for tablets, coated tablets, dragées and hard gelatin capsules. Suitable carriers for soft gelatin capsules are, for example, vegetable oils, waxes, fats, semi-solid and liquid polyols and the like. Depending on the nature of the active substance no carriers are however usually required in the case of soft gelatin capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical preparations can, moreover, contain pharmaceutically acceptable auxiliary substances such as preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

Medicaments containing a compound of Formula I, II, III, or IV or a pharmaceutically acceptable salt thereof and a therapeutically inert carrier are also provided by the present invention, as is a process for their production, which comprises bringing one or more compounds of Formula I, II, III, or IV and/or pharmaceutically acceptable salts thereof and, if desired, one or more other therapeutically valuable substances into a galenical administration form together with one or more therapeutically inert carriers.

The dosage can vary within wide limits and will, of course, have to be adjusted to the individual requirements in each particular case. In the case of oral administration the dosage for adults can vary from about 0.01 mg to about 1000 mg per day of a compound of general Formula I, II, III, or IV or of the corresponding amount of a pharmaceutically acceptable salt thereof. The daily dosage may be administered as single dose or in divided doses and, in addition, the upper limit can also be exceeded when this is found to be indicated.

The following examples illustrate the present invention without limiting it, but serve merely as representative thereof. The pharmaceutical preparations conveniently contain about 1-500 mg, particularly 1-100 mg, of a compound of Formula I, II, III, or IV. Examples of compositions according to the invention are: In certain embodiments the pharmaceutical composition is in a dosage form that contains from about 0.1 mg to about 2000 mg, from about 10 mg to about 1000 mg, from about 100 mg to about 800 mg, or from about 200 mg to about 600 mg of the active compound and optionally from about 0.1 mg to about 2000 mg, from about 10 mg to about 1000 mg, from about 100 mg to about 800 mg, or from about 200 mg to about 600 mg of an additional active agent in a unit dosage form. Examples are dosage forms with at least 0.1, 1, 5, 10, 25, 50, 100, 200, 250, 300, 400, 500, 600, 700, or 750 mg of active compound, or its salt.

In some embodiments, compounds disclosed herein or used as described are administered once a day (QD), twice a day (BID), or three times a day (TID). In some embodiments, compounds disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the compound described herein is administered once a day, twice a day, three times a day, or four times a day.

In certain embodiments the compound described herein is administered orally once a day. In certain embodiments the compound described herein is administered orally twice a day. In certain embodiments the compound described herein is administered orally three times a day. In certain embodiments the compound described herein is administered orally four times a day.

In certain embodiments the compound described herein is administered intravenously once a day. In certain embodiments the compound described herein is administered intravenously twice a day. In certain embodiments the compound described herein is administered intravenously three times a day. In certain embodiments the compound described herein is administered intravenously four times a day.

In some embodiments the compound described herein is administered with a treatment holiday in between treatment cycles. For example the compound may have a treatment holiday of 1 day, 2 days, 3 days, 4 days, 5 days, 6 days, 7 days, 8 days, 9 days, 10 days, 11 days, 12 days, 13 days, or 14 days per treatment cycle.

In some embodiments a loading dose is administered to begin treatment. For example, the compound may be administered about 1.5×, about 2×, about 2.5×, about 3×, about 3.5×, about 4×, about 4.5×, about 5×, about 5.5×, about 6×, about 6.5×, about 7×, about 7.5×, about 8×, about 8.5×, about 9×, about 9.5×, or about 10× higher dose on the first day of treatment than the remaining days of treatment in the treatment cycle. Additional exemplary loading doses include about 1.5×, about 2×, about 2.5×, about 3×, about 3.5×, about 4×, about 4.5×, about 5×, about 5.5×, about 6×, about 6.5×, about 7×, about 7.5×, about 8×, about 8.5×, about 9×, about 9.5×, or about 10× higher dose on the first 2, 3, 4, 5, 6, 7, 8, 9, or 10 days of treatment than the remaining days of treatment in the treatment cycle.

The pharmaceutical composition may also include a molar ratio of the active compound and an additional active agent. For example the pharmaceutical composition may contain a molar ratio of about 0.5:1, about 1:1, about 2:1, about 3:1 or from about 1.5:1 to about 4:1 of an anti-inflammatory or immunosuppressing agent.

These compositions can contain any amount of active compound that achieves the desired result, for example between 0.1 and 99 weight % (wt. %) of the compound and usually at least about 5 wt. % of the compound. Some embodiments contain from about 25 wt. % to about 50 wt. or from about 5 wt. % to about 75 wt. % of the compound.

A pharmaceutically or therapeutically effective amount of the composition will be delivered to the patient. The precise effective amount will vary from patient to patient, and will depend upon the species, age, the subject's size and health, the nature and extent of the condition being treated, recommendations of the treating physician, and the therapeutics or combination of therapeutics selected for administration. The effective amount for a given situation can be determined by routine experimentation. For purposes of the disclosure, a therapeutic amount may for example be in the range of about 0.01 mg/kg to about 250 mg/kg body weight, more typically about 0.1 mg/kg to about 10 mg/kg, in at least one dose. The subject can be administered as many doses as is required to reduce and/or alleviate the signs, symptoms, or causes of the disorder in question, or bring about any other desired alteration of a biological system. When desired, formulations can be prepared with enteric coatings adapted for sustained or controlled release administration of the active ingredient.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packed tablets, capsules, and powders in vials or ampoules. Also, the unit dosage form can be a capsule, tablet, cachet, or lozenge itself, or it can be the appropriate number of any of these in packaged form.

In certain embodiments the compound is administered as a pharmaceutically acceptable salt. Non-limiting examples of pharmaceutically acceptable salts include: acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, and valerate salts. Representative alkali or alkaline earth metal salts include sodium, lithium, potassium, calcium, and magnesium, as well as nontoxic ammonium, quaternary ammonium, and amine cations, including, but not limited to ammonium, tetramethylammonium, tetraethylammonium, methylamine, dimethylamine, trimethylamine, triethylamine, and ethylamine.

Thus, the composition of the disclosure can be administered as a pharmaceutical formulation including one suitable for oral (including buccal and sub-lingual), rectal, nasal, topical, transdermal, pulmonary, vaginal or parenteral (including intramuscular, intra-arterial, intrathecal, subcutaneous and intravenous), injections, inhalation or spray, intra-aortal, intracranial, subdermal, intraperitioneal, subcutaneous, or by other means of administration containing conventional pharmaceutically acceptable carriers. A typical manner of administration is oral, topical or intravenous, using a convenient daily dosage regimen which can be adjusted according to the degree of affliction.

Depending on the intended mode of administration, the pharmaceutical compositions can be in the form of solid, semi-solid or liquid dosage forms, such as, for example, tablets, suppositories, pills, capsules, powders, liquids, syrup, suspensions, creams, ointments, lotions, paste, gel, spray, aerosol, foam, or oil, injection or infusion solution, a transdermal patch, a subcutaneous patch, an inhalation formulation, in a medical device, suppository, buccal, or sublingual formulation, parenteral formulation, or an ophthalmic solution, or the like, preferably in unit dosage form suitable for single administration of a precise dosage.

Some dosage forms, such as tablets and capsules, are subdivided into suitably sized unit doses containing appropriate quantities of the active components, e.g., an effective amount to achieve the desired purpose. The compositions will include an effective amount of the selected drug in combination with a pharmaceutically acceptable carrier and, in addition, can include other pharmaceutical agents, adjuvants, diluents, buffers, and the like.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to adjuvants, binders, buffering agents, coloring agents, diluents, disintegrants, excipients, emulsifiers, flavorants, gels, glidents, lubricants, preservatives, stabilizers, surfactants, solubilizer, tableting agents, wetting agents or solidifying material.

Some carriers may be listed in more than one class, for example vegetable oil may be used as a lubricant in some formulations and a diluent in others.

Exemplary pharmaceutically acceptable carriers include sugars, starches, celluloses, powdered tragacanth, malt, gelatin; talc, petroleum jelly, lanoline, polyethylene glycols, alcohols, transdermal enhancers and vegetable oils. Optional active agents may be included in a pharmaceutical composition, which do not substantially interfere with the activity of the compound described herein.

Some excipients include, but are not limited, to liquids such as water, saline, glycerol, polyethylene glycol, hyaluronic acid, ethanol, and the like. The compound can be provided, for example, in the form of a solid, a liquid, spray dried material, a microparticle, nanoparticle, controlled release system, etc., as desired according to the goal of the therapy. Suitable excipients for non-liquid formulations are also known to those of skill in the art. A thorough discussion of pharmaceutically acceptable excipients and salts is available in Remington's Pharmaceutical Sciences, 18th Edition (Easton, Pennsylvania: Mack Publishing Company, 1990).

Additionally, auxiliary substances, such as wetting or emulsifying agents, biological buffering substances, surfactants, and the like, can be present in such vehicles. A biological buffer can be any solution which is pharmacologically acceptable, and which provides the formulation with the desired pH, i.e., a pH in the physiologically acceptable range. Examples of buffer solutions include saline, phosphate buffered saline, Tris buffered saline, Hank's buffered saline, and the like.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmaceutically administrable compositions can, for example, be prepared by dissolving, dispersing, and the like, an active compound as described herein and optional pharmaceutical adjuvants in an excipient, such as, for example, water, saline, aqueous dextrose, glycerol, ethanol, and the like, to thereby form a solution or suspension. If desired, the pharmaceutical composition to be administered can also contain minor amounts of nontoxic auxiliary substances such as wetting or emulsifying agents, pH buffering agents and the like, for example, sodium acetate, sorbitan monolaurate, triethanolamine sodium acetate, triethanolamine oleate, and the like. Actual methods of preparing such dosage forms are known, or will be apparent, to those skilled in this art; for example, see Remington's Pharmaceutical Sciences, referenced above.

In yet another embodiment provided is the use of permeation enhancer excipients including polymers such as: polycations (chitosan and its quaternary ammonium derivatives, poly-L-arginine, aminated gelatin); polyanions (N-carboxymethyl chitosan, poly-acrylic acid); and, thiolated polymers (carboxymethyl cellulose-cysteine, polycarbophil-cysteine, chitosan-thiobutylamidine, chitosan-thioglycolic acid, chitosan-glutathione conjugates).

In certain embodiments the excipient is selected from butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

The pharmaceutical compositions/combinations can be formulated for oral administration. For oral administration, the composition will generally take the form of a tablet, capsule, a softgel capsule or can be an aqueous or nonaqueous solution, suspension or syrup. Tablets and capsules are typical oral administration forms. Tablets and capsules for oral use can include one or more commonly used carriers such as lactose and corn starch. Lubricating agents, such as magnesium stearate, are also typically added. Typically, the compositions of the disclosure can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

When liquid suspensions are used, the active agent can be combined with any oral, non-toxic, pharmaceutically acceptable inert carrier such as ethanol, glycerol, water, and the like and with emulsifying and suspending agents. If desired, flavoring, coloring and/or sweetening agents can be added as well. Other optional components for incorporation into an oral formulation herein include, but are not limited to, preservatives, suspending agents, thickening agents, and the like.

For ocular delivery, the compound can be administered, as desired, for example, via intravitreal, intrastromal, intracameral, sub-tenon, sub-retinal, retro-bulbar, peribulbar, suprachorodial, conjunctival, subconjunctival, episcleral, periocular, transscleral, retrobulbar, posterior juxtascleral, circumcorneal, or tear duct injections, or through a mucus, mucin, or a mucosal barrier, in an immediate or controlled release fashion or via an ocular device.

Parenteral formulations can be prepared in conventional forms, either as liquid solutions or suspensions, solid forms suitable for solubilization or suspension in liquid prior to injection, or as emulsions. Typically, sterile injectable suspensions are formulated according to techniques known in the art using suitable carriers, dispersing or wetting agents and suspending agents. The sterile injectable formulation can also be a sterile injectable solution or a suspension in a acceptably nontoxic parenterally acceptable diluent or solvent. Among the acceptable vehicles and solvents that can be employed are water, Ringer's solution and isotonic sodium chloride solution. In addition, sterile, fixed oils, fatty esters or polyols are conventionally employed as solvents or suspending media. In addition, parenteral administration can involve the use of a slow release or sustained release system such that a constant level of dosage is maintained.

Parenteral administration includes intraarticular, intravenous, intramuscular, intradermal, intraperitoneal, and subcutaneous routes, and include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Administration via certain parenteral routes can involve introducing the formulations of the disclosure into the body of a patient through a needle or a catheter, propelled by a sterile syringe or some other mechanical device such as a continuous infusion system. A formulation provided by the disclosure can be administered using a syringe, injector, pump, or any other device recognized in the art for parenteral administration.

Preparations according to the disclosure for parenteral administration include sterile aqueous or non-aqueous solutions, suspensions, or emulsions. Examples of non-aqueous solvents or vehicles are propylene glycol, polyethylene glycol, vegetable oils, such as olive oil and corn oil, gelatin, and injectable organic esters such as ethyl oleate. Such dosage forms can also contain adjuvants such as preserving, wetting, emulsifying, and dispersing agents. They can be sterilized by, for example, filtration through a bacteria retaining filter, by incorporating sterilizing agents into the compositions, by irradiating the compositions, or by heating the compositions. They can also be manufactured using sterile water, or some other sterile injectable medium, immediately before use.

Sterile injectable solutions are prepared by incorporating one or more of the compounds of the disclosure in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, typical methods of preparation are vacuum-drying and freeze-drying techniques which yield a powder of the active ingredient plus any additional desired ingredient from a previously sterile-filtered solution thereof. Thus, for example, a parenteral composition suitable for administration by injection is prepared by stirring 1.5% by weight of active ingredient in 10% by volume propylene glycol and water. The solution is made isotonic with sodium chloride and sterilized.

Alternatively, the pharmaceutical compositions of the disclosure can be administered in the form of suppositories for rectal administration. These can be prepared by mixing the agent with a suitable nonirritating excipient which is solid at room temperature but liquid at the rectal temperature and therefore will melt in the rectum to release the drug. Such materials include cocoa butter, beeswax and polyethylene glycols.

The pharmaceutical compositions of the disclosure can also be administered by nasal aerosol or inhalation. Such compositions are prepared according to techniques well-known in the art of pharmaceutical formulation and can be prepared as solutions in saline, employing benzyl alcohol or other suitable preservatives, absorption promoters to enhance bioavailability, propellants such as fluorocarbons or nitrogen, and/or other conventional solubilizing or dispersing agents.

Formulations for buccal administration include tablets, lozenges, gels and the like. Alternatively, buccal administration can be effected using a transmucosal delivery system as known to those skilled in the art. The compounds of the disclosure can also be delivered through the skin or muscosal tissue using conventional transdermal drug delivery systems, i.e., transdermal “patches” wherein the agent is typically contained within a laminated structure that serves as a drug delivery device to be affixed to the body surface. In such a structure, the drug composition is typically contained in a layer, or “reservoir,” underlying an upper backing layer. The laminated device can contain a single reservoir, or it can contain multiple reservoirs. In certain embodiments, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like.

Alternatively, the drug-containing reservoir and skin contact adhesive are present as separate and distinct layers, with the adhesive underlying the reservoir which, in this case, can be either a polymeric matrix as described above, or it can be a liquid or gel reservoir, or can take some other form. The backing layer in these laminates, which serves as the upper surface of the device, functions as the primary structural element of the laminated structure and provides the device with much of its flexibility. The material selected for the backing layer should be substantially impermeable to the active agent and any other materials that are present.

The compositions of the disclosure can be formulated for aerosol administration, particularly to the respiratory tract and including intranasal administration. The compound may, for example generally have a small particle size for example of the order of 5 microns or less. Such a particle size can be obtained by means known in the art, for example by micronization. The active ingredient is provided in a pressurized pack with a suitable propellant such as a chlorofluorocarbon (CFC) for example dichlorodifluoromethane, trichlorofluoromethane, or dichlorotetrafluoroethane, carbon dioxide or other suitable gas. The aerosol can conveniently also contain a surfactant such as lecithin. The dose of drug can be controlled by a metered valve.

Alternatively, the active ingredients can be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition can be presented in unit dose form for example in capsules or cartridges of e.g., gelatin or blister packs from which the powder can be administered by means of an inhaler.

Formulations suitable for rectal administration are typically presented as unit dose suppositories. These may be prepared by admixing the active compound with one or more conventional solid carriers, for example, cocoa butter, and then shaping the resulting mixture.

In certain embodiments, the pharmaceutical composition is suitable for topical application to the skin using a mode of administration and defined above.

In certain embodiments, the pharmaceutical composition is suitable for transdermal administration may be presented as discrete patches adapted to remain in intimate contact with the epidermis of the recipient for a prolonged period of time. Formulations suitable for transdermal administration may also be delivered by iontophoresis (see, for example, Pharmaceutical Research 3 (6):318 (1986)) and typically take the form of an optionally buffered aqueous solution of the active compound.

In certain embodiments, microneedle patches or devices are provided for delivery of drugs across or into biological tissue, particularly the skin. The microneedle patches or devices permit drug delivery at clinically relevant rates across or into skin or other tissue barriers, with minimal or no damage, pain, or irritation to the tissue.

Formulations suitable for administration to the lungs can be delivered by a wide range of passive breath driven and active power driven single/-multiple dose dry powder inhalers (DPI). The devices most commonly used for respiratory delivery include nebulizers, metered-dose inhalers, and dry powder inhalers. Several types of nebulizers are available, including jet nebulizers, ultrasonic nebulizers, and vibrating mesh nebulizers. Selection of a suitable lung delivery device depends on parameters, such as nature of the drug and its formulation, the site of action, and pathophysiology of the lung.

In certain embodiments an oral formulation is provided.

### Example A

Tablets of the following composition are manufactured in the usual manner:

TABLE 1
possible tablet composition

| | mg/tablet | | | |
|---|---|---|---|---|
| ingredient | 5 | 25 | 100 | 500 |
| Compound of Formula I, II, III, or | 5 | 25 | 100 | 500 |
| IV | | | | |
| Lactose Anhydrous DTG | 125 | 105 | 30 | 150 |
| Sta-Rx 1500 | 6 | 6 | 6 | 60 |
| Microcrystalline Cellulose | 30 | 30 | 30 | 450 |
| Magnesium Stearate | 1 | 1 | 1 | 1 |
| Total | 167 | 167 | 167 | 831 |

### Manufacturing Procedure

1. Mix ingredients 1, 2, 3 and 4 and granulate with purified water.
2. Dry the granules at 50° C.
3. Pass the granules through suitable milling equipment.
4. Add ingredient 5 and mix for three minutes; compress on a suitable press.

### Example B-1

Capsules of the following composition are manufactured:

TABLE 2
possible capsule ingredient composition

| | mg/capsule | | | |
|---|---|---|---|---|
| ingredient | 5 | 25 | 100 | 500 |
| Compound of Formula I, II, III, or | 5 | 25 | 100 | 500 |
| IV | | | | |
| Hydrous Lactose | 159 | 123 | 148 | — |
| Corn Starch | 25 | 35 | 40 | 70 |
| Talk | 10 | 15 | 10 | 25 |
| Magnesium Stearate | 1 | 2 | 2 | 5 |
| Total | 200 | 200 | 300 | 600 |

### Manufacturing Procedure

1. Mix ingredients 1, 2 and 3 in a suitable mixer for 30 minutes.
2. Add ingredients 4 and 5 and mix for 3 minutes.
3. Fill into a suitable capsule.

The compound of Formula I, II, III, or IV, lactose and corn starch are firstly mixed in a mixer and then in a comminuting machine. The mixture is returned to the mixer; the talc is added thereto and mixed thoroughly. The mixture is filled by machine into suitable capsules, e.g. hard gelatin capsules.

### Example B-2

Soft Gelatin Capsules of the following composition are manufactured:

TABLE 3
possible soft gelatin capsule ingredient composition

| ingredient | mg/capsule |
|---|---|
| Compound of Formula I, II, III, or IV | 5 |
| Yellow wax | 8 |
| Hydrogenated Soya bean oil | 8 |
| Partially hydrogenated plant oils | 34 |
| Soya bean oil | 110 |
| Total | 165 |

TABLE 4
possible soft gelatin capsule composition

| ingredient | mg/capsule |
|---|---|
| Gelatin | 75 |
| Glycerol 85% | 32 |
| Karion 83 | 8 (dry matter) |
| Titan dioxide | 0.4 |
| Iron oxide yellow | 1.1 |
| Total | 116.5 |

### Manufacturing Procedure

The compound of Formula I, II, III, or IV is dissolved in a warm melting of the other ingredients and the mixture is filled into soft gelatin capsules of appropriate size. The filled soft gelatin capsules are treated according to the usual procedures.

### Example C

Suppositories of the following composition are manufactured:

TABLE 5
possible suppository composition

| ingredient | mg/supp. |
|---|---|
| Compound of Formula I, II, III, or IV | 15 |
| Suppository mass | 1285 |
| Total | 1300 |

### Manufacturing Procedure

The suppository mass is melted in a glass or steel vessel, mixed thoroughly and cooled to 45° C. Thereupon, the finely powdered compound of Formula I, II, III, or IV is added thereto and stirred until it has dispersed completely. The mixture is poured into suppository moulds of suitable size, left to cool; the suppositories are then removed from the moulds and packed individually in wax paper or metal foil.

### Example D

Injection solutions of the following composition are manufactured:

TABLE 6
possible injection solution composition

| ingredient | mg/injection solution. |
|---|---|
| Compound of Formula I, II, III, or IV | 3 |
| Polyethylene Glycol 400 | 150 |
| acetic acid | q.s. ad pH 5.0 |
| water for injection solutions | ad 1.0 ml |

### Manufacturing Procedure

The compound of Formula I, II, III, or IV is dissolved in a mixture of Polyethylene Glycol 400 and water for injection (part). The pH is adjusted to 5.0 by acetic acid. The volume is adjusted to 1.0 ml by addition of the residual amount of water. The solution is filtered, filled into vials using an appropriate overage and sterilized.

### Example E

Sachets of the following composition are manufactured:

TABLE 7
possible sachet composition

| ingredient | mg/sachet |
|---|---|
| Compound of Formula I, II, III, or IV | 50 |
| Lactose, fine powder | 1015 |
| Microcrystalline cellulose (AVICEL PH 102) | 1400 |
| Sodium carboxymethyl cellulose | 14 |
| Polyvinylpyrrolidon K 30 | 10 |
| Magnesium stearate | 10 |
| Flavoring additives | 1 |
| Total | 2500 |

### Manufacturing Procedure

The compound of Formula I, II, III, or IV is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

### VIII. Pharmacological Tests

The compounds of Formula I, II, III, or IV and their pharmaceutically acceptable salts possess valuable pharmacological properties. The compounds were investigated in accordance with the test given hereinafter.

### Materials

NCI-H1975 (harboring EGFR heterozygous L858R-T790M mutations) and NCI-H3255 (harboring EGFR heterozygous L858R mutation) were purchased from ATCC and NCI, respectively. NCI-H1975+CS (harboring EGFR heterozygous L858R-T790M-C797S mutations) was generated using CRISPR technology to introduce the additional C797S mutation by Horizon Discovery. A431 (harboring EGFR wildtype) was purchased from ATCC. RPMI 1640 no-phenol red medium and fetal bovine serum (FBS) were purchased from Gibco (Grand Island, NY, USA). Cell culture flasks and 384-well microplates were acquired from VWR (Radnor, PA, USA). Phosphorylated (pY1068) EGFR and Total EGFR (using L858R-specific detection antibody and pan-EGFR antibody for EGFR mutant cell lines and EGFR wild-type cell lines, respectively) HTRF assay kits were purchased from Cisbio (Bedford, MA, USA).

### EGFR Inhibition and Degradation Analysis

Degradation of EGFR protein containing L858R mutation or wild-type was determined based on quantification of FRET signal using a Total EGFR (L858R-specific or pan-EGFR detecting) HTRF assay kit. Phospho-EGFR (pEGFR) inhibition was determined based on quantification of FRET signal using a pY1068 EGFR HTRF assay kit. In separate assay plates, test compounds were added to the 384-well plate from a top concentration of 10 μM with 11 points, half log titration in duplicates. For each assay, 12.5 uL of cells suspended in assay media (RPMI 1640 no-phenol red medium+10% FBS) at cell densities indicated for each cell line in Table 8 below were dispensed using a multi-channel pipette to 384-well low volume white HTRF microplates containing a duplicate concentration range of test compounds and DMSO controls. The plates were kept at 37° C. with 5% CO2 for 6 hours and then incubated with either phospho EGFR or Total EGFR HTRF detection antibodies according to the cell line and EGFR mutant being assayed. Cells treated in the absence of the test compound were the negative control. Positive control was set by wells containing all reagents but no cells. FRET signal was acquired on EnVision™ Multilabel Reader (PerkinElmer, Santa Clara, CA, USA). Compound concentration that achieves 50% degradation and inhibition was reported as DC50 and IC50, respectively.

TABLE 8
EGFR mutant cancer cell lines: EGFR mutation, source vendor,
experimental seeding density.

| | | | Seeding Density | |
|---|---|---|---|---|
| | | | (Cells/Well) | |
| | | | Total | pY1068 |
| Cell Line | Mutation | Vendor | EGFR | EGFR |
| A431 | EGFR WT | ATCC | 2000 | 10000 |
| NCI-H3255 | EGFR L858R | NCI | 1000 | 5000 |
| NCI-H1975 | EGFR L858R-T790M | ATCC | 10000 | 10000 |
| NCI-H1975 + | EGFR L858R- | Horizon | 20000 | 20000 |
| CS | T790M-L797S | (CRISPR-in) | | |

TABLE 9A
Potencies of EGFR mutant protein degradation

| | | | | H1975+ |
|---|---|---|---|---|
| | | H1975 | H3255 | CS |
| | | EGFR | EGFR | EGFR |
| | | DC50 | DC50 | DC50 |
| Compound | | [nM] | [nM] | [nM] |
| 1 | 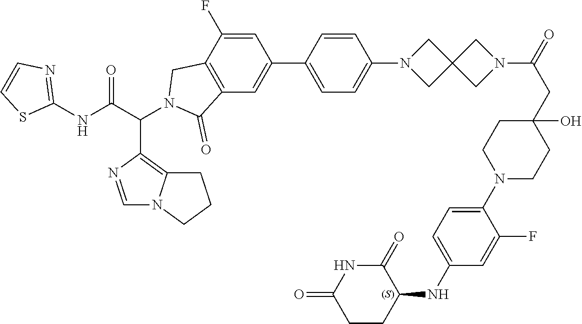 | *** | *** | *** |
| | 2-(6,7-dihydro-5H-pyrrolo[1,2-c]imidazol-1-yl)-2-(6-(4-(6-(2- | | | |
| | (1-(4-(((S)-2,6-dioxopiperidin-3-yl)amino)-2-fluorophenyl)-4- | | | |
| | hydroxypiperidin-4-yl)acetyl)-2,6-diazaspiro[3.3]heptan-2- | | | |
| | yl)phenyl)-4-fluoro-1-oxoisoindolin-2-yl)-N-(thiazol-2- | | | |
| | yl)acetamide | | | |
| 2 | 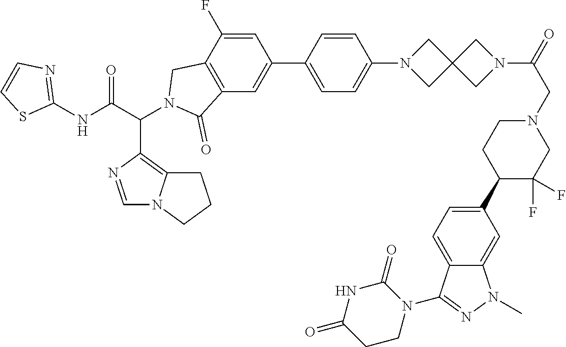 [SMILES: Cn1nc(N2CCC(=O)NC2=O)c2ccc([C@H]3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21] | *** | *** | *** |
| | 2-(6,7-dihydro-5H-pyrrolo[1,2-c]imidazol-1-yl)-2-(6-(4-(6-(2- | | | |
| | ((R)-4-(3-(2,4-dioxotetrahydropyrimidin-1(2H)-yl)-1-methyl- | | | |
| | 1H-indazol-6-yl)-3,3-difluoropiperidin-1-yl)acetyl)-2,6- | | | |
| | diazaspiro[3.3]heptan-2-yl)phenyl)-4-fluoro-1-oxoisoindolin- | | | |
| | 2-yl)-N-(thiazol-2-yl)acetamide | | | |
| 3 | 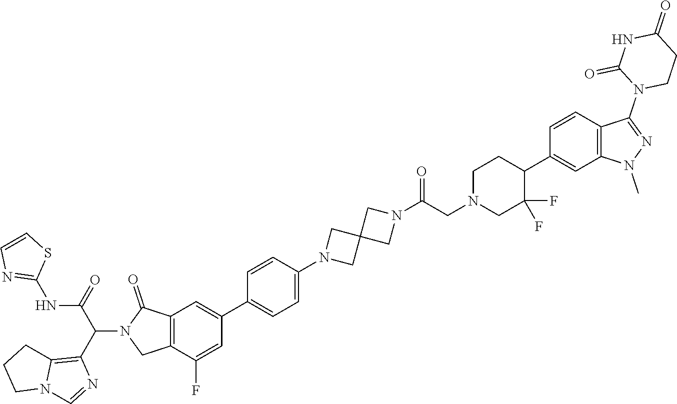 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21] | *** | | |
| | 2-(6,7-dihydro-5H-pyrrolo[1,2-c]imidazol-1-yl)-2-[6-[4-[2-[2- | | | |
| | [4-[3-(2,4-dioxohexahydropyrimidin-1-yl)-1-methyl-indazol- | | | |
| | 6-yl]-3,3-difluoro-1-piperidyl]acetyl]-2,6- | | | |
| | diazaspiro[3.3]heptan-6-yl]phenyl]-4-fluoro-1-oxo-isoindolin- | | | |
| | 2-yl]-N-thiazol-2-yl-acetamide | | | |
| 4 | 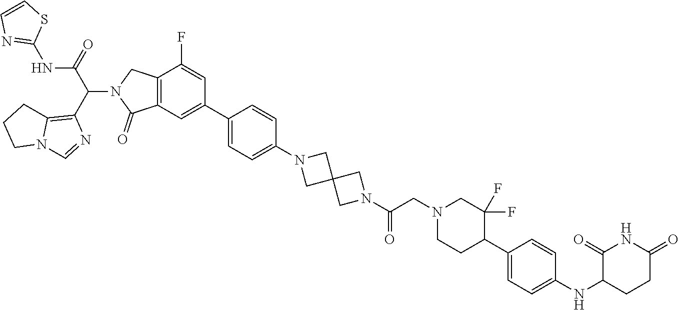 [SMILES: O=C1CCC(Nc2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc2)C(=O)N1] | *** | | |
| | 2-(6,7-dihydro-5H-pyrrolo[1,2-c]imidazol-1-yl)-2-(6-(4-(6-(2- | | | |
| | (4-(4-((2,6-dioxopiperidin-3-yl)amino)phenyl)-3,3- | | | |
| | difluoropiperidin-1-yl)acetyl)-2,6-diazaspiro[3.3]heptan-2- | | | |
| | yl)phenyl)-4-fluoro-1-oxoisoindolin-2-yl)-N-(thiazol-2- | | | |
| | yl)acetamide, Isomer 1 | | | |
| 5 | 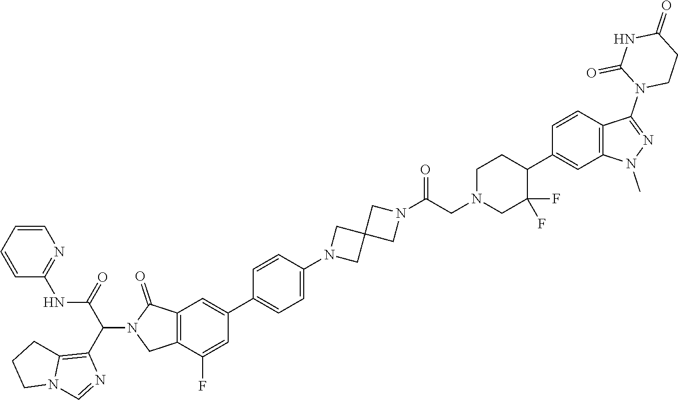 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6ccccn6)c6ncn8c6CCC8)C7)cc4)C5)CC3(F)F)cc21] | *** | | |
| | 2-(6,7-dihydro-5H-pyrrolo[1,2-c]imidazol-1-yl)-2-[6-[4-[2-[2- | | | |
| | [4-[3-(2,4-dioxohexahydropyrimidin-1-yl)-1-methyl-indazol- | | | |
| | 6-yl]-3,3-difluoro-1-piperidyl]acetyl]-2,6- | | | |
| | diazaspiro[3.3]heptan-6-yl]phenyl]-4-fluoro-1-oxo-isoindolin- | | | |
| | 2-yl]-N-(2-pyridyl)acetamide | | | |
| 6 | 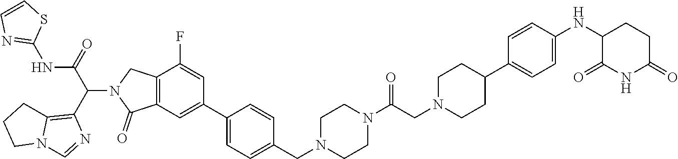 [SMILES: O=C1CCC(Nc2ccc(C3CCN(CC(=O)N4CCN(Cc5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | |
| | 2-(6,7-dihydro-5H-pyrrolo[1,2-c]imidazol-1-yl)-2-[6-[4-[[4- | | | |
| | [2-[4-[4-[(2,6-dioxo-3-piperidyl)amino]phenyl]-1- | | | |
| | piperidyl]acetyl]piperazin-1-yl]methyl]phenyl]-4-fluoro-1- | | | |
| | oxo-isoindolin-2-yl]-N-thiazol-2-yl-acetamide | | | |
| 7 | 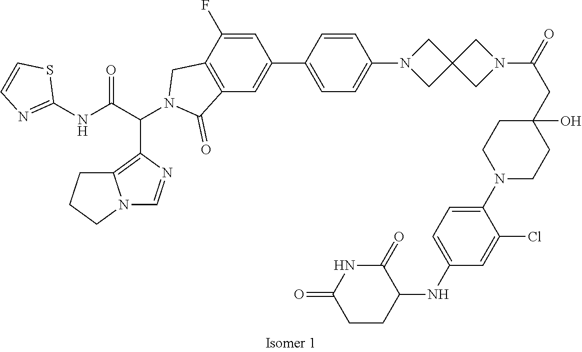 [SMILES: O=C1CCC(Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1] | *** | | |
| | 2-(6-(4-(6-(2-(1-(2-chloro-4-((2,6-dioxopiperidin-3- | | | |
| | yl)amino)phenyl)-4-hydroxypiperidin-4-yl)acetyl)-2,6- | | | |
| | diazaspiro[3.3]heptan-2-yl)phenyl)-4-fluoro-1-oxoisoindolin- | | | |
| | 2-yl)-2-(6,7-dihydro-5H-pyrrolo[1,2-c]imidazol-1-yl)-N- | | | |
| | (thiazol-2-yl)acetamide, isomer 1 | | | |
| 8 | 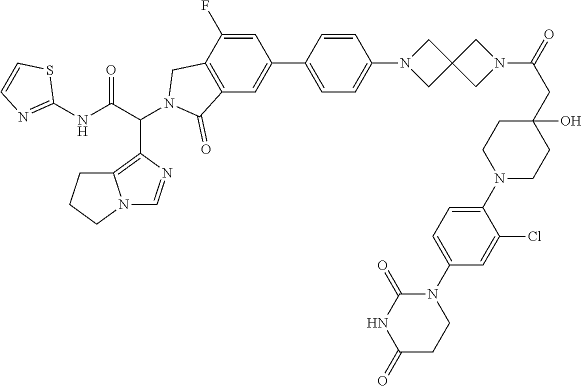 [SMILES: O=C1CCN(c2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1] | *** | | |
| | 2-[6-[4-[2-[2-[1-[2-chloro-4-(2,4-dioxohexahydropyrimidin-1- | | | |
| | yl)phenyl]-4-hydroxy-4-piperidyl]acetyl]-2,6- | | | |
| | diazaspiro[3.3]heptan-6-yl]phenyl]-4-fluoro-1-oxo-isoindolin- | | | |
| | 2-yl]-2-(6,7-dihydro-5H-pyrrolo[1,2-c]imidazol-1-yl)-N- | | | |
| | thiazol-2-yl-acetamide | | | |
| 9 | 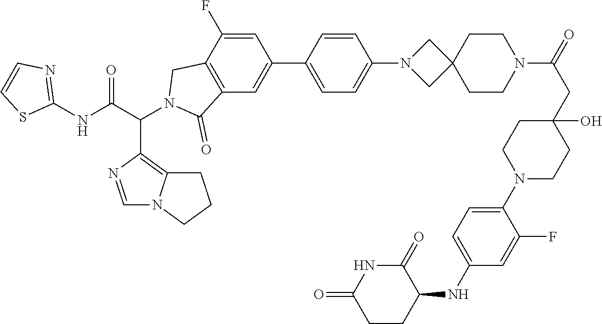 [SMILES: O=C1CC[C@H](Nc2ccc(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-[6-[4-[7-[2- | | | |
| | [1-[4-[[(3S)-2,6-dioxo-3-piperidyl]amino]-2-fluoro-phenyl]-4- | | | |
| | hydroxy-4-piperidyl]acetyl]-2,7-diazaspiro[3.5]nonan-2- | | | |
| | yl]phenyl]-4-fluoro-1-oxo-isoindolin-2-yl]-N-thiazol-2-yl- | | | |
| | acetamide | | | |
| 10 | 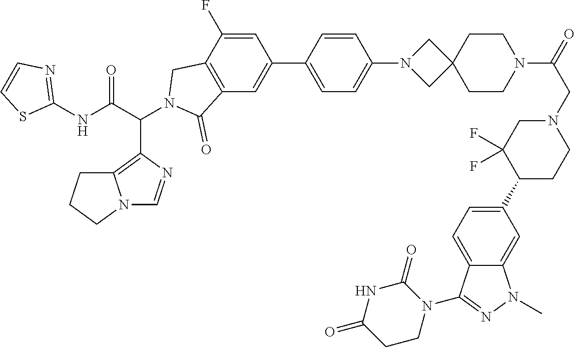 [SMILES: Cn1nc(N2CCC(=O)NC2=O)c2ccc([C@H]3CCN(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3(F)F)cc21] | *** | | |
| | 2-(6,7-dihydro-5H-pyrrolo[1,2-c]imidazol-1-yl)-2-[6-[4-[7-[2- | | | |
| | [(4R)-4-[3-(2,4-dioxohexahydropyrimidin-1-yl)-1-methyl- | | | |
| | indazol-6-yl]-3,3-difluoro-1-piperidyl]acetyl]-2,7- | | | |
| | diazaspiro[3.5]nonan-2-yl]phenyl]-4-fluoro-1-oxo-isoindolin- | | | |
| | 2-yl]-N-thiazol-2-yl-acetamide | | | |
| 11 | 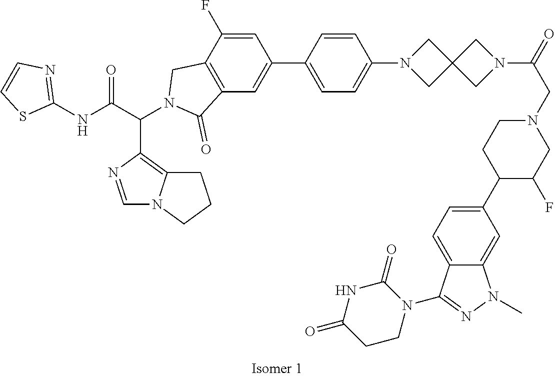 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3F)cc21] | *** | | |
| | 2-(6,7-dihydro-5H-pyrrolo[1,2-c]imidazol-1-yl)-2-[6-[4-[2-[2- | | | |
| | [4-[3-(2,4-dioxohexahydropyrimidin-1-yl)-1-methyl-indazol- | | | |
| | 6-yl]-3-fluoro-1-piperidyl]acetyl]-2,6-diazaspiro[3.3]heptan-6- | | | |
| | yl]phenyl]-4-fluoro-1-oxo-isoindolin-2-yl]-N-thiazol-2-yl- | | | |
| | acetamide, isomer 1 | | | |
| 12 | 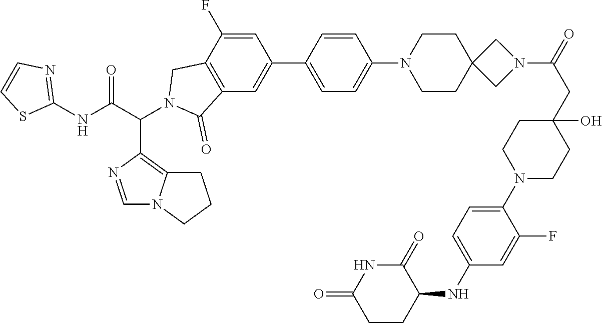 [SMILES: O=C1CC[C@H](Nc2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1] | *** | | |
| | 2-(6,7-dihydro-5H-pyrrolo[1,2-c]imidazol-1-yl)-2-[6-[4-[2-[2- | | | |
| | [1-[4-[(3S)-2,6-dioxo-3-piperidyl]amino]-2-fluoro-phenyl]-4- | | | |
| | hydroxy-4-piperidyl]acetyl]-2,7-diazaspiro[3.5]nonan-7- | | | |
| | yl]phenyl]-4-fluoro-1-oxo-isoindolin-2-yl]-N-thiazol-2-yl- | | | |
| | acetamide | | | |
| 13 | 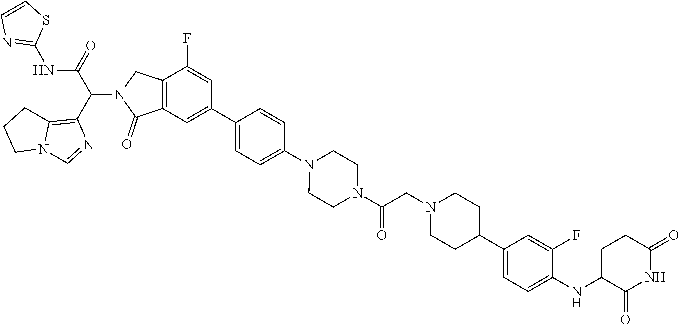 [SMILES: O=C1CCC(Nc2ccc(C3CCN(CC(=O)N4CCN(c5ccc(-c6cc(F)c7c(c6)C(=O)N(C(C(=O)Nc6nccs6)c6ncn8c6CCC8)C7)cc5)CC4)CC3)cc2F)C(=O)N1] | ** | | |
| | 2-(6,7-dihydro-5H-pyrrolo[1,2-c]imidazol-1-yl)-2-[6-[4-[4-[2- | | | |
| | [4-[4-[(2,6-dioxo-3-piperidyl)amino]-3-fluoro-phenyl]-1- | | | |
| | piperidyl]acetyl]piperazin-1-yl]phenyl]-4-fluoro-1-oxo- | | | |
| | isoindolin-2-yl]-N-thiazol-2-yl-acetamide | | | |
| 14 | 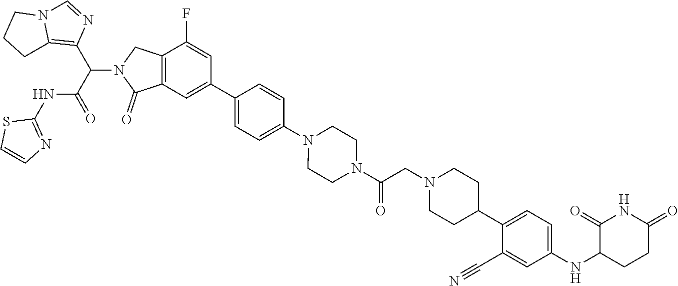 [SMILES: N#Cc1cc(NC2CCC(=O)NC2=O)ccc1C1CCN(CC(=O)N2CCN(c3ccc(-c4cc(F)c5c(c4)C(=O)N(C(C(=O)Nc4nccs4)c4ncn6c4CCC6)C5)cc3)CC2)CC1] | *** | *** | *** |
| | 2-[6-[4-[4-[2-[4-[2-cyano-4-[(2,6-dioxo-3- | | | |
| | piperidyl)amino]phenyl]-1-piperidyl]acetyl]piperazin-1- | | | |
| | yl]phenyl]-4-fluoro-1-oxo-isoindolin-2-yl]-2-(6,7-dihydro- | | | |
| | 5H-pyrrolo[1,2-c]imidazol-1-yl)-N-thiazol-2-yl-acetamide | | | |
| 15 | 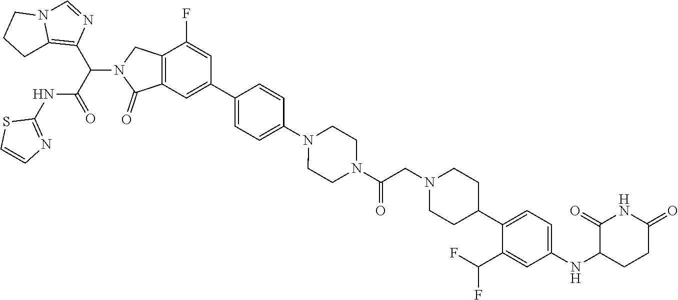 [SMILES: O=C1CCC(Nc2ccc(C3CCN(CC(=O)N4CCN(c5ccc(-c6cc(F)c7c(c6)C(=O)N(C(C(=O)Nc6nccs6)c6ncn8c6CCC8)C7)cc5)CC4)CC3)c(C(F)F)c2)C(=O)N1] | *** | *** | *** |
| | 2-[6-[4-[4-[2-[4-[2-(difluoromethyl)-4-[(2,6-dioxo-3- | | | |
| | piperidyl)amino]phenyl]-1-piperidyl]acetyl]piperazin-1- | | | |
| | yl]phenyl]-4-fluoro-1-oxo-isoindolin-2-yl]-2-(6,7-dihydro- | | | |
| | 5H-pyrrolo[1,2-c]imidazol-1-yl)-N-thiazol-2-yl-acetamide | | | |
| 16 | 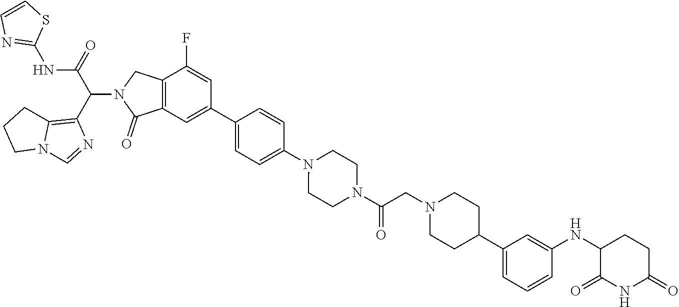 [SMILES: O=C1CCC(Nc2cccc(C3CCN(CC(=O)N4CCN(c5ccc(-c6cc(F)c7c(c6)C(=O)N(C(C(=O)Nc6nccs6)c6ncn8c6CCC8)C7)cc5)CC4)CC3)c2)C(=O)N1] | ** | | |
| | 2-(6,7-dihydro-5H-pyrrolo[1,2-c]imidazol-1-yl)-2-(6-(4-(4-(2- | | | |
| | (4-(3-((2,6-dioxopiperidin-3-yl)amino)phenyl)piperidin-1- | | | |
| | yl)acetyl)piperazin-1-yl)phenyl)-4-fluoro-1-oxoisoindolin-2- | | | |
| | yl)-N-(thiazol-2-yl)acetamide | | | |
| 17 | 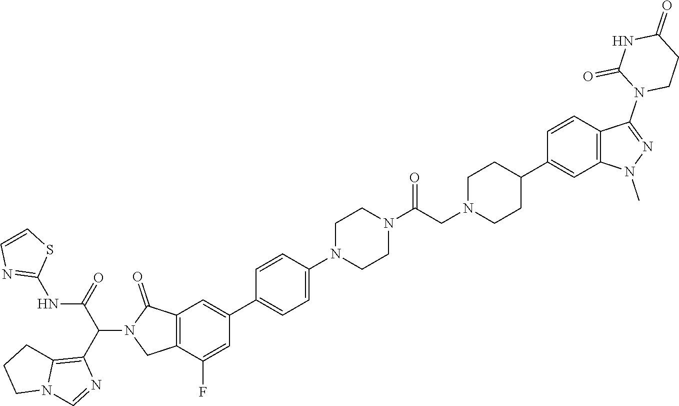 [SMILES: Cn1nc(N2CCC(=O)NC2=O)c2ccc(C3CCN(CC(=O)N4CCN(c5ccc(-c6cc(F)c7c(c6)C(=O)N(C(C(=O)Nc6nccs6)c6ncn8c6CCC8)C7)cc5)CC4)CC3)cc21] | *** | *** | *** |
| | 2-(6,7-dihydro-5H-pyrrolo[1,2-c]imidazol-1-yl)-2-(6-(4-(4-(2- | | | |
| | (4-(3-(2,4-dioxotetrahydropyrimidin-1(2H)-yl)-1-methyl-1H- | | | |
| | indazol-6-yl)piperidin-1-yl)acetyl)piperazin-1-yl)phenyl)-4- | | | |
| | fluoro-1-oxoisoindolin-2-yl)-N-(thiazol-2-yl)acetamide | | | |
| 18 | 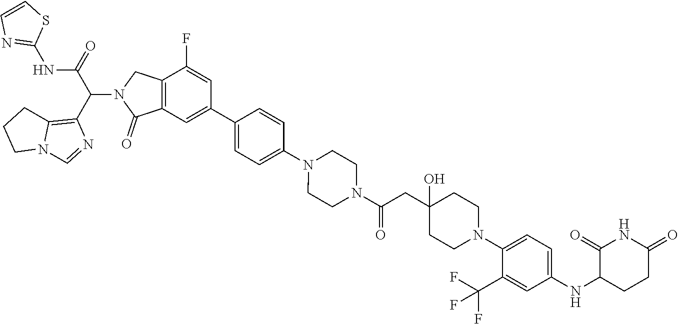 [SMILES: O=C1CCC(Nc2ccc(N3CCC(O)(CC(=O)N4CCN(c5ccc(-c6cc(F)c7c(c6)C(=O)N(C(C(=O)Nc6nccs6)c6ncn8c6CCC8)C7)cc5)CC4)CC3)c(C(F)(F)F)c2)C(=O)N1] | *** | | |
| | 2-(6,7-dihydro-5H-pyrrolo[1,2-c]imidazol-1-yl)-2-(6-(4-(4-(2- | | | |
| | (1-(4-((2,6-dioxopiperidin-3-yl)amino)-2- | | | |
| | (trifluoromethyl)phenyl)-4-hydroxypiperidin-4- | | | |
| | yl)acetyl)piperazin-1-yl)phenyl)-4-fluoro-1-oxoisoindolin-2- | | | |
| | yl)-N-(thiazol-2-yl)acetamide | | | |
| 19 | 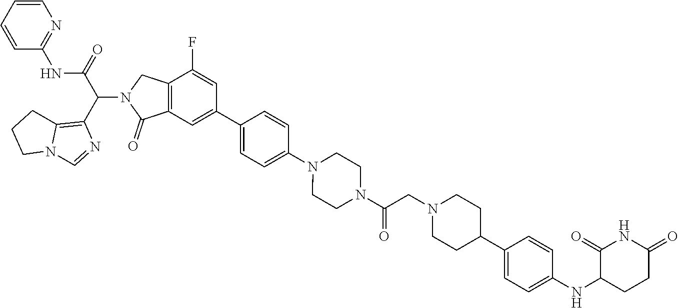 [SMILES: O=C1CCC(Nc2ccc(C3CCN(CC(=O)N4CCN(c5ccc(-c6cc(F)c7c(c6)C(=O)N(C(C(=O)Nc6ccccn6)c6ncn8c6CCC8)C7)cc5)CC4)CC3)cc2)C(=O)N1] | ** | *** | *** |
| | 2-(6,7-dihydro-5H-pyrrolo[1,2-c]imidazol-1-yl)-2-(6-(4-(4-(2- | | | |
| | (4-(4-((2,6-dioxopiperidin-3-yl)amino)phenyl)piperidin-1- | | | |
| | yl)acetyl)piperazin-1-yl)phenyl)-4-fluoro-1-oxoisoindolin-2- | | | |
| | yl)-N-(pyridin-2-yl)acetamide | | | |
| 20 | 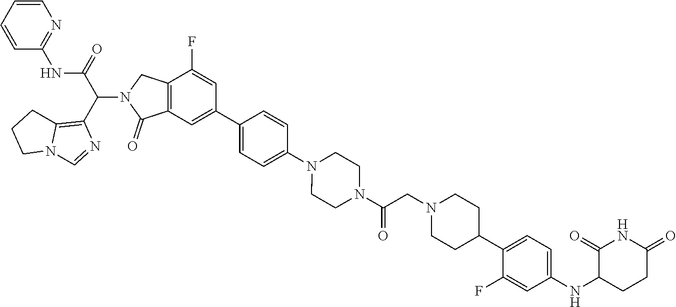 [SMILES: O=C1CCC(Nc2ccc(C3CCN(CC(=O)N4CCN(c5ccc(-c6cc(F)c7c(c6)C(=O)N(C(C(=O)Nc6ccccn6)c6ncn8c6CCC8)C7)cc5)CC4)CC3)c(F)c2)C(=O)N1] | ** | *** | *** |
| | 2-(6,7-dihydro-5H-pyrrolo[1,2-c]imidazol-1-yl)-2-[6-[4-[4-[2- | | | |
| | [4-[4-[(2,6-dioxo-3-piperidyl)amino]-2-fluoro-phenyl]-1- | | | |
| | piperidyl]acetyl]piperazin-1-yl]phenyl]-4-fluoro-1-oxo- | | | |
| | isoindolin-2-yl]-N-(2-pyridyl)acetamide | | | |
| 21 | 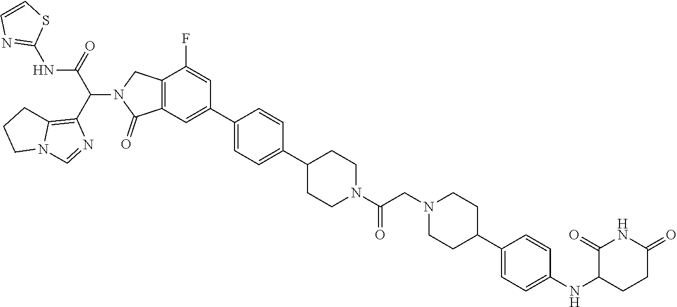 [SMILES: O=C1CCC(Nc2ccc(C3CCN(CC(=O)N4CCC(c5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | *** |
| | 2-(6,7-dihydro-5H-pyrrolo[1,2-c]imidazol-1-yl)-2-(6-(4-(1-(2- | | | |
| | (4-(4-((2,6-dioxopiperidin-3-yl)amino)phenyl)piperidin-1- | | | |
| | yl)acetyl)piperidin-4-yl)phenyl)-4-fluoro-1-oxoisoindolin-2- | | | |
| | yl)-N-(thiazol-2-yl)acetamide | | | |
| 22 | 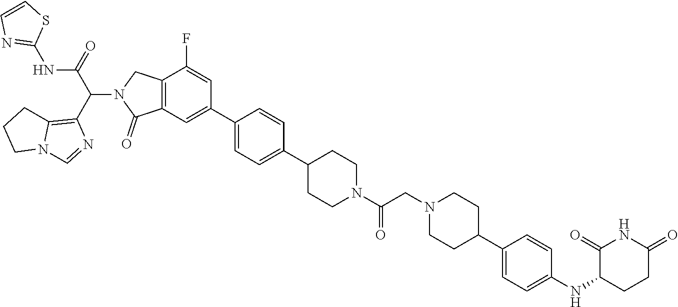 [SMILES: O=C1CC[C@H](Nc2ccc(C3CCN(CC(=O)N4CCC(c5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | |
| | 2-(6,7-dihydro-5H-pyrrolo[1,2-c]imidazol-1-yl)-2-(6-(4-(1-(2- | | | |
| | (4-(4-(((S)-2,6-dioxopiperidin-3-yl)amino)phenyl)piperidin-1- | | | |
| | yl)acetyl)piperidin-4-yl)phenyl)-4-fluoro-1-oxoisoindolin-2- | | | |
| | yl)-N-(thiazol-2-yl)acetamide | | | |
| 23 | 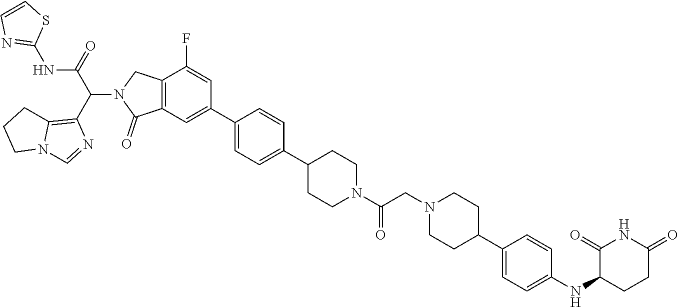 [SMILES: O=C1CC[C@@H](Nc2ccc(C3CCN(CC(=O)N4CCC(c5ccc(-c6cc(F)c7c(c6)C(=O)N(C(C(=O)Nc6nccs6)c6ncn8c6CCC8)C7)cc5)CC4)CC3)cc2)C(=O)N1] | *** | | |
| | 2-(6,7-dihydro-5H-pyrrolo[1,2-c]imidazol-1-yl)-2-(6-(4-(1-(2- | | | |
| | (4-(4-(((R)-2,6-dioxopiperidin-3-yl)amino)phenyl)piperidin-1- | | | |
| | yl)acetyl)piperidin-4-yl)phenyl)-4-fluoro-1-oxoisoindolin-2- | | | |
| | yl)-N-(thiazol-2-yl)acetamide | | | |
| 24 | 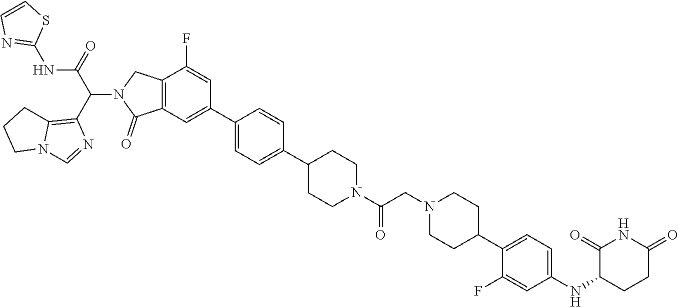 [SMILES: O=C1CC[C@H](Nc2ccc(C3CCN(CC(=O)N4CCC(c5ccc(-c6cc(F)c7c(c6)C(=O)N(C(C(=O)Nc6nccs6)c6ncn8c6CCC8)C7)cc5)CC4)CC3)c(F)c2)C(=O)N1] | ** | *** | *** |
| | 2-(6,7-dihydro-5H-pyrrolo[1,2-c]imidazol-1-yl)-2-(6-(4-(1-(2- | | | |
| | (4-(4-(((S)-2,6-dioxopiperidin-3-yl)amino)-2- | | | |
| | fluorophenyl)piperidin-1-yl)acetyl)piperidin-4-yl)phenyl)-4- | | | |
| | fluoro-1-oxoisoindolin-2-yl)-N-(thiazol-2-yl)acetamide | | | |
| 25 | 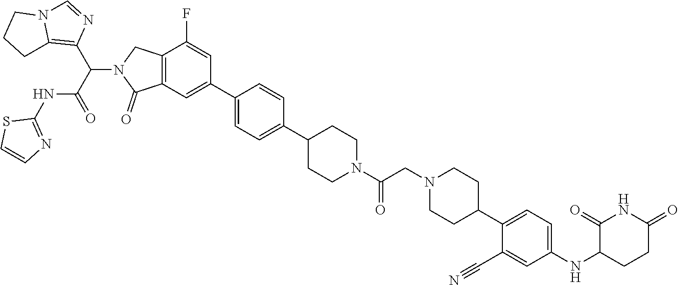 [SMILES: N#Cc1cc(NC2CCC(=O)NC2=O)ccc1C1CCN(CC(=O)N2CCC(c3ccc(-c4cc(F)c5c(c4)C(=O)N(C(C(=O)Nc4nccs4)c4ncn6c4CCC6)C5)cc3)CC2)CC1] | *** | *** | *** |
| | 2-(6-(4-(1-(2-(4-(2-cyano-4-((2,6-dioxopiperidin-3- | | | |
| | yl)amino)phenyl)piperidin-1-yl)acetyl)piperidin-4-yl)phenyl)- | | | |
| | 4-fluoro-1-oxoisoindolin-2-yl)-2-(6,7-dihydro-5H- | | | |
| | pyrrolo[1,2-c]imidazol-1-yl)-N-(thiazol-2-yl)acetamide | | | |
| 26 | 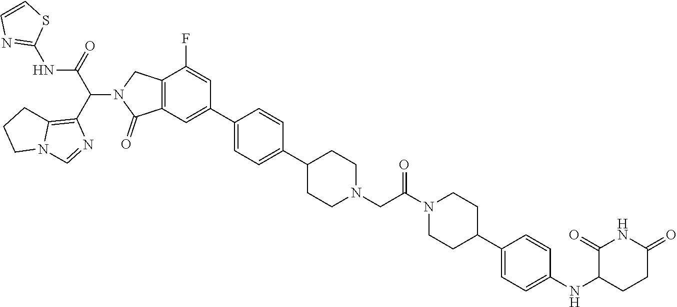 [SMILES: O=C1CCC(Nc2ccc(C3CCN(C(=O)CN4CCC(c5ccc(-c6cc(F)c7c(c6)C(=O)N(C(C(=O)Nc6nccs6)c6ncn8c6CCC8)C7)cc5)CC4)CC3)cc2)C(=O)N1] | *** | | |
| | 2-(6,7-dihydro-5H-pyrrolo[1,2-c]imidazol-1-yl)-2-[6-[4-[1-[2- | | | |
| | [4-[4-[(2,6-dioxo-3-piperidyl)amino]phenyl]-1-piperidyl]-2- | | | |
| | oxo-ethyl]-4-piperidyl]phenyl]-4-fluoro-1-oxo-isoindolin-2- | | | |
| | yl]-N-thiazol-2-yl-acetamide | | | |
| 27 | 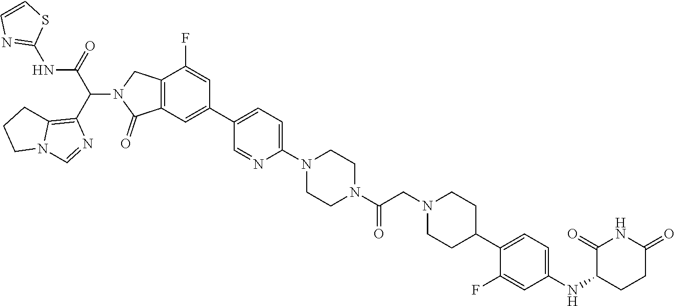 [SMILES: O=C1CC[C@H](Nc2ccc(C3CCN(CC(=O)N4CCN(c5ccc(-c6cc(F)c7c(c6)C(=O)N(C(C(=O)Nc6nccs6)c6ncn8c6CCC8)C7)cn5)CC4)CC3)c(F)c2)C(=O)N1] | *** | *** | |
| | 2-(6,7-dihydro-5H-pyrrolo[1,2-c]imidazol-1-yl)-2-[6-[6-[4-[2- | | | |
| | [4-[4-[(3S)-2,6-dioxo-3-piperidyl]amino]-2-fluoro-phenyl]-1- | | | |
| | piperidyl]acetyl]piperazin-1-yl]-3-pyridyl]-4-fluoro-1-oxo- | | | |
| | isoindolin-2-yl]-N-thiazol-2-yl-acetamide | | | |
| 28 | 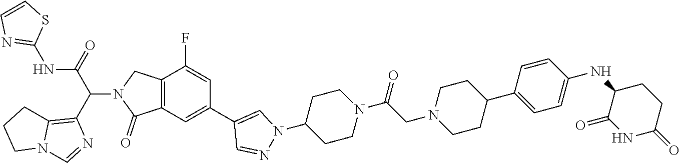 [SMILES: O=C1CC[C@H](Nc2ccc(C3CCN(CC(=O)N4CCC(n5cc(-c6cc(F)c7c(c6)C(=O)N(C(C(=O)Nc6nccs6)c6ncn8c6CCC8)C7)cn5)CC4)CC3)cc2)C(=O)N1] | *** | *** | |
| | 2-(6,7-dihydro-5H-pyrrolo[1,2-c]imidazol-1-yl)-2-[6-[1-[1-[2- | | | |
| | [4-[4-[[(3S)-2,6-dioxo-3-piperidyl]amino]phenyl]-1- | | | |
| | piperidyl]acetyl]-4-piperidyl]pyrazol-4-yl]-4-fluoro-1-oxo- | | | |
| | isoindolin-2-yl]-N-thiazol-2-yl-acetamide | | | |
| 29 | 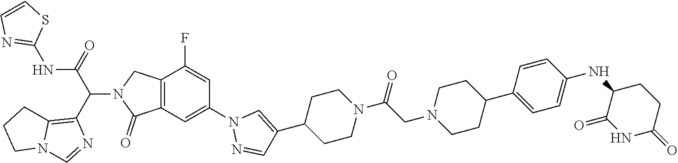 [SMILES: O=C1CC[C@H](Nc2ccc(C3CCN(CC(=O)N4CCC(c5cnn(-c6cc(F)c7c(c6)C(=O)N(C(C(=O)Nc6nccs6)c6ncn8c6CCC8)C7)c5)CC4)CC3)cc2)C(=O)N1] | *** | *** | |
| | 2-(6,7-dihydro-5H-pyrrolo[1,2-c]imidazol-1-yl)-2-[6-[4-[1-[2- | | | |
| | [4-[4-[[(3S)-2,6-dioxo-3-piperidyl]amino]phenyl]-1- | | | |
| | piperidyl]acetyl]-4-piperidyl]pyrazol-1-yl]-4-fluoro-1-oxo- | | | |
| | isoindolin-2-yl]-N-thiazol-2-yl-acetamide | | | |
| 30 | 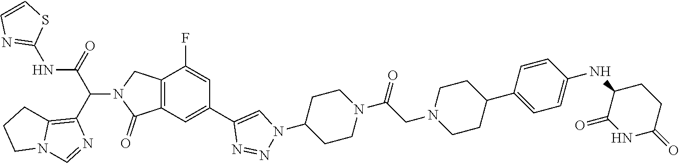 [SMILES: O=C1CC[C@H](Nc2ccc(C3CCN(CC(=O)N4CCC(n5cc(-c6cc(F)c7c(c6)C(=O)N(C(C(=O)Nc6nccs6)c6ncn8c6CCC8)C7)nn5)CC4)CC3)cc2)C(=O)N1] | *** | *** | |
| | 2-(6,7-Dihydro-5H-pyrrolo[1,2-c]imidazol-1-yl)-2-(6-(1-(1- | | | |
| | (2-(4-(4-(((S)-2,6-dioxopiperidin-3- | | | |
| | yl)amino)phenyl)piperidin-1-yl)acetyl)piperidin-4-yl)-1H- | | | |
| | 1,2,3-triazol-4-yl)-4-fluoro-1-oxoisoindolin-2-yl)-N-(thiazol- | | | |
| | 2-yl)acetamide | | | |
| 31 | 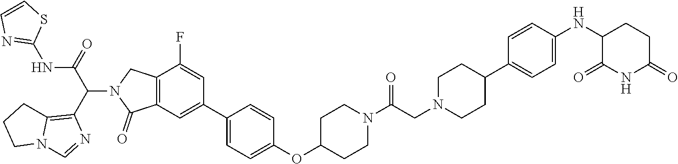 [SMILES: O=C1CCC(Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)cc2)C(=O)N1] | *** | | |
| | 2-(6,7-dihydro-5H-pyrrolo[1,2-c]imidazol-1-yl)-2-[6-[4-[1- | | | |
| | [2-[4-[4-[(2,6-dioxo-3-piperidyl)amino]phenyl]-1- | | | |
| | piperidyl]acetyl]-4-piperidyl]oxy]phenyl]-4-fluoro-1-oxo- | | | |
| | isoindolin-2-yl]-N-thiazol-2-yl-acetamide | | | |
| 32 | 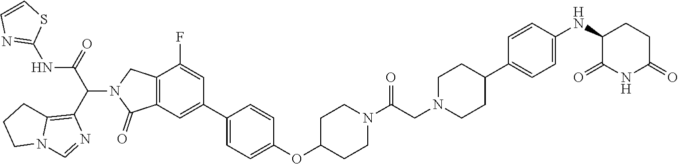 [SMILES: O=C1CC[C@H](Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | *** |
| | 2-(6,7-dihydro-5H-pyrrolo[1,2-c]imidazol-1-yl)-2-[6-[4-[[1- | | | |
| | [2-[4-[4-[[(3S)-2,6-dioxo-3-piperidyl]amino]phenyl]-1- | | | |
| | piperidyl]acetyl]-4-piperidyl]oxy]phenyl]-4-fluoro-1-oxo- | | | |
| | isoindolin-2-yl]-N-thiazol-2-yl-acetamide | | | |
| 33 | 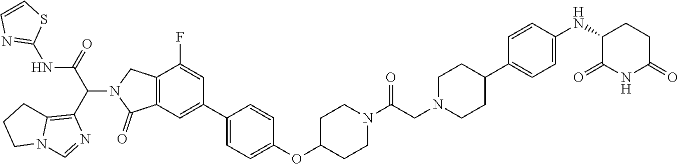 [SMILES: O=C1CC[C@@H](Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | *** |
| | 2-(6,7-dihydro-5H-pyrrolo[1,2-c]imidazol-1-yl)-2-[6-[4-[[1- | | | |
| | [2-[4-[4-[[(3R)-2,6-dioxo-3-piperidyl]amino]phenyl]-1- | | | |
| | piperidyl]acetyl]-4-piperidyl]oxy]phenyl]-4-fluoro-1-oxo- | | | |
| | isoindolin-2-yl]-N-thiazol-2-yl-acetamide | | | |
| 34 | 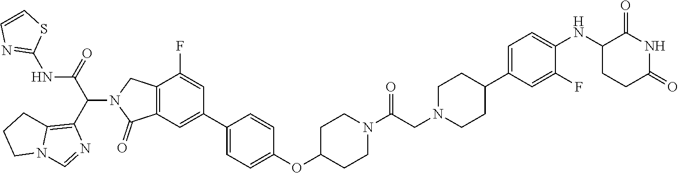 [SMILES: O=C1CCC(Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)cc2F)C(=O)N1] | ** | *** | *** |
| | 2-(6,7-dihydro-5H-pyrrolo[1,2-c]imidazol-1-yl)-2-(6-(4-((1- | | | |
| | (2-(4-(4-((2,6-dioxopiperidin-3-yl)amino)-3- | | | |
| | fluorophenyl)piperidin-1-yl)acetyl)piperidin-4- | | | |
| | yl)oxy)phenyl)-4-fluoro-1-oxoisoindolin-2-yl)-N-(thiazol-2- | | | |
| | yl)acetamide | | | |
| 35 | 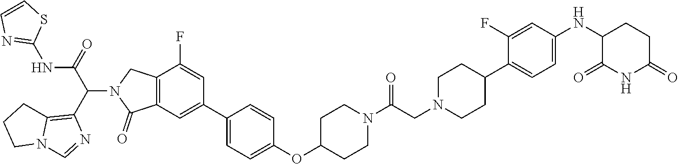 [SMILES: O=C1CCC(Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)c(F)c2)C(=O)N1] | *** | *** | *** |
| | 2-(6,7-dihydro-5H-pyrrolo[1,2-c]imidazol-1-yl)-2-(6-(4-((1- | | | |
| | (2-(4-(4-((2,6-dioxopiperidin-3-yl)amino)-2- | | | |
| | fluorophenyl)piperidin-1-yl)acetyl)piperidin-4- | | | |
| | yl)oxy)phenyl)-4-fluoro-1-oxoisoindolin-2-yl)-N-(thiazol-2- | | | |
| | yl)acetamide | | | |
| 36 | 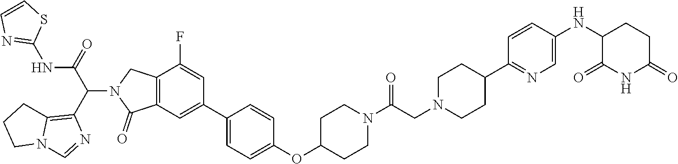 [SMILES: O=C1CCC(Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)nc2)C(=O)N1] | *** | | |
| | 2-(6,7-dihydro-5H-pyrrolo[1,2-c]imidazol-1-yl)-2-(6-(4-((1- | | | |
| | (2-(4-(5-((2,6-dioxopiperidin-3-yl)amino)pyridin-2- | | | |
| | yl)piperidin-1-yl)acetyl)piperidin-4-yl)oxy)phenyl)-4-fluoro- | | | |
| | 1-oxoisoindolin-2-yl)-N-(thiazol-2-yl)acetamide | | | |
| 37 | 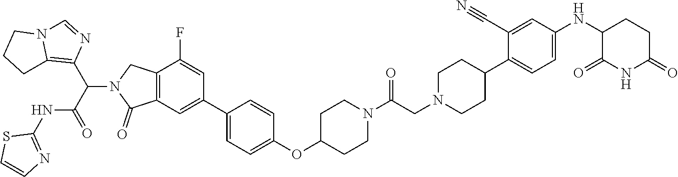 [SMILES: N#Cc1cc(NC2CCC(=O)NC2=O)ccc1C1CCN(CC(=O)N2CCC(Oc3ccc(-c4cc(F)c5c(c4)C(=O)N(C(C(=O)Nc4nccs4)c4ncn6c4CCC6)C5)cc3)CC2)CC1] | *** | *** | *** |
| | 2-(6-(4-((1-(2-(4-(2-cyano-4-((2,6-dioxopiperidin-3- | | | |
| | yl)amino)phenyl)piperidin-1-yl)acetyl)piperidin-4- | | | |
| | yl)oxy)phenyl)-4-fluoro-1-oxoisoindolin-2-yl)-2-(6,7-dihydro- | | | |
| | 5H-pyrrolo[1,2-c]imidazol-1-yl)-N-(thiazol-2-yl)acetamide | | | |
| 38 | 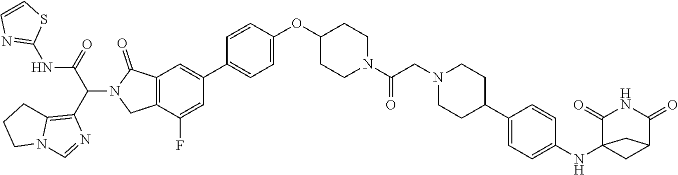 [SMILES: O=C1NC(=O)C2(Nc3ccc(C4CCN(CC(=O)N5CCC(Oc6ccc(-c7cc(F)c8c(c7)C(=O)N(C(C(=O)Nc7nccs7)c7ncn9c7CCC9)C8)cc6)CC5)CC4)cc3)CC1C2] | ** | | |
| | 2-(6,7-dihydro-5H-pyrrolo[1,2-c]imidazol-1-yl)-2-(6-(4-((1- | | | |
| | (2-(4-(4-((2,4-dioxo-3-azabicyclo[3.1.1]heptan-1- | | | |
| | yl)amino)phenyl)piperidin-1-yl)acetyl)piperidin-4- | | | |
| | yl)oxy)phenyl)-4-fluoro-1-oxoisoindolin-2-yl)-N-(thiazol-2- | | | |
| | yl)acetamide | | | |
| 39 | 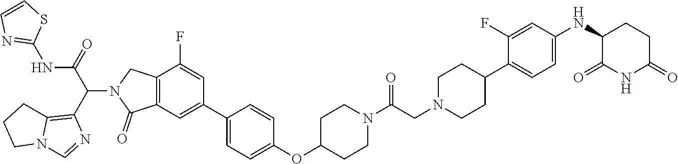 [SMILES: O=C1CC[C@H](Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)c(F)c2)C(=O)N1] | ** | | |
| | 2-(6,7-dihydro-5H-pyrrolo[1,2-c]imidazol-1-yl)-2-(6-(4-((1- | | | |
| | (2-(4-(4-(((S)-2,6-dioxopiperidin-3-yl)amino)-2- | | | |
| | fluorophenyl)piperidin-1-yl)acetyl)piperidin-4- | | | |
| | yl)oxy)phenyl)-4-fluoro-1-oxoisoindolin-2-yl)-N-(thiazol-2- | | | |
| | yl)acetamide | | | |
| 40 | 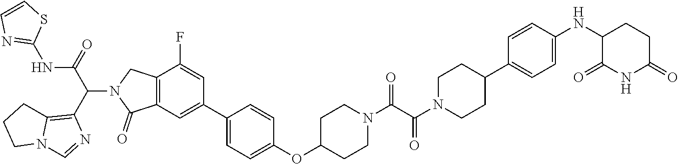 [SMILES: O=C1CCC(Nc2ccc(C3CCN(C(=O)C(=O)N4CCC(Oc5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | |
| | 2-(6,7-dihydro-5H-pyrrolo[1,2-c]imidazol-1-yl)-2-[6-[4-[1- | | | |
| | [2-[4-[4-[(2,6-dioxo-3-piperidyl)amino]phenyl]-1-piperidyl]- | | | |
| | 2-oxo-acetyl]-4-piperidyl]oxy]phenyl]-4-fluoro-1-oxo- | | | |
| | isoindolin-2-yl]-N-thiazol-2-yl-acetamide | | | |
| 41 | 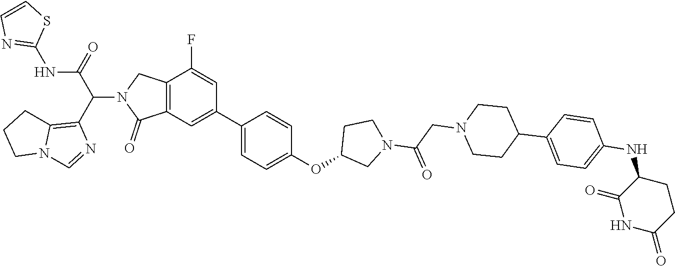 [SMILES: O=C1CC[C@H](Nc2ccc(C3CCN(CC(=O)N4CC[C@@H](Oc5ccc(-c6cc(F)c7c(c6)C(=O)N(C(C(=O)Nc6nccs6)c6ncn8c6CCC8)C7)cc5)C4)CC3)cc2)C(=O)N1] | *** | | |
| | 2-(6,7-dihydro-5H-pyrrolo[1,2-c]imidazol-1-yl)-2-[6-[4- | | | |
| | [(3R)-1-[2-[4-[4-[[(3S)-2,6-dioxo-3-piperidyl]amino]phenyl]- | | | |
| | 1-piperidyl]acetyl]pyrrolidin-3-yl]oxyphenyl]-4-fluoro-1-oxo- | | | |
| | isoindolin-2-yl]-N-thiazol-2-yl-acetamide | | | |
| 42 | 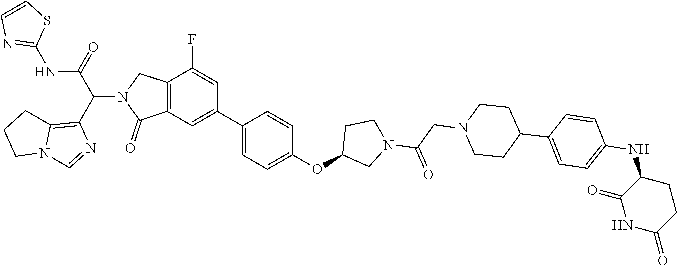 [SMILES: O=C1CC[C@H](Nc2ccc(C3CCN(CC(=O)N4CC[C@H](Oc5ccc(-c6cc(F)c7c(c6)C(=O)N(C(C(=O)Nc6nccs6)c6ncn8c6CCC8)C7)cc5)C4)CC3)cc2)C(=O)N1] | *** | | |
| | 2-(6,7-dihydro-5H-pyrrolo[1,2-c]imidazol-1-yl)-2-[6-[4-[(3S)- | | | |
| | 1-[2-[4-[4-[[(3S)-2,6-dioxo-3-piperidyl]amino]phenyl]-1- | | | |
| | piperidyl]acetyl]pyrrolidin-3-yl]oxyphenyl]-4-fluoro-1-oxo- | | | |
| | isoindolin-2-yl]-N-thiazol-2-yl-acetamide | | | |
| 43 | 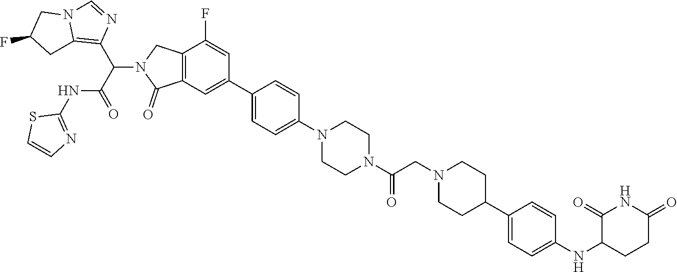 [SMILES: O=C1CCC(Nc2ccc(C3CCN(CC(=O)N4CCN(c5ccc(-c6cc(F)c7c(c6)C(=O)N(C(C(=O)Nc6nccs6)c6ncn8c6C[C@@H](F)C8)C7)cc5)CC4)CC3)cc2)C(=O)N1] | *** | *** | *** |
| | 2-(6-(4-(4-(2-(4-(4-((2,6-dioxopiperidin-3- | | | |
| | yl)amino)phenyl)piperidin-1-yl)acetyl)piperazin-1-yl)phenyl)- | | | |
| | 4-fluoro-1-oxoisoindolin-2-yl)-2-((R)-6-fluoro-6,7-dihydro- | | | |
| | 5H-pyrrolo[1,2-c]imidazol-1-yl)-N-(thiazol-2-yl)acetamide | | | |
| 44 | 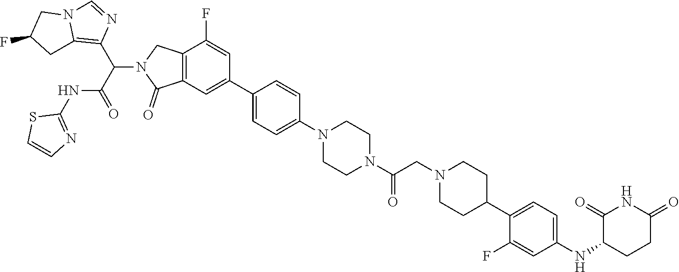 [SMILES: O=C1CC[C@H](Nc2ccc(C3CCN(CC(=O)N4CCN(c5ccc(-c6cc(F)c7c(c6)C(=O)N(C(C(=O)Nc6nccs6)c6ncn8c6C[C@@H](F)C8)C7)cc5)CC4)CC3)c(F)c2)C(=O)N1] | *** | *** | *** |
| | 2-[6-[4-[4-[2-[4-[4-[[(3S)-2,6-dioxo-3-piperidyl]amino]-2- | | | |
| | fluoro-phenyl]-1-piperidyl]acetyl]piperazin-1-yl]phenyl]-4- | | | |
| | fluoro-1-oxo-isoindolin-2-yl]-2-[(6R)-6-fluoro-6,7-dihydro- | | | |
| | 5H-pyrrolo[1,2-c]imidazol-1-yl]-N-thiazol-2-yl-acetamide | | | |
| 45 | 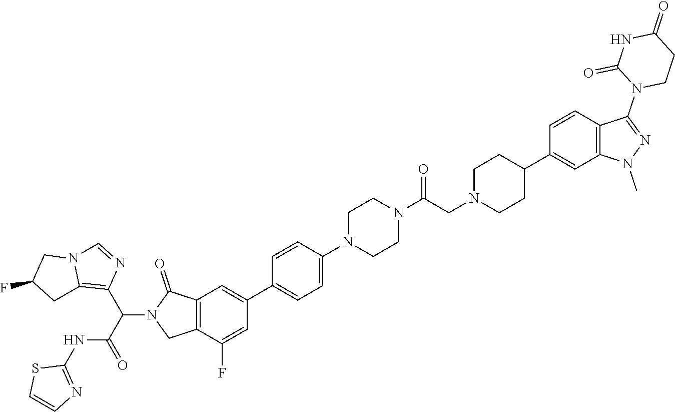 [SMILES: Cn1nc(N2CCC(=O)NC2=O)c2ccc(C3CCN(CC(=O)N4CCN(c5ccc(-c6cc(F)c7c(c6)C(=O)N(C(C(=O)Nc6nccs6)c6ncn8c6C[C@@H](F)C8)C7)cc5)CC4)CC3)cc21] | *** | *** | |
| | 2-[6-[4-[4-[2-[4-[3-(2,4-dioxohexahydropyrimidin-1-yl)-1- | | | |
| | methyl-indazol-6-yl]-1-piperidyl]acetyl]piperazin-1- | | | |
| | yl]phenyl]-4-fluoro-1-oxo-isoindolin-2-yl]-2-[(6R)-6-fluoro- | | | |
| | 6,7-dihydro-5H-pyrrolo[1,2-c]imidazol-1-yl]-N-thiazol-2-yl- | | | |
| | acetamide | | | |
| 46 | 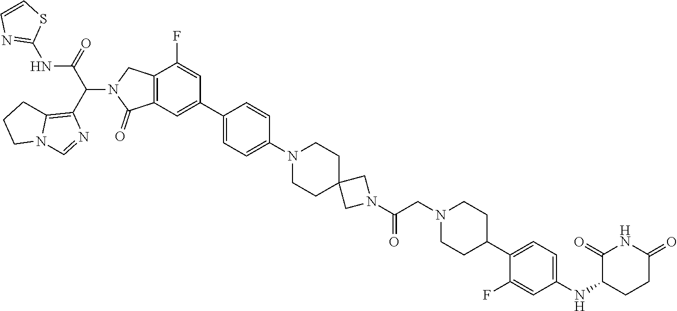 [SMILES: O=C1CC[C@H](Nc2ccc(C3CCN(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1] | ** | ** | |
| | 2-(6,7-dihydro-5H-pyrrolo[1,2-c]imidazol-1-yl)-2-(6-(4-(2-(2- | | | |
| | (4-(4-(((S)-2,6-dioxopiperidin-3-yl)amino)-2- | | | |
| | fluorophenyl)piperidin-1-yl)acetyl)-2,7-diazaspiro[3.5]nonan- | | | |
| | 7-yl)phenyl)-4-fluoro-1-oxoisoindolin-2-yl)-N-(thiazol-2- | | | |
| | yl)acetamide | | | |
| 47 | 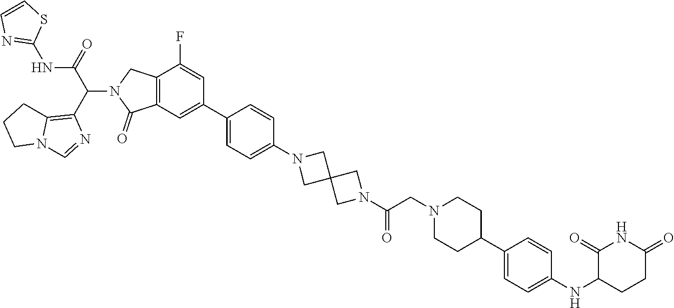 [SMILES: O=C1CCC(Nc2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)cc2)C(=O)N1] | *** | *** | *** |
| | 2-(6,7-dihydro-5H-pyrrolo[1,2-c]imidazol-1-yl)-2-[6-[4-[2-[2- | | | |
| | [4-[4-[(2,6-dioxo-3-piperidyl)amino]phenyl]-1- | | | |
| | piperidyl]acetyl]-2,6-diazaspiro[3.3]heptan-6-yl]phenyl]-4- | | | |
| | fluoro-1-oxo-isoindolin-2-yl]-N-thiazol-2-yl-acetamide | | | |
| 48 | 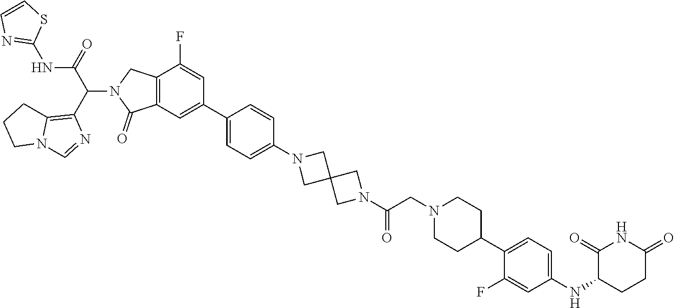 [SMILES: O=C1CC[C@H](Nc2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | *** | *** |
| | 2-(6,7-dihydro-5H-pyrrolo[1,2-c]imidazol-1-yl)-2-[6-[4-[2-[2- | | | |
| | [4-[4-[[(3S)-2,6-dioxo-3-piperidyl]amino]-2-fluoro-phenyl]-1- | | | |
| | piperidyl]acetyl]-2,6-diazaspiro[3.3]heptan-6-yl]phenyl]-4- | | | |
| | fluoro-1-oxo-isoindolin-2-yl]-N-thiazol-2-yl-acetamide | | | |
| 49 | 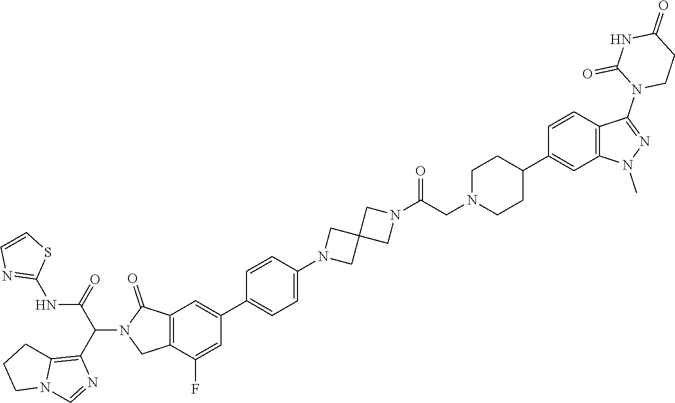 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)cc21] | *** | *** | |
| | 2-(6,7-dihydro-5H-pyrrolo[1,2-c]imidazol-1-yl)-2-[6-[4-[2-[2- | | | |
| | [4-[3-(2,4-dioxohexahydropyrimidin-1-yl)-1-methyl-indazol- | | | |
| | 6-yl]-1-piperidyl]acetyl]-2,6-diazaspiro[3.3]heptan-6- | | | |
| | yl]phenyl]-4-fluoro-1-oxo-isoindolin-2-yl]-N-thiazol-2-yl- | | | |
| | acetamide | | | |
| 51 | 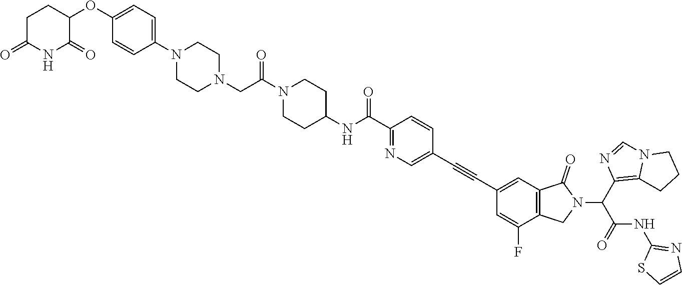 [SMILES: O=C1CCC(Oc2ccc(N3CCN(CC(=O)N4CCC(NC(=O)c5ccc(C#Cc6cc(F)c7c(c6)C(=O)N(C(C(=O)Nc6nccs6)c6ncn8c6CCC8)C7)cn5)CC4)CC3)cc2)C(=O)N1] | *** | | |
| | 5-[2-[2-[1-(6,7-dihydro-5H-pyrrolo[1,2-c]imidazol-1-yl)-2- | | | |
| | oxo-2-(thiazol-2-ylamino)ethyl]-7-fluoro-3-oxo-isoindolin-5- | | | |
| | yl]ethynyl]-N-[1-[2-[4-[4-[(2,6-dioxo-3- | | | |
| | piperidyl)oxy]phenyl]piperazin-1-yl]acetyl]-4- | | | |
| | piperidyl]pyridine-2-carboxamide | | | |
| 52 | 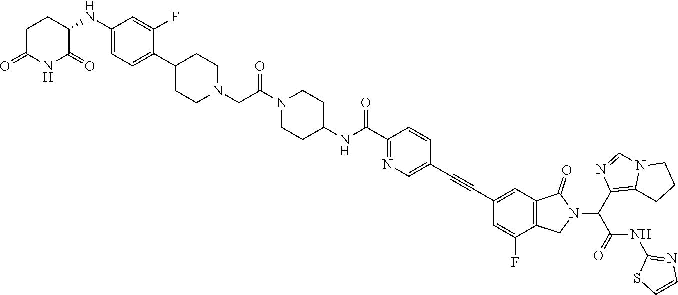 [SMILES: O=C1CC[C@H](Nc2ccc(C3CCN(CC(=O)N4CCC(NC(=O)c5ccc(C#Cc6cc(F)c7c(c6)C(=O)N(C(C(=O)Nc6nccs6)c6ncn8c6CCC8)C7)cn5)CC4)CC3)c(F)c2)C(=O)N1] | *** | *** | |
| | 5-[2-[2-[1-(6,7-dihydro-5H-pyrrolo[1,2-c]imidazol-1-yl)-2- | | | |
| | oxo-2-(thiazol-2-ylamino)ethyl]-7-fluoro-3-oxo-isoindolin-5- | | | |
| | yl]ethynyl]-N-[1-[2-[4-[4-[(3S)-2,6-dioxo-3- | | | |
| | piperidyl]amino]-2-fluoro-phenyl]-1-piperidyl]acetyl]-4- | | | |
| | piperidyllpyridine-2-carboxamide | | | |
| 52 | 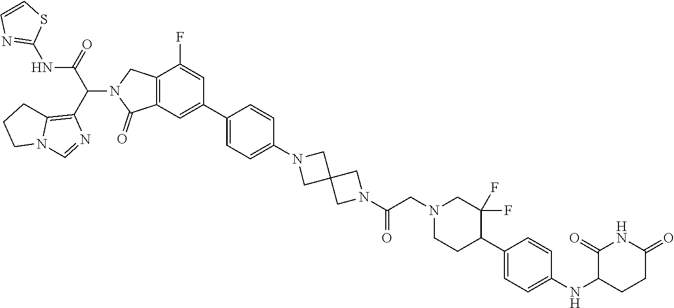 [SMILES: O=C1CCC(Nc2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc2)C(=O)N1] | *** | | |
| | 2-(6,7-dihydro-5H-pyrrolo[1,2-c]imidazol-1-yl)-2-(6-(4-(6-(2- | | | |
| | (4-(4-((2,6-dioxopiperidin-3-yl)amino)phenyl)-3,3- | | | |
| | difluoropiperidin-1-yl)acetyl)-2,6-diazaspiro[3.3]heptan-2- | | | |
| | yl)phenyl)-4-fluoro-1-oxoisoindolin-2-yl)-N-(thiazol-2- | | | |
| | yl)acetamide, Isomer 2 | | | |
| 53 | 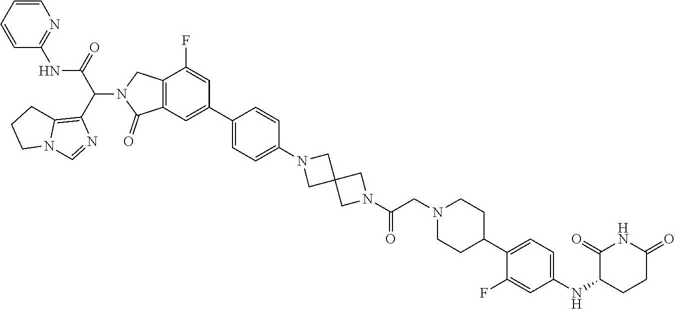 [SMILES: O=C1CC[C@H](Nc2ccc(C3CCN(CC(=O)N4CC5(C4)CN(c4ccc(-c6cc(F)c7c(c6)C(=O)N(C(C(=O)Nc6ccccn6)c6ncn8c6CCC8)C7)cc4)C5)CC3)c(F)c2)C(=O)N1] | ** | *** | |
| | 2-(6,7-dihydro-5H-pyrrolo[1,2-c]imidazol-1-yl)-2-[6-[4-[2-[2- | | | |
| | [4-[4-[[(3S)-2,6-dioxo-3-piperidyl]amino]-2-fluoro-phenyl]-1- | | | |
| | piperidyl]acetyl]-2,6-diazaspiro[3.3]heptan-6-yl]phenyl]-4- | | | |
| | fluoro-1-oxo-isoindolin-2-yl]-N-(2-pyridyl)acetamide | | | |
| 54 | 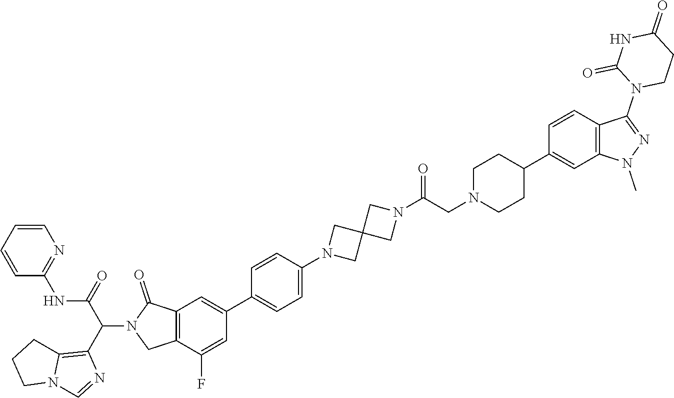 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6ccccn6)c6ncn8c6CCC8)C7)cc4)C5)CC3)cc21] | *** | | |
| | 2-(6,7-dihydro-5H-pyrrolo[1,2-c]imidazol-1-yl)-2-[6-[4-[2-[2- | | | |
| | [4-[3-(2,4-dioxohexahydropyrimidin-1-yl)-1-methyl-indazol- | | | |
| | 6-yl]-1-piperidyl]acetyl]-2,6-diazaspiro[3.3]heptan-6- | | | |
| | yl]phenyl]-4-fluoro-1-oxo-isoindolin-2-yl]-N-(2- | | | |
| | pyridyl)acetamide | | | |
| 55 | 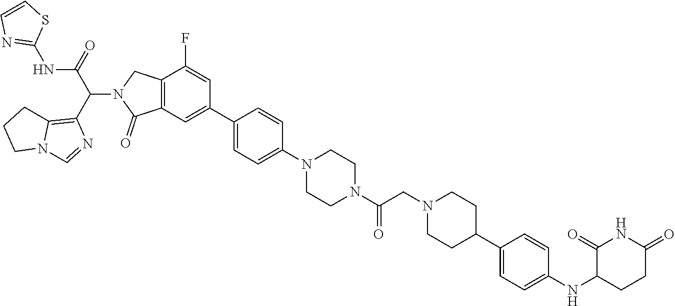 [SMILES: O=C1CCC(Nc2ccc(C3CCN(CC(=O)N4CCN(c5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | *** |
| | 2-(6,7-dihydro-5H-pyrrolo[1,2-c]imidazol-1-yl)-2-[6-[4-[4-[2- | | | |
| | [4-[4-[(2,6-dioxo-3-piperidyl)amino]phenyl]-1- | | | |
| | piperidyl]acetyl]piperazin-1-yl]phenyl]-4-fluoro-1-oxo- | | | |
| | isoindolin-2-yl]-N-thiazol-2-yl-acetamide | | | |
| 56 | 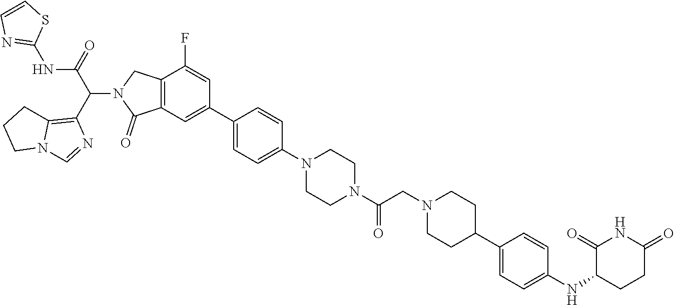 [SMILES: O=C1CC[C@H](Nc2ccc(C3CCN(CC(=O)N4CCN(c5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | *** |
| | 2-(6,7-dihydro-5H-pyrrolo[1,2-c]imidazol-1-yl)-2-[6-[4-[4-[2- | | | |
| | [4-[4-[[(3S)-2,6-dioxo-3-piperidyl]amino]phenyl]-1- | | | |
| | piperidyl]acetyl]piperazin-1-yl]phenyl]-4-fluoro-1-oxo- | | | |
| | isoindolin-2-yl]-N-thiazol-2-yl-acetamide | | | |
| 57 | 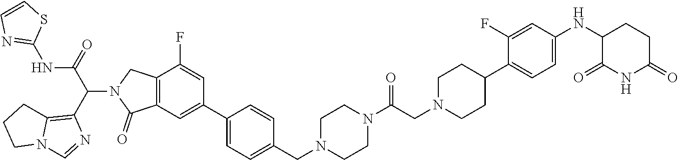 [SMILES: O=C1CCC(Nc2ccc(C3CCN(CC(=O)N4CCN(Cc5ccc(-c6cc(F)c7c(c6)C(=O)N(C(C(=O)Nc6nccs6)c6ncn8c6CCC8)C7)cc5)CC4)CC3)c(F)c2)C(=O)N1] | *** | *** | *** |
| | 2-(6,7-dihydro-5H-pyrrolo[1,2-c]imidazol-1-yl)-2-[6-[4-[[4- | | | |
| | [2-[4-[4-[(2,6-dioxo-3-piperidyl)amino]-2-fluoro-phenyl]-1- | | | |
| | piperidyl]acetyl]piperazin-1-yl]methyl]phenyl]-4-fluoro-1- | | | |
| | oxo-isoindolin-2-yl]-N-thiazol-2-yl-acetamide | | | |
| 58 | 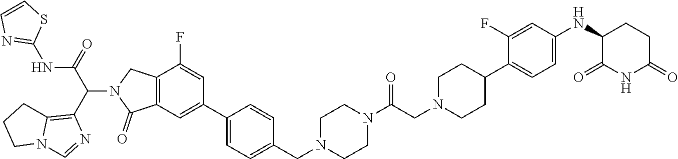 [SMILES: O=C1CC[C@H](Nc2ccc(C3CCN(CC(=O)N4CCN(Cc5ccc(-c6cc(F)c7c(c6)C(=O)N(C(C(=O)Nc6nccs6)c6ncn8c6CCC8)C7)cc5)CC4)CC3)c(F)c2)C(=O)N1] | *** | *** | *** |
| | 2-(6,7-dihydro-5H-pyrrolo[1,2-c]imidazol-1-yl)-2-[6-[4-[[4- | | | |
| | [2-[4-[4-[[(3S)-2,6-dioxo-3-piperidyl]amino]-2-fluoro- | | | |
| | phenyl]-1-piperidyl]acetyl]piperazin-1-yl]methyl]phenyl]-4- | | | |
| | fluoro-1-oxo-isoindolin-2-yl]-N-thiazol-2-yl-acetamide | | | |
| 59 | 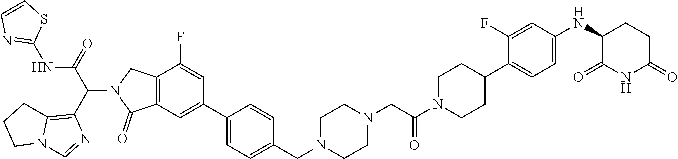 [SMILES: O=C1CC[C@H](Nc2ccc(C3CCN(C(=O)CN4CCN(Cc5ccc(-c6cc(F)c7c(c6)C(=O)N(C(C(=O)Nc6nccs6)c6ncn8c6CCC8)C7)cc5)CC4)CC3)c(F)c2)C(=O)N1] | *** | *** | *** |
| | 2-(6,7-dihydro-5H-pyrrolo[1,2-c]imidazol-1-yl)-2-[6-[4-[[4- | | | |
| | [2-[4-[4-[[(3S)-2,6-dioxo-3-piperidyl]amino]-2-fluoro- | | | |
| | phenyl]-1-piperidyl]-2-oxo-ethyl]piperazin-1- | | | |
| | yl]methyl]phenyl]-4-fluoro-1-oxo-isoindolin-2-yl]-N-thiazol- | | | |
| | 2-yl-acetamide | | | |
| 60 | 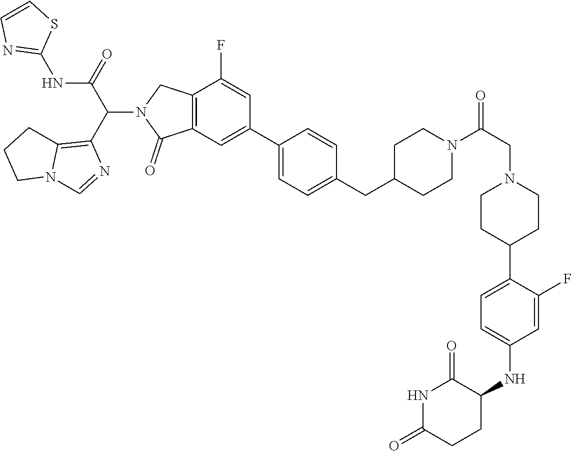 [SMILES: O=C1CC[C@H](Nc2ccc(C3CCN(CC(=O)N4CCC(Cc5ccc(-c6cc(F)c7c(c6)C(=O)N(C(C(=O)Nc6nccs6)c6ncn8c6CCC8)C7)cc5)CC4)CC3)c(F)c2)C(=O)N1] | ** | *** | |
| | 2-(6,7-dihydro-5H-pyrrolo[1,2-c]imidazol-1-yl)-2-[6-[4-[[1- | | | |
| | [2-[4-[4-[[(3S)-2,6-dioxo-3-piperidyl]amino]-2-fluoro- | | | |
| | phenyl]-1-piperidyl]acetyl]-4-piperidyl]methyl]phenyl]-4- | | | |
| | fluoro-1-oxo-isoindolin-2-yl]-N-thiazol-2-yl-acetamide | | | |
| 61 | 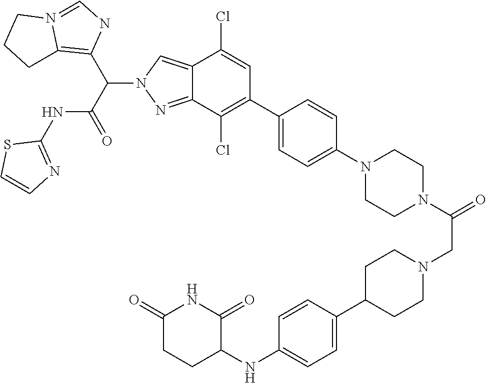 | ** | | |
| | 2-[4,7-dichloro-6-[4-[4-[2-[4-[4-[(2,6-dioxo-3- | | | |
| | piperidyl)amino]phenyl]-1-piperidyl]acetyl]piperazin-1- | | | |
| | yl]phenyl]indazol-2-yl]-2-(6,7-dihydro-5H-pyrrolo[1,2- | | | |
| | c]imidazol-1-yl)-N-thiazol-2-yl-acetamide | | | |
| 62 | 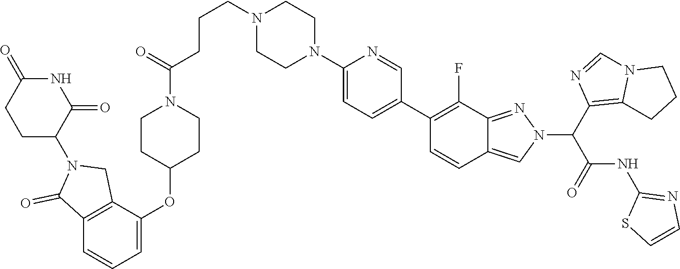 [SMILES: O=C1CCC(N2Cc3c(OC4CCN(C(=O)CCCN5CCN(c6ccc(-c7ccc8cn(C(C(=O)Nc9nccs9)c9ncn%10c9CCC%10)nc8c7F)cn6)CC5)CC4)cccc3C2=O)C(=O)N1] | *** | | |
| | 2-(6,7-dihydro-5H-pyrrolo[1,2-c]imidazol-1-yl)-2-(6-(6-(4-(4- | | | |
| | (4-((2-(2,6-dioxopiperidin-3-yl)-1-oxoisoindolin-4- | | | |
| | yl)oxy)piperidin-1-yl)-4-oxobutyl)piperazin-1-yl)pyridin-3- | | | |
| | yl)-7-fluoro-2H-indazol-2-yl)-N-(thiazol-2-yl)acetamide | | | |
| 63 | 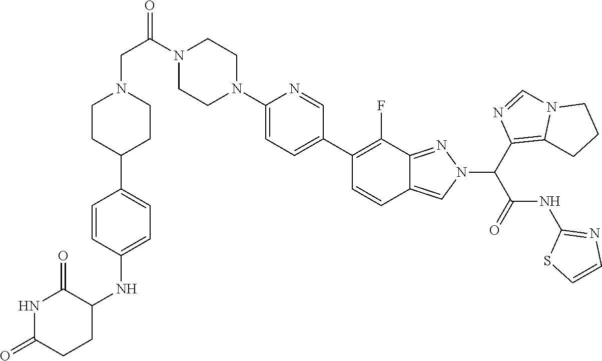 [SMILES: O=C1CCC(Nc2ccc(C3CCN(CC(=O)N4CCN(c5ccc(-c6ccc7cn(C(C(=O)Nc8nccs8)c8ncn9c8CCC9)nc7c6F)cn5)CC4)CC3)cc2)C(=O)N1] | ** | | |
| | 2-(6,7-dihydro-5H-pyrrolo[1,2-c]imidazol-1-yl)-2-(6-(6-(4-(2- | | | |
| | (4-(4-((2,6-dioxopiperidin-3-yl)amino)phenyl)piperidin-1- | | | |
| | yl)acetyl)piperazin-1-yl)pyridin-3-yl)-7-fluoro-2H-indazol-2- | | | |
| | yl)-N-(thiazol-2-yl)acetamide | | | |
| 64 | 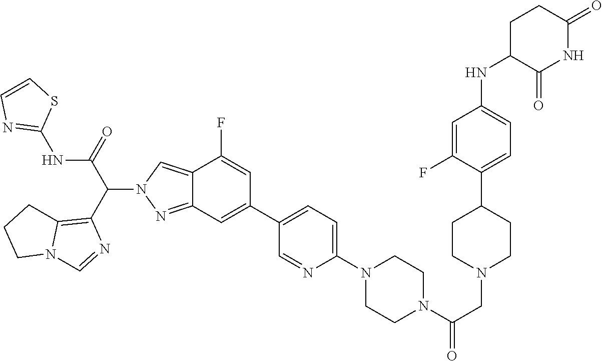 [SMILES: O=C1CCC(Nc2ccc(C3CCN(CC(=O)N4CCN(c5ccc(-c6cc(F)c7cn(C(C(=O)Nc8nccs8)c8ncn9c8CCC9)nc7c6)cn5)CC4)CC3)c(F)c2)C(=O)N1] | ** | | |
| | 2-(6,7-dihydro-5H-pyrrolo[1,2-c]imidazol-1-yl)-2-[6-[6-[4-[2- | | | |
| | [4-[4-[(2,6-dioxo-3-piperidyl)amino]-2-fluoro-phenyl]-1- | | | |
| | piperidyl]acetyl]piperazin-1-yl]-3-pyridyl]-4-fluoro-indazol-2- | | | |
| | yl]-N-thiazol-2-yl-acetamide | | | |
| 65 | 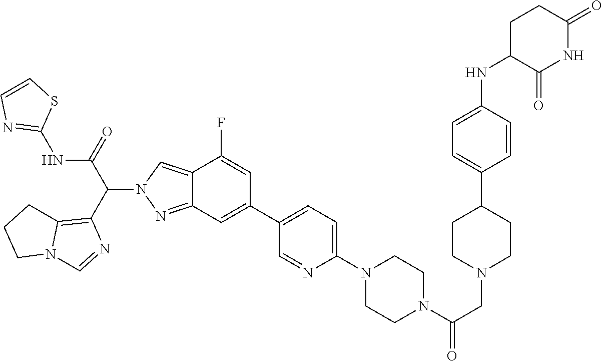 [SMILES: O=C1CCC(Nc2ccc(C3CCN(CC(=O)N4CCN(c5ccc(-c6cc(F)c7cn(C(C(=O)Nc8nccs8)c8ncn9c8CCC9)nc7c6)cn5)CC4)CC3)cc2)C(=O)N1] | *** | | |
| | 2-(6,7-dihydro-5H-pyrrolo[1,2-c]imidazol-1-yl)-2-[6-[6-[4-[2- | | | |
| | [4-[4-[(2,6-dioxo-3-piperidyl)amino]phenyl]-1- | | | |
| | piperidyl]acetyl]piperazin-1-yl]-3-pyridyl]-4-fluoro-indazol-2- | | | |
| | yl]-N-thiazol-2-yl-acetamide | | | |
| 66 | 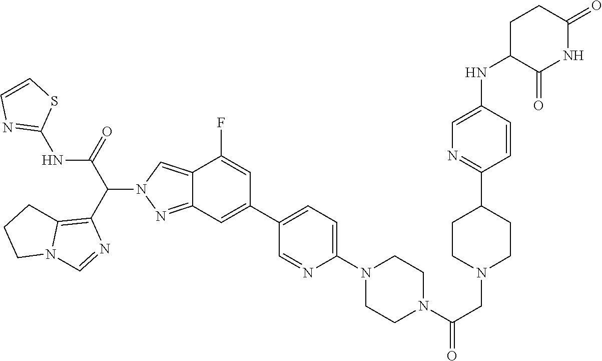 [SMILES: O=C1CCC(Nc2ccc(C3CCN(CC(=O)N4CCN(c5ccc(-c6cc(F)c7cn(C(C(=O)Nc8nccs8)c8ncn9c8CCC9)nc7c6)cn5)CC4)CC3)nc2)C(=O)N1] | *** | *** | |
| | 2-(6,7-dihydro-5H-pyrrolo[1,2-c]imidazol-1-yl)-2-[6-[6-[4-[2- | | | |
| | [4-[5-[(2,6-dioxo-3-piperidyl)amino]-2-pyridyl]-1- | | | |
| | piperidyl]acetyl]piperazin-1-yl]-3-pyridyl]-4-fluoro-indazol-2- | | | |
| | yl]-N-thiazol-2-yl-acetamide | | | |
| 67 | 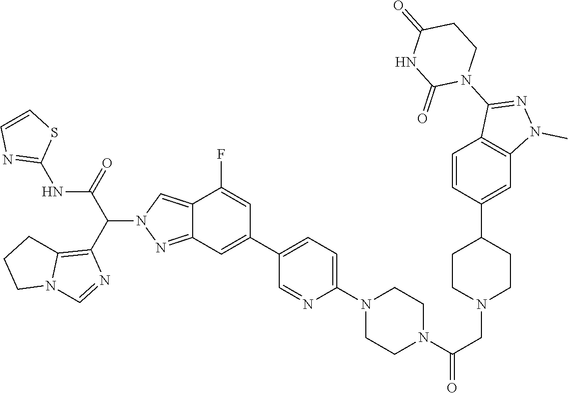 [SMILES: Cn1nc(N2CCC(=O)NC2=O)c2ccc(C3CCN(CC(=O)N4CCN(c5ccc(-c6cc(F)c7cn(C(C(=O)Nc8nccs8)c8ncn9c8CCC9)nc7c6)cn5)CC4)CC3)cc21] | *** | ** | |
| | 2-(6,7-dihydro-5H-pyrrolo[1,2-c]imidazol-1-yl)-2-[6-[6-[4-[2- | | | |
| | [4-[3-(2,4-dioxohexahydropyrimidin-1-yl)-1-methyl-indazol- | | | |
| | 6-yl]-1-piperidyl]acetyl]piperazin-1-yl]-3-pyridyl]-4-fluoro- | | | |
| | indazol-2-yl]-N-thiazol-2-yl-acetamide | | | |
| 68 | 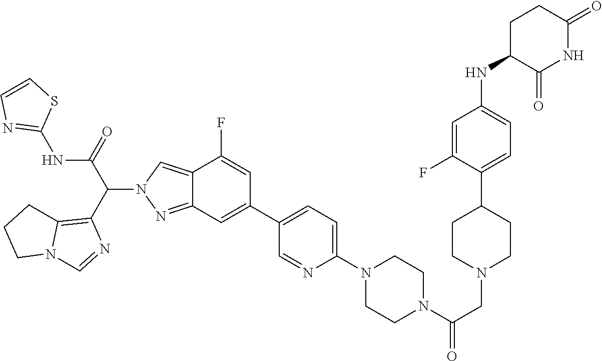 [SMILES: O=C1CC[C@H](Nc2ccc(C3CCN(CC(=O)N4CCN(c5ccc(-c6cc(F)c7cn(C(C(=O)Nc8nccs8)c8ncn9c8CCC9)nc7c6)cn5)CC4)CC3)c(F)c2)C(=O)N1] | *** | *** | |
| | 2-(6,7-dihydro-5H-pyrrolo[1,2-c]imidazol-1-yl)-2-(6-(6-(4-(2- | | | |
| | (4-(4-(((S)-2,6-dioxopiperidin-3-yl)amino)-2- | | | |
| | fluorophenyl)piperidin-1-yl)acetyl)piperazin-1-yl)pyridin-3- | | | |
| | yl)-4-fluoro-2H-indazol-2-yl)-N-(thiazol-2-yl)acetamide | | | |

TABLE 9B

| | | | | H1975 |
|---|---|---|---|---|
| | | | | C797S |
| | | H1975 | H3255 | clone |
| | | EGFR | EGFR | EGFR |
| | | DC50 | DC50 | DC50 |
| Compound | | [nM] | [nM] | [nM] |
| 69 | 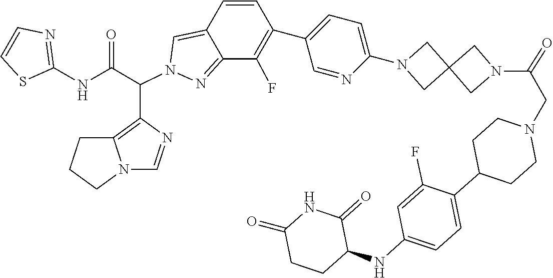 [SMILES: O=C1CC[C@H](Nc2ccc(C3CCN(CC(=O)N4CC5(C4)CN(c4ccc(-c6ccc7cn(C(C(=O)Nc8nccs8)c8ncn9c8CCC9)nc7c6F)cn4)C5)CC3)c(F)c2)C(=O)N1] | *** | | *** |
| | 2-(6,7-dihydro-5H-pyrrolo[1,2-c]imidazol-1-yl)-2-(6-(6-(6- | | | |
| | (2-(4-(4-(((S)-2,6-dioxopiperidin-3-yl)amino)-2- | | | |
| | fluorophenyl)piperidin-1-yl)acetyl)-2,6- | | | |
| | diazaspiro[3.3]heptan-2-yl)pyridin-3-yl)-7-fluoro-2H- | | | |
| | indazol-2-yl)-N-(thiazol-2-yl)acetamide | | | |
| 70 | 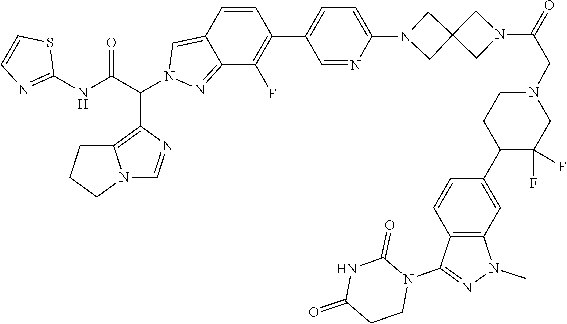 [SMILES: Cn1nc(N2CCC(=O)NC2=O)c2ccc(C3CCN(CC(=O)N4CC5(C4)CN(c4ccc(-c6ccc7cn(C(C(=O)Nc8nccs8)c8ncn9c8CCC9)nc7c6F)cn4)C5)CC3(F)F)cc21] | *** | | *** |
| | 2-(6,7-dihydro-5H-pyrrolo[1,2-c]imidazol-1-yl)-2-(6-(6-(6- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)acetyl)-2,6- | | | |
| | diazaspiro[3.3]heptan-2-yl)pyridin-3-yl)-7-fluoro-2H- | | | |
| | indazol-2-yl)-N-(thiazol-2-yl)acetamide | | | |
| 71 | 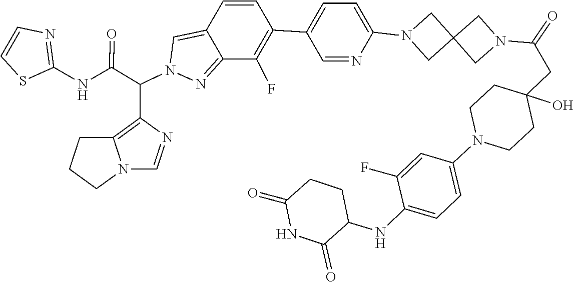 [SMILES: O=C1CCC(Nc2ccc(N3CCC(O)(CC(=O)N4CC5(C4)CN(c4ccc(-c6ccc7cn(C(C(=O)Nc8nccs8)c8ncn9c8CCC9)nc7c6F)cn4)C5)CC3)cc2F)C(=O)N1] | *** | | |
| | 2-(6,7-dihydro-5H-pyrrolo[1,2-c]imidazol-1-yl)-2-(6-(6-(6- | | | |
| | (2-(1-(4-((2,6-dioxopiperidin-3-yl)amino)-3-fluorophenyl)-4- | | | |
| | hydroxypiperidin-4-yl)acetyl)-2,6-diazaspiro[3.3]heptan-2- | | | |
| | yl)pyridin-3-yl)-7-fluoro-2H-indazol-2-yl)-N-(thiazol-2- | | | |
| | yl)acetamide | | | |
| 72 | 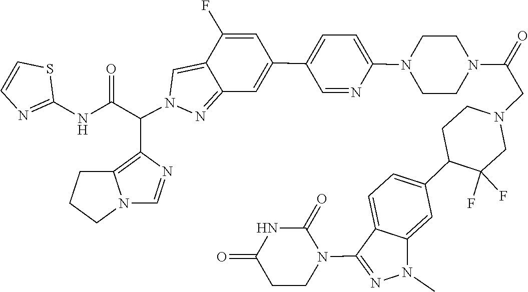 [SMILES: Cn1nc(N2CCC(=O)NC2=O)c2ccc(C3CCN(CC(=O)N4CCN(c5ccc(-c6cc(F)c7cn(C(C(=O)Nc8nccs8)c8ncn9c8CCC9)nc7c6)cn5)CC4)CC3(F)F)cc21] | *** | | |
| | 2-(6,7-dihydro-5H-pyrrolo[1,2-c]imidazol-1-yl)-2-(6-(6-(4- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)acetyl)piperazin- | | | |
| | 1-yl)pyridin-3-yl)-4-fluoro-2H-indazol-2-yl)-N-(thiazol-2- | | | |
| | yl)acetamide | | | |
| 73 | 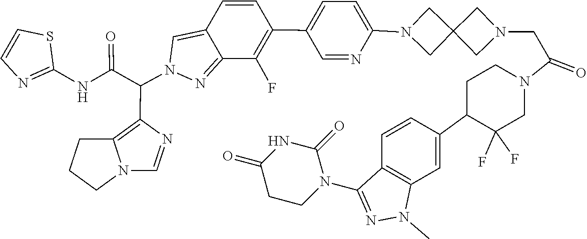 [SMILES: Cn1nc(N2CCC(=O)NC2=O)c2ccc(C3CCN(C(=O)CN4CC5(C4)CN(c4ccc(-c6ccc7cn(C(C(=O)Nc8nccs8)c8ncn9c8CCC9)nc7c6F)cn4)C5)CC3(F)F)cc21] | *** | *** | |
| | 2-(6,7-dihydro-5H-pyrrolo[1,2-c]imidazol-1-yl)-2-(6-(6-(6- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)-2-oxoethyl)- | | | |
| | 2,6-diazaspiro[3.3]heptan-2-yl)pyridin-3-yl)-7-fluoro-2H- | | | |
| | indazol-2-yl)-N-(thiazol-2-yl)acetamide | | | |
| 74 | 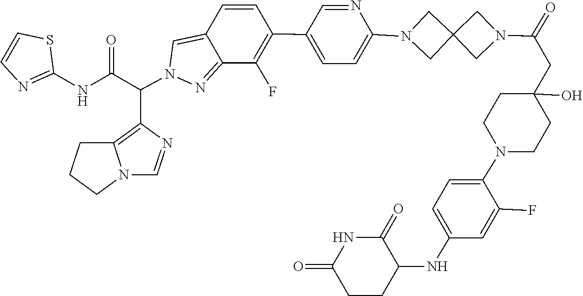 [SMILES: O=C1CCC(Nc2ccc(N3CCC(O)(CC(=O)N4CC5(C4)CN(c4ccc(-c6ccc7cn(C(C(=O)Nc8nccs8)c8ncn9c8CCC9)nc7c6F)cn4)C5)CC3)c(F)c2)C(=O)N1] | *** | | |
| | 2-(6,7-dihydro-5H-pyrrolo[1,2-c]imidazol-1-yl)-2-(6-(6-(6- | | | |
| | (2-(1-(4-((2,6-dioxopiperidin-3-yl)amino)-2-fluorophenyl)-4- | | | |
| | hydroxypiperidin-4-yl)acetyl)-2,6-diazaspiro[3.3]heptan-2- | | | |
| | yl)pyridin-3-yl)-7-fluoro-2H-indazol-2-yl)-N-(thiazol-2- | | | |
| | yl)acetamide | | | |
| 75 | 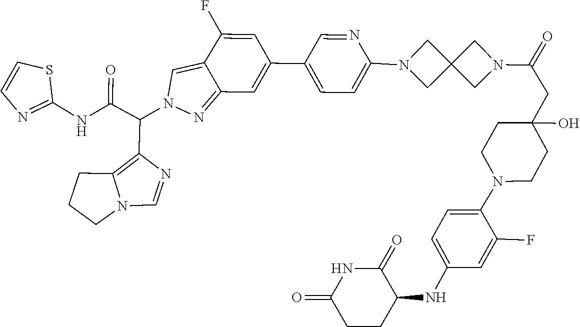 [SMILES: O=C1CC[C@H](Nc2ccc(N3CCC(O)(CC(=O)N4CC5(C4)CN(c4ccc(-c6cc(F)c7cn(C(C(=O)Nc8nccs8)c8ncn9c8CCC9)nc7c6)cn4)C5)CC3)c(F)c2)C(=O)N1] | *** | | |
| | 2-(6,7-dihydro-5H-pyrrolo[1,2-c]imidazol-1-yl)-2-(6-(6-(6- | | | |
| | (2-(1-(4-(((S)-2,6-dioxopiperidin-3-yl)amino)-2- | | | |
| | fluorophenyl)-4-hydroxypiperidin-4-yl)acetyl)-2,6- | | | |
| | diazaspiro[3.3]heptan-2-yl)pyridin-3-yl)-4-fluoro-2H- | | | |
| | indazol-2-yl)-N-(thiazol-2-yl)acetamide | | | |
| 76 | 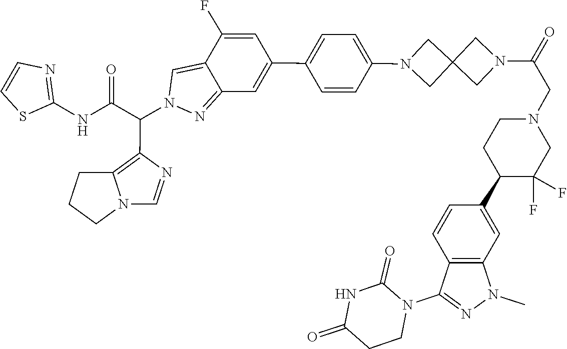 [SMILES: Cn1nc(N2CCC(=O)NC2=O)c2ccc([C@H]3CCN(CC(=O)N4CC5(C4)CN(c4ccc(-c6cc(F)c7cn(C(C(=O)Nc8nccs8)c8ncn9c8CCC9)nc7c6)cc4)C5)CC3(F)F)cc21] | *** | | |
| | 2-(6,7-dihydro-5H-pyrrolo[1,2-c]imidazol-1-yl)-2-(6-(4-(6- | | | |
| | (2-((R)-4-(3-(2,4-dioxotetrahydropyrimidin-1(2H)-yl)-1- | | | |
| | methyl-1H-indazol-6-yl)-3,3-difluoropiperidin-1-yl)acetyl)- | | | |
| | 2,6-diazaspiro[3.3]heptan-2-yl)phenyl)-4-fluoro-2H-indazol- | | | |
| | 2-yl)-N-(thiazol-2-yl)acetamide | | | |
| 77 | 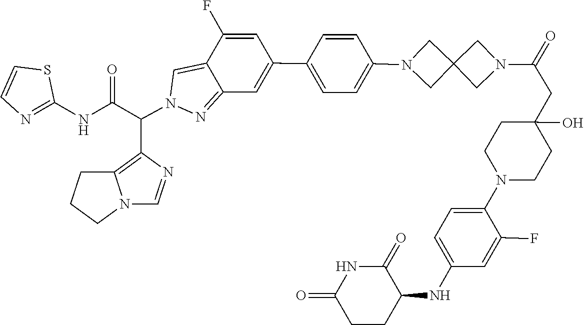 [SMILES: O=C1CC[C@H](Nc2ccc(N3CCC(O)(CC(=O)N4CC5(C4)CN(c4ccc(-c6cc(F)c7cn(C(C(=O)Nc8nccs8)c8ncn9c8CCC9)nc7c6)cc4)C5)CC3)c(F)c2)C(=O)N1] | *** | | |
| | 2-(6,7-dihydro-5H-pyrrolo[1,2-c]imidazol-1-yl)-2-(6-(4-(6- | | | |
| | (2-(1-(4-(((S)-2,6-dioxopiperidin-3-yl)amino)-2- | | | |
| | fluorophenyl)-4-hydroxypiperidin-4-yl)acetyl)-2,6- | | | |
| | diazaspiro[3.3]heptan-2-yl)phenyl)-4-fluoro-2H-indazol-2- | | | |
| | yl)-N-(thiazol-2-yl)acetamide | | | |
| 78 | 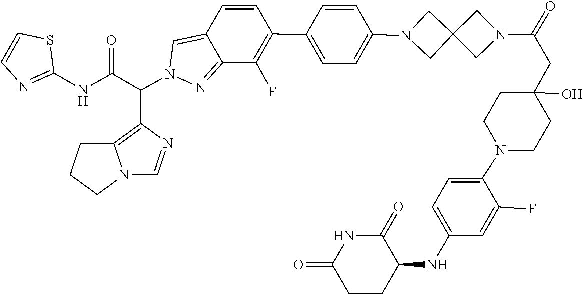 [SMILES: O=C1CC[C@H](Nc2ccc(N3CCC(O)(CC(=O)N4CC5(C4)CN(c4ccc(-c6ccc7cn(C(C(=O)Nc8nccs8)c8ncn9c8CCC9)nc7c6F)cc4)C5)CC3)c(F)c2)C(=O)N1] | *** | | |
| | 2-(6,7-dihydro-5H-pyrrolo[1,2-c]imidazol-1-yl)-2-(6-(4-(6- | | | |
| | (2-(1-(4-(((S)-2,6-dioxopiperidin-3-yl)amino)-2- | | | |
| | fluorophenyl)-4-hydroxypiperidin-4-yl)acetyl)-2,6- | | | |
| | diazaspiro[3.3]heptan-2-yl)phenyl)-7-fluoro-2H-indazol-2- | | | |
| | yl)-N-(thiazol-2-yl)acetamide | | | |
| 79 | 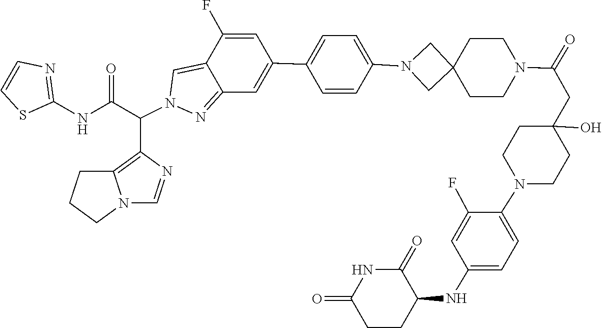 [SMILES: O=C1CC[C@H](Nc2ccc(N3CCC(O)(CC(=O)N4CCC5(CC4)CN(c4ccc(-c6cc(F)c7cn(C(C(=O)Nc8nccs8)c8ncn9c8CCC9)nc7c6)cc4)C5)CC3)c(F)c2)C(=O)N1] | *** | | |
| | 2-(6,7-dihydro-5H-pyrrolo[1,2-c]imidazol-1-yl)-2-(6-(4-(7- | | | |
| | (2-(1-(4-(((S)-2,6-dioxopiperidin-3-yl)amino)-2- | | | |
| | fluorophenyl)-4-hydroxypiperidin-4-yl)acetyl)-2,7- | | | |
| | diazaspiro[3.5]nonan-2-yl)phenyl)-4-fluoro-2H-indazol-2- | | | |
| | yl)-N-(thiazol-2-yl)acetamide | | | |
| 80 | 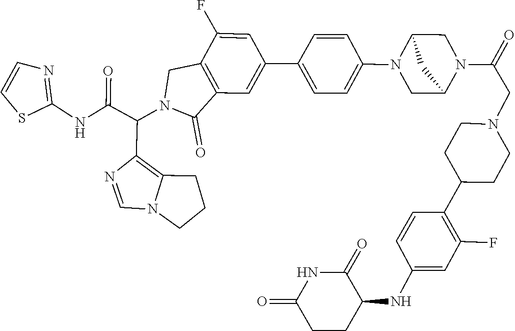 [SMILES: O=C1CC[C@H](Nc2ccc(C3CCN(CC(=O)N4C[C@H]5C[C@@H]4CN5c4ccc(-c5cc(F)c6c(c5)C(=O)N(C(C(=O)Nc5nccs5)c5ncn7c5CCC7)C6)cc4)CC3)c(F)c2)C(=O)N1] | ** | *** | |
| | 2-(6,7-dihydro-5H-pyrrolo[1,2-c]imidazol-1-yl)-2-(6-(4- | | | |
| | ((1R,4R)-5-(2-(4-(4-(((S)-2,6-dioxopiperidin-3-yl)amino)-2- | | | |
| | fluorophenyl)piperidin-1-yl)acetyl)-2,5- | | | |
| | diazabicyclo[2.2.1]heptan-2-yl)phenyl)-4-fluoro-1- | | | |
| | oxoisoindolin-2-yl)-N-(thiazol-2-yl)acetamide | | | |
| 81 | 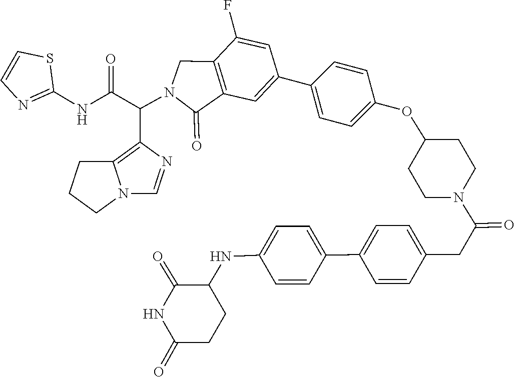 [SMILES: O=C1CCC(Nc2ccc(-c3ccc(CC(=O)N4CCC(Oc5ccc(-c6cc(F)c7c(c6)C(=O)N(C(C(=O)Nc6nccs6)c6ncn8c6CCC8)C7)cc5)CC4)cc3)cc2)C(=O)N1] | ** | | |
| | 2-(6,7-dihydro-5H-pyrrolo[1,2-c]imidazol-1-yl)-2-(6-(4-((1- | | | |
| | (2-(4′-((2,6-dioxopiperidin-3-yl)amino)-[1,1′-biphenyl]-4- | | | |
| | yl)acetyl)piperidin-4-yl)oxy)phenyl)-4-fluoro-1- | | | |
| | oxoisoindolin-2-yl)-N-(thiazol-2-yl)acetamide | | | |
| 82 | 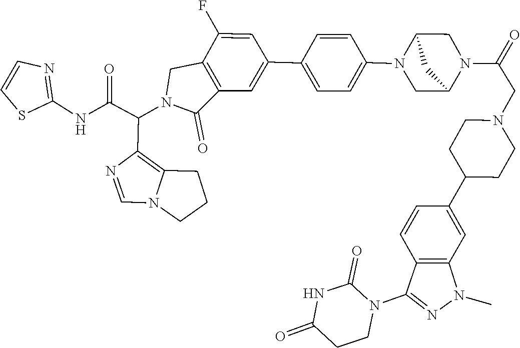 [SMILES: Cn1nc(N2CCC(=O)NC2=O)c2ccc(C3CCN(CC(=O)N4C[C@H]5C[C@@H]4CN5c4ccc(-c5cc(F)c6c(c5)C(=O)N(C(C(=O)Nc5nccs5)c5ncn7c5CCC7)C6)cc4)CC3)cc21] | * | | |
| | 2-(6,7-dihydro-5H-pyrrolo[1,2-c]imidazol-1-yl)-2-(6-(4- | | | |
| | ((1R,4R)-5-(2-(4-(3-(2,4-dioxotetrahydropyrimidin-1(2H)- | | | |
| | yl)-1-methyl-1H-indazol-6-yl)piperidin-1-yl)acetyl)-2,5- | | | |
| | diazabicyclo[2.2.1]heptan-2-yl)phenyl)-4-fluoro-1- | | | |
| | oxoisoindolin-2-yl)-N-(thiazol-2-yl)acetamide | | | |
| 83 | 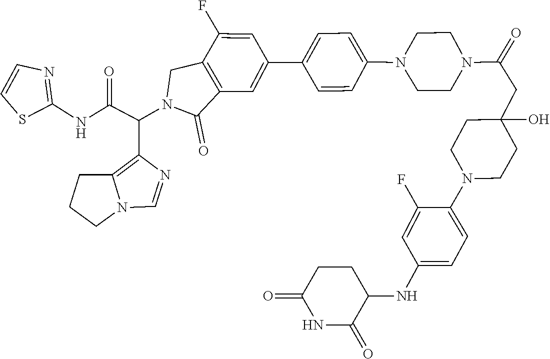 [SMILES: O=C1CCC(Nc2ccc(N3CCC(O)(CC(=O)N4CCN(c5ccc(-c6cc(F)c7c(c6)C(=O)N(C(C(=O)Nc6nccs6)c6ncn8c6CCC8)C7)cc5)CC4)CC3)c(F)c2)C(=O)N1] | *** | *** | |
| | 2-(6,7-dihydro-5H-pyrrolo[1,2-c]imidazol-1-yl)-2-(6-(4-(4- | | | |
| | (2-(1-(4-((2,6-dioxopiperidin-3-yl)amino)-2-fluorophenyl)-4- | | | |
| | hydroxypiperidin-4-yl)acetyl)piperazin-1-yl)phenyl)-4- | | | |
| | fluoro-1-oxoisoindolin-2-yl)-N-(thiazol-2-yl)acetamide | | | |
| 84 | 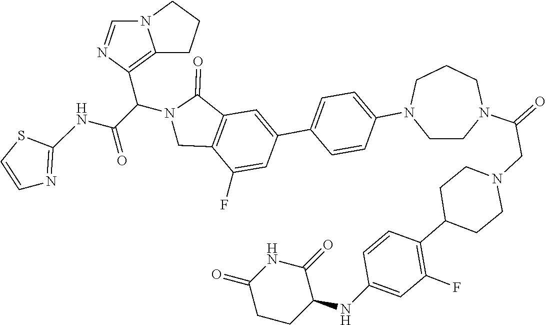 [SMILES: O=C1CC[C@H](Nc2ccc(C3CCN(CC(=O)N4CCCN(c5ccc(-c6cc(F)c7c(c6)C(=O)N(C(C(=O)Nc6nccs6)c6ncn8c6CCC8)C7)cc5)CC4)CC3)c(F)c2)C(=O)N1] | ** | ** | |
| | 2-(6,7-dihydro-5H-pyrrolo[1,2-c]imidazol-1-yl)-2-(6-(4-(4- | | | |
| | (2-(4-(4-(((S)-2,6-dioxopiperidin-3-yl)amino)-2- | | | |
| | fluorophenyl)piperidin-1-yl)acetyl)-1,4-diazepan-1- | | | |
| | yl)phenyl)-4-fluoro-1-oxoisoindolin-2-yl)-N-(thiazol-2- | | | |
| | yl)acetamide | | | |
| 85 | 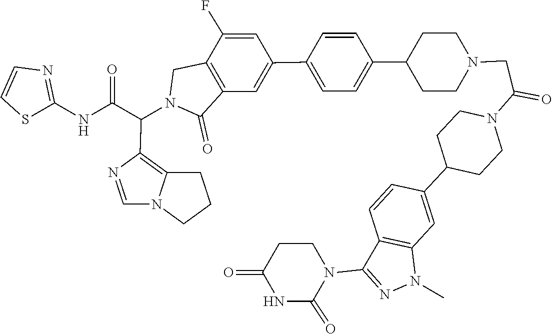 [SMILES: Cn1nc(N2CCC(=O)NC2=O)c2ccc(C3CCN(C(=O)CN4CCC(c5ccc(-c6cc(F)c7c(c6)C(=O)N(C(C(=O)Nc6nccs6)c6ncn8c6CCC8)C7)cc5)CC4)CC3)cc21] | ** | *** | |
| | 2-(6,7-dihydro-5H-pyrrolo[1,2-c]imidazol-1-yl)-2-(6-(4-(1- | | | |
| | (2-(4-(3-(2,4-dioxotetrahydropyrimidin-1(2H)-yl)-1-methyl- | | | |
| | 1H-indazol-6-yl)piperidin-1-yl)-2-oxoethyl)piperidin-4- | | | |
| | yl)phenyl)-4-fluoro-1-oxoisoindolin-2-yl)-N-(thiazol-2- | | | |
| | yl)acetamide | | | |
| 86 | 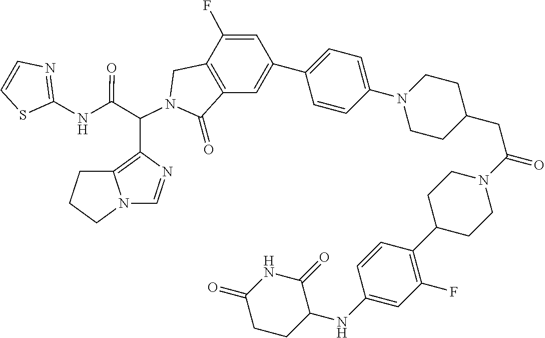 [SMILES: O=C1CCC(Nc2ccc(C3CCN(C(=O)CC4CCN(c5ccc(-c6cc(F)c7c(c6)C(=O)N(C(C(=O)Nc6nccs6)c6ncn8c6CCC8)C7)cc5)CC4)CC3)c(F)c2)C(=O)N1] | ** | | |
| | 2-(6,7-dihydro-5H-pyrrolo[1,2-c]imidazol-1-yl)-2-(6-(4-(4- | | | |
| | (2-(4-(4-((2,6-dioxopiperidin-3-yl)amino)-2- | | | |
| | fluorophenyl)piperidin-1-yl)-2-oxoethyl)piperidin-1- | | | |
| | yl)phenyl)-4-fluoro-1-oxoisoindolin-2-yl)-N-(thiazol-2- | | | |
| | yl)acetamide | | | |
| 87 | 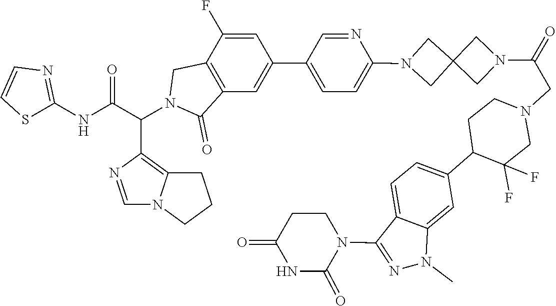 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cn4)C5)CC3(F)F)cc21] | *** | | *** |
| | 2-(6,7-dihydro-5H-pyrrolo[1,2-c]imidazol-1-yl)-2-(6-(6-(6- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)acetyl)-2,6- | | | |
| | diazaspiro[3.3]heptan-2-yl)pyridin-3-yl)-4-fluoro-1- | | | |
| | oxoisoindolin-2-yl)-N-(thiazol-2-yl)acetamide | | | |
| 88 | 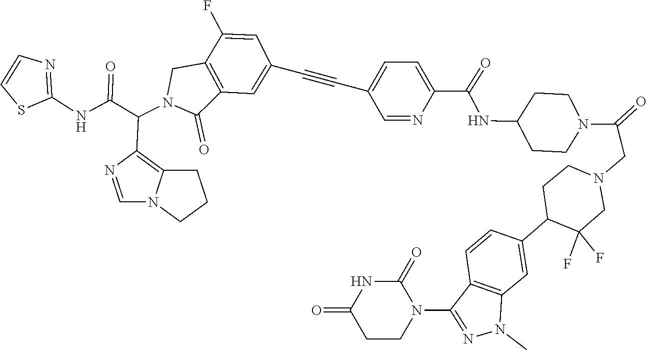 [SMILES: Cn1nc(N2CCC(=O)NC2=O)c2ccc(C3CCN(CC(=O)N4CCC(NC(=O)c5ccc(C#Cc6cc(F)c7c(c6)C(=O)N(C(C(=O)Nc6nccs6)c6ncn8c6CCC8)C7)cn5)CC4)CC3(F)F)cc21] | *** | | *** |
| | 5-((2-(1-(6,7-dihydro-5H-pyrrolo[1,2-c]imidazol-1-yl)-2- | | | |
| | oxo-2-(thiazol-2-ylamino)ethyl)-7-fluoro-3-oxoisoindolin-5- | | | |
| | yl)ethynyl)-N-(1-(2-(4-(3-(2,4-dioxotetrahydropyrimidin- | | | |
| | 1(2H)-yl)-1-methyl-1H-indazol-6-yl)-3,3-difluoropiperidin- | | | |
| | 1-yl)acetyl)piperidin-4-yl)picolinamide | | | |
| 89 | 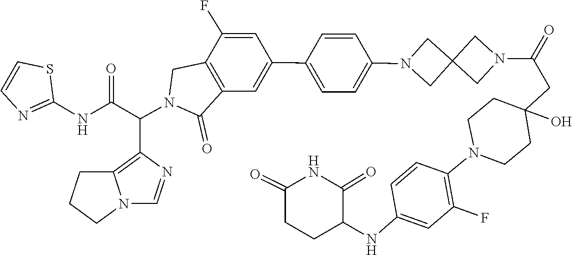 [SMILES: O=C1CCC(Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-(6-(4-(6- | | | |
| | (2-(1-(4-((2,6-dioxopiperidin-3-yl)amino)-2-fluorophenyl)-4- | | | |
| | hydroxypiperidin-4-yl)acetyl)-2,6-diazaspiro[3.3]heptan-2- | | | |
| | yl)phenyl)-4-fluoro-1-oxoisoindolin-2-yl)-N-(thiazol-2- | | | |
| | yl)acetamide | | | |
| 90 | 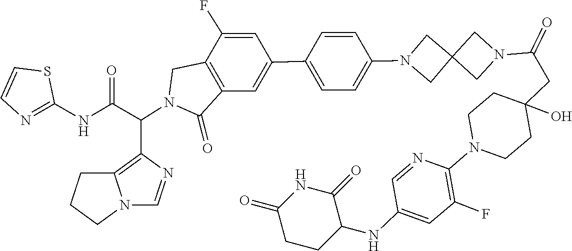 [SMILES: O=C1CCC(Nc2cnc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-(6-(4-(6- | | | |
| | (2-(1-(5-((2,6-dioxopiperidin-3-yl)amino)-3-fluoropyridin-2- | | | |
| | yl)-4-hydroxypiperidin-4-yl)acetyl)-2,6- | | | |
| | diazaspiro[3.3]heptan-2-yl)phenyl)-4-fluoro-1-oxoisoindolin- | | | |
| | 2-yl)-N-(thiazol-2-yl)acetamide | | | |
| 91 | 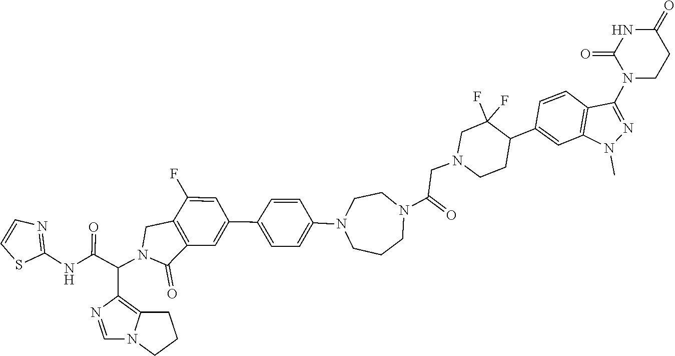 [SMILES: Cn1nc(N2CCC(=O)NC2=O)c2ccc(C3CCN(CC(=O)N4CCCN(c5ccc(-c6cc(F)c7c(c6)C(=O)N(C(C(=O)Nc6nccs6)c6ncn8c6CCC8)C7)cc5)CC4)CC3(F)F)cc21] | *** | | ** |
| | 2-(6,7-dihydro-5H-pyrrolo[1,2-c]imidazol-1-yl)-2-(6-(4-(4- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)acetyl)-1,4- | | | |
| | diazepan-1-yl)phenyl)-4-fluoro-1-oxoisoindolin-2-yl)-N- | | | |
| | (thiazol-2-yl)acetamide | | | |
| 92 | 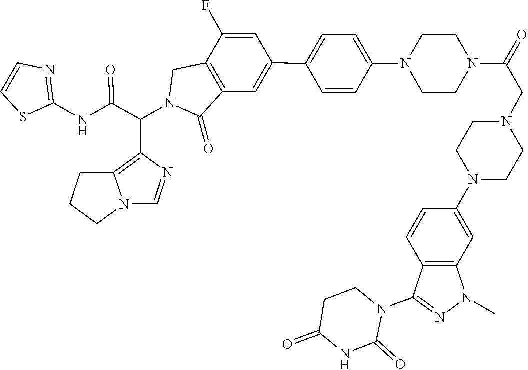 [SMILES: Cn1nc(N2CCC(=O)NC2=O)c2ccc(N3CCN(CC(=O)N4CCN(c5ccc(-c6cc(F)c7c(c6)C(=O)N(C(C(=O)Nc6nccs6)c6ncn8c6CCC8)C7)cc5)CC4)CC3)cc21] | *** | | *** |
| | 2-(6,7-dihydro-5H-pyrrolo[1,2-c]imidazol-1-yl)-2-(6-(4-(4- | | | |
| | (2-(4-(3-(2,4-dioxotetrahydropyrimidin-1(2H)-yl)-1-methyl- | | | |
| | 1H-indazol-6-yl)piperazin-1-yl)acetyl)piperazin-1- | | | |
| | yl)phenyl)-4-fluoro-1-oxoisoindolin-2-yl)-N-(thiazol-2- | | | |
| | yl)acetamide | | | |
| 93 | 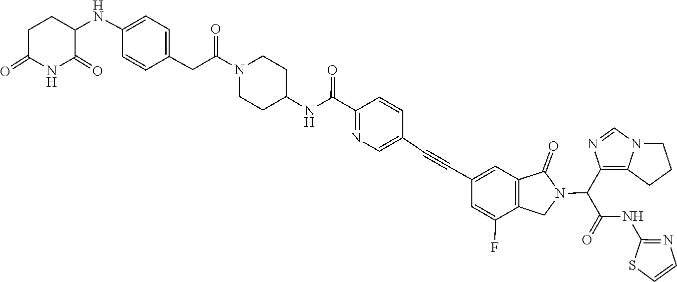 [SMILES: O=C1CCC(Nc2ccc(CC(=O)N3CCC(NC(=O)c4ccc(C#Cc5cc(F)c6c(c5)C(=O)N(C(C(=O)Nc5nccs5)c5ncn7c5CCC7)C6)cn4)CC3)cc2)C(=O)N1] | *** | | |
| | 5-[2-[2-[1-(6,7-dihydro-5H-pyrrolo[1,2-c]imidazol-1-yl)-2- | | | |
| | oxo-2-(thiazol-2-ylamino)ethyl]-7-fluoro-3-oxo-isoindolin-5- | | | |
| | yl]ethynyl]-N-[1-[2-[4-[(2,6-dioxo-3- | | | |
| | piperidyl)amino]phenyl]acetyl]-4-piperidyl]pyridine-2- | | | |
| | carboxamide | | | |
| 94 | 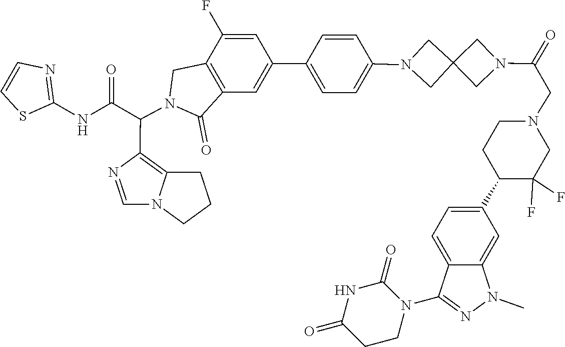 [SMILES: Cn1nc(N2CCC(=O)NC2=O)c2ccc([C@@H]3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21] | *** | *** | *** |
| | 2-(6,7-dihydro-5H-pyrrolo[1,2-c]imidazol-1-yl)-2-(6-(4-(6- | | | |
| | (2-((S)-4-(3-(2,4-dioxotetrahydropyrimidin-1(2H)-yl)-1- | | | |
| | methyl-1H-indazol-6-yl)-3,3-difluoropiperidin-1-yl)acetyl)- | | | |
| | 2,6-diazaspiro[3.3]heptan-2-yl)phenyl)-4-fluoro-1- | | | |
| | oxoisoindolin-2-yl)-N-(thiazol-2-yl)acetamide | | | |
| 95 | 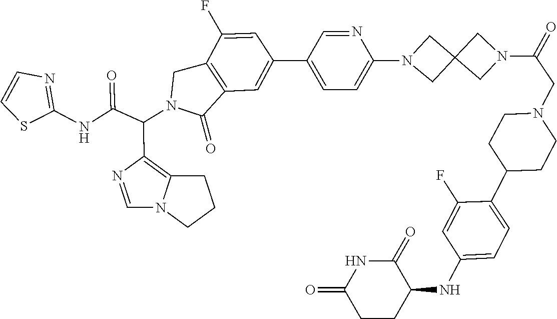 [SMILES: O=C1CC[C@H](Nc2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cn4)C5)CC3)c(F)c2)C(=O)N1] | *** | | |
| | 2-(6,7-dihydro-5H-pyrrolo[1,2-c]imidazol-1-yl)-2-(6-(6-(6- | | | |
| | (2-(4-(4-(((S)-2,6-dioxopiperidin-3-yl)amino)-2- | | | |
| | fluorophenyl)piperidin-1-yl)acetyl)-2,6- | | | |
| | diazaspiro[3.3]heptan-2-yl)pyridin-3-yl)-4-fluoro-1- | | | |
| | oxoisoindolin-2-yl)-N-(thiazol-2-yl)acetamide | | | |
| 96 | 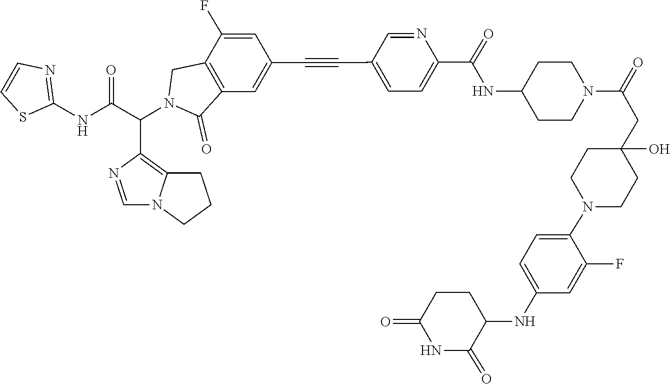 [SMILES: O=C1CCC(Nc2ccc(N3CCC(O)(CC(=O)N4CCC(NC(=O)c5ccc(C#Cc6cc(F)c7c(c6)C(=O)N(C(C(=O)Nc6nccs6)c6ncn8c6CCC8)C7)cn5)CC4)CC3)c(F)c2)C(=O)N1] | *** | | *** |
| | 5-((2-(1-(6,7-dihydro-5H-pyrrolo[1,2-c]imidazol-1-yl)-2- | | | |
| | oxo-2-(thiazol-2-ylamino)ethyl)-7-fluoro-3-oxoisoindolin-5- | | | |
| | yl)ethynyl)-N-(1-(2-(1-(4-((2,6-dioxopiperidin-3-yl)amino)- | | | |
| | 2-fluorophenyl)-4-hydroxypiperidin-4-yl)acetyl)piperidin-4- | | | |
| | yl)picolinamide | | | |
| 97 | 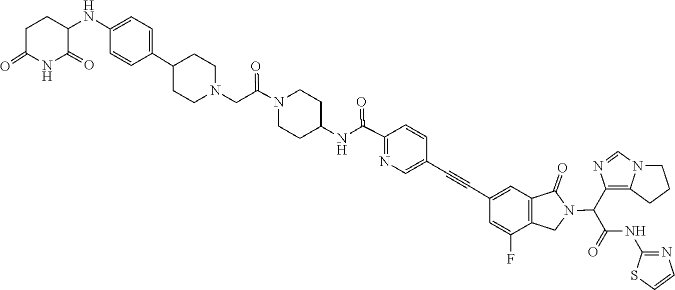 [SMILES: O=C1CCC(Nc2ccc(C3CCN(CC(=O)N4CCC(NC(=O)c5ccc(C#Cc6cc(F)c7c(c6)C(=O)N(C(C(=O)Nc6nccs6)c6ncn8c6CCC8)C7)cn5)CC4)CC3)cc2)C(=O)N1] | *** | *** | *** |
| | 5-((2-(1-(6,7-dihydro-5H-pyrrolo[1,2-c]imidazol-1-yl)-2- | | | |
| | oxo-2-(thiazol-2-ylamino)ethyl)-7-fluoro-3-oxoisoindolin-5- | | | |
| | yl)ethynyl)-N-(1-(2-(4-(4-((2,6-dioxopiperidin-3- | | | |
| | yl)amino)phenyl)piperidin-1-yl)acetyl)piperidin-4- | | | |
| | yl)picolinamide | | | |
| 98 | 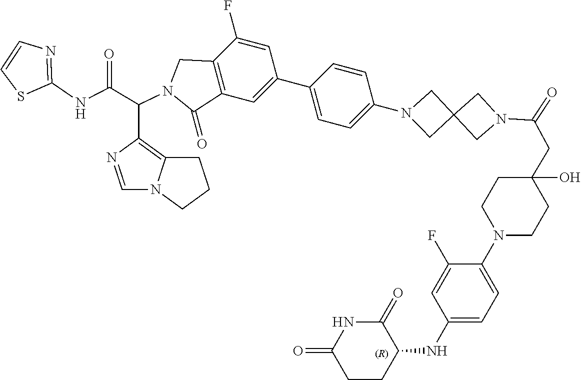 | *** | *** | *** |
| | 2-(6,7-dihydro-5H-pyrrolo[1,2-c]imidazol-1-yl)-2-(6-(4-(6- | | | |
| | (2-(1-(4-(((R)-2,6-dioxopiperidin-3-yl)amino)-2- | | | |
| | fluorophenyl)-4-hydroxypiperidin-4-yl)acetyl)-2,6- | | | |
| | diazaspiro[3.3]heptan-2-yl)phenyl)-4-fluoro-1-oxoisoindolin- | | | |
| | 2-yl)-N-(thiazol-2-yl)acetamide | | | |
| 99 | 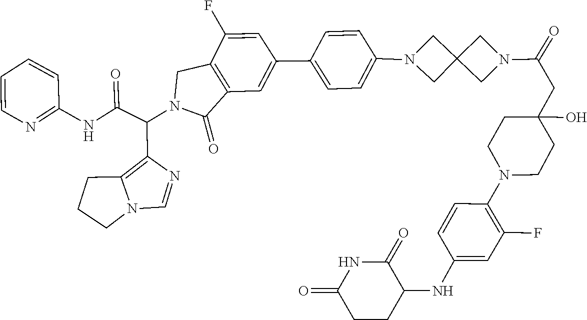 [SMILES: O=C1CCC(Nc2ccc(N3CCC(O)(CC(=O)N4CC5(C4)CN(c4ccc(-c6cc(F)c7c(c6)C(=O)N(C(C(=O)Nc6ccccn6)c6ncn8c6CCC8)C7)cc4)C5)CC3)c(F)c2)C(=O)N1] | *** | | *** |
| | 2-(6,7-dihydro-5H-pyrrolo[1,2-c]imidazol-1-yl)-2-(6-(4-(6- | | | |
| | (2-(1-(4-((2,6-dioxopiperidin-3-yl)amino)-2-fluorophenyl)-4- | | | |
| | hydroxypiperidin-4-yl)acetyl)-2,6-diazaspiro[3.3]heptan-2- | | | |
| | yl)phenyl)-4-fluoro-1-oxoisoindolin-2-yl)-N-(pyridin-2- | | | |
| | yl)acetamide | | | |
| 100 | 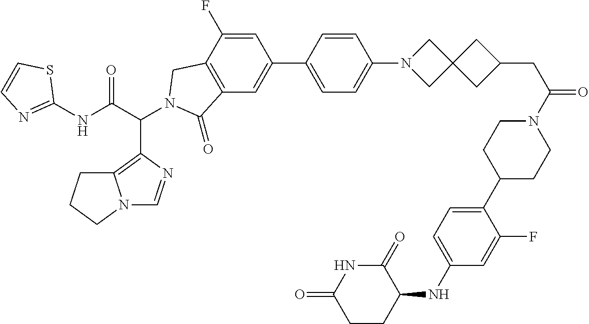 [SMILES: O=C1CC[C@H](Nc2ccc(C3CCN(C(=O)CC4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-(6-(4-(6- | | | |
| | (2-(4-(4-(((S)-2,6-dioxopiperidin-3-yl)amino)-2- | | | |
| | fluorophenyl)piperidin-1-yl)-2-oxoethyl)-2- | | | |
| | azaspiro[3.3]heptan-2-yl)phenyl)-4-fluoro-1-oxoisoindolin-2- | | | |
| | yl)-N-(thiazol-2-yl)acetamide | | | |
| 101 | 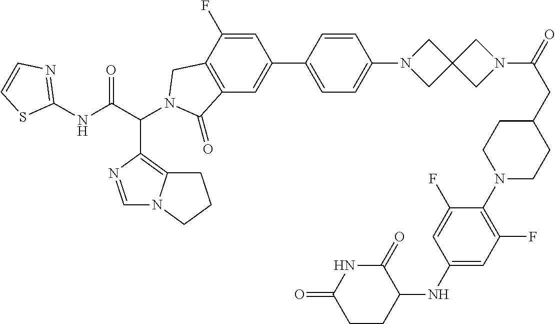 [SMILES: O=C1CCC(Nc2cc(F)c(N3CCC(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | ** | | |
| | 2-(6,7-dihydro-5H-pyrrolo[1,2-c]imidazol-1-yl)-2-(6-(4-(6- | | | |
| | (2-(1-(4-((2,6-dioxopiperidin-3-yl)amino)-2,6- | | | |
| | difluorophenyl)piperidin-4-yl)acetyl)-2,6- | | | |
| | diazaspiro[3.3]heptan-2-yl)phenyl)-4-fluoro-1-oxoisoindolin- | | | |
| | 2-yl)-N-(thiazol-2-yl)acetamide | | | |
| 102 | 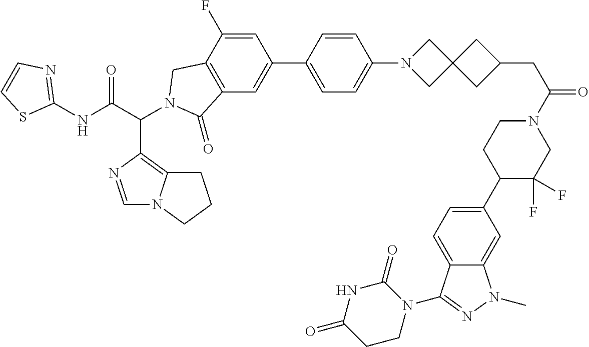 [SMILES: Cn1nc(N2CCC(=O)NC2=O)c2ccc(C3CCN(C(=O)CC4CC5(C4)CN(c4ccc(-c6cc(F)c7c(c6)C(=O)N(C(C(=O)Nc6nccs6)c6ncn8c6CCC8)C7)cc4)C5)CC3(F)F)cc21] | *** | | |
| | 2-(6,7-dihydro-5H-pyrrolo[1,2-c]imidazol-1-yl)-2-(6-(4-(6- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)-2-oxoethyl)-2- | | | |
| | azaspiro[3.3]heptan-2-yl)phenyl)-4-fluoro-1-oxoisoindolin-2- | | | |
| | yl)-N-(thiazol-2-yl)acetamide | | | |
| 103 | 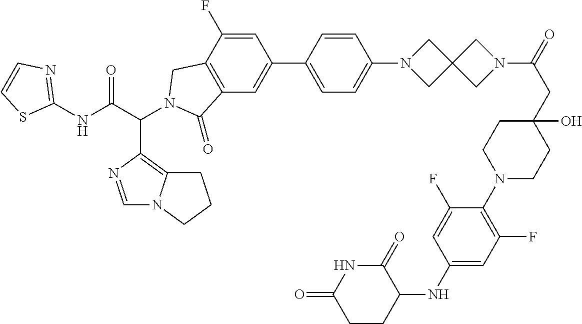 [SMILES: O=C1CCC(Nc2cc(F)c(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | *** | |
| | 2-(6,7-dihydro-5H-pyrrolo[1,2-c]imidazol-1-yl)-2-(6-(4-(6- | | | |
| | (2-(1-(4-((2,6-dioxopiperidin-3-yl)amino)-2,6- | | | |
| | difluorophenyl)-4-hydroxypiperidin-4-yl)acetyl)-2,6- | | | |
| | diazaspiro[3.3]heptan-2-yl)phenyl)-4-fluoro-1-oxoisoindolin- | | | |
| | 2-yl)-N-(thiazol-2-yl)acetamide | | | |
| 104 | 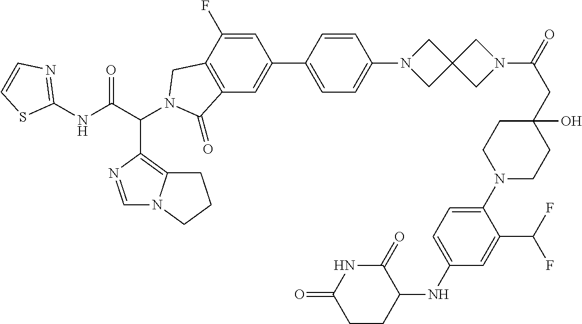 [SMILES: O=C1CCC(Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(C(F)F)c2)C(=O)N1] | *** | *** | |
| | 2-(6-(4-(6-(2-(1-(2-(difluoromethyl)-4-((2,6-dioxopiperidin- | | | |
| | 3-yl)amino)phenyl)-4-hydroxypiperidin-4-yl)acetyl)-2,6- | | | |
| | diazaspiro[3.3]heptan-2-yl)phenyl)-4-fluoro-1-oxoisoindolin- | | | |
| | 2-yl)-2-(6,7-dihydro-5H-pyrrolo[1,2-c]imidazol-1-yl)-N- | | | |
| | (thiazol-2-yl)acetamide | | | |
| 105 | 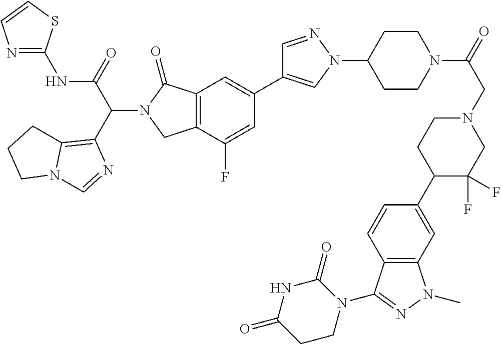 [SMILES: Cn1nc(N2CCC(=O)NC2=O)c2ccc(C3CCN(CC(=O)N4CCC(n5cc(-c6cc(F)c7c(c6)C(=O)N(C(C(=O)Nc6nccs6)c6ncn8c6CCC8)C7)cn5)CC4)CC3(F)F)cc21] | *** | | |
| | 2-(6,7-dihydro-5H-pyrrolo[1,2-c]imidazol-1-yl)-2-(6-(1-(1- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)acetyl)piperidin- | | | |
| | 4-yl)-1H-pyrazol-4-yl)-4-fluoro-1-oxoisoindolin-2-yl)-N- | | | |
| | (thiazol-2-yl)acetamide | | | |
| 106 | 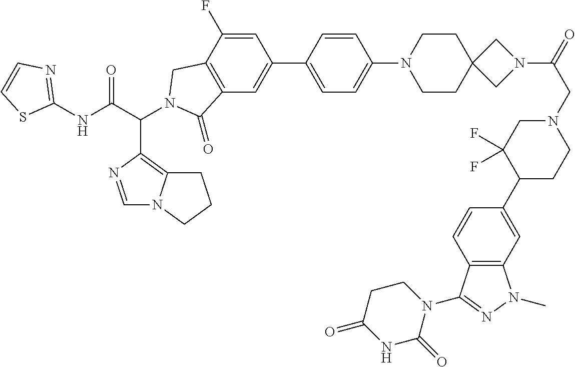 [SMILES: Cn1nc(N2CCC(=O)NC2=O)c2ccc(C3CCN(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3(F)F)cc21] | *** | | |
| | 2-(6,7-dihydro-5H-pyrrolo[1,2-c]imidazol-1-yl)-2-(6-(4-(2- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)acetyl)-2,7- | | | |
| | diazaspiro[3.5]nonan-7-yl)phenyl)-4-fluoro-1-oxoisoindolin- | | | |
| | 2-yl)-N-(thiazol-2-yl)acetamide | | | |
| 107 | 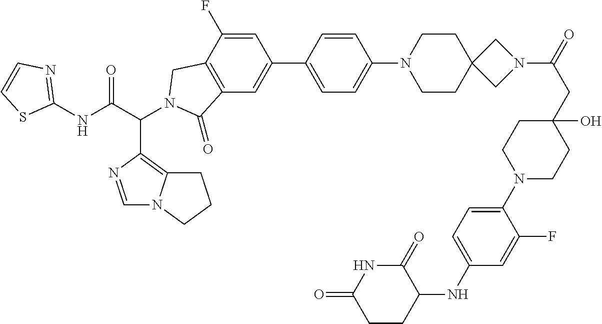 [SMILES: O=C1CCC(Nc2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1] | *** | | |
| | 2-(6,7-dihydro-5H-pyrrolo[1,2-c]imidazol-1-yl)-2-(6-(4-(2- | | | |
| | (2-(1-(4-((2,6-dioxopiperidin-3-yl)amino)-2-fluorophenyl)-4- | | | |
| | hydroxypiperidin-4-yl)acetyl)-2,7-diazaspiro[3.5]nonan-7- | | | |
| | yl)phenyl)-4-fluoro-1-oxoisoindolin-2-yl)-N-(thiazol-2- | | | |
| | yl)acetamide | | | |
| 108 | 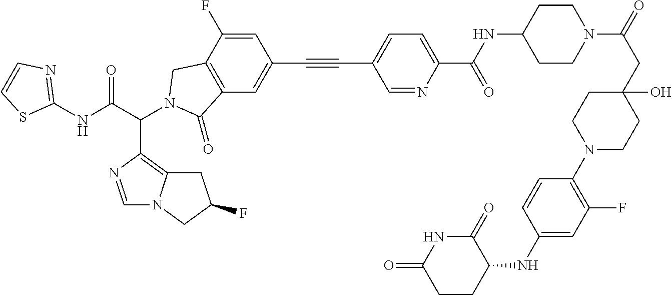 [SMILES: O=C1CC[C@@H](Nc2ccc(N3CCC(O)(CC(=O)N4CCC(NC(=O)c5ccc(C#Cc6cc(F)c7c(c6)C(=O)N(C(C(=O)Nc6nccs6)c6ncn8c6C[C@@H](F)C8)C7)cn5)CC4)CC3)c(F)c2)C(=O)N1] | *** | | |
| | N-(1-(2-(1-(4-(((R)-2,6-dioxopiperidin-3-yl)amino)-2- | | | |
| | fluorophenyl)-4-hydroxypiperidin-4-yl)acetyl)piperidin-4- | | | |
| | yl)-5-((7-fluoro-2-(1-((R)-6-fluoro-6,7-dihydro-5H- | | | |
| | pyrrolo[1,2-c]imidazol-1-yl)-2-oxo-2-(thiazol-2- | | | |
| | ylamino)ethyl)-3-oxoisoindolin-5-yl)ethynyl)picolinamide | | | |
| 109 | 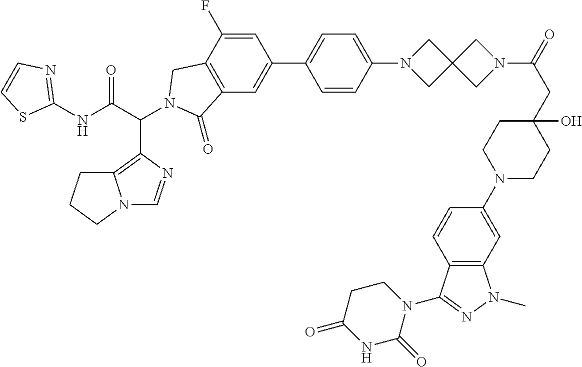 [SMILES: Cn1nc(N2CCC(=O)NC2=O)c2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)cc21] | *** | | |
| | 2-(6,7-dihydro-5H-pyrrolo[1,2-c]imidazol-1-yl)-2-(6-(4-(6- | | | |
| | (2-(1-(3-(2,4-dioxotetrahydropyrimidin-1(2H)-yl)-1-methyl- | | | |
| | 1H-indazol-6-yl)-4-hydroxypiperidin-4-yl)acetyl)-2,6- | | | |
| | diazaspiro[3.3]heptan-2-yl)phenyl)-4-fluoro-1-oxoisoindolin- | | | |
| | 2-yl)-N-(thiazol-2-yl)acetamide | | | |
| 110 | 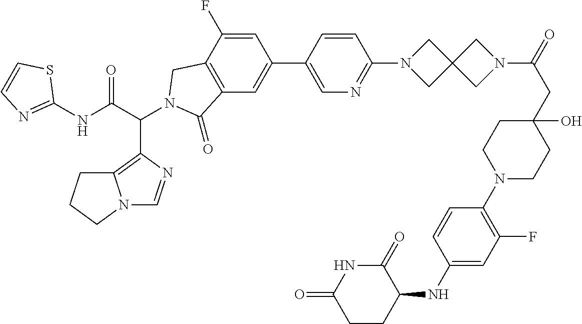 [SMILES: O=C1CC[C@H](Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cn4)C5)CC3)c(F)c2)C(=O)N1] | *** | | |
| | 2-(6,7-dihydro-5H-pyrrolo[1,2-c]imidazol-1-yl)-2-(6-(6-(6- | | | |
| | (2-(1-(4-(((S)-2,6-dioxopiperidin-3-yl)amino)-2- | | | |
| | fluorophenyl)-4-hydroxypiperidin-4-yl)acetyl)-2,6- | | | |
| | diazaspiro[3.3]heptan-2-yl)pyridin-3-yl)-4-fluoro-1- | | | |
| | oxoisoindolin-2-yl)-N-(thiazol-2-yl)acetamide | | | |
| 111 | 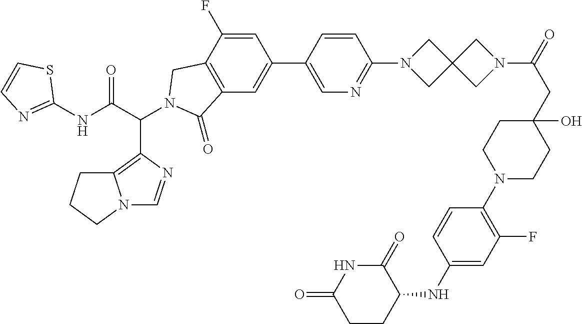 [SMILES: O=C1CC[C@@H](Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cn4)C5)CC3)c(F)c2)C(=O)N1] | *** | | |
| | 2-(6,7-dihydro-5H-pyrrolo[1,2-c]imidazol-1-yl)-2-(6-(6-(6- | | | |
| | (2-(1-(4-(((R)-2,6-dioxopiperidin-3-yl)amino)-2- | | | |
| | fluorophenyl)-4-hydroxypiperidin-4-yl)acetyl)-2,6- | | | |
| | diazaspiro[3.3]heptan-2-yl)pyridin-3-yl)-4-fluoro-1- | | | |
| | oxoisoindolin-2-yl)-N-(thiazol-2-yl)acetamide | | | |
| 112 | 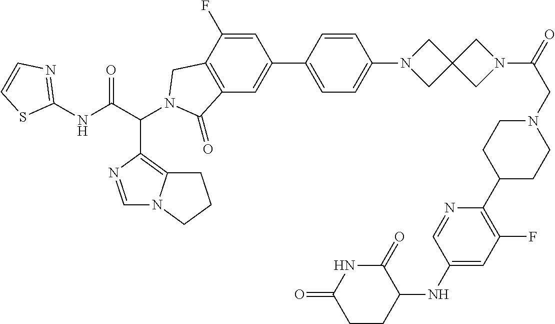 [SMILES: O=C1CCC(Nc2cnc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-(6-(4-(6- | | | |
| | (2-(4-(5-((2,6-dioxopiperidin-3-yl)amino)-3-fluoropyridin-2- | | | |
| | yl)piperidin-1-yl)acetyl)-2,6-diazaspiro[3.3]heptan-2- | | | |
| | yl)phenyl)-4-fluoro-1-oxoisoindolin-2-yl)-N-(thiazol-2- | | | |
| | yl)acetamide | | | |
| 113 | 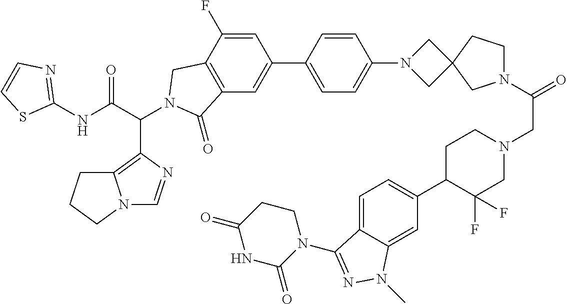 [SMILES: Cn1nc(N2CCC(=O)NC2=O)c2ccc(C3CCN(CC(=O)N4CCC5(C4)CN(c4ccc(-c6cc(F)c7c(c6)C(=O)N(C(C(=O)Nc6nccs6)c6ncn8c6CCC8)C7)cc4)C5)CC3(F)F)cc21] | *** | | |
| | 2-(6,7-dihydro-5H-pyrrolo[1,2-c]imidazol-1-yl)-2-(6-(4-(6- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)acetyl)-2,6- | | | |
| | diazaspiro[3.4]octan-2-yl)phenyl)-4-fluoro-1-oxoisoindolin- | | | |
| | 2-yl)-N-(thiazol-2-yl)acetamide | | | |
| 114 | 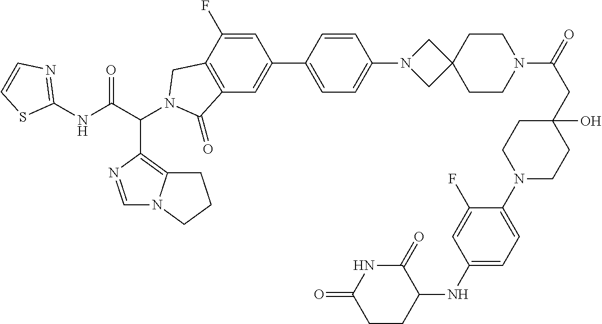 [SMILES: O=C1CCC(Nc2ccc(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-[6-[4-[7- | | | |
| | [2-[1-[4-[(2,6-dioxo-3-piperidyl)amino]-2-fluoro-phenyl]-4- | | | |
| | hydroxy-4-piperidyl]acetyl]-2,7-diazaspiro[3.5]nonan-2- | | | |
| | yl]phenyl]-4-fluoro-1-oxo-isoindolin-2-yl]-N-thiazol-2-yl- | | | |
| | acetamide | | | |
| 115 | 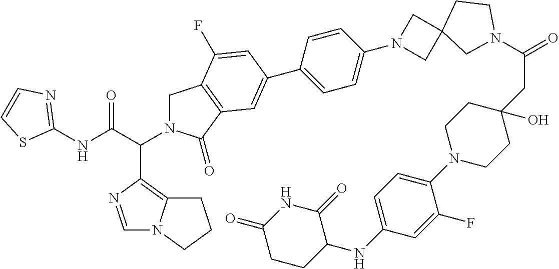 [SMILES: O=C1CCC(Nc2ccc(N3CCC(O)(CC(=O)N4CCC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-[6-[4-[7- | | | |
| | [2-[1-[4-[(2,6-dioxo-3-piperidyl)amino]-2-fluoro-phenyl]-4- | | | |
| | hydroxy-4-piperidyl]acetyl]-2,7-diazaspiro[3.4]octan-2- | | | |
| | yl]phenyl]-4-fluoro-1-oxo-isoindolin-2-yl]-N-thiazol-2-yl- | | | |
| | acetamide | | | |
| 116 | 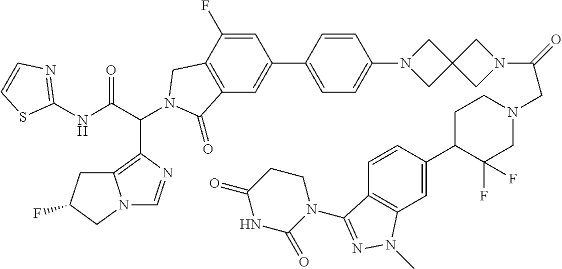 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6C[C@@H](F)C8)C7)cc4)C5)CC3(F)F)cc21] | *** | | |
| | 2-[6-[4-[2-[2-[4-[3-(2,4-dioxohexahydropyrimidin-1-yl)-1- | | | |
| | methyl-indazol-6-yl]-3,3-difluoro-1-piperidyl]acetyl]-2,6- | | | |
| | diazaspiro[3.3]heptan-6-yl]phenyl]-4-fluoro-1-oxo- | | | |
| | isoindolin-2-yl]-2-[(6R)-6-fluoro-6,7-dihydro-5H- | | | |
| | pyrrolo[1,2-c]imidazol-1-yl]-N-thiazol-2-yl-acetamide | | | |
| 117 | 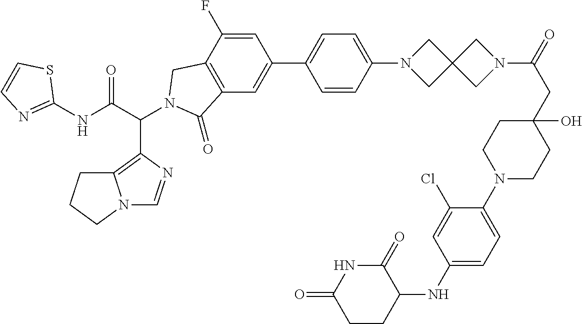 [SMILES: O=C1CCC(Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1] | *** | | |
| | 2-[6-[4-[2-[2-[1-[2-chloro-4-[(2,6-dioxo-3- | | | |
| | piperidyl)amino]phenyl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-2-(6,7-dihydro-5H-pyrrolo[1,2-c]imidazol-1- | | | |
| | yl)-N-thiazol-2-yl-acetamide | | | |
| 118 | 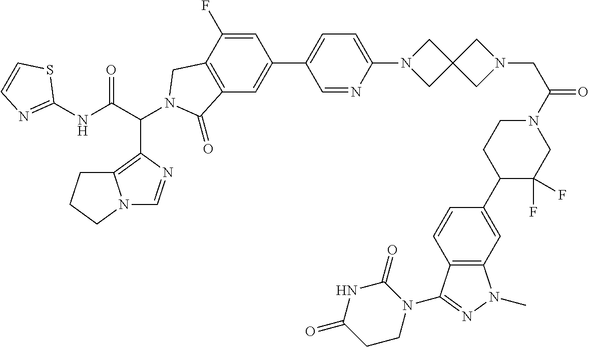 [SMILES: Cn1nc(N2CCC(=O)NC2=O)c2ccc(C3CCN(C(=O)CN4CC5(C4)CN(c4ccc(-c6cc(F)c7c(c6)C(=O)N(C(C(=O)Nc6nccs6)c6ncn8c6CCC8)C7)cn4)C5)CC3(F)F)cc21] | *** | | |
| | 2-(6,7-dihydro-5H-pyrrolo[1,2-c]imidazol-1-yl)-2-(6-(6-(6- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)-2-oxoethyl)- | | | |
| | 2,6-diazaspiro[3.3]heptan-2-yl)pyridin-3-yl)-4-fluoro-1- | | | |
| | oxoisoindolin-2-yl)-N-(thiazol-2-yl)acetamide | | | |
| 119 | 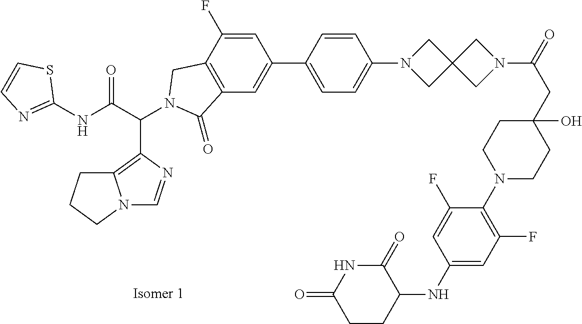 [SMILES: O=C1CCC(Nc2cc(F)c(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-(6-(4-(6- | | | |
| | (2-(1-(4-((2,6-dioxopiperidin-3-yl)amino)-2,6- | | | |
| | difluorophenyl)-4-hydroxypiperidin-4-yl)acetyl)-2,6- | | | |
| | diazaspiro[3.3]heptan-2-yl)phenyl)-4-fluoro-1-oxoisoindolin- | | | |
| | 2-yl)-N-(thiazol-2-yl)acetamide, isomer 1 | | | |
| 123 | 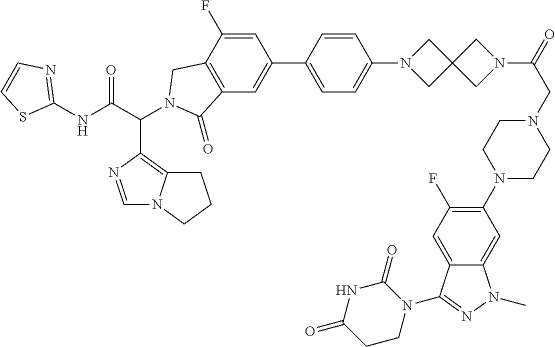 [SMILES: Cn1nc(N2CCC(=O)NC2=O)c2cc(F)c(N3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)cc21] | *** | | |
| | 2-(6,7-dihydro-5H-pyrrolo[1,2-c]imidazol-1-yl)-2-[6-[4-[2- | | | |
| | [2-[4-[3-(2,4-dioxohexahydropyrimidin-1-yl)-5-fluoro-1- | | | |
| | methyl-indazol-6-yl]piperazin-1-yl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide | | | |
| 125 | 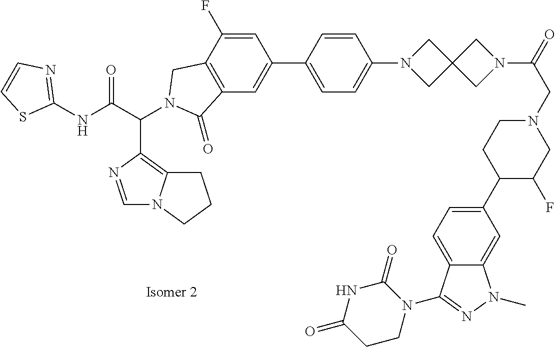 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3F)cc21] | *** | | |
| | 2-(6,7-dihydro-5H-pyrrolo[1,2-c]imidazol-1-yl)-2-[6-[4-[2- | | | |
| | [2-[4-[3-(2,4-dioxohexahydropyrimidin-1-yl)-1-methyl- | | | |
| | indazol-6-yl]-3-fluoro-1-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide, isomer 2 | | | |
| 127 | 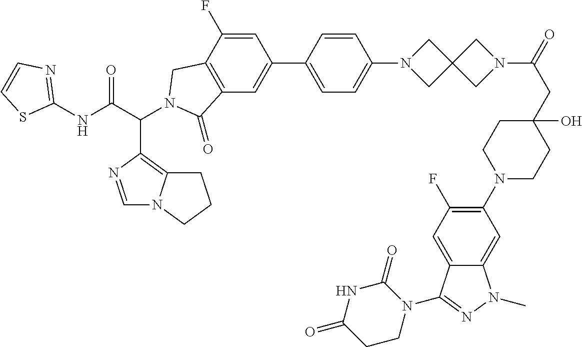 [SMILES: Cn1nc(N2CCC(=O)NC2=O)c2cc(F)c(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)cc21] | *** | | |
| | 2-(6,7-dihydro-5H-pyrrolo[1,2-c]imidazol-1-yl)-2-[6-[4-[2- | | | |
| | [2-[1-[3-(2,4-dioxohexahydropyrimidin-1-yl)-5-fluoro-1- | | | |
| | methyl-indazol-6-yl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide | | | |
| 128 | 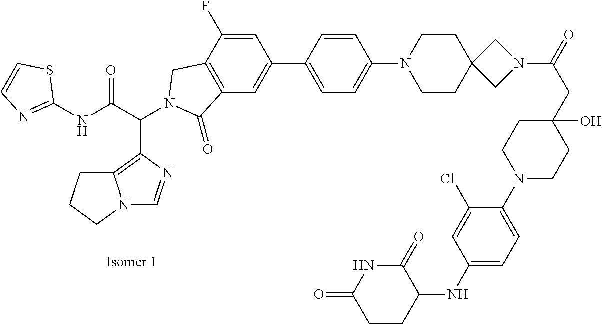 [SMILES: O=C1CCC(Nc2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(Cl)c2)C(=O)N1] | *** | | |
| | 2-[6-[4-[2-[2-[1-[2-chloro-4-[[2,6-dioxo-3- | | | |
| | piperidyl]amino]phenyl]-4-hydroxy-4-piperidyl]acetyl]-2,7- | | | |
| | diazaspiro[3.5]nonan-7-yl]phenyl]-4-fluoro-1-oxo- | | | |
| | isoindolin-2-yl]-2-(6,7-dihydro-5H-pyrrolo[1,2-c]imidazol-1- | | | |
| | yl)-N-thiazol-2-yl-acetamide, isomer 1 | | | |
| 129 | 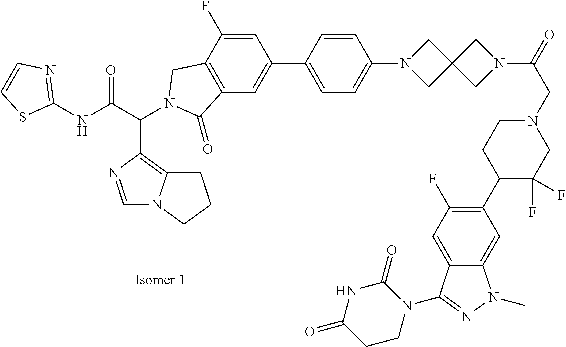 [SMILES: Cn1nc(N2CCC(=O)NC2=O)c2cc(F)c(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21] | *** | | |
| | 2-(6,7-dihydro-5H-pyrrolo[1,2-c]imidazol-1-yl)-2-[6-[4-[2- | | | |
| | [2-[4-[3-(2,4-dioxohexahydropyrimidin-1-yl)-5-fluoro-1- | | | |
| | methyl-indazol-6-yl]-3,3-difluoro-1-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide, isomer 1 | | | |
| 130 | 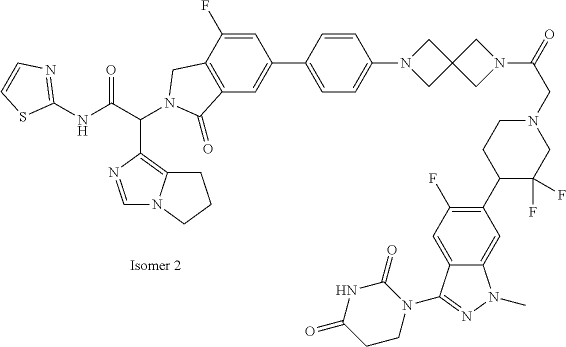 [SMILES: Cn1nc(N2CCC(=O)NC2=O)c2cc(F)c(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3(F)F)cc21] | *** | | |
| | 2-(6,7-dihydro-5H-pyrrolo[1,2-c]imidazol-1-yl)-2-[6-[4-[2- | | | |
| | [2-[4-[3-(2,4-dioxohexahydropyrimidin-1-yl)-5-fluoro-1- | | | |
| | methyl-indazol-6-yl]-3,3-difluoro-1-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide, isomer 2 | | | |
| 131 | 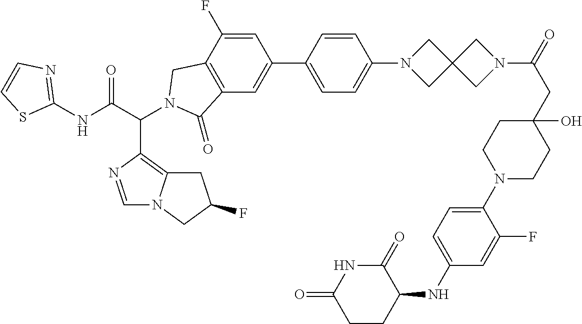 [SMILES: O=C1CC[C@H](Nc2ccc(N3CCC(O)(CC(=O)N4CC5(C4)CN(c4ccc(-c6cc(F)c7c(c6)C(=O)N(C(C(=O)Nc6nccs6)c6ncn8c6C[C@@H](F)C8)C7)cc4)C5)CC3)c(F)c2)C(=O)N1] | *** | | |
| | 2-[6-[4-[2-[2-[1-[4-[[(3S)-2,6-dioxo-3-piperidyl]amino]-2- | | | |
| | fluoro-phenyl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-2-[(6R)-6-fluoro-6,7-dihydro-5H- | | | |
| | pyrrolo[1,2-c]imidazol-1-yl]-N-thiazol-2-yl-acetamide | | | |
| 132 | 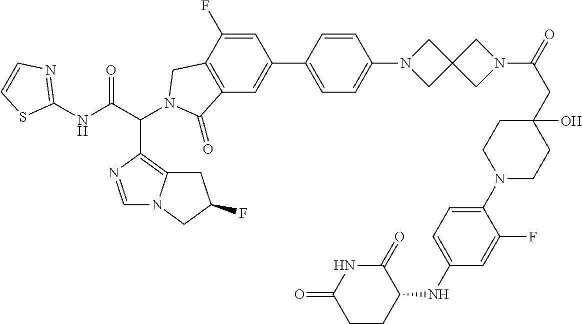 [SMILES: O=C1CC[C@@H](Nc2ccc(N3CCC(O)(CC(=O)N4CC5(C4)CN(c4ccc(-c6cc(F)c7c(c6)C(=O)N(C(C(=O)Nc6nccs6)c6ncn8c6C[C@@H](F)C8)C7)cc4)C5)CC3)c(F)c2)C(=O)N1] | *** | | |
| | 2-[6-[4-[2-[2-[1-[4-[[(3R)-2,6-dioxo-3-piperidyl]amino]-2- | | | |
| | fluoro-phenyl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-2-[(6R)-6-fluoro-6,7-dihydro-5H- | | | |
| | pyrrolo[1,2-c]imidazol-1-yl]-N-thiazol-2-yl-acetamide | | | |
| 133 | 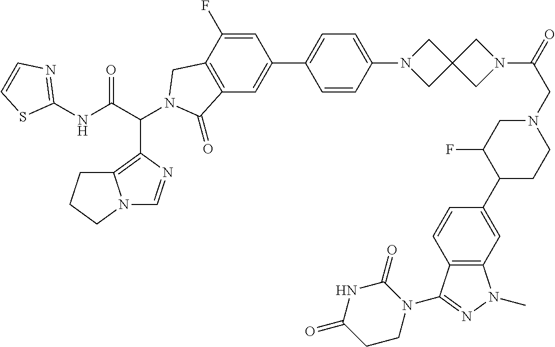 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)CC3F)cc21] | *** | | |
| | 2-(6,7-dihydro-5H-pyrrolo[1,2-c]imidazol-1-yl)-2-[6-[4-[2- | | | |
| | [2-[4-[3-(2,4-dioxohexahydropyrimidin-1-yl)-1-methyl- | | | |
| | indazol-6-yl]-3-fluoro-1-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide | | | |
| 134 | 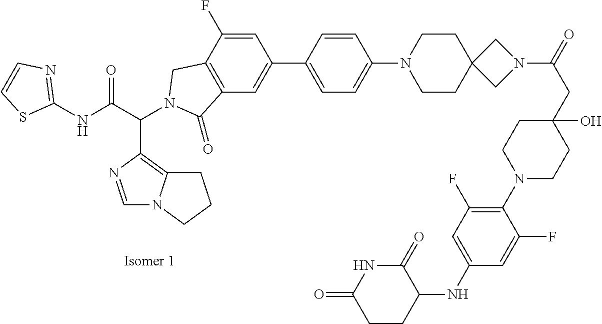 [SMILES: O=C1CCC(Nc2cc(F)c(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1] | *** | | |
| | 2-(6,7-dihydro-5H-pyrrolo[1,2-c]imidazol-1-yl)-2-[6-[4-[2- | | | |
| | [2-[1-[4-[[2,6-dioxo-3-piperidyl]amino]-2,6-difluoro- | | | |
| | phenyl]-4-hydroxy-4-piperidyl]acetyl]-2,7- | | | |
| | diazaspiro[3.5]nonan-7-yl]phenyl]-4-fluoro-1-oxo- | | | |
| | isoindolin-2-yl]-N-thiazol-2-yl-acetamide, isomer 1 | | | |
| 135 | 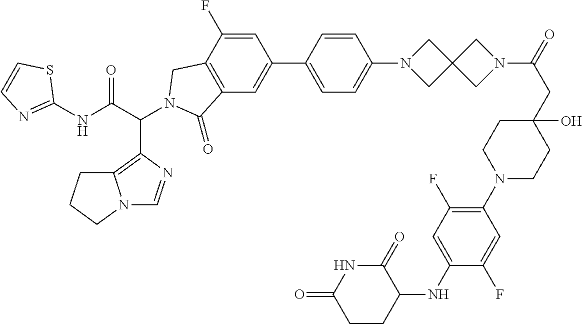 [SMILES: O=C1CCC(Nc2cc(F)c(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)cc2F)C(=O)N1] | *** | | |
| | 2-(6,7-dihydro-5H-pyrrolo[1,2-c]imidazol-1-yl)-2-[6-[4-[2- | | | |
| | [2-[1-[4-[(2,6-dioxo-3-piperidyl)amino]-2,5-difluoro- | | | |
| | phenyl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide | | | |
| 136 | 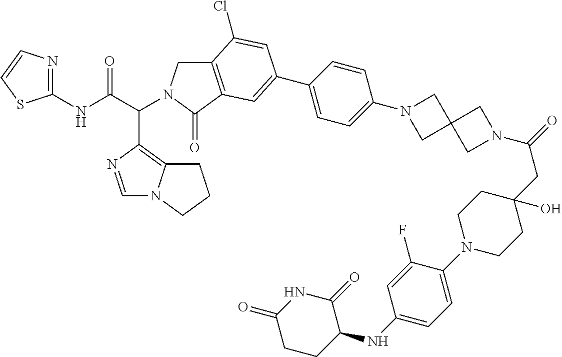 [SMILES: O=C1CC[C@H](Nc2ccc(N3CCC(O)(CC(=O)N4CC5(C4)CN(c4ccc(-c6cc(Cl)c7c(c6)C(=O)N(C(C(=O)Nc6nccs6)c6ncn8c6CCC8)C7)cc4)C5)CC3)c(F)c2)C(=O)N1] | *** | | |
| | 2-[4-chloro-6-[4-[2-[2-[1-[4-[[(3S)-2,6-dioxo-3- | | | |
| | piperidyl]amino]-2-fluoro-phenyl]-4-hydroxy-4- | | | |
| | piperidyl]acetyl]-2,6-diazaspiro[3.3]heptan-6-yl]phenyl]-1- | | | |
| | oxo-isoindolin-2-yl]-2-(6,7-dihydro-5H-pyrrolo[1,2- | | | |
| | c]imidazol-1-yl)-N-thiazol-2-yl-acetamide | | | |
| 137 | 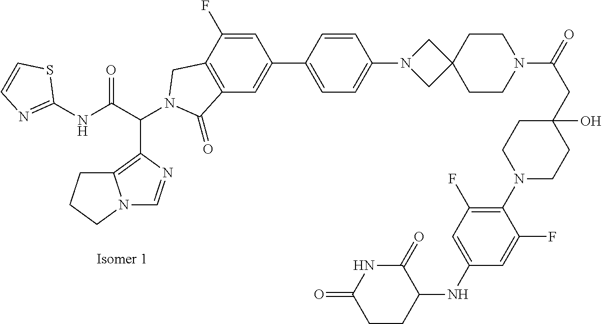 [SMILES: O=C1CCC(Nc2cc(F)c(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | ** | | |
| | 2-(6,7-dihydro-5H-pyrrolo[1,2-c]imidazol-1-yl)-2-[6-[4-[7- | | | |
| | [2-[1-[4-[2,6-dioxo-3-piperidyl]amino]-2,6-difluoro- | | | |
| | phenyl]-4-hydroxy-4-piperidyl]acetyl]-2,7- | | | |
| | diazaspiro[3.5]nonan-2-yl]phenyl]-4-fluoro-1-oxo- | | | |
| | isoindolin-2-yl]-N-thiazol-2-yl-acetamide, isomer 1 | | | |
| 138 | 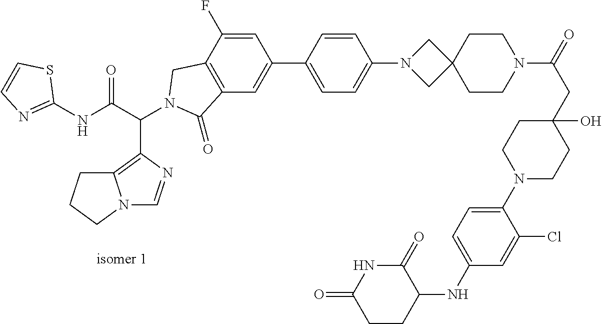 [SMILES: O=C1CCC(Nc2ccc(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1] | *** | | |
| | 2-[6-[4-[7-[2-[1-[2-chloro-4-[[2,6-dioxo-3- | | | |
| | piperidyl]amino]phenyl]-4-hydroxy-4-piperidyl]acetyl]-2,7- | | | |
| | diazaspiro[3.5]nonan-2-yl]phenyl]-4-fluoro-1-oxo- | | | |
| | isoindolin-2-yl]-2-(6,7-dihydro-5H-pyrrolo[1,2-c]imidazol-1- | | | |
| | yl)-N-thiazol-2-yl-acetamide, isomer 1 | | | |
| 139 | 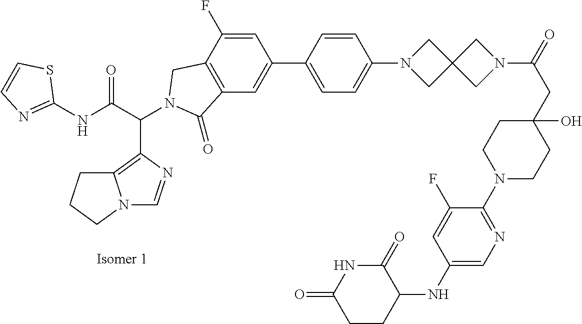 [SMILES: O=C1CCC(Nc2cnc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-[6-[4-[2- | | | |
| | [2-[1-[5-[[2,6-dioxo-3-piperidyl]amino]-3-fluoro-2-pyridyl]- | | | |
| | 4-hydroxy-4-piperidyl]acetyl]-2,6-diazaspiro[3.3]heptan-6- | | | |
| | yl]phenyl]-4-fluoro-1-oxo-isoindolin-2-yl]-N-thiazol-2-yl- | | | |
| | acetamide, isomer 1 | | | |
| 140 | 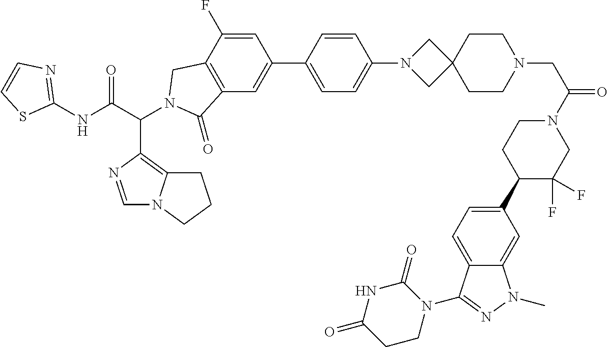 [SMILES: Cn1nc(N2CCC(=O)NC2=O)c2ccc([C@H]3CCN(C(=O)CN4CCC5(CC4)CN(c4ccc(-c6cc(F)c7c(c6)C(=O)N(C(C(=O)Nc6nccs6)c6ncn8c6CCC8)C7)cc4)C5)CC3(F)F)cc21] | *** | | |
| | 2-(6,7-dihydro-5H-pyrrolo[1,2-c]imidazol-1-yl)-2-[6-[4-[7- | | | |
| | [2-[(4R)-4-[3-(2,4-dioxohexahydropyrimidin-1-yl)-1-methyl- | | | |
| | indazol-6-yl]-3,3-difluoro-1-piperidyl]-2-oxo-ethyl]-2,7- | | | |
| | diazaspiro[3.5]nonan-2-yl]phenyl]-4-fluoro-1-oxo- | | | |
| | isoindolin-2-yl]-N-thiazol-2-yl-acetamide | | | |
| 141 | 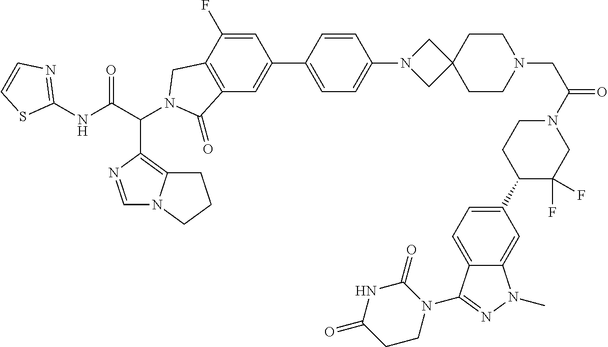 [SMILES: Cn1nc(N2CCC(=O)NC2=O)c2ccc([C@@H]3CCN(C(=O)CN4CCC5(CC4)CN(c4ccc(-c6cc(F)c7c(c6)C(=O)N(C(C(=O)Nc6nccs6)c6ncn8c6CCC8)C7)cc4)C5)CC3(F)F)cc21] | ** | | |
| | 2-(6,7-dihydro-5H-pyrrolo[1,2-c]imidazol-1-yl)-2-[6-[4-[7- | | | |
| | [2-[(4S)-4-[3-(2,4-dioxohexahydropyrimidin-1-yl)-1-methyl- | | | |
| | indazol-6-yl]-3,3-difluoro-1-piperidyl]-2-oxo-ethyl]-2,7- | | | |
| | diazaspiro[3.5]nonan-2-yl]phenyl]-4-fluoro-1-oxo- | | | |
| | isoindolin-2-yl]-N-thiazol-2-yl-acetamide | | | |
| 142 | 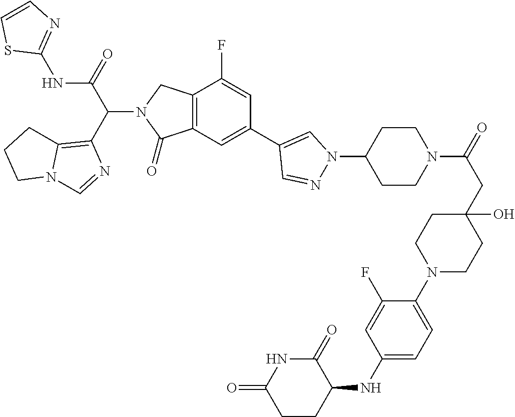 [SMILES: O=C1CC[C@H](Nc2ccc(N3CCC(O)(CC(=O)N4CCC(n5cc(-c6cc(F)c7c(c6)C(=O)N(C(C(=O)Nc6nccs6)c6ncn8c6CCC8)C7)cn5)CC4)CC3)c(F)c2)C(=O)N1] | *** | | |
| | 2-(6,7-dihydro-5H-pyrrolo[1,2-c]imidazol-1-yl)-2-[6-[1-[1- | | | |
| | [2-[1-[4-[[(3S)-2,6-dioxo-3-piperidyl]amino]-2-fluoro- | | | |
| | phenyl]-4-hydroxy-4-piperidyl]acetyl]-4-piperidyl]pyrazol-4- | | | |
| | yl]-4-fluoro-1-oxo-isoindolin-2-yl]-N-thiazol-2-yl-acetamide | | | |
| 143 | 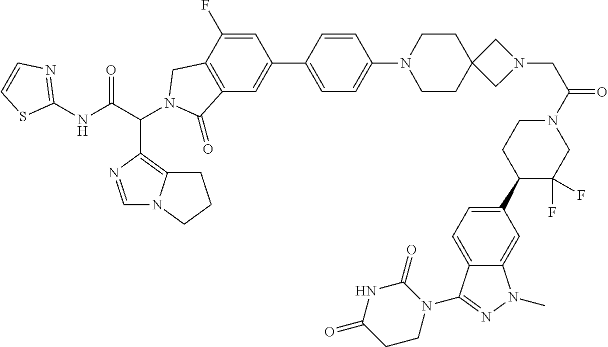 [SMILES: Cn1nc(N2CCC(=O)NC2=O)c2ccc([C@H]3CCN(C(=O)CN4CC5(CCN(c6ccc(-c7cc(F)c8c(c7)C(=O)N(C(C(=O)Nc7nccs7)c7ncn9c7CCC9)C8)cc6)CC5)C4)CC3(F)F)cc21] | ** | | |
| | 2-(6,7-dihydro-5H-pyrrolo[1,2-c]imidazol-1-yl)-2-[6-[4-[2- | | | |
| | [2-[(4R)-4-[3-(2,4-dioxohexahydropyrimidin-1-yl)-1-methyl- | | | |
| | indazol-6-yl]-3,3-difluoro-1-piperidyl]-2-oxo-ethyl]-2,7- | | | |
| | diazaspiro[3.5]nonan-7-yl]phenyl]-4-fluoro-1-oxo- | | | |
| | isoindolin-2-yl]-N-thiazol-2-yl-acetamide | | | |
| 144 | 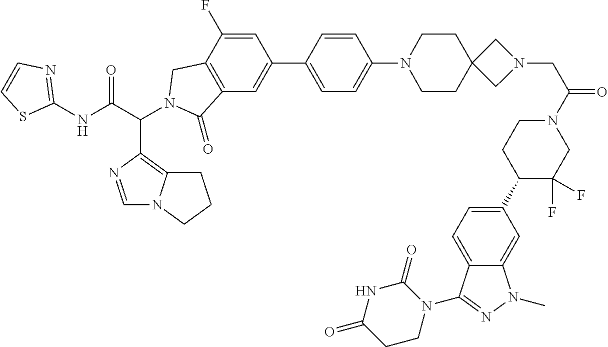 [SMILES: Cn1nc(N2CCC(=O)NC2=O)c2ccc([C@@H]3CCN(C(=O)CN4CC5(CCN(c6ccc(-c7cc(F)c8c(c7)C(=O)N(C(C(=O)Nc7nccs7)c7ncn9c7CCC9)C8)cc6)CC5)C4)CC3(F)F)cc21] | *** | | |
| | 2-(6,7-dihydro-5H-pyrrolo[1,2-c]imidazol-1-yl)-2-[6-[4-[2- | | | |
| | [2-[(4S)-4-[3-(2,4-dioxohexahydropyrimidin-1-yl)-1-methyl- | | | |
| | indazol-6-yl]-3,3-difluoro-1-piperidyl]-2-oxo-ethyl]-2,7- | | | |
| | diazaspiro[3.5]nonan-7-yl]phenyl]-4-fluoro-1-oxo- | | | |
| | isoindolin-2-yl]-N-thiazol-2-yl-acetamide | | | |
| 145 | 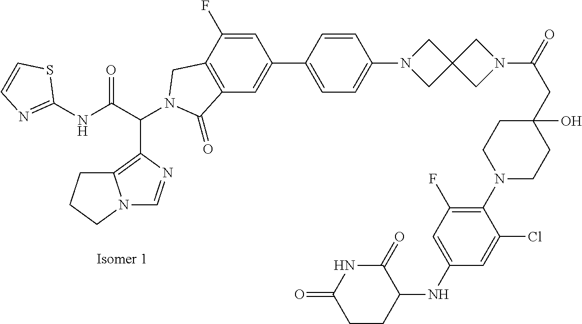 [SMILES: O=C1CCC(Nc2cc(F)c(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1] | ** | | |
| | 2-[6-[4-[2-[2-[1-[2-chloro-4-[[2,6-dioxo-3-piperidyl]amino]- | | | |
| | 6-fluoro-phenyl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-2-(6,7-dihydro-5H-pyrrolo[1,2-c]imidazol-1- | | | |
| | yl)-N-thiazol-2-yl-acetamide, isomer 1 | | | |
| 146 | 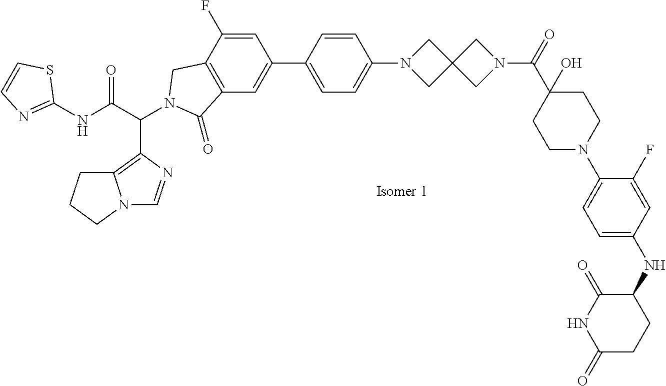 [SMILES: O=C1CC[C@H](Nc2ccc(N3CCC(O)(C(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-[6-[4-[2- | | | |
| | [1-[4-[[2,6-dioxo-3-piperidyl]amino]-2-fluoro-phenyl]-4- | | | |
| | hydroxy-piperidine-4-carbonyl]-2,6-diazaspiro[3.3]heptan-6- | | | |
| | yl]phenyl]-4-fluoro-1-oxo-isoindolin-2-yl]-N-thiazol-2-yl- | | | |
| | acetamide, isomer 1 | | | |
| 147 | 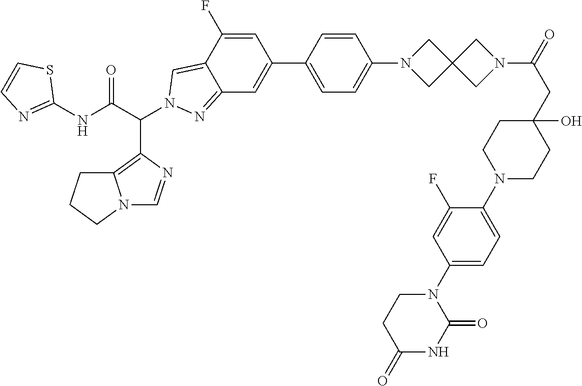 [SMILES: O=C1CCN(c2ccc(N3CCC(O)(CC(=O)N4CC5(C4)CN(c4ccc(-c6cc(F)c7cn(C(C(=O)Nc8nccs8)c8ncn9c8CCC9)nc7c6)cc4)C5)CC3)c(F)c2)C(=O)N1] | | | *** |
| | 2-(6,7-dihydro-5H-pyrrolo[1,2-c]imidazol-1-yl)-2-[6-[4-[2- | | | |
| | [2-[1-[4-(2,4-dioxohexahydropyrimidin-1-yl)-2-fluoro- | | | |
| | phenyl]-4-hydroxy-4-piperidyllacetyl]-2,6- | | | |
| | diazaspiro[3.3]heptan-6-yl]phenyl]-4-fluoro-indazol-2-yl]-N- | | | |
| | thiazol-2-yl-acetamide | | | |
| 148 | 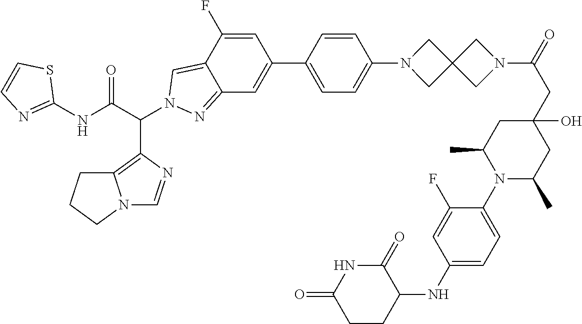 [SMILES: C[C@@H]1CC(O)(CC(=O)N2CC3(C2)CN(c2ccc(-c4cc(F)c5cn(C(C(=O)Nc6nccs6)c6ncn7c6CCC7)nc5c4)cc2)C3)C[C@H](C)N1c1ccc(NC2CCC(=O)NC2=O)cc1F] | | | *** |
| | 2-(6,7-dihydro-5H-pyrrolo[1,2-c]imidazol-1-yl)-2-[6-[4-[2- | | | |
| | [2-[(2s,6r)-1-[4-[(2,6-dioxo-3-piperidyl)amino]-2-fluoro- | | | |
| | phenyl]-4-hydroxy-2,6-dimethyl-4-piperidyl]acetyl]-2,6- | | | |
| | diazaspiro[3.3]heptan-6-yl]phenyl]-4-fluoro-indazol-2-yl]-N- | | | |
| | thiazol-2-yl-acetamide | | | |
| 149 | 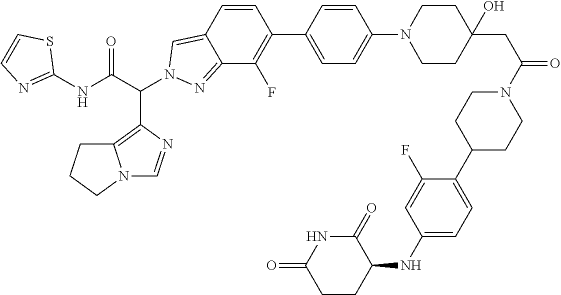 [SMILES: O=C1CC[C@H](Nc2ccc(C3CCN(C(=O)CC4(O)CCN(c5ccc(-c6ccc7cn(C(C(=O)Nc8nccs8)c8ncn9c8CCC9)nc7c6F)cc5)CC4)CC3)c(F)c2)C(=O)N1] | | | *** |
| | 2-(6,7-dihydro-5H-pyrrolo[1,2-c]imidazol-1-yl)-2-[6-[4-[4- | | | |
| | [2-[4-[4-[[(3S)-2,6-dioxo-3-piperidyl]amino]-2-fluoro- | | | |
| | phenyl]-1-piperidyl]-2-oxo-ethyl]-4-hydroxy-1- | | | |
| | piperidyl]phenyl]-7-fluoro-indazol-2-yl]-N-thiazol-2-yl- | | | |
| | acetamide | | | |
| 150 | 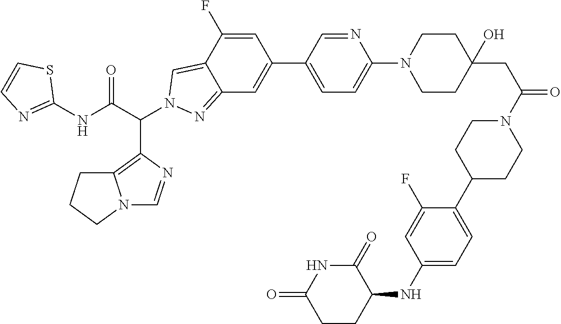 [SMILES: O=C1CC[C@H](Nc2ccc(C3CCN(C(=O)CC4(O)CCN(c5ccc(-c6cc(F)c7cn(C(C(=O)Nc8nccs8)c8ncn9c8CCC9)nc7c6)cn5)CC4)CC3)c(F)c2)C(=O)N1] | | | *** |
| | 2-(6,7-dihydro-5H-pyrrolo[1,2-c]imidazol-1-yl)-2-[6-[6-[4- | | | |
| | [2-[4-[4-[[(3S)-2,6-dioxo-3-piperidyl]amino]-2-fluoro- | | | |
| | phenyl]-1-piperidyl]-2-oxo-ethyl]-4-hydroxy-1-piperidyl]-3- | | | |
| | pyridyl]-4-fluoro-indazol-2-yl]-N-thiazol-2-yl-acetamide | | | |
| 151 | 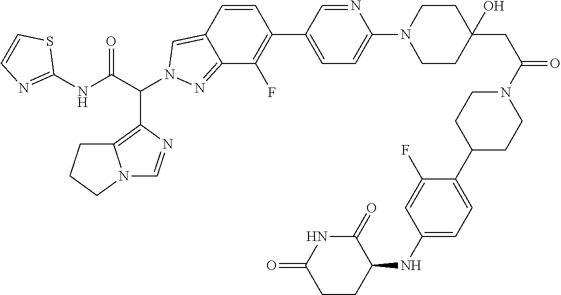 [SMILES: O=C1CC[C@H](Nc2ccc(C3CCN(C(=O)CC4(O)CCN(c5ccc(-c6ccc7cn(C(C(=O)Nc8nccs8)c8ncn9c8CCC9)nc7c6F)cn5)CC4)CC3)c(F)c2)C(=O)N1] | | | *** |
| | 2-(6,7-dihydro-5H-pyrrolo[1,2-c]imidazol-1-yl)-2-[6-[6-[4- | | | |
| | [2-[4-[4-[[(3S)-2,6-dioxo-3-piperidyl]amino]-2-fluoro- | | | |
| | phenyl]-1-piperidyl]-2-oxo-ethyl]-4-hydroxy-1-piperidyl]-3- | | | |
| | pyridyl]-7-fluoro-indazol-2-yl]-N-thiazol-2-yl-acetamide | | | |
| 152 | 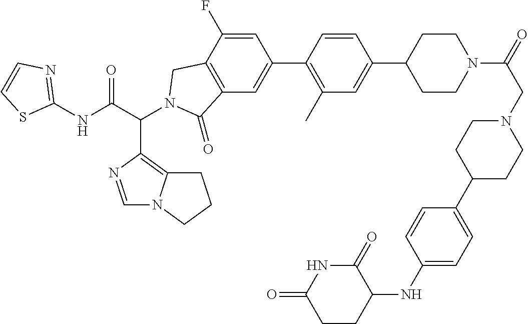 [SMILES: Cc1cc(C2CCN(C(=O)CN3CCC(c4ccc(NC5CCC(=O)NC5=O)cc4)CC3)CC2)ccc1-c1cc(F)c2c(c1)C(=O)N(C(C(=O)Nc1nccs1)c1ncn3c1CCC3)C2] | ** | | |
| | 2-(6,7-dihydro-5H-pyrrolo[1,2-c]imidazol-1-yl)-2-[6-[4-[1- | | | |
| | [2-[4-[4-[(2,6-dioxo-3-piperidyl)amino]phenyl]-1- | | | |
| | piperidyl]acetyl]-4-piperidyl]-2-methyl-phenyl]-4-fluoro-1- | | | |
| | oxo-isoindolin-2-yl]-N-thiazol-2-yl-acetamide | | | |
| 153 | 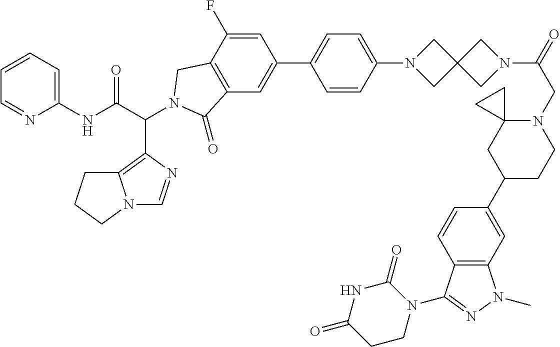 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6ccccn6)c6ncn8c6CCC8)C7)cc4)C5)C4(CC4)C3)cc21] | ** | | |
| | 2-(6,7-dihydro-5H-pyrrolo[1,2-c]imidazol-1-yl)-2-[6-[4-[2- | | | |
| | [2-[7-[3-(2,4-dioxohexahydropyrimidin-1-yl)-1-methyl- | | | |
| | indazol-6-yl]-4-azaspiro[2.5]octan-4-yl]acetyl]-2,6- | | | |
| | diazaspiro[3.3]heptan-6-yl]phenyl]-4-fluoro-1-oxo- | | | |
| | isoindolin-2-yl]-N-(2-pyridyl)acetamide | | | |
| 154 | 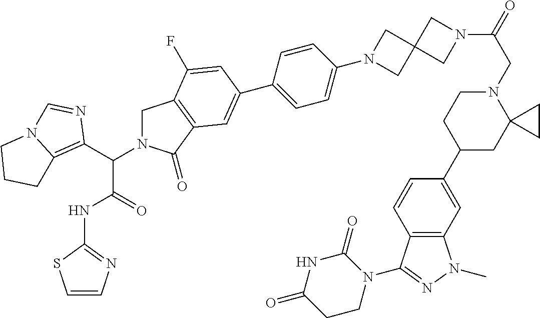 [SMILES: Cn1nc(N2CCC(=O)NC2=O)c2ccc(C3CCN(CC(=O)N4CC5(C4)CN(c4ccc(-c6cc(F)c7c(c6)C(=O)N(C(C(=O)Nc6nccs6)c6ncn8c6CCC8)C7)cc4)C5)C4(CC4)C3)cc21] | *** | | *** |
| | 2-(6,7-dihydro-5H-pyrrolo[1,2-c]imidazol-1-yl)-2-[6-[4-[2- | | | |
| | [2-[7-[3-(2,4-dioxohexahydropyrimidin-1-yl)-1-methyl- | | | |
| | indazol-6-yl]-4-azaspiro[2.5]octan-4-yl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide | | | |
| 155 | 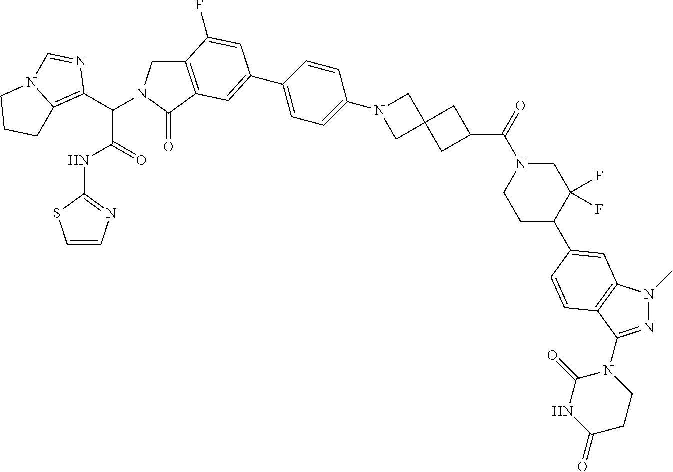 [SMILES: Cn1nc(N2CCC(=O)NC2=O)c2ccc(C3CCN(C(=O)C4CC5(C4)CN(c4ccc(-c6cc(F)c7c(c6)C(=O)N(C(C(=O)Nc6nccs6)c6ncn8c6CCC8)C7)cc4)C5)CC3(F)F)cc21] | *** | | |
| | 2-(6,7-dihydro-5H-pyrrolo[1,2-c]imidazol-1-yl)-2-[6-[4-[6- | | | |
| | [4-[3-(2,4-dioxohexahydropyrimidin-1-yl)-1-methyl-indazol- | | | |
| | 6-yl]-3,3-difluoro-piperidine-1-carbonyl]-2- | | | |
| | azaspiro[3.3]heptan-2-yl]phenyl]-4-fluoro-1-oxo-isoindolin- | | | |
| | 2-yl]-N-thiazol-2-yl-acetamide | | | |
| 156 | 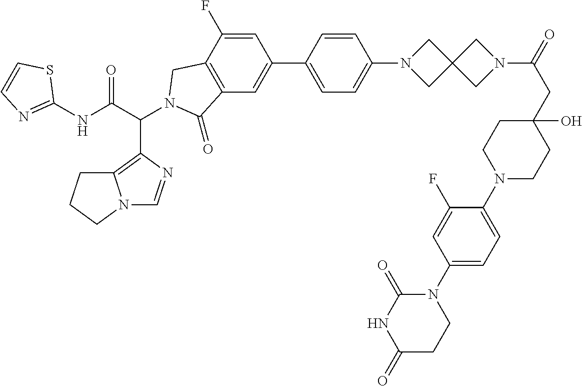 [SMILES: O=C1CCN(c2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | ** | | |
| | 2-(6,7-dihydro-5H-pyrrolo[1,2-c]imidazol-1-yl)-2-[6-[4-[2- | | | |
| | [2-[1-[4-(2,4-dioxohexahydropyrimidin-1-yl)-2-fluoro- | | | |
| | phenyl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide | | | |
| 157 | 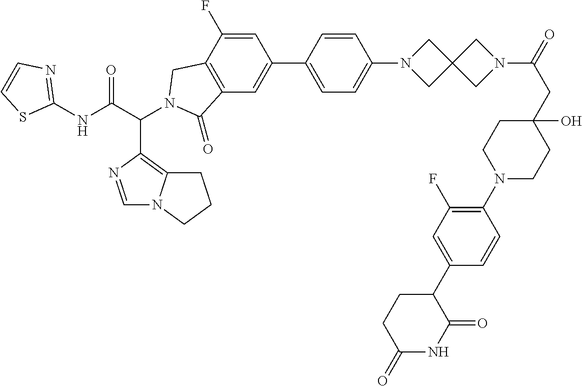 [SMILES: O=C1CCC(c2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | * | | |
| | 2-(6,7-dihydro-5H-pyrrolo[1,2-c]imidazol-1-yl)-2-(6-(4-(6- | | | |
| | (2-(1-(4-(2,6-dioxopiperidin-3-yl)-2-fluorophenyl)-4- | | | |
| | hydroxypiperidin-4-yl)acetyl)-2,6-diazaspiro[3.3]heptan-2- | | | |
| | yl)phenyl)-4-fluoro-1-oxoisoindolin-2-yl)-N-(thiazol-2- | | | |
| | yl)acetamide | | | |
| 159 | 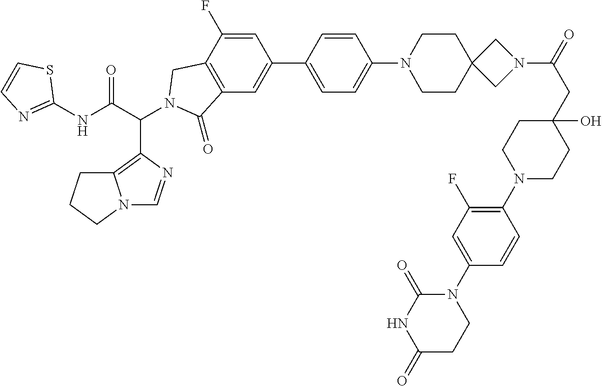 [SMILES: O=C1CCN(c2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1] | ** | | |
| | 2-(6,7-dihydro-5H-pyrrolo[1,2-c]imidazol-1-yl)-2-[6-[4-[2- | | | |
| | [2-[1-[4-(2,6-dioxo-3-piperidyl)-2-fluoro-phenyl]-4-hydroxy- | | | |
| | 4-piperidyl]acetyl]-2,7-diazaspiro[3.5]nonan-7-yl]phenyl]-4- | | | |
| | fluoro-1-oxo-isoindolin-2-yl]-N-thiazol-2-yl-acetamide | | | |
| 160 | 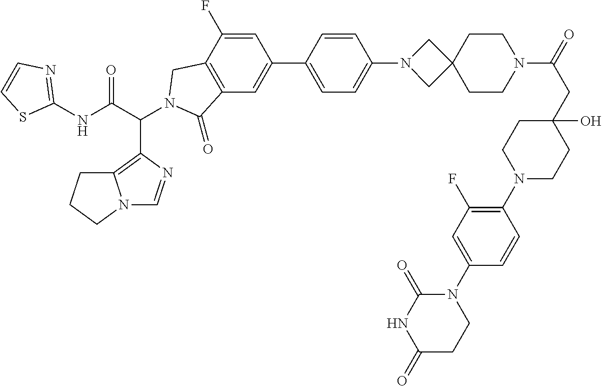 [SMILES: O=C1CCN(c2ccc(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | ** | | |
| | 2-(6,7-dihydro-5H-pyrrolo[1,2-c]imidazol-1-yl)-2-[6-[4-[7- | | | |
| | [2-[1-[4-(2,6-dioxo-3-piperidyl)-2-fluoro-phenyl]-4-hydroxy- | | | |
| | 4-piperidyl]acetyl]-2,7-diazaspiro[3.5]nonan-2-yl]phenyl]-4- | | | |
| | fluoro-1-oxo-isoindolin-2-yl]-N-thiazol-2-yl-acetamide | | | |
| 161 | 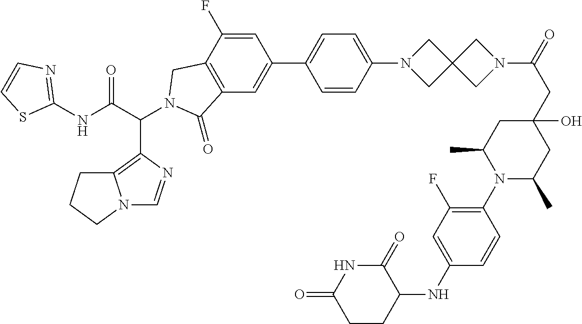 [SMILES: C[C@@H]1CC(O)(CC(=O)N2CC3(C2)CN(c2ccc(-c4cc(F)c5c(c4)C(=O)N(C(C(=O)Nc4nccs4)c4ncn6c4CCC6)C5)cc2)C3)C[C@H](C)N1c1ccc(NC2CCC(=O)NC2=O)cc1F] | *** | | |
| | 2-(6,7-dihydro-5H-pyrrolo[1,2-c]imidazol-1-yl)-2-[6-[4-[2- | | | |
| | [2-[(2s,6r)-1-[4-[(2,6-dioxo-3-piperidyl)amino]-2-fluoro- | | | |
| | phenyl]-4-hydroxy-2,6-dimethyl-4-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide | | | |
| 162 | 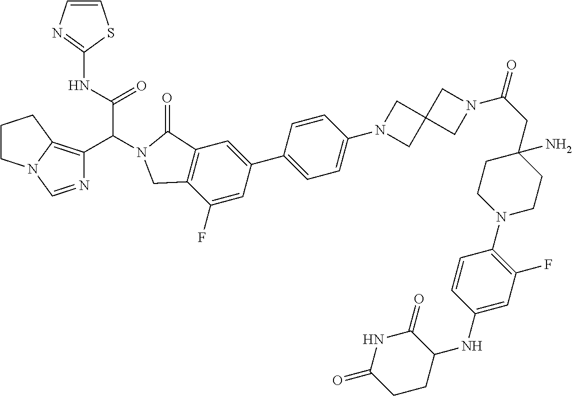 [SMILES: NC1(CC(=O)N2CC3(C2)CN(c2ccc(-c4cc(F)c5c(c4)C(=O)N(C(C(=O)Nc4nccs4)c4ncn6c4CCC6)C5)cc2)C3)CCN(c2ccc(NC3CCC(=O)NC3=O)cc2F)CC1] | *** | | |
| | 2-[6-[4-[2-[2-[4-amino-1-[4-[(2,6-dioxo-3-piperidyl)amino]- | | | |
| | 2-fluoro-phenyl]-4-piperidyl]acetyl]-2,6- | | | |
| | diazaspiro[3.3]heptan-6-yl]phenyl]-4-fluoro-1-oxo- | | | |
| | isoindolin-2-yl]-2-(6,7-dihydro-5H-pyrrolo[1,2-c]imidazol-1- | | | |
| | yl)-N-thiazol-2-yl-acetamide | | | |
| 163 | 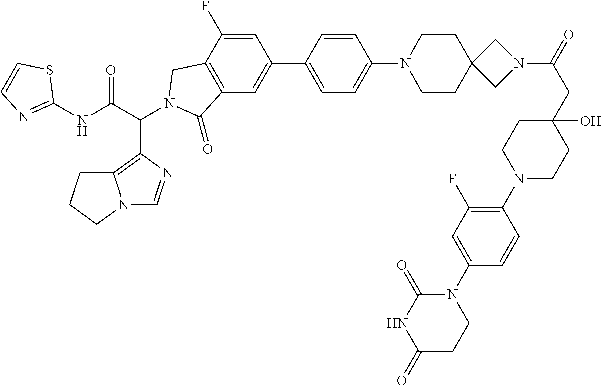 [SMILES: O=C1CCN(c2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(F)c2)C(=O)N1] | *** | | |
| | 2-(6,7-dihydro-5H-pyrrolo[1,2-c]imidazol-1-yl)-2-[6-[4-[2- | | | |
| | [2-[1-[4-(2,4-dioxohexahydropyrimidin-1-yl)-2-fluoro- | | | |
| | phenyl]-4-hydroxy-4-piperidyl]acetyl]-2,7- | | | |
| | diazaspiro[3.5]nonan-7-yl]phenyl]-4-fluoro-1-oxo- | | | |
| | isoindolin-2-yl]-N-thiazol-2-yl-acetamide | | | |
| 164 | 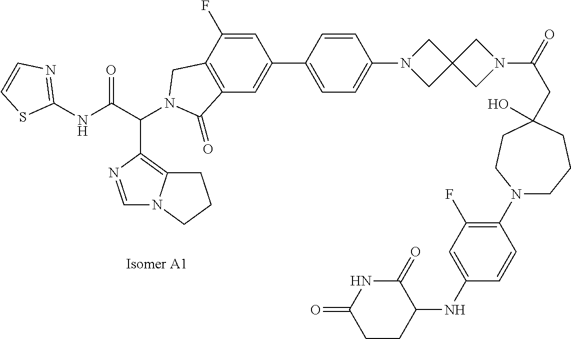 [SMILES: O=C1CCC(Nc2ccc(N3CCCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | ** | | |
| | 2-(6,7-dihydro-5H-pyrrolo[1,2-c]imidazol-1-yl)-2-[6-[4-[2- | | | |
| | [2-[1-[4-[[2,6-dioxo-3-piperidyl]amino]-2-fluoro-phenyl]-4- | | | |
| | hydroxy-azepan-4-yl]acetyl]-2,6-diazaspiro[3.3]heptan-6- | | | |
| | yl]phenyl]-4-fluoro-1-oxo-isoindolin-2-yl]-N-thiazol-2-yl- | | | |
| | acetamide, isomer A1 | | | |
| 165 | 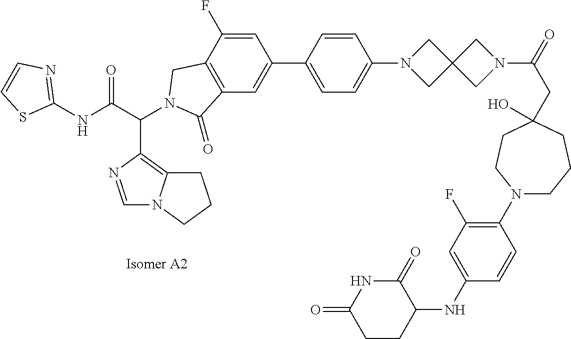 [SMILES: O=C1CCC(Nc2ccc(N3CCCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | *** | | |
| | 2-(6,7-dihydro-5H-pyrrolo[1,2-c]imidazol-1-yl)-2-[6-[4-[2- | | | |
| | [2-[1-[4-[[2,6-dioxo-3-piperidyl]amino]-2-fluoro-phenyl]-4- | | | |
| | hydroxy-azepan-4-yl]acetyl]-2,6-diazaspiro[3.3]heptan-6- | | | |
| | yl]phenyl]-4-fluoro-1-oxo-isoindolin-2-yl]-N-thiazol-2-yl- | | | |
| | acetamide, isomer A2 | | | |
| 166 | 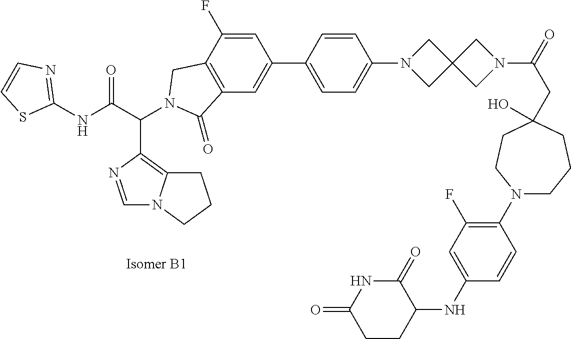 [SMILES: O=C1CCC(Nc2ccc(N3CCCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | ** | | |
| | 2-(6,7-dihydro-5H-pyrrolo[1,2-c]imidazol-1-yl)-2-[6-[4-[2- | | | |
| | [2-[1-[4-[[2,6-dioxo-3-piperidyl]amino]-2-fluoro-phenyl]-4- | | | |
| | hydroxy-azepan-4-yl]acetyl]-2,6-diazaspiro[3.3]heptan-6- | | | |
| | yl]phenyl]-4-fluoro-1-oxo-isoindolin-2-yl]-N-thiazol-2-yl- | | | |
| | acetamide, isomer B1 | | | |
| 167 | 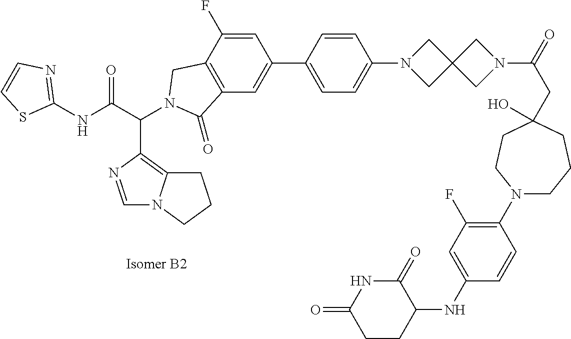 [SMILES: O=C1CCC(Nc2ccc(N3CCCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1] | ** | | |
| | 2-(6,7-dihydro-5H-pyrrolo[1,2-c]imidazol-1-yl)-2-[6-[4-[2- | | | |
| | [2-[1-[4-[[2,6-dioxo-3-piperidyl]amino]-2-fluoro-phenyl]-4- | | | |
| | hydroxy-azepan-4-yl]acetyl]-2,6-diazaspiro[3.3]heptan-6- | | | |
| | yl]phenyl]-4-fluoro-1-oxo-isoindolin-2-yl]-N-thiazol-2-yl- | | | |
| | acetamide, isomer B2 | | | |
| 168 | 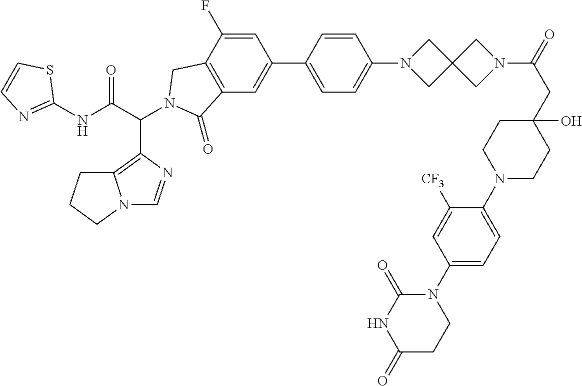 [SMILES: O=C1CCN(c2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(C(F)(F)F)c2)C(=O)N1] | ** | | |
| | 2-(6,7-dihydro-5H-pyrrolo[1,2-c]imidazol-1-yl)-2-[6-[4-[2- | | | |
| | [2-[1-[4-(2,4-dioxohexahydropyrimidin-1-yl)-2- | | | |
| | (trifluoromethyl)phenyl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide | | | |
| 169 | 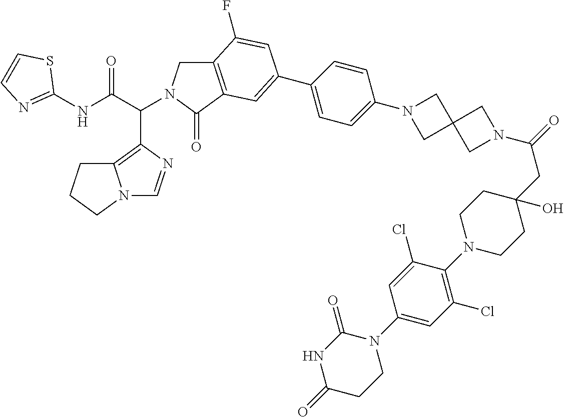 [SMILES: O=C1CCN(c2cc(Cl)c(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1] | *** | | |
| | 2-[6-[4-[2-[2-[1-[2,6-dichloro-4-(2,4- | | | |
| | dioxohexahydropyrimidin-1-yl)phenyl]-4-hydroxy-4- | | | |
| | piperidyl]acetyl]-2,6-diazaspiro[3.3]heptan-6-yl]phenyl]-4- | | | |
| | fluoro-1-oxo-isoindolin-2-yl]-2-(6,7-dihydro-5H-pyrrolo[1,2- | | | |
| | c]imidazol-1-yl)-N-thiazol-2-yl-acetamide | | | |
| 170 | 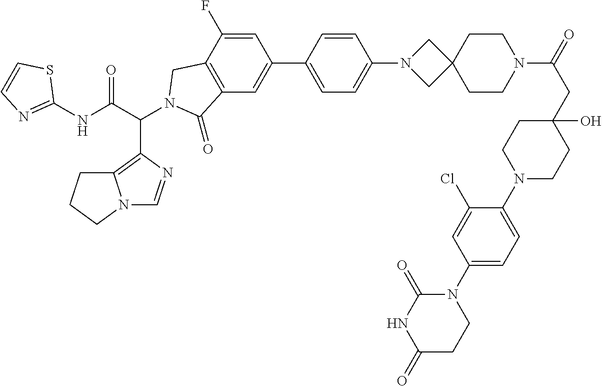 [SMILES: O=C1CCN(c2ccc(N3CCC(O)(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3)c(Cl)c2)C(=O)N1] | *** | | |
| | 2-[6-[4-[7-[2-[1-[2-chloro-4-(2,4-dioxohexahydropyrimidin- | | | |
| | 1-yl)phenyl]-4-hydroxy-4-piperidyl]acetyl]-2,7- | | | |
| | diazaspiro[3.5]nonan-2-yl]phenyl]-4-fluoro-1-oxo- | | | |
| | isoindolin-2-yl]-2-(6,7-dihydro-5H-pyrrolo[1,2-c]imidazol-1- | | | |
| | yl)-N-thiazol-2-yl-acetamide | | | |
| 171 | 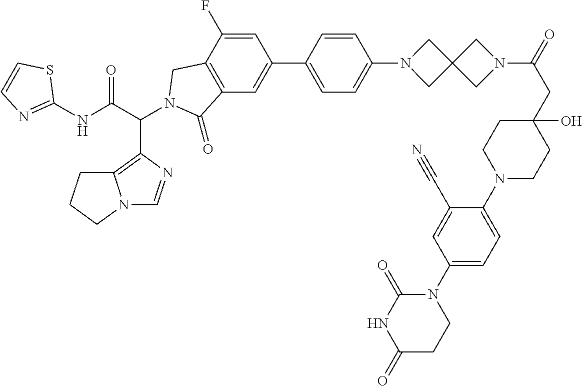 [SMILES: N#Cc1cc(N2CCC(=O)NC2=O)ccc1N1CCC(O)(CC(=O)N2CC3(C2)CN(c2ccc(-c4cc(F)c5c(c4)C(=O)N(C(C(=O)Nc4nccs4)c4ncn6c4CCC6)C5)cc2)C3)CC1] | * | | |
| | 2-[6-[4-[2-[2-[1-[2-cyano-4-(2,4-dioxohexahydropyrimidin- | | | |
| | 1-yl)phenyl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-2-(6,7-dihydro-5H-pyrrolo[1,2-c]imidazol-1- | | | |
| | yl)-N-thiazol-2-yl-acetamide | | | |
| 172 | 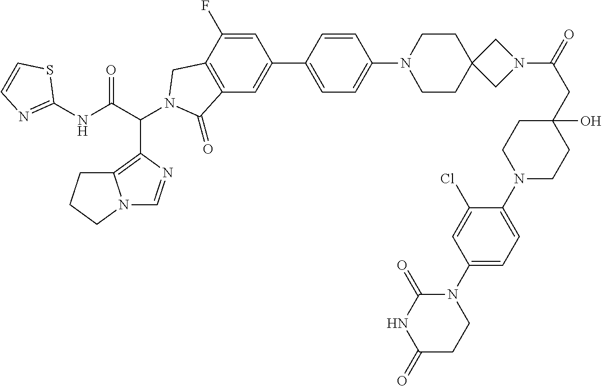 [SMILES: O=C1CCN(c2ccc(N3CCC(O)(CC(=O)N4CC5(CCN(c6ccc(-c7cc(F)c8c(c7)C(=O)N(C(C(=O)Nc7nccs7)c7ncn9c7CCC9)C8)cc6)CC5)C4)CC3)c(Cl)c2)C(=O)N1] | *** | | |
| | 2-[6-[4-[2-[2-[1-[2-chloro-4-(2,4-dioxohexahydropyrimidin- | | | |
| | 1-yl)phenyl]-4-hydroxy-4-piperidyl]acetyl]-2,7- | | | |
| | diazaspiro[3.5]nonan-7-yl]phenyl]-4-fluoro-1-oxo- | | | |
| | isoindolin-2-yl]-2-(6,7-dihydro-5H-pyrrolo[1,2-c]imidazol-1- | | | |
| | yl)-N-thiazol-2-yl-acetamide | | | |
| 173 | 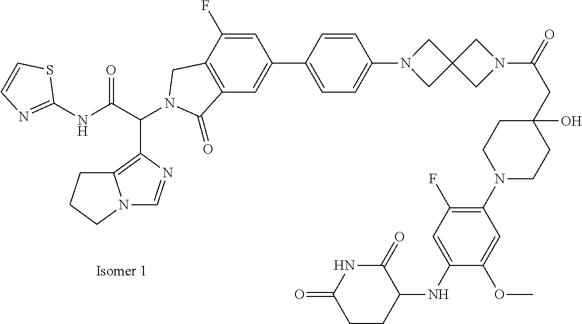 [SMILES: COc1cc(N2CCC(O)(CC(=O)N3CC4(C3)CN(c3ccc(-c5cc(F)c6c(c5)C(=O)N(C(C(=O)Nc5nccs5)c5ncn7c5CCC7)C6)cc3)C4)CC2)c(F)cc1NC1CCC(=O)NC1=O] | *** | | |
| | 2-(6,7-dihydro-5H-pyrrolo[1,2-c]imidazol-1-yl)-2-[6-[4-[2- | | | |
| | [2-[1-[4-[[2,6-dioxo-3-piperidyl]amino]-2-fluoro-5-methoxy- | | | |
| | phenyl]-4-hydroxy-4-piperidyl]acetyl]-2,6- | | | |
| | diazaspiro[3.3]heptan-6-yl]phenyl]-4-fluoro-1-oxo- | | | |
| | isoindolin-2-yl]-N-thiazol-2-yl-acetamide, isomer 1 | | | |
| 174 | 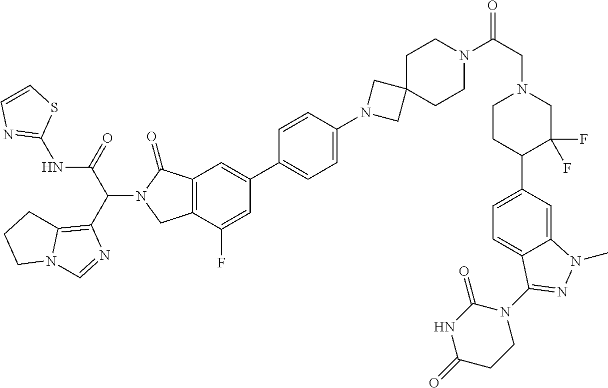 [SMILES: Cn1nc(N2CCC(=O)NC2=O)c2ccc(C3CCN(CC(=O)N4CCC5(CC4)CN(c4ccc(-c6cc(F)c7c(c6)C(=O)N(C(C(=O)Nc6nccs6)c6ncn8c6CCC8)C7)cc4)C5)CC3(F)F)cc21] | *** | | |
| | 2-(6,7-dihydro-5H-pyrrolo[1,2-c]imidazol-1-yl)-2-(6-(4-(7- | | | |
| | (2-(4-(3-(2,4-dioxotetrahydropyrimidin-1(2H)-yl)-1-methyl- | | | |
| | 1H-indazol-6-yl)-3,3-difluoropiperidin-1-yl)acetyl)-2,7- | | | |
| | diazaspiro[3.5]nonan-2-yl)phenyl)-4-fluoro-1-oxoisoindolin- | | | |
| | 2-yl)-N-(thiazol-2-yl)acetamide | | | |
| 175 | 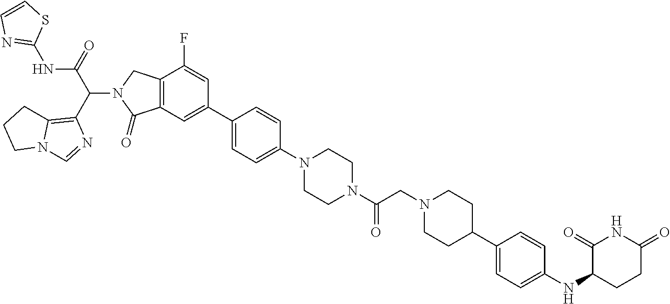 [SMILES: O=C1CC[C@@H](Nc2ccc(C3CCN(CC(=O)N4CCN(c5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | |
| | 2-(6,7-dihydro-5H-pyrrolo[1,2-c]imidazol-1-yl)-2-[6-[4-[4- | | | |
| | [2-[4-[4-[[(3R)-2,6-dioxo-3-piperidyl]amino]phenyl]-1- | | | |
| | piperidyl]acetyl]piperazin-1-yl]phenyl]-4-fluoro-1-oxo- | | | |
| | isoindolin-2-yl]-N-thiazol-2-yl-acetamide | | | |
| 176 | 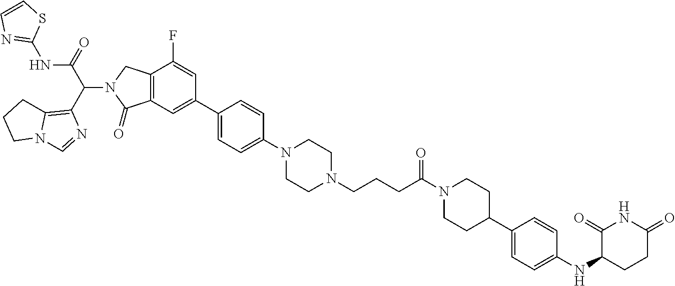 [SMILES: O=C1CC[C@@H](Nc2ccc(C3CCN(C(=O)CCCN4CCN(c5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | |
| | 2-(6,7-dihydro-5H-pyrrolo[1,2-c]imidazol-1-yl)-2-(6-(4-(4- | | | |
| | (4-(4-(4-((2,6-dioxopiperidin-3-yl)amino)phenyl)piperidin-1- | | | |
| | yl)-4-oxobutyl)piperazin-1-yl)phenyl)-4-fluoro-1- | | | |
| | oxoisoindolin-2-yl)-N-(thiazol-2-yl)acetamide | | | |
| 177 | 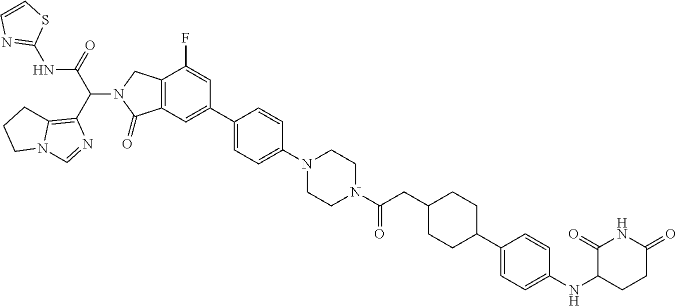 [SMILES: O=C1CCC(Nc2ccc(C3CCC(CC(=O)N4CCN(c5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | |
| | 2-(6,7-dihydro-5H-pyrrolo[1,2-c]imidazol-1-yl)-2-(6-(4-(4- | | | |
| | (2-(4-(4-((2,6-dioxopiperidin-3- | | | |
| | yl)amino)phenyl)cyclohexyl)acetyl)piperazin-1-yl)phenyl)-4- | | | |
| | fluoro-1-oxoisoindolin-2-yl)-N-(thiazol-2-yl)acetamide | | | |
| 178 | 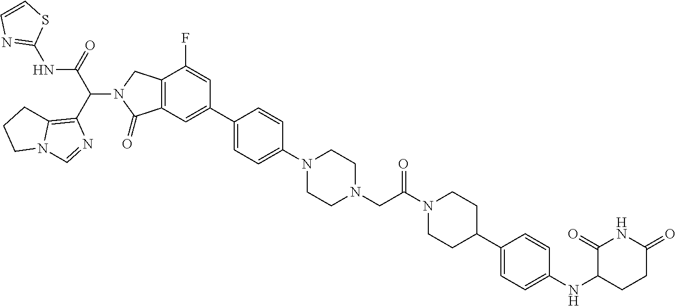 [SMILES: O=C1CCC(Nc2ccc(C3CCN(C(=O)CN4CCN(c5ccc(-c6cc(F)c7c(c6)C(=O)N(C(C(=O)Nc6nccs6)c6ncn8c6CCC8)C7)cc5)CC4)CC3)cc2)C(=O)N1] | *** | *** | |
| | 2-(6,7-dihydro-5H-pyrrolo[1,2-c]imidazol-1-yl)-2-(6-(4-(4- | | | |
| | (2-(4-(4-((2,6-dioxopiperidin-3-yl)amino)phenyl)piperidin-1- | | | |
| | yl)-2-oxoethyl)piperazin-1-yl)phenyl)-4-fluoro-1- | | | |
| | oxoisoindolin-2-yl)-N-(thiazol-2-yl)acetamide | | | |
| 179 | 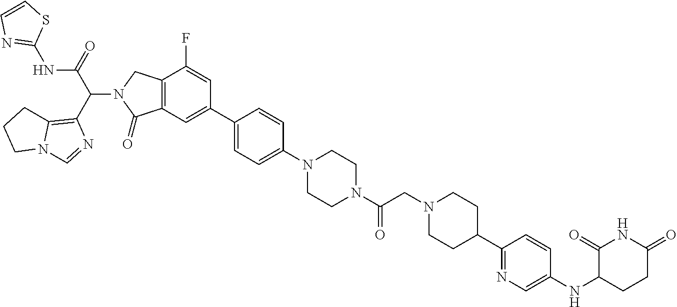 [SMILES: O=C1CCC(Nc2ccc(C3CCN(CC(=O)N4CCN(c5ccc(-c6cc(F)c7c(c6)C(=O)N(C(C(=O)Nc6nccs6)c6ncn8c6CCC8)C7)cc5)CC4)CC3)nc2)C(=O)N1] | *** | *** | |
| | 2-(6,7-dihydro-5H-pyrrolo[1,2-c]imidazol-1-yl)-2-[6-[4-[4- | | | |
| | [2-[4-[5-[(2,6-dioxo-3-piperidyl)amino]-2-pyridyl]-1- | | | |
| | piperidyl]acetyl]piperazin-1-yl]phenyl]-4-fluoro-1-oxo- | | | |
| | isoindolin-2-yl]-N-thiazol-2-yl-acetamide | | | |
| 180 | 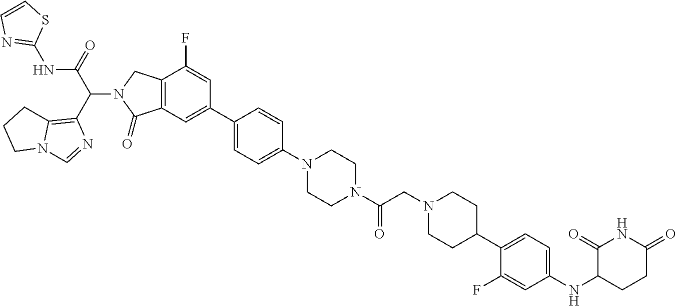 [SMILES: O=C1CCC(Nc2ccc(C3CCN(CC(=O)N4CCN(c5ccc(-c6cc(F)c7c(c6)C(=O)N(C(C(=O)Nc6nccs6)c6ncn8c6CCC8)C7)cc5)CC4)CC3)c(F)c2)C(=O)N1] | *** | *** | |
| | 2-(6,7-dihydro-5H-pyrrolo[1,2-c]imidazol-1-yl)-2-[6-[4-[4- | | | |
| | [2-[4-[4-[(2,6-dioxo-3-piperidyl)amino]-2-fluoro-phenyl]-1- | | | |
| | piperidyl]acetyl]piperazin-1-yl]phenyl]-4-fluoro-1-oxo- | | | |
| | isoindolin-2-yl]-N-thiazol-2-yl-acetamide | | | |

In the table above *** is <50 nM;
** is 50-150 nM; and
* is >150 nM

TABLE 10A
Degradation of Human Cancer Cell lines

| | | pEGFR | tEGFR |
|---|---|---|---|
| Cmpd | | IC₅₀ | DC₅₀ |
| # | Cell line | (nM) | (nM) |
| 1 | A431 (wild-type) | >10000 | >10000 |
| | H3255 (L858R) | 5 | 5 |
| | H1975 (L858R-T790M) | 9 | 8 |
| | H1975 + CS (L858R-T790M-C797S) | 10 | 12 |
| 2 | A431 (wild-type) | >10000 | >10000 |
| | H3255 (L858R) | 8 | 6 |
| | H1975 (L858R-T790M) | 14 | 12 |
| | H1975 + CS (L858R-T790M-C797S) | 29 | 19 |
| 3 | A431 (wild-type) | | >10000 |
| | H3255 (L858R) | 8 | 8 |
| | H1975 (L858R-T790M) | 11 | 10 |
| | H1975 + CS (L858R-T790M-C797S) | 19 | 33 |
| 4 | A431 (wild-type) | | |
| | H3255 (L858R) | 16 | 17 |
| | H1975 (L858R-T790M) | 15 | 15 |
| | H1975 + CS (L858R-T790M-C797S) | 22 | 33 |
| 5 | A431 (wild-type) | | >10000 |
| | H3255 (L858R) | 19 | 22 |
| | H1975 (L858R-T790M) | 12 | 11 |
| | H1975 + CS (L858R-T790M-C797S) | 22 | 23 |
| 7 | A431 (wild-type) | | |
| | H3255 (L858R) | | |
| | H1975 (L858R-T790M) | 17 | 15 |
| | H1975 + CS (L858R-T790M-C797S) | | |
| 8 | A431 (wild-type) | | |
| | H3255 (L858R) | | |
| | H1975 (L858R-T790M) | 22 | 23 |
| | H1975 + CS (L858R-T790M-C797S) | | |
| 9 | A431 (wild-type) | | |
| | H3255 (L858R) | | |
| | H1975 (L858R-T790M) | 30 | 24 |
| | H1975 + CS (L858R-T790M-C797S) | | |
| 10 | A431 (wild-type) | | |
| | H3255 (L858R) | | |
| | H1975 (L858R-T790M) | 39 | 38 |
| | H1975 + CS (L858R-T790M-C797S) | | |
| 11 | A431 (wild-type) | | |
| | H3255 (L858R) | | |
| | H1975 (L858R-T790M) | 22 | 20 |
| | H1975 + CS (L858R-T790M-C797S) | | |
| 12 | A431 (wild-type) | | |
| | H3255 (L858R) | | |
| | H1975 (L858R-T790M) | 20 | 15 |
| | H1975 + CS (L858R-T790M-C797S) | | |

TABLE 10B
Growth Inhibition of Ba/F3 Cell
Lines expressing EGFR variant

| Cmpd | | CTGlo |
|---|---|---|
| # | Cell line | (GI₅₀) nM |
| 1 | WT | 1619 |
| | L858R | 12 |
| | L858R-T790M | 9 |
| | L858R-C797S | 5 |
| | L858R-T790M-C797S | 7 |
| | L858R-L718Q | 24 |
| | L858R-T790M-L718Q | 44 |
| | L858R-L792H | 9 |
| | L858R-T790M-L792H | 21 |
| 2 | WT | 310 |
| | L858R | 3 |
| | L858R-T790M | 6 |
| | L858R-C797S | 3 |
| | L858R-T790M-C797S | 5 |
| | L858R-L718Q | 6 |
| | L858R-T790M-L718Q | 22 |
| | L858R-L792H | 4 |
| | L858R-T790M-L792H | 9 |
| 3 | WT | 248 |
| | L858R | 3 |
| | L858R-T790M | 9 |
| | L858R-C797S | 2 |
| | L858R-T790M-C797S | 3 |
| | L858R-L718Q | |
| | L858R-T790M-L718Q | |
| | L858R-L792H | |
| | L858R-T790M-L792H | |
| 7 | WT | |
| | L858R | |
| | L858R-T790M | |
| | L858R-C797S | |
| | L858R-T790M-C797S | 10 |
| | L858R-L718Q | 17 |
| | L858R-T790M-L718Q | 45 |
| | L858R-L792H | 7 |
| | L858R-T790M-L792H | 23 |
| 8 | WT | |
| | L858R | |
| | L858R-T790M | |
| | L858R-C797S | |
| | L858R-T790M-C797S | 13 |
| | L858R-L718Q | 2016 |
| | L858R-T790M-L718Q | 403 |
| | L858R-L792H | 213 |
| | L858R-T790M-L792H | 45 |
| 9 | WT | |
| | L858R | |
| | L858R-T790M | |
| | L858R-C797S | |
| | L858R-T790M-C797S | 16 |
| | L858R-L718Q | 23 |
| | L858R-T790M-L718Q | 82 |
| | L858R-L792H | 10 |
| | L858R-T790M-L792H | 33 |
| 11 | WT | |
| | L858R | |
| | L858R-T790M | |
| | L858R-C797S | |
| | L858R-T790M-C797S | 11 |
| | L858R-L718Q | 22 |
| | L858R-T790M-L718Q | 50 |
| | L858R-L792H | 9 |
| | L858R-T790M-L792H | 22 |

### IX. Synthetic Methods

The compounds of Formula I, II, III, or IV may contain one or more asymmetric centers and can therefore occur as racemates, racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within this invention. The present invention is meant to encompass all such isomeric forms of these compounds. The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography.

In the embodiments, where optically pure enantiomers are provided, optically pure enantiomer means that the compound contains >90% of the desired isomer by weight, particularly >95% of the desired isomer by weight, or more particularly >99% of the desired isomer by weight, said weight percent based upon the total weight of the isomer(s) of the compound. Chirally pure or chirally enriched compounds may be prepared by chirally selective synthesis or by separation of enantiomers. The separation of enantiomers may be carried out on the final product or alternatively on a suitable intermediate.

The preparation of compounds of Formula I is further described in more detail in the scheme below.

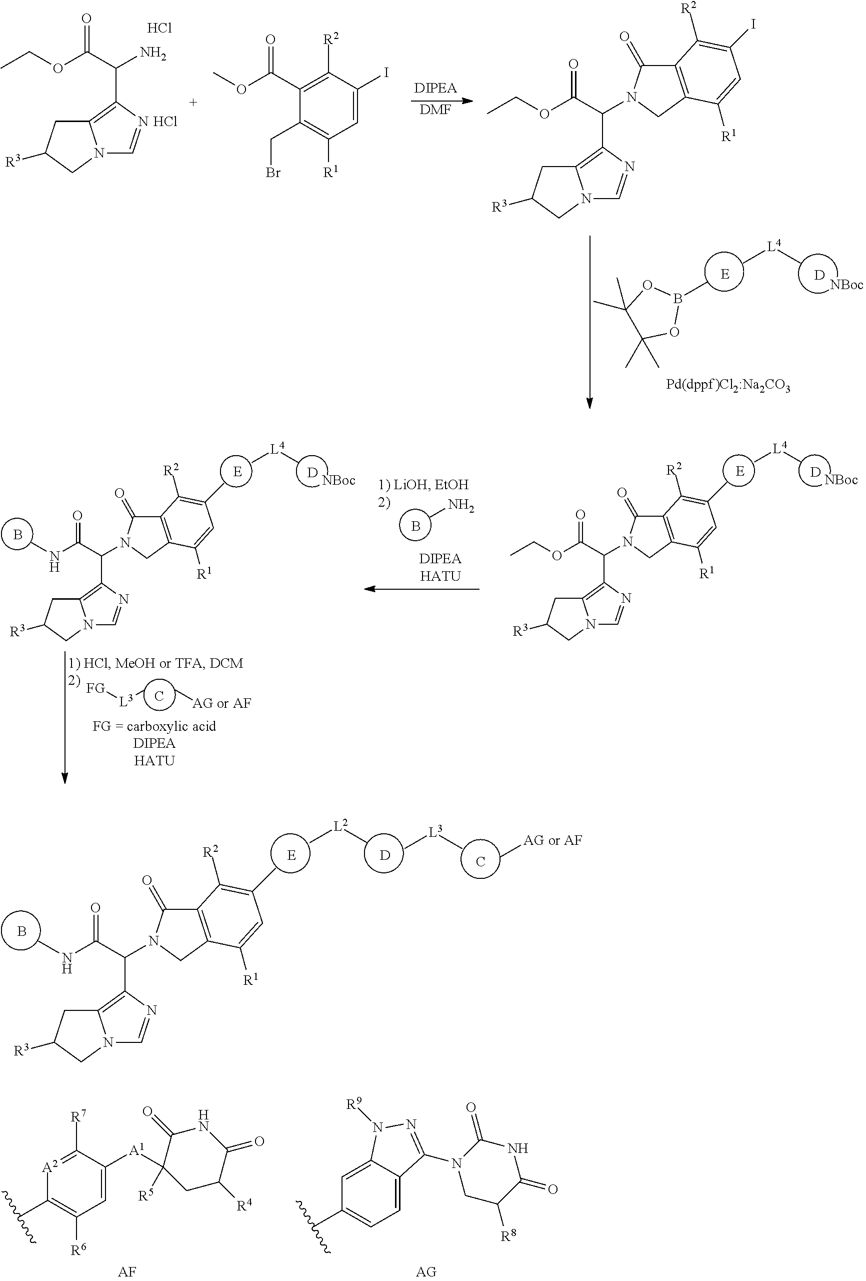

Generally speaking, the sequence of steps used to synthesize the compounds of Formula I can also be modified in certain cases.

The preparation of compounds of Formula II is further described in more detail in the scheme below.

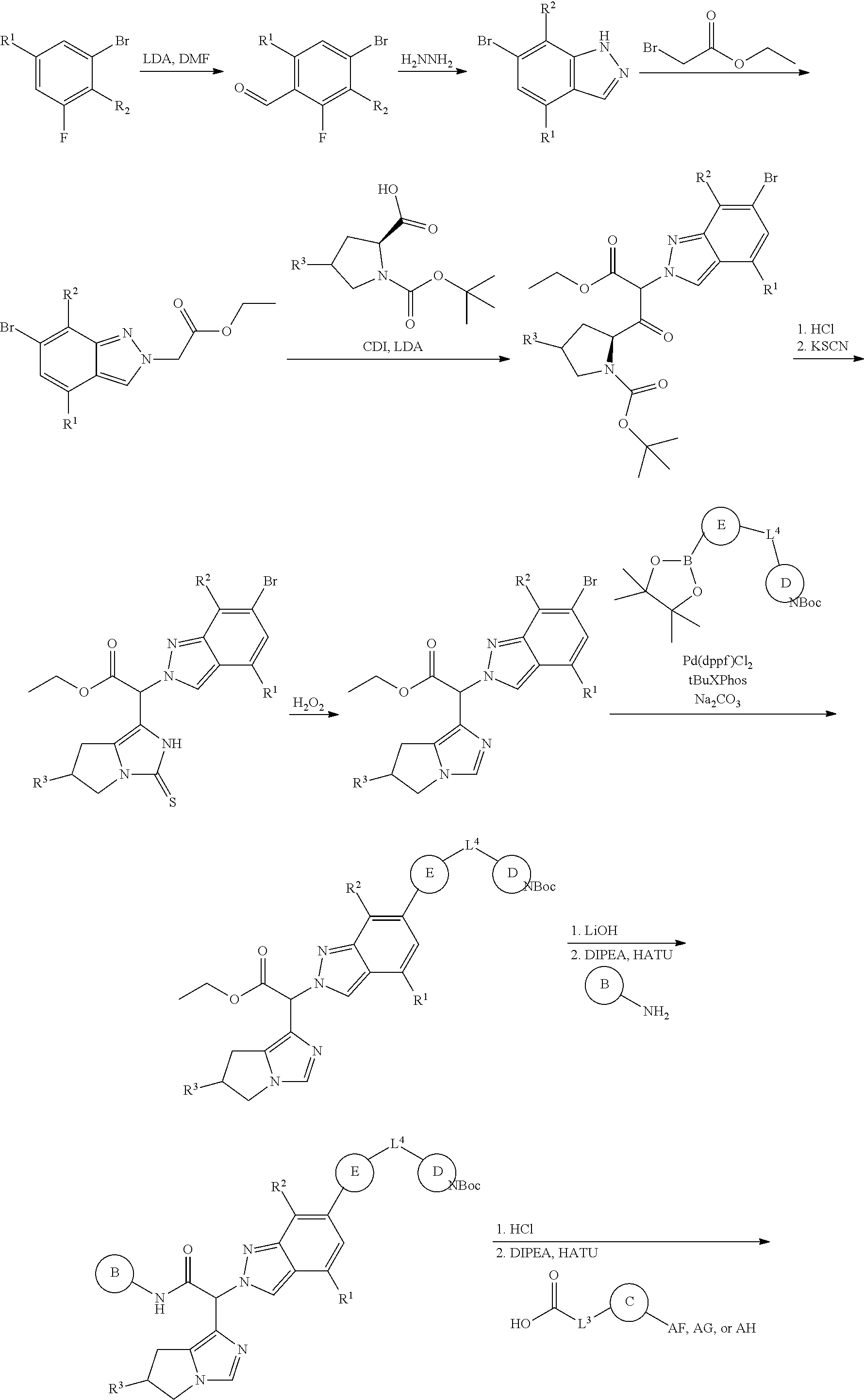

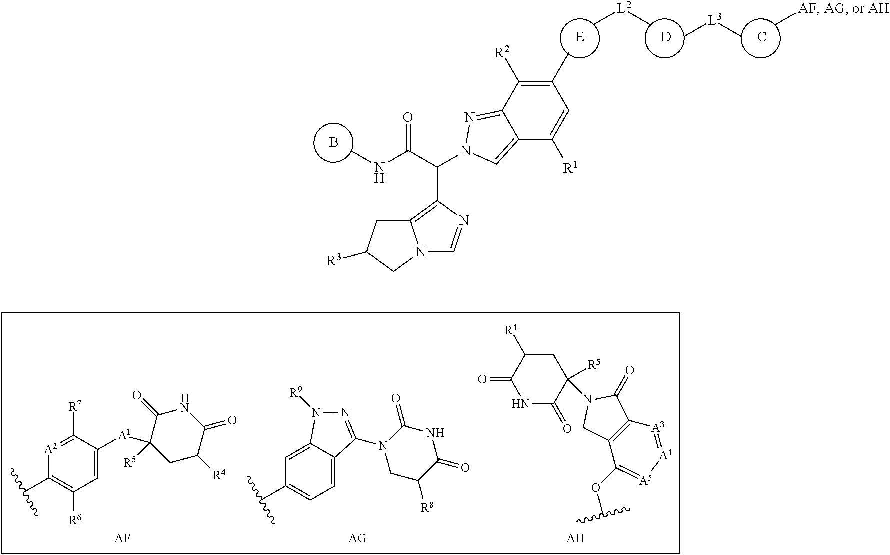

Generally speaking, the sequence of steps used to synthesize the compounds of Formula I can also be modified in certain cases. In certain cases the sequences of steps shown for Formula I or Formula II can be applied or modified for the synthesis of a compound of Formula III and Formula IV.

Isolation and Purification of the Compounds

Isolation and purification of the compounds and intermediates described herein can be effected, if desired, by any suitable separation or purification procedure such as, for example, filtration, extraction, crystallization, column chromatography, thin-layer chromatography, thick-layer chromatography, preparative low or high-pressure liquid chromatography or a combination of these procedures. Specific illustrations of suitable separation and isolation procedures can be had by reference to the preparations and examples herein below. However, other equivalent separation or isolation procedures could, of course, also be used. Racemic mixtures of chiral compounds of Formula I, II, III, or IV can be separated using chiral HPLC. Racemic mixtures of chiral synthetic intermediates may also be separated using chiral HPLC.

Salts of Compounds of Formula I, II, III, or IV

In cases where the compounds of Formula I, II, III, or IV are basic they may be converted to a corresponding acid addition salt. The conversion is accomplished by treatment with at least a stoichiometric amount of an appropriate acid, such as hydrochloric acid, hydrobromic acid, sulfuric acid, nitric acid, phosphoric acid and the like, and organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid and the like. A specific salt is the fumarate. Typically, the free base is dissolved in an inert organic solvent such as diethyl ether, ethyl acetate, chloroform, ethanol or methanol and the like, and the acid added in a similar solvent. The temperature is maintained between 0° C. and 50° C. The resulting salt precipitates spontaneously or may be brought out of solution with a less polar solvent.

Insofar as their preparation is not described in the examples, the compounds of Formula I, II, III, or IV as well as all intermediate products can be prepared according to analogous methods or according to the methods set forth herein. Starting materials are commercially available, known in the art or can be prepared by methods known in the art or in analogy thereto.

It will be appreciated that the compounds of general Formula I, II, III, or IV in this invention may be derivatised at functional groups to provide derivatives which are capable of conversion back to the parent compound in vivo.

### X. Experimental Procedures

Abbreviations

| | |
|---|---|
| ABPR | Automated back pressure regulator |
| AcCl | Acetyl Chloride |
| ACN | Acetonitrile |
| AIBN | Azobisisobutyronitrile |
| AlCl₃ | Aluminum trichloride |
| Ag₂CO₃ | Silver carbonate |
| Aq. | aqueous |
| AcOH | Acetic acid |
| BBr₃ | tribromoborane |
| B₂pin₂ | Bis(pinacolato)diboron |
| BINAP | 2,2′-bis(diphenylphosphino)-1,1′-binaphthyl |
| BnBr | Benzyl bromide |
| Boc₂O | Di-tert-butyl dicarbonate |
| Br₂ | Bromine gas |
| ClCOOEt | Ethyl chloroformate |
| CAN | Ceric ammonium nitrate |
| CDI | Carbonyldiimidazole |
| CuI | Cuprous iodide |
| CCl₄ | Carbon tetrachloride |
| CoCl₂ | Cobalt (II) chloride |
| CO | Carbon monoxide |
| CO₂ | Carbon dioxide |
| COMU | (1-Cyano-2-ethoxy-2-oxoethylidenaminooxy)dimethylamino- |
| | morpholino-carbenium hexafluorophosphate |
| Cu | copper |
| CH₃CN | Acetonitrile |
| CHCl₃ | Chloroform; trichloromethane |
| CH₂Cl₂, | Methylene chloride; dichloromethane |
| DCM | |
| Cs₂CO₃ | Cesium carbonate |
| CsF | Cesium fluoride |
| DAST | Diethylaminosulfur trifluoride |
| DBU | 1,8-Diazabicyclo[5.4.0]undec-7-ene |
| DCE | Dichloroethane; ethylene chloride |
| DIAD | Diisopropyl azodicarboxylate |
| DIEA, | N,N-diisopropylethylamine |
| DIPEA | |
| DMA; DMAC | N,N-dimethylacetamide |
| DMAP | 4-Dimethylaminopyridine |
| DMF | N,N-dimethylformamide |
| DMSO | Dimethylsulfoxide |
| DPPP | 1,3-Bis(diphenylphosphino)propane |
| EDCI | 1-Ethyl-3-(3-dimethylaminopropyl)carbodiimide |
| Et₃SiH | Triethylsilane |
| EtOAc; EA | Ethyl acetate |
| EtOH | Ethanol |
| FeBr₃ | Iron (III) bromide |
| HATU | 1-[Bis(dimethylamino)methylene]-1H-1,2,3-triazolo[4,5- |
| | b]pyridinium 3-oxide hexafluorophosphate |
| HCl | Hydrochloric acid |
| HCOONH₄ | Ammonium formate |
| H₂ | hydrogen gas |
| H₂O | Water |
| H₂O₂ | Hydrogen peroxide |
| HCOOH | Formic acid |
| H₃PO₂ | Hypophosphorous acid |
| H₂SO₄ | Sulfuric acid |
| HOBt | hydroxybenzotriazole |
| InCl₃ | Indium(III) chloride |
| IPA | Isopropyl alcohol |
| KHCO₃ | Potassium bicarbonate |
| KOAc | Potassium acetate |
| KOH | Potassium hydroxide |
| KO^{t}Bu | Potassium tert-butoxide |
| K₂CO₃ | Potassium carbonate |
| KHSO₄ | Potassium bisulfate |
| KI | Potassium iodide |
| KOCN | Potassium cyanate |
| KMnO₄ | Potassium permanganate |
| K₃PO₄ | Tribasic potassium phosphate |
| KSCN | Potassium thiocyanate |
| Lac | Lactic acid |
| LCMS | Liquid chromatography-mass spectrometry |
| LiAlH₄ | Lithium aluminum hydride |
| LiHMDS | Lithium bis(trimethylsilyl)amide |
| LiOH | Lithium hydroxide |
| LDA | Lithium diisopropylamide |
| Me | Methyl |
| MeCN | acetonitrile |
| MeI | Methyl iodide |
| MeOH | Methanol |
| MnO₂ | Manganese dioxide |
| MgCl₂ | Magnesium chloride |
| MgSO₄ | Magnesium sulfate |
| MsCl | Methanesulfonylchloride |
| MTBE | Methyl tert-butyl ether |
| NH₄OAc | Ammonium acetate |
| N₂ | Nitrogen gas |
| NaCN | Sodium cyanide |
| NH₄Cl | Ammonium chloride |
| NH₄OH | Ammonium hydroxide |
| NH₂OH | Hydroxylamine |
| NaBH(OAc)₃ | Sodium triacetoxyborohydride |
| NaBH₄ | Sodium borohydride |
| NaClO₂ | Sodium chlorite |
| Na₂SO₄ | Sodium sulfate |
| NaSMe | Sodium methanethiolate |
| Na₂S₂O₃ | Sodium thiosulfate |
| NaH | Sodium hydride |
| NaOH | Sodium hydroxide |
| NaHCO₃ | Sodium bicarbonate |
| Na₂CO₃ | Sodium carbonate |
| NaNO₂ | Sodium nitrite |
| NaOⁱPr | Sodium isopropoxide |
| NaH₂PO₄ | Sodium dihydrogen phosphate |
| NaIO₄ | Sodium periodate |
| NBS | N-bromo succinimide |
| NH₃ | Ammonia |
| N₂H₂ | Diazene |
| N₂H₄ | Hydrazine |
| NaNO₂ | Sodium nitrite |
| NMP | N-Methyl-2-pyrrolidone |
| OsO₄ | Osmium tetroxide |
| Pd(OAc)₂ | Palladium(II) acetate |
| Pd(PPh₃)₂Cl₂ | Bis(triphenylphosphine)palladium(II) dichloride |
| Pd(PPh₃)₄ | Tetrakis(triphenylphosphine)palladium(0) |
| Pd(dppf)Cl₂ | [1,1′-Bis(diphenylphosphino)ferrocene]dichloropalladium(II) |
| Pd/C | Palladium on carbon |
| Pd₂(dba)₃ | Tris(dibenzylideneacetone)dipalladium(0) |
| PhMe | toluene |
| PhNTF₂ | Bis(trifluoromethanesulfonyl)aniline |
| PPh₃ | Triphenylphosphine |
| P(pMeOPh)₃ | Tris(4-methoxyphenyl)phosphine |
| PCy₃ | Tricyclohexylphosphine |
| PET ether | Petroleum ether |
| PMB | Para-methoxybenzyl |
| POBr₃ | Phosphoryl bromide; phosphorus oxybromide |
| POCl₃ | Phosphoryl chloride; phosphorus oxychloride |
| PivOH | Pivalic acid |
| PtBu₃ | tritertbutylphosphine |
| PtO₂ | Platinum oxide |
| PTSA | p-toluenesulfonic acid |
| Py, py | Pyridine |
| PyBOP | (Benzotriazol-1-yloxy)tripyrrolidinophosphonium |
| | hexafluorophosphate |
| RBF | Round-bottom flask |
| RP | Reverse phase |
| rpm | Revolutions per minute |
| RT, rt | Room temperature |
| RuPhos | 2-Dicyclohexylphosphino-2′,6′-diisopropoxybiphenyl |
| SFC | Supercritical fluid chromatography |
| SOCl₂ | Thionyl chloride |
| T₃P | 1-Propanephosphonic anhydride |
| TBAB | Tetrabutylammonium bromide |
| TEA | Triethylamine |
| ^{t}BuBrettPhos | 2-(Di-tert-butylphosphino)-2′,4′,6′-triisopropyl-3,6- |
| | dimethoxy-1,1′-biphenyl |
| ^{t}BuOH | Tert-butyl alcohol |
| ^{t}BuONO | Tert-butyl nitrite |
| ^{t}BuXPhos | 2-Di-tert-butylphosphino-2′,4′,6′-triisopropylbiphenyl |
| ^{t}BuXPhos Pd G3 | 3^{rd} generation ^{t}BuXPhos palladium precatalyst |
| TBAI | Tetrabutylammonium iodide |
| TBTU | 2-(1H-Benzotriazole-1-yl)-1,1,3,3-tetramethylaminium |
| | tetrafluoroborate |
| Tf2O | Triflic anhydide |
| TFA | Trifluoroacetic acid |
| TFBen | Benzene-1,3,5-triyl triformate |
| TMSCN | Trimethylsilyl cyanide |
| THF | Tetrahydrofuran |
| TLC | Thin layer chromatography |
| XantPhos | 4,5-Bis(diphenylphosphino)-9,9-dimethylxanthene |
| XPhos | 2-Dicyclohexylphosphino-2′,4′,6′-triisopropylbiphenyl |
| Zn | zinc |

### Synthetic Examples

The following examples are provided for illustration of the invention. They should not be considered as limiting the scope of the invention, but merely as being representative thereof.

### Intermediates

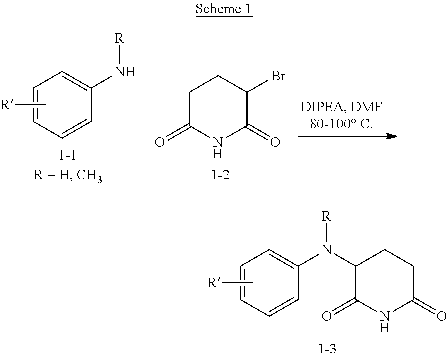

### General Procedure—A

To a mixture of 1-1 (1 mmol) and 1-2 (2 mmol) in dioxane (3 mL) was added N,N-Diisopropylethylamine (2 mmol). The resulting solution was heated in a sealed tube at 70-110° C. for 24 hours to produce 1-3. Reaction mixture was then cooled to room temperature, diluted with water and extracted with Ethyl acetate. The combined Ethyl acetate extract was washed with brine, dried over anhydrous Na₂SO₄ and concentrated under reduced pressure. The residue was purified by column chromatography (silica, gradient: 0-3% methanol in dichloromethane) to afford 1-3.

### Intermediate tert-butyl 4-(4-((2,6-dioxopiperidin-3-yl)amino)phenyl)piperidine-1-carboxylate

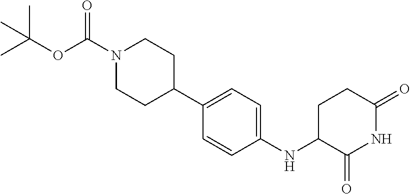 [SMILES: CC(C)(C)OC(=O)N1CCC(c2ccc(NC3CCC(=O)NC3=O)cc2)CC1]

tert-butyl 4-(4-((2,6-dioxopiperidin-3-yl)amino)phenyl)piperidine-1-carboxylate was synthesized from tert-Butyl 4-(4-aminophenyl)-1-piperidinecarboxylate (CAS #170011-57-1) following general procedure A (N,N-diisopropylethylamine/Dioxane). Yield-45%; ¹H NMR (400 MHz, DMSO-d₆) δ 10.75 (s, 1H), 6.94 (d, J=8.16 Hz, 2H), 6.60 (d, J=7.88 Hz, 2H), 5.64 (d, J=6.96 Hz, 1H), 4.28-4.24 (m, 1H), 4.07-4.00 (m, 2H), 2.79-2.64 (m, 4H), 2.53-2.48 (m, 2H), 2.11-2.05 (m, 1H), 1.89-1.81 (m, 1H), 1.71-1.64 (m, 2H0, 1.40-1.34 (m, 10H); LC MS: ES+ 386.3.

### Intermediate 3-((3-(piperidin-4-yl)phenyl)amino)piperidine-2,6-dione hydrochloride

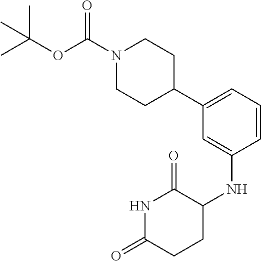 [SMILES: CC(C)(C)OC(=O)N1CCC(c2cccc(NC3CCC(=O)NC3=O)c2)CC1]

Tert-butyl 4-(3-((2,6-dioxopiperidin-3-yl)amino)phenyl)piperidine-1-carboxylate was synthesized from tert-Butyl 4-[3-aminophenyl]-1-piperidinecarboxylate (CAS #387827-19-2) following the general procedure A. Yield: 25% LCMS (ESI+): 388.2 (M+H)

### Intermediate 3-((6-(piperidin-4-yl)pyridin-3-yl)amino)piperidine-2,6-dione hydrochloride

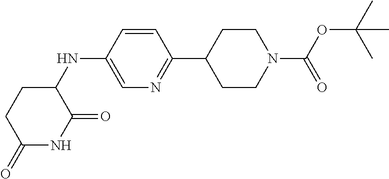 [SMILES: CC(C)(C)OC(=O)N1CCC(c2ccc(NC3CCC(=O)NC3=O)cn2)CC1]

Tert-butyl 4-(5-((2,6-dioxopiperidin-3-yl)amino)pyridin-2-yl)piperidine-1-carboxylate was synthesized from tert-butyl 4-(5-aminopyridin-2-yl)piperidine-1-carboxylate (CAS #885693-48-1) following the general procedure: Yield: 14%, LCMS (ESI+): 389.2 (M+H).

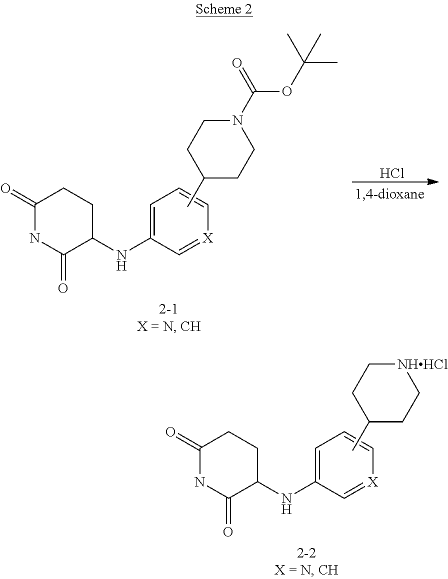

### General Procedure B:

To 2-1 dissolved in methanol (0.1 M) at room temperature was added hydrogen chloride (4M in 1,4-dioxane, 5 equiv.) and the reaction mixture was heated at 40° C. for 2 hours. The volatiles were evaporated under reduced pressure to afford 2-2.

### Intermediate 3-((4-(piperidin-4-yl)phenyl)amino)piperidine-2,6-dione hydrochloride

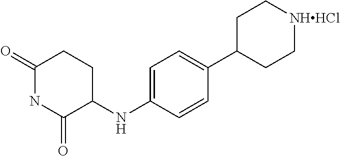

3-((4-(piperidin-4-yl)phenyl)amino)piperidine-2,6-dione hydrochloride was synthesized from tert-butyl 4-(4-((2,6-dioxopiperidin-3-yl)amino)phenyl)piperidine-1-carboxylate following general procedure (General procedure—B). Yield-88%; ¹H NMR (400 MHz, DMSO-d₆) δ 10.80 (s, 1H), 8.84 (brs, 1H), 8.77 (brs, 1H), 6.95 (d, J=8.44 Hz, 2H), 6.66 (d, J=8.48 Hz, 2H), 4.29 (dd, J=11.4, 4.72 Hz, 1H), 3.35-3.29 (m, 2H), 2.99-2.91 (m, 2H), 2.71-2.53 (m, 3H), 2.10-2.05 (m, 1H), 1.89-1.71 (m, 5H); LC MS: ES+ 288.2.

### Intermediate 3-((3-(piperidin-4-yl)phenyl)amino)piperidine-2,6-dione hydrochloride

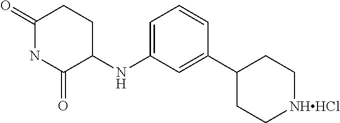

3-((3-(piperidin-4-yl)phenyl)amino)piperidine-2,6-dione hydrochloride was synthesized from tert-butyl 4-(3-((2,6-dioxopiperidin-3-yl)amino)phenyl)piperidine-1-carboxylate following the general procedure B. Yield: 76% ¹H NMR (400 MHz, DMSO-d₆) δ 10.80 (s, 1H), 9.00 (br.s, 1H), 8.85 (br. S, 1H), 1.02 (t, J=7.6 Hz, 1H), 6.57-6.55 (m, 2H), 6.47 (d, J=7.6 Hz, 1H), 4.32 (dd, J=11.2 Hz, 4.6 Hz, 1H), 3.45-3.39 (m, 2H), 2.80-2.65 (m, 2H), 2.79-2.67 (m, 2H), 2.61-2.53 (M, 1H), 2.11-2.07 (m, 1H), 1.94-1.80 (m, 5H). LCMS (ESI+): 288.2 (M+H).

### Intermediate 3-((6-(piperidin-4-yl)pyridin-3-yl)amino)piperidine-2,6-dione hydrochloride

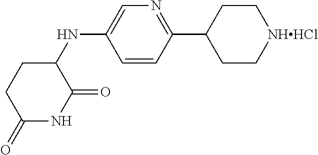

3-((6-(piperidin-4-yl)pyridin-3-yl)amino)piperidine-2,6-dione hydrochloride was synthesized from tert-butyl 4-(5-((2,6-dioxopiperidin-3-yl)amino)pyridin-2-yl)piperidine-1-carboxylate following the general procedure B. Yield: 83%, LCMS (ESI+): 289.0 (M+H).

### Synthesis of Intermediate tert-butyl 4-[4-[[(3S)-2,6-dioxo-3-piperidyl]amino]phenyl]piperidine-1-carboxylate and tert-butyl 4-[4-[[(3R)-2,6-dioxo-3-piperidyl]amino]phenyl]piperidine-1-carboxylate by chiral SFC separation

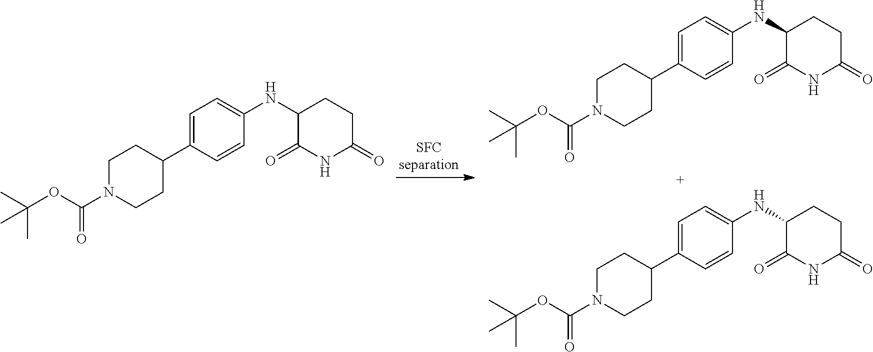 [SMILES: CC(C)(C)OC(=O)N1CCC(c2ccc(NC3CCC(=O)NC3=O)cc2)CC1 ; CC(C)(C)OC(=O)N1CCC(c2ccc(N[C@@H]3CCC(=O)NC3=O)cc2)CC1 ; CC(C)(C)OC(=O)N1CCC(c2ccc(N[C@H]3CCC(=O)NC3=O)cc2)CC1]

Separation of tert-butyl 4-[4-[(2,6-dioxo-3-piperidyl)amino]phenyl]piperidine-1-carboxylate (4 g, 10.32 mmol) by chiral SFC afforded two sets of fractions.

The following preparative scale SFC method was used to separate the enantiomers:
Column: Chiralpak ID (250×21 mm) 5 um
Flow: 35 g/min
Mobile Phase: 45% CO2+55% Isopropyl alcohol
ABPR: 100 bar
Temperature: 35° C.

The earlier eluting fractions were lyophilized to afford tert-butyl 4-[4-[[(3S)-2,6-dioxo-3-piperidyl]amino]phenyl]piperidine-1-carboxylate (1.44 g, 3.70 mmol, 35.88% yield, 99.66% enantiomeric excess, Chiral SFC Rt=4.31 min). ¹H NMR (400 MHz, DMSO-d₆) δ 10.77 (s, 1H), 6.94 (d, J=8.1 Hz, 2H), 6.60 (d, J=8.2 Hz, 2H), 5.68-5.66 (m, 1H), 4.29-4.23 (m, 1H), 4.05-4.02 (m, 2H), 2.78-2.54 (m, 5H), 2.11-2.07 (m, 1H), 1.89-1.83 (m, 1H), 1.69-1.66 (m, 2H), 1.40-1.36 (m 11H).

The later fractions were lyophilized to afford tert-butyl 4-[4-[[(3R)-2,6-dioxo-3-piperidyl]amino]phenyl]piperidine-1-carboxylate (1.56 g, 3.95 mmol, 38.24% yield, 98.06% enantiomeric excess, Chiral SFC Rt=5.96 min). ¹H NMR (400 MHz, DMSO-d₆) δ 10.77 (s, 1H), 6.94 (d, J=8.2 Hz, 2H), 6.60 (d, J=8.3 Hz, 2H), 5.68-5.66 (m, 1H), 4.29-4.23 (m, 1H), 4.05-4.02 (m, 2H), 2.78-2.58 (m, 5H), 2.11-2.07 (m, 1H), 1.87-1.83 (m, 1H), 1.70-1.67 (m, 2H), 1.40-1.35 (m 11H).

### Synthesis of 2-[4-[4-[[(3S)-2,6-dioxo-3-piperidyl]amino]phenyl]-1-piperidyl]acetic acid trifluoroacetic acid salt and 2-[4-[4-[[(3R)-2,6-dioxo-3-piperidyl]amino]phenyl]-1-piperidyl]acetic acid trifluoroacetic acid salt

### Tert-butyl 2-[4-[4-[(2,6-dioxo-3-piperidyl)amino]phenyl]-1-piperidyl]acetate

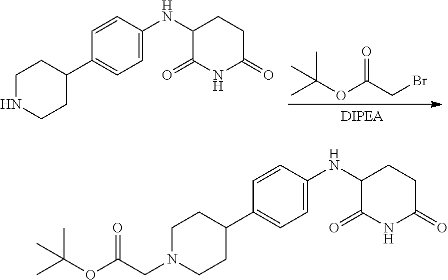

To a stirred solution of 3-[4-(4-piperidyl)anilino]piperidine-2,6-dione (2.0 g, 6.96 mmol) in DMF (20 mL) was added triethyl amine (3.52 g, 34.80 mmol, 4.85 mL) followed by tert-butyl 2-bromoacetate (1.49 g, 7.66 mmol, 1.12 mL) and stirred the reaction mixture at rt for 16 h. Water (75 mL) was added and the product was extracted with ethyl acetate (3×150 mL). The combined organic layers were dried over sodium sulphate, filtered and concentrated under reduced pressure.

The residue was purified by silica gel chromatography using 30% ethyl acetate-pet ether as eluent to give tert-butyl 2-[4-[4-[(2,6-dioxo-3-piperidyl)amino]phenyl]-1-piperidyl]acetate (1.40 g, 3.36 mmol, 48.33% yield) as a green solid.

### SFC Separation Conditions to Obtain Tert-butyl (S)-2-[4-[4-[(2,6-dioxo-3-piperidyl)amino]phenyl]-1-piperidyl]acetate and tert-butyl (R)-2-[4-[4-[(2,6-dioxo-3-piperidyl)amino]phenyl]-1-piperidyl]

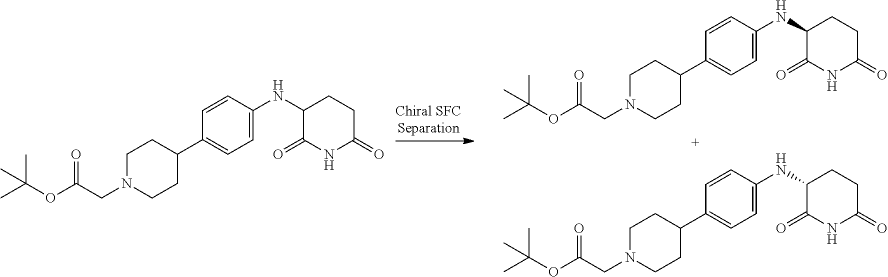 [SMILES: CC(C)(C)OC(=O)CN1CCC(c2ccc(NC3CCC(=O)NC3=O)cc2)CC1 ; CC(C)(C)OC(=O)CN1CCC(c2ccc(N[C@@H]3CCC(=O)NC3=O)cc2)CC1 ; CC(C)(C)OC(=O)CN1CCC(c2ccc(N[C@H]3CCC(=O)NC3=O)cc2)CC1]

The racemic intermediate tert-butyl 2-[4-[4-[(2,6-dioxo-3-piperidyl)amino]phenyl]-1-piperidyl]acetate (1.40 g, 3.36 mmol) was resolved using chiral SFC method using Chiralcel OD-H column (250 mm×30 mm; 5 micron) eluting with 40% isopropyl alcohol/CO2 (Flow Rate: 3 ml/min; Outlet Pressure: 100 bar). The first eluting set of fractions was evaporated under reduced pressure to afford tert-butyl (S)-2-[4-[4-[(2,6-dioxo-3-piperidyl)amino]phenyl]-1-piperidyl]acetate (500 mg, 36% yield, Rt=3.36 min, 96.22% purity, >99% enantiomeric excess). The second set of fractions was evaporated under reduced pressure to afford 500 mg of tert-butyl (R)-2-[4-[4-[(2,6-dioxo-3-piperidyl)amino]phenyl]-1-piperidyl]acetate (500 mg, 36% yield, Rt=4.84 min., purity 96.22%, 99.04% enantiomeric excess). LCMS First eluted (m/z: 402.4 [M+H]), LCMS Second eluted (m/z: 402.2 [M+H]).

### 2-[4-[4-[[(3S)-2,6-dioxo-3-piperidyl]amino]phenyl]-1-piperidyl]acetic acid trifluoroacetic acid salt

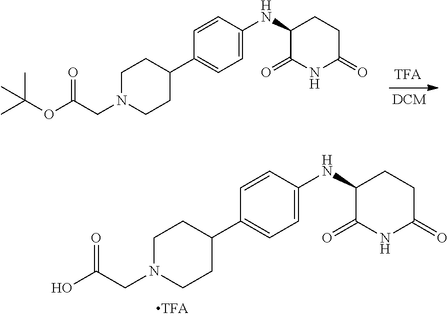 [SMILES: CC(C)(C)OC(=O)CN1CCC(c2ccc(N[C@H]3CCC(=O)NC3=O)cc2)CC1 ; O=C(O)CN1CCC(c2ccc(N[C@H]3CCC(=O)NC3=O)cc2)CC1]

tert-butyl 2-[4-[4-[[(3S)-2,6-dioxo-3-piperidyl]amino]phenyl]-1-piperidyl]acetate (500 mg, 1.25 mmol) was dissolved in dichloromethane (5 mL) and trifluoroacetic acid (12.26 g, 107.51 mmol, 8 mL) was added dropwise at 0° C. and the reaction was stirred at room temperature for 3 h. After completion of the reaction, reaction mixture was concentrated. The material was triturated with a methanol:MTBE mixture (1:4), solid was collected and the volatiles were evaporated under reduced pressure to give 2-[4-[4-[[(3S)-2,6-dioxo-3-piperidyl]amino]phenyl]-1-piperidyl]acetic acid trifluoroacetic acid salt (600 mg, 1.24 mmol, 99.6% yield) as an off white solid. LCMS (ESI+): 346.1 (M+H)

### 2-[4-[4-[[(3R)-2,6-dioxo-3-piperidyl]amino]phenyl]-1-piperidyl]acetic acid:trifluoroacetic acid salt

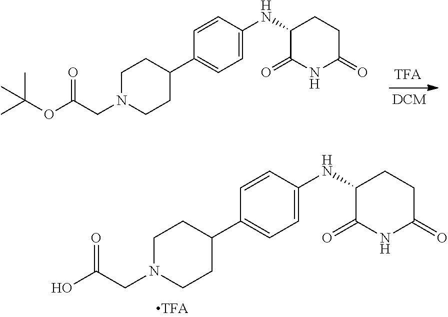 [SMILES: CC(C)(C)OC(=O)CN1CCC(c2ccc(N[C@@H]3CCC(=O)NC3=O)cc2)CC1 ; O=C(O)CN1CCC(c2ccc(N[C@@H]3CCC(=O)NC3=O)cc2)CC1]

tert-butyl 2-[4-[4-[[(3R)-2,6-dioxo-3-piperidyl]amino]phenyl]-1-piperidyl]acetate (500.00 mg, 1.25 mmol) was treated in a way similar to 2-[4-[4-[[(3S)-2,6-dioxo-3-piperidyl]amino]phenyl]-1-piperidyl]acetic acid trifluoroacetic acid salt to yield 2-[4-[4-[[(3R)-2,6-dioxo-3-piperidyl]amino]phenyl]-1-piperidyl]acetic acid trifluoroacetic acid salt (600 mg, 1.24 mmol, 99.63% yield) as an off white solid. LCMS (ESI+): 346.1 (M+H)

### Synthesis of Intermediate 3-(3-Fluoro-4-piperidin-4-yl-phenylamino)-piperidine-2,6-dione hydrochloride

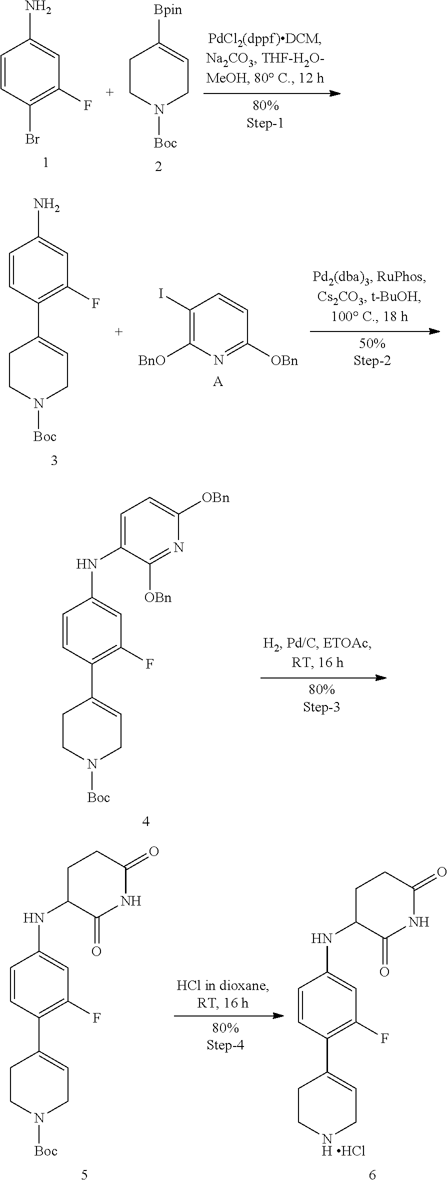

### Step-1: Preparation of 4-(4-Amino-2-fluoro-phenyl)-3, 6-dihydro-21I-pyridine-1-carboxylic acid tert-butyl ester

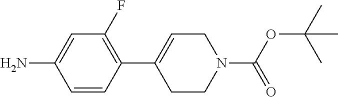 [SMILES: CC(C)(C)OC(=O)N1CC=C(c2ccc(N)cc2F)CC1]

Sodium carbonate (6.14 g, 57.89 mmol, 2.43 mL) was added to a stirred solution of 4-bromo-3-fluoro-aniline (5.00 g, 26.3 mmol) and tert-butyl 4-(4,4,5,5-tetramethyl-1,3,2-dioxaborolan-2-yl)-3,6-dihydro-2H-pyridine-1-carboxylate (8.95 g, 29.0 mmol) in water (12 mL), THF (60 mL) and methanol (24 mL) and the flask was thoroughly purged with argon. PdCl₂(dppf)·dichloromethane (430 mg, 526 μmol) was added and the reaction mixture was degassed with nitrogen and then heated at 80° C. for 12 h. The reaction mixture was diluted with ethyl acetate, filtered through a short pad of celite and washed with ethyl acetate. The combined organic extracts were washed with water and brine, dried over anhydrous sodium sulphate, filtered and concentrated under reduced pressure. The residue was purified by column chromatography (15% ethyl acetate-hexane) to get tert-butyl 4-(4-amino-2-fluoro-phenyl)-3,6-dihydro-2H-pyridine-1-carboxylate (6.1 g, 20.9 mmol, 79% yield) as pale yellow solid. LCMS: ESI+293 (M+Hs)

### Step-2: Preparation of 4-[4-(2,6-Bis-benzyloxy-pyridin-3-ylamino)-2-fluoro-phenyl]-3,6-dihydro-2H-pyridine-1-carboxylic acid tert-butyl ester

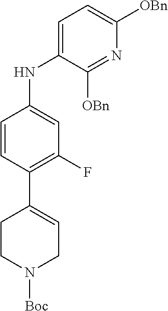 [SMILES: CC(C)(C)OC(=O)N1CC=C(c2ccc(Nc3ccc(OCc4ccccc4)nc3OCc3ccccc3)cc2F)CC1]

Cesium carbonate (19.73 g, 60.54 mmol) was added to a stirred solution of tert-butyl 4-(4-amino-2-fluoro-phenyl)-3,6-dihydro-2H-pyridine-1-carboxylate (5.9 g, 20.2 mmol) and 2,6-dibenzyloxy-3-iodo-pyridine (9.26 g, 22.2 mmol) in t-BuOH (60 mL) The resulting mixture was degassed with argon and Pd₂(dba)₃ (924 mg, 1.01 mmol), Ruphos (942 mg, 2.02 mmol) were added under inert atmosphere. The resulting mixture was heated at 100° C. for 18 h. The reaction mixture was diluted with ethyl acetate, filtered through a short pad of celite and washed with ethyl acetate. The combined organic extracts were washed with water and brine, dried over anhydrous sodium sulphate, filtered and concentrated under reduced pressure. The residue was purified by column chromatography (15% ethyl acetate-hexane) to get tert-butyl 4-[4-[(2,6-dibenzyloxy-3-pyridyl)amino]-2-fluoro-phenyl]-3,6-dihydro-2H-pyridine-1-carboxylate (5.9 g, 10.1 mmol, 50% yield) as pale yellow solid. LCMS: ES+ 582 (M+H⁺)

### Step-3: Preparation of 4-[4-(2,6-Dioxo-piperidin-3-ylamino)-2-fluoro-phenyl]-piperidine-1-carboxylic acid tert-butyl ester

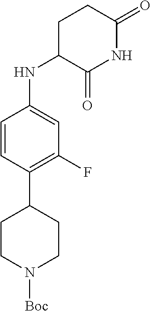 [SMILES: CC(C)(C)OC(=O)N1CCC(c2ccc(NC3CCC(=O)NC3=O)cc2F)CC1]

10% Pd—C (50% wet, 4.6 g) was added to a stirred nitrogen-degassed solution of tert-butyl 4-[4-[(2,6-dibenzyloxy-3-pyridyl)amino]-2-fluoro-phenyl]-3,6-dihydro-2H-pyridine-1-carboxylate (4.6 g, 7.91 mmol) in ethyl acetate (40 mL). The resulting mixture was stirred at ambient temperature under hydrogen balloon pressure for 20 h. The reaction mixture was filtered through a small pad of celite and washed with ethyl acetate. The combined filtrate was evaporated under reduced pressure and purified by column chromatography (40% ethyl acetate in hexane) to afford tert-butyl 4-[4-[(2,6-dioxo-3-piperidyl)amino]-2-fluoro-phenyl]piperidine-1-carboxylate (2.6 g, 6.41 mmol, 81% yield) as a blue solid. LCMS: ES+ 406 (M+H⁺).

### Step-4: Preparation of 3-(3-Fluoro-4-piperidin-4-yl-phenylamino)-piperidine-2,6-dione hydrochloride

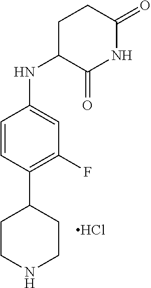 [SMILES: O=C1CCC(Nc2ccc(C3CCNCC3)c(F)c2)C(=O)N1 ; Cl]

Dioxane-HCl (4M, 30 mL, 130 mmol) was added to tert-butyl 4-[4-[(2,6-dioxo-3-piperidyl)amino]-2-fluoro-phenyl]piperidine-1-carboxylate (1.3 g, 3.21 mmol) at 10° C. the resulting mixture was warmed to ambient temperature and stirred for 16 h. The reaction mixture was concentrated under reduced pressure, triturated with ether and lyophilized to yield 3-[3-fluoro-4-(4-piperidyl)anilino]piperidine-2,6-dione (840 mg, 2.73 mmol, 85.25% yield) as green solid. LC MS: ES+ 306 (M+H⁺). ¹H NMR (400 MHz, DMSO-d₆) δ 10.79 (s, 1H), 9.00 (br s, 1H), 8.85-8.83 (m, 1H), 6.96-6.91 (m, 1H), 6.50-6.45 (m, 2H), 4.34-4.30 (m, 1H), 3.32-3.29 (m, 2H), 2.98-2.93 (m, 3H), 2.77-2.69 (m, 1H), 2.60-2.56 (m, 1H), 2.08-2.05 (m, 1H), 1.92-1.81 (m, 5H).

### Intermediate Synthesis of 3-(2-Fluoro-4-piperidin-4-yl-phenylamino)-piperidine-2,6-dione hydrochloride

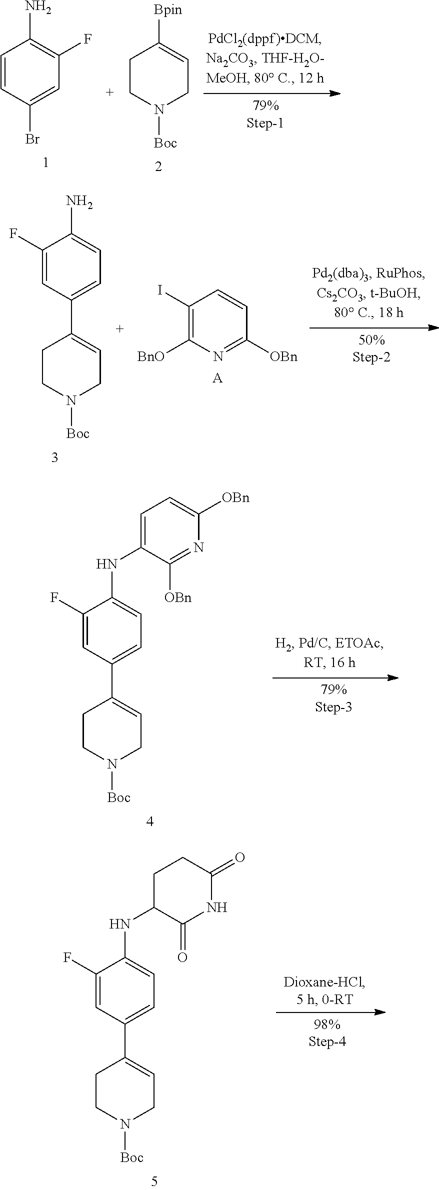

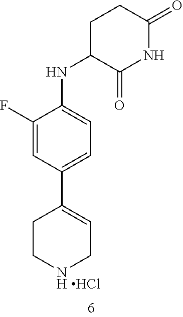

### Step-1: Preparation of 4-(4-Amino-3-fluoro-phenyl)-3, 6-dihydro-2H-pyridine-1-carboxylic acid tert-butyl ester

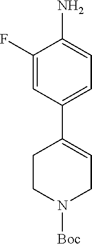 [SMILES: CC(C)(C)OC(=O)N1CC=C(c2ccc(N)c(F)c2)CC1]

Sodium carbonate (6.14 g, 57.89 mmol) was added to a stirred solution of 4-bromo-2-fluoro-aniline (5.00 g, 26.3 mmol) and tert-butyl 4-(4,4,5,5-tetramethyl-1,3,2-dioxaborolan-2-yl)-3,6-dihydro-2H-pyridine-1-carboxylate (8.95 g, 29.0 mmol) in water (12 mL), THF (60 mL) and methanol (24 mL). The resulting mixture was degassed with argon and PdCl₂(dppf)·dichloromethane (430 mg, 526 μmol) was added under inert atmosphere. The resulting mixture was heated at 80° C. for 12 h. The reaction mixture was diluted with ethyl acetate, filtered through a short pad of celite and washed with ethyl acetate. The combined organic extracts were washed with water, brine, dried over anhydrous sodium sulphate, filtered and concentrated under reduced pressure. The residue was purified by column chromatography (15% ethyl acetate-hexane) to yield tert-butyl 4-(4-amino-3-fluoro-phenyl)-3,6-dihydro-2H-pyridine-1-carboxylate (6.1 g, 20.9 mmol, 79% yield) as pale yellow solid. LC MS: ES+ 293 (M+H⁺).

### Step-2: Preparation of 4-[4-(2,6-Bis-benzyloxy-pyridin-3-ylamino)-3-fluoro-phenyl]-3,6-dihydro-2H-pyridine-1-carboxylic acid tert-butyl ester

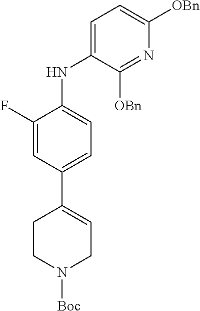 [SMILES: CC(C)(C)OC(=O)N1CC=C(c2ccc(Nc3ccc(OCc4ccccc4)nc3OCc3ccccc3)c(F)c2)CC1]

Cesium carbonate (19.73 g, 60.54 mmol) was added to a stirred solution of tert-butyl 4-(4-amino-3-fluoro-phenyl)-3,6-dihydro-2H-pyridine-1-carboxylate (5.9 g, 20.2 mmol) and 2,6-dibenzyloxy-3-iodo-pyridine (9.26 g, 22.2 mmol) in t-BuOH (60 mL). The resulting mixture was degassed with argon and Pd₂(dba)₃ (924 mg, 1.01 mmol) and RuPhos (942 mg, 2.02 mmol) were added under inert atmosphere. The resulting mixture was heated at 100° C. for 18 h. The reaction mixture was diluted with ethyl acetate, filtered through a short pad of celite and washed with ethyl acetate. The combined organic extracts were washed with water, brine, dried over anhydrous sodium sulphate, filtered and concentrated under reduced pressure. The residue was purified by column chromatography (10% ethyl acetate-hexane) to yield tert-butyl 4-[4-[(2,6-dibenzyloxy-3-pyridyl)amino]-3-fluoro-phenyl]-3,6-dihydro-2H-pyridine-1-carboxylate (5.9 g, 10.1 mmol, 50% yield) as pale yellow solid. LC MS: ES+ 582 (M+H⁺).

### Step-3: Preparation of 4-[4-(2,6-Dioxo-piperidin-3-ylamino)-3-fluoro-phenyl]-piperidine-1-carboxylic acid tert-butyl ester

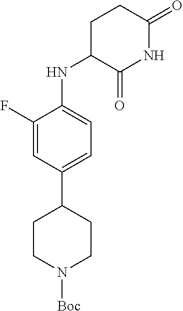 [SMILES: CC(C)(C)OC(=O)N1CCC(c2ccc(NC3CCC(=O)NC3=O)c(F)c2)CC1]

10% Pd—C (50% wet, 4.6 g) was added to a stirred degassed solution of tert-butyl 4-[4-[(2,6-dibenzyloxy-3-pyridyl)amino]-3-fluoro-phenyl]-3,6-dihydro-2H-pyridine-1-carboxylate (4.6 g, 7.91 mmol) in ethyl acetate (40 mL). The resulting mixture was stirred at ambient temperature under hydrogen balloon pressure for 20 h. The reaction mixture was filtered through a short pad of celite and washed with ethyl acetate. The combined filtrate was evaporated under reduced pressure and purified by column chromatography (40% ethyl acetate-hexane) to yield tert-butyl 4-[4-[(2,6-dioxo-3-piperidyl)amino]-3-fluoro-phenyl]piperidine-1-carboxylate (2.6 g, 6.41 mmol, 81% yield) as a blue solid. LC MS: ES+ 406 (M+H⁺).

### Step-4: Preparation of 3-(2-Fluoro-4-piperidin-4-yl-phenylamino)piperidine-2,6-dione hydrochloride

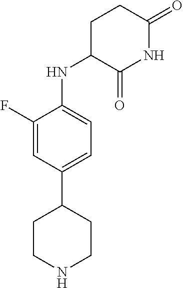 [SMILES: O=C1CCC(Nc2ccc(C3CCNCC3)cc2F)C(=O)N1]

Dioxane HCl (4M, 10 mL, 40 mmol) was added to tert-butyl 4-[4-[(2,6-dioxo-3-piperidyl)amino]-3-fluoro-phenyl]piperidine-1-carboxylate (1.3 g, 3.21 mmol) at 10° C. The resulting mixture was warmed to ambient temperature and stirred for 16 h. The reaction mixture was concentrated under reduced pressure, triturated with ether and lyophilized to yield 3-[2-fluoro-4-(4-piperidyl)anilino]piperidine-2,6-dione hydrochloride (840 mg, 2.73 mmol, 85% yield) as a green solid. LC MS: ES+ 306 (M+H⁺). ¹H NMR (400 MHz, DMSO-d₆) δ 10.82 (s, 1H), 8.85 (br s, 1H), 8.69-8.68 (m, 1H), 6.92-6.89 (m, 1H), 6.83-6.77 (m, 2H), 4.40-4.36 (m, 2H), 3.37-3.31 (m, 2H), 2.98-2.90 (m, 2H), 2.76-2.71 (m, 2H), 2.58-2.56 (m, 1H), 2.05-1.73 (m, 6H).

### Synthesis of (S)-4-(4-((2,6-dioxopiperidin-3-yl)amino)-2-fluorophenyl)piperidine hydrochloride and (R)-4-(4-((2,6-dioxopiperidin-3-yl)amino)-2-fluorophenyl)piperidine hydrochloride

### Step 1: Chiral separation to afford tert-butyl 4-(4-(((3S)-2,6-dioxopiperidin-3-yl)amino)-2-fluorophenyl)piperidine-1-carboxylate and tert-butyl 4-(4-(((3R)-2,6-dioxopiperidin-3-yl)amino)-2-fluorophenyl)piperidine-1-carboxylate

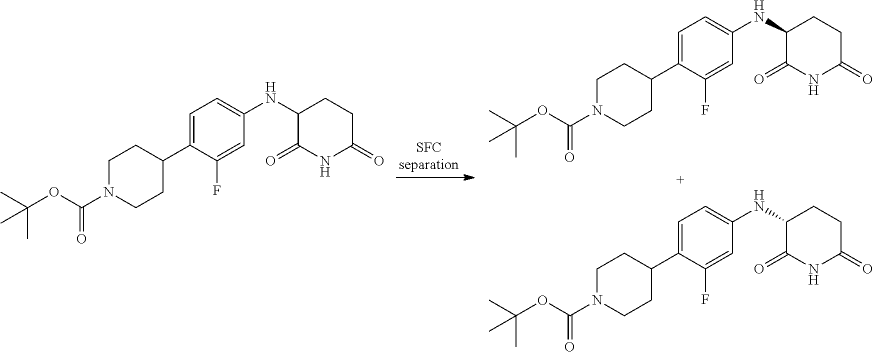 [SMILES: CC(C)(C)OC(=O)N1CCC(c2ccc(NC3CCC(=O)NC3=O)cc2F)CC1 ; CC(C)(C)OC(=O)N1CCC(c2ccc(N[C@@H]3CCC(=O)NC3=O)cc2F)CC1 ; CC(C)(C)OC(=O)N1CCC(c2ccc(N[C@H]3CCC(=O)NC3=O)cc2F)CC1]

Separation of racemic tert-butyl 4-(4-((2,6-dioxopiperidin-3-yl)amino)-2-fluorophenyl)piperidine-1-carboxylate (5.96 g) by chiral SFC was performed using the following method.
Column: ChiralCel OJ-H (250×21 mm), 5 um silica
Flow: 70 mL/min
Mobile Phase: 65% CO2+35% Isopropyl alcohol
ABPR: 100 bar
Temperature: 35° C.

The SFC separation afforded two sets of fractions. The earlier eluting fractions were lyophilized to afford tert-butyl 4-(4-(((3S)-2,6-dioxopiperidin-3-yl)amino)-2-fluorophenyl)piperidine-1-carboxylate (2.51 g, 42% yield, >99% enantiomeric excess, Chiral SFC Retention time=0.91 min).

The later eluting fractions were lyophilized to afford tert-butyl 4-(4-(((3R)-2,6-dioxopiperidin-3-yl)amino)-2-fluorophenyl)piperidine-1-carboxylate (2.69 g, 45% yield, 99.6% enantiomeric excess, Chiral SFC Retention time=1.26 min).

The retention time and enantiomeric excess of the two isolated isomers were determined by analytical chiral SFC, using the following conditions:
Column: ChiralCel OJ-H (100×4.6 mm)
Flow rate: 4 mL/min
Pressure: 100 bar
Temperature: 40° C.

### Step 2: Synthesis of (S)-4-(4-((2,6-dioxopiperidin-3-yl)amino)-2-fluorophenyl)piperidine hydrochloride

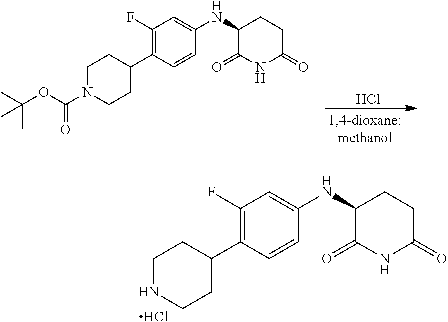 [SMILES: CC(C)(C)OC(=O)N1CCC(c2ccc(N[C@H]3CCC(=O)NC3=O)cc2F)CC1 ; Cl ; O=C1CC[C@H](Nc2ccc(C3CCNCC3)c(F)c2)C(=O)N1 ; Cl]

(S)-4-(4-((2,6-dioxopiperidin-3-yl)amino)-2-fluorophenyl)piperidine hydrochloride was obtained in quantitative yield from tert-butyl 4-(4-(((3S)-2,6-dioxopiperidin-3-yl)amino)-2-fluorophenyl)piperidine-1-carboxylate using the General procedure B. LCMS (ESI+): 306.3 [M+H⁺]

### Step 3: Synthesis of (R)-4-(4-((2,6-dioxopiperidin-3-yl)amino)-2-fluorophenyl)piperidine hydrochloride

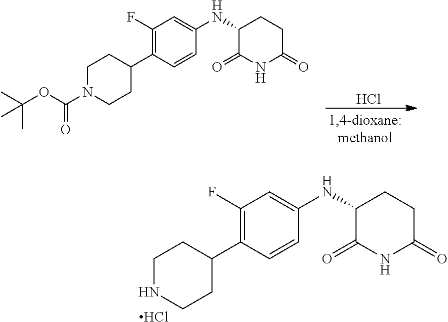 [SMILES: CC(C)(C)OC(=O)N1CCC(c2ccc(N[C@@H]3CCC(=O)NC3=O)cc2F)CC1 ; Cl ; O=C1CC[C@@H](Nc2ccc(C3CCNCC3)c(F)c2)C(=O)N1 ; Cl]

(R)-4-(4-((2,6-dioxopiperidin-3-yl)amino)-2-fluorophenyl)piperidine hydrochloride was obtained in quantitative yield from tert-butyl 4-(4-(((3S)-2,6-dioxopiperidin-3-yl)amino)-2-fluorophenyl)piperidine-1-carboxylate using the General procedure B. LCMS (ESI+): 306.3 (M+H⁺)

### Intermediate: 2-[4-[(2,6-dioxo-3-piperidyl)amino]phenyl]acetic acid

### Step-1: Preparation of tert-butyl 2-[4-[(2,6-dioxo-3-piperidyl)amino]phenyl]acetate

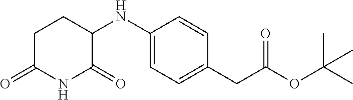 [SMILES: CC(C)(C)OC(=O)Cc1ccc(NC2CCC(=O)NC2=O)cc1]

3-Bromopiperidine-2,6-dione (13.9 g, 72.4 mmol), followed by sodium bicarbonate (12.2 g, 145 mmol) were added to a stirred solution of tert-butyl 2-(4-aminophenyl)acetate (10.0 g, 48.3 mmol) in DMF (80 mL) in a sealed tube. The resulting mixture was heated at 70° C. for 24 h. The reaction mixture was diluted with water and extracted with ethyl acetate. The combined organic extracts were washed with brine, dried over anhydrous sodium sulphate, filtered and evaporated under reduced pressure. The residue was purified by silica gel chromatography (35% ethyl acetate-hexane) to yield tert-butyl 2-[4-[(2,6-dioxo-3-piperidyl)amino]phenyl]acetate (7.5 g, 23.6 mmol, 49% yield). LC MS: ES+ 319 (M+H⁺)

### Step-2: Preparation of 2-[4-[(2,6-dioxo-3-piperidyl)amino]phenyl]acetic acid

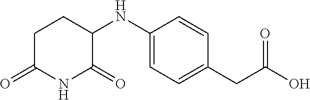 [SMILES: O=C(O)Cc1ccc(NC2CCC(=O)NC2=O)cc1]

TFA (8.47 mL, 110 mmol) was added drop-wise at 0° C. to a stirred solution of tert-butyl 2-[4-[(2,6-dioxo-3-piperidyl)amino]phenyl]acetate (3.5 g, 10.99 mmol) in dichloromethane (45 mL. The resulting mixture was warmed to ambient temperature and stirred for 5 h. The reaction mixture was concentrated under reduced pressure, triturated with MTBE and lyophilized to yield 2-[4-[(2,6-dioxo-3-piperidyl)amino]phenyl]acetic acid (2.9 g, 10.9 mmol, 99% yield) as a grey solid. LC MS: ES+ 263 (M+H⁺). ¹H NMR (400 MHz, DMSO-d₆) δ 10.79 (s, 1H), 6.97 (d, J=8.36 Hz, 2H), 6.63 (d, J=8.36 Hz, 2H), 4.32-4.28 (m, 1H), 3.37 (s, 2H), 2.78-2.71 (m, 1H), 2.61-2.54 (m, 1H), 2.13-2.07 (m, 1H), 1.92-1.81 (m, 1H)

### Intermediate: Synthesis of 1-(6-bromo-1-methyl-indazol-3-yl)hexahydropyrimidine-2,4-dione

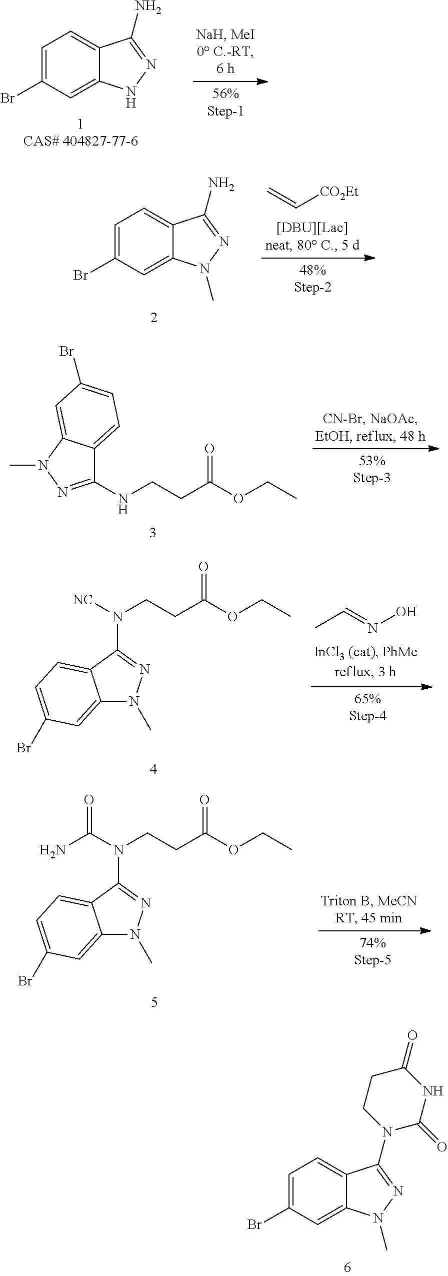

### Step-1: Preparation of 6-bromo-1-methyl-indazol-3-amine

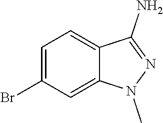

Sodium hydride (60% in oil 2.38 g, 59.4 mmol) was added portion wise at 0° C. to a stirred solution of 6-bromo-1H-indazol-3-amine (7 g, 33.0 mmol, 439 μL) in DMF (150 mL) and the mixture was stirred for 40 min. Iodomethane (5.15 g, 36.3 mmol, 2.26 mL) was added drop-wise under cooling and the resulting mixture was warmed to ambient temperature and stirred for 16 h. The reaction mixture was quenched with saturated ammonium chloride solution and extracted with ethyl acetate. The combined organic extracts were washed with water, brine, dried over anhydrous sodium sulphate, filtered and concentrated under reduced pressure. The residue was purified by silica gel chromatography (50% ethyl acetate-hexane) to yield 6-bromo-1-methyl-indazol-3-amine (4.2 g, 18.6 mmol, 56% yield). LC MS: ES+ 227 (M+H⁺)

### Step-2: Preparation of ethyl 3-[(6-bromo-1-methyl-indazol-3-yl)amino]propanoate

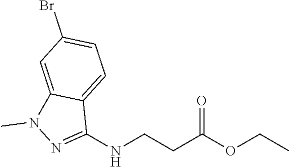 [SMILES: CCOC(=O)CCNc1nn(C)c2cc(Br)ccc12]

Ethyl acrylate (14.0 g, 139 mmol) was added in 5 portions (2.8 g each) over 5 days to a mixture of 6-bromo-1-methyl-indazol-3-amine (4.2 g, 18.6 mmol), [DBU][Lac] (prepared by mixing equimolar mixture of DBU and lactic acid with stirring for 16 h at ambient temperature, 2.09 g, 14.9 mmol) at 80° C. After completion (LCMS), the reaction mixture was quenched with sodium hypochlorite (30% aq, 5 mL) and diluted with ethyl acetate. The combined organics were washed with water, brine, dried over anhydrous sodium sulphate, filtered and concentrated under reduced pressure. The residue was purified by silica gel chromatography (50% ethyl acetate-hexane) to yield ethyl 3-[(6-bromo-1-methyl-indazol-3-yl)amino]propanoate (2.9 g, 8.89 mmol, 48% yield). LCMS (ESI+): 327 (M+H⁺).

### Step-3: Preparation of ethyl 3-[(6-bromo-1-methyl-indazol-3-yl)-cyano-amino]propanoate

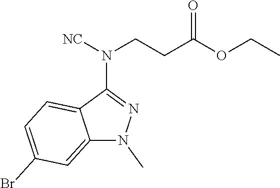 [SMILES: CCOC(=O)CCN(C#N)c1nn(C)c2cc(Br)ccc12]

Anhydrous sodium acetate (1.46 g, 17.8 mmol), followed by cyanogen bromide (1.41 g, 13.3 mmol) were added to a stirred solution of ethyl 3-[(6-bromo-1-methyl-indazol-3-yl)amino]propanoate (2.9 g, 8.89 mmol) in ethanol (40 mL) at ambient temperature. The resulting mixture was heated to reflux for 48 h. The reaction mixture was concentrated under reduced pressure and diluted with ethyl acetate. The combined organics were washed with water, brine, dried over anhydrous sodium sulphate, filtered and concentrated under reduced pressure. The residue was purified by column chromatography (45% ethyl acetate-hexane) to yield ethyl 3-[(6-bromo-1-methyl-indazol-3-yl)-cyano-amino]propanoate (1.65 g, 4.70 mmol, 53% yield). LC MS: ES+ 352 (M+H⁺).

### Step-4: Preparation of ethyl 3-[(6-bromo-1-methyl-indazol-3-yl)-carbamoyl-amino]propanoate

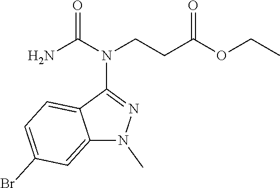 [SMILES: CCOC(=O)CCN(C(N)=O)c1nn(C)c2cc(Br)ccc12]

(1E)-Acetaldehyde oxime (1.01 g, 17.1 mmol), followed by indium (III) chloride (126 mg, 569 μmol) were added to a stirred solution of ethyl 3-[(6-bromo-1-methyl-indazol-3-yl)-cyano-amino]propanoate (2 g, 5.69 mmol) in toluene (60 mL) at ambient temperature. The resulting mixture was heated to reflux for 1 h. The reaction mixture was diluted with ethyl acetate, washed with water and brine. The organics were dried over anhydrous sodium sulphate, filtered and concentrated under reduced pressure. The residue was purified by column chromatography (60% ethyl acetate-hexane) to yield ethyl 3-[(6-bromo-1-methyl-indazol-3-yl)-carbamoyl-amino]propanoate (1.4 g, 3.79 mmol, 67% yield). LC MS: ES+ 370 (M+H⁺).

### Step-5: Preparation of 1-(6-bromo-1-methyl-indazol-3-yl)hexahydropyrimidine-2,4-dione

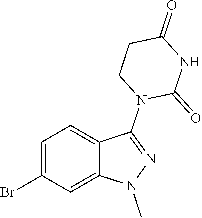 [SMILES: Cn1nc(N2CCC(=O)NC2=O)c2ccc(Br)cc21]

Triton-B (40% in methanol, 2.4 mL, 5.69 mmol) was added drop-wise to a stirred solution of ethyl 3-[(6-bromo-1-methyl-indazol-3-yl)-carbamoyl-amino]propanoate (1.40 g, 3.79 mmol) in MeCN (70 mL) at ambient temperature. The resulting mixture was stirred at ambient temperature for 45 minutes. The reaction mixture was concentrated under vacuum and diluted with ethyl acetate. The organic layer was washed with water, brine, dried over anhydrous sodium sulphate, filtered and concentrated under reduced pressure. The residue was purified by column chromatography (30% ethyl acetate-hexane) to yield 1-(6-bromo-1-methyl-indazol-3-yl)hexahydropyrimidine-2,4-dione (910 mg, 2.81 mmol, 74% yield) as white solid. LC MS: ES+ 324 (M+H⁺). ¹H NMR (400 MHz, DMSO-d₆) δ 10.60 (s, 1H), 7.97 (s, 1H), 7.61 (d, J=8.6 Hz, 1H), 7.26-7.23 (m, 1H), 3.98 (s, 3H), 3.93 (t, J=6.6 Hz, 2H), 2.76 (t, J=6.6 Hz, 2H).

### Preparation of 1-(1-methyl-6-(piperidin-4-yl)-1H-indazol-3-yl)dihydropyrimidine-2,4(1H,3H)-dione hydrochloride

### Step 1: tert-butyl 4-[3-(2,4-dioxohexahydropyrimidin-1-yl)-1-methyl-indazol-6-yl]-3,6-dihydro-2H-pyridine-1-carboxylate

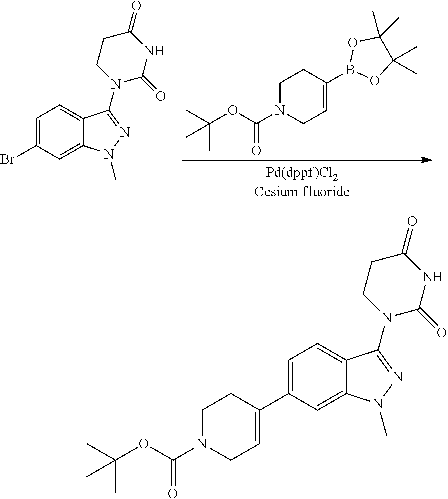

A solution of 1-(6-bromo-1-methyl-indazol-3-yl)hexahydropyrimidine-2,4-dione (1.25 g, 3.87 mmol) and tert-butyl 4-(4,4,5,5-tetramethyl-1,3,2-dioxaborolan-2-yl)-3,6-dihydro-2H-pyridine-1-carboxylate (2.39 g, 7.74 mmol) was bubbled with N₂ for 10 min. Then, cesium fluoride (1.18 g, 7.74 mmol) and Pd(dppf)Cl₂ (566 mg, 774 μmol) were added and the mixture was stirred at 85° C. for 2 h. The mixture was cooled to ambient temperature, diluted with ethyl acetate and filtered through Celite/silica gel. After washing with ethyl acetate, the filtrate was diluted with water and layers were separated, and the organic layer was washed with brine, dried over Na₂SO₄, filtered, and concentrated. The residue was purified by normal phase chromatography (5-100% ethyl acetate in Hexanes) to afford tert-butyl 4-[3-(2,4-dioxohexahydropyrimidin-1-yl)-1-methyl-indazol-6-yl]-3,6-dihydro-2H-pyridine-1-carboxylate (1.04 g, 2.44 mmol, 63% yield). LCMS (ESI+): 426.3 (M+H⁺)

### Step 2: tert-butyl 4-[3-(2,4-dioxohexahydropyrimidin-1-yl)-1-methyl-indazol-6-yl]piperidine-1-carboxylate

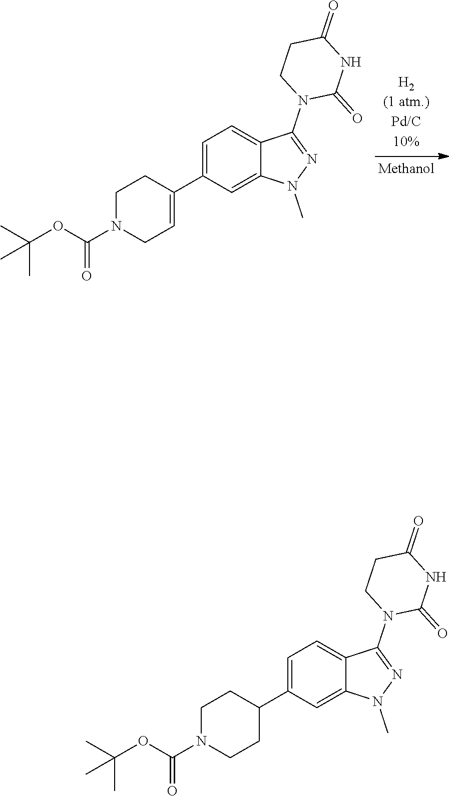 [SMILES: Cn1nc(N2CCC(=O)NC2=O)c2ccc(C3=CCN(C(=O)OC(C)(C)C)CC3)cc21 ; Cn1nc(N2CCC(=O)NC2=O)c2ccc(C3CCN(C(=O)OC(C)(C)C)CC3)cc21]

Palladium (10% on carbon, Type 487, dry, 1.08 g, 1.02 mmol) was added to a solution of tert-butyl 4-[3-(2,4-dioxohexahydropyrimidin-1-yl)-1-methyl-indazol-6-yl]-3,6-dihydro-2H-pyridine-1-carboxylate (1.44 g, 3.38 mmol) in methanol (30 mL) and the mixture was stirred at ambient temperature under a hydrogen balloon atmosphere. After 24 h, the reaction mixture was filtered through a pad of celite, washed with a mixture of dichloromethane/methanol (1:1), and concentrated in vacuo to yield tert-butyl 4-[3-(2,4-dioxohexahydropyrimidin-1-yl)-1-methyl-indazol-6-yl]piperidine-1-carboxylate (1.42 g, 3.32 mmol, 98% yield). LCMS (ESI+): 372.3 (M-tert-butyl+H⁺).

### Step 3: 1-(1-methyl-6-(piperidin-4-yl)-1H-indazol-3-yl)dihydropyrimidine-2,4(1H,3H)-dione hydrochloride

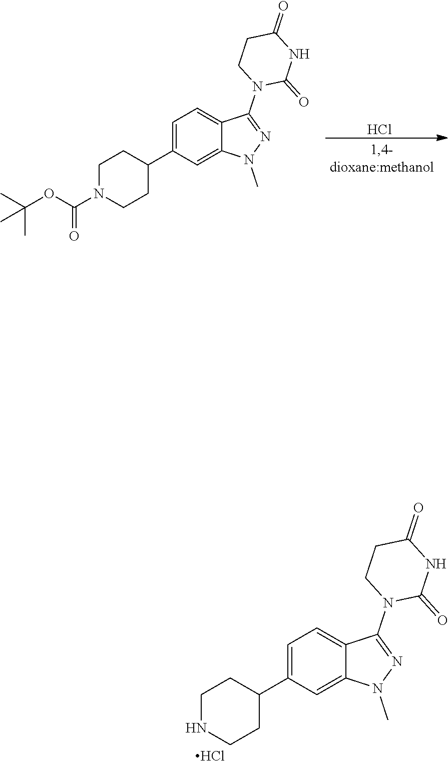 [SMILES: Cn1nc(N2CCC(=O)NC2=O)c2ccc(C3CCN(C(=O)OC(C)(C)C)CC3)cc21 ; Cl ; Cn1nc(N2CCC(=O)NC2=O)c2ccc(C3CCNCC3)cc21 ; Cl]

1-(1-methyl-6-(piperidin-4-yl)-1H-indazol-3-yl)dihydropyrimidine-2,4(1H,3H)-dione hydrochloride was obtained in quantitative yield from tert-butyl 4-[3-(2,4-dioxohexahydropyrimidin-1-yl)-1-methyl-indazol-6-yl]-3,6-dihydro-2H-pyridine-1-carboxylate using the general method B for tert-butoxycarbonyl protecting group deprotection. LCMS (ESI+): 328.1 (M+H⁺).

### Synthesis of 1-((4-(piperidin-4-yl)phenyl)amino)-3-azabicyclo[3.1.1]heptane-2,4-dione hydrochloride

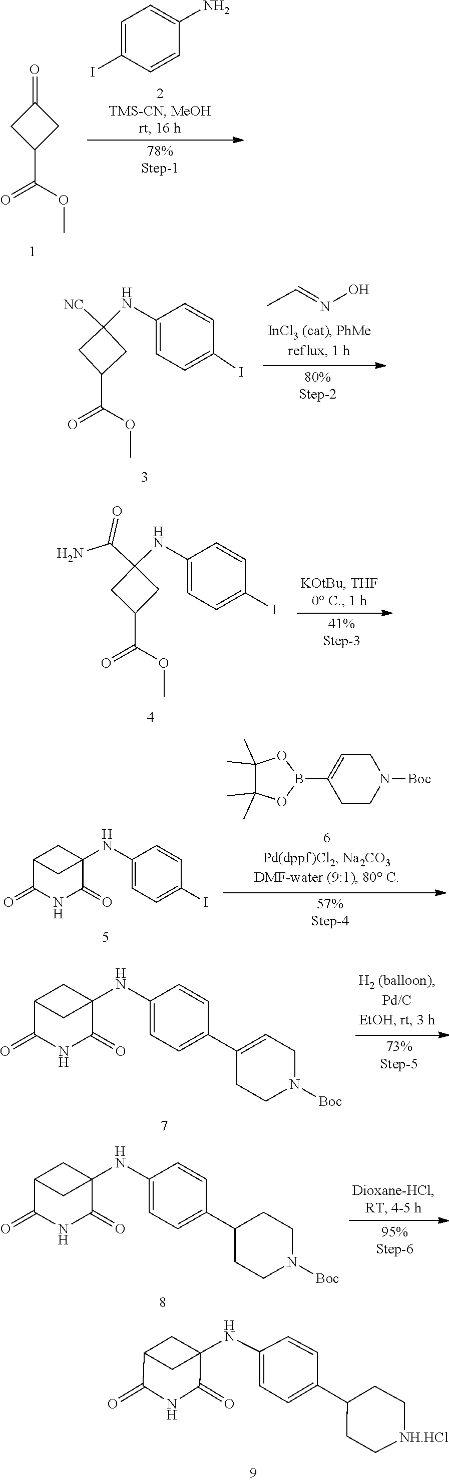

### Step-1: Preparation of 3-Cyano-3-(4-iodo-phenylamino)-cyclobutane carboxylic acid methyl ester

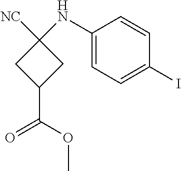 [SMILES: COC(=O)C1CC(C#N)(Nc2ccc(I)cc2)C1]

4-Iodoaniline (13.2 g, 60.1 mmol) followed by trimethylsilyl cyanide (10.8 g, 109 mmol, 13.7 mL) were added to a stirred solution of methyl 3-oxocyclobutanecarboxylate (7 g, 54.6 mmol) in methanol (270 mL). The resulting mixture was stirred at ambient temperature for 16 h. The reaction mixture was concentrated under reduced pressure and the residue was purified by silica gel chromatography (5-10% ethyl acetate-hexane) to afford methyl 3-cyano-3-(4-iodoanilino)cyclobutanecarboxylate (15.2 g, 42.7 mmol, 78% yield) as an off-white solid. LCMS ES+ 357 (M+H⁺)

### Step-2: Preparation of 3-Carbamoyl-3-(4-iodo-phenylamino)-cyclobutane carboxylic acid methyl ester

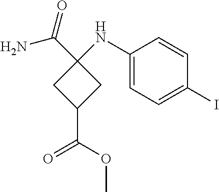 [SMILES: COC(=O)C1CC(Nc2ccc(I)cc2)(C(N)=O)C1]

Acetaldehyde oxime (4.98 g, 84.2 mmol), followed by indium chloride (62.1 mg, 281 μmol) were added to a stirred solution of methyl 3-cyano-3-(4-iodoanilino) cyclobutanecarboxylate (10 g, 28.1 mmol) in toluene (120 mL) at ambient temperature. The resulting mixture was heated to reflux for 1 h. After completion, the reaction mixture was cooled to ambient temperature and the precipitate thus formed was filtered, washed with toluene:ether (1:1) and dried to yield methyl 3-carbamoyl-3-(4-iodoanilino) cyclobutanecarboxylate (8.4 g, 22.5 mmol, 80% yield). It was used in the next step without further purification. LCMS (ESI+): 375 (M+H⁺)

### Step-3: Preparation of 1-(4-Iodo-phenylamino)-3-aza-bicyclo[3.1.1]heptane-2,4-dione

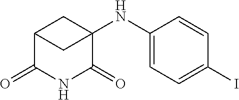 [SMILES: O=C1NC(=O)C2(Nc3ccc(I)cc3)CC1C2]

Potassium tert-butoxide (4.62 g, 41.2 mmol) was added at 0° C. to a stirred solution of methyl 3-[2-amino-1-(4-iodoanilino)-2-oxo-ethyl]cyclobutanecarboxylate (8 g, 20.6 mmol) in THF (150 mL), and the reaction mixture was stirred for 1 h at 0° C. The reaction mixture was neutralized with 1M citric acid solution and adjusted to pH-6 and extracted with ethyl acetate. The combined organic layers were dried over sodium sulfate, filtered and concentrated under reduced pressure. The residue mass was purified by column chromatography (40% ethyl acetate/hexane) to afford 5-(4-iodoanilino)-3-azabicyclo[3.1.1]heptane-2,4-dione (2.9 g, 8.48 mmol, 41% yield). LCMS (ESI+): 343 (M+H⁺)

### Step-4: Preparation of 4-[4-(2,4-Dioxo-3-aza-bicyclo[3.1.1]hept-1-ylamino)-phenyl]-3,6-dihydro-2H-pyridine1-carboxylic acid tert-butyl ester

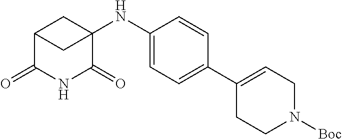 [SMILES: CC(C)(C)OC(=O)N1CC=C(c2ccc(NC34CC(C3)C(=O)NC4=O)cc2)CC1]

Sodium carbonate (1.98 g, 18.7 mmol) was added to a stirred solution of 5-(4-iodoanilino)-3-azabicyclo[3.1.1]heptane-2,4-dione (2.9 g, 8.48 mmol) and tert-butyl 4-(4,4,5,5-tetramethyl-1,3,2-dioxaborolan-2-yl)-3,6-dihydro-2H-pyridine-1-carboxylate (5.24 g, 17.0 mmol) in DMF (32 mL) and water (8 mL) and the reaction was degassed with argon. Pd(dppf)Cl₂ (692 mg, 848 μmol) was added under inert atmosphere. The resulting mixture was heated at 80° C. for 16 h. The reaction mixture was diluted with ethyl acetate and filtered through a short pad of celite. The filtrate was washed with water, brine, dried over anhydrous sodium sulphate, filtered and concentrated under reduced pressure. The residue was purified by column chromatography (5-10% ethyl acetate-hexane) to yield tert-butyl 4-[4-[(2,4-dioxo-3-azabicyclo[3.1.1]heptan-5-yl)amino]phenyl]-3,6-dihydro-2H-pyridine-1-carboxylate (1.91 g, 4.81 mmol, 57% yield). LCMS ES+ 398 (M+H⁺)

### Step-5: Preparation of 4-[4-(2,4-Dioxo-3-aza-bicyclo[3.1.1]hept-1-ylamino)-phenyl]-piperidine-1-carboxylic acid tert-butyl ester

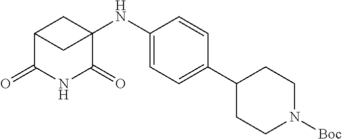 [SMILES: CC(C)(C)OC(=O)N1CCC(c2ccc(NC34CC(C3)C(=O)NC4=O)cc2)CC1]

10% Pd—C (50% wet, 1 g) was added to a degassed solution of tert-butyl 4-[4-[(2,4-dioxo-3-azabicyclo[3.1.1]heptan-5-yl)amino]phenyl]-3,6-dihydro-2H-pyridine-1-carboxylate (1.91 g, 4.81 mmol) in ethanol (20 mL). The resulting mixture was stirred at ambient temperature under a hydrogen balloon atmosphere for 3 h. After completion (confirmed by LCMS), the reaction mixture was filtered through a short pad of celite, washed with ethyl acetate and concentrated under reduced pressure. The residue was purified by silica gel chromatography (60-70% ethyl acetate-hexane) to yield tert-butyl 4-[4-[(2,4-dioxo-3-azabicyclo[3.1.1]heptan-5-yl)amino]phenyl]piperidine-1-carboxylate (1.4 g, 3.50 mmol, 73% yield) LCMS ES+ 400 (M+H⁺)

### Step-6: Preparation of 5-(4-Piperidin-4-yl-phenylamino)-3-aza-bicyclo[3.1.1]heptane-2,4-dione hydrochloride

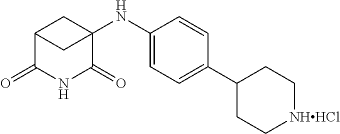

Dioxane HCl (4M, 15 mL, 60 mmol) was added to tert-butyl 4-[4-[(2,4-dioxo-3-azabicyclo[3.1.1]heptan-5-yl)amino]phenyl]piperidine-1-carboxylate (1.4 g, 3.50 mmol) at 10° C. The resulting mixture was warmed to ambient temperature and stirred for 5 h. The reaction mixture was concentrated under reduced pressure, triturated with ether and lyophilized to yield 5-[4-(4-piperidyl)anilino]-3-azabicyclo[3.1.1]heptane-2,4-dione hydrochloride (1.08 g, 3.34 mmol, 95% yield) as an off white solid. LCMS ES+ 300 (M+H⁺), ¹H-NMR (400 MHz, DMSO-d₆) δ 10.72 (s, 1H), 8.95 (br s, 1H), 8.81-8.79 (m, 1H), 6.90 (d, J=8.2 Hz, 2H), 6.44 (d, J=8.16 Hz, 2H), 3.32-3.29 (m, 2H), 2.95-2.91 (m, 3H), 2.73-2.62 (m, 3H), 2.49 (br m, 2H), 1.85-1.72 (m, 4H).

### Synthesis of 3-[3-(difluoromethyl)-4-(4-piperidyl)anilino]piperidine-2,6-dione hydrochloride

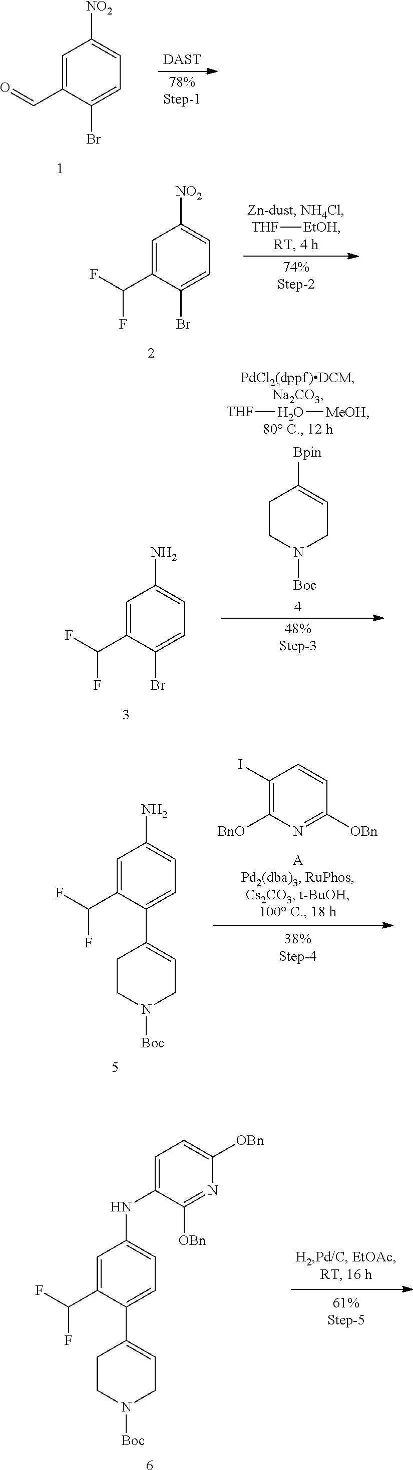

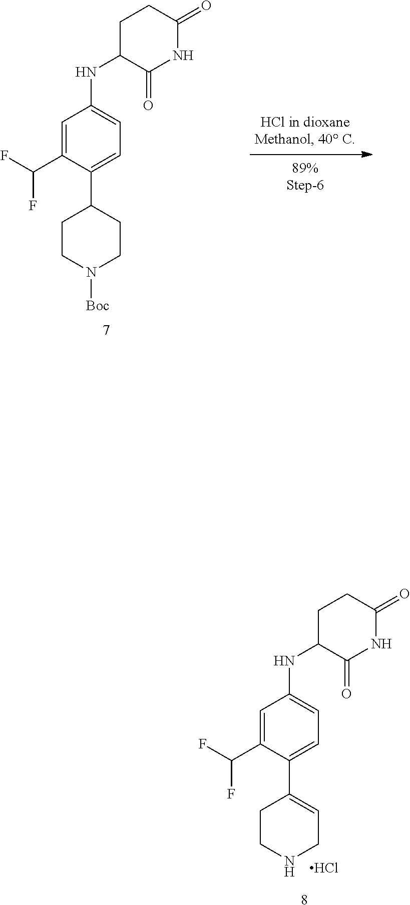

### Step-1: Synthesis of 1-Bromo-2-difluoromethyl-4-nitro-benzene

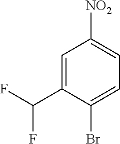 [SMILES: O=[N+]([O-])c1ccc(Br)c(C(F)F)c1]

DAST (24.13 mL, 182.60 mmol) was added to a stirred solution of 2-bromo-5-nitro-benzaldehyde (7 g, 30.4 mmol) in dichloromethane (350 mL) at 0° C. and the resulting reaction mixture was stirred at ambient temperature for 16 h. After completion, the reaction mixture was basified with 10% NaHCO₃ solution and extracted with dichloromethane. The combined organic extracts were washed with water, brine, dried over sodium sulphate, filtered and concentrated under reduced pressure. The residue was purified by column chromatography (10% ethyl acetate/hexane) to afford 1-bromo-2-(difluoromethyl)-4-nitro-benzene (6 g, 23.8 mmol, 78% yield).

### Step-2: Synthesis of 4-Bromo-3-difluoromethyl-phenylamine

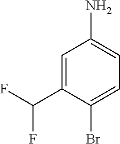 [SMILES: Nc1ccc(Br)c(C(F)F)c1]

Ammonium chloride (12.7 g, 238 mmol) and zinc (15.6 g, 238 mmol) were added to a stirred solution of 1-bromo-2-(difluoromethyl)-4-nitro-benzene (6.0 g, 23.8 mmol) in THF (70 mL) and ethanol (70 mL) at ambient temperature. The resulting mixture was stirred at ambient temperature for 4 h. After completion, reaction mixture was filtered through a short pad of celite and washed with ethanol. The filtrate was concentrated under reduced pressure and the residue purified by column chromatography (40% ethyl acetate-hexane) to afford 4-bromo-3-(difluoromethyl) aniline (3.95 g, 17.8 mmol, 75% yield). LC MS: ES+ 221 (M+H⁺).

### Step-3: Synthesis of 4-(4-Amino-2-difluoromethyl-phenyl)-3,6-dihydro-2H-pyridine-1-carboxylic acid tert-butyl ester

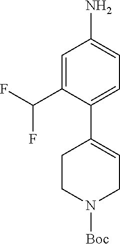 [SMILES: CC(C)(C)OC(=O)N1CC=C(c2ccc(N)cc2C(F)F)CC1]

Sodium carbonate (3.06 g, 28.82 mmol) was added to a stirred solution of 4-bromo-3-(difluoromethyl)aniline (3.2 g, 14.4 mmol) and tert-butyl 4-methyl-3,6-dihydro-2H-pyridine-1-carboxylate (3.08 g, 15.9 mmol) in THF (20 mL), methanol (10 mL) and water (10 mL) and the mixture was thoroughly purged with argon. PdCl₂(dppf)·dichloromethane (2.35 g, 2.88 mmol) was added under inert atmosphere. Resulting mixture was heated at 80° C. for 12 h. After completion, the reaction mixture was diluted with ethyl acetate, filtered through a short pad of celite and washed with ethyl acetate. The combined organic part was washed with water, brine, dried over anhydrous sodium sulphate, filtered and concentrated under reduced pressure. The residue was purified by column chromatography (20% ethyl acetate-hexane) to afford tert-butyl 4-[4-amino-2-(difluoromethyl)phenyl]-3,6-dihydro-2H-pyridine-1-carboxylate (2.24 g, 6.91 mmol, 48% yield). LC MS: ES+ 325 (M+H⁺).

### Step-4: Synthesis of 4-[4-(2,6-Bis-benzyloxy-pyridin-3-ylamino)-2-difluoromethyl-phenyl]-3,6-dihydro-2H pyridine-1-carboxylic acid tert-butyl ester

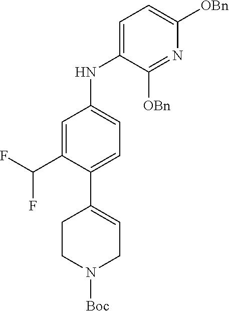 [SMILES: CC(C)(C)OC(=O)N1CC=C(c2ccc(Nc3ccc(OCc4ccccc4)nc3OCc3ccccc3)cc2C(F)F)CC1]

Cesium carbonate (5.12 g, 15.72 mmol) was added to a stirred solution of tert-butyl 4-[4-amino-2-(difluoromethyl)phenyl]-3,6-dihydro-2H-pyridine-1-carboxylate (1.7 g, 5.24 mmol) and 2,6-dibenzyloxy-3-iodo-pyridine (2.41 g, 5.77 mmol) in tert Butanol (40 mL). The resulting mixture was degassed with argon and Pd₂(dba)₃ (96 mg, 1.05 mmol), Ruphos (978 mg, 2.10 mmol) were added under inert atmosphere. The resulting mixture was heated at 100° C. for 18 h. After completion, the reaction mixture was diluted with ethyl acetate, filtered through a short pad of celite and washed with ethyl acetate. The filtrate was washed with water, brine, dried over anhydrous sodium sulphate, filtered and concentrated under reduced pressure. The residue was purified by column chromatography (25% ethyl acetate-hexane) to afford tert-butyl 4-[4-[(2,6-dibenzyloxy-3-pyridyl)amino]-2-(difluoromethyl)phenyl]-3,6-dihydro-2H-pyridine-1-carboxylate (1.23 g, 2.00 mmol, 38% yield). LC MS: ES+ 614 (M+H⁺).

### Step-5: Synthesis of 4-[2-Difluoromethyl-4-(2,6-dioxo-piperidin-3-ylamino)-phenyl]-piperidine-1-carboxylic acid tert-butyl ester

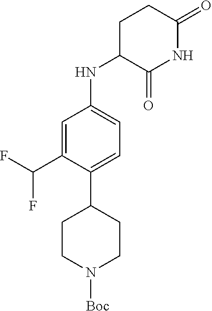 [SMILES: CC(C)(C)OC(=O)N1CCC(c2ccc(NC3CCC(=O)NC3=O)cc2C(F)F)CC1]

10% Pd—C (50% wet, 2 g) was added to a degassed solution of tert-butyl 4-[4-[(2,6-dibenzyloxy-3-pyridyl)amino]-2-(difluoromethyl)phenyl]-3,6-dihydro-2H-pyridine-1-carboxylate (2 g, 3.26 mmol) in ethyl acetate (15 mL). The resulting mixture was stirred at ambient temperature under a hydrogen balloon atmosphere for 16 h. After completion, the reaction mixture was filtered through a short pad of celite, washed with ethyl acetate and concentrated under reduced pressure. The residue was purified by silica gel chromatography (60% ethyl acetate in hexane) to afford tert-butyl 4-[2-(difluoromethyl)-4-[(2,6-dioxo-3-piperidyl)amino]phenyl]piperidine-1-carboxylate (880 mg, 1.99 mmol, 61% yield) as a light blue solid. LC MS: ES+ 438 (M+H⁺). ¹H NMR (400 MHz, DMSO-d₆) δ 10.77 (s, 1H), 7.26-6.98 (m, 2H), 6.81 (s, 1H), 6.77 (d, J=8.8 Hz, 1H), 6.03 (d, J=7.8 Hz, 1H), 4.34 (bs, 1H), 4.06-4.03 (m, 2H), 2.90-2.70 (m, 4H), 2.60-2.56 (m, 1H), 2.09-2.06 (m, 1H), 1.91-1.87 (m, 1H), 1.61-1.59 (m, 2H), 1.51-1.46 (m, 2H), 1.41 (s, 9H).

### Step 6: Synthesis of 3-[3-(difluoromethyl)-4-(4-piperidyl)anilino]piperidine-2,6-dione hydrochloride

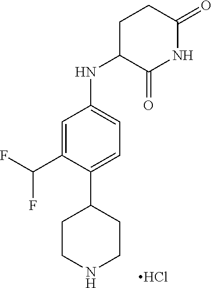 [SMILES: O=C1CCC(Nc2ccc(C3CCNCC3)c(C(F)F)c2)C(=O)N1 ; Cl]

tert-Butyl 4-[2-(difluoromethyl)-4-[(2,6-dioxo-3-piperidyl)amino]phenyl]piperidine-1-carboxylate (191 mg, 436.59 μmol) was dissolved in a methanol (3 mL) and hydrogen chloride solution (4.0M in dioxane, 1.09 mL) was added. The reaction mixture was heated at 40° C. for 4 h, and the reaction was complete. The volatiles were evaporated under reduce pressure. The material was submitted to high vacuum, frozen to −78° C. and thawed to afford 3-[3-(difluoromethyl)-4-(4-piperidyl)anilino]piperidine-2,6-dione hydrochloride (145 mg, 388 μmol, 89% yield) as a dense off-white solid. LCMS (ESI+): Rt=0.954 min., MS (ESI+): 338.3 (M+H⁺).

### Synthesis of 5-[(2,6-dioxo-3-piperidyl)amino]-2-(4-piperidyl)benzonitrile hydrochloride

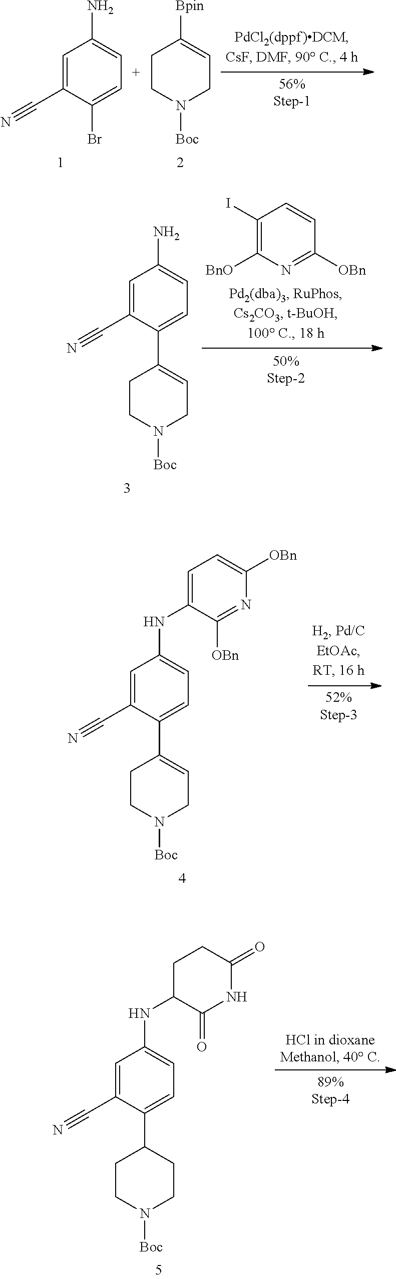

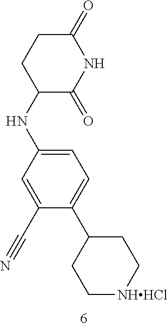

### Step-1: Synthesis of 4-(4-Amino-2-cyano-phenyl)-3,6-dihydro-2H-pyridine-1-carboxylic acid tert-butyl ester

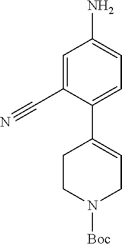 [SMILES: CC(C)(C)OC(=O)N1CC=C(c2ccc(N)cc2C#N)CC1]

To a stirred solution of 5-amino-2-bromo-benzonitrile (5 g, 25.38 mmol) and tert-butyl 4-(4,4,5,5-tetramethyl-1,3,2-dioxaborolan-2-yl)-3,6-dihydro-2H-pyridine-1-carboxylate (11.77 g, 38.06 mmol) in DMF (60 mL) was added cesium fluoride (7.71 g, 50.75 mmol, 1.87 mL) and the reaction mixture was degassed with argon. PdCl₂(dppf)·dichloromethane (4.14 g, 5.08 mmol) was added under inert atmosphere. Resulting mixture was heated at 90° C. for 16 h. After completion, reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (20% ethyl acetate-hexane) to afford tert-butyl 4-(4-amino-2-cyano-phenyl)-3,6-dihydro-2H-pyridine-1-carboxylate (4.3 g, 14.36 mmol, 56.60% yield). LC MS: ES+ 300 (M+H).

### Step-2: Synthesis of 4-[4-(2,6-Bis-benzyloxy-pyridin-3-ylamino)-2-cyano-phenyl]-3,6-dihydro-2H-pyridine-1-carboxylic acid tert-butyl ester

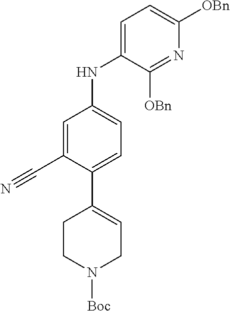 [SMILES: CC(C)(C)OC(=O)N1CC=C(c2ccc(Nc3ccc(OCc4ccccc4)nc3OCc3ccccc3)cc2C#N)CC1]

To a stirred solution of tert-butyl 4-(4-amino-2-cyano-phenyl)-3,6-dihydro-2H-pyridine-1-carboxylate (3 g, 10.02 mmol) and 2,6-dibenzyloxy-3-iodo-pyridine (4.60 g, 11.02 mmol) in t-BuOH (50 mL), cesium carbonate (9.80 g, 30.06 mmol) was added. Resulting mixture was degassed with argon and Pd₂(dba)₃ (458.83 mg, 501.06 μmol), RuPhos (467.62 mg, 1.00 mmol) were added under inert atmosphere. Resulting mixture was heated at 100° C. for 18 h. The reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (25% ethyl acetate-hexane) to afford tert-butyl 4-[2-cyano-4-[(2,6-dibenzyloxy-3-pyridyl)amino]phenyl]-3,6-dihydro-2H-pyridine-1-carboxylate (3 g, 5.10 mmol, 50.85% yield) LC MS: ES+ 589 (M+H).

### Step-3: Synthesis of 4-[2-Cyano-4-(2,6-dioxo-piperidin-3-ylamino)-phenyl]-piperidine-1-carboxylic acid tert-butyl ester

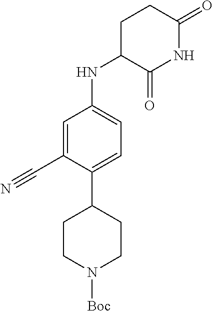 [SMILES: CC(C)(C)OC(=O)N1CCC(c2ccc(NC3CCC(=O)NC3=O)cc2C#N)CC1]

To a degassed solution of tert-butyl 4-[2-cyano-4-[(2,6-dibenzyloxy-3-pyridyl)amino]phenyl]-3,6-dihydro-2H-pyridine-1-carboxylate (3 g, 5.10 mmol) in ethyl acetate (60 mL), 10% Pd—C (50% wet, 3 g) was added. Resulting mixture was stirred at ambient temperature under hydrogen at balloon pressure for 16 h. The reaction mixture was filtered through a short pad of celite, washed with ethyl acetate and concentrated under reduced pressure. The crude mass was purified by silica gel column chromatography (60% ethyl acetate in hexane) to afford tert-butyl 4-[2-cyano-4-[(2,6-dioxo-3-piperidyl)amino]phenyl]piperidine-1-carboxylate (1.1 g, 2.65 mmol, 52.07% yield) as pale green solid. LCMS: ES+ 413 (M+H). ¹H NMR (400 MHz, DMSO-d₆) δ 10.81 (s, 1H), 7.20 (d, J=8.5 Hz, 1H), 6.96 (t, J=8.7 Hz, 2H), 6.26 (d, J=7.9 Hz, 1H), 4.43-4.37 (m, 1H), 4.09-4.06 (m, 2H), 2.87-2.69 (m, 4H), 2.60-2.55 (m, 1H), 2.10-2.06 (m, 1H), 1.92-1.87 (m, 1H), 1.70-1.67 (m, 2H), 1.57-1.46 (m, 2H), 1.41 (s, 9H).

### Step-4

### Synthesis of 5-[(2,6-dioxo-3-piperidyl)amino]-2-(4-piperidyl)benzonitrile hydrochloride

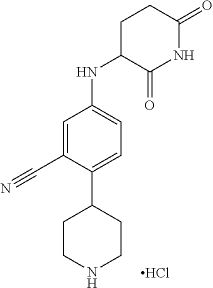 [SMILES: N#Cc1cc(NC2CCC(=O)NC2=O)ccc1C1CCNCC1 ; Cl]

Tert-butyl 4-[2-cyano-4-[(2,6-dioxo-3-piperidyl)amino]phenyl]piperidine-1-carboxylate (120 mg, 290.92 μmol) was dissolved in methanol mixture (3 mL) mL) and Hydrogen chloride solution 4.0M in dioxane (4 M, 727.31 μL) was added. The reaction mixture was heated at 40° C. for 4 hours, and the reaction was complete. The volatiles were evaporated under reduce pressure. The material was submitted to high vacuum, frozen to −78° C. and thawed to afford 5-[(2,6-dioxo-3-piperidyl)amino]-2-(4-piperidyl)benzonitrile hydrochloride (107 mg, 277.51 μmol, 95% yield) as a dense solid. LCMS (ESI+): 313.2 (M+H)

### Synthesis of 3-[3-methyl-4-(4-piperidyl)anilino]piperidine-2,6-dione hydrochloride

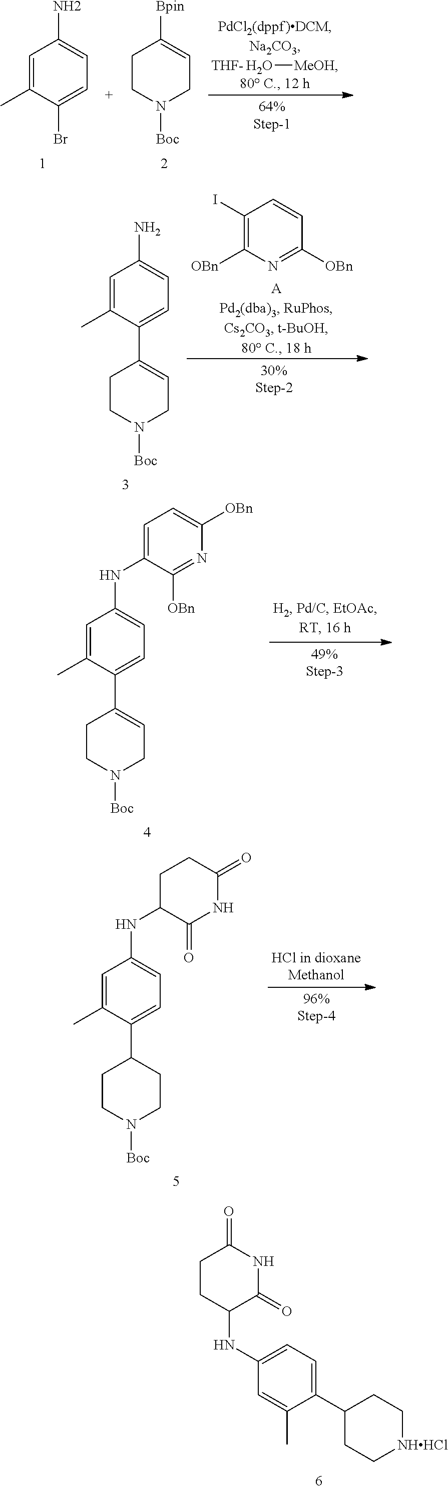

### Step-1: Synthesis of 4-(4-Amino-2-methyl-phenyl)-3,6-dihydro-2H-pyridine-1-carboxylic acid tert-butyl ester

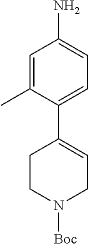 [SMILES: Cc1cc(N)ccc1C1=CCN(C(=O)OC(C)(C)C)CC1]

To a stirred solution of 4-bromo-3-methyl-aniline (5 g, 26.87 mmol) and tert-butyl 4-(4,4,5,5-tetramethyl-1,3,2-dioxaborolan-2-yl)-3,6-dihydro-2H-pyridine-1-carboxylate (9.14 g, 29.56 mmol) in THF (60 mL), water (12 mL) and methanol (24 mL) was added sodium carbonate (6.27 g, 59.12 mmol) and thoroughly purged with argon. PdCl₂(dppf)·CH₂Cl₂ (438.94 mg, 537.49 μmol) was added under inert atmosphere. Resulting mixture was heated at 80° C. for 12 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (15% ethyl acetate-hexane) to afford tert-butyl 4-(4-amino-2-methyl-phenyl)-3,6-dihydro-2H-pyridine-1-carboxylate (5 g, 17.34 mmol, 64.51% yield) as a dense solid. LC MS: ES+ 289 (M+H).

### Step-2: Synthesis of 4-[4-(2,6-Bis-benzyloxy-pyridin-3-ylamino)-2-methyl-phenyl]-3,6-dihydro-2H-pyridine-1-carboxylic acid tert-butyl ester

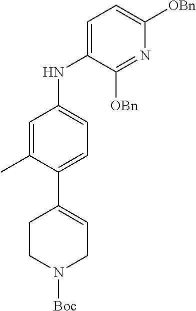 [SMILES: Cc1cc(Nc2ccc(OCc3ccccc3)nc2OCc2ccccc2)ccc1C1=CCN(C(=O)OC(C)(C)C)CC1]

To a stirred solution of tert-butyl 4-(4-amino-2-methyl-phenyl)-3,6-dihydro-2H-pyridine-1-carboxylate (5 g, 17.34 mmol) and 2,6-dibenzyloxy-3-iodo-pyridine (7.96 g, 19.07 mmol) in t-BuOH (80 mL) cesium carbonate (16.95 g, 52.01 mmol) was added. Resulting mixture was degassed with argon and Pd₂(dba)₃ (793.83 mg, 866.90 μmol), RuPhos (809.04 mg, 1.73 mmol) were added under inert atmosphere. Resulting mixture was heated at 100° C. for 18 h. Reaction mixture was diluted with ethyl acetate, filtered through a short pad of celite and washed with ethyl acetate. Combined organic part was washed with water, brine, dried over anhydrous sodium sulphate, filtered and concentrated under reduced pressure. Crude mass was purified by column chromatography (25% ethyl acetate-hexane) to afford tert-butyl 4-[4-[(2,6-dibenzyloxy-3-pyridyl)amino]-2-methyl-phenyl]-3,6-dihydro-2H-pyridine-1-carboxylate (3 g, 5.19 mmol, 29.95% yield) LCMS (ESI+): 578 (M+H⁺).

### Step-3: Synthesis of 4-[4-(2,6-Dioxo-piperidin-3-ylamino)-2-methyl-phenyl]-piperidine-1-carboxylic acid tert-butyl ester

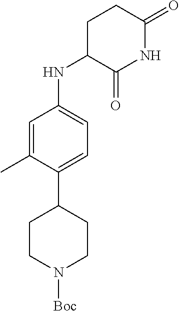 [SMILES: Cc1cc(NC2CCC(=O)NC2=O)ccc1C1CCN(C(=O)OC(C)(C)C)CC1]

To a degassed solution of tert-butyl 4-[4-[(2,6-dibenzyloxy-3-pyridyl)amino]-2-methyl-phenyl]-3,6-dihydro-2H-pyridine-1-carboxylate (3 g, 5.19 mmol) in ethyl acetate (60 mL), 10% Pd—C (50% wet, 3 g) was added. Resulting mixture was stirred at ambient temperature under hydrogen atmosphere at balloon pressure for 16 h. After completion, the reaction mixture was filtered through a short pad of celite, washed with ethyl acetate and concentrated under reduced pressure. The crude residue was purified by silica gel column chromatography (60% ethyl acetate in hexane) to afford tert-butyl 4-[4-[(2,6-dioxo-3-piperidyl)amino]-2-methyl-phenyl]piperidine-1-carboxylate (1020 mg, 2.53 mmol, 48.78% yield) as pale blue solid. LC MS: ES+ 402 (M+H).

### Step 4: Synthesis of 3-[3-methyl-4-(4-piperidyl)anilino]piperidine-2,6-dione hydrochloride

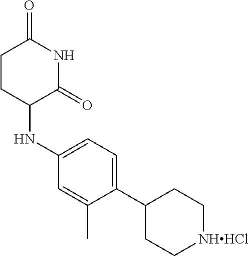

Tert-butyl 4-[4-[(2,6-dioxo-3-piperidyl)amino]-2-methyl-phenyl]piperidine-1-carboxylate (180 mg, 448.32 μmol) was dissolved in methanol (3 mL) and Hydrogen chloride solution 4.0M in dioxane (4 M, 1.12 mL) was added. The reaction mixture was heated at 40° C. for 4 hours, and the reaction was complete. The volatiles were evaporated under reduce pressure. The material was submitted to high vacuum, frozen to −78° C. and thawed to afford 3-[3-methyl-4-(4-piperidyl)anilino]piperidine-2,6-dione hydrochloride (145 mg, 429.19 μmol, 95.73% yield) as a dense solid. LCMS (ESI+): 302.3 (M+H).

### Synthesis of 4-[3-(2,4-dioxohexahydropyrimidin-1-yl)-1-methyl-indazol-6-yl]-3,3-difluoro-piperidine hydrochloride

### Step 1: Synthesis of tert-butyl 3,3-difluoro-4-(trifluoromethylsulfonyloxy)-2,6-dihydropyridine-1-carboxylate

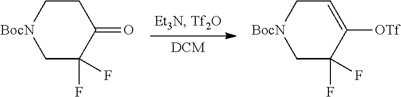 [SMILES: CC(C)(C)OC(=O)N1CCC(=O)C(F)(F)C1 ; CC(C)(C)OC(=O)N1CC=C(OS(=O)(=O)C(F)(F)F)C(F)(F)C1]

N,N-diethylethanamine (3.23 g, 31.9 mmol, 4.44 mL), followed by trifluoromethylsulfonic anhydride (4.50 g, 15.9 mmol, 2.68 mL) were added drop-wise to a stirred solution of tert-butyl 3,3-difluoro-4-oxo-piperidine-1-carboxylate (2.5 g, 10.6 mmol) in dichloromethane (25 mL) at 0° C. The reaction was stirred at ambient temperature for 16 h. Then, the reaction was quenched with aqueous NaHCO₃, and extracted with dichloromethane, washed with brine, dried over Na₂SO₄, and concentrated. The residue was purified by silica gel chromatography (100% hexanes to 4:1 hexanes:ethyl acetate) to yield tert-butyl 3,3-difluoro-4-(trifluoromethylsulfonyloxy)-2,6-dihydropyridine-1-carboxylate (1.2 g, 2.29 mmol, 21% yield). ¹H NMR (400 MHz, Methanol-d4) δ 6.59 (s, 1H), 4.29 (q, J=4.3 Hz, 2H), 4.04 (t, J=11.0 Hz, 2H), 1.51 (s, 9H).

### Step 2: 1-[1-methyl-6-(4,4,5,5-tetramethyl-1,3,2-dioxaborolan-2-yl)indazol-3-yl]hexahydropyrimidine-2,4-dione

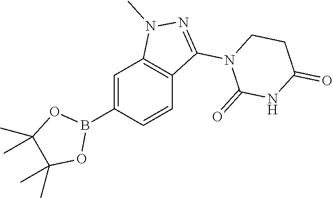 [SMILES: Cn1nc(N2CCC(=O)NC2=O)c2ccc(B3OC(C)(C)C(C)(C)O3)cc21]

Potassium acetate (911 mg, 9.28 mmol) and Pd(dppf)Cl₂ (113 mg, 155 μmol) were added to a solution of 1-(6-bromo-1-methyl-indazol-3-yl)hexahydropyrimidine-2,4-dione (1.0 g, 3.09 mmol) and bis(pinacolato)diboron (1.18 g, 4.64 mmol) in dioxane (15 mL). The mixture was stirred at 85° C. under a nitrogen atmosphere for 16 h. The mixture was cooled to ambient temperature and filtered through a pad of silica gel. The filter cake was washed with ethyl acetate and the filtrate was concentrated under reduced pressure. The residue was purified by silica gel chromatography (100% hexanes to 100% ethyl acetate) to yield 1-[1-methyl-6-(4,4,5,5-tetramethyl-1,3,2-dioxaborolan-2-yl)indazol-3-yl]hexahydropyrimidine-2,4-dione (1.1 g, 2.97 mmol, 96% yield). LCMS (ESI+): 371 (M+H).

### Step 3: tert-Butyl 4-(3-(2,4-dioxotetrahydropyrimidin-1(2H)-yl)-1-methyl-1H-indazol-6-yl)-3,3-difluoro-3,6-dihydropyridine-1(2H)-carboxylate

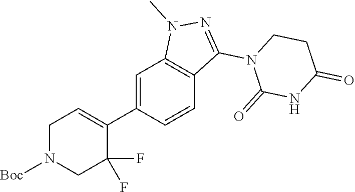 [SMILES: Cn1nc(N2CCC(=O)NC2=O)c2ccc(C3=CCN(C(=O)OC(C)(C)C)CC3(F)F)cc21]

Sodium carbonate (485 mg, 4.57 mmol) was added to a solution of 1-[1-methyl-6-(4,4,5,5-tetramethyl-1,3,2-dioxaborolan-2-yl)indazol-3-yl]hexahydropyrimidine-2,4-dione (677 mg, 1.83 mmol) and tert-butyl 3,3-difluoro-4-(trifluoromethylsulfonyloxy)-2,6-dihydropyridine-1-carboxylate (560 mg, 1.52 mmol) in dioxane (10 mL) and water (2.5 mL) and the solvent was sparged with N₂ gas for 10 min. 1,1′-Bis(Diphenylphosphino)ferrocenepalladium (II) dichloride (111 mg, 152 μmol) was added and the reaction mixture was stirred at 55° C. for 2 h. Then, the reaction mixture was cooled and diluted with water/ethyl acetate. After extraction, organic layer was washed with brine, dried over Na₂SO₄, and concentrated. The residue was purified by silica gel chromatography (100% hexanes to 100% ethyl acetate) to give tert-butyl 4-[3-(2,4-dioxohexahydropyrimidin-1-yl)-1-methyl-indazol-6-yl]-3,3-difluoro-2,6-dihydropyridine-1-carboxylate (480 mg, 1.04 mmol, 68% yield). LCMS (ESI+): 462.2 (M+H)

### Step 4: tert-butyl 4-[3-(2,4-dioxohexahydropyrimidin-1-yl)-1-methyl-indazol-6-yl]-3,3-difluoro-piperidine-1-carboxylate

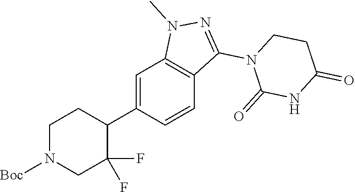 [SMILES: Cn1nc(N2CCC(=O)NC2=O)c2ccc(C3CCN(C(=O)OC(C)(C)C)CC3(F)F)cc21]

Palladium, 10% on carbon (Type 487, dry, 331 mg, 311 μmol) was added to a solution of tert-butyl 4-[3-(2,4-dioxohexahydropyrimidin-1-yl)-1-methyl-indazol-6-yl]-3,3-difluoro-2,6-dihydropyridine-1-carboxylate (478 mg, 1.04 mmol) in methanol (10.3 mL) and the mixture was stirred at ambient temperature under a hydrogen balloon atmosphere. After 24 h, the hydrogen balloon was removed and the mixture was diluted with dichloromethane (20 mL) and the slurry was stirred for additional 24 h. Then, the mixture was filtered through a pad of celite, washed using a solution of dichloromethane/methanol (3:1), and concentrated to afford tert-butyl 4-[3-(2,4-dioxohexahydropyrimidin-1-yl)-1-methyl-indazol-6-yl]-3,3-difluoro-piperidine-1-carboxylate (450 mg, 94% yield). LCMS (ESI+): 408.2 (M-tert-butyl+H).

### Step 5: 4-[3-(2,4-dioxohexahydropyrimidin-1-yl)-1-methyl-indazol-6-yl]-3,3-difluoro-piperidine hydrochloride

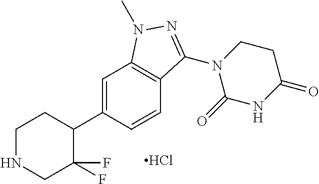 [SMILES: Cn1nc(N2CCC(=O)NC2=O)c2ccc(C3CCNCC3(F)F)cc21 ; Cl]

4-[3-(2,4-dioxohexahydropyrimidin-1-yl)-1-methyl-indazol-6-yl]-3,3-difluoro-piperidine hydrochloride was obtained in quantitative yield from tert-butyl 4-[3-(2,4-dioxohexahydropyrimidin-1-yl)-1-methyl-indazol-6-yl]-3,3-difluoro-piperidine-1-carboxylate using General method B for the removal of the tert-butoxycarbonyl group. LCMS (ESI+): 354.2 (M+H)

### Synthesis of tert-butyl 3,3-difluoro-4-(4-nitrophenyl)piperidine-1-carboxylate, isomer 1 and tert-butyl 3,3-difluoro-4-(4-nitrophenyl)piperidine-1-carboxylate, isomer 2

### Step 1: Synthesis of tert-butyl 4-(4-nitrophenyl)-3-oxo-piperidine-1-carboxylate

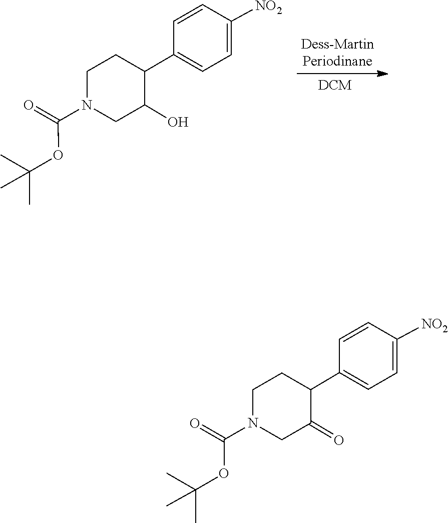 [SMILES: CC(C)(C)OC(=O)N1CCC(c2ccc([N+](=O)[O-])cc2)C(O)C1 ; CC(C)(C)OC(=O)N1CCC(c2ccc([N+](=O)[O-])cc2)C(=O)C1]

tert-Butyl 3-hydroxy-4-(4-nitrophenyl)piperidine-1-carboxylate (19.5 g, 60.5 mmol) (CAS #1232788-17-8) was dissolved in dichloromethane (200 mL) and cooled to 0° C. Dess-Martin Periodinane (38.5 g, 90.7 mmol) was added portion-wise. Internal temperature increased from 0 to 2.2° C. during the initial addition. The reaction solution was stirred at that temperature for 2 h and stirring was continued while the temperature gradually climbed up to ambient temperature. After 17 h, the reaction solution became a slurry due to some solvent evaporation. Dichloromethane (100 mL) was added, followed by Dess-Martin Periodinane (8.3 g, 19.6 mmol) at 16° C. and the reaction was stirred for 17 h. The reaction solution was cooled back down to 4° C. Saturated NaHCO₃ solution (250 mL) was carefully added, followed by sodium thiosulfate pentahydrate (13.8 g, 48.4 mmol) dissolved in 175 mL of water. The mixture was diluted with dichloromethane (150 mL). The resulting precipitate was removed by filtration and the cake was washed with dichloromethane (75 mL×3). The filtrate was separated into layers and the organic layer was dried over Na₂SO₄, filtered and concentrated to afford tert-butyl 4-(4-nitrophenyl)-3-oxo-piperidine-1-carboxylate (19.4 g, 60.5 mmol, quantitative yield). LCMS (ESI+): 354.1 (M+Na)/221.0 (M-Boc+H).

### Step 2: Synthesis of tert-butyl 3,3-difluoro-4-(4-nitrophenyl)piperidine-1-carboxylate

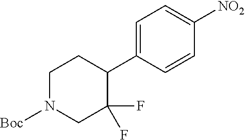 [SMILES: CC(C)(C)OC(=O)N1CCC(c2ccc([N+](=O)[O-])cc2)C(F)(F)C1]

tert-Butyl 4-(4-nitrophenyl)-3-oxo-piperidine-1-carboxylate (3.78 g, 11.8 mmol) was dissolved in dichloromethane (40 mL) and the solution was cooled to 0° C. DAST (3.80 g, 23.6 mmol, 3.12 mL) was added slowly via a syringe. The reaction mixture was warmed slowly to room temperature while it was stirred overnight. The reaction solution was cooled to −1.3° C. and saturated aqueous NaHCO₃ (100 mL) was added carefully via an addition funnel (exothermic). Internal temperature was maintained below 18° C. during the addition. The reaction mixture was diluted with ethyl acetate (80 mL) and warmed up to ambient temperature. The layers were separated and the aqueous layer was washed with ethyl acetate (80 mL). The combined organics were washed with aqueous 18% NaCl solution and concentrated. The residue was purified by silica gel chromatography (gradient: 10-30% ethyl acetate in hexanes to afford tert-butyl 3,3-difluoro-4-(4-nitrophenyl)piperidine-1-carboxylate (2.50 g, 62% yield) LCMS (ESI+): 280.2 (M-tert-Butyl+H)/243.1 (M-Boc+H).

### Step 3: Chiral separation to obtain tert-butyl 3,3-difluoro-4-(4-nitrophenyl)piperidine-1-carboxylate, isomer 1 and tert-butyl 3,3-difluoro-4-(4-nitrophenyl)piperidine-1-carboxylate, Isomer 2

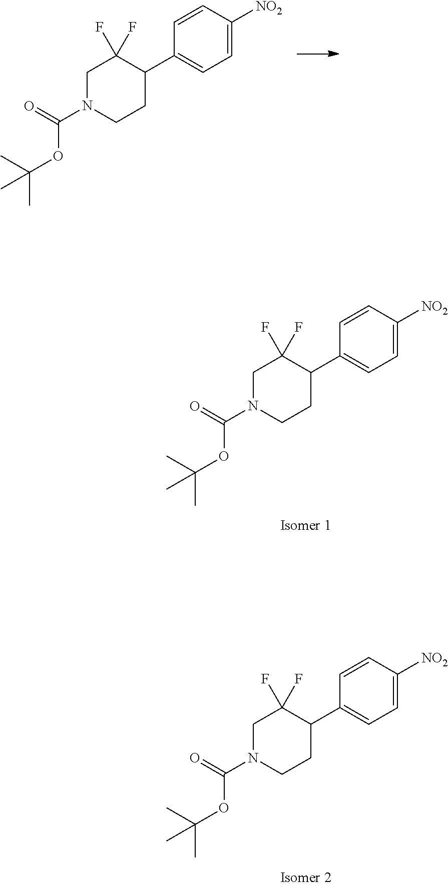 [SMILES: CC(C)(C)OC(=O)N1CCC(c2ccc([N+](=O)[O-])cc2)C(F)(F)C1 ; CC(C)(C)OC(=O)N1CCC(c2ccc([N+](=O)[O-])cc2)C(F)(F)C1 ; CC(C)(C)OC(=O)N1CCC(c2ccc([N+](=O)[O-])cc2)C(F)(F)C1]

Racemic tert-butyl 3,3-difluoro-4-(4-nitrophenyl)piperidine-1-carboxylate (2.49 g) was subjected to a Chiral SFC separation, under the following conditions:
Column: ChiralPak IC-H 21×250 mm
Mobile Phase: 10% 2-propanol in carbon dioxide.
Flow rate: 70 mL/min
Detection: 220 nm UV
Pressure: 100 bar

The first eluting set of fractions was evaporated under reduced pressure to afford tert-butyl 3,3-difluoro-4-(4-nitrophenyl)piperidine-1-carboxylate, isomer 1 (800 mg, 32% yield, Rt=1.74 min, >99% enantiomeric excess) LCMS: 280.2 (M-tBu+H)/243.1 (M-Boc+H).

The second eluting set of fractions was evaporated under reduced pressure to afford tert-butyl 3,3-difluoro-4-(4-nitrophenyl)piperidine-1-carboxylate, isomer 2 (800 mg, 32% yield, Rt=2.31 min., 99.6% enantiomeric excess). LCMS 280.2 (M-tert-Butyl+H)/243.1 (M-Boc+H).

The enantiomeric excess of the purified enantiomers was determined using the following analytical SFC method.
Column: ChiralPak IC-H 4.6×100 mm
Mobile phase: 10% iso-propanol in carbon dioxide
Flow rate: 4 mL/min
Pressure: 100 bar

### Synthesis of 3-[4-[3,3-difluoro-4-piperidyl]anilino]piperidine-2,6-dione dihydrochloride, isomer 1

### Step 1: tert-butyl-4-(4-aminophenyl)-3,3-difluoro-piperidine-1-carboxylate, Isomer 1

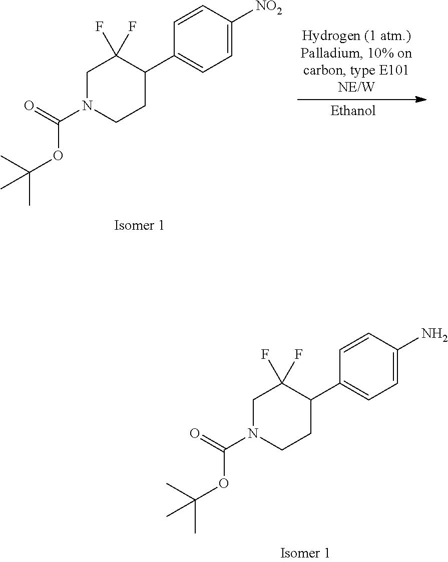 [SMILES: CC(C)(C)OC(=O)N1CCC(c2ccc([N+](=O)[O-])cc2)C(F)(F)C1 ; CC(C)(C)OC(=O)N1CCC(c2ccc(N)cc2)C(F)(F)C1]

tert-Butyl 3,3-difluoro-4-(4-nitrophenyl)piperidine-1-carboxylate, isomer 1 (0.8 g, 2.34 mmol) was dissolved in ethanol (12 mL) and the solution was degassed with nitrogen. Palladium, 10% on carbon, type E101 NE/W (125 mg, 1.17 mmol) was then added. After degassing again with nitrogen couple, the reaction mixture was stirred under a hydrogen balloon atmosphere for 16 h. The reaction mixture was filtered through a pad of celite, washed with ethyl acetate (12 mL×3) and the filtrate was concentrated to yield tert-butyl-4-(4-aminophenyl)-3,3-difluoro-piperidine-1-carboxylate, isomer 1 (722 mg, 98% yield). LCMS (ESI+): 257 (M-tBu+H)

### Step 2: tert-butyl (4S)-4-[4-[(2,6-dioxo-3-piperidyl)amino]phenyl]-3,3-difluoro-piperidine-1-carboxylate, Isomer 1

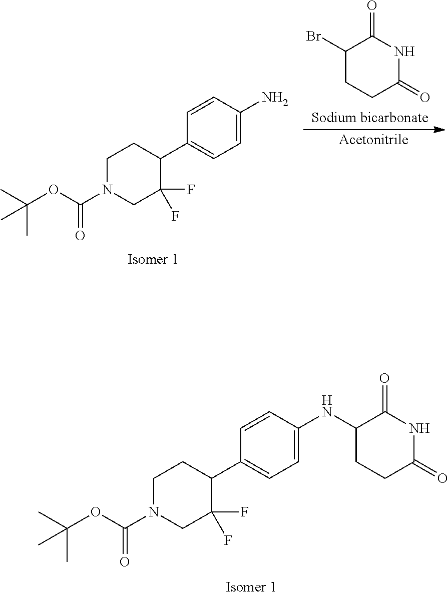 [SMILES: CC(C)(C)OC(=O)N1CCC(c2ccc(N)cc2)C(F)(F)C1 ; O=C1CCC(Br)C(=O)N1 ; CC(C)(C)OC(=O)N1CCC(c2ccc(NC3CCC(=O)NC3=O)cc2)C(F)(F)C1]

Acetonitrile (3.5 mL) was added to tert-Butyl 4-(4-aminophenyl)-3,3-difluoro-piperidine-1-carboxylate, isomer 1 (520 mg, 1.66 mmol), 3-bromopiperidine-2,6-dione (478 mg, 2.49 mmol) and NaHCO₃(418 mg, 4.98 mmol) in a vial. The reaction mixture was heated to 70° C. for 45 h. 3-bromopiperidine-2,6-dione (92 mg, 0.28 equiv) and NaHCO₃(110 mg, 0.78 equiv) were added and heating was continued for a further 72 h, at which point, the reaction was cooled to ambient temperature and water (18 mL) was slowly added. The mixture was stirred for 4 h, then the precipitate was collected by filtration, washing with water (10 mL×3), then with 9:1 hexane:ethyl acetate (5 mL×3). The filter cake was dried under vacuum to afford tert-butyl 4-[4-[(2,6-dioxo-3-piperidyl)amino]phenyl]-3,3-difluoro-piperidine-1-carboxylate, isomer 1 (577 mg, 78% yield) as a green solid. LCMS (ESI+): 446 (M+Na).

### Step 3: 3-[4-[3,3-difluoro-4-piperidyl]anilino]piperidine-2,6-dione dihydrochloride, Isomer 1

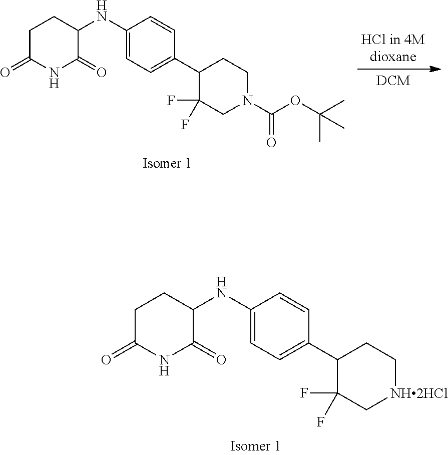

Tert-butyl 4-[4-[(2,6-dioxo-3-piperidyl)amino]phenyl]-3,3-difluoro-piperidine-1-carboxylate, isomer 1 (300 mg, 709 μmol), was dissolved in dichloromethane (3.4 mL), and hydrogen chloride (4M in 1,4-dioxane, 850 μL, 3.4 mmol) was added under stirring. After 1 hour, the reaction mixture was concentrated to afford 3-[4-[3,3-difluoro-4-piperidyl]anilino]piperidine-2,6-dione dihydrochloride, isomer 1 in quantitative yield. LCMS (ESI+): 324 (M+H).

### Synthesis of 3-[4-[3,3-difluoro-4-piperidyl]anilino]piperidine-2,6-dione dihydrochloride, Isomer 2

### Step 1: Synthesis of tert-butyl-4-(4-aminophenyl)-3,3-difluoro-piperidine-1-carboxylate, Isomer 2

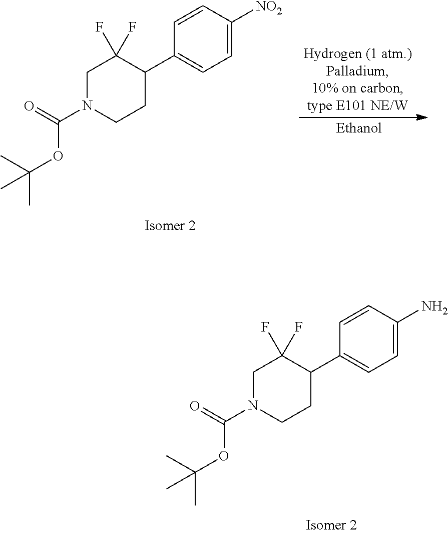 [SMILES: CC(C)(C)OC(=O)N1CCC(c2ccc([N+](=O)[O-])cc2)C(F)(F)C1 ; CC(C)(C)OC(=O)N1CCC(c2ccc(N)cc2)C(F)(F)C1]

Tert-butyl 3,3-difluoro-4-(4-nitrophenyl)piperidine-1-carboxylate, isomer 2 (800 mg, 2.34 mmol) was dissolved in Ethanol (12 mL). The solution was evacuated and back-filled with nitrogen couple times. Palladium, 10% on carbon, type E101 NE/W (124.35 mg, 1.17 mmol) was then added. After evacuated and backfilled with nitrogen couple more times, the reaction mixture was subjected to hydrogenation (H2 balloon) at ambient temperature for 16 hours. The reaction mixture was filtered through a pad of Celite. The celite cake was washed with ethyl acetate (12 mL×3). The filtrate was concentrated in vacuo and further dried under vacuum to yield a semi-solid (oily) upon standing; tert-butyl-4-(4-aminophenyl)-3,3-difluoro-piperidine-1-carboxylate, isomer 2 (724 mg, 94% yield). LCMS (ESI+): 257.1 (M-tBu+H)

### Step 2: Synthesis of 4-[4-[(2,6-dioxo-3-piperidyl)amino]phenyl]-3,3-difluoro-piperidine-1-carboxylate, Isomer 2

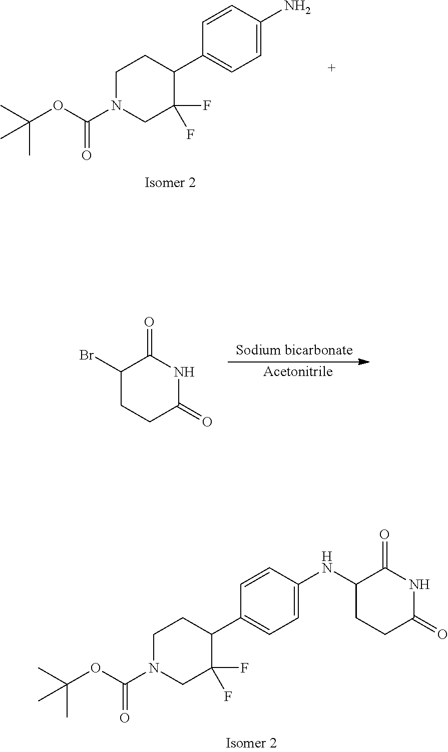 [SMILES: CC(C)(C)OC(=O)N1CCC(c2ccc(N)cc2)C(F)(F)C1 ; O=C1CCC(Br)C(=O)N1 ; CC(C)(C)OC(=O)N1CCC(c2ccc(NC3CCC(=O)NC3=O)cc2)C(F)(F)C1]

To a vial was added tert-butyl 4-(4-aminophenyl)-3,3-difluoro-piperidine-1-carboxylate, isomer 2 (721.54 mg, 2.31 mmol), 3-bromopiperidine-2,6-dione (665.32 mg, 3.47 mmol), and Sodium bicarbonate (582.19 mg, 6.93 mmol, 269.53 Added Acetonitrile (5 mL). Reaction mixture was warmed to 70° C. (block temperature) overnight. After 48 hours, added additional amount of 3-bromopiperidine-2,6-dione (129 mg, 0.28 equiv), NaHCO₃(129 mg, 0.66 equiv). After another 72 hours, cooled to ambient temperature. Water (25 mL) was added slowly. Stirred at ambient temperature for couple hours. Reaction mixture was filtered to collect solid. Washed with water (12 mL×3), 9:1 hexane:ethyl acetate (5 mL×2), and dried under vacuum to afford tert-butyl 4-[4-[(2,6-dioxo-3-piperidyl)amino]phenyl]-3,3-difluoro-piperidine-1-carboxylate, isomer 2 (838 mg, 81.4% yield) as a green solid. LCMS (ESI+): 446.4 (M+Na)

### Step 3: Synthesis of 3-[4-[3,3-difluoro-4-piperidyl]anilino]piperidine-2,6-dione dihydrochloride, Isomer 2

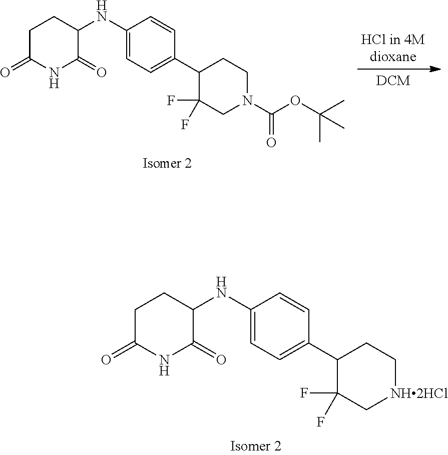

Tert-butyl 4-[4-[(2,6-dioxo-3-piperidyl)amino]phenyl]-3,3-difluoro-piperidine-1-carboxylate, isomer 2 (300 mg, 708.5 μmol), was dissolved in Dichloromethane (3.4 mL), and hydrogen chloride (4M in 1,4-dioxane, 850 μL, 3.4 mmol) was added under stirring. After 1 hour, the reaction mixture was evaporated to dryness under reduced pressure to afford 3-[4-[3,3-difluoro-4-piperidyl]anilino]piperidine-2,6-dione dihydrochloride, isomer 2 in quantitative yield. LCMS (ESI+): 324.1 (M+H).

### Synthesis of 2-[1-[4-[(2,6-dioxo-3-piperidyl)amino]-2-(trifluoromethyl)phenyl]-4-hydroxy-4-piperidyl]acetic acid

### Step 1: tert-butyl 2-[4-hydroxy-1-[4-nitro-2-(trifluoromethyl)phenyl]-4-piperidyl]acetate

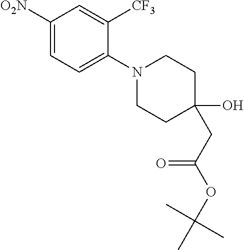 [SMILES: CC(C)(C)OC(=O)CC1(O)CCN(c2ccc([N+](=O)[O-])cc2C(F)(F)F)CC1]

Lithium diisopropylamide (0.7 M in THF, 54 mL, 37.82 mmol) was added dropwise over a period of 10 min to a stirred solution of tert-butyl acetate (1.76 g, 15.1 mmol, 2.04 mL) in dry THF (40 ml) at −78° C. The reaction mixture was stirred for 1 h. 1-[4-nitro-2-(trifluoromethyl)phenyl]piperidin-4-one (4.36 g, 15.1 mmol) dissolved in THF (20 ml) was added slowly. The reaction was stirred for 1 h at −78° C. The reaction was quenched with aqueous ammonium chloride solution at −78° C. and the mixture was warmed to ambient temperature and extracted with ethyl acetate. The organic layer was washed with brine and concentrated to afford a residue which was used without further purification. LCMS (ESI−): m/z 403.1 [M−H⁺].

### Step 2: tert-butyl 2-[1-[4-amino-2-(trifluoromethyl)phenyl]-4-hydroxy-4-piperidyl]acetate

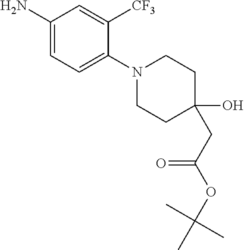 [SMILES: CC(C)(C)OC(=O)CC1(O)CCN(c2ccc(N)cc2C(F)(F)F)CC1]

A stirred solution of tert-butyl 2-[4-hydroxy-1-[4-nitro-2-(trifluoromethyl)phenyl]-4-piperidyl]acetate (2 g, 4.95 mmol) in a ethyl acetate (40 mL) was purged with nitrogen for 5 min. Pd/C, 10% on dry basis (1.05 g, 9.89 mmol) was added to the reaction mixture. The reaction mixture was placed under an hydrogen atmosphere (balloon). The reaction mixture was stirred for 4 h. The reaction mixture was filtered through a celite bed by flushing with a dichloromethane: ethyl acetate mixture (1:1, 500 mL). The filtrate was concentrated under reduced pressure to afford brownish solid was dissolved in dichloromethane (20 mL) and dry packed on silica under reduced pressure. The compound was purified by silica gel (230-400 mesh) column chromatography using a ethyl acetate:petroleum ether. The pure fractions were combined and concentrated under reduced pressure to afford pure reddish-brown solid tert-butyl 2-[1-[4-amino-2-(trifluoromethyl)phenyl]-4-hydroxy-4-piperidyl]acetate (1.1 g, 1.96 mmol, 40% yield). LCMS (m/z: 375.2 (M+H⁺)).

### Step 3: tert-butyl 2-[1-[4-[(2,6-dioxo-3-piperidyl)amino]-2-(trifluoromethyl)phenyl]-4-hydroxy-4-piperidyl]acetate

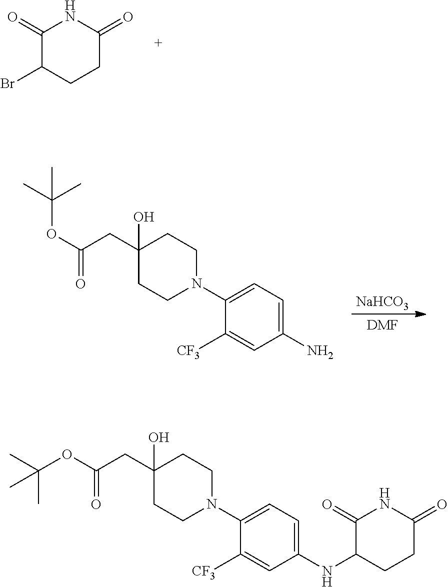

To a stirred solution of tert-butyl 2-[1-[4-amino-2-(trifluoromethyl)phenyl]-4-hydroxy-4-piperidyl]acetate (1.1 g, 2.94 mmol) and 3-bromopiperidine-2,6-dione (846.21 mg, 4.41 mmol) in DMF (10 mL) was added sodium bicarbonate (740.45 mg, 8.81 mmol) at room temperature, after 10 min the temperature of the reaction was raised to 60° C. and continued the reaction about 12 hr. The reaction mixture was diluted with ice-cold water (20 mL) and extracted by ethyl acetate (2*100 mL), washed with brine (10 mL), dried over anhydrous Na₂SO₄, filtered and concentrated under reduced pressure. The crude product was purified using silica gel chromatography using a 10% to 100% Ethyl acetate in Petroleum ether eluent gradient. The pure fractions were combined and concentrated under reduced pressure to afford tert-butyl 2-[1-[4-[(2,6-dioxo-3-piperidyl)amino]-2-(trifluoromethyl)phenyl]-4-hydroxy-4-piperidyl]acetate (300 mg, 468.45 μmol, 16% yield) as a brownish-green solid. LCMS (ESI+) m/z: 486.2 (M+H⁺).

### Step 4: Synthesis of 2-[1-[4-[(2,6-dioxo-3-piperidyl)amino]-2-(trifluoromethyl)phenyl]-4-hydroxy-4-piperidyl]acetic acid

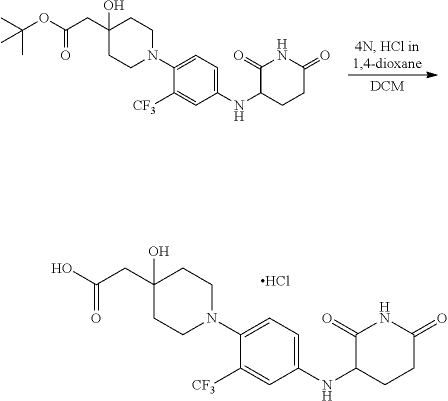

To a stirred solution of tert-butyl 2-[1-[4-[(2,6-dioxo-3-piperidyl)amino]-2-(trifluoromethyl)phenyl]-4-hydroxy-4-piperidyl]acetate (620 mg, 1.28 mmol) in dichloromethane (3 mL) was added hydrogen chloride (4M in 1,4-dioxane, 0.32 mL, 6.39 mmol) dropwise at 0° C. under nitrogen atmosphere, it was stirred for 6 h at room temperature. The reaction mixture was distilled under vacuum and triturated with diethyl ether, decanted the diethyl ether then dried to afford 2-[1-[4-[(2,6-dioxo-3-piperidyl)amino]-2-(trifluoromethyl)phenyl]-4-hydroxy-4-piperidyl]acetic acid (345 mg, 661 μmol, 52% yield) as a green colored solid. LCMS (m/z: 430.1 (M+H))

### General Procedure C for the Alkylation of Intermediates with tert-butyl 2-bromoacetate

### Synthesis of tert-butyl 2-[4-[4-[(2,6-dioxo-3-piperidyl)amino]phenyl]-1-piperidyl]acetate

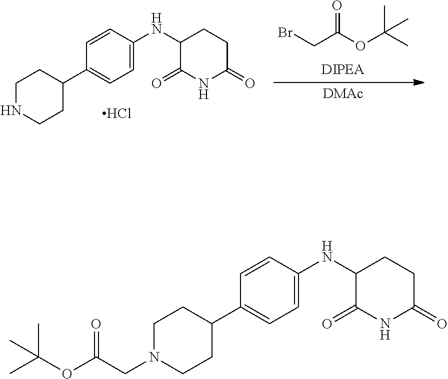

3-[4-(4-piperidyl)anilino]piperidine-2,6-dione hydrochloride (1 g, 3.09 mmol) was dissolved in N,N-dimethylacetamide (15 mL) and N,N-diisopropylethylamine (1.60 g, 12.4 mmol, 2.15 mL) was added. The mixture was cooled to 0° C., and tert-butyl 2-bromoacetate (663 mg, 3.40 mmol, 498 μL) was added. The mixture was stirred at 0° C. for 4 h. The reaction was diluted with ethyl acetate and washed with saturated sodium bicarbonate and brine. The organic layer was concentrated and purified by silica gel chromatography (0-10% Methanol in dichloromethane) to yield tert-butyl 2-[4-[4-[(2,6-dioxo-3-piperidyl)amino]phenyl]-1-piperidyl]acetate (0.84 g, 2.09 mmol, 68% yield) as a white solid. LCMS (ESI+): 402.2 (M+H⁺)

The following compounds were synthesized using General procedure C, as that used for the synthesis of tert-butyl 2-[4-[4-[(2,6-dioxo-3-piperidyl)amino]phenyl]-1-piperidyl]acetate from 3-[4-(4-piperidyl)anilino]piperidine-2,6-dione hydrochloride.

| | | LCMS | % |
|---|---|---|---|
| | | (ESI+) | Yield |
| Starting material | Product | m/z | |
| 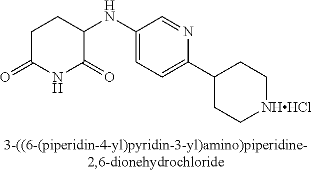 | 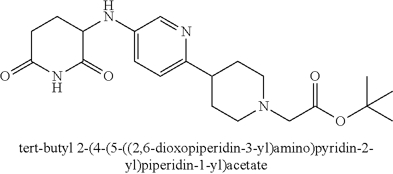 [SMILES: CC(C)(C)OC(=O)CN1CCC(c2ccc(NC3CCC(=O)NC3=O)cn2)CC1] | 347.2 [M − tBu + 1] | 73% |
| 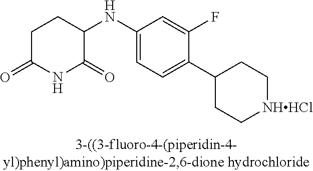 | 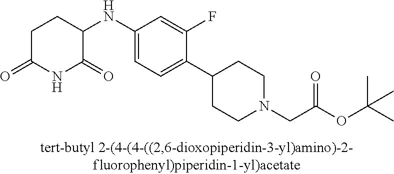 [SMILES: CC(C)(C)OC(=O)CN1CCC(c2ccc(NC3CCC(=O)NC3=O)cc2F)CC1] | 420.2 (M + 1) | 72% |
| 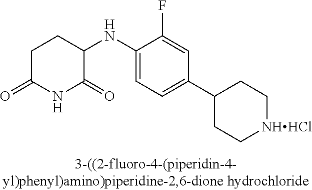 | 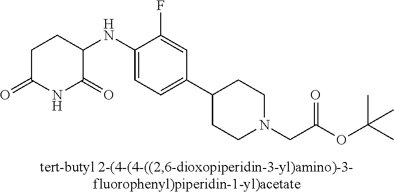 [SMILES: CC(C)(C)OC(=O)CN1CCC(c2ccc(NC3CCC(=O)NC3=O)c(F)c2)CC1] | 420.2 (M + 1) | 65% |
| 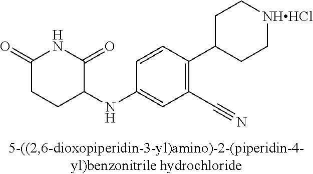 | 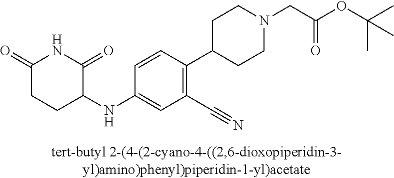 [SMILES: CC(C)(C)OC(=O)CN1CCC(c2ccc(NC3CCC(=O)NC3=O)cc2C#N)CC1] | 427 (M + H) | 82% |
| 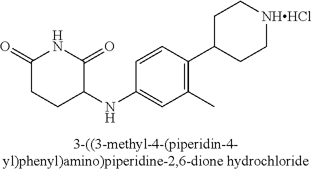 | 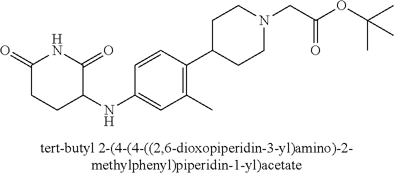 [SMILES: Cc1cc(NC2CCC(=O)NC2=O)ccc1C1CCN(CC(=O)OC(C)(C)C)CC1] | 416.3 (M + H) | 75% |
| 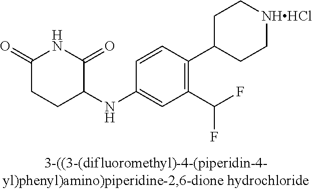 | 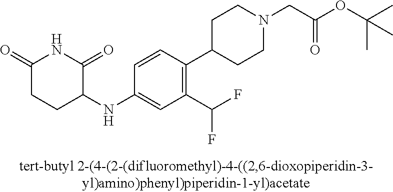 [SMILES: CC(C)(C)OC(=O)CN1CCC(c2ccc(NC3CCC(=O)NC3=O)cc2C(F)F)CC1] | 452.2 (M + H) | 68% |
| 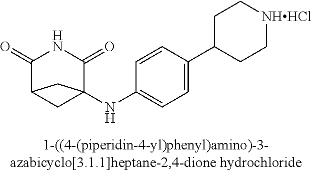 | 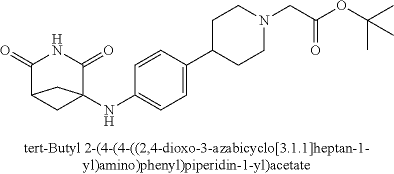 [SMILES: CC(C)(C)OC(=O)CN1CCC(c2ccc(NC34CC(C3)C(=O)NC4=O)cc2)CC1] | 414.51 [M + H] | 57% |
| 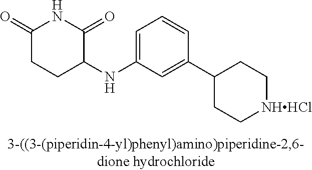 | 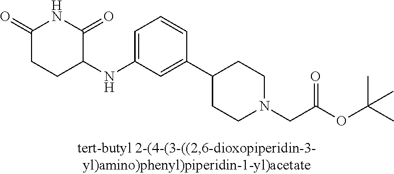 [SMILES: CC(C)(C)OC(=O)CN1CCC(c2cccc(NC3CCC(=O)NC3=O)c2)CC1] | 402.1 [M + H] | 24% |
| 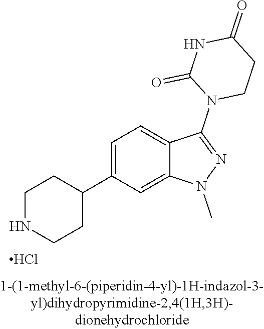 | 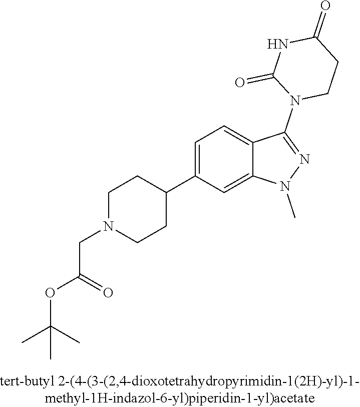 [SMILES: Cn1nc(N2CCC(=O)NC2=O)c2ccc(C3CCN(CC(=O)OC(C)(C)C)CC3)cc21] | 442.3 (M + H) | 75% |
| 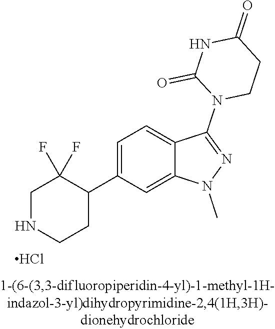 | 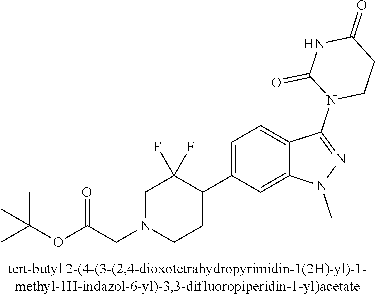 [SMILES: Cn1nc(N2CCC(=O)NC2=O)c2ccc(C3CCN(CC(=O)OC(C)(C)C)CC3(F)F)cc21] | 478.5 (M + H) | 47% |
| 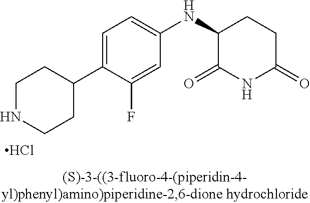 [SMILES: O=C1CC[C@H](Nc2ccc(C3CCNCC3)c(F)c2)C(=O)N1 ; Cl] | 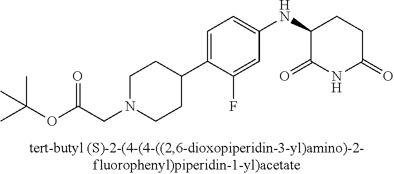 [SMILES: CC(C)(C)OC(=O)CN1CCC(c2ccc(N[C@H]3CCC(=O)NC3=O)cc2F)CC1] | 420.0 (M + H) | 68% |
| 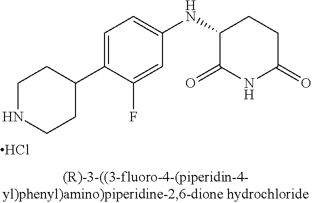 [SMILES: O=C1CC[C@@H](Nc2ccc(C3CCNCC3)c(F)c2)C(=O)N1 ; Cl] | 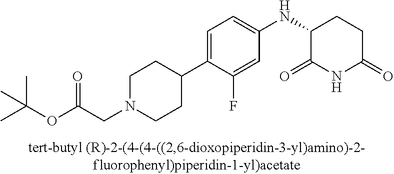 [SMILES: CC(C)(C)OC(=O)CN1CCC(c2ccc(N[C@@H]3CCC(=O)NC3=O)cc2F)CC1] | 420.2 (M + H) | 86.1% |
| 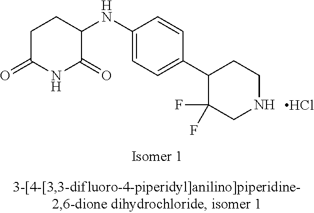 [SMILES: O=C1CCC(Nc2ccc(C3CCNCC3(F)F)cc2)C(=O)N1 ; Cl] | 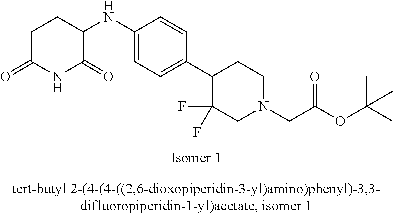 [SMILES: CC(C)(C)OC(=O)CN1CCC(c2ccc(NC3CCC(=O)NC3=O)cc2)C(F)(F)C1] | 438.2 (M + H) | 84% |
| 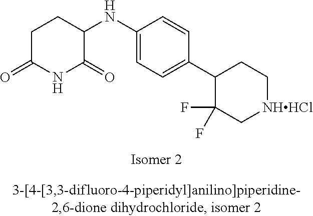 | 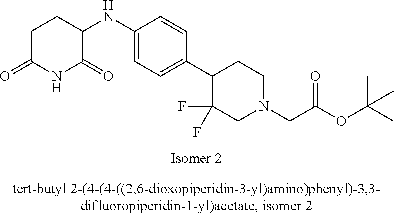 [SMILES: CC(C)(C)OC(=O)CN1CCC(c2ccc(NC3CCC(=O)NC3=O)cc2)C(F)(F)C1] | 438.2 (M + H) | 87% |

### General Procedure D for the tert-butyl ester Cleavage of Intermediates: 2-[4-[4-[(2,6-dioxo-3-piperidyl)amino]phenyl]-1-piperidyl]acetic acid, trifluoroacetic acid salt

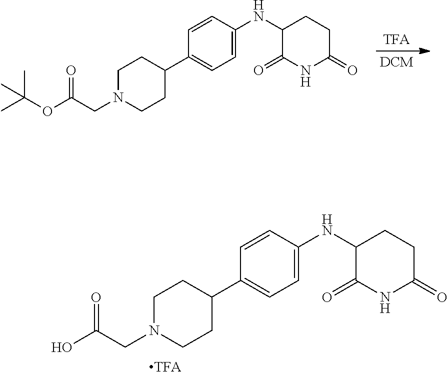

tert-Butyl 2-[4-[4-[(2,6-dioxo-3-piperidyl)amino]phenyl]-1-piperidyl]acetate was dissolved in dichloromethane (5 mL) and TFA (1.61 mL, 20.9 mmol) was added. The reaction mixture was heated at 40° C. for 4 h, and the reaction was complete. The volatiles were evaporated under reduce pressure. The material was frozen to −78° C., submitted to high vacuum, and thawed to afford a dense solid. The solid was re-dissolved in methanol:dicloromethane (1:4), MTBE was added dropwise, until a precipitate formed. The suspension was submitted to sonication, and the solid was filtered under suction. The green solid was collected by filtration to afford 2-[4-[4-[(2,6-dioxo-3-piperidyl)amino]phenyl]-1-piperidyl]acetic acid, trifluoroacetic acid salt (0.95 g, 2.07 mmol, 97% yield). LCMS (ESI+): 346.4 (M+H⁺)

The following intermediates were synthesized from the appropriate starting materials using general procedure D for 2-[4-[4-[(2,6-dioxo-3-piperidyl)amino]phenyl]-1-piperidyl]acetic acid, trifluoroacetic acid salt synthesis.

| | | | LC |
|---|---|---|---|
| | | | MS |
| | | | ESI+ |
| Starting material | Product | Yield | m/z |
| 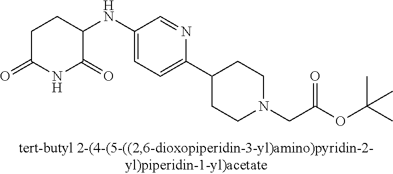 [SMILES: CC(C)(C)OC(=O)CN1CCC(c2ccc(NC3CCC(=O)NC3=O)cn2)CC1] | 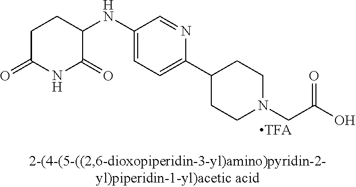 [SMILES: O=C(O)CN1CCC(c2ccc(NC3CCC(=O)NC3=O)cn2)CC1] | 72% | 347.2 (M + H) |
| 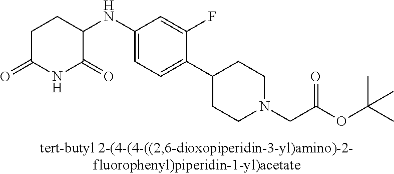 [SMILES: CC(C)(C)OC(=O)CN1CCC(c2ccc(NC3CCC(=O)NC3=O)cc2F)CC1] | 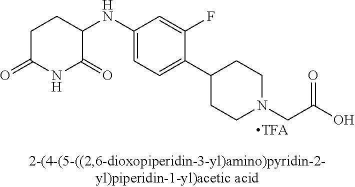 [SMILES: O=C(O)CN1CCC(c2ccc(NC3CCC(=O)NC3=O)cc2F)CC1] | >98% | 364.2 (M + H) |
| 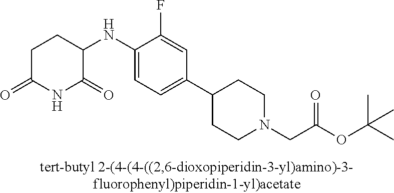 [SMILES: CC(C)(C)OC(=O)CN1CCC(c2ccc(NC3CCC(=O)NC3=O)c(F)c2)CC1] | 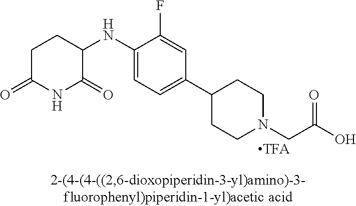 [SMILES: O=C(O)CN1CCC(c2ccc(NC3CCC(=O)NC3=O)c(F)c2)CC1] | >98% | 364.5 (M + H) |
| 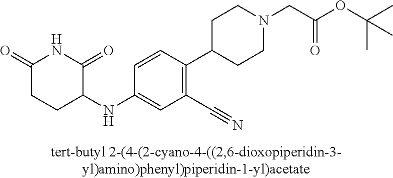 [SMILES: CC(C)(C)OC(=O)CN1CCC(c2ccc(NC3CCC(=O)NC3=O)cc2C#N)CC1] | 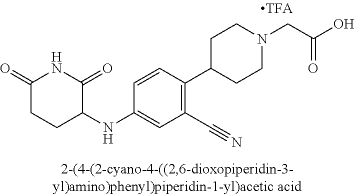 [SMILES: N#Cc1cc(NC2CCC(=O)NC2=O)ccc1C1CCN(CC(=O)O)CC1] | >98% | 371.2 (M + H) |
| 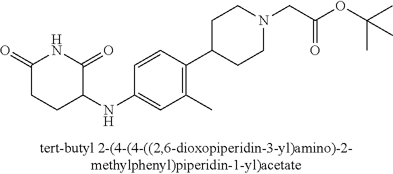 [SMILES: Cc1cc(NC2CCC(=O)NC2=O)ccc1C1CCN(CC(=O)OC(C)(C)C)CC1] | 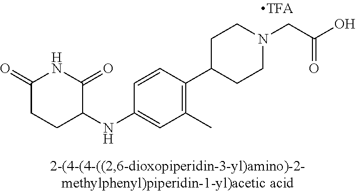 [SMILES: Cc1cc(NC2CCC(=O)NC2=O)ccc1C1CCN(CC(=O)O)CC1] | 86% | 360.3 (M + H) |
| 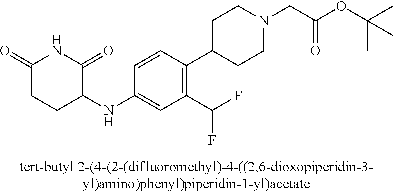 | 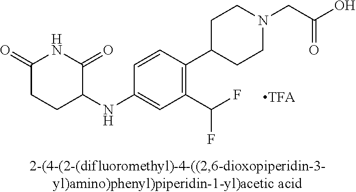 [SMILES: O=C(O)CN1CCC(c2ccc(NC3CCC(=O)NC3=O)cc2C(F)F)CC1] | 94% | 369.2 (M + H) |
| 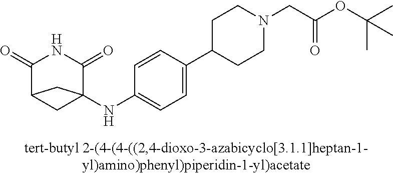 [SMILES: CC(C)(C)OC(=O)CN1CCC(c2ccc(NC34CC(C3)C(=O)NC4=O)cc2)CC1] | 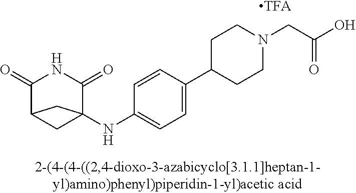 [SMILES: O=C(O)CN1CCC(c2ccc(NC34CC(C3)C(=O)NC4=O)cc2)CC1 ; "·TFA"] | 73% | 358.1 (M + H) |
| 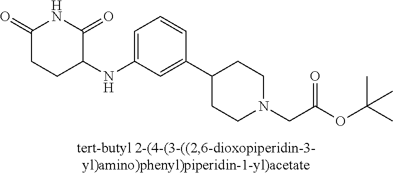 [SMILES: CC(C)(C)OC(=O)CN1CCC(c2cccc(NC3CCC(=O)NC3=O)c2)CC1] | 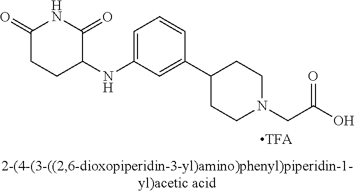 [SMILES: O=C(O)CN1CCC(c2cccc(NC3CCC(=O)NC3=O)c2)CC1 ; "·TFA"] | 91% | 346.7 (M + H) |
| 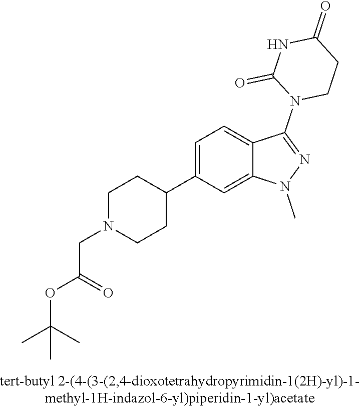 [SMILES: Cn1nc(N2CCC(=O)NC2=O)c2ccc(C3CCN(CC(=O)OC(C)(C)C)CC3)cc21] | 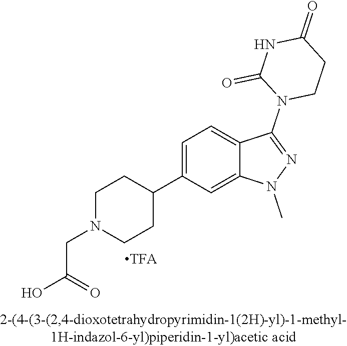 [SMILES: Cn1nc(N2CCC(=O)NC2=O)c2ccc(C3CCN(CC(=O)O)CC3)cc21 ; "·TFA"] | 82% | 386.1 (M + H) |
| 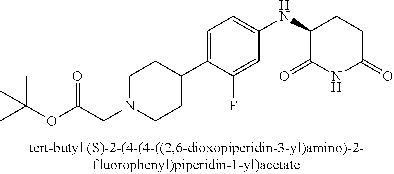 [SMILES: CC(C)(C)OC(=O)CN1CCC(c2ccc(N[C@H]3CCC(=O)NC3=O)cc2F)CC1] | 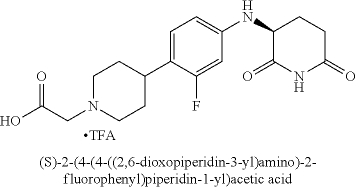 [SMILES: O=C(O)CN1CCC(c2ccc(N[C@H]3CCC(=O)NC3=O)cc2F)CC1 ; "·TFA"] | 93% | 364.1 [M + 1] |
| 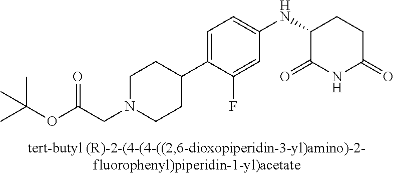 [SMILES: CC(C)(C)OC(=O)CN1CCC(c2ccc(N[C@@H]3CCC(=O)NC3=O)cc2F)CC1] | 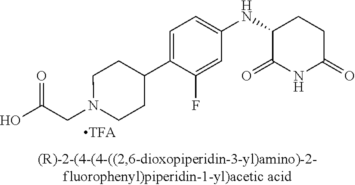 [SMILES: O=C(O)CN1CCC(c2ccc(N[C@@H]3CCC(=O)NC3=O)cc2F)CC1 ; "·TFA"] | 79% | 364.1 [M + 1] |

The enantiomeric excess for intermediate (S)-2-(4-(4-((2,6-dioxopiperidin-3-yl)amino)-2-fluorophenyl)piperidin-1-yl)acetic acid was measured at >99.9% ee (Rt=2.11 min.) using the following SFC method:
Column: Lux A1
Eluent: 50% Isopropanol with 0.5% isopropyl amine in CO₂ (isochratic)
Pressure: 100 bar
Temperature: 35° C.
Run time: 7 min

The enantiomeric excess for intermediate (R)-2-(4-(4-((2,6-dioxopiperidin-3-yl)amino)-2-fluorophenyl)piperidin-1-yl)acetic acid was measured at 98% ee (Rt=3.93 min.) using the following SFC method:
Column: Lux A1
Eluent: 50% Isopropanol with 0.5% isopropyl amine in CO₂ (isochratic)
Pressure: 100 bar
Temperature: 35° C.
Run time: 7 min

### General Procedure E: Synthesis of 2-[4-[4-[(2,6-dioxo-3-piperidyl)amino]phenyl]-1-piperidyl]acetic acid hydrochloride

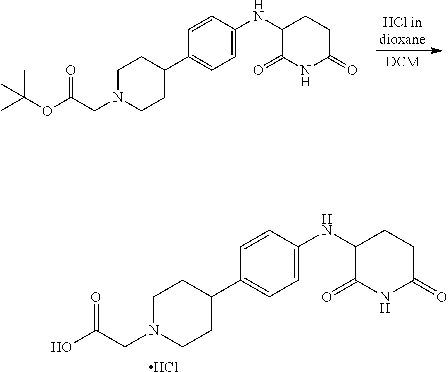

tert-Butyl 2-[4-[4-[(2,6-dioxo-3-piperidyl)amino]phenyl]-1-piperidyl]acetate (228 mg, 568 μmol) was dissolved in dichloromethane (2 mL) and 4M hydrochloric acid in 1,4 Dioxane (8 mmol, 2 mL) was added. The resulting mixture was stirred at room temperature for 4 h. The reaction mixture was concentrated under reduced pressure and the residue was triturated with diethyl ether and then filtered to afford 2-[4-[4-[(2,6-dioxo-3-piperidyl)amino]phenyl]-1-piperidyl]acetic acid hydrochloride (210 mg, 428 μmol) as grey solid. LCMS m/z: 345 (M+H⁺).

The following intermediates were synthesized from the appropriate starting materials using the above General procedure E for 2-[4-[4-[(2,6-dioxo-3-piperidyl)amino]phenyl]-1-piperidyl]acetic acid hydrochloride synthesis

| | | | LC |
|---|---|---|---|
| | | | MS |
| | | | (ESI+) |
| Starting material | Intermediate | Yield | m/z |
| 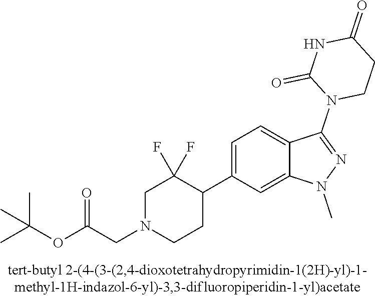 [SMILES: Cn1nc(N2CCC(=O)NC2=O)c2ccc(C3CCN(CC(=O)OC(C)(C)C)CC3(F)F)cc21] | 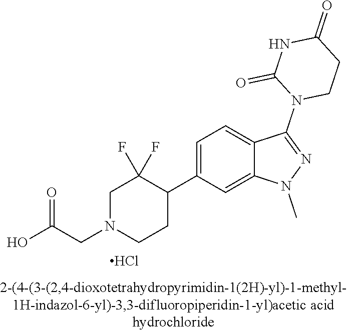 [SMILES: Cn1nc(N2CCC(=O)NC2=O)c2ccc(C3CCN(CC(=O)O)CC3(F)F)cc21 ; Cl] | 90% | |
| 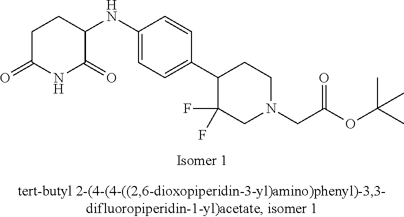 [SMILES: CC(C)(C)OC(=O)CN1CCC(c2ccc(NC3CCC(=O)NC3=O)cc2)C(F)(F)C1] | 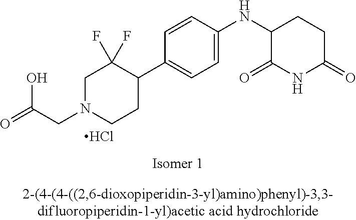 [SMILES: O=C(O)CN1CCC(c2ccc(NC3CCC(=O)NC3=O)cc2)C(F)(F)C1 ; Cl] | >98% | 382.2 (M + H) |
| 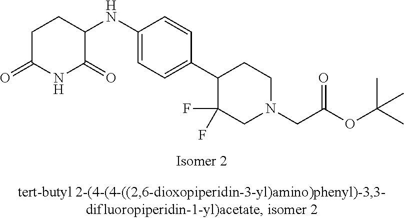 [SMILES: CC(C)(C)OC(=O)CN1CCC(c2ccc(NC3CCC(=O)NC3=O)cc2)C(F)(F)C1] | 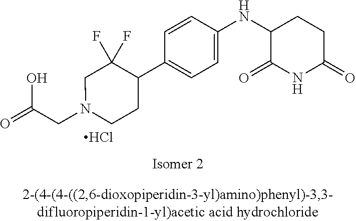 [SMILES: O=C(O)CN1CCC(c2ccc(NC3CCC(=O)NC3=O)cc2)C(F)(F)C1 ; Cl] | >98% | 382.2 (M + H) |

### Synthesis of Intermediate: Ethyl 2-amino-2-(6,7-dihydro-5H-pyrrolo[1,2-c]imidazol-1-yl)acetate dihydrochloride

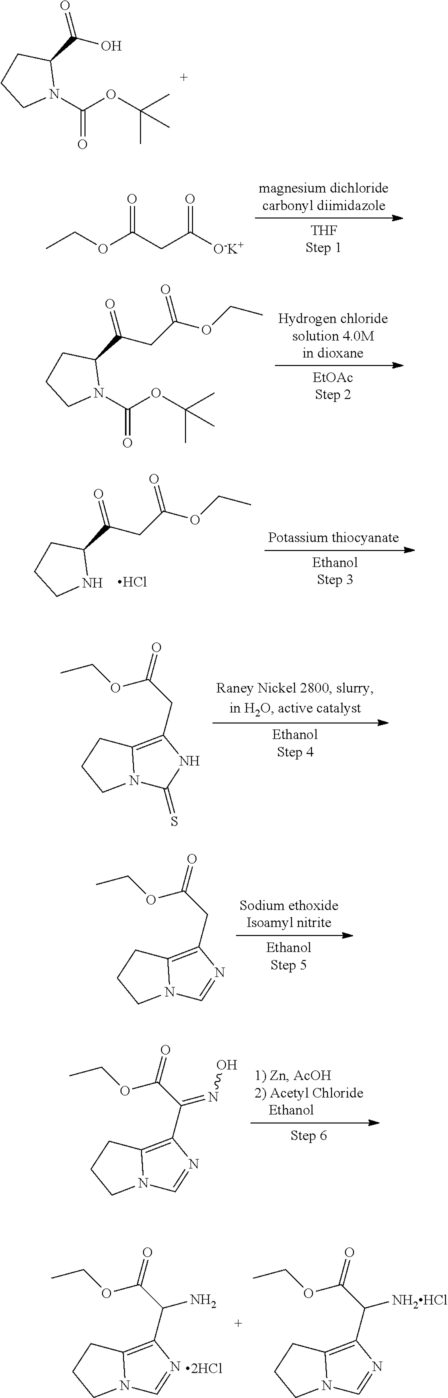

### Step 1: tert-butyl (S)-2-(3-ethoxy-3-oxopropanoyl)pyrrolidine-1-carboxylate

Potassium ethyl malonate (85.8 g, 504 mmol) was added to a stirred solution of magnesium chloride (31.0 g, 325 mmol) in THF (1400 mL) under argon atmosphere and the reaction mixture was heated at 50° C. for 6 h. In a separate flask, (2S)-1-tert-butoxycarbonylpyrrolidine-2-carboxylic acid (70.0 g, 325 mmol) was dissolved in THF (1400 mL), cooled at 0° C. and carbonyl diimidazole (81.7 g, 504 mmol) was added portion-wise over a period of 20 minutes. The reaction mixture was warmed to room temperature and stirred for another 2 h. The heated reaction mixture was cooled to room temperature and the activated ester solution was added drop-wise over a period of 20 min and the reaction mixture was stirred for 20 h. After completion of the reaction, the solvent was removed by distillation under reduced pressure to obtain thick white semisolid. This residue was dissolved in half saturated potassium bisulphate solution and extracted twice with ethyl acetate. The combined organic layers were washed with saturated NaHCO₃ solution, dried over sodium sulphate, filtered and concentrated to afford a crude light brown clear oil. This oil was purified by silica gel chromatography (10% Ethyl acetate:Pet ether) to afford tert-butyl (S)-2-(3-ethoxy-3-oxopropanoyl)pyrrolidine-1-carboxylate (76 g, 213 mmol, 66% yield) as a colorless clear oil. LCMS: (ESI−) (m/z: 284.0 [M−1]).

### Step 2: ethyl 3-oxo-3-[(2S)-pyrrolidin-2-yl]propanoate hydrochloride

tert-Butyl (2S)-2-(3-ethoxy-3-oxo-propanoyl)pyrrolidine-1-carboxylate (67.4 g, 236 mmol) was dissolved in ethyl acetate (70 mL) in a 2 L RBF. Hydrogen chloride (4 M in dioxane, 207 mL) was added. The reaction was stirred at rt overnight, then concentrated. The residual oil was stirred with MTBE (200 mL) overnight. The MTBE supernatant was decanted, the MTBE supernatant was discarded and the residue was concentrated under reduced pressure to give ethyl 3-oxo-3-[(2S)-pyrrolidin-2-yl]propanoate hydrochloride (52.4 g, 228 mmol, 97%) as a brown oil. LCMS (m/z: 186.1 [M+H]⁺)

### Step 3: ethyl 2-(3-thioxo-2,5,6,7-tetrahydropyrrolo[1,2-c]imidazol-1-yl)acetate

Potassium thiocyanate (23.3 g, 240 mmol, 12.3 mL) was added to a solution of ethyl 3-oxo-3-[(2S)-pyrrolidin-2-yl]propanoate hydrochloride (50.6 g, 228.26 mmol) in ethanol (250 mL). The heterogeneous mixture was stirred at 80° C. for 2 h, then was cooled and concentrated. The residue was dissolved in ethyl acetate (500 mL) and water (500 mL). Saturated aqueous NaHCO₃ (500 mL) was added to the mixture and extracted with ethyl acetate (3×500 mL). The combined organic layers were concentrated then mixed with 500 mL MTBE and stirred at rt overnight. The precipitate formed was collected by filtration to yield ethyl 2-(3-thioxo-2,5,6,7-tetrahydropyrrolo[1,2-c]imidazol-1-yl)acetate (47.1 g, 208 mmol, 91% yield) as a yellow solid. LCMS (m/z: 227.1 [M+H])

### Step 4: Ethyl 2-(6,7-dihydro-5H-pyrrolo[1,2-c]imidazol-1-yl)-2-oxo-acetate

To a 3-neck 2 L RBF was charged Raney Nickel®2800, slurry, in H2O, active catalyst (51.00 g, 595.27 mmol) and rinsed with EtOH (100 mL). The ethanol layer was discarded. A slurry of ethyl 2-(3-thioxo-2,5,6,7-tetrahydropyrrolo[1,2-c]imidazol-1-yl)acetate (51 g, 225.37 mmol) in EtOH (400 mL) was added. The mixture was refluxed at 80° C. for 2 h. The reaction mixture was cooled and filtered through a pad of Celite and washed with EtOH. The filtrate was concentrated to yield ethyl 2-(6,7-dihydro-5H-pyrrolo[1,2-c]imidazol-1-yl)-2-oxo-acetate as a brown oil (45.6 g, 235 mmol, quantitative yield). LCMS (ESI+): m/z: 194.9 [M+1]

### Step 5: Ethyl (2Z)-2-(6,7-dihydro-5H-pyrrolo[1,2-c]imidazol-1-yl)-2-hydroxyimino-acetate

To a solution of ethyl 2-(6,7-dihydro-5H-pyrrolo[1,2-c]imidazol-1-yl)acetate (20 g, 102.97 mmol) in Ethanol (100 mL) was added dropwise Sodium ethoxide, 21% w/w in ethanol (21.02 g, 308.91 mmol, 24.22 mL) at 0° C. The reaction mixture was stirred for 30 minutes. The reaction mixture was cooled to 0° C., followed by the drop wise addition of Isoamyl nitrite (41 mL, 309 mmol) at this temperature and after 3 hr, additional Isoamyl nitrite (27 mL, 206 mmol) was added to the reaction mixture. The reaction mixture was allowed slowly to reach room temperature and stirred under nitrogen atmosphere. The reaction mixture was diluted with ethanol (80 ml). The reaction mixture was neutralized by dropwise addition of 4M Hydrochloric acid in Methanol at 0° C., until a pH of 6 was obtained, using pH paper. The mixture turned to a light yellow colored suspension. The solid was filtered under suction, washed with ethanol (twice) and the filtrate were concentrated under reduced pressure. The crude material was diluted with water and extracted twice with dichloromethane and the combined organic layers were concentrated to afford ethyl 2-(6,7-dihydro-5H-pyrrolo[1,2-c]imidazol-1-yl)-2-hydroxyimino-acetate (17.2 g, 67.16 mmol, 65.22% yield) as a brown gum, which was used in the next step without further purification. LCMS (ESI+) m/z 224.0 (M+H).

### Step 6: ethyl 2-amino-2-(6,7-dihydro-5H-pyrrolo[1,2-c]imidazol-1-yl)acetate hydrochloride and ethyl 2-amino-2-(6,7-dihydro-5H-pyrrolo[1,2-c]imidazol-1-yl)acetate dihydrochloride

Ethyl 2-(6,7-dihydro-5H-pyrrolo[1,2-c]imidazol-1-yl)-2-hydroxyimino-acetate (30.40 g, 136.18 mmol) was dissolved in acetic acid (300 mL) in a 2 L 3-neck round-bottom flask with mechanic stir under nitrogen. Zinc (26.72 g, 408.55 mmol, 3.74 mL) was added. The reaction mixture was heated at 50° C. overnight. The reaction mixture was filtered through Celite and washed with additional acetic acid. The solvent was removed under reduced pressure to afford a brown crude residue. In a separate reaction vessel, Ethanol (125 mL) was cooled to 0° C. in a 3-neck 500 mL round-bottom flask. Acetyl chloride (28.14 g, 358.43 mmol, 21.81 mL) was added dropwise while keeping the internal temperature below 10° C. The solution was stirred for 15 minutes. The crude residue was added the solution. The reaction was stirred at room temperature for 16 hours. Then the mixture was heated at 50° C. for 24 hours. The reaction mixture was concentrated under reduced pressure. The crude was dissolved in 150 mL acetonitrile and 150 mL H₂O was added. White precipitate formed and the mixture was filtered. The filter cake to afford ethyl 2-amino-2-(6,7-dihydro-5H-pyrrolo[1,2-c]imidazol-1-yl)acetate hydrochloride (2.77 g, 11.3 mmol, 8.3% yield) as a white solid. ¹H NMR (400 MHz, DMSO-d₆) δ 8.54 (s, 3H), 7.65 (s, 1H), 5.16 (s, 1H), 4.57-4.10 (m, 2H), 4.01 (t, J=7.3 Hz, 2H), 2.75 (h, J=8.4 Hz, 2H), 2.55 (q, J=7.1 Hz, 2H), 1.20 (t, J=7.1 Hz, 3H). LCMS (ESI+): m/z 210.1 [M+H]. The mother liquor was concentrated and azeotroped with IPA (3×) to yield ethyl 2-amino-2-(6,7-dihydro-5H-pyrrolo[1,2-c]imidazol-1-yl)acetate dihydrochloride form of the desired product (32.7 g, 116.4 mmol, 86% yield) as a brown solid. LCMS (ESI+): m/z 210.1 [M+H]. ¹H NMR (400 MHz, DMSO-d₆) δ 8.93 (s, 4H), 8.56 (s, 1H), 5.46 (s, 1H), 4.45-4.05 (m, 5H), 3.06-2.69 (m, 3H), 2.58 (q, J=7.3 Hz, 2H), 1.23 (t, J=7.1 Hz, 4H).

### Synthesis of Intermediate: Ethyl 2-(6,7-dihydro-5H-pyrrolo[1,2-c]imidazol-1-yl)-2-(4-fluoro-6-iodo-1-oxo-isoindolin-2-yl)acetate

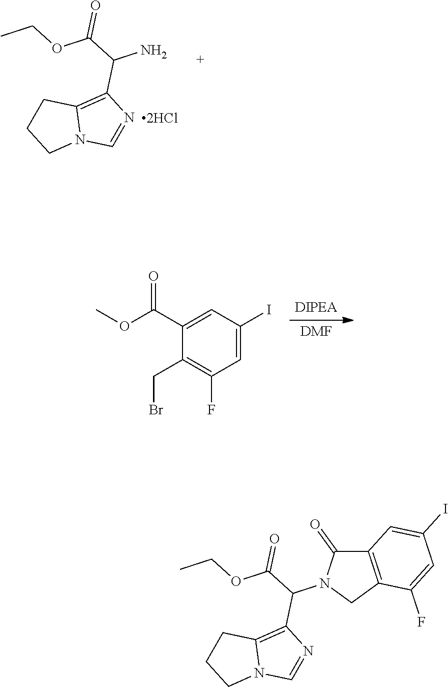

Ethyl 2-amino-2-(6,7-dihydro-5H-pyrrolo[1,2-c]imidazol-1-yl)acetate dihydrochloride (4.02 g, 12.62 mmol) was dissolved in DMF (36 mL). Methyl 2-(bromomethyl)-3-fluoro-5-iodo-benzoate (3.81 g, 10.22 mmol) was added, followed by N-ethyl-N-isopropyl-propan-2-amine (6.52 g, 50.47 mmol, 8.79 mL). The reaction mixture was stirred at rt for 16 hours. The reaction mixture was stirred at 45° C. The reaction mixture was diluted with ethyl acetate and washed with saturated aqueous sodium bicarbonate and brine. The organic layer was dried with sodium sulfate and concentrated to afford a brown oil, which was purified by flash column chromatography on silica gel (0-10% methanol in dichloromethane) to yield ethyl 2-(6,7-dihydro-5H-pyrrolo[1,2-c]imidazol-1-yl)-2-(4-fluoro-6-iodo-1-oxo-isoindolin-2-yl)acetate (2.03 g, 4.33 mmol, 34.29% yield) as a yellow solid. LCMS (ESI+): 470.2 (M+H), ¹H NMR (400 MHz, DMSO-d₆) δ 8.04-7.82 (m, 2H), 7.56 (s, 1H), 5.85 (s, 1H), 4.60 (d, J=18.0 Hz, 1H), 4.31-4.06 (m, 3H), 3.98 (q, J=6.9 Hz, 2H), 2.92-2.71 (m, 1H), 2.69-2.53 (m, 3H), 1.18 (t, J=7.1 Hz, 3H).

### Synthesis of Intermediate: Ethyl 2-(6-bromo-4-fluoro-1-oxo-isoindolin-2-yl)-2-(6,7-dihydro-5H-pyrrolo[1,2-c]imidazol-1-yl)acetate

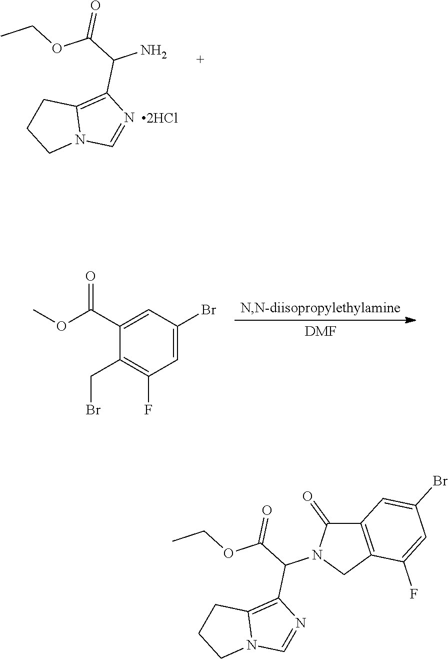

Ethyl 2-amino-2-(6,7-dihydro-5H-pyrrolo[1,2-c]imidazol-1-yl)acetate dihydrochloride (6.2 g, 21.97 mmol)) was dissolved in DMF (55 mL). Methyl 5-bromo-2-(bromomethyl)-3-fluoro-benzoate (5.73 g, 17.58 mmol) was added, followed by N-ethyl-N-isopropyl-propan-2-amine (11.36 g, 87.89 mmol, 15.31 mL). Reaction mixture was stirred at room temperature overnight. The reaction mixture was diluted with ethyl acetate and washed with saturated sodium bicarbonate and brine (2×). The organic layer was dried and concentrated to give a brown oil, which was purified by flash column chromatography on silica gel (0-10% methanol in dichloromethane) to provide ethyl 2-(6-bromo-4-fluoro-1-oxo-isoindolin-2-yl)-2-(6,7-dihydro-5H-pyrrolo[1,2-c]imidazol-1-yl)acetate (3.2 g, 7.58 mmol, 34.49% yield) as a yellow solid. LCMS (ESI+): 422.0/424.0 (M+H, Br pattern)

### Example 1. Synthesis of 5-[2-[7-Fluoro-3-oxo-2-[(1RS)-1-(6,7-dihydro-5H-pyrrolo[1,2-c]imidazol-1-yl)-2-oxo-2-(thiazol-2-ylamino)ethyl]isoindolin-5-yl]ethynyl]-N-[1-[2-[4-[4-[[(3RS)-2,6-dioxo-3-piperidyl]amino]phenyl]-1-piperidyl]acetyl]-4-piperidyl]pyridine-2-carboxamide

### Step 1: tert-Butyl 4-[4-[[(3RS)-2,6-dioxo-3-piperidyl]amino]phenyl]piperidine-1-carboxylate

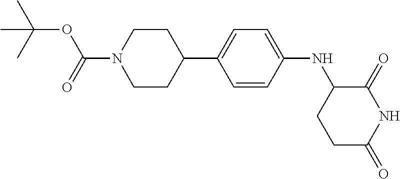 [SMILES: CC(C)(C)OC(=O)N1CCC(c2ccc(NC3CCC(=O)NC3=O)cc2)CC1]

tert-Butyl 4-(4-aminophenyl)piperidine-1-carboxylate (CAS 170011-57-1) (798 mg, 2.89 mmol) was dissolved in 10 ml of acetonitrile. Sodium bicarbonate (485 mg, 5.77 mmol, 2 equiv.) was added followed by 3-bromopiperidine-2,6-dione (CAS 62595-74-8) (610 mg, 3.18 mmol, 1.1 equiv.). The reaction mixture was stirred at 90° C. for 16 hours. The reaction mixture was cooled to room temperature, adsorbed on Isolute® and purified by flash chromatography on a silica gel column eluting with an ethyl acetate:heptane 30:70 to 100:0 gradient. The desired tert-butyl 4-[4-[[(3RS)-2,6-dioxo-3-piperidyl]amino]phenyl]piperidine-1-carboxylate (850 mg, 76% yield) was obtained as an off-white solid, MS: m/e=359.4 (([M-tBu+H]⁺).

### Step 2: (3RS)-3-[4-(4-Piperidyl)anilino]piperidine-2,6-dione hydrochloride

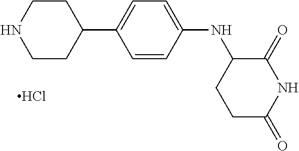 [SMILES: O=C1CCC(Nc2ccc(C3CCNCC3)cc2)C(=O)N1 ; Cl]

tert-Butyl 4-[4-[[(3RS)-2,6-dioxo-3-piperidyl]amino]phenyl]piperidine-1-carboxylate (850 mg, 2.19 mmol) and hydrochloric acid (4 M in dioxane) (5.48 ml, 21.9 mmol, 10 equiv.) were combined with 10 ml of methanol at 0-5° C. in an ice bath. The reaction mixture was stirred at room temperature for 18 hours. The reaction mixture was concentrated to dryness and used without further purification. The desired (3RS)-3-[4-(4-piperidyl)anilino]piperidine-2,6-dione hydrochloride (818 mg, quantitative, purity=87%) was obtained as an off-white solid, MS: m/e=286.1 ([M+H]⁺).

### Step 3: tert-Butyl 2-[4-[4-[[(3RS)-2,6-dioxo-3-piperidyl]amino]phenyl]-1-piperidyl]acetate

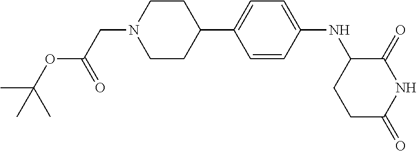 [SMILES: CC(C)(C)OC(=O)CN1CCC(c2ccc(NC3CCC(=O)NC3=O)cc2)CC1]

A mixture of (3RS)-3-[4-(4-piperidyl)anilino]piperidine-2,6-dione hydrochloride (200 mg, 0.618 mmol), tert-butyl 2-bromoacetate (CAS 5292-43-3) (157 mg, 0.119 ml, 0.803 mmol, 1.3 equiv.) and Hunig's base (399 mg, 0.539 ml, 3.09 mmol, 5 equiv.) in 4.0 ml of N,N-Dimethylformamide was stirred at room temperature for 2 hours. The reaction mixture was extracted with ethyl acetate and water. The aqueous layer was back-extracted with ethyl acetate. The organic layers were combined, dried over sodium sulfate, filtered and concentrated to dryness. The crude product was purified by flash chromatography on a silica gel column eluting with an ethyl acetate:heptane 50:50 to 100:0 gradient. The desired tert-butyl 2-[4-[4-[[(3RS)-2,6-dioxo-3-piperidyl]amino]phenyl]-1-piperidyl]acetate (164 mg, 66% yield) was obtained as a white solid, MS: m/e=402.2 ([M+H]⁺).

### Step 4: 2-[4-[4-[[(3RS)-2,6-Dioxo-3-piperidyl]amino]phenyl]-1-piperidyl]acetic acid trifluoroacetate

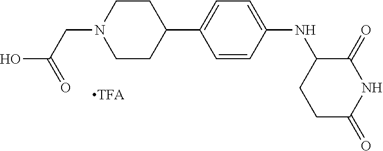 [SMILES: O=C(O)CN1CCC(c2ccc(NC3CCC(=O)NC3=O)cc2)CC1]

tert-Butyl 2-[4-[4-[[(3RS)-2,6-dioxo-3-piperidyl]amino]phenyl]-1-piperidyl]acetate (164 mg, 0.408 mmol) was combined with 3.0 ml of dichloromethane. Trifluoroacetic acid (1.48 g, 1 ml, 13 mmol, 31.8 equiv.) was added at 0-5° C. The reaction mixture was stirred at room temperature for 6 hours. The reaction mixture was concentrated in vacuo, dried under high vacuum and used without further purification. The desired 2-[4-[4-[[(3RS)-2,6-dioxo-3-piperidyl]amino]phenyl]-1-piperidyl]acetic acid trifluoroacetate (quantitative yield) was obtained as a light blue foam, MS: m/e=346.2 ([M+H]⁺).

### Example 1B. 2-(6,7-dihydro-5H-pyrrolo[1,2-c]imidazol-1-yl)-2-(6-(4-(4-(2-(4-(4-((2,6-dioxopiperidin-3-yl)amino)phenyl)piperidin-1-yl)-2-oxoethyl)piperazin-1-yl)phenyl)-4-fluoro-1-oxoisoindolin-2-yl)-N-(thiazol-2-yl)acetamide, Compound 10

### Step 1: Ethyl 2-(6,7-dihydro-5H-pyrrolo[1,2-c]imidazol-1-yl)-2-oxo-acetate

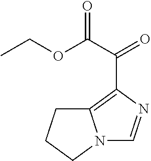

To a solution of ethyl 2-(6,7-dihydro-5H-pyrrolo[1,2-c]imidazol-1-yl)acetate (CAS 869113-97-3) (20.0 g, 102.97 mmol) dissolved in 200 ml of 1,4-dioxane was added selenium dioxide (22.85 g, 205.94 mmol, 2 equiv.). The reaction mixture was stirred for 5 hours at 80° C. The reaction mixture was concentrated under vacuum to give a residue. The crude product was purified by flash chromatography on a silica gel column eluting with petroleum ether:ethyl acetate 2:1 to ethyl acetate:ethanol 10:1 gradient to obtain the desired ethyl 2-(6,7-dihydro-5H-pyrrolo[1,2-c]imidazol-1-yl)-2-oxo-acetate (quantitative yield) as a light brown oil, MS: m/e=209.1 ([M+H]⁺)

### Step 2: Ethyl 2-(6,7-dihydro-5H-pyrrolo[1,2-c]imidazol-1-yl)-2-hydroxyimino-acetate

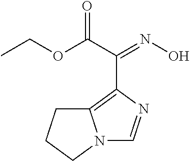 [SMILES: CCOC(=O)/C(=N/O)c1ncn2c1CCC2]

To a solution of ethyl 2-(6,7-dihydro-5H-pyrrolo[1,2-c]imidazol-1-yl)-2-oxo-acetate (17.5 g, 84.05 mmol) dissolved in 145 ml of ethanol was added hydroxylamine hydrochloride (6.42 g, 92.45 mmol, 1.1 equiv.) and sodium acetate (13.79 g, 168.1 mmol, 2 equiv.) at room temperature. The reaction mixture was stirred at 80° C. for 3.5 hours. The reaction mixture was concentrated and extracted with water and five times with a mixture of ethanol/THF/ethyl acetate 1:1:8. The organic layers were concentrated to dryness. The desired ethyl 2-(6,7-dihydro-5H-pyrrolo[1,2-c]imidazol-1-yl)-2-hydroxyimino-acetate (15 g, 80% yield) was obtained as a yellow solid, MS: m/e=224.1 ([M+H]⁺) and used directly in the next step.

### Step 3: Ethyl (2RS)-2-amino-2-(6,7-dihydro-5H-pyrrolo[1,2-c]imidazol-1-yl)acetate

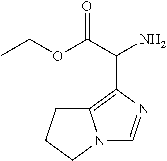

To a solution of ethyl 2-(6,7-dihydro-5H-pyrrolo[1,2-c]imidazol-1-yl)-2-hydroxyimino-acetate (15.0 g, 67.2 mmol) dissolved in 225 ml of ethanol and 120 ml of THF was added Pd/C (30.0 g, 67.2 mmol, 1 eq, 10%) at room temperature. The mixture was hydogenated with H₂ at 45° C. for 24 hours. The reaction mixture was filtered and the filtrate was concentrated under vacuum. The desired ethyl (2RS)-2-amino-2-(6,7-dihydro-5H-pyrrolo[1,2-c]imidazol-1-yl)acetate (quantitative yield) was obtained as a brown oil, MS: m/e=210.1 ([M+H]⁺) and used directly in the next step.

### Step 4: Ethyl (2RS)-2-amino-2-(6,7-dihydro-5H-pyrrolo[1,2-c]imidazol-1-yl)acetate hydrochloride

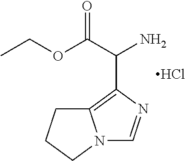

A solution of ethyl (2RS)-2-amino-2-(6,7-dihydro-5H-pyrrolo[1,2-c]imidazol-1-yl)acetate (15.0 g, 82.79 mmol) in HCl/EtOH (300 ml, 1200 mmol, 14.5 equiv., 2.5 mol/L) was stirred at 25° C. for 36 hours. The reaction mixture was concentrated under vacuum below 25° C. to give a residue as brown oil. 150 ml of acetonitrile were added to the residue and the precipitated yellow solid was collected and dried under vacuum below 25° C. to give the desired ethyl (2RS)-2-amino-2-(6,7-dihydro-5H-pyrrolo[1,2-c]imidazol-1-yl)acetate hydrochloride (quantitative yield) as yellow solid, MS: m/e=210.1 ([M+H]⁺).

### Step 5: Methyl 5-bromo-2-(bromomethyl)-3-fluoro-benzoate

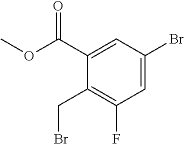

Methyl 5-bromo-3-fluoro-2-methylbenzoate (CAS 2090424-20-5) (5.91 g, 23.9 mmol) was dissolved in 100 ml trifluorotoluene and N-bromosuccinimide (4.26 g, 23.9 mmol, 1 equiv.) and AIBN (393 mg, 2.39 mmol, 0.1 equiv.) were added at room temperature. The mixture was stirred at 110° C. for 3 hours. The reaction mixture was cooled, extracted with water and two times with ethyl acetate. The organic layers were dried over sodium sulfate and concentrated to dryness. The crude product was purified by flash chromatography on a silica gel column eluting with an ethyl acetate:heptane 0:100 to 50:50 gradient to obtain the desired methyl 5-bromo-2-(bromomethyl)-3-fluoro-benzoate (7.29 g, 94% yield) as a light yellow liquid, MS: m/e=326.8 ([M+H]⁺).

### Step 6: Ethyl (2RS)-2-(6-bromo-4-fluoro-1-oxo-isoindolin-2-yl)-2-(6,7-dihydro-5H-pyrrolo[1,2-c]imidazol-1-yl)acetate

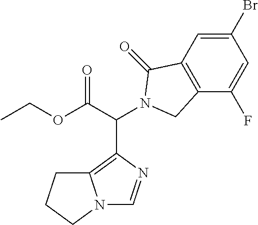 [SMILES: CCOC(=O)C(c1ncn2c1CCC2)N1Cc2c(F)cc(Br)cc2C1=O]

Ethyl (2RS)-2-amino-2-(6,7-dihydro-5H-pyrrolo[1,2-c]imidazol-1-yl)acetate hydrochloride (4.15 g, 16.9 mmol, 1 equiv.) was dissolved in 35 ml of N,N-Dimethylformamide. Methyl 5-bromo-2-(bromomethyl)-3-fluoro-benzoate (5.0 g, 15.3 mmol) and triethylamine (10.7 ml, 76.7 mmol, 5 equiv.) were added at room temperature. The mixture was stirred at 80° C. for 16 hours. The reaction mixture was extracted with water and two times with ethyl acetate. The organic layers were extracted with brine, dried over sodium sulfate and concentrated to dryness. The crude product was purified by flash chromatography on a silica gel column eluting with a dichloromethane:methanol 100:0 to 90:10 gradient to obtain the desired ethyl (2RS)-2-(6-bromo-4-fluoro-1-oxo-isoindolin-2-yl)-2-(6,7-dihydro-5H-pyrrolo[1,2-c]imidazol-1-yl)acetate (2.6 g, 40% yield) as a yellow solid, MS: m/e=422.1/424.1 ([M+H]⁺).

### Step 7: tert-Butyl 4-[4-[7-fluoro-3-oxo-2-[(1RS)-1-(6,7-dihydro-5H-pyrrolo[1,2-c]imidazol-1-yl)-2-ethoxy-2-oxo-ethyl]isoindolin-5-yl]phenyl]piperazine-1-carboxylate

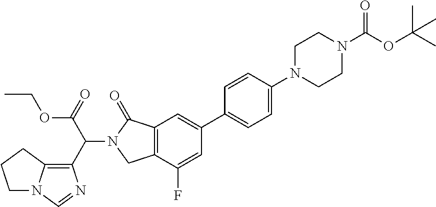 [SMILES: CCOC(=O)C(c1ncn2c1CCC2)N1Cc2c(F)cc(-c3ccc(N4CCN(C(=O)OC(C)(C)C)CC4)cc3)cc2C1=O]

Ethyl (2RS)-2-(6-bromo-4-fluoro-1-oxo-isoindolin-2-yl)-2-(6,7-dihydro-5H-pyrrolo[1,2-c]imidazol-1-yl)acetate (56 mg, 0.133 mmol) and (4-(4-(tert-butoxycarbonyl)piperazin-1-yl)phenyl)boronic acid (CAS 457613-78-4) (41 mg, 0.133 mmol, 1.0 equiv.) were dissolved in 1.0 ml of 1,2-dimethoxyethane and 2M aq. Na₂CO₃-solution (0.199 ml, 0.398 mmol, 3.0 equiv.). Tetrakis(triphenylphosphine)palladium (0) (15 mg, 0.0133 mmol, 0.1 equiv.) was added and the reaction mixture was stirred at 80° C. for 4 hours. The reaction mixture was cooled to room temperature and then extracted with ethyl acetate and saturated NaHCO₃-solution. The aqueous layer was back-extracted with ethyl acetate. The organic layers were washed with water and brine. The organic layers were combined, dried over sodium sulfate, filtered and concentrated to dryness. The crude product was purified by flash chromatography on a silica gel column eluting with an ethyl acetate:heptane 5:95 to 100:0 gradient. The desired tert-butyl 4-[4-[7-fluoro-3-oxo-2-[(1RS)-1-(6,7-dihydro-5H-pyrrolo[1,2-c]imidazol-1-yl)-2-ethoxy-2-oxo-ethyl]isoindolin-5-yl]phenyl]piperazine-1-carboxylate (48 mg, 60% yield) was obtained as a light brown oil, MS: m/e=604.4 ([M+H]⁺).

### Step 8: tert-Butyl 4-[4-[7-fluoro-3-oxo-2-[(1RS)-1-(6,7-dihydro-5H-pyrrolo[1,2-c]imidazol-1-yl)-2-oxo-2-(thiazol-2-ylamino)ethyl]isoindolin-5-yl]phenyl]piperazine-1-carboxylate

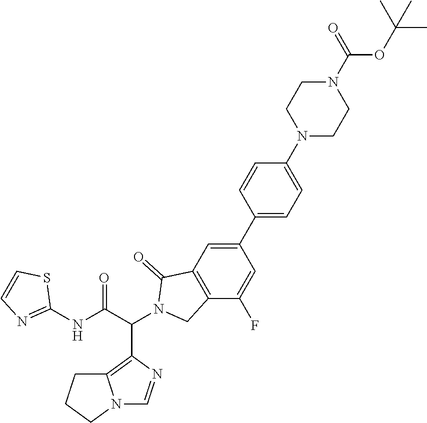 [SMILES: CC(C)(C)OC(=O)N1CCN(c2ccc(-c3cc(F)c4c(c3)C(=O)N(C(C(=O)Nc3nccs3)c3ncn5c3CCC5)C4)cc2)CC1]

tert-Butyl 4-[4-[7-fluoro-3-oxo-2-[(1RS)-1-(6,7-dihydro-5H-pyrrolo[1,2-c]imidazol-1-yl)-2-ethoxy-2-oxo-ethyl]isoindolin-5-yl]phenyl]piperazine-1-carboxylate (48 mg, 0.0795 mmol) was combined with 11 ml of ethanol to give a light yellow solution. LiOH (1M in water) (0.0954 ml, 0.0954 mmol, 1.2 equiv.) was added. The reaction mixture was stirred at room temperature for 2 hours. The reaction mixture was concentrated in vacuo. The residue was taken up in ethanol and concentrated in vacuo and then dissolved in 7.0 ml of N,N-Dimethylformamide. Thiazol-2-amine (9.55 mg, 0.0954 mmol, 1.2 equiv.) and Hunig's base (0.0694 ml, 0.398 mmol, 5 equiv.) were added followed by HATU (36.3 mg, 0.0954 mmol, 1.2 equiv.). The mixture was stirred at room temperature for 1 hour. The reaction mixture was extracted with ethyl acetate and saturated NaHCO₃-solution. The aqueous layer was back-extracted with ethyl acetate. The organic layers were washed with water and brine. The organic layers were combined, dried over sodium sulfate, filtered and concentrated to dryness. The crude product was purified by flash chromatography on a silica gel column eluting with a dichloromethane:methanol 100:0 to 90:10 gradient to obtain the desired tert-butyl 4-[4-[7-fluoro-3-oxo-2-[(1RS)-1-(6,7-dihydro-5H-pyrrolo[1,2-c]imidazol-1-yl)-2-oxo-2-(thiazol-2-ylamino)ethyl]isoindolin-5-yl]phenyl]piperazine-1-carboxylate (22 mg, 42% yield) as a yellow oil, MS: m/e=658.3 ([M+H]⁺).

### Step 9: (2RS)-2-(6,7-Dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide

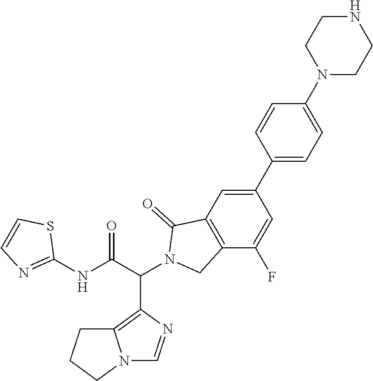 [SMILES: O=C(Nc1nccs1)C(c1ncn2c1CCC2)N1Cc2c(F)cc(-c3ccc(N4CCNCC4)cc3)cc2C1=O]

tert-Butyl 4-[4-[7-fluoro-3-oxo-2-[(1RS)-1-(6,7-dihydro-5H-pyrrolo[1,2-c]imidazol-1-yl)-2-oxo-2-(thiazol-2-ylamino)ethyl]isoindolin-5-yl]phenyl]piperazine-1-carboxylate (26 mg, 0.0395 mmol) was dissolved in 0.5 ml of dichloromethane and 0.25 ml of methanol. HCl (4 M in dioxane) (0.099 ml, 0.395 mmol, 10 equiv.) was added at room temperature and the reaction mixture was stirred at room temperature for 16 hours. The reaction mixture was extracted with saturated NaHCO₃-solution and twice with dichloromethane. The organic layers were combined, dried over sodium sulfate, filtered and concentrated to dryness. The desired (2RS)-2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide (22 mg, 99.8% yield) was obtained as a light yellow oil, MS: m/e=558.2 ([M+H]⁺).

### Step 10: tert-Butyl 2-[4-[4-[7-fluoro-3-oxo-2-[(1RS)-1-(6,7-dihydro-5H-pyrrolo[1,2-c]imidazol-1-yl)-2-oxo-2-(thiazol-2-ylamino)ethyl]isoindolin-5-yl]phenyl]piperazin-1-yl]acetate

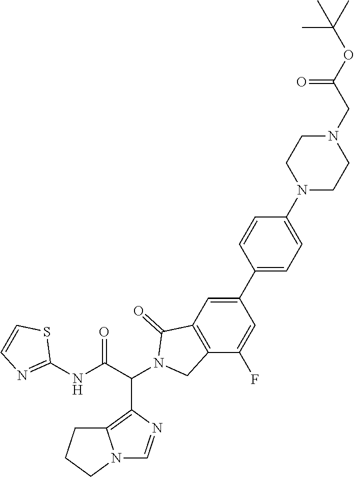 [SMILES: CC(C)(C)OC(=O)CN1CCN(c2ccc(-c3cc(F)c4c(c3)C(=O)N(C(C(=O)Nc3nccs3)c3ncn5c3CCC5)C4)cc2)CC1]

The title compound was obtained as a yellow oil, using chemistry similar to that described in Example 1, step 3 starting from (2RS)-2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide and tert-butyl 2-bromoacetate (CAS 5292-43-3).

### Example 2. 2-(6,7-dihydro-5H-pyrrolo[1,2-c]imidazol-1-yl)-2-[6-[4-[2-[2-[1-[4-[[(3S)-2,6-dioxo-3-piperidyl]amino]-2-fluoro-phenyl]-4-hydroxy-4-piperidyl]acetyl]-2,6-diazaspiro[3.3]heptan-6-yl]phenyl]-4-fluoro-1-oxo-isoindolin-2-yl]-N-thiazol-2-yl-acetamide (Compound 1)

### Step 1: 1-(2-fluoro-4-nitro-phenyl)piperidin-4-one

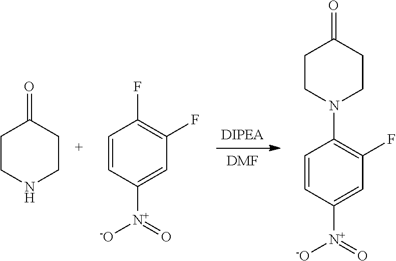

To a solution of piperidin-4-one (15.0 g, 151.31 mmol), 1,2-difluoro-4-nitro-benzene (24.07 g, 151.31 mmol, 16.72 mL) in N,N-dimethylformamide (30 mL) was added N,N-diisopropylethylamine (78.22 g, 605.26 mmol, 105.42 mL) and heated at 110° C. for 14 h. The reaction mixture was diluted with ethyl acetate (500 mL) and washed with cold water (150 mL). The organic layer was washed with a brine solution (150 mL), dried over sodium sulfate and concentrated under reduced pressure. The residue was purified by column chromatography on silica gel (40% ethyl acetate in petroleum ether) to afford 1-(2-fluoro-4-nitro-phenyl)piperidin-4-one (21 g, 77.93 mmol, 51.50% yield) as a brown solid. LCMS, m/z: 238.9 [M+H]⁺

### Step 2: Synthesis of tert-butyl 2-[1-(4-amino-2-fluoro-phenyl)-4-hydroxy-4-piperidyl]acetate

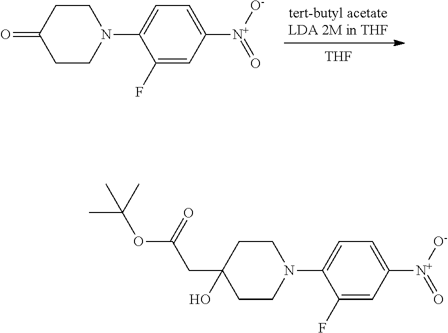 [SMILES: O=C1CCN(c2ccc([N+](=O)[O-])cc2F)CC1 ; CC(C)(C)OC(=O)CC1(O)CCN(c2ccc([N+](=O)[O-])cc2F)CC1 ; "THF"]

Lithium diisopropylamide (2 M, 12.59 mL) was added dropwise to a stirred solution of tert-butyl acetate (1.76 g, 15.11 mmol, 2.03 mL) in tetrahydrofuran (25 mL) at −78° C. The reaction mixture was stirred at −78° C. for 45 minutes. 1-(2-fluoro-4-nitro-phenyl)piperidin-4-one (3 g, 12.59 mmol) dissolved in tetrahydrofuran (15 mL) was added to it at −78° C. The reaction mixture was stirred at −78° C. for 2 h. The reaction mixture was quenched with saturated ammonium chloride solution and the mixture was extracted with ethyl acetate. The organic layer was dried over sodium sulfate and concentrated under reduced pressure. The residue was purified by silica gel (100-200 mesh) column chromatography (30% to 40% Ethyl acetate in Petroleum ether as eluent) to afford tert-butyl 2-[1-(2-fluoro-4-nitro-phenyl)-4-hydroxy-4-piperidyl]acetate (2.7 g, 7.01 mmol, 55.66% yield) as light yellow sticky solid. LCMS: 355.1 (M+H)⁺

### Step 3: tert-butyl 2-[1-(4-amino-2-fluoro-phenyl)-4-hydroxy-4-piperidyl]acetate

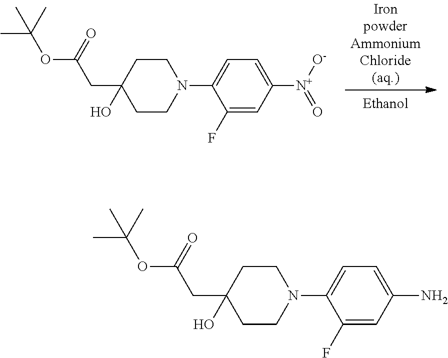 [SMILES: CC(C)(C)OC(=O)CC1(O)CCN(c2ccc([N+](=O)[O-])cc2F)CC1 ; "Ethanol" ; CC(C)(C)OC(=O)CC1(O)CCN(c2ccc(N)cc2F)CC1]

To a solution of tert-butyl 2-[1-(2-fluoro-4-nitro-phenyl)-4-hydroxy-4-piperidyl]acetate (1.5 g, 4.23 mmol) in Ethanol (10 mL) and water (2 mL) were added iron powder (1.18 g, 21.16 mmol, 150.37 μL) and ammonium chloride (679.26 mg, 12.70 mmol, 443.96 μL). The reaction was stirred at 70° C. for 4 h. The reaction mixture was filtered through celite and the filter cake was washed with ethyl acetate (60 mL). The filtrate was washed with water (20 mL), aqueous sodium bicarbonate (20 mL) and brine (20 mL). The organic layer was dried over sodium sulfate and concentrated under reduced pressure to get crude, which was purified by column chromatography on silica gel eluted with 70% ethyl acetate in petroleum ether to afford tert-butyl 2-[1-(4-amino-2-fluoro-phenyl)-4-hydroxy-4-piperidyl]acetate (1.2 g, 3.44 mmol, 81.28% yield) as a brown sticky solid. LCMS m/z: 325.1 [M+H], ¹HNMR (DMSO-d₆) 8.02-7.89 (m, 2H), 7.23-7.05 (m, 1H), 4.69 (s, 1H), 3.55-3.43 (m, 2H), 3.22-3.19 (m, 2H), 2.36 (s, 2H), 1.88-1.64 (m, 3H), 1.41 (s, 9H).

### Step 4: tert-butyl 2-[1-[4-[(2,6-dioxo-3-piperidyl)amino]-2-fluoro-phenyl]-4-hydroxy-4-piperidyl]acetate

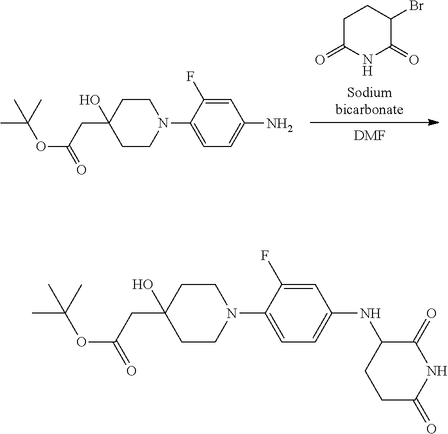

To a stirred solution of tert-butyl 2-[1-(4-amino-2-fluoro-phenyl)-4-hydroxy-4-piperidyl]acetate (1 g, 3.08 mmol) in N,N-dimethylformamide (10 mL) were added sodium bicarbonate (517.94 mg, 6.17 mmol) under nitrogen atmosphere in 25 ml seal tube. The vial was sealed and heated at 60° C. overnight. The reaction mixture was filtered through celite bed, washed 2 times with ethyl acetate and filtrate was concentrated under reduced pressure at 35° C. The crude residue was purified over silica column (100-200 mesh) eluting the compound in 65-70% ethyl acetate in petroleum ether. Pure fractions were evaporated under reduced pressure to afford the desired compound tert-butyl 2-[1-[4-[(2,6-dioxo-3-piperidyl)amino]-2-fluoro-phenyl]-4-hydroxy-4-piperidyl]acetate (760 mg, 1.67 mmol, 54.11% yield) as an off white solid. LCMS m/z: 436.0 [M+H], ¹H-NMR (DMSO-d₆): 10.79 (s, 1H), 6.87-6.80 (m, 1H), 6.52 (dd, J=13.6 Hz, 3.6 Hz, 1H), 6.41 (dd, J=3.7 Hz, 1.6 Hz, 1H), 4.89 (d, J=3.6 Hz, 1H), 4.45 (s, 1H), 4.30-4.19 (m, 1H), 2.90-2.80 (m, 4H), 2.78-2.51 (m, 3H), 2.49-2.41 (m, 1H), 2.13-2.01 (m, 2H), 1.95-1.63 (m, 4H), 1.42 (s, 9H).

### Step 5: tert-Butyl 2-[1-[4-[[(3S)-2,6-dioxo-3-piperidyl]amino]-2-fluoro-phenyl]-4-hydroxy-4-piperidyl]acetate

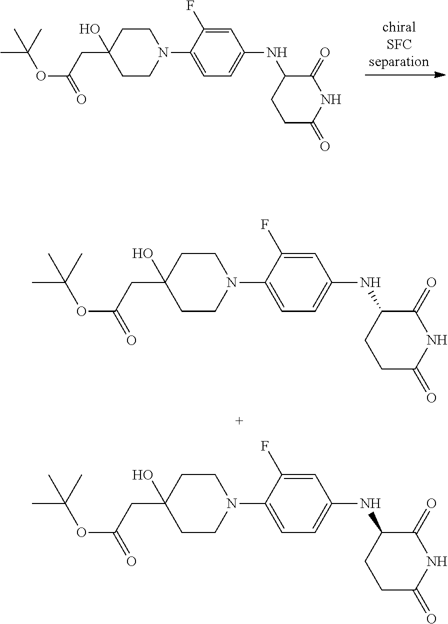 [SMILES: CC(C)(C)OC(=O)CC1(O)CCN(c2ccc(NC3CCC(=O)NC3=O)cc2F)CC1 ; CC(C)(C)OC(=O)CC1(O)CCN(c2ccc(N[C@H]3CCC(=O)NC3=O)cc2F)CC1 ; CC(C)(C)OC(=O)CC1(O)CCN(c2ccc(N[C@@H]3CCC(=O)NC3=O)cc2F)CC1]

The racemic mixture tert-butyl 2-[1-[4-[(2,6-dioxo-3-piperidyl)amino]-2-fluoro-phenyl]-4-hydroxy-4-piperidyl]acetate (2 g, 4.59 mmol) was resolved by chiral SFC. 2.0 g of sample was dissolved in 22.0 mL of acetonitrile.

SFC separation conditions: Column: LUX A1 [250*10 mm, 5-micron particle size]; Mobile phase: CO₂: Isopropanol (45:55); Flow rate: 12 g/min; Cycle time: 11.0 min; Back pressure: 100 bar UV collection, wavelength: 254 nm; Volume: 0.4 mL per injection

The first eluting set of fractions was evaporated under pressure to afford tert-butyl 2-[1-[4-[[(3S)-2,6-dioxo-3-piperidyl]amino]-2-fluoro-phenyl]-4-hydroxy-4-piperidyl]acetate (850 mg, 1.84 mmol, 40.04% yield) as an off white solid. LCMS m/z: 436.0 [M+H], LCMS (ESI+) m/z: 436.2 [M+H]+. 1H-NMR (400 MHz, DMSO-d₆): δ 10.77 (s, 1H), 6.83 (t, J=12.00 Hz, 1H), 6.49 (d, J=20.00 Hz, 1H), 6.41 (d, J=12.00 Hz, 1H), 5.77 (d, J=7.60 Hz, 1H), 4.44 (s, 1H), 4.29-4.12 (m, 1H), 2.91-2.79 (m, 5H), 2.74-2.70 (m, 1H), 2.34 (s, 2H), 2.16-2.02 (m, 1H), 1.89-1.69 (m, 3H), 1.65 (d, J=16.80 Hz, 2H), 1.42 (s, 9H), 99.18% ee by chiral SFC (Rt=2.33 min), Specific optical rotation: −46.2° [α]²⁰_{D}

The second eluting set of fractions was evaporated under pressure to afford tert-butyl 2-[1-[4-[[(3R)-2,6-dioxo-3-piperidyl]amino]-2-fluoro-phenyl]-4-hydroxy-4-piperidyl]acetate (530 mg, 1.17 mmol, 25.52% yield) as an off white solid. LCMS m/z: 436.0 [M+H], LCMS (ESI+) m/z: 436.0 [M+H]+. 1H-NMR (400 MHz, DMSO-d₆): δ 10.78 (s, 1H), 6.84 (t, J=13.20 Hz, 1H), 6.49 (d, J=20.00 Hz, 1H), 6.41 (d, J=12.40 Hz, 1H), 5.77 (d, J=10.40 Hz, 1H), 4.44 (s, 1H), 4.27-4.22 (m, 1H), 2.92-2.77 (m, 5H), 2.73-2.63 (m, 1H), 2.34 (s, 2H), 2.18-2.03 (m, 1H), 1.87-1.73 (m, 3H), 1.64 (d, J=18.00 Hz, 2H), 1.48 (s, 9H), 99.13% ee by chiral SFC (Rt=4.92 min), Specific optical rotation: +46.8° [α]²⁰_{D}

### Absolute Configuration Compound 1

The absolute configuration of Compound 1 was established by establishing the absolute configuration of intermediate tert-Butyl 2-[1-[4-[[(3S)-2,6-dioxo-3-piperidyl]amino]-2-fluoro-phenyl]-4-hydroxy-4-piperidyl]acetate by X-ray diffraction of a co-crystal with a cereblon protein construct. Stereochemical integrity of final Compound 1 was established by HPLC chiral chromatography. No racemization of the glutarimide chiral center was observed.

### Absolute Configuration Determination of Intermediate tert-Butyl 2-[1-[4-[[(3S)-2,6-dioxo-3-piperidyl]amino]-2-fluoro-phenyl]-4-hydroxy-4-piperidyl]acetate by X-ray crystallography

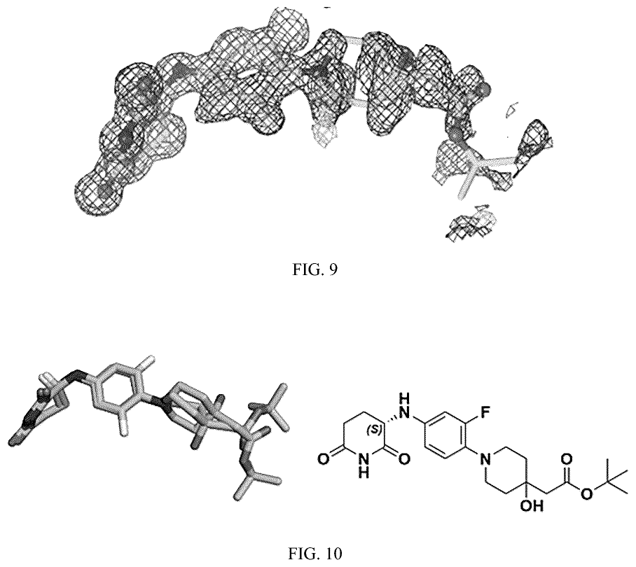

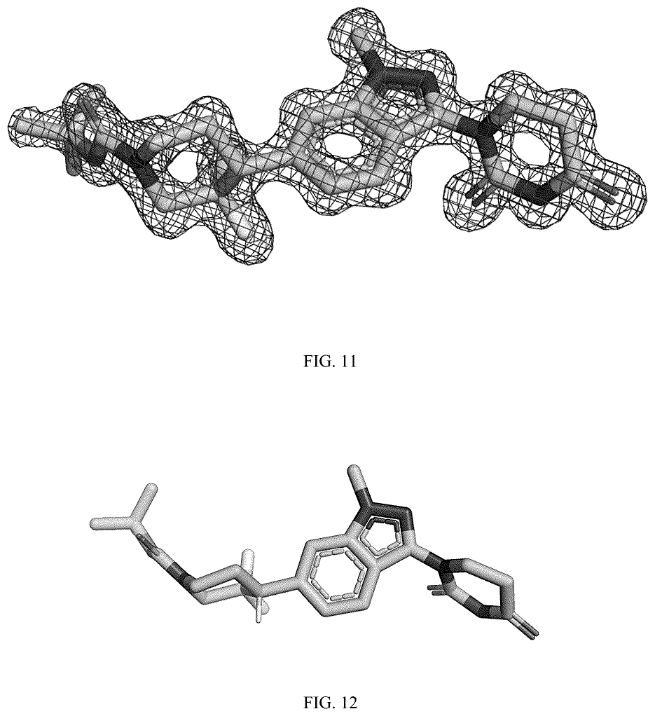

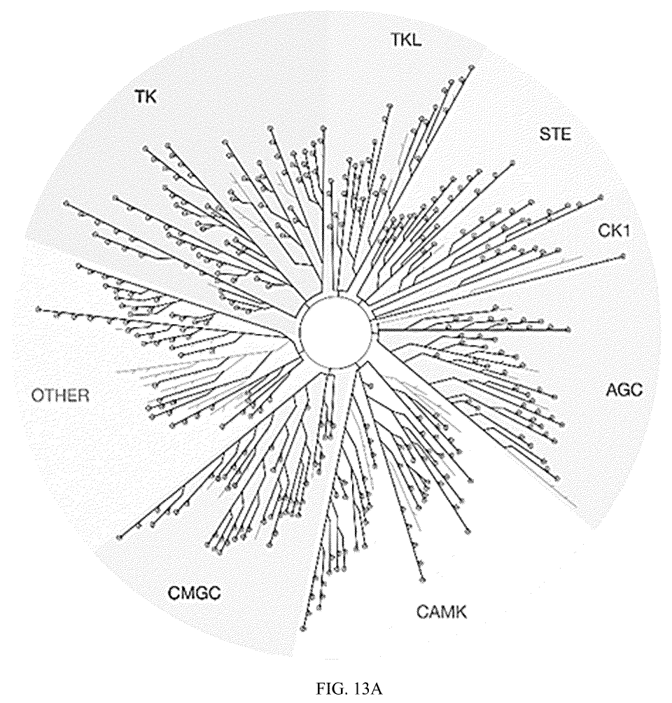

The absolute configuration of tert-Butyl 2-[1-[4-[[(3S)-2,6-dioxo-3-piperidyl]amino]-2-fluoro-phenyl]-4-hydroxy-4-piperidyl]acetate by X-ray crystallography was defined at ultra-high resolution via co-crystallisation with a Cereblon (CRBN) protein construct, followed by X-ray diffraction of both the protein and small molecule bound in situ. The structure resolution was 1 Å. The resulting structure with and without a density map is shown in FIG. 8 and FIG. 9.

### Step 6: 2-[1-[4-[[(3S)-2,6-dioxo-3-piperidyl]amino]-2-fluoro-phenyl]-4-hydroxy-4-piperidyl]acetic acid hydrochloride

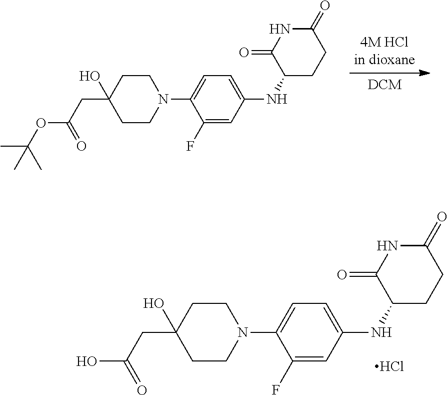

To a stirred solution of tert-butyl 2-[1-[4-[[(3S)-2,6-dioxo-3-piperidyl]amino]-2-fluoro-phenyl]-4-hydroxy-4-piperidyl]acetate (600 mg, 1.38 mmol) in dichloromethane (15 mL) at 0° C. was added hydrogen chloride (4M solution in 1,4-dioxane, 1.72 mL, 6.89 mmol) dropwise. The reaction mixture was stirred at room temperature for 6 h. The volatiles were removed by rotary evaporation. The residue was triturated twice with diethyl ether (2×10 mL). The solid residue was dried under rotary vacuum to afford 2-[1-[4-[[(3S)-2,6-dioxo-3-piperidyl]amino]-2-fluoro-phenyl]-4-hydroxy-4-piperidyl]acetic acid hydrochloride (610 mg, 1.09 mmol, 78.96% yield) as a grey solid. LCMS (ESI+) m/z: 380.0 [M+H]+, ¹H-NMR (400 MHz, DMSO-d₆): δ 12.03 (bs, 1H), 10.86 (s, 1H), 7.63 (s, 1H), 6.70 (d, J=15.20 Hz, 1H), 6.58 (dd, J=11.40, 6.80 Hz, 1H), 4.43 (dd, J=11.60, 4.40 Hz, 1H), 3.88-3.65 (m, 5H), 3.41-3.36 (m, 2H), 2.74-2.68 (m, 1H), 2.59-2.54 (m, 1H), 2.46 (s, 2H), 2.33 (bs, 2H), 2.10-2.08 (m, 1H), 1.94-1.88 (m, 2H).

### Step 7: 2-(6,7-dihydro-5H-pyrrolo[1,2-c]imidazol-1-yl)-2-[6-[4-[2-[2-[1-[4-[[(3S)-2,6-dioxo-3-piperidyl]amino]-2-fluoro-phenyl]-4-hydroxy-4-piperidyl]acetyl]-2,6-diazaspiro-[3.3]heptan-6-yl]phenyl]-4-fluoro-1-oxo-isoindolin-2-yl]-N-thiazol-2-yl-acetamide

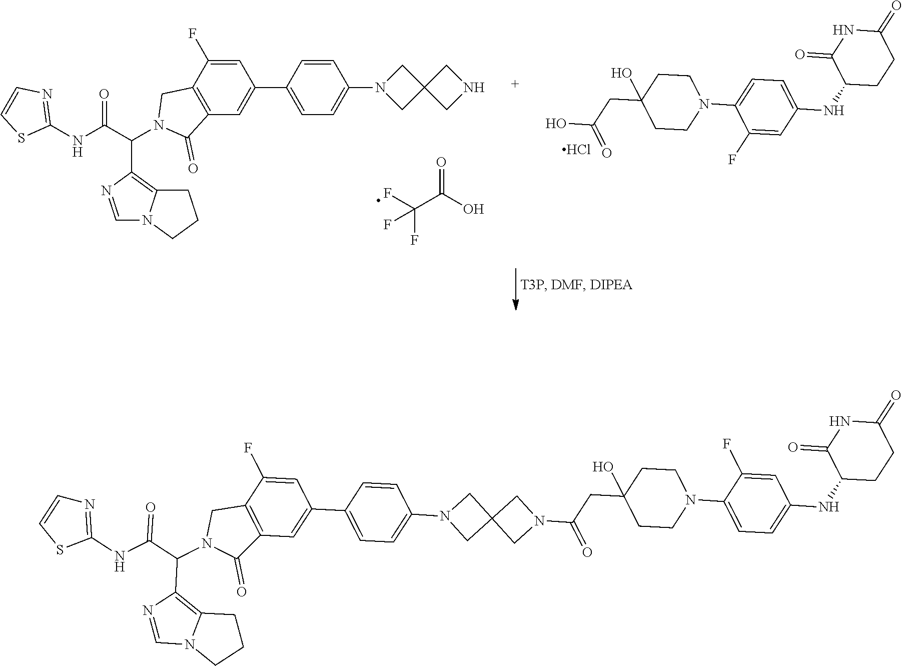 [SMILES: O=C(Nc1nccs1)C(c1ncn2c1CCC2)N1Cc2c(F)cc(-c3ccc(N4CC5(CNC5)C4)cc3)cc2C1=O ; O=C(O)C(F)(F)F ; O=C(O)CC1(O)CCN(c2ccc(N[C@H]3CCC(=O)NC3=O)cc2F)CC1 ; Cl ; O=C1CC[C@H](Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1]

2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide, trifluoroacetic acid salt (270 mg, 394.92 μmol) and 2-[1-[4-[[(3S)-2,6-dioxo-3-piperidyl]amino]-2-fluoro-phenyl]-4-hydroxy-4-piperidyl]acetic acid hydrochloride (197.07 mg, 473.91 μmol) were mixed in N,N-dimethylformamide (5 mL). N,N-diisopropylethylamine (357.29 mg, 2.76 mmol, 481.52 μL) was added to the reaction mixture at 0° C. Propylphosphonic anhydride solution (50 wt. % in ethyl acetate, 176 μL, 188.49 mg, 592.39 μmol) was added to the reaction mixture at 0° C. The reaction mixture was stirred at ambient temperature for 2 h. The crude mixture was purified by reverse phase chromatography (C18 column (100 g); 0% to 50% in acetonitrile in water (0.1% ammonium acetate) over 30 minutes, then steep gradient to 100% acetonitrile). The pure fraction was frozen and lyophilized to afford Compound 1 (143.5 mg, 150.39 μmol, 38.08% yield) as an off white solid compound. LCMS (ESI+): 931.3 [M+H], ¹H-NMR (400 MHz, DMSO-d₆) δ=12.60-12.33 (s, 1H), 10.79 (br s, 1H), 7.77-7.59 (m, 5H), 7.49 (d, J=3.6 Hz, 1H), 7.26 (d, J=3.6 Hz, 1H), 6.85 (t, J=9.2 Hz, 1H), 6.59-6.47 (m, 3H), 6.42 (br d, J=8.8 Hz, 1H), 6.15 (s, 1H), 5.78 (br d, J=7.6 Hz, 1H), 4.84-4.74 (m, 2H), 4.39 (s, 2H), 4.29-4.17 (m, 2H), 4.12-3.93 (m, 8H), 2.93-2.66 (m, 6H), 2.62-2.54 (m, 2H), 2.47 (br d, J=5.6 Hz, 2H), 2.22 (s, 2H), 2.09 (td, J=4.4, 8.0 Hz, 1H), 1.90-1.71 (m, 3H), 1.68-1.57 (m, 2H).

### Example 3. 2-(6,7-dihydro-5H-pyrrolo[1,2-c]imidazol-1-yl)-2-[6-[4-[2-[2-[(4R)-4-[3-(2,4-dioxohexahydropyrimidin-1-yl)-1-methyl-indazol-6-yl]-3,3-difluoro-1-piperidyl]acetyl]-2,6-diazaspiro[3.3]heptan-6-yl]phenyl]-4-fluoro-1-oxo-isoindolin-2-yl]-N-thiazol-2-yl-acetamide (Compound 2)

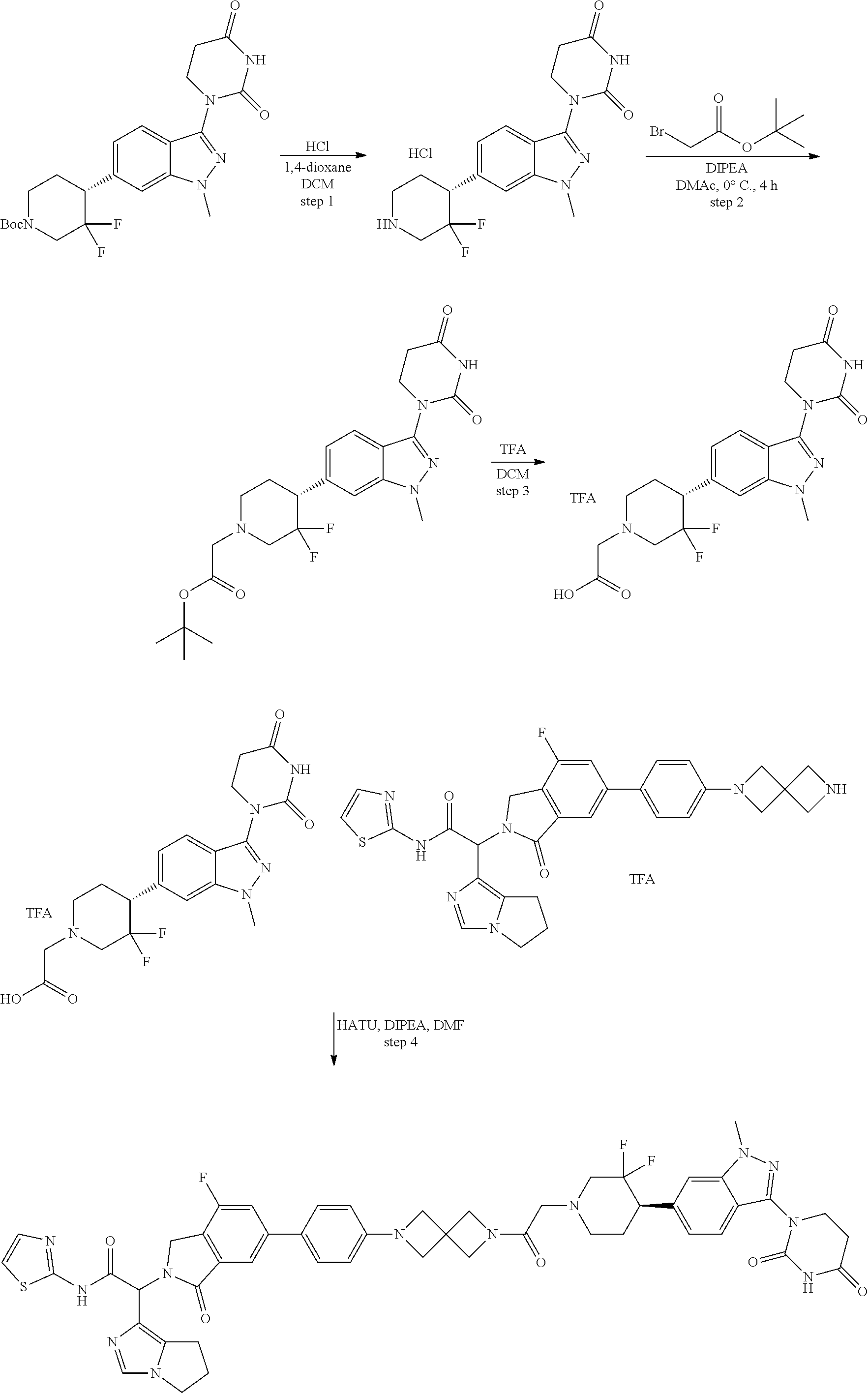

### Step 1: 1-[6-[(4R)-3,3-difluoro-4-piperidyl]-1-methyl-indazol-3-yl]hexahydropyrimidine-2,4-dione hydrochloride

tert-Butyl (4R)-4-[3-(2,4-dioxohexahydropyrimidin-1-yl)-1-methyl-indazol-6-yl]-3,3-difluoro-piperidine-1-carboxylate (Intermediate Z1, 325 mg, 701.22 μmol) was dissolved in a 1,4-dioxane:methanol mixture (1:1, 3 mL) and hydrogen chloride solution (4.0M in 1,4-dioxane, 3.51 mL, 14 mmol) was added. The reaction mixture was heated at 40° C. for 4 h. The volatiles were evaporated under reduce pressure. The solid residue was submitted to high vacuum to afford 1-[6-[(4R)-3,3-difluoro-4-piperidyl]-1-methyl-indazol-3-yl]hexahydropyrimidine-2,4-dione hydrochloride (280 mg, 665.30 μmol, 94.88% yield). LCMS (ESI+): 364.1 (M+H)

### Step 2: tert-Butyl 2-[(4R)-4-[3-(2,4-dioxohexahydropyrimidin-1-yl)-1-methyl-indazol-6-yl]-3,3-difluoro-1-piperidyl]acetate

1-[6-[(4R)-3,3-difluoro-4-piperidyl]-1-methyl-indazol-3-yl]hexahydropyrimidine-2,4-dione hydrochloride (285 mg, 784.34 μmol) and N,N-diisopropylethylamine (304.11 mg, 2.35 mmol, 409.85 μL) mixed in DMAc (0.5 mL). The reaction mixture was cooled to 0° C. tert-Butyl 2-bromoacetate (168.29 mg, 862.78 μmol, 126.53 μL) was added to the reaction mixture, and the mixture was warmed to 23° C. while stirring for 4 h. The reaction mixture was partitioned between ethyl acetate and sodium bicarbonate (aq., sat.). The organic layer was washed with brine, dried with sodium sulfate, filtered, and evaporated under reduced pressure. The crude residue was purified by silica gel chromatography (24 g column, 0% to 10% methanol in dichloromethane). Pure fractions were evaporated under reduced pressure to afford tert-butyl 2-[(4R)-4-[3-(2,4-dioxohexahydropyrimidin-1-yl)-1-methyl-indazol-6-yl]-3,3-difluoro-1-piperidyl]acetate (330 mg, 656.54 μmol, 83.71% yield). LCMS (ESI+): 478.2 (M+H)/422.2 (M-t-Bu+H)

### Step 3: 2-[(4R)-4-[3-(2,4-dioxohexahydropyrimidin-1-yl)-1-methyl-indazol-6-yl]-3,3-difluoro-1-piperidyl]acetic acid, trifluoroacetic acid salt

tert-Butyl 2-[(4R)-4-[3-(2,4-dioxohexahydropyrimidin-1-yl)-1-methyl-indazol-6-yl]-3,3-difluoro-1-piperidyl]acetate (330 mg, 691.09 μmol) was dissolved in a dichloromethane (2 mL) and Trifluoroacetic acid (1.42 g, 12.44 mmol, 958.39 μL) was added. The reaction mixture was heated at 40° C. for 4 h. The reaction mixture was cooled, added to methyl tert-butyl ether (20 mLs) under stirring at 0-5° C. The resulting suspension was stirred for 2 minutes. The suspension was transferred to a vial for centrifugation, and the suspension was centrifugated at 2400 rpm for 5 minutes. The supernatant solvent was decanted and discarded. methyl tert-butyl ether (20 mLs) was added the solid and the resulting suspension was stirred for 2 minutes. The suspension was transferred to a vial for centrifugation, and the suspension was centrifugated at 2400 rpm for 5 minutes. The supernatant solvent was decanted and discarded. The volatiles were evaporated in vacuo, and the solid was subjected to high vacuum for 1 h to afford 2-[(4R)-4-[3-(2,4-dioxohexahydropyrimidin-1-yl)-1-methyl-indazol-6-yl]-3,3-difluoro-1-piperidyl]acetic acid, trifluoroacetic acid salt (150 mg, 274.55 μmol, 39.73% yield). LCMS (ESI+): 422.2 (M+H)

### Step 4: 2-(6,7-dihydro-5H-pyrrolo[1,2-c]imidazol-1-yl)-2-[6-[4-[2-[2-[(4R)-4-[3-(2,4-dioxohexahydropyrimidin-1-yl)-1-methyl-indazol-6-yl]-3,3-difluoro-1-piperidyl]acetyl]-2,6-diazaspiro[3.3]heptan-6-yl]phenyl]-4-fluoro-1-oxo-isoindolin-2-yl]-N-thiazol-2-yl-acetamide

2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide, trifluoroacetic acid salt (240 mg, 351.04 μmol) and 2-[(4R)-4-[3-(2,4-dioxohexahydropyrimidin-1-yl)-1-methyl-indazol-6-yl]-3,3-difluoro-1-piperidyl]acetic acid, trifluoroacetic acid salt (147.93 mg, 351.04 μmol) were mixed in N,N-dimethylformamide, the reaction mixture was cooled to 0° C. N,N-diisopropylethylamine (272.22 mg, 2.11 mmol, 366.87 μL) was added to the reaction mixture, and 1-[bis(dimethylamino)methyl ene]-1H-1,2,3-triazolo[4,5-b]pyridinium 3-oxide hexafluorophosphate (133.48 mg, 351.04 μmol) was added, and the reaction mixture was cooled at 4° C. for 16 h. Water (300 μL) was added to the reaction mixture and stirred for 2 h. The mixture was injected on a 100 g C18 column, and purified using a 0% to 100% acetonitrile in water (+0.1% trifluoroacetic acid) elution gradient. The pure fractions were pooled and partitioned between 20:80 iPrOH:chloroform and sodium bicarbonate (aq., sat.). The organic layer was washed with brine, dried with sodium sulfate, filtered, and evaporated under reduced pressure. The residue was purified by silica gel chromatography (24 g column, 0% to 20% methanol in dichloromethane). Pure fractions were evaporated under reduced pressure. The solid was dissolved in 70:30 acetonitrile:water, sonicated to solubilize, frozen and lyophilized to afford Compound 2 (150 mg, 151.07 μmol, 43.04% yield). LCMS (ESI+): 973.2 (M+H), ¹H-NMR (400 MHz, DMSO-d₆) δ 12.45 (s, 1H), 10.50 (s, 1H), 7.67 (d, J=1.3 Hz, 1H), 7.64 (dd, J=10.7, 1.4 Hz, 1H), 7.58 (d, J=8.5 Hz, 2H), 7.55-7.45 (m, 3H), 7.40 (d, J=3.5 Hz, 1H), 7.16 (s, 1H), 7.03 (d, J=8.5 Hz, 1H), 6.48 (d, J=8.5 Hz, 2H), 6.06 (s, 1H), 4.75 (d, J=17.7 Hz, 1H), 4.37 (s, 2H), 4.14 (d, J=17.7 Hz, 1H), 4.07-3.94 (m, 7H), 3.92 (s, 4H), 3.85 (t, J=6.7 Hz, 2H), 3.16 (qd, J=14.9, 14.3, 7.1 Hz, 5H), 2.92 (d, J=10.8 Hz, 1H), 2.69 (t, J=6.8 Hz, 3H), 2.65-2.45 (m, 2H), 2.41-2.30 (m, 1H), 2.28-2.11 (m, 1H), 1.84-1.72 (m, 1H).

### Absolute Configuration of Compound 2

The absolute configuration of Compound 2 was established by determining the absolute configuration of intermediate tert-Butyl (4R)-4-[3-(2,4-dioxohexahydropyrimidin-1-yl)-1-methyl-indazol-6-yl]-3,3-difluoro-piperidine-1-carboxylate by X-ray diffraction of a co-crystal with a cereblon protein construct.

### Preparation tert-Butyl (4R)-4-[3-(2,4-dioxohexahydropyrimidin-1-yl)-1-methyl-indazol-6-yl]-3,3-difluoro-piperidine-1-carboxylate and tert-Butyl (4S)-4-[3-(2,4-dioxohexahydropyrimidin-1-yl)-1-methyl-indazol-6-yl]-3,3-difluoro-piperidine-1-carboxylate by Chiral SFC Separation

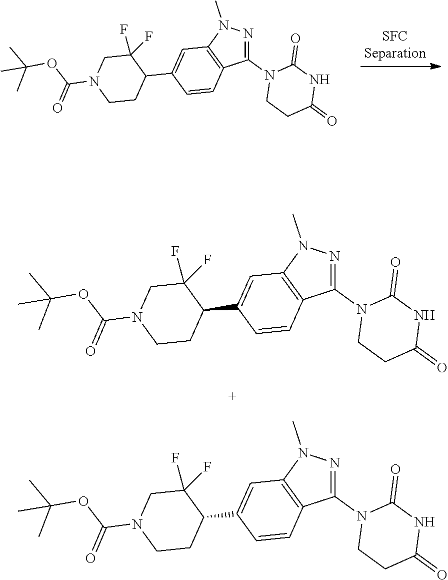 [SMILES: Cn1nc(N2CCC(=O)NC2=O)c2ccc(C3CCN(C(=O)OC(C)(C)C)CC3(F)F)cc21 ; Cn1nc(N2CCC(=O)NC2=O)c2ccc([C@H]3CCN(C(=O)OC(C)(C)C)CC3(F)F)cc21 ; Cn1nc(N2CCC(=O)NC2=O)c2ccc([C@@H]3CCN(C(=O)OC(C)(C)C)CC3(F)F)cc21]

Racemic tert-butyl 4-(3-(2,4-dioxotetrahydropyrimidin-1(2H)-yl)-1-methyl-1H-indazol-6-yl)-3,3-difluoropiperidine-1-carboxylate was resolved using SFC separation under the following conditions:
Sample Weight: 5.06 g
Column: ChiralCel OD-H 21×250 mm
Mobile Phase: 20% 2-Propanol in CO2
Flow Rate: 70 mL/min
Sample: Every 1 g sample was dissolved in 25 mL Ethanol and 25 mL Dichloromethane
Injection: 1 mL
Detection: 220 nm

The first eluting set of fractions was collected and evaporated to afford tert-Butyl (4R)-4-[3-(2,4-dioxohexahydropyrimidin-1-yl)-1-methyl-indazol-6-yl]-3,3-difluoro-piperidine-1-carboxylate (2.21 g, 43% yield, 100% ee) using the following analytical conditions. SFC retention time: 3.18 min (25% iso-propanol in supercritical CO₂, OD-H 4.6×100 mm, 40° C. 4 mL/min, 100 psi, 5 μL (Ethanol) injection), LCMS: 464 (M+H)

The absolute configuration of tert-Butyl (4R)-4-[3-(2,4-dioxohexahydropyrimidin-1-yl)-1-methyl-indazol-6-yl]-3,3-difluoro-piperidine-1-carboxylate was defined at ultra-high resolution via co-crystallisation with a Cereblon (CRBN) protein construct, followed by X-ray diffraction of both the protein and small molecule bound in situ. The structure resolution was 1 Å. The structure is shown with and without a density map overlay in FIG. 10 and FIG. 11.

The second eluting set of fractions was collected and evaporated to afford tert-Butyl (4S)-4-[3-(2,4-dioxohexahydropyrimidin-1-yl)-1-methyl-indazol-6-yl]-3,3-difluoro-piperidine-1-carboxylate (2.21 g, 43% yield, 100% ee) using the following analytical conditions. Retention time: 4.07 min (25% iso-propanol in supercritical CO2, OD-H 4.6×100 mm, 40° C. 4 mL/min, 100 psi, 5 μL (Ethanol) injection), LCMS: 464 (M+H).

### Example 4. Synthesis of 2-(6,7-dihydro-5H-pyrrolo[1,2-c]imidazol-1-yl)-2-[6-[4-[2-[2-[4-[3-(2,4-dioxohexahydropyrimidin-1-yl)-1-methyl-indazol-6-yl]-3,3-difluoro-1-piperidyl]acetyl]-2,6-diazaspiro[3.3]heptan-6-yl]phenyl]-4-fluoro-1-oxo-isoindolin-2-yl]-N-thiazol-2-yl-acetamide (Compound 3)

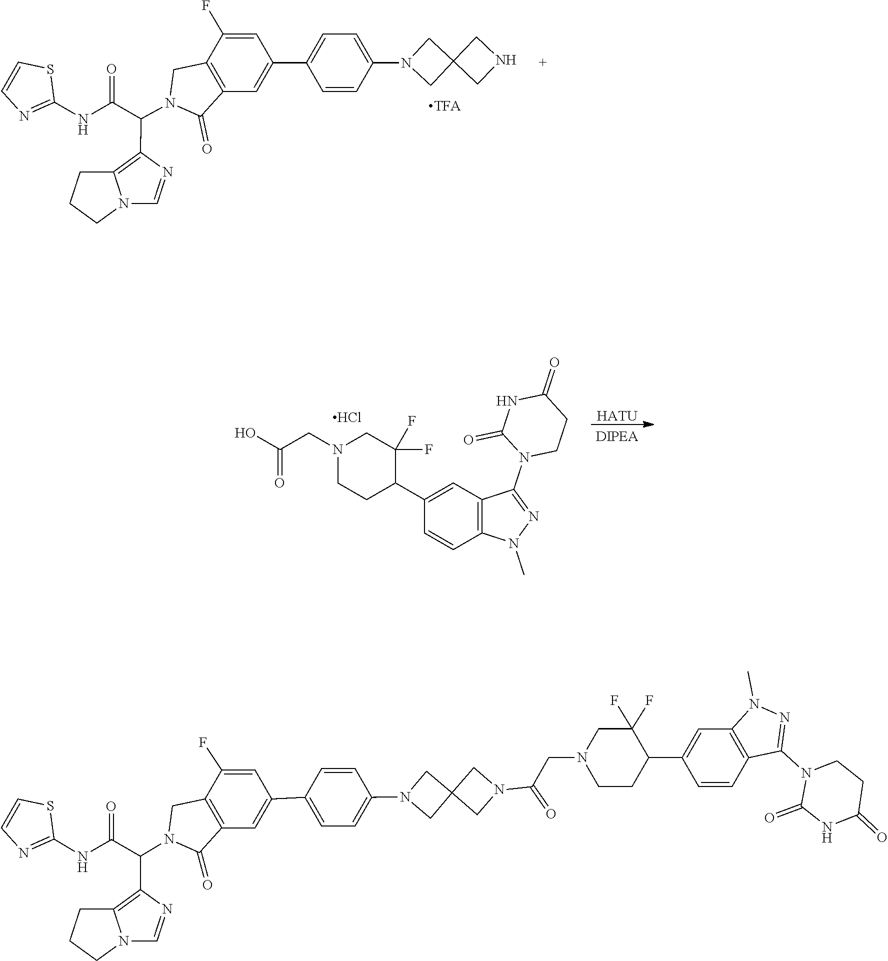

N,N-Diisopropylethylamine (47.26 mg, 365.67 μmol, 63.69 μL) was added to a solution of 2-[4-[3-(2,4-dioxohexahydropyrimidin-1-yl)-1-methyl-indazol-5-yl]-3,3-difluoro-1-piperidyl]acetic acid hydrochloride (43.53 mg, 95.07 μmol) in DMF (0.8 mL) at 0° C. HATU (30.59 mg, 80.45 μmol) was added at 0° C. and the mixture was stirred at ambient temperature for 10 min. Then, 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide trifluoroacetic acid salt (50 mg, 73.13 μmol) dissolved in DMF (0.4 mL) was added. The Reaction mixture was stirred for 30 minutes.

The mixture was injected on a 50 g C18 column, and purified using a 0% to 100% Acetonitrile in water+0.1% TFA water elution gradient. Desired fractions were pooled and partitioned between ethyl acetate and sodium bicarbonate (aqueous, aqueous). The organic layer was washed with brine, dried with sodium sulfate, filtered and evaporated under reduced pressure. The crude residue was purified by silica gel chromatography (0% to 20% methanol in dichloromethane). Pure fractions were evaporated to afford Compound 3 (30.3 mg, 29 μmol, 41% yield) as an off-white solid. LCMS (ESI+): 973.2 (M+H), 1H NMR (400 MHz, DMSO-d₆) δ 12.44 (s, 1H), 10.48 (s, 1H), 7.67 (d, J=1.4 Hz, 1H), 7.63 (dd, J=10.6, 1.4 Hz, 1H), 7.58 (d, J=8.6 Hz, 2H), 7.54-7.48 (m, 3H), 7.41 (d, J=3.5 Hz, 1H), 7.19 (d, J=3.6 Hz, 1H), 7.03 (d, J=8.5 Hz, 1H), 6.51-6.46 (m, 2H), 6.08 (s, 1H), 4.72 (d, J=17.7 Hz, 1H), 4.37 (s, 2H), 4.15 (d, J=17.7 Hz, 1H), 4.06-3.94 (m, 6H), 3.96-3.88 (m, 5H), 3.85 (t, J=6.7 Hz, 2H), 3.19-3.07 (m, 4H), 2.92 (d, J=10.8 Hz, 1H), 2.76-2.65 (m, 3H), 2.65-2.55 (m, 1H), 2.51-2.44 (m, 1H), 2.43-2.33 (m, 3H), 2.20 (dd, J=27.1, 14.4 Hz, 1H), 1.77 (m, 1H).

### Example 5. Synthesis of 2-(6,7-dihydro-5H-pyrrolo[1,2-c]imidazol-1-yl)-2-(6-(4-(6-(2-(4-(4-((2,6-dioxopiperidin-3-yl)amino)phenyl)-3,3-difluoropiperidin-1-yl)acetyl)-2,6-diazaspiro[3.3]heptan-2-yl)phenyl)-4-fluoro-1-oxoisoindolin-2-yl)-N-(thiazol-2-yl)acetamide, Isomer 1 (Compound 4)

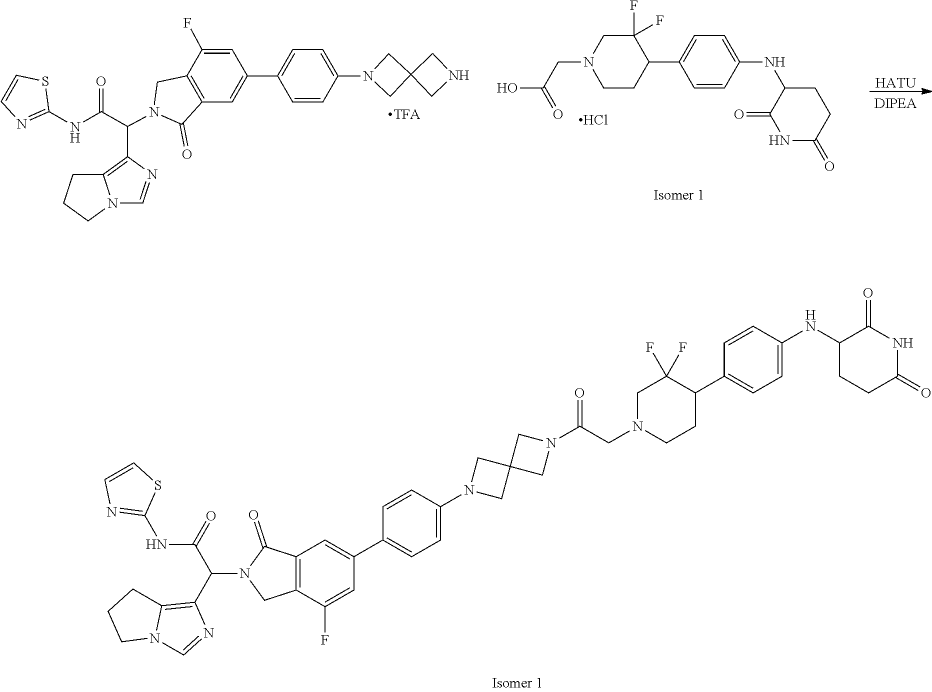

N,N-Diisopropylethylamine (56.71 mg, 438.81 μmol, 76.43 μL) was added to a solution of 2-[4-[4-[(2,6-dioxo-3-piperidyl)amino]phenyl]-3,3-difluoro-1-piperidyl]acetic acid hydrochloride isomer 1 (47.67 mg, 114.09 μmol) (47.67 mg, 114.09 μmol) in DMF (0.8 mL). HATU (36.71 mg, 96.54 μmol) was added at 0° C. and the mixture was stirred at ambient temperature for 10 min. 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide trifluoroacetic acid salt (60 mg, 87.76 μmol) dissolved in DMF (0.4 ml) was added. The reaction mixture was stirred for 2 hours. The mixture was injected on a 50 g C18 column, and purified using a 0% to 100% Acetonitrile in water+0.1% TFA water elution gradient. Desired fractions were pooled and partitioned between ethyl acetate and sodium bicarbonate (aqueous, aqueous). The organic layer was washed with brine, dried with sodium sulfate, filtered and evaporated under reduced pressure. The crude residue was purified by silica gel chromatography (24 g column, 0% to 20% methanol in ethyl acetate). Desired fractions were evaporated and the solid was dissolved in an acetonitrile:water mixture (1:1, 2 mL). The solution was frozen and lyophilized to afford Compound 4 (19.2 mg, 20.37 μmol, 23.2%). LCMS (ESI+): 933.3 (M+H), 1H NMR (400 MHz, DMSO-d₆) δ 12.52 (s, 1H), 10.78 (s, 1H), 7.75 (d, J=1.4 Hz, 1H), 7.71 (dd, J=10.7, 1.4 Hz, 1H), 7.68-7.62 (m, 2H), 7.61 (s, 1H), 7.49 (d, J=3.6 Hz, 1H), 7.26 (d, J=3.5 Hz, 1H), 7.02 (d, J=8.2 Hz, 2H), 6.64 (d, J=8.6 Hz, 2H), 6.55 (d, J=8.6 Hz, 2H), 6.15 (s, 1H), 5.81 (d, J=7.5 Hz, 1H), 4.80 (d, J=17.7 Hz, 1H), 4.42 (s, 2H), 4.30 (ddd, J=12.0, 7.5, 4.8 Hz, 1H), 4.22 (d, J=17.7 Hz, 1H), 4.10 (s, 2H), 4.07-3.93 (m, 6H), 3.21-3.08 (m, 3H), 2.97-2.87 (m, 2H), 2.88-2.67 (m, 3H), 2.67-2.44 (m, 4H), 2.42-2.30 (m, 1H), 2.16-1.97 (m, 2H), 1.89 (tt, J=12.1, 6.2 Hz, 1H), 1.71 (d, J=13.1 Hz, 1H).

### Example 6. Synthesis of 2-(6,7-dihydro-5H-pyrrolo[1,2-c]imidazol-1-yl)-2-[6-[4-[2-[2-[4-[3-(2,4-dioxohexahydropyrimidin-1-yl)-1-methyl-indazol-6-yl]-3,3-difluoro-1-piperidyl]acetyl]-2,6-diazaspiro[3.3]heptan-6-yl]phenyl]-4-fluoro-1-oxo-isoindolin-2-yl]-N-(2-pyridyl)acetamide (Compound 5)

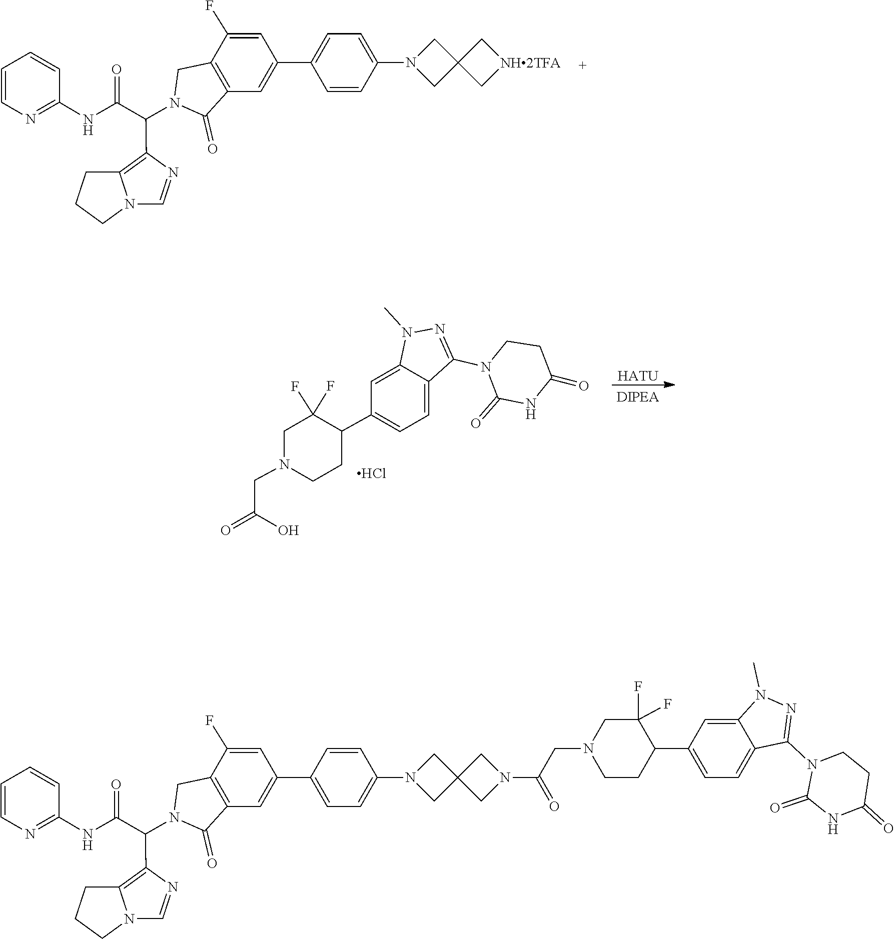

N,N-diisopropylethylamine (53.06 mg, 410.55 μmol, 71.51 μL) was added to 2-[4-[3-(2,4-Dioxohexahydropyrimidin-1-yl)-1-methyl-indazol-6-yl]-3,3-difluoro-1-piperidyl]acetic acid hydrochloride (45.11 mg, 98.53 μmol) (55.64 mg, 82.11 μmol) in DMF (0.5 mL). HATU (34.34 mg, 90.32 μmol) was added at 0° C. and stirred at ambient temperature for 10 min. 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-(2-pyridyl)acetamide; trifluoroacetic acid salt in DMF (0.4 ml) was added. The reaction mixture was stirred for 2 hours while warming to 20° C. The mixture was injected on a 50 g C18 column and purified using a 0% to 100% Acetonitrile in water+0.1% TFA water elution gradient. Desired fractions were neutralized with sodium bicarbonate (aqueous, aqueous), and the aqueous mixture was extracted twice with a 1:4 isopropanol:chloroform mixture. The organic layer was washed with brine, dried with sodium sulfate, filtered and evaporated under reduced pressure. The residue was purified by silica gel chromatography (0% to 20% methanol in ethyl acetate) to afford Compound 5 (47.3 mg, 48.42 μmol, 58.98% yield). LCMS (ESI+): 967.3 (M+H), 1H NMR (400 MHz, DMSO-d₆) δ 10.96 (s, 1H), 10.61 (s, 1H), 8.38 (dd, J=5.3, 1.9 Hz, 1H), 8.13 (d, J=8.4 Hz, 1H), 7.86 (ddd, J=8.8, 7.3, 2.0 Hz, 1H), 7.81 (d, J=1.3 Hz, 1H), 7.76 (dd, J=10.7, 1.4 Hz, 1H), 7.73-7.68 (m, 2H), 7.66 (d, J=8.5 Hz, 1H), 7.62 (s, 1H), 7.21-7.14 (m, 2H), 6.64-6.59 (m, 2H), 6.26 (s, 1H), 4.86 (d, J=17.7 Hz, 1H), 4.51 (s, 2H), 4.28 (d, J=17.7 Hz, 1H), 4.17 (s, 2H), 4.10 (d, J=5.9 Hz, 4H), 4.08-4.02 (m, 4H), 3.98 (t, J=6.7 Hz, 2H), 3.33-3.21 (m, 4H), 3.06 (d, J=10.9 Hz, 1H), 2.91-2.79 (m, 3H), 2.78-2.68 (m, 1H), 2.64-2.46 (m, 2H), 2.41-2.27 (m, 1H), 1.91 (d, J=12.5 Hz, 1H).

### Synthesis of 2-(6,7-dihydro-5H-pyrrolo[1,2-c]imidazol-1-yl)-2-[6-[4-[[4-[2-[4-[4-[(2,6-dioxo-3-piperidyl)amino]phenyl]-1-piperidyl]acetyl]piperazin-1-yl]methyl]phenyl]-4-fluoro-1-oxo-isoindolin-2-yl]-N-thiazol-2-yl-acetamide Compound 6

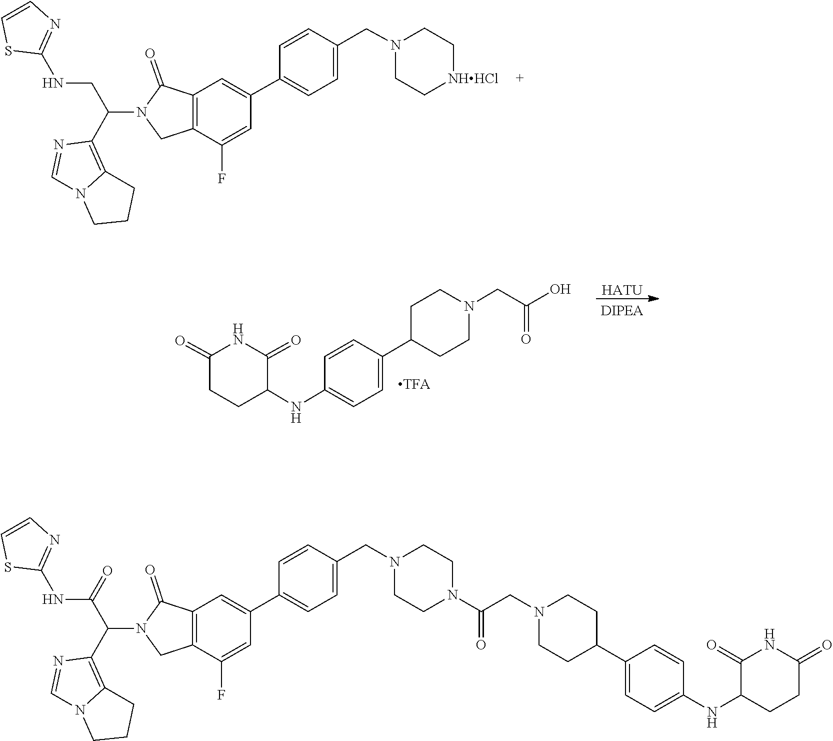

2-(6,7-Dihydro-5H-pyrrolo[1,2-c]imidazol-1-yl)-2-[4-fluoro-1-oxo-6-[4-(piperazin-1-ylmethyl)phenyl]isoindolin-2-yl]-N-thiazol-2-yl-acetamide dihydrochloride (60 mg, 93.08 μmol, 022) and 2-[4-[4-[(2,6-dioxo-3-piperidyl)amino]phenyl]-1-piperidyl]acetic acid, trifluoroacetic acid (42.76 mg, 93.08 μmol) were mixed in DMF, the reaction mixture was cooled to 0° C. N,N-Diisopropylethylamine (60.15 mg, 465.41 μmol, 81.06 μL) was added to the reaction mixture, and HATU (46.01 mg, 121.01 μmol) was added, and the reaction mixture was stirred for 4 hours while warming to room temperature. The reaction mixture was acidified with 4-5 drops of TFA, and injected directly on a RP C18 column (50 g C18) for purification using a 5% to 100% acetonitrile (+0.1% TFA) in water (+0.1% TFA) eluent gradient. The pure fractions were neutralized with aqueous NaHCO₃ (ca. 60 mL), extracted with 1:4 isopropanol:chloroform mixture. The organic layer was evaporated under reduced pressure to afford a solid. The solid was purified by silica gel chromatography (0% to 20% methanol in dichloromethane). The desired fractions were evaporated under reduced pressure. The residue was dissolved in dichloromethane, transferred to a 8 mL vial, and evaporated under reduced pressure. Water (1 mL) and acetonitrile (1 mL) were added, and the mixture was thoroughly sonicated, vortexed and sonicated again. The suspension was frozen and lyophilized to afford Compound 6 (10.5 mg, 11.21 μmol, 12.05% yield, 96% purity). LCMS (ESI+): 899.4 (M+H), ¹H NMR (400 MHz, DMSO-d₆) δ 12.53 (s, 1H), 10.76 (s, 1H), 7.98-7.70 (m, 4H), 7.61 (s, 1H), 7.49 (d, J=3.5 Hz, 1H), 7.45 (d, J=8.1 Hz, 2H), 7.37 (s, 2H), 7.26 (d, J=3.6 Hz, 1H), 6.95 (d, J=8.4 Hz, 2H), 6.61 (d, J=8.4 Hz, 2H), 6.16 (s, 1H), 5.64 (d, J=7.5 Hz, 1H), 4.84 (d, J=17.8 Hz, 1H), 4.41-4.16 (m, 2H), 4.15-3.87 (m, 3H), 3.74-3.52 (m, 4H), 3.47 (s, 2H), 3.14 (s, 2H), 2.88 (d, J=10.6 Hz, 2H), 2.84-2.64 (m, 2H), 2.63-2.53 (m, 1H), 2.48-2.19 (m, 4H), 2.22-1.94 (m, 3H), 1.95-1.77 (m, 1H), 1.77-1.63 (m, 2H), 1.63-1.42 (m, 1H).

### Example 7. Synthesis of 2-(6,7-dihydro-5H-pyrrolo[1,2-c]imidazol-1-yl)-2-[6-[4-[4-[2-[4-[5-[(2,6-dioxo-3-piperidyl)amino]-2-pyridyl]-1-piperidyl]acetyl]piperazin-1-yl]phenyl]-4-fluoro-1-oxo-isoindolin-2-yl]-N-thiazol-2-yl-acetamide (Compound 11)

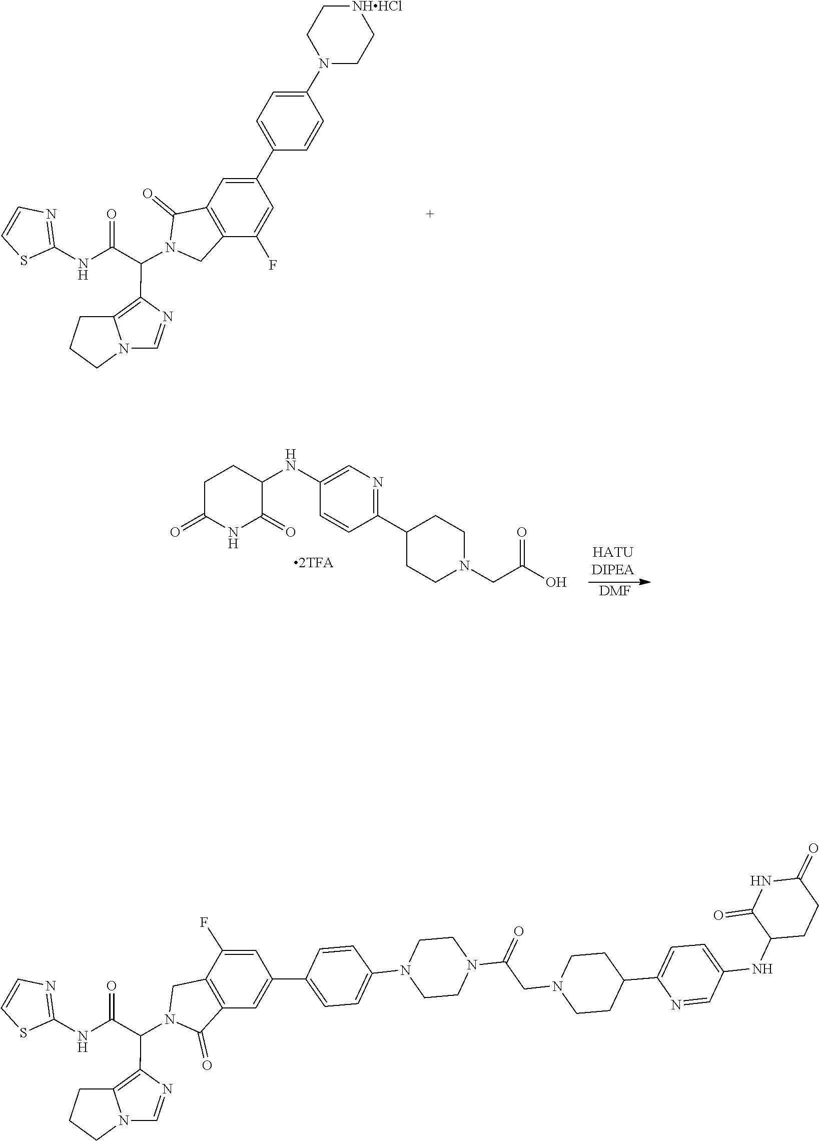

2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide; hydrochloride (75 mg, 126.24 μmol) and 2-[4-[5-[(2,6-dioxo-3-piperidyl)amino]-2-pyridyl]-1-piperidyl]acetic acid bis(trifluoroacetic acid) salt (79.77 mg, 138.87 μmol) were mixed in DMF, the reaction mixture was cooled to 0° C. N,N-Diisopropylethylamine (81.58 mg, 631.21 μmol, 109.94 μL) was added to the reaction mixture, and HATU (62.40 mg, 164.11 μmol) was added, and the reaction mixture was stirred for 4 hours while warming to room temperature. The reaction mixture was acidified with 4-5 drops of TFA and injected directly on a RP C18 column (50 g C18) for purification using a 5% to 100% acetonitrile (+0.1% TFA) in water (+0.1% TFA) eluent gradient. The pure fractions were neutralized with aqueous NaHCO₃(60 mL), extracted with a isopropanol:chloroform mixture (1:4). The organic layer was evaporated under reduced pressure to afford a solid. The solid was dissolved in dichloromethane, an injected on a 24 g silica gel column flushed with 100% dichloromethane and purified using a 0% to 20% methanol in dichloromethane gradient over 20 minutes. The pure fractions were evaporated under reduced pressure. The crude residue in dichloromethane was transferred to an 8 mL vial, and evaporated under reduced pressure. The compound was suspended in an acetonitrile:water mixture, and the mixture was thoroughly sonicated, vortexed and sonicated again. The suspension was frozen and lyophilized to afford Compound 11 (35 mg, 39.11 μmol, 30.98% yield). LCMS (ESI+): 886.6 (M+H). 1H NMR (400 MHz, DMSO-d₆) δ 12.52 (s, 1H), 10.78 (s, 1H), 7.97 (t, J=1.8 Hz, 1H), 7.78 (d, J=1.3 Hz, 1H), 7.76 (s, 1H), 7.72-7.65 (m, 2H), 7.61 (s, 1H), 7.49 (d, J=3.6 Hz, 1H), 7.26 (d, J=3.5 Hz, 1H), 7.08 (d, J=8.8 Hz, 2H), 6.97 (d, J=1.8 Hz, 2H), 6.16 (s, 1H), 5.92 (d, J=7.8 Hz, 1H), 4.81 (d, J=17.7 Hz, 1H), 4.33 (ddd, J=12.2, 7.8, 4.9 Hz, 1H), 4.23 (d, J=17.7 Hz, 1H), 4.11-3.85 (m, 2H), 3.70 (d, J=58.2 Hz, 4H), 3.21 (s, 4H), 2.93 (d, J=10.7 Hz, 2H), 2.85-2.65 (m, 2H), 2.67-2.52 (m, 1H), 2.49 (m, 2H), 2.12 (m, 2H), 1.90 (qd, J=12.3, 4.7 Hz, 1H), 1.84-1.48 (m, 4H).

### Example 8. Synthesis of 2-(6,7-dihydro-5H-pyrrolo[1,2-c]imidazol-1-yl)-2-[6-[4-[4-[2-[4-[4-[(2,6-dioxo-3-piperidyl)amino]-2-fluoro-phenyl]-1-piperidyl]acetyl]piperazin-1-yl]phenyl]-4-fluoro-1-oxo-isoindolin-2-yl]-N-thiazol-2-yl-acetamide (Compound 12)

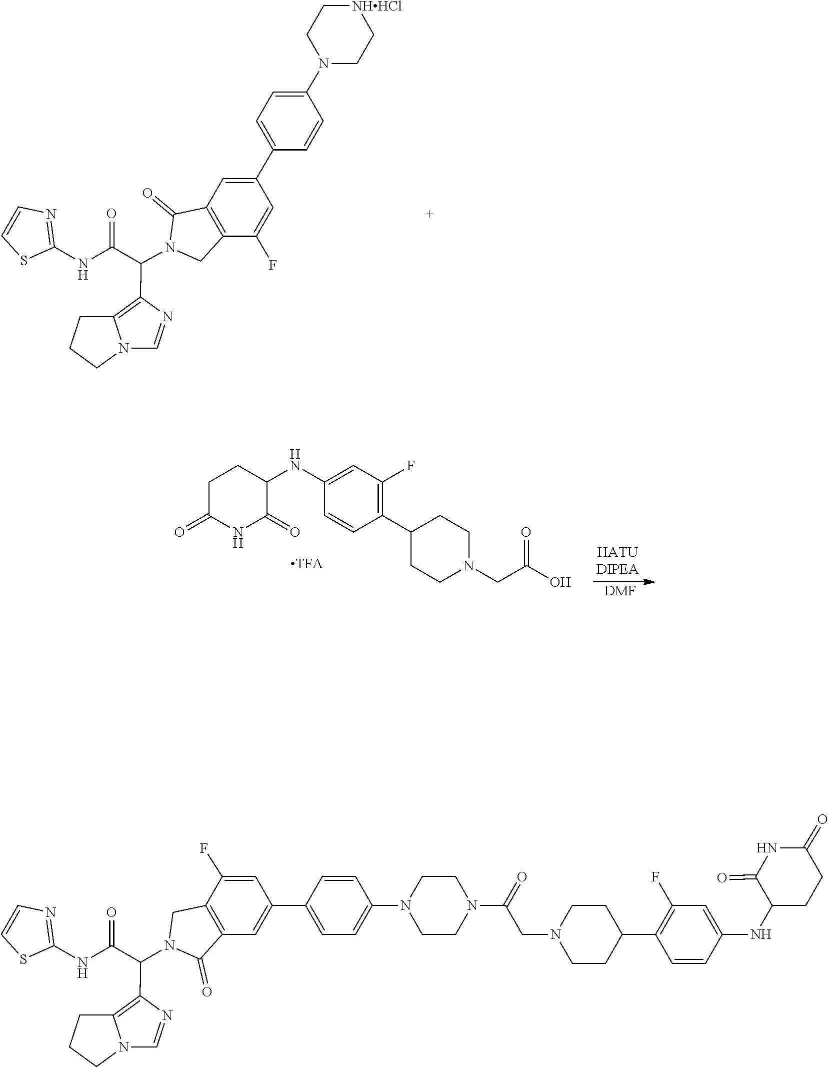

2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide; hydrochloride (51.85 mg, 87.28 μmol) and 2-[4-[4-[(2,6-dioxo-3-piperidyl)amino]-2-fluoro-phenyl]-1-piperidyl]acetic acid, trifluoroacetic acid salt (50 mg, 104.73 μmol) were mixed in DMF, the reaction mixture was cooled to 0° C. N,N-Diisopropylethylamine (56.40 mg, 436.39 μmol, 76.01 μL) was added to the reaction mixture, and HATU (43.14 mg, 113.46 μmol) was added, and the reaction mixture was stirred for 4 hours while warming to room temperature. The reaction mixture was acidified with 4-5 drops of TFA, and injected directly on a RP C18 column (50 g C18) for purification using a 5% to 100% acetonitrile (+0.1% TFA) in water (+0.1% TFA) eluent gradient. The pure fractions were neutralized with saturated aqueous sodium bicarbonate (60 mL), extracted with 1:4 isopropanol:chloroform mixture. The organic layer was evaporated under reduced pressure to afford a solid. The solid was dissolved in dichloromethane, an injected on a 24 g silica gel column flushed with 100% dichloromethane, and purified using a 0% to 20% methanol in dichloromethane gradient over 20 minutes. The pure fractions were evaporated under reduced pressure. The crude residue was dissolved in dichloromethane, transferred to a 8 mL vial, and evaporated under reduced pressure. 1 mL water+1 mL acetonitrile were added, and the mixture was thoroughly sonicated, vortexed and sonicated again. The suspension was frozen and lyophilized to afford Compound 12 (20 mg, 21.48 μmol, 24.62% yield). LCMS (ESI+): 903.7 (M+H). ¹H NMR (400 MHz, DMSO-d₆) δ 12.51 (s, 1H), 10.77 (s, 1H), 7.93-7.63 (m, 4H), 7.60 (s, 1H), 7.48 (d, J=3.6 Hz, 1H), 7.36 (s, 1H), 7.25 (d, J=3.6 Hz, 1H), 7.14-7.02 (m, 2H), 6.98 (t, J=8.8 Hz, 1H), 6.58-6.32 (m, 2H), 6.15 (s, 1H), 5.99 (d, J=7.7 Hz, 1H), 4.80 (d, J=17.7 Hz, 1H), 4.30 (td, J=7.5, 3.9 Hz, 1H), 4.22 (d, J=17.7 Hz, 1H), 3.98 (m, 2H), 3.85-3.45 (m, 4H), 3.25 (d, J=34.8 Hz, 6H), 2.93 (d, J=10.7 Hz, 2H), 2.83-2.65 (m, 2H), 2.64-2.51 (m, 2H), 2.47 (m, 1H), 2.18-1.97 (m, 3H), 1.95-1.76 (m, 1H), 1.66 (m, 4H).

### Example 9. Synthesis of 2-(6,7-dihydro-5H-pyrrolo[1,2-c]imidazol-1-yl)-2-[6-[4-[4-[2-[4-[4-[(2,6-dioxo-3-piperidyl)amino]-3-fluoro-phenyl]-1-piperidyl]acetyl]piperazin-1-yl]phenyl]-4-fluoro-1-oxo-isoindolin-2-yl]-N-thiazol-2-yl-acetamide (Compound 13)

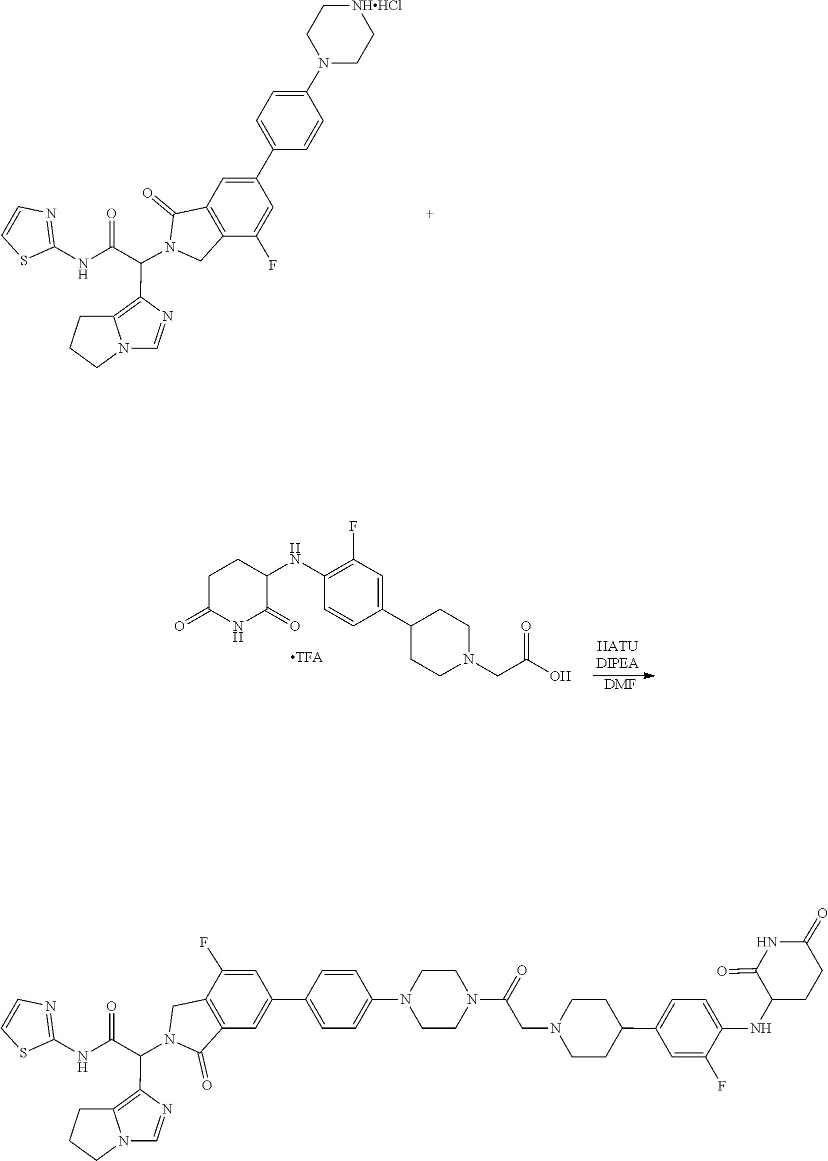

2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide; hydrochloride (48.5 mg, 81.64 μmol) and 2-[4-[4-[(2,6-dioxo-3-piperidyl)amino]-3-fluoro-phenyl]-1-piperidyl]acetic acid, trifluoroacetic acid salt (46.77 mg, 97.96 μmol) were mixed in DMF, the reaction mixture was cooled to 0° C. N,N-Diisopropylethylamine (52.75 mg, 408.18 μmol, 71.10 μL) was added to the reaction mixture, and HATU (40.35 mg, 106.13 μmol) was added, and the reaction mixture was stirred for 4 hours while warming to room temperature. The reaction mixture was acidified with 4-5 drops of TFA, and injected directly on a RP C18 column (50 g C18) for purification (5% to 100% acetonitrile+0.1% TFA in water+0.1% TFA over 12 minutes). The pure fractions were neutralized with aqueous NaHCO₃(60 mL), extracted with a isopropanol:chloroform mixture (1:4). The organic layer was evaporated under reduced pressure to afford a solid. The solid was dissolved in dichloromethane, an injected on a 24 g silica gel column flushed with 100% dichloromethane, and purified using a 0% to 20% methanol in dichloromethane gradient over 20 minutes. The pure fractions were evaporated under reduced pressure. The crude residue was dissolved in dichloromethane, transferred to a 8 mL vial, and evaporated under reduced pressure. The compound was dissolved in a water:acetonitrile mixture (1 mL:1 mL) were added, and the mixture was thoroughly sonicated, vortexed and sonicated again. The suspension was frozen and lyophilized to afford Compound 13 (15.5 mg, 16.65 μmol, 20.40% yield). LCMS (ESI+): 903.6 (M+H), ¹H NMR (400 MHz, DMSO-d₆) δ 12.52 (s, 1H), 10.80 (s, 1H), 7.78 (d, J=1.3 Hz, 1H), 7.74 (dd, J=10.6, 1.4 Hz, 1H), 7.69 (d, J=8.8 Hz, 2H), 7.61 (s, 1H), 7.49 (d, J=3.6 Hz, 1H), 7.37 (s, 2H), 7.26 (d, J=3.6 Hz, 1H), 7.08 (d, J=8.9 Hz, 2H), 6.94 (dd, J=13.3, 1.9 Hz, 1H), 6.84 (dd, J=8.4, 1.9 Hz, 1H), 6.75 (t, J=8.9 Hz, 1H), 6.15 (s, 1H), 5.38 (dd, J=7.9, 2.4 Hz, 1H), 4.80 (d, J=17.7 Hz, 1H), 4.35 (ddd, J=12.6, 7.9, 5.2 Hz, 1H), 4.23 (d, J=17.7 Hz, 1H), 4.10-3.88 (m, 2H), 3.69 (d, J=54.2 Hz, 4H), 3.26 (d, J=31.5 Hz, 6H), 2.93 (d, J=10.7 Hz, 2H), 2.83-2.69 (m, 1H), 2.63-2.52 (m, 1H), 2.50-2.25 (m, 2H), 2.25-1.88 (m, 4H), 1.73 (d, J=12.1 Hz, 2H), 1.58 (q, J=12.1 Hz, 2H).

### General Procedure A1 for the Coupling of 2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide hydrochloride with acids

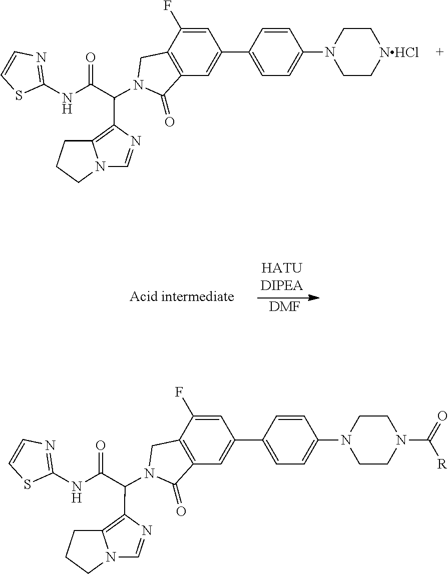

2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide hydrochloride (1 equiv.) and Appropriate Acid intermediate (1.2 equiv.) were mixed in DMF (0.2 M), the reaction mixture was cooled to 0° C. N,N-Diisopropylethylamine (5 equiv.) was added to the reaction mixture, and HATU (1.3 equiv.) was added, and the reaction mixture was stirred for 4 hours while warming to room temperature. The reaction mixture was acidified with 4-5 drops of TFA, and injected directly on a RP C18 column (50 g C18) for purification using a 5% to 100% acetonitrile (+0.1% TFA) in water (+0.1% TFA) eluent gradient. The pure fractions were neutralized with aqueous NaHCO₃, extracted with an isopropanol:chloroform (1:4) mixture. The organic layer was evaporated under reduced pressure to afford a solid. The solid was dissolved in dichloromethane, and injected on a 24 g silica gel column flushed with 100% dichloromethane and purified using a 0% to 20% methanol in dichloromethane gradient over 20 minutes. The pure fractions were evaporated under reduced pressure. The crude residue was dissolved in dichloromethane, transferred to a 8 mL vial, and evaporated under reduced pressure. The compound was suspended in an acetonitrile:water mixture and the mixture was thoroughly sonicated and vortexed. The suspension was frozen and lyophilized to afford the title compound.

### General Procedure B1 for the Coupling of 2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide hydrochloride with acids

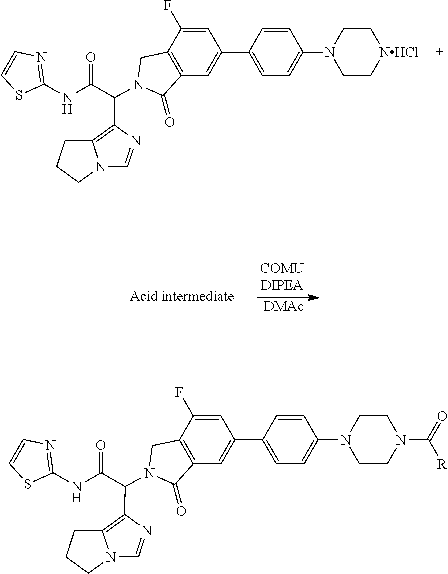

To a solution of 2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide (1 equiv.) and Appropriate Acid intermediate (1.2 equiv.) in DMAc (0.2 M) was added N,N-Diisopropylethylamine (34.77 mg, 268.99 μmol, 46.85 μL) and (1-Cyano-2-ethoxy-2-oxoethylidenaminooxy)dimethylamino-morpholino-carbenium hexafluorophosphate (57.60 mg, 134.50 μmol) at 0° C. the reaction mixture was stirred for 3 hours. The volatiles were evaporated under reduced pressure and the crude was purified by prep HPLC under the following conditions: Column: Agilent preparative C18 (50*21.2 mm, 5 μm). Eluent mixture: 10 mM Ammonium acetate in water:Acetonitrile. Pure fractions were lyophilized to afford the title compound.

### General Procedure for Coupling of acids to 2-(6,7-dihydro-5H-pyrrolo[1,2-c]imidazol-1-yl)-2-(4-fluoro-1-oxo-6-(4-(piperidin-4-yl)phenyl)isoindolin-2-yl)-N-(thiazol-2-yl)acetamide

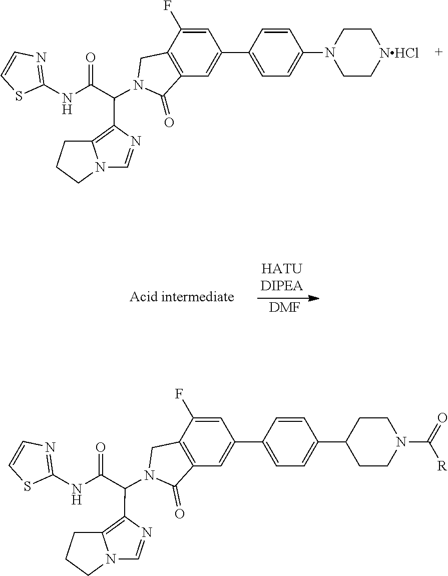

2-(6,7-dihydro-5H-pyrrolo[1,2-c]imidazol-1-yl)-2-(4-fluoro-1-oxo-6-(4-(piperidin-4-yl)phenyl)isoindolin-2-yl)-N-(thiazol-2-yl)acetamide (1 equiv.) and Appropriate Acid intermediate (1.2 equiv.) were mixed in DMF and the reaction mixture was cooled to 0° C. N,N-Diisopropylethylamine (5 equiv.) was added to the reaction mixture, and HATU (1.3 equiv.) was added, and the reaction mixture was stirred for 4 hours while warming to room temperature. The reaction mixture was acidified with 4-5 drops of TFA, and injected directly on a C18 column (50 g C18) for purification (5% to 100% acetonitrile (+0.1% TFA) in water (+0.1% TFA) over 12 minutes). The pure fractions were neutralized with aqueous NaHCO₃ (ca. 60 mL), extracted with 1:4 isopropanol:chloroform (1:1) mixture. The organic layer was evaporated under reduced pressure to afford a solid. The solid was dissolved in dichloromethane, and injected on a 24 g silica gel column flushed with 100% dichloromethane, and purified using a 0% to 20% methanol in dichloromethane gradient over 20 minutes. The pure fractions were evaporated under reduced pressure. The crude residue was dissolved in dichloromethane, transferred to a 8 mL vial, and evaporated under reduced pressure. Water (1 mL) and acetonitrile (1 mL) were added, and the mixture was thoroughly sonicated, vortexed and sonicated again. The suspension was frozen and lyophilized to afford the title compound.

### Example 10. 2-(6,7-dihydro-5H-pyrrolo[1,2-c]imidazol-1-yl)-2-[6-[4-[[1-[2-[4-[4-[(2,6-dioxo-3-piperidyl)amino]phenyl]-1-piperidyl]acetyl]-4-piperidyl]oxy]phenyl]-4-fluoro-1-oxo-isoindolin-2-yl]-N-thiazol-2-yl-acetamide (Compound 31)

### Step 1: tert-butyl 4-[4-[2-[1-(6,7-dihydro-5H-pyrrolo[1,2-c]imidazol-1-yl)-2-ethoxy-2-oxo-ethyl]-7-fluoro-3-oxo-isoindolin-5-yl]phenoxy]piperidine-1-carboxylate

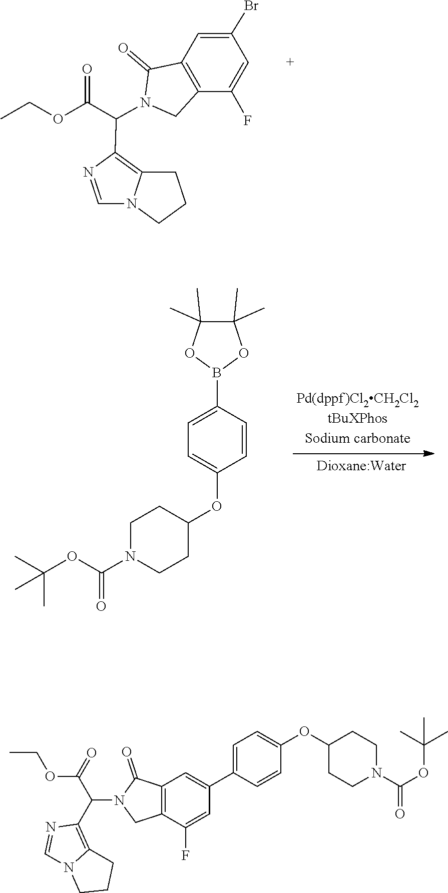 [SMILES: CCOC(=O)C(c1ncn2c1CCC2)N1Cc2c(F)cc(Br)cc2C1=O ; CC(C)(C)OC(=O)N1CCC(Oc2ccc(B3OC(C)(C)C(C)(C)O3)cc2)CC1 ; CCOC(=O)C(c1ncn2c1CCC2)N1Cc2c(F)cc(-c3ccc(OC4CCN(C(=O)OC(C)(C)C)CC4)cc3)cc2C1=O]

In a 100-mL round bottom flask, ethyl 2-(6-bromo-4-fluoro-1-oxo-isoindolin-2-yl)-2-(6,7-dihydro-5H-pyrrolo[1,2-c]imidazol-1-yl)acetate (3.1 g, 7.34 mmol) and tert-butyl 4-[4-(4,4,5,5-tetramethyl-1,3,2-dioxaborolan-2-yl)phenoxy]piperidine-1-carboxylate (4.00 g, 9.91 mmol) were dissolved in dioxane (44 mL) and Pd(dppf)Cl₂·CH₂Cl₂ (299.77 mg, 367.08 μmol) and tBuXPhos (463.44 mg, 734.17 μmol) were added, followed by Sodium carbonate (1.71 g, 16.15 mmol) dissolved in Water (11 mL). The mixture was degassed with nitrogen. The reaction was capped with a septum, fitted with a nitrogen inlet, and heated at 80° C. on a heating block for 5 h. The mixture was diluted with ethyl acetate, and the organic layer was separated from the aqueous layer as well as the solid precipitate. The crude residue purified by flash column chromatography on silica gel (0-10% Methanol in ethyl acetate) to give tert-butyl 4-[4-[2-[1-(6,7-dihydro-5H-pyrrolo[1,2-c]imidazol-1-yl)-2-ethoxy-2-oxo-ethyl]-7-fluoro-3-oxo-isoindolin-5-yl]phenoxy]piperidine-1-carboxylate (3.6 g, 5.82 mmol, 79.26% yield) as a pale orange foam. LCMS: 619.4 (M+H)

### Step 2: [2-[6-[4-[(1-tert-butoxycarbonyl-4-piperidyl)oxy]phenyl]-4-fluoro-1-oxo-isoindolin-2-yl]-2-(6,7-dihydro-5H-pyrrolo[1,2-c]imidazol-1-yl)acetyl]

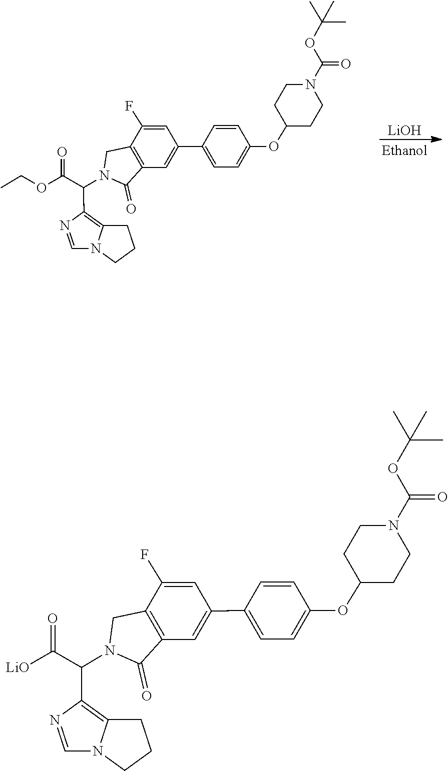

Lithium hydroxide (1 M, 5.82 mL) was added to a solution of tert-butyl 4-[4-[2-[1-(6,7-dihydro-5H-pyrrolo[1,2-c]imidazol-1-yl)-2-ethoxy-2-oxo-ethyl]-7-fluoro-3-oxo-isoindolin-5-yl]phenoxy]piperidine-1-carboxylate (3.6 g, 5.82 mmol) in Ethanol (25 ml). The reaction mixture was heated to 40° C. for 16 h. The reaction mixture was concentrated in vacuo, suspended in benzene and evaporated The residue was submitted to high vacuum to afford [2-[6-[4-[(1-tert-butoxycarbonyl-4-piperidyl)oxy]phenyl]-4-fluoro-1-oxo-isoindolin-2-yl]-2-(6,7-dihydro-5H-pyrrolo[1,2-c]imidazol-1-yl)acetyl]oxylithium (3.47 g, quantitative yield). LCMS (ESI+): 590.9 (M+H)

### Step 3: tert-butyl 4-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]piperidine-1-carboxylate

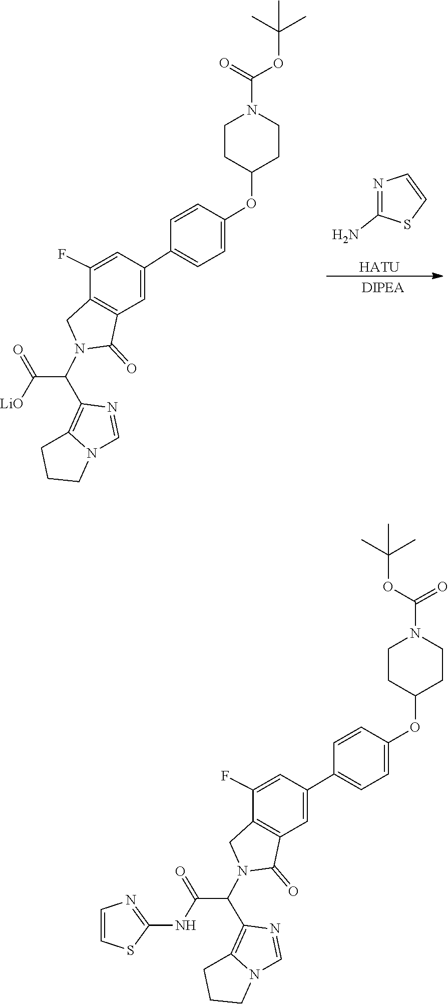

[2-[6-[4-[(1-tert-butoxycarbonyl-4-piperidyl)oxy]phenyl]-4-fluoro-1-oxo-isoindolin-2-yl]-2-(6,7-dihydro-5H-pyrrolo[1,2-c]imidazol-1-yl)acetyl]oxylithium (3.47 g, 5.82 mmol) and thiazol-2-amine (640.73 mg, 6.40 mmol) were mixed in DMF and the reaction mixture was cooled to 0° C. N,N-Diisopropylethylamine (3.01 g, 23.27 mmol, 4.05 mL) was added to the reaction mixture, and HATU (2.88 g, 7.56 mmol) was added, and the reaction mixture was stirred for 30 min at 0° C. Saturated aqueous sodium bicarbonate was added to the reaction mixture. The reaction mixture was extracted with ethyl acetate (2×). The organic layers were washed with water, brine, dried over Na₂SO₄, filtered and concentrated under reduced pressure. The crude material was purified by flash chromatography on silica gel (24 g, 0-10% methanol in dichloromethane) to afford tert-butyl 4-[4-[2-[1-(6,7-dihydro-5H-pyrrolo[1,2-c]imidazol-1-yl)-2-oxo-2-(thiazol-2-yl amino)ethyl]-7-fluoro-3-oxo-isoindolin-5-yl]phenoxy]piperidine-1-carboxylate (3 g, 4.46 mmol, 77% yield). LCMS (ESI+): 673.2 (M+H)

### Step 4: 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride

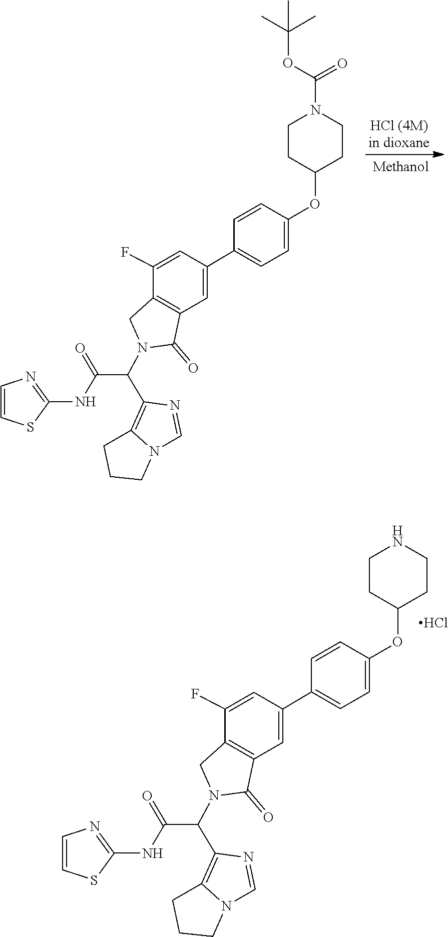 [SMILES: CC(C)(C)OC(=O)N1CCC(Oc2ccc(-c3cc(F)c4c(c3)C(=O)N(C(C(=O)Nc3nccs3)c3ncn5c3CCC5)C4)cc2)CC1 ; O=C(Nc1nccs1)C(c1ncn2c1CCC2)N1Cc2c(F)cc(-c3ccc(OC4CCNCC4)cc3)cc2C1=O ; Cl]

tert-Butyl 4-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]piperidine-1-carboxylate (3 g, 4.46 mmol) was dissolved in 1,4-dioxane (10 mL) and methanol (10 mL). A hydrogen chloride solution (4.0M in 1,4-dioxane, 8 mL, 32 mmol) was added. The reaction mixture was heated at 40° C. for 4 hours. The volatiles were evaporated under reduced pressure. The material was submitted to high vacuum, frozen to −78° C. and thawed to afford 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride (3.2 g, 5.25 mmol, quantitative yield) as a dense solid.

### Step 5: 2-(6,7-dihydro-5H-pyrrolo[1,2-c]imidazol-1-yl)-2-[6-[4-[[1-[2-[4-[4-[(2,6-dioxo-3-piperidyl)amino]phenyl]-1-piperidyl]acetyl]-4-piperidyl]oxy]phenyl]-4-fluoro-1-oxo-isoindolin-2-yl]-N-thiazol-2-yl-acetamide

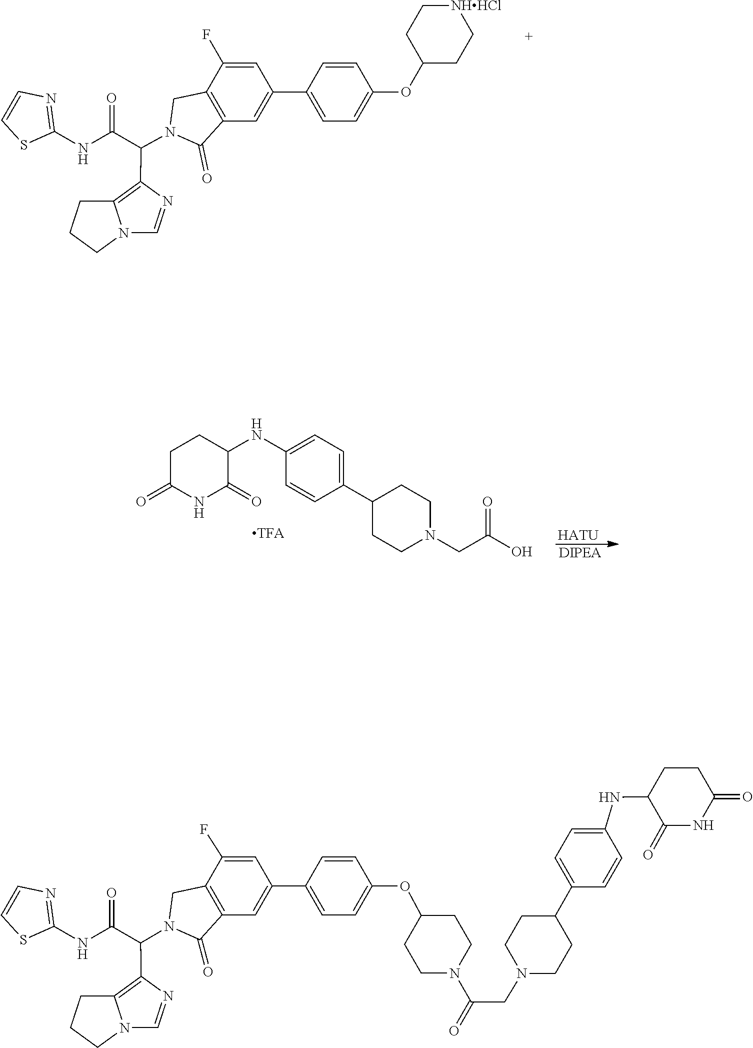

2-(6,7-Dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride (100 mg, 164.17 μmol) and 2-[4-[4-[(2,6-dioxo-3-piperidyl)amino]phenyl]-1-piperidyl]acetic acid trifluoroacetic acid salt (98.05 mg, 213.43 μmol) were mixed in DMF, the reaction mixture was cooled to 0° C. N,N-Diisopropylethylamine (106.09 mg, 820.87 μmol, 142.98 μL) was added to the reaction mixture, and HATU (81.15 mg, 213.43 μmol) was added, and the reaction mixture was stirred for 1 h in an ice bath. The reaction mixture was acidified with 4-5 drops of TFA, and injected directly on a RP C18 column (50 g C18) for purification using a 5% to 100% acetonitrile (+0.1% TFA) in water (+0.1% TFA) eluent gradient. The pure fractions were neutralized with aqueous NaHCO₃ (ca. 60 mL), extracted twice with a isopropanol:chloroform mixture (1;4). The organic layer was dried over sodium sulfate, filtered, and evaporated under reduced pressure to afford a solid. The solid was dissolved in dichloromethane, injected on a 24 g silica gel column flushed with 100% dichloromethane, and purified using a 0% to 20% methanol in dichloromethane gradient over 20 minutes. The pure fractions were evaporated under reduced pressure. The crude residue was dissolved in dichloromethane, transferred to an 8 mL vial, and evaporated under reduced pressure. Water (1 mL) and acetonitrile (1 mL) were added, and the mixture was thoroughly sonicated, vortexed and sonicated again. The suspension was frozen and lyophilized to afford Compound 31 (62 mg, 68.20 μmol, 41% yield) as an off-white solid. LCMS (ESI+): 900.7 (M+H), 1H NMR (400 MHz, DMSO-d₆) δ 12.53 (s, 1H), 10.76 (s, 1H), 7.82-7.74 (m, 3H), 7.76-7.71 (m, 1H), 7.61 (s, 1H), 7.49 (d, J=3.6 Hz, 1H), 7.26 (d, J=3.6 Hz, 1H), 7.15-7.08 (m, 2H), 7.00-6.93 (m, 2H), 6.66-6.59 (m, 2H), 6.16 (s, 1H), 5.65 (d, J=7.5 Hz, 1H), 4.82 (d, J=17.7 Hz, 1H), 4.74 (dq, J=8.1, 3.9 Hz, 1H), 4.32-4.20 (m, 2H), 3.99 (dddd, J=11.2, 8.2, 6.3, 3.1 Hz, 2H), 3.89 (d, J=12.0 Hz, 2H), 3.45 (t, J=10.2 Hz, 1H), 3.31-3.10 (m, 3H), 2.91 (d, J=10.7 Hz, 2H), 2.75 (ddt, J=23.3, 11.9, 5.7 Hz, 2H), 2.63-2.44 (m, 4H), 2.40-2.28 (m, 1H), 2.16-2.00 (m, 4H), 2.00-1.89 (m, 1H), 1.86 (qd, J=12.2, 4.8 Hz, 1H), 1.77-1.64 (m, 3H), 1.57 (q, J=12.2 Hz, 3H).

### Example 11. Synthesis of 2-(6,7-dihydro-5H-pyrrolo[1,2-c]imidazol-1-yl)-2-[6-[4-[[1-[2-[4-[4-[[(3S)-2,6-dioxo-3-piperidyl]amino]phenyl]-1-piperidyl]acetyl]-4-piperidyl]oxy]phenyl]-4-fluoro-1-oxo-isoindolin-2-yl]-N-thiazol-2-yl-acetamide (Compound 32)

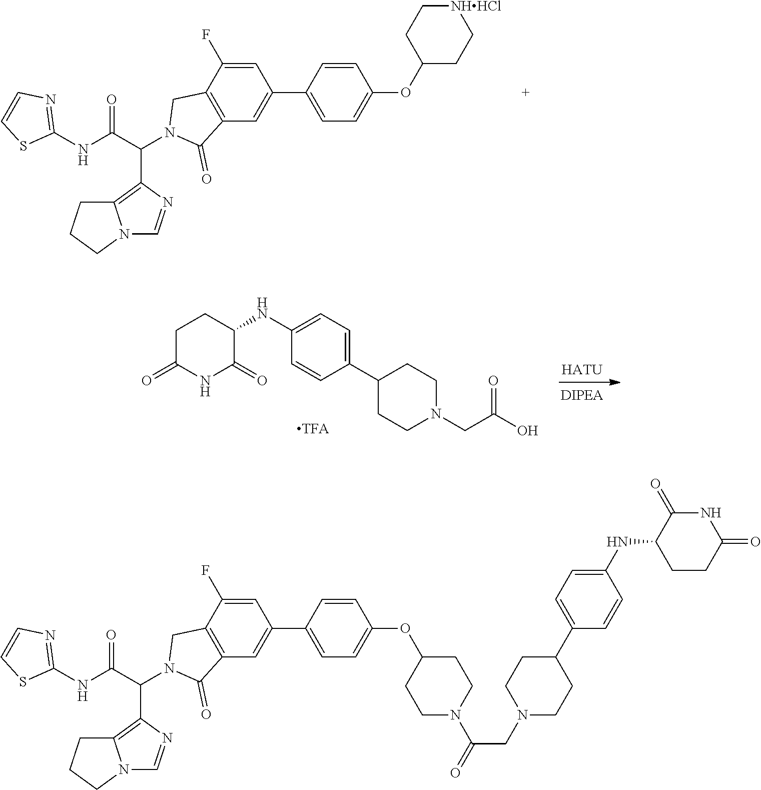

Compound 32 was prepared in 47% yield using the same procedure as 2-(6,7-dihydro-5H-pyrrolo[1,2-c]imidazol-1-yl)-2-[6-[4-[[1-[2-[4-[4-[[2,6-dioxo-3-piperidyl]amino]phenyl]-1-piperidyl]acetyl]-4-piperidyl]oxy]phenyl]-4-fluoro-1-oxo-isoindolin-2-yl]-N-thiazol-2-yl-acetamide, using 2-[4-[4-[[(3S)-2,6-dioxo-3-piperidyl]amino]phenyl]-1-piperidyl]acetic acid trifluoroacetic acid salt instead of 2-[4-[4-[[2,6-dioxo-3-piperidyl]amino]phenyl]-1-piperidyl]acetic acid trifluoroacetic acid salt. LCMS (ESI+): 900.3 (M+H), 1H NMR (400 MHz, DMSO-d₆) δ 12.52 (s, 1H), 10.76 (s, 1H), 7.82-7.72 (m, 4H), 7.61 (s, 1H), 7.49 (d, J=3.6 Hz, 1H), 7.26 (d, J=3.6 Hz, 1H), 7.12 (d, J=8.5 Hz, 2H), 6.96 (d, J=8.2 Hz, 2H), 6.62 (d, J=8.2 Hz, 2H), 6.16 (s, 1H), 5.65 (d, J=7.5 Hz, 1H), 4.82 (d, J=17.7 Hz, 1H), 4.77-4.68 (m, 1H), 4.32-4.19 (m, 2H), 4.08-3.95 (s, 2H), 3.95-3.83 (m, 2H), 3.52-3.08 (m, 4H), 2.92 (m, 2H), 2.83-2.67 (m, 2H), 2.64-2.42 (m, 4H), 2.40-2.28 (m, 1H), 2.18-2.02 (m, 4H), 1.99-1.79 (m, 2H), 1.79-1.65 (m, 3H), 1.63-1.50 (m, 3H).

### Example 12. Synthesis of 2-(6,7-dihydro-5H-pyrrolo[1,2-c]imidazol-1-yl)-2-[6-[4-[[1-[2-[4-[4-[[(3R)-2,6-dioxo-3-piperidyl]amino]phenyl]-1-piperidyl]acetyl]-4-piperidyl]oxy]phenyl]-4-fluoro-1-oxo-isoindolin-2-yl]-N-thiazol-2-yl-acetamide (Compound 33)

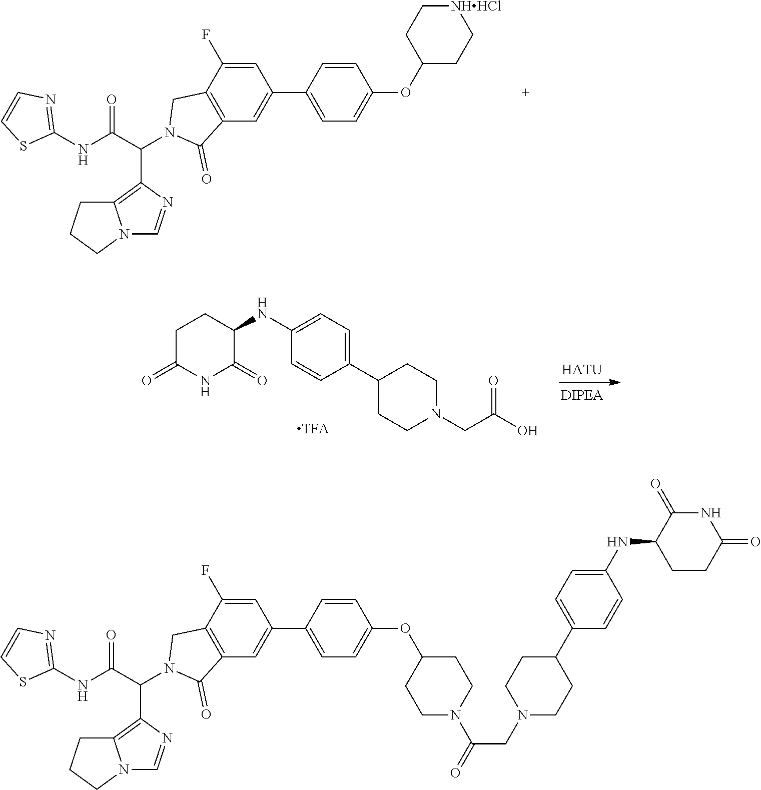

Compound 33 was prepared in 36% yield using the same procedure as 2-(6,7-dihydro-5H-pyrrolo[1,2-c]imidazol-1-yl)-2-[6-[4-[[1-[2-[4-[4-[[2,6-dioxo-3-piperidyl]amino]phenyl]-1-piperidyl]acetyl]-4-piperidyl]oxy]phenyl]-4-fluoro-1-oxo-isoindolin-2-yl]-N-thiazol-2-yl-acetamide, using 2-[4-[4-[[(3R)-2,6-dioxo-3-piperidyl]amino]phenyl]-1-piperidyl]acetic acid trifluoroacetic acid salt instead of 2-[4-[4-[[2,6-dioxo-3-piperidyl]amino]phenyl]-1-piperidyl]acetic acid trifluoroacetic acid salt. (54 mg, 59.40 μmol, 36.18% yield). LCMS (ESI+): 900.6 (M+H), ¹H NMR (400 MHz, DMSO-d₆) δ 12.52 (s, 1H), 10.76 (s, 1H), 7.82-7.73 (m, 4H), 7.61 (s, 1H), 7.49 (d, J=3.5 Hz, 1H), 7.26 (d, J=3.6 Hz, 1H), 7.15-7.09 (m, 2H), 6.96 (d, J=8.2 Hz, 2H), 6.62 (d, J=8.2 Hz, 2H), 6.16 (s, 1H), 5.65 (d, J=7.5 Hz, 1H), 4.82 (d, J=17.8 Hz, 1H), 4.74 (m, 1H), 4.32-4.21 (m, 2H), 4.06-3.95 (m, 2H), 3.89 (m, 2H), 3.46 (m, 1H), 3.33-3.10 (m, 3H), 2.91 (d, J=10.5 Hz, 2H), 2.82-2.66 (m, 3H), 2.62-2.42 (m, 3H), 2.33 (dd, J=3.8, 1.9 Hz, 1H), 2.17-2.01 (m, 4H), 1.88 (ddd, J=20.1, 15.0, 9.8 Hz, 2H), 1.77-1.64 (m, 3H), 1.64-1.49 (m, 3H).

### General Method A for Amide Coupling of 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride to Acid Intermediates

The compounds were synthesized from 2-(6,7-dihydro-5H-pyrrolo[1,2-c]imidazol-1-yl)-2[4-fluoro-1-oxo-6-[4-(4-piperidyloxy)phenyl]isoindolin-2-yl]-N-thiazol-2-yl-acetamide hydrochloride using the method used in for the synthesis Example 10, Step 5.

### General Method B for Amide Coupling of 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride to Acid Intermediates

To a solution of 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride (1 equiv.) and acid intermediate (1.1 equiv.) in DMF (0.1 M) was added N,N-diisopropylethylamine (5 equiv.) followed by HATU (1.3 equiv.) at ambient temperature. The reaction was further stirred at ambient temperature for 16 h. The reaction mixture was quenched with ice cold water and extracted with 10% methanol in dichloromethane (50 ml). Concentrated organic layer under reduced pressure afforded crude. Purified crude on Reverse phase column eluting compound in 40-50% acetonitrile in Water (with 0.1% TFA phase modifier). Pure fractions were lyophilized to afford a solid, which was further purified by Prep HPLC. Purification method: Column: Zorbax Extend C18 (50×4.6 mm) 5 μm, Mobile Phase A:10 mM Ammonium acetate in water, Mobile Phase B: Acetonitrile. The pure fractions were frozen and lyophilized to afford the title compound.

### Example 13. Synthesis of 2-(6,7-dihydro-5H-pyrrolo[1,2-c]imidazol-1-yl)-2-(6-(4-((1-(2-(4-(4-((2,6-dioxopiperidin-3-yl)amino)-3-fluorophenyl)piperidin-1-yl)acetyl)piperidin-4-yl)oxy)phenyl)-4-fluoro-1-oxoisoindolin-2-yl)-N-(thiazol-2-yl)acetamide (Compound 34)

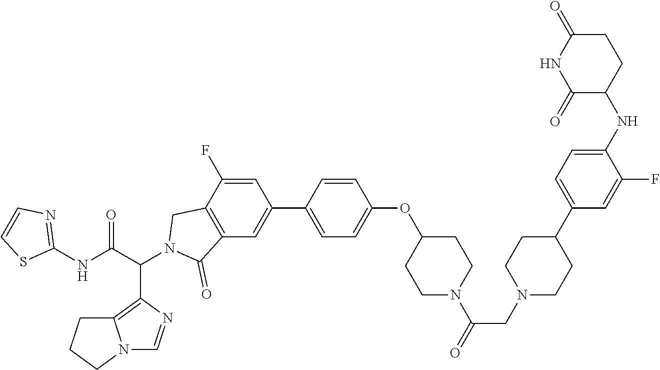 [SMILES: O=C1CCC(Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)cc2F)C(=O)N1]

Synthesized according to General Method A for Amide Coupling of 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride to Acid Intermediates in 52% Yield.

LCMS (ESI+) 918.7 (M+H)

¹H NMR (400 MHz, DMSO-d₆) δ 12.52 (s, 1H), 10.78 (s, 1H), 7.82-7.70 (m, 4H), 7.61 (s, 1H), 7.49 (d, J=3.6 Hz, 1H), 7.26 (d, J=3.6 Hz, 1H), 7.15-7.09 (m, 2H), 6.99 (t, J=8.8 Hz, 1H), 6.49-6.40 (m, 2H), 6.16 (s, 1H), 6.00 (d, J=7.8 Hz, 1H), 4.82 (d, J=17.7 Hz, 1H), 4.73 (t, J=3.7 Hz, 1H), 4.31 (td, J=7.4, 3.8 Hz, 1H), 4.24 (d, J=17.8 Hz, 1H), 4.06-3.95 (m, 2H), 3.89 (m, 2H), 3.51-3.40 (m, 1H), 3.30-3.08 (m, 3H), 2.92 (d, J=10.7 Hz, 2H), 2.83-2.66 (m, 2H), 2.62-2.41 (m, 3H), 2.15-2.02 (m, 5H), 1.88-1.79 (m, 1H), 1.87 (qd, J=12.3, 4.6 Hz, 3H), 1.74-1.59 (m, 6H), 1.59-1.47 (m, 1H).

### Example 14. Synthesis of 2-(6,7-dihydro-5H-pyrrolo[1,2-c]imidazol-1-yl)-2-(6-(4-((1-(2-(4-(4-((2,6-dioxopiperidin-3-yl)amino)-2-fluorophenyl)piperidin-1-yl)acetyl)piperidin-4-yl)oxy)phenyl)-4-fluoro-1-oxoisoindolin-2-yl)-N-(thiazol-2-yl)acetamide (Compound 35)

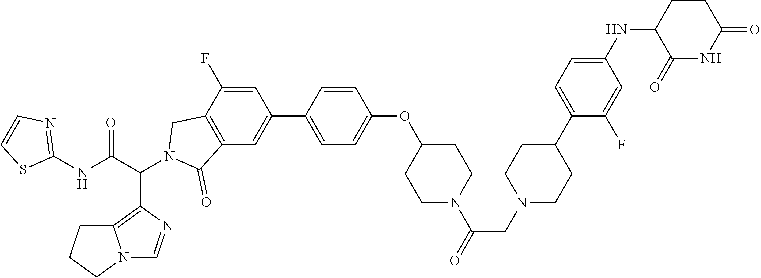 [SMILES: O=C1CCC(Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)c(F)c2)C(=O)N1]

Synthesized according to General Method A for Amide Coupling of 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride to Acid Intermediates in 52% Yield.

LCMS (ESI+) 918.7 (M+H)

¹H NMR (400 MHz, DMSO-d₆) δ 12.52 (s, 1H), 10.78 (s, 1H), 7.82-7.70 (m, 4H), 7.61 (s, 1H), 7.49 (d, J=3.6 Hz, 1H), 7.26 (d, J=3.6 Hz, 1H), 7.15-7.09 (m, 2H), 6.99 (t, J=8.8 Hz, 1H), 6.49-6.40 (m, 2H), 6.16 (s, 1H), 6.00 (d, J=7.8 Hz, 1H), 4.82 (d, J=17.7 Hz, 1H), 4.73 (t, J=3.7 Hz, 1H), 4.31 (td, J=7.4, 3.8 Hz, 1H), 4.24 (d, J=17.8 Hz, 1H), 4.06-3.95 (m, 2H), 3.89 (m, 2H), 3.51-3.40 (m, 1H), 3.30-3.08 (m, 3H), 2.92 (d, J=10.7 Hz, 2H), 2.83-2.66 (m, 2H), 2.62-2.41 (m, 3H), 2.15-2.02 (m, 5H), 1.88-1.79 (m, 1H), 1.87 (qd, J=12.3, 4.6 Hz, 3H), 1.74-1.59 (m, 6H), 1.59-1.47 (m, 1H).

### Example 15. Synthesis of 2-(6,7-dihydro-5H-pyrrolo[1,2-c]imidazol-1-yl)-2-(6-(4-((1-(2-(4-(5-((2,6-dioxopiperidin-3-yl)amino)pyridin-2-yl)piperidin-1-yl)acetyl)piperidin-4-yl)oxy)phenyl)-4-fluoro-1-oxoisoindolin-2-yl)-N-(thiazol-2-yl)acetamide (Compound 36)

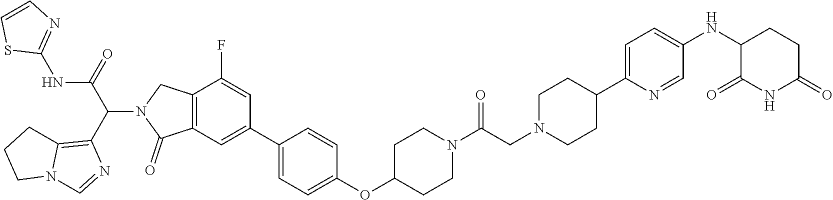 [SMILES: O=C1CCC(Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)nc2)C(=O)N1]

Synthesized according to General Method A for Amide Coupling of 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride to Acid Intermediates in 30% Yield.

LCMS (ESI+) 901.5 (M+H)

¹H NMR (400 MHz, DMSO-d₆) δ 12.52 (s, 1H), 10.79 (s, 1H), 7.98 (t, J=1.8 Hz, 1H), 7.82-7.72 (m, 4H), 7.61 (s, 1H), 7.49 (d, J=3.6 Hz, 1H), 7.26 (d, J=3.6 Hz, 1H), 7.15-7.09 (m, 2H), 6.98 (d, J=1.8 Hz, 2H), 6.16 (s, 1H), 5.93 (d, J=7.8 Hz, 1H), 4.82 (d, J=17.7 Hz, 1H), 4.78-4.69 (m, 1H), 4.34 (ddd, J=12.2, 7.8, 4.9 Hz, 1H), 4.24 (d, J=17.7 Hz, 1H), 4.06 −3.84 (m, 4H), 3.45 (t, J=10.0 Hz, 1H), 3.31-3.07 (m, 2H), 2.91 (d, J=10.7 Hz, 2H), 2.83-2.67 (m, 2H), 2.64-2.42 (m, 6H), 2.17-2.01 (m, 4H), 2.00-1.83 (m, 2H), 1.82-1.60 (m, 5H), 1.60-1.47 (m, 1H).

### Example 16. Synthesis of 2-(6-(4-((1-(2-(4-(2-cyano-44(2,6-dioxopiperidin-3-yl)amino)phenyl)piperidin-1-yl)acetyl)piperidin-4-yl)oxy)phenyl)-4-fluoro-1-oxoisoindolin-2-yl)-2-(6,7-dihydro-5H-pyrrolo[1,2-c]imidazol-1-yl)-N-(thiazol-2-yl)acetamide (Compound 37)

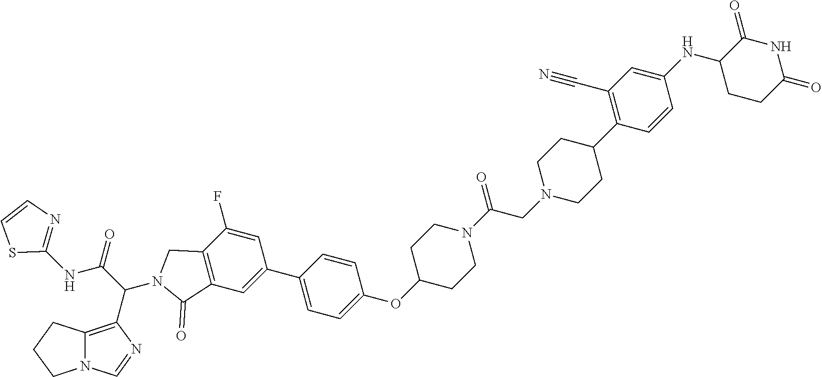 [SMILES: N#Cc1cc(NC2CCC(=O)NC2=O)ccc1C1CCN(CC(=O)N2CCC(Oc3ccc(-c4cc(F)c5c(c4)C(=O)N(C(C(=O)Nc4nccs4)c4ncn6c4CCC6)C5)cc3)CC2)CC1]

Synthesized according to General Method A for Amide Coupling of 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride to Acid Intermediates in 46% Yield.

LCMS (ESI+) 925.5 (M+H)

¹H NMR (400 MHz, DMSO-d₆) δ 12.52 (s, 1H), 10.80 (s, 1H), 7.82-7.71 (m, 4H), 7.62 (s, 1H), 7.49 (d, J=3.6 Hz, 1H), 7.26 (d, J=3.6 Hz, 1H), 7.21 (d, J=8.7 Hz, 1H), 7.15-7.09 (m, 2H), 6.97 (d, J=7.6 Hz, 2H), 6.25 (d, J=7.9 Hz, 1H), 6.16 (s, 1H), 4.82 (d, J=17.7 Hz, 1H), 4.78-4.70 (m, 1H), 4.40 (ddd, J=12.3, 7.9, 4.9 Hz, 1H), 4.24 (d, J=17.7 Hz, 1H), 4.00 (ddt, J=10.5, 7.5, 4.6 Hz, 2H), 3.93-3.83 (m, 2H), 3.46 (t, J=10.4 Hz, 1H), 3.30-3.13 (m, 3H), 2.96 (d, J=10.2 Hz, 2H), 2.83-2.64 (m, 3H), 2.64-2.45 (m, 3H), 2.20-2.02 (m, 4H), 2.02-1.83 (m, 2H), 1.70 (s, 5H), 1.55 (d, J=8.8 Hz, 1H).

### Example 17. Synthesis of 2-(6,7-dihydro-5H-pyrrolo[1,2-c]imidazol-1-yl)-2-(6-(4-((1-(2-(4-(4-((2,4-dioxo-3-azabicyclo[3.1.1]heptan-1-yl)amino)phenyl)piperidin-1-yl)acetyl)piperidin-4-yl)oxy)phenyl)-4-fluoro-1-oxoisoindolin-2-yl)-N-(thiazol-2-yl)acetamide (Compound 38)

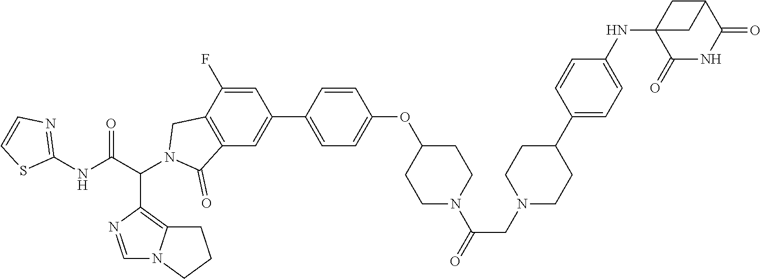 [SMILES: O=C1NC(=O)C2(Nc3ccc(C4CCN(CC(=O)N5CCC(Oc6ccc(-c7cc(F)c8c(c7)C(=O)N(C(C(=O)Nc7nccs7)c7ncn9c7CCC9)C8)cc6)CC5)CC4)cc3)CC1C2]

Synthesized according to General Method B for Amide Coupling of 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride to Acid Intermediates in 4.47% Yield.

LCMS (ESI+) 912.2 (M+H)

¹H-NMR (400 MHz, DMSO-d₆): δ 12.53 (s, 1H), 10.71 (s, 1H), 7.78 (dd, J=1.2, 12.4 Hz, 4H), 7.62 (s, 1H), 7.49 (d, J=3.6 Hz, 1H), 7.27 (d, J=3.6 Hz, 1H), 7.12 (d, J=8.8 Hz, 2H), 6.92 (d, J=8.4 Hz, 2H), 6.40 (d, J=8.4 Hz, 2H), 6.15 (s, 1H), 6.08 (s, 1H), 4.82 (d, J=17.6 Hz, 1H), 4.77-4.68 (m, 1H), 4.24 (d, J=17.6 Hz, 1H), 4.05-3.98 (m, 2H), 3.97-3.88 (m, 2H), 3.28-3.27 (m, 3H), 2.97-2.81 (m, 4H), 2.80-2.71 (m, 3H), 2.15-2.01 (m, 4H), 1.95-1.88 (m, 2H), 1.75-1.61 (m, 4H), 1.61-1.49 (m, 4H).

### Example 18. Synthesis of 2-(6,7-dihydro-5H-pyrrolo[1,2-c]imidazol-1-yl)-2-(6-(4-((1-(2-(4-(4-(((S)-2,6-dioxopiperidin-3-yl)amino)-2-fluorophenyl)piperidin-1-yl)acetyl)piperidin-4-yl)oxy)phenyl)-4-fluoro-1-oxoisoindolin-2-yl)-N-(thiazol-2-yl)acetamide (Compound 39)

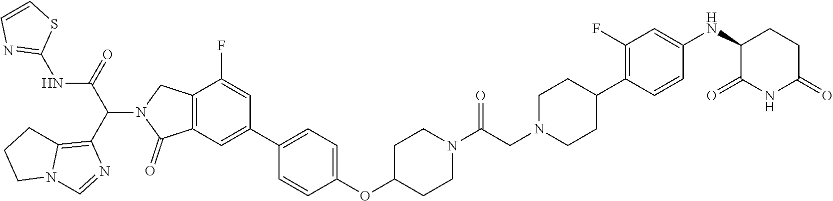 [SMILES: O=C1CC[C@H](Nc2ccc(C3CCN(CC(=O)N4CCC(Oc5ccc(-c6cc(F)c7c(c6)C(=O)N(C(C(=O)Nc6nccs6)c6ncn8c6CCC8)C7)cc5)CC4)CC3)c(F)c2)C(=O)N1]

Synthesized according to General Method B for Amide Coupling of 2-(6,7-dihydro-5H-pyrrolo[1,2-c]imidazol-1-yl)-2-[4-fluoro-1-oxo-6-[4-(4-piperidyloxy)phenyl]isoindolin-2-yl]-N-thiazol-2-yl-acetamide hydrochloride to Acid Intermediates in 39% Yield.

LCMS (ESI+) 919.0 (M+H)

¹H-NMR (400 MHz, DMSO-d₆): 12.56 (s, 1H), 10.79 (s, 1H), 7.80-7.73 (m, 4H), 7.62 (s, 1H), 7.49 (d, J=3.6 Hz, 1H), 7.27 (d, J=3.6 Hz, 1H), 7.12 (d, J=8.8 Hz, 2H), 6.99 (d, J=9.2 Hz, 1H), 6.47-6.43 (m, 2H), 6.16 (s, 1H), 6.02 (d, J=7.6 Hz, 1H), 4.84-4.74 (m, 2H), 4.31-4.22 (m, 2H), 4.03-3.97 (m, 4H), 3.48-3.41 (m, 1H), 3.34-3.12 (m, 3H), 2.92-2.88 (m, 2H), 2.78-2.67 (m, 2H), 2.61-2.54 (m, 3H), 2.11-2.06 (m, 4H), 1.92-1.86 (m, 3H), 1.76-1.51 (m, 7H).

### Example 19. Synthesis of 2-(6,7-dihydro-5H-pyrrolo[1,2-c]imidazol-1-yl)-2-[6-[4-[[1-[2-[4-[4-[(2,6-dioxo-3-piperidyl)amino]phenyl]-1-piperidyl]-2-oxo-acetyl]-4-piperidyl]oxy]phenyl]-4-fluoro-1-oxo-isoindolin-2-yl]-N-thiazol-2-yl-acetamide (Compound 40)

### Step 1: Ethyl 2-[4-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]-1-piperidyl]-2-oxo-acetate

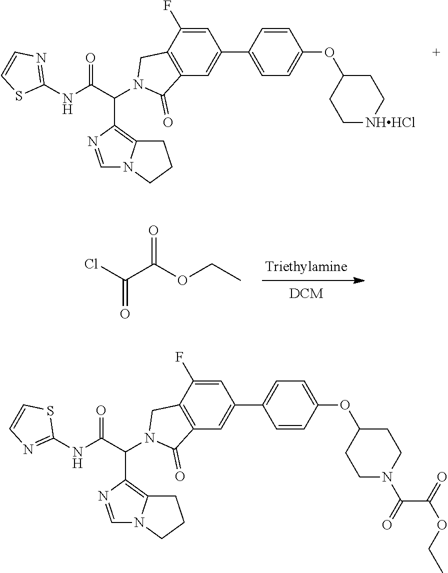

To a stirred solution of 2-(6,7-dihydro-5H-pyrrolo[1,2-c]imidazole-1-yl)-2-[4-fluoro-1-oxo-6-[4-(4-piperidyloxy)phenyl]isoindolin-2-yl]-N-thiazol-2-yl-acetamide; hydrochloride (58 mg, 95.22 μmol) in dichloromethane (1 mL) was added Triethylamine (24.09 mg, 238.05 μmol, 33.18 μL) and ethyl 2-chloro-2-oxo-acetate (14.30 mg, 104.74 μmol, 11.72 μL) and the mixture was stirred at ambient temperature. After completion, the reaction mixture was diluted with chloroform/isopropanol (4:1) and NaHCO₃(aqueous). The separated organic layer was washed with brine, dried over Na₂SO₄, and concentrated. The mixture was purified by silica gel column chromatography using a 0% to 20% methanol in dichloromethane eluent gradient to yield ethyl 2-[4-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]-1-piperidyl]-2-oxo-acetate (37 mg, 55.00 μmol, 58% yield). LCMS (ESI+): 673.2 (M+H).

### Step 2: 2-[4-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]-1-piperidyl]-2-oxo-acetic acid lithium salt

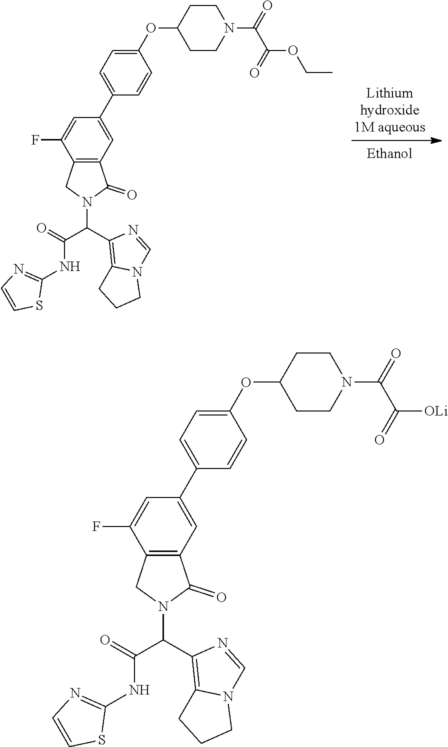

To a solution of ethyl 2-[4-[4-[2-[1-(6,7-dihydro-5H-pyrrolo[1,2-c]imidazol-1-yl)-2-oxo-2-(2-thienylamino)ethyl]-7-fluoro-3-oxo-isoindolin-5-yl]phenoxy]-1-piperidyl]-2-oxo-acetate (37 mg, 55.08 μmol) in Ethanol (0.5 mL) was added Lithium hydroxide (1 M aqueous solution, 61 μmol, 61 μL) and the mixture was stirred at ambient temperature for 2 hours. The reaction mixture was evaporated to dryness under reduced pressure to afford 2-[4-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]-1-piperidyl]-2-oxo-acetic acid lithium salt (35.9 mg, 55.08 μmol, quantitative yield). LCMS (ESI+): 645.2 (M+H).

### Step 3: 2-(6,7-dihydro-5H-pyrrolo[1,2-c]imidazol-1-yl)-2-[6-[4-[[1-[2-[4-[4-[(2,6-dioxo-3-piperidyl)amino]phenyl]-1-piperidyl]-2-oxo-acetyl]-4-piperidyl]oxy]phenyl]-4-fluoro-1-oxo-isoindolin-2-yl]-N-thiazol-2-yl-acetamide

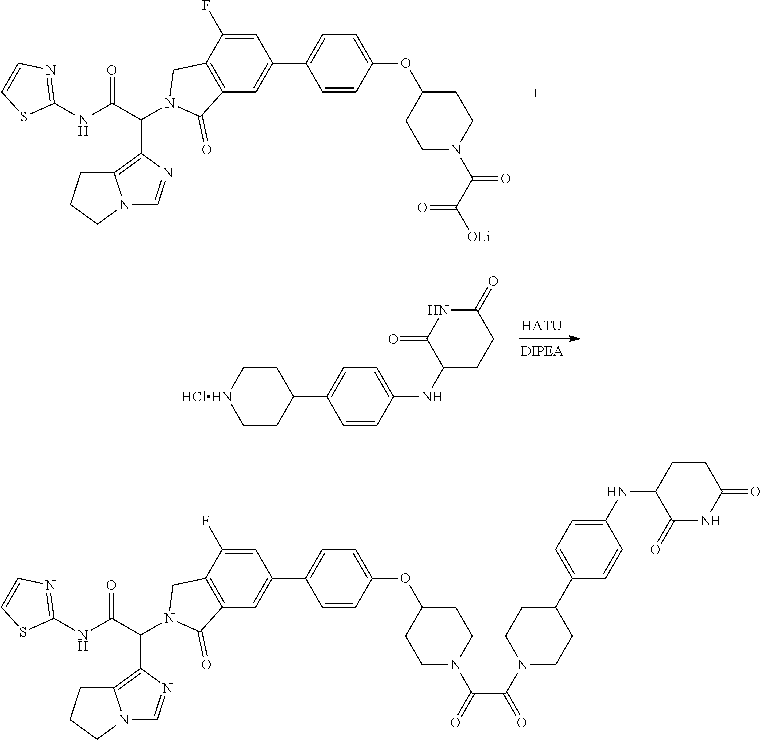

2-[4-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]-1-piperidyl]-2-oxo-acetic acid lithium salt (35.9 mg, 55.08 μmol) and 3-[4-(4-piperidyl)anilino]piperidine-2,6-dione; hydrochloride (21.40 mg, 66.09 μmol) were mixed in DMF (0.5 mL) and the reaction mixture was cooled to 0° C. N,N-Diisopropylethylamine (35.59 mg, 275.39 μmol, 47.97 μL) was added to the reaction mixture, and HATU (27.23 mg, 71.60 μmol) was added, and the reaction mixture was stirred for 1 h at 0° C. The reaction mixture was acidified with 4-5 drops of TFA, and injected directly on a RP C18 column (50 g C18) for purification (5% to 100% ACETONITRILE (+0.1% TFA) in water (+0.1% TFA) over 12 minutes). The pure fractions were neutralized with aqueous NaHCO₃ (ca. 60 mL), extracted twice with isopropanol: chloroform mixture (1:4). The organic layer was dried over Na₂SO₄, filtered, and evaporated under reduced pressure. The residue was purified by silica gel column chromatography (0% to 20% methanol in dichloromethane). The desired fractions were evaporated under reduced pressure, then dissolved in dichloromethane, transferred to an 8 mL vial, and evaporated under reduced pressure. Water (1 mL) and (1 mL) acetonitrile were added, and the mixture was thoroughly sonicated, vortexed and sonicated again. The suspension was frozen and lyophilized to afford Compound 40 (25.7 mg, 27.84 μmol, 50% yield). LCMS (ESI+): 914.3 (M+H), ¹H NMR (400 MHz, DMSO-d₆) δ 12.57 (s, 1H), 10.82 (d, J=2.9 Hz, 1H), 7.86 (s, 1H), 7.85-7.77 (m, 3H), 7.67 (s, 1H), 7.55 (d, J=3.6 Hz, 1H), 7.32 (d, J=3.6 Hz, 1H), 7.18 (d, J=8.4 Hz, 2H), 7.03 (d, J=8.1 Hz, 2H), 6.68 (dd, J=8.7, 2.9 Hz, 2H), 6.22 (s, 1H), 5.75 (dd, J=7.5, 3.6 Hz, 1H), 4.94-4.79 (m, 2H), 4.48 (d, J=12.6 Hz, 1H), 4.37-4.25 (m, 2H), 4.13-3.99 (m, 2H), 3.99-3.84 (m, 1H), 3.64 (t, J=14.8 Hz, 2H), 3.57-3.34 (m, 3H), 3.28 (t, J=13.3 Hz, 1H), 2.92-2.68 (m, 4H), 2.68-2.57 (m, 3H), 2.21-2.01 (m, 3H), 1.99-1.81 (m, 3H), 1.81-1.65 (m, 2H), 1.53 (dd, J=15.6, 7.9 Hz, 2H).

### Example 20. Synthesis of 2-(6,7-dihydro-5H-pyrrolo[1,2-c]imidazol-1-yl)-2-[6-[4-[(3R)-1-[2-[4-[4-[[(3S)-2,6-dioxo-3-piperidyl]amino]phenyl]-1-piperidyl]acetyl]pyrrolidin-3-yl]oxyphenyl]-4-fluoro-1-oxo-isoindolin-2-yl]-N-thiazol-2-yl-acetamide (Compound 41)

### Step 1: tert-butyl 4-(4-(2-(1-(6,7-dihydro-5H-pyrrolo[1,2-c]imidazol-1-yl)-2-ethoxy-2-oxoethyl)-7-fluoro-3-oxoisoindolin-5-yl)phenoxy)piperidine-1-carboxylate

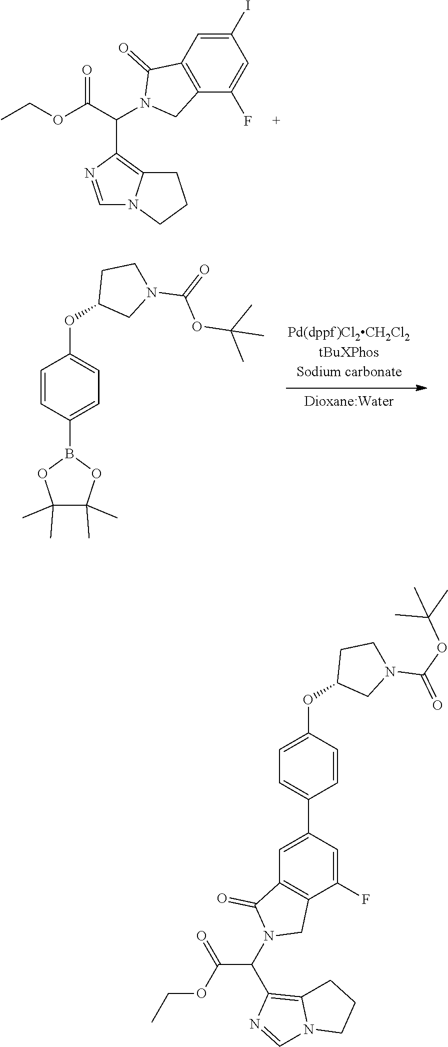 [SMILES: CCOC(=O)C(c1ncn2c1CCC2)N1Cc2c(F)cc(I)cc2C1=O ; CC(C)(C)OC(=O)N1CC[C@@H](Oc2ccc(B3OC(C)(C)C(C)(C)O3)cc2)C1 ; CCOC(=O)C(c1ncn2c1CCC2)N1Cc2c(F)cc(-c3ccc(O[C@@H]4CCN(C(=O)OC(C)(C)C)C4)cc3)cc2C1=O]

tert-Butyl (3R)-3-(4-(2-(1-(6,7-dihydro-5H-pyrrolo[1,2-c]imidazol-1-yl)-2-ethoxy-2-oxoethyl)-7-fluoro-3-oxoisoindolin-5-yl)phenoxy)pyrrolidine-1-carboxylate was prepared from ethyl 2-(6,7-dihydro-5H-pyrrolo[1,2-c]imidazol-1-yl)-2-(4-fluoro-6-iodo-1-oxoisoindolin-2-yl)acetate and tert-butyl (R)-3-(4-(4,4,5,5-tetramethyl-1,3,2-dioxaborolan-2-yl)phenoxy)pyrrolidine-1-carboxylate (Cas #1383793-73-4) in 43% yield using the procedure used in Example 10, step 1. LCMS (ESI+): 605.3 (M+H)

### Step 2: 2-[6-[4-[(3R)-1-tert-butoxycarbonylpyrrolidin-3-yl]oxyphenyl]-4-fluoro-1-oxo-isoindolin-2-yl]-2-(6,7-dihydro-5H-pyrrolo[1,2-c]imidazol-1-yl)acetic acid

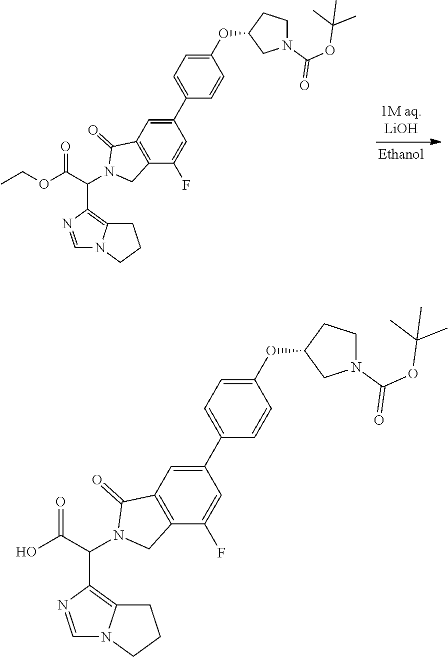 [SMILES: CCOC(=O)C(c1ncn2c1CCC2)N1Cc2c(F)cc(-c3ccc(O[C@@H]4CCN(C(=O)OC(C)(C)C)C4)cc3)cc2C1=O ; CC(C)(C)OC(=O)N1CC[C@@H](Oc2ccc(-c3cc(F)c4c(c3)C(=O)N(C(C(=O)O)c3ncn5c3CCC5)C4)cc2)C1]

To a solution of tert-butyl (3R)-3-[4-[2-[1-(6,7-dihydro-5H-pyrrolo[1,2-c]imidazol-1-yl)-2-ethoxy-2-oxo-ethyl]-7-fluoro-3-oxo-isoindolin-5-yl]phenoxy]pyrrolidine-1-carboxylate (600 mg, 992.28 μmol) in THF (3 mL) and Methanol (3 mL) and Water (3 mL) was added Lithium hydroxide monohydrate (41.64 mg, 992.28 μmol) at 0° C. The reaction mixture was stirred at room temperature for 3 h, then the reaction mixture was concentrated. The crude residue was dissolved in 5 ml water and acidified by using KHSO₄ salt (pH 1-2). The solution was filtered to get solid compound 2-[6-[4-[(3R)-1-tert-butoxycarbonylpyrrolidin-3-yl]oxyphenyl]-4-fluoro-1-oxo-isoindolin-2-yl]-2-(6,7-dihydro-5H-pyrrolo[1,2-c]imidazol-1-yl)acetic acid (450 mg, 522 μmol, 53% yield). LCMS (ESI+): 577.0 (M+H).

### Step 3: (3R)-3-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]pyrrolidine-1-carboxylate

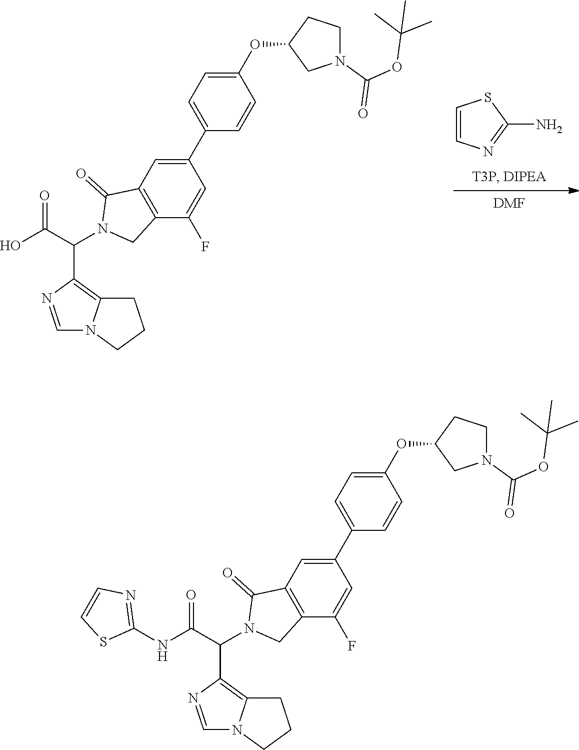

To a solution of 2-[6-[4-[(3R)-1-tert-butoxycarbonylpyrrolidin-3-yl]oxyphenyl]-4-fluoro-1-oxo-isoindolin-2-yl]-2-(6,7-dihydro-5H-pyrrolo[1,2-c]imidazol-1-yl)acetic acid (200 mg, 346.85 μmol) in DMF (2 mL) was added N,N-diisopropyl ethyl amine (224.14 mg, 1.73 mmol, 302.08 μL) and propylphosphonic anhydride, 50% solution in ethyl acetate (220.72 mg, 693.70 μmol) at 0° C. After 15 min, thiazol-2-amine (34.73 mg, 346.85 μmol) was added and the mixture was stirred at 50° C. for 16 hours. Water was added to the reaction mixture to afford precipitation. The precipitate was collected by filtration, then was dissolved in dichloromethane and the solution was concentrated. The crude product was purified by flash column chromatography, product eluted in 3% methanol/dichloromethane. The appropriate fractions were combined and concentrated to give tert-butyl (3R)-3-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]pyrrolidine-1-carboxylate (110 mg, 111.9 μmol, 32% yield). LMCS (ESI+): 659.2 (M+H).

### Step 4: 2-(6,7-dihydro-5H-pyrrolo[1,2-c]imidazol-1-yl)-2-[4-fluoro-1-oxo-6-[4-[(3R)-pyrrolidin-3-yl]oxyphenyl]isoindolin-2-yl]-N-thiazol-2-yl-acetamide hydrochloride

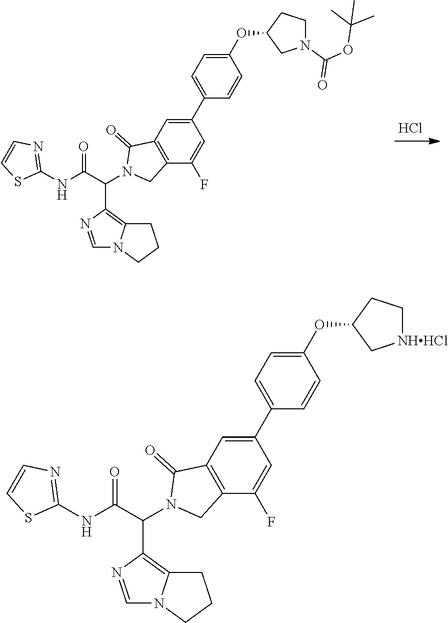

To a solution of tert-butyl (3R)-3-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenoxy]pyrrolidine-1-carboxylate (150 mg, 227.71 μmol) in dichloromethane (1.5 mL) was added Hydrogen chloride solution (4.0M in dioxane, 426 μL, 1.71 mmol) at 0° C. After 2 h the reaction mixture was concentrated under reduced pressure and the solid residue was washed with diethyl ether to afford 2-(6,7-dihydro-5H-pyrrolo[1,2-c]imidazol-1-yl)-2-[4-fluoro-1-oxo-6-[4-[(3R)-pyrrolidin-3-yl]oxyphenyl]isoindolin-2-yl]-N-thiazol-2-yl-acetamide hydrochloride (140 mg, 174.1 μmol, 76.5% yield). LCMS m/z 559.2 (M+H)

### Step 5: 2-(6,7-dihydro-5H-pyrrolo[1,2-c]imidazol-1-yl)-2-[6-[4-[(3R)-1-[2-[4-[4-[[(3S)-2,6-dioxo-3-piperidyl]amino]phenyl]-1-piperidyl]acetyl]pyrrolidin-3-yl]oxyphenyl]-4-fluoro-1-oxo-isoindolin-2-yl]-N-thiazol-2-yl-acetamide

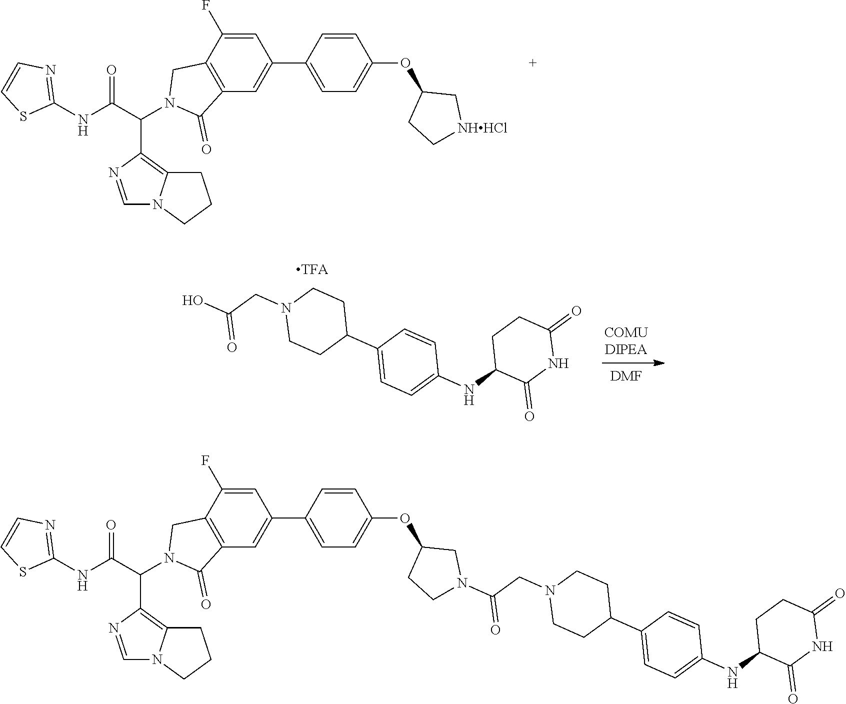

To a solution of 2-[4-[4-[[(3S)-2,6-dioxo-3-piperidyl]amino]phenyl]-1-piperidyl]acetic acid (63.84 mg, 138.97 μmol) in DMF (1 mL) was added N,N-diisopropyl ethyl amine (119.45 mg, 924.24 μmol, 160.99 μL) and COMU (118.75 mg, 277.27 μmol) at 0° C. After 15 min, 2-(6,7-dihydro-5H-pyrrolo[1,2-c]imidazol-1-yl)-2-[4-fluoro-1-oxo-6-[4-[(3R)-pyrrolidin-3-yl]oxyphenyl]isoindolin-2-yl]-N-thiazol-2-yl-acetamide hydrochloride (110 mg, 184.85 μmol) was added. After stirring for 1 hour, 5 ml of water added to the reaction mixture and a solid precipitate was formed. The solid was collected by filtration, then dissolved in dichloromethane and the solution was concentrated. The crude was purified by reverse phase C-18 chromatography (0-100% of 0.1% ammonium acetate in water and Acetonitrile). Fractions were lyophilized to get afford Compound 41 as a white solid. LCMS (ESI+): 887.0 (M+H), 1H-NMR (400 MHz, DMSO-d₆): 12.53 (s, 1H), 10.77 (s, 1H), 7.80-7.77 (m, 4H), 7.64 (s, 1H), 7.55 (s, 1H), 7.26 (s, 1H), 7.11-7.02 (m, 2H), 6.97-6.95 (m, 1H), 6.91-6.87 (m, 1H), 6.62 (d, J=8.4 Hz, 1H), 6.56 (s, 1H), 6.15 (s, 1H), 5.72-5.65 (m, 1H), 5.23-5.13 (m, 1H), 4.82 (d, J=17.6 Hz, 1H), 4.44-4.24 (m, 2H), 4.04-3.92 (m, 2H), 3.90-3.61 (m, 3H), 3.61-3.45 (m, 2H), 3.61-3.45 (m, 4H), 3.11-2.88 (m, 2H), 2.90-2.85 (m, 1H), 2.71-2.67 (m, 2H), 2.65-2.58 (m, 2H), 2.12-2.42 (m, 4H), 1.85-1.78 (m, 1H), 1.72-1.64 (m, 2H)

### Example 21. Synthesis of (2RS)-2-[6-[4-[4-[2-[4-[4-[[(3RS)-2,6-Dioxo-3-piperidyl]amino]phenyl]-1-piperidyl]acetyl]piperazin-1-yl]phenyl]-4-fluoro-1-oxo-isoindolin-2-yl]-2-[(6R)-6-fluoro-6,7-dihydro-5H-pyrrolo[1,2-c]imidazol-1-yl]-N-thiazol-2-yl-acetamide (Compound 43)

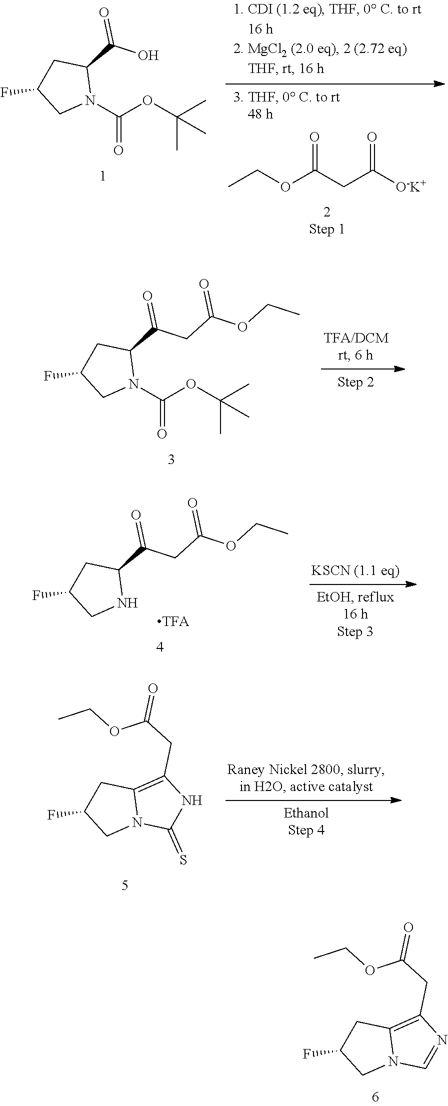

### Step 1: Tert-butyl (2S,4R)-2-(3-ethoxy-3-oxopropanoyl)-4-fluoropyrrolidine-1-carboxylate

Tert-butyl (2S,4R)-2-(3-ethoxy-3-oxopropanoyl)-4-fluoropyrrolidine-1-carboxylate was obtained in quantitative yield using a procedure similar to that used for Intermediate Ethyl 2-amino-2-(6,7-dihydro-5H-pyrrolo[1,2-c]imidazol-1-yl)acetate dihydrochloride, Step 1, using (2S,4R)-1-(tert-butoxycarbonyl)-4-fluoropyrrolidine-2-carboxylic acid (CAS #203866-14-2) instead of tert-butyl (2S,4R)-2-(3-ethoxy-3-oxopropanoyl)-pyrrolidine-1-carboxylate. LCMS (ESI+): 304.1 (M+H)

### Step 2: Ethyl 3-((2S,4R)-4-fluoropyrrolidin-2-yl)-3-oxopropanoate, trifluoroacetic acid salt

Tert-butyl (2S,4R)-2-(3-ethoxy-3-oxopropanoyl)-4-fluoropyrrolidine-1-carboxylate (11 grams, 36 mmol) was dissolved in dichloromethane (150 mL) and trifluoroacetic acid (50 mL) was added. The reaction mixture was stirred for 2 hours at 22° C., and the volatiles were evaporated to afford ethyl 3-((2S,4R)-4-fluoropyrrolidin-2-yl)-3-oxopropanoate, trifluoroacetic acid salt (7.36 g, 36 mmol, quantitative yield). LCMS (ESI+): 204.1 (M+H).

### Step 3: Ethyl (R)-2-(6-fluoro-3-thioxo-2,5,6,7-tetrahydro-3H-pyrrolo[1,2-c]imidazol-1-yl)acetate

Ethyl (R)-2-(6-fluoro-3-thioxo-2,5,6,7-tetrahydro-3H-pyrrolo[1,2-c]imidazol-1-yl)acetate was obtained in 88% yield from ethyl 3-((2S,4R)-4-fluoropyrrolidin-2-yl)-3-oxopropanoate, trifluoroacetic acid salt using a procedure similar to that used for Intermediate Ethyl 2-amino-2-(6,7-dihydro-5H-pyrrolo[1,2-c]imidazol-1-yl)acetate dihydrochloride, Step 3. LCMS (ESI+): 245.1 (M+H)

### Step 4: Ethyl (R)-2-(6-fluoro-6,7-dihydro-5H-pyrrolo[1,2-c]imidazol-1-yl)acetate

Ethyl (R)-2-(6-fluoro-6,7-dihydro-5H-pyrrolo[1,2-c]imidazol-1-yl)acetate was obtained in 47% yield from using a procedure similar to that used for the synthesis of Intermediate Ethyl 2-amino-2-(6,7-dihydro-5H-pyrrolo[1,2-c]imidazol-1-yl)acetate dihydrochloride, Step 4. LCMS (ESI+) m/z=213 [M+H⁺], 41-NMR (400 MHz, CDCl₃: 7.51 (br. s, 1H), 5.79 (d, J=51 Hz, 1H), 4.30-4.09 (m, 4H), 3.59 (br, s, 2H), 3.25-3.02 (m, 2H), 1.28 (t, J=6.7 Hz, 3H).

### Step 5: ethyl 2-[(6R)-6-fluoro-6,7-dihydro-5H-pyrrolo[1,2-c]imidazol-1-yl]-2-hydroxyimino-acetate

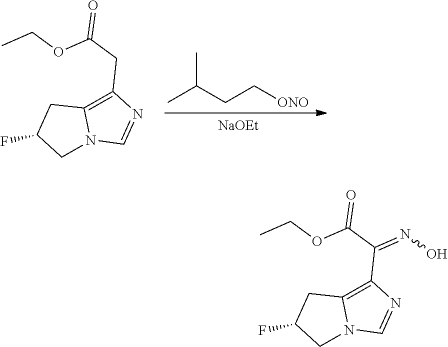

Ethyl 2-[(6R)-6-fluoro-6,7-dihydro-5H-pyrrolo[1,2-c]imidazol-1-yl]-2-hydroxyimino-acetate was obtained in 77.5% yield from ethyl (R)-2-(6-fluoro-6,7-dihydro-5H-pyrrolo[1,2-c]imidazol-1-yl)acetate using a procedure similar to that used for Intermediate Ethyl 2-amino-2-(6,7-dihydro-5H-pyrrolo[1,2-c]imidazol-1-yl)acetate dihydrochloride, Step 5. LCMS (ESI+): 242.1 (M+H⁺)

### Step 6: ethyl 2-amino-2-[(6R)-6-fluoro-6,7-dihydro-5H-pyrrolo[1,2-c]imidazol-1-yl]acetate

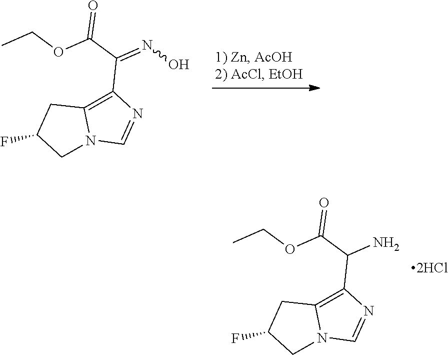 [SMILES: CCOC(=O)C(=NO)c1ncn2c1C[C@@H](F)C2 ; CCOC(=O)C(N)c1ncn2c1C[C@@H](F)C2]

Ethyl 2-amino-2-[(6R)-6-fluoro-6,7-dihydro-5H-pyrrolo[1,2-c]imidazol-1-yl]acetate dihydrochloride was obtained in 29% yield from ethyl 2-[(6R)-6-fluoro-6,7-dihydro-5H-pyrrolo[1,2-c]imidazol-1-yl]-2-hydroxyimino-acetate using a procedure similar as the one used for Intermediate Ethyl 2-amino-2-(6,7-dihydro-5H-pyrrolo[1,2-c]imidazol-1-yl)acetate dihydrochloride, Step 6. LCMS (ESI+): 228.1 (M+H).

### Step 7: 2-[(6R)-6-fluoro-6,7-dihydro-5H-pyrrolo[1,2-c]imidazol-1-yl]-2-(4-fluoro-6-iodo-1-oxo-isoindolin-2-yl)acetate

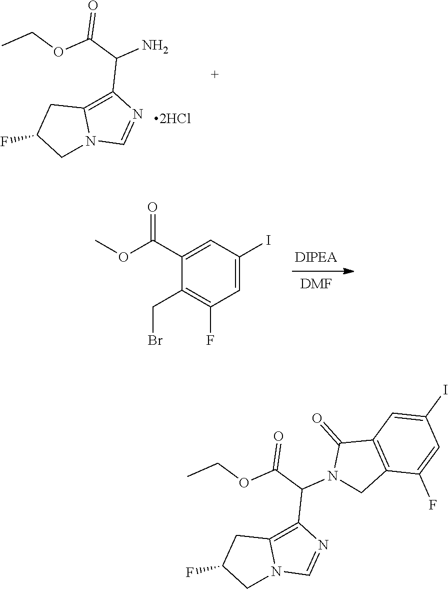

Ethyl 2-amino-2-[(6R)-6-fluoro-6,7-dihydro-5H-pyrrolo[1,2-c]imidazol-1-yl]acetate; dihydrochloride (3.9 g, 4.02 mmol) was dissolved in DMF (10 mL). Methyl 2-(bromomethyl)-3-fluoro-5-iodo-benzoate (1.20 g, 3.22 mmol) was added, followed by N-ethyl-N-isopropyl-propan-2-amine (2.08 g, 16.08 mmol, 2.80 mL). The reaction mixture was stirred at rt for 30 min. The reaction mixture was heated at 80° C. for 4 h. The reaction mixture was partitioned between ethyl acetate and sodium bicarbonate (aqueous, aqueous). The organic layer was isolated and washed with brine, dried with sodium sulfate, filtered and evaporated under reduced pressure. The crude residue was purified by silica gel chromatography (40 g column, 0% to 25% methanol in ethyl acetate). Pure fractions were evaporated to afford ethyl 2-[(6R)-6-fluoro-6,7-dihydro-5H-pyrrolo[1,2-c]imidazol-1-yl]-2-(4-fluoro-6-iodo-1-oxo-isoindolin-2-yl)acetate (951 mg, 1.95 mmol, 48.55% yield). LCMS: 1.253 min., MS (ESI+): 488 (M+H)

### Step 8: tert-butyl 4-[4-[2-[2-ethoxy-1-[(6R)-6-fluoro-6,7-dihydro-5H-pyrrolo[1,2-c]imidazol-1-yl]-2-oxo-ethyl]-7-fluoro-3-oxo-isoindolin-5-yl]phenyl]piperazine-1-carboxylate

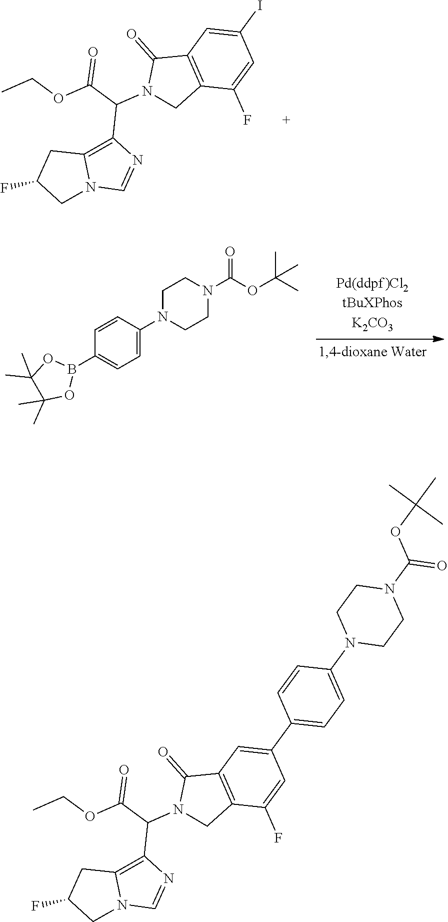

Ethyl 2-[(6R)-6-fluoro-6,7-dihydro-5H-pyrrolo[1,2-c]imidazol-1-yl]-2-(4-fluoro-6-iodo-1-oxo-isoindolin-2-yl)acetate (951 mg, 1.95 mmol) and tert-butyl 4-[4-(4,4,5,5-tetramethyl-1,3,2-dioxaborolan-2-yl)phenyl]piperazine-1-carboxylate (833.70 mg, 2.15 mmol) were mixed in water (2.5 mL) and 1,4-dioxane (7.5 mL). Pd(dppf)Cl₂ (99.97 mg, 136.63 μmol) and Potassium carbonate (269.75 mg, 1.95 mmol, 117.80 μL) were added, and the reaction mixture was degassed with nitrogen under sonication for 15 minutes. The reaction mixture was heated at 80° C. for 4 hours. The reaction mixture was partitioned between ethyl acetate and sodium bicarbonate (aqueous, aqueous). The organic layer was washed with brine, dried with sodium sulfate, filtered and evaporated under reduced pressure. The crude residue was purified by silica gel chromatography (40 g column, 0% to 20% methanol in ethyl acetate). Pure fractions were evaporated to afford tert-butyl 4-[4-[2-[2-ethoxy-1-[(6R)-6-fluoro-6,7-dihydro-5H-pyrrolo[1,2-c]imidazol-1-yl]-2-oxo-ethyl]-7-fluoro-3-oxo-isoindolin-5-yl]phenyl]piperazine-1-carboxylate (436 mg, 701.33 μmol, 35.93% yield). LCMS (ESI+): 622.2 (M+H)/522 (M-Boc+H)

### Step 9: [2-[6-[4-(4-tert-butoxycarbonylpiperazin-1-yl)phenyl]-4-fluoro-1-oxo-isoindolin-2-yl]-2-[(6R)-6-fluoro-6,7-dihydro-5H-pyrrolo[1,2-c]imidazol-1-yl]acetyl]oxylithium

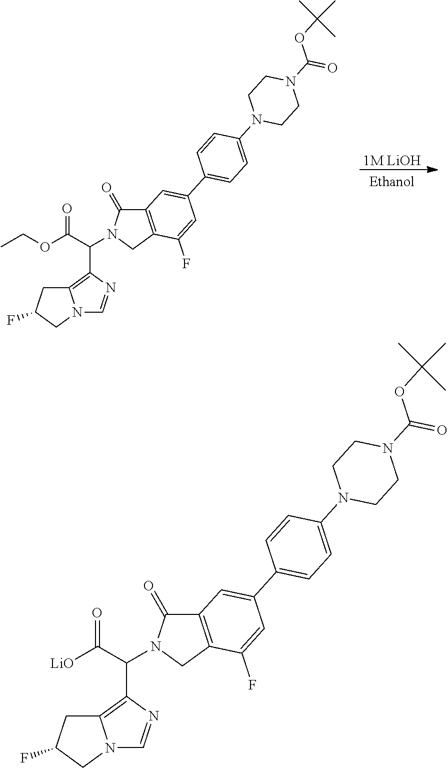

Tert-butyl 4-[4[2-[2-ethoxy-1-[(6R)-6-fluoro-6,7-dihydro-5H-pyrrolo[1,2-c]imidazol-1-yl]-2-oxo-ethyl]-7-fluoro-3-oxo-isoindolin-5-yl]phenyl]piperazine-1-carboxylate (435 mg, 699.73 μmol) was dissolved in ethanol (5 mL), cooled to 0° C. and lithium hydroxide, 1M (1 M, 699.73 μL) was added. The reaction mixture was stirred for 3 hours. The crude residue was dissolved in dichloromethane with 0.5 mL of benzene and evaporated under reduced pressure, then submitted to high vacuum to afford [2-[6-[4-(4-tert-butoxycarbonylpiperazin-1-yl)phenyl]-4-fluoro-1-oxo-isoindolin-2-yl]-2-[(6R)-6-fluoro-6,7-dihydro-5H-pyrrolo[1,2-c]imidazol-1-yl]acetyl]oxylithium (410 mg, 683.84 μmol, 97.73% yield). LCMS (ESI+): 594.2 (M+H, free acid).

### Step 10: tert-butyl 4-[4-[7-fluoro-2-[1-[(6R)-6-fluoro-6,7-dihydro-5H-pyrrolo[1,2-c]imidazol-1-yl]-2-oxo-2-(thiazol-2-ylamino)ethyl]-3-oxo-isoindolin-5-yl]phenyl]piperazine-1-carboxylate

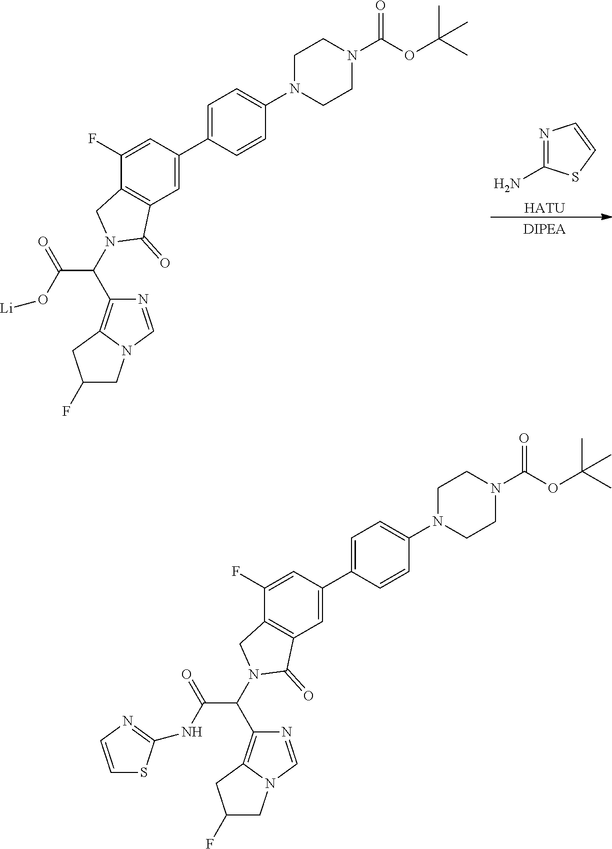

[2-[6-[4-(4-tert-butoxycarbonylpiperazin-1-yl)phenyl]-4-fluoro-1-oxo-isoindolin-2-yl]-2-[(6R)-6-fluoro-6,7-dihydro-5H-pyrrolo[1,2-c]imidazol-1-yl]acetyl]oxylithium (410 mg, 683.84 μmol) and thiazol-2-amine (82.18 mg, 820.61 μmol) were mixed in DMF and cooled to 0° C. N,N-diisopropylethylamine (265.15 mg, 2.05 mmol, 357.34 μL) was added to the reaction mixture, and HATU (312.02 mg, 820.61 μmol) was added, and the reaction mixture was stirred for 4 hours. The reaction mixture was partitioned between ethyl acetate and sodium bicarbonate (aqueous, aqueous). The organic layer was washed with brine, dried with sodium sulfate, filtered and evaporated under reduced pressure. The crude residue was purified by silica gel chromatography (24 g column, 0% to 20% methanol in dichloromethane). Pure fractions were evaporated to afford tert-butyl 4-[4-[7-fluoro-2-[1-[(6R)-6-fluoro-6,7-dihydro-5H-pyrrolo[1,2-c]imidazol-1-yl]-2-oxo-2-(thiazol-2-ylamino)ethyl]-3-oxo-isoindolin-5-yl]phenyl]piperazine-1-carboxylate (315 mg, 419.54 μmol, 61.35% yield). LCMS (ESI+): 676.2 (M+H).

### Step 11: 2-[(6R)-6-fluoro-6,7-dihydro-5H-pyrrolo[1,2-c]imidazol-1-yl]-2-[4-fluoro-1-oxo-6-(4-piperazin-1-ylphenyl)isoindolin-2-yl]-N-thiazol-2-yl-acetamide; hydrochloride

### Step 11: (2RS)-2-[(6R)-6-Fluoro-6,7-dihydro-5H-pyrrolo[1,2-c]imidazol-1-yl]-2-[4-fluoro-1-oxo-6-(4-piperazin-1-ylphenyl)isoindolin-2-yl]-N-thiazol-2-yl-acetamide hydrochloride salt

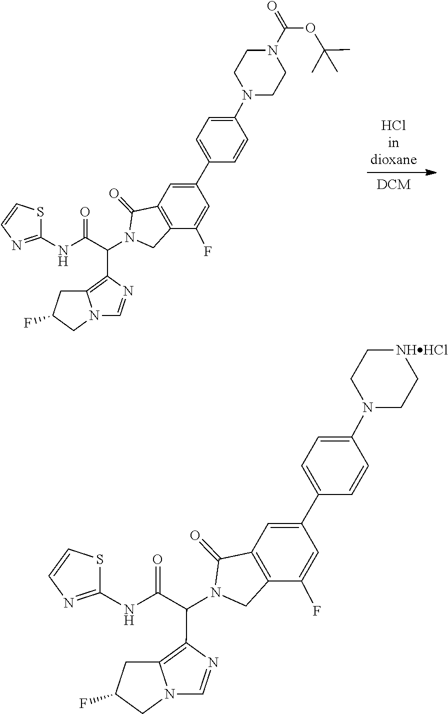

To a solution of tert-butyl 4-[4-[7-fluoro-2-[(1RS)-1-[(6R)-6-fluoro-6,7-dihydro-5H-pyrrolo[1,2-c]imidazol-1-yl]-2-oxo-2-(thiazol-2-ylamino)ethyl]-3-oxo-isoindolin-5-yl]phenyl]piperazine-1-carboxylate (25 mg, 37 μmol) in dichloromethane (0.5 ml) was added HCl 4M in dioxane (46.2 μl, 185 μmol, Eq: 5). The reaction mixture was stirred at room temperature for 3 hours. The reaction mixture was concentrated to dryness to afford crude (2RS)-2-[(6R)-6-fluoro-6,7-dihydro-5H-pyrrolo[1,2-c]imidazol-1-yl]-2-[4-fluoro-1-oxo-6-(4-piperazin-1-ylphenyl)isoindolin-2-yl]-N-thiazol-2-yl-acetamide hydrochloride salt (22.6 mg, 36.9 μmol, 99.7%) as an off-white solid. MS: m/e=576.4 ([M+H]⁺).

### Step 12:2-(4-(4-((2,6-Dioxopiperidin-3-yl)amino)phenyl)piperidin-1-yl)acetic acid hydrochloride

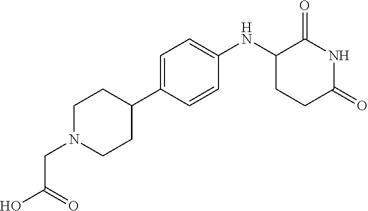 [SMILES: O=C(O)CN1CCC(c2ccc(NC3CCC(=O)NC3=O)cc2)CC1]

To a solution of tert-butyl 2-(4-(4-((2,6-dioxopiperidin-3-yl)amino)phenyl)piperidin-1-yl)acetate (543 mg, 1.35 mmol, Eq: 1) in ethyl acetate (8 ml) was added 4 M hydrogen chloride solution in 1,4-dioxane (6.3 g, 6 ml, 24 mmol, Eq: 17.7) at room temperature and stirring was continued over the weekend. The product was collected by filtration, washed with ethyl acetate and dried over high vacuo to afford 2-(4-(4-((2,6-dioxopiperidin-3-yl)amino)phenyl)piperidin-1-yl)acetic acid hydrochloride (537 mg, 1.27 mmol, 93.6% yield) as light red solid. MS: m/e=346.2 ([M+H]⁺).

### Step 13: (2RS)-2-[6-[4-[4-[2-[4-[4-[[(3RS)-2,6-Dioxo-3-piperidyl]amino]phenyl]-1-piperidyl]acetyl]piperazin-1-yl]phenyl]-4-fluoro-1-oxo-isoindolin-2-yl]-2-[(6R)-6-fluoro-6,7-dihydro-5H-pyrrolo[1,2-c]imidazol-1-yl]-N-thiazol-2-yl-acetamide

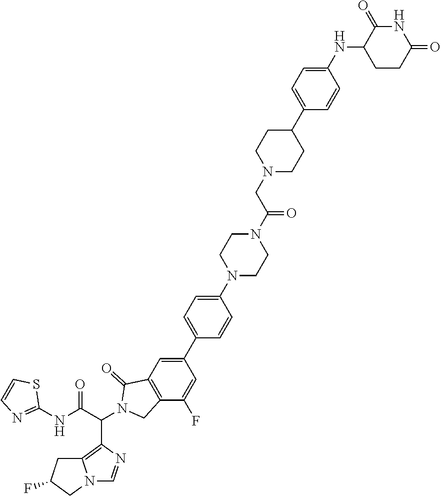 [SMILES: O=C1CCC(Nc2ccc(C3CCN(CC(=O)N4CCN(c5ccc(-c6cc(F)c7c(c6)C(=O)N(C(C(=O)Nc6nccs6)c6ncn8c6C[C@@H](F)C8)C7)cc5)CC4)CC3)cc2)C(=O)N1]

The title compound, Compound 43, was obtained as a light yellow solid, MS: m/e=903.7 ([M+H]⁺), using chemistry similar to that described in Example 1.

### Example 22. Synthesis of 2-[6-[4-[4-[2-[4-[4-[[(3S)-2,6-dioxo-3-piperidyl]amino]-2-fluoro-phenyl]-1-piperidyl]acetyl]piperazin-1-yl]phenyl]-4-fluoro-1-oxo-isoindolin-2-yl]-2-[(6R)-6-fluoro-6,7-dihydro-5H-pyrrolo[1,2-c]imidazol-1-yl]-N-thiazol-2-yl-acetamide (Compound 44)

### Step 1: 2-[(6R)-6-fluoro-6,7-dihydro-5H-pyrrolo[1,2-c]imidazol-1-yl]-2-[4-fluoro-1-oxo-6-(4-piperazin-1-ylphenyl)isoindolin-2-yl]-N-thiazol-2-yl-acetamide hydrochloride

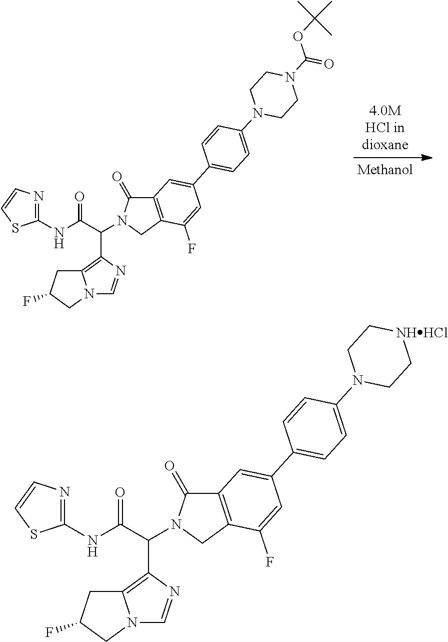

tert-Butyl 4-[4-[7-fluoro-2-[1-[(6R)-6-fluoro-6,7-dihydro-5H-pyrrolo[1,2-c]imidazol-1-yl]-2-oxo-2-(thiazol-2-ylamino)ethyl]-3-oxo-isoindolin-5-yl]phenyl]piperazine-1-carboxylate (315 mg, 466.15 μmol) was dissolved in Methanol (3 mL) and Hydrogen chloride solution 4.0M in dioxane (4 M, 5.6 mmol, 1.40 mL) was added. The reaction mixture was heated at 40° C. for 4 hours, and the reaction was complete. The volatiles were evaporated under reduce pressure. The material was submitted to high vacuum, frozen to −78° C. and thawed to afford 2-[(6R)-6-fluoro-6,7-dihydro-5H-pyrrolo[1,2-c]imidazol-1-yl]-2-[4-fluoro-1-oxo-6-(4-piperazin-1-ylphenyl)isoindolin-2-yl]-N-thiazol-2-yl-acetamide hydrochloride (237 mg, 387.20 μmol, 83.06% yield). LCMS (ESI+): 576.2 (M+H)

### Step 2: 2-[6-[4-[4-[2-[4-[4-[[(3S)-2,6-dioxo-3-piperidyl]amino]-2-fluoro-phenyl]-1-piperidyl]acetyl]piperazin-1-yl]phenyl]-4-fluoro-1-oxo-isoindolin-2-yl]-2-[(6R)-6-fluoro-6,7-dihydro-5H-pyrrolo[1,2-c]imidazol-1-yl]-N-thiazol-2-yl-acetamide

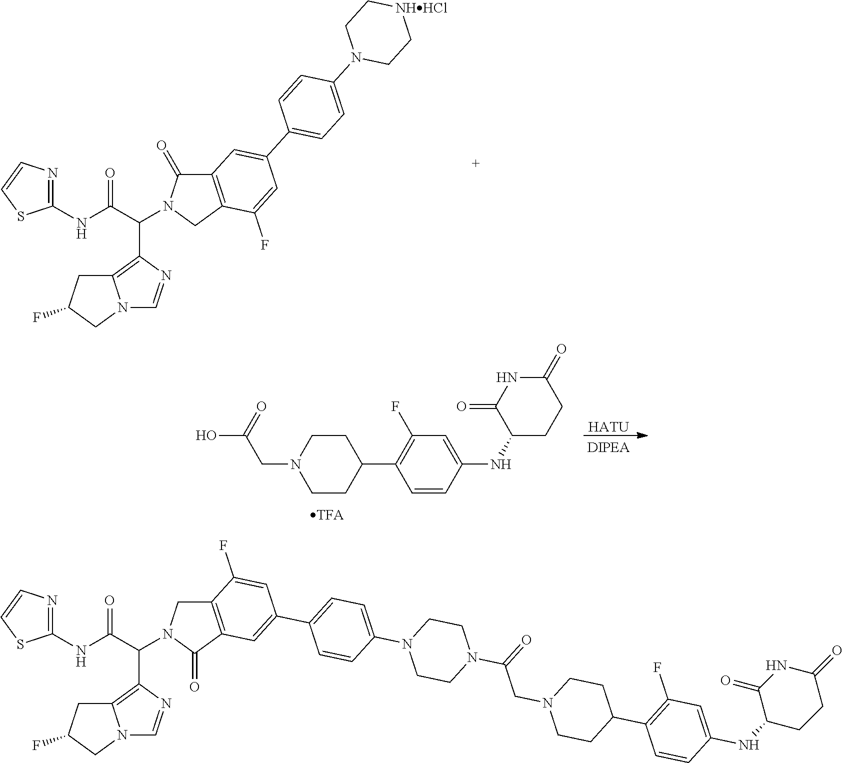

2-[(6R)-6-Fluoro-6,7-dihydro-5H-pyrrolo[1,2-c]imidazol-1-yl]-2-[4-fluoro-1-oxo-6-(4-piperazin-1-ylphenyl)isoindolin-2-yl]-N-thiazol-2-yl-acetamide; hydrochloride (63 mg, 102.93 μmol) and 2-[4-[4-[[(3S)-2,6-dioxo-3-piperidyl]amino]-2-fluoro-phenyl]-1-piperidyl]acetic acid, trifluoroacetic acid (51.59 mg, 108.07 μmol) were mixed in DMAc (0.6 mL) and cooled to 0° C. N,N-diisopropylethylamine (66.51 mg, 514.63 μmol, 89.64 μL) was added to the reaction mixture, and HATU (50.88 mg, 133.80 μmol) was added, and the reaction mixture was stirred while warming for 4 hours. The mixture was injected on a 50 g C18 column, and purified using a 0% to 100% Acetonitrile in water+0.1% TFA water elution gradient. The desired fractions were pooled and partitioned between ethyl acetate and sodium bicarbonate (aqueous, aqueous). The organic layer was washed with brine, dried with sodium sulfate, filtered and evaporated under reduced pressure. The residue was purified by silica gel chromatography (24 g column, 0% to 20% methanol in ethyl acetate). Pure fractions were evaporated; the solid material was dissolved in acetonitrile:water (1:1), frozen and lyophilized to afford Compound 44 (22 mg, 23.41 μmol, 22.74% yield). LCMS (ESI+): 921.2 (M+H). ¹H NMR (400 MHz, DMSO-d₆) δ 12.56 (d, J=6.7 Hz, 1H), 10.77 (s, 1H), 7.89-7.72 (m, 2H), 7.68 (d, J=4.8 Hz, 2H), 7.49 (dd, J=3.6, 2.0 Hz, 1H), 7.26 (dd, J=3.6, 1.8 Hz, 1H), 7.08 (d, J=8.5 Hz, 2H), 6.98 (t, J=8.8 Hz, 1H), 6.56-6.31 (m, 2H), 6.17 (d, J=5.1 Hz, 1H), 5.99 (d, J=7.7 Hz, 1H), 5.82 (d, J=51.1 Hz, 1H), 4.82 (dd, J=17.6, 6.0 Hz, 1H), 4.45-4.16 (m, 4H), 4.08 (q, J=5.3 Hz, 1H), 3.75 (s, 2H), 3.62 (s, 2H), 3.26-3.18 (m, 2H), 3.19-3.09 (m, 3H), 3.01-2.83 (m, 2H), 2.82-2.52 (m, 2H), 2.26-2.01 (m, 4H), 1.85 (qd, J=12.1, 4.6 Hz, 1H), 1.65 (m, 4H).

### Example 23. Synthesis of 2-[6-[4-[4-[2-[4-[3-(2,4-dioxohexahydropyrimidin-1-yl)-1-methyl-indazol-6-yl]-1-piperidyl]acetyl]piperazin-1-yl]phenyl]-4-fluoro-1-oxo-isoindolin-2-yl]-2-[(6R)-6-fluoro-6,7-dihydro-5H-pyrrolo[1,2-c]imidazol-1-yl]-N-thiazol-2-yl-acetamide (Compound 45)

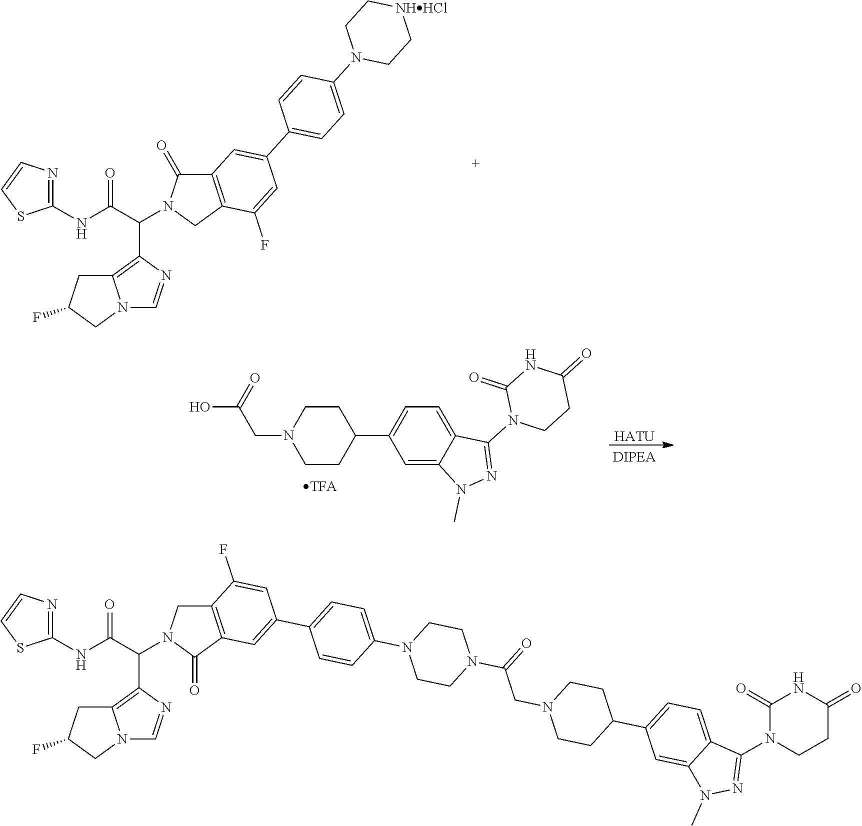

2-[(6R)-6-Fluoro-6,7-dihydro-5H-pyrrolo[1,2-c]imidazol-1-yl]-2-[4-fluoro-1-oxo-6-(4-piperazin-1-ylphenyl)isoindolin-2-yl]-N-thiazol-2-yl-acetamide hydrochloride (120 mg, 196.05 μmol) and 2-[4-[3-(2,4-dioxohexahydropyrimidin-1-yl)-1-methyl-3a,7a-dihydroindazol-6-yl]-1-piperidyl]acetic acid, trifluoroacetic acid salt (83.10 mg, 196.05 μmol) were mixed in DMF (1.2 mL). The reaction mixture was cooled to 0° C. N,N-diisopropylethylamine (170.74 μL, 980.24 μmol) was added to the reaction mixture, and HATU (96.91 mg, 254.86 μmol) was added. The reaction mixture was stirred while warming for 4 h. The mixture was injected on a 50 g C18 column and purified using a 0% to 100% acetonitrile (+0.1% TFA) in water (+0.1% TFA) elution gradient. The desired fractions were pooled and partitioned between ethyl acetate and aqueous saturated sodium bicarbonate. The organic layer was washed with brine, dried with sodium sulfate, filtered and evaporated under reduced pressure. The crude residue was purified by silica gel chromatography (24 g column, 0% to 20% methanol in ethyl acetate). The pure fractions were evaporated to afford Compound 45 (49 mg, 50.92 μmol, 25.97% yield). LCMS (ESI+): 943.3 (M+H⁺), LCMS (ESI−): 941.1 (M−H), ¹H NMR (400 MHz, DMSO-d₆) δ 12.55 (s, 1H), 10.53 (s, 1H), 7.99-7.61 (m, 5H), 7.56 (d, J=8.5 Hz, 1H), 7.51-7.45 (m, 1H), 7.44 (s, 1H), 7.22 (s, 1H), 7.09 (d, J=8.8 Hz, 2H), 7.04 (dd, J=8.6, 1.3 Hz, 1H), 6.15 (s, 1H), 5.95-5.66 (m, 1H), 4.86 (d, J=17.6 Hz, 1H), 4.47-4.07 (m, 3H), 3.97 (s, 3H), 3.90 (t, J=6.7 Hz, 2H), 3.78 (s, 2H), 3.64 (s, 2H), 3.29-3.08 (m, 4H), 3.13-2.84 (m, 3H), 2.75 (t, J=6.7 Hz, 2H), 2.72-2.55 (m, 1H), 2.18 (t, J=10.9 Hz, 2H), 1.97-1.55 (m, 4H).

### Example 24. Synthesis of 2-(6,7-dihydro-5H-pyrrolo[1,2-c]imidazol-1-yl)-2-[6-[4-[2-[2-[4-[4-[(2,6-dioxo-3-piperidyl)amino]phenyl]-1-piperidyl]acetyl]-2,6-diazaspiro[3.3]heptan-6-yl]phenyl]-4-fluoro-1-oxo-isoindolin-2-yl]-N-thiazol-2-yl-acetamide (Compound 47)

### Step 1: tert-butyl 6-(4-bromophenyl)-2,6-diazaspiro[3.3]heptane-2-carboxylate

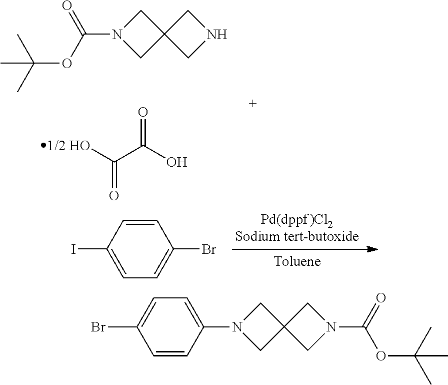

1-Bromo-4-iodo-benzene (9.06 g, 32.01 mmol) and tert-butyl 2,6-diazaspiro[3.3]heptane-2-carboxylate hemioxalate (6.49 g, 13.34 mmol) were suspended in toluene (48 mL), and the reaction mixture was degassed with a nitrogen stream. Sodium tert-butoxide (12.82 g, 133.39 mmol) was added, and the reaction mixture was sonicated, under a nitrogen atmosphere, until the solids were mostly dissolved and the solution was homogeneous. 1,1′-Bis(Diphenylphosphino)ferrocenepalladium (II) dichloride (1.95 g, 2.67 mmol) was added, and the reaction mixture was heated under nitrogen at 90° C. for 16 hours. The solution was filtered through celite, washing with ethyl acetate and the filtrate was evaporated under reduced pressure. The crude residue was purified by silica gel chromatography using a 0% to 50% Ethyl acetate in hexanes eluent gradient to afford tert-butyl 6-(4-bromophenyl)-2,6-diazaspiro[3.3]heptane-2-carboxylate (6.25 g, 17.69 mmol, 66.32% yield). LCMS (ESI+): 353/355 (M+H, Br pattern), ¹H-NMR (400 MHz, CDCl₃): 7.31 (d, J=8.4 Hz, 2H), 6.34 (d, J=8.4 Hz, 2H), 4.11 (s, 4H), 3.96 (s, 4H), 1.47 (s, 9H).

### Step 2: tert-butyl 6-[4-(4,4,5,5-tetramethyl-1,3,2-dioxaborolan-2-yl)phenyl]-2,6-diazaspiro[3.3]heptane-2-carboxylate

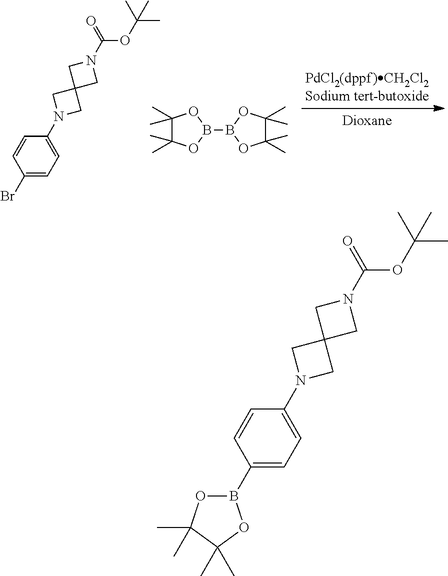

To a solution of tert-butyl 6-(4-bromophenyl)-2,6-diazaspiro[3.3]heptane-2-carboxylate (6.2 g, 17.55 mmol), 4,4,5,5-tetramethyl-2-(4,4,5,5-tetramethyl-1,3,2-dioxaborolan-2-yl)-1,3,2-dioxaborolane (5.79 g, 22.82 mmol), and Potassium Acetate (5.17 g, 52.65 mmol, 3.29 mL) in 1,4-dioxane (48 mL) was added 1,1′-Bis(Diphenylphosphino)ferrocenepalladium (II) dichloride dichloromethane (716.65 mg, 877.56 μmol) and the mixture was stirred at 80° C. for 16 hours. The mixture was cooled and filtered through a pad of celite, then was concentrated under reduced pressure. The crude residue was purified by silica gel chromatography (0% to 50% Ethyl acetate in hexanes) to give tert-butyl 6-[4-(4,4,5,5-tetramethyl-1,3,2-dioxaborolan-2-yl)phenyl]-2,6-diazaspiro[3.3]heptane-2-carboxylate (6.32 g, 15.79 mmol, 89.95% yield). LCMS (ESI+): MS (ESI+): 400.3/401.3/402.3 (M+H, Boron pattern); 1H NMR (400 MHz, DMSO-d₆) δ 7.48 (d, J=8.3 Hz, 2H), 6.38 (d, J=8.3 Hz, 2H), 4.03 (s, 4H), 3.96 (s, 4H), 1.39 (s, 9H), 1.26 (s, 12H).

### Step 3: tert-butyl 6-[4-[2-[1-(6,7-dihydro-5H-pyrrolo[1,2-c]imidazol-1-yl)-2-ethoxy-2-oxo-ethyl]-7-fluoro-3-oxo-isoindolin-5-yl]phenyl]-2,6-diazaspiro[3.3]heptane-2-carboxylate

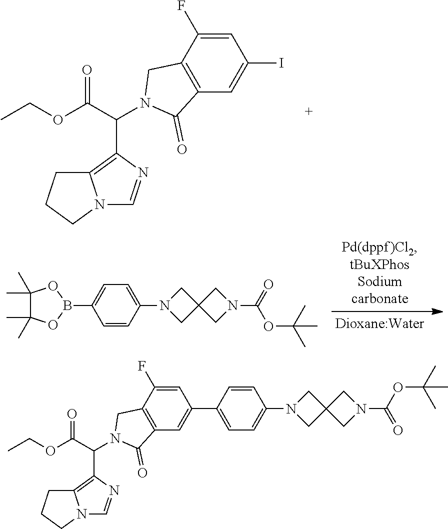

Ethyl 2-(6,7-dihydro-5H-pyrrolo[1,2-c]imidazol-1-yl)-2-(4-fluoro-6-iodo-1-oxo-isoindolin-2-yl)acetate (1.57 g, 3.35 mmol) and tert-butyl 6-[4-(4,4,5,5-tetramethyl-1,3,2-dioxaborolan-2-yl)phenyl]-2,6-diazaspiro[3.3]heptane-2-carboxylate (1.74 g, 4.35 mmol) were dissolved in dioxane (10 mL) and tBuXPhos (422.40 mg, 669.16 μmol) was added, followed by Sodium carbonate (780.16 mg, 7.36 mmol) dissolved in Water (2.5 mL). The mixture was degassed with argon and 1,1′-Bis(Diphenylphosphino)ferrocenepalladium (II) dichloride (293.69 mg, 401.49 μmol) was added. The reaction was sealed and heated at 80° C. on a heating block for 4 hours. The mixture was concentrated and purified by flash column chromatography on silica gel (0-100% ethyl acetate in hexane) to give tert-butyl 6-[4-[2-[1-(6,7-dihydro-5H-pyrrolo[1,2-c]imidazol-1-yl)-2-ethoxy-2-oxo-ethyl]-7-fluoro-3-oxo-isoindolin-5-yl]phenyl]-2,6-diazaspiro[3.3]heptane-2-carboxylate (1.8 g, 2.92 mmol, 87.38% yield) as a yellow oil. LCMS (ESI+): 616.2 (M+H)

### Step 4: [2-[6-[4-(2-tert-butoxycarbonyl-2,6-diazaspiro[3.3]heptan-6-yl)phenyl]-4-fluoro-1-oxo-isoindolin-2-yl]-2-(6,7-dihydro-5H-pyrrolo[1,2-c]imidazol-1-yl)acetyl]oxylithium

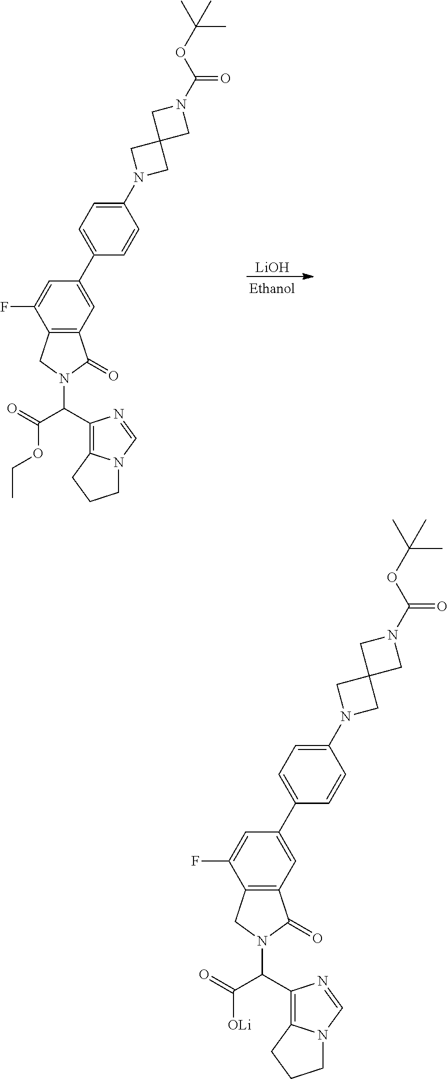

### Step 5: tert-butyl 6-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]-2,6-diazaspiro[3.3]heptane-2-carboxylate

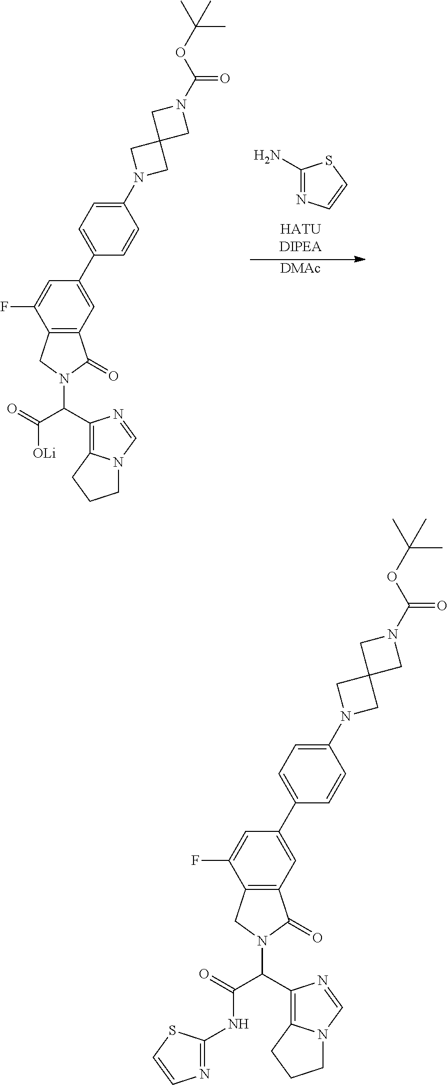

Thiazol-2-amine (106.29 mg, 1.06 mmol) and [2-[6-[4-(2-tert-butoxycarbonyl-2,6-diazaspiro[3.3]heptan-6-yl)phenyl]-4-fluoro-1-oxo-isoindolin-2-yl]-2-(6,7-dihydro-5H-pyrrolo[1,2-c]imidazol-1-yl)acetyl]oxylithium (600 mg, 1.01 mmol) were mixed in DMAc (5 mL) and cooled to 0° C. N,N-Diisopropylethylamine (522.56 mg, 4.04 mmol, 704.26 μL) was added to the reaction mixture, and HATU (499.65 mg, 1.31 mmol) was added, and the reaction mixture was stirred for 30 min at 0° C. The reaction mixture was warmed to 20° C. and stirred for 2 hours. The reaction mixture was diluted with saturated aqueous NaHCO₃ and extracted with ethyl acetate. The organic layers were washed with water and brine, dried over Na₂SO₄, filtered and concentrated in vacuo. The crude material was purified by silica gel chromatography (0-20% methanol in dichloromethane) to afford tert-butyl 6-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]-2,6-diazaspiro[3.3]heptane-2-carboxylate (630 mg, 940.63 μmol, 93.06% yield). LCMS (ESI+): 670.3 (M+H).

### Step 6: 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide, trifluoroacetic acid salt

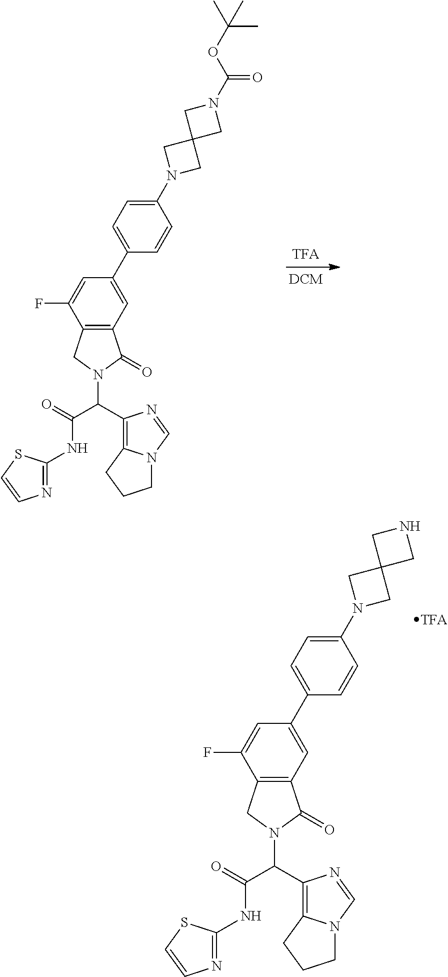 [SMILES: CC(C)(C)OC(=O)N1CC2(C1)CN(c1ccc(-c3cc(F)c4c(c3)C(=O)N(C(C(=O)Nc3nccs3)c3ncn5c3CCC5)C4)cc1)C2 ; O=C(Nc1nccs1)C(c1ncn2c1CCC2)N1Cc2c(F)cc(-c3ccc(N4CC5(CNC5)C4)cc3)cc2C1=O]

tert-Butyl 6-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]-2,6-diazaspiro[3.3]heptane-2-carboxylate (86 mg, 128.40 μmol) was dissolved in dichloromethane (2 mL) and Trifluoroacetic acid (585.64 mg, 5.14 mmol, 395.70 μL) was added. The reaction mixture was stirred for 2 h. The reaction mixture was added dropwise under stirring to MTBE (10 mL). The precipitate was allowed to settle, and the supernatant was decanted and discarded. The resulting solid was submitted to high vacuum to afford 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide, trifluoroacetic acid salt (107 mg, 134.14 μmol, quantitative yield). LCMS (ESI+): 564.2 (M+H)

### Step 6B Synthesis of 2-[6-[4-(2-acetyl-2,6-diazaspiro[3.3]heptan-6-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide

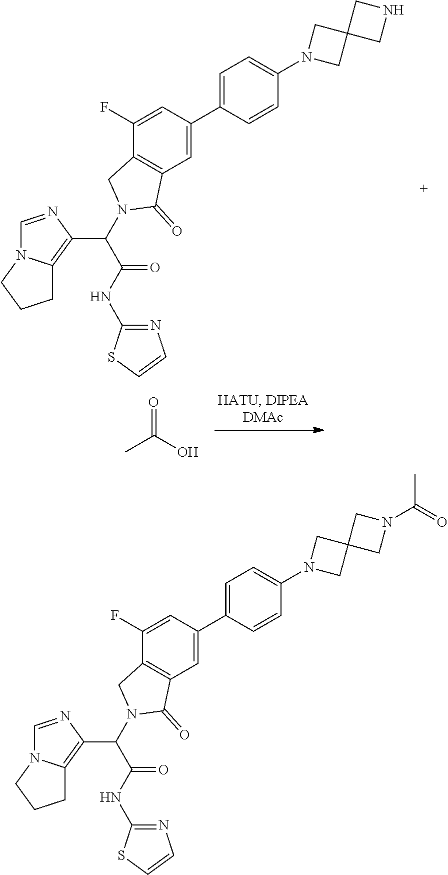

Compound 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide (27 mg, 39.49 μmol, TFA salt) and acetic acid (5.93 mg, 98.73 μmol, 5.65 μL) were mixed in DMF, and the reaction mixture was cooled to 0° C. Then DIPEA (25.52 mg, 197.46 μmol, 34.39 μL) and HATU (18.02 mg, 47.39 μmop were added to the mixture, and the reaction was stirred for 2 hours. Upon completion of the reaction, the mixture was purified by reverse-phase column chromatography (50 g C18 column, 0% to 100% acetonitrile in water+0.1% TFA as eluent). The pure fractions were pooled and partitioned between ethyl acetate and aqueous saturated sodium bicarbonate solution. The organic layer was washed with brine, dried over sodium sulfate, filtered and evaporated under reduced pressure. The residue was then purified by column chromatography (24 g silica gel, 0-20% MeOH in EtOAc) to afford 2-[6-[4-(2-acetyl-2,6-diazaspiro[3.3]heptan-6-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide (10.2 mg, 16.59 μmol, 42.01% yield). ¹H NMR (400 MHz, DMSO-d₆): δ 12.51 (s, 1H), 7.73 (d, J=1.4 Hz, 1H), 7.69 (dd, J=10.6, 1.4 Hz, 1H), 7.66-7.62 (m, 2H), 7.60 (s, 1H), 7.48 (d, J=3.6 Hz, 1H), 7.25 (d, J=3.6 Hz, 1H), 6.68-6.38 (m, 2H), 6.14 (s, 1H), 4.79 (d, J=17.7 Hz, 1H), 4.30 (s, 2H), 4.21 (d, J=17.7 Hz, 1H), 4.14-3.85 (m, 8H), 2.88-2.69 (m, 1H), 2.58-2.37 (m, 1H), 1.75 (s, 3H). LCMS (ES⁺): m/z 612.2 [M+H]⁺.

### Step 7: 2-(6,7-dihydro-5H-pyrrolo[1,2-c]imidazol-1-yl)-2-[6-[4-[2-[2-[4-[4-[(2,6-dioxo-3-piperidyl)amino]phenyl]-1-piperidyl]acetyl]-2,6-diazaspiro[3.3]heptan-6-yl]phenyl]-4-fluoro-1-oxo-isoindolin-2-yl]-N-thiazol-2-yl-acetamide

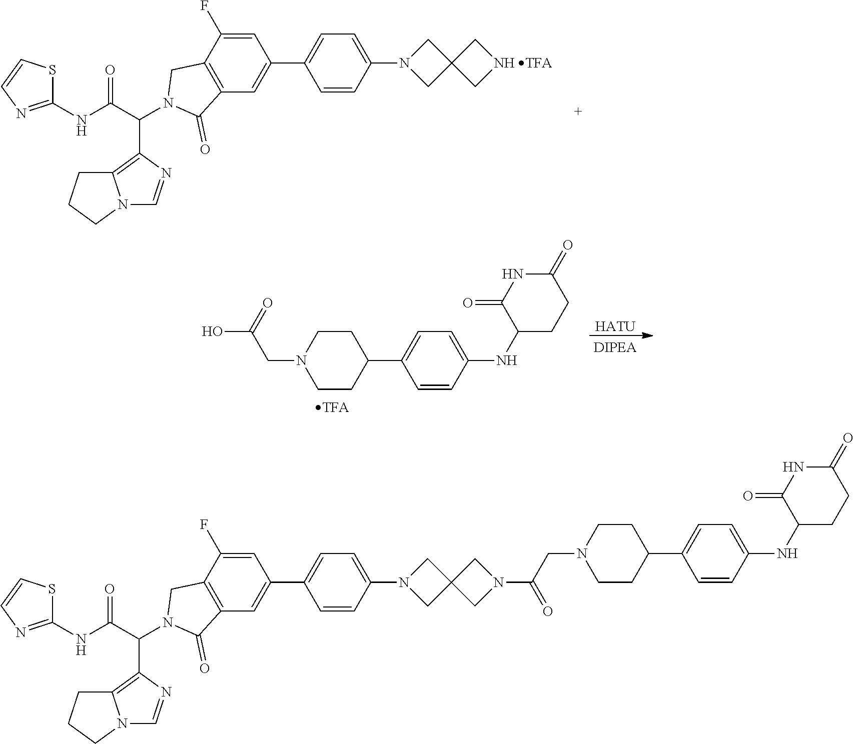

2-[6-[4-(2,6-Diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide, trifluoroacetic acid salt (113 mg, 165.28 μmol) and 2-[4-[4-[(2,6-dioxo-3-piperidyl)amino]phenyl]-1-piperidyl]acetic acid, trifluoroacetic acid (91.12 mg, 198.34 μmol) were mixed in DMF and cooled to 0° C. N,N-Diisopropylethylamine (106.81 mg, 826.42 μmol, 143.94 μL) was added to the reaction mixture, and HATU (81.70 mg, 214.87 μmol) was added, and the reaction mixture was stirred for 1 h at 0° C. The reaction mixture was acidified with 4-5 drops of TFA, and injected directly on a RP C18 column (50 g C18) for purification (5% to 100% acetonitrile (+0.1% TFA) in water (+0.1% TFA) over 12 minutes). The desired fractions were neutralized with aqueous NaHCO₃ (ca. 60 mL) and extracted with 1:4 isopropanol:chloroform mixture. The organic layer was dried over Na₂SO₄, filtered, and evaporated under reduced pressure to afford a solid. The solid was dissolved in dichloromethane, and purified by silica gel chromatography (0% to 20% methanol in dichloromethane). The desired fractions were evaporated under reduced pressure, then dissolved in dichloromethane, transferred to a 8 mL vial, and evaporated under reduced pressure. 4 mL water+4 mL acetonitrile were added, and the mixture was thoroughly sonicated, vortexed and sonicated again. The suspension was frozen and lyophilized to afford Compound 47 (56 mg, 59.31 μmol, 35.88% yield). LCMS (ESI+): 897.4 (M+H), LCMS (ESI−): 895.3 (M−H), ¹H NMR (400 MHz, DMSO-d₆) δ 12.44 (s, 1H), 10.69 (s, 1H), 7.67 (s, 1H), 7.63 (d, J=10.7 Hz, 2H), 7.57 (d, J=8.5 Hz, 2H), 7.54 (s, 1H), 7.42 (d, J=3.5 Hz, 1H), 7.19 (d, J=3.6 Hz, 1H), 6.90 (d, J=8.2 Hz, 2H), 6.54 (d, J=8.3 Hz, 2H), 6.47 (d, J=8.3 Hz, 2H), 6.08 (s, 1H), 5.57 (d, J=7.4 Hz, 1H), 4.73 (d, J=17.7 Hz, 1H), 4.36 (s, 2H), 4.30-4.07 (m, 2H), 4.01 (s, 2H), 3.96 (s, 6H), 3.91 (m, 1H), 2.92 (s, 2H), 2.82 (m, 2H), 2.76-2.58 (m, 2H), 2.56-2.46 (m, 1H), 2.01 (m, 3H), 1.80 (m, 1H), 1.55 (m, 4H).

### Example 25. Synthesis of 2-(6,7-dihydro-5H-pyrrolo[1,2-c]imidazol-1-yl)-2-[6-[4-[2-[2-[4-[4-[[(3S)-2,6-dioxo-3-piperidyl]amino]-2-fluoro-phenyl]-1-piperidyl]acetyl]-2,6-diazaspiro[3.3]heptan-6-yl]phenyl]-4-fluoro-1-oxo-isoindolin-2-yl]-N-thiazol-2-yl-acetamide (Compound 48)

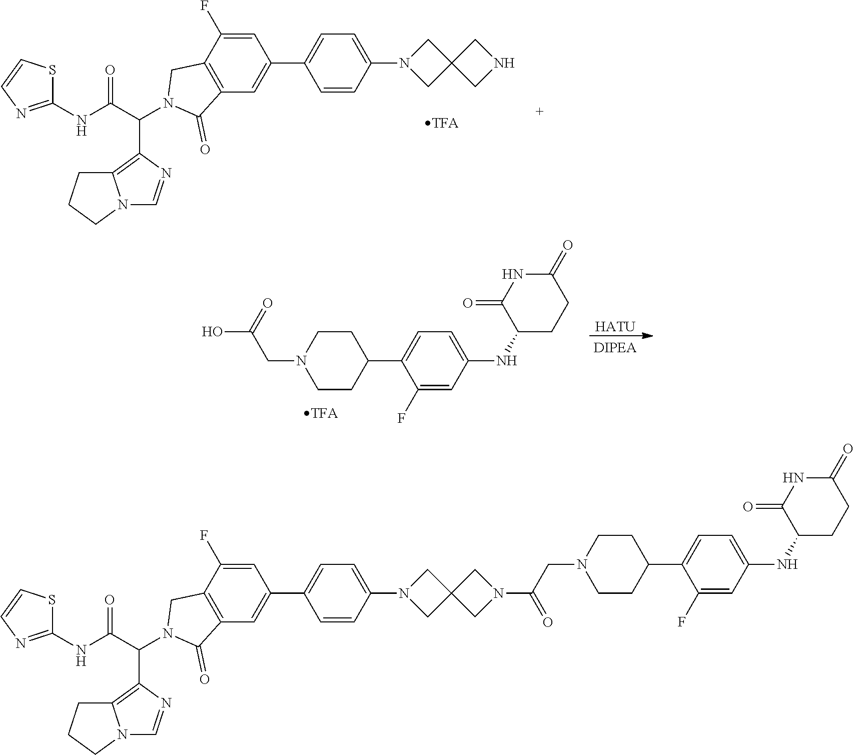

2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide trifluoroacetic acid (40 mg, 58.51 μmol) and 2-[4-[4-[[(3S)-2,6-dioxo-3-piperidyl]amino]-2-fluoro-phenyl]-1-piperidyl]acetic acid trifluoroacetic acid (33.52 mg, 70.21 μmol) were mixed in DMF and cooled to 0° C. N,N-Diisopropylethylamine (37.81 mg, 292.54 μmol, 50.95 μL) was added to the reaction mixture, and HATU (28.92 mg, 76.06 μmol) was added, and the reaction mixture was stirred for 1 h at 0° C. The reaction mixture was acidified with 4-5 drops of TFA, and injected directly on a RP C18 column (50 g C18) for purification (5% to 100% can (+0.1% TFA) in water (+0.1% TFA) over 12 minutes). The desired fractions were neutralized with aqueous NaHCO₃ (ca. 60 mL), extracted twice with a 1:4 isopropanol: chloroform mixture. The organic layer was dried over Na₂SO₄, filtered, and evaporated under d reduced pressure to afford a solid. The solid was dissolved in dichloromethane, and purified by silica gel chromatography (0% to 20% methanol in dichloromethane). The desired fractions were evaporated under reduced pressure, then dissolved in dichloromethane, transferred to a 8 mL vial, and evaporated under reduced pressure. Water (1 mL) and acetonitrile (1 mL) were added, and the mixture was thoroughly sonicated, vortexed and sonicated again. The suspension was frozen and lyophilized to afford Compound 48 (27.9 mg, 30.19 μmol, 51.59% yield). LCMS (ESI+): 915.3 (M+H), ¹H NMR (400 MHz, DMSO-d₆) δ 12.51 (s, 1H), 10.77 (s, 1H), 7.74 (d, J=1.3 Hz, 1H), 7.70 (dd, J=10.7, 1.4 Hz, 1H), 7.64 (d, J=8.5 Hz, 2H), 7.60 (s, 1H), 7.48 (d, J=3.5 Hz, 1H), 7.25 (d, J=3.6 Hz, 1H), 7.01 (t, J=8.8 Hz, 1H), 6.54 (d, J=8.4 Hz, 2H), 6.50-6.34 (m, 2H), 6.15 (s, 1H), 5.99 (d, J=7.7 Hz, 1H), 4.79 (d, J=17.7 Hz, 1H), 4.43 (s, 2H), 4.30 (ddd, J=12.1, 7.7, 4.8 Hz, 1H), 4.21 (d, J=17.6 Hz, 1H), 4.08 (s, 2H), 4.02 (s, 3H), 4.01-3.87 (m, 2H), 3.16-2.83 (m, 4H), 2.83-2.64 (m, 2H), 2.64-2.52 (m, 1H), 2.49-2.38 (m, 1H), 2.22-1.97 (m, 3H), 1.86 (qd, J=12.0, 4.5 Hz, 1H), 1.65 (s, 4H).

### Example 26. Synthesis of 2-(6,7-dihydro-5H-pyrrolo[1,2-c]imidazol-1-yl)-2-[6-[4-[2-[2-[4-[3-(2,4-dioxohexahydropyrimidin-1-yl)-1-methyl-indazol-6-yl]-1-piperidyl]acetyl]-2,6-diazaspiro[3.3]heptan-6-yl]phenyl]-4-fluoro-1-oxo-isoindolin-2-yl]-N-thiazol-2-yl-acetamide (Compound 49)

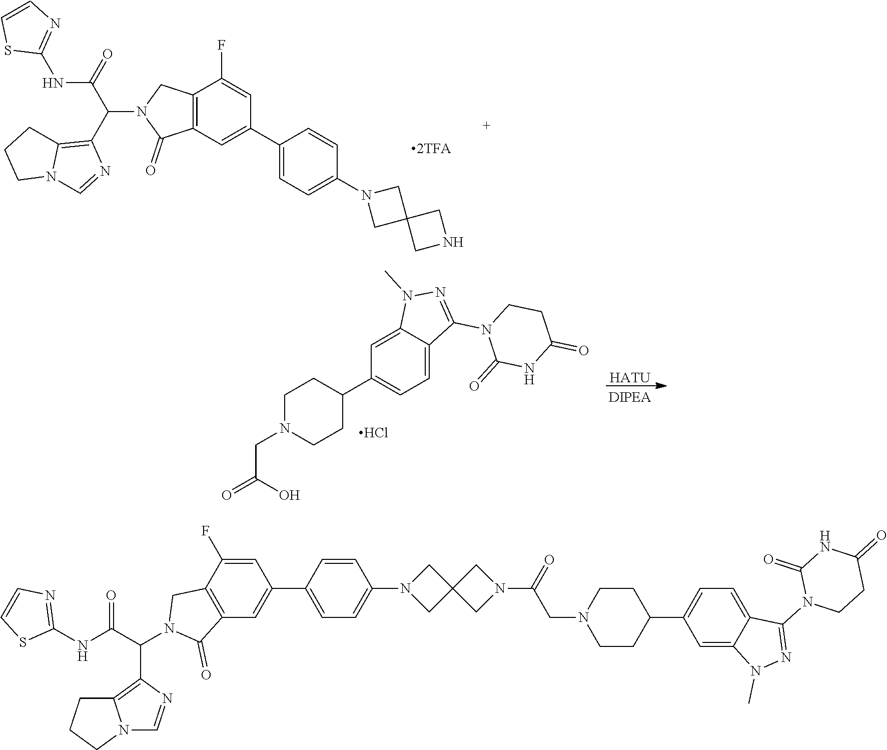

2-[4-[3-(2,4-Dioxohexahydropyrimidin-1-yl)-1-methyl-indazol-6-yl]-1-piperidyl]acetic acid hydrochloride (52.89 mg, 125.36 μmol) was dissolved in DMF (0.5 mL) and cooled to 0° C. N,N-diisopropylethylamine (40.50 mg, 313.40 μmol, 54.59 μL) was added to the reaction mixture. HATU (35.75 mg, 94.02 μmol) was added and the reaction mixture was stirred at 35° C. for 10 minutes. The solution was cooled to 0° C. and 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide bis trifluoroacetic acid salt (50 mg, 62.68 μmol, 062) was added. The reaction mixture was warmed to room temperature and stirred for 2 hours. The mixture was injected on a 50 g C18 column, and purified using a 0% to 100% Acetonitrile in water+0.1% TFA water elution gradient. Desired fractions were pooled and partitioned between ethyl acetate and sodium bicarbonate (aqueous, aqueous). The organic layer was washed with brine, dried with sodium sulfate, filtered and evaporated under reduced pressure. The crude residue was purified by silica gel chromatography (0% to 20% methanol in dichloromethane). Desired fractions were evaporated to afford Compound 49 (35 mg, 37.35 μmol, 59.59% yield). LCMS (ESI+): 937.2 (M+H), LCMS (ESI−): 935.2 (M−H), ¹H NMR (400 MHz, DMSO-d₆) δ 12.45 (s, 1H), 10.46 (s, 1H), 7.67 (s, 1H), 7.63 (d, J=11.0 Hz, 1H), 7.57 (d, J=8.6 Hz, 2H), 7.54 (s, 1H), 7.49 (d, J=8.5 Hz, 1H), 7.42 (d, J=3.6 Hz, 1H), 7.38 (s, 1H), 7.19 (d, J=3.5 Hz, 1H), 6.99 (d, J=8.5 Hz, 1H), 6.48 (d, J=8.5 Hz, 2H), 6.08 (s, 1H), 4.72 (d, J=17.7 Hz, 1H), 4.39 (s, 2H), 4.15 (d, J=17.6 Hz, 1H), 4.02 (s, 2H), 3.97 (s, 3H), 3.97-3.91 (m, 1H), 3.90 (s, 3H), 3.84 (t, J=6.7 Hz, 2H), 3.03-2.79 (m, 4H), 2.68 (t, J=6.6 Hz, 3H), 2.60-2.45 (m, 1H), 2.38-2.25 (m, 1H), 2.19-2.02 (m, 2H), 1.99-1.85 (m, 1H), 1.73 (s, 5H).

### Example 27. Synthesis of 2-(6,7-dihydro-5H-pyrrolo[1,2-c]imidazol-1-yl)-2-(6-(4-(6-(2-(4-(4-((2,6-dioxopiperidin-3-yl)amino)phenyl)-3,3-difluoropiperidin-1-yl)acetyl)-2,6-diazaspiro[3.3]heptan-2-yl)phenyl)-4-fluoro-1-oxoisoindolin-2-yl)-N-(thiazol-2-yl)acetamide, Isomer 2, (Compound 52)

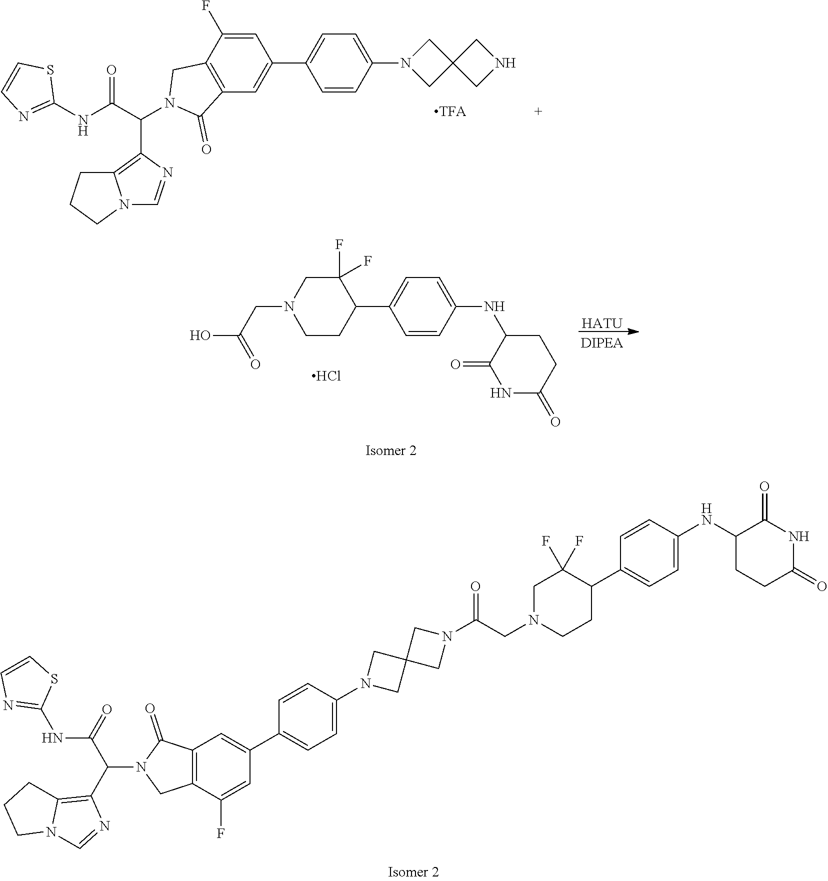

Compound 52 was synthesized in 19.5% using the same procedure as 2-(6,7-dihydro-5H-pyrrolo[1,2-c]imidazol-1-yl)-2-(6-(4-(6-(2-(4-(4-(2,6-dioxopiperidin-3-yl)amino)phenyl)-3,3-difluoropiperidin-1-yl)acetyl)-2,6-diazaspiro[3.3]heptan-2-yl)phenyl)-4-fluoro-1-oxoisoindolin-2-yl)-N-(thiazol-2-yl)acetamide, using 2-[4-[4-[(2,6-dioxo-3-piperidyl)amino]phenyl]-3,3-difluoro-1-piperidyl]acetic acid hydrochloride, isomer 2 instead of 2-[4-[4-[(2,6-dioxo-3-piperidyl)amino]phenyl]-3,3-difluoro-1-piperidyl]acetic acid hydrochloride isomer 1. LCMS (ESI+): 933.4 (M+H), ¹H NMR (400 MHz, DMSO-d₆) δ 12.52 (s, 1H), 10.78 (s, 1H), 7.75 (d, J=1.3 Hz, 1H), 7.71 (dd, J=10.7, 1.4 Hz, 1H), 7.65 (d, J=8.7 Hz, 2H), 7.61 (s, 1H), 7.49 (d, J=3.5 Hz, 1H), 7.26 (d, J=3.6 Hz, 1H), 7.02 (d, J=8.2 Hz, 2H), 6.64 (d, J=8.4 Hz, 2H), 6.55 (d, J=8.7 Hz, 2H), 6.15 (s, 1H), 5.80 (d, J=7.5 Hz, 1H), 4.80 (d, J=17.7 Hz, 1H), 4.42 (s, 2H), 4.30 (ddd, J=12.0, 7.6, 4.9 Hz, 1H), 4.22 (d, J=17.7 Hz, 1H), 4.10 (s, 2H), 4.07-3.93 (m, 6H), 3.23-3.07 (m, 3H), 2.97-2.87 (m, 2H), 2.88-2.67 (m, 3H), 2.67-2.44 (m, 4H), 2.43-2.31 (m, 1H), 2.16-1.97 (m, 2H), 1.88 (qd, J=12.2, 4.8 Hz, 1H), 1.71 (d, J=13.3 Hz, 1H).

### Example 28 Synthesis of 2-(6,7-dihydro-5H-pyrrolo[1,2-c]imidazol-1-yl)-2-[6-[4-[2-[2-[4-[4-[[(3S)-2,6-dioxo-3-piperidyl]amino]-2-fluoro-phenyl]-1-piperidyl]acetyl]-2,6-diazaspiro[3.3]heptan-6-yl]phenyl]-4-fluoro-1-oxo-isoindolin-2-yl]-N-(2-pyridyl)acetamide (Compound 53)

### Step 1: tert-butyl 6-[4-[2-[1-(6,7-dihydro-5H-pyrrolo[1,2-c]imidazol-1-yl)-2-oxo-2-(2-pyridylamino)ethyl]-7-fluoro-3-oxo-isoindolin-5-yl]phenyl]-2,6-diazaspiro[3.3]heptane-2-carboxylate

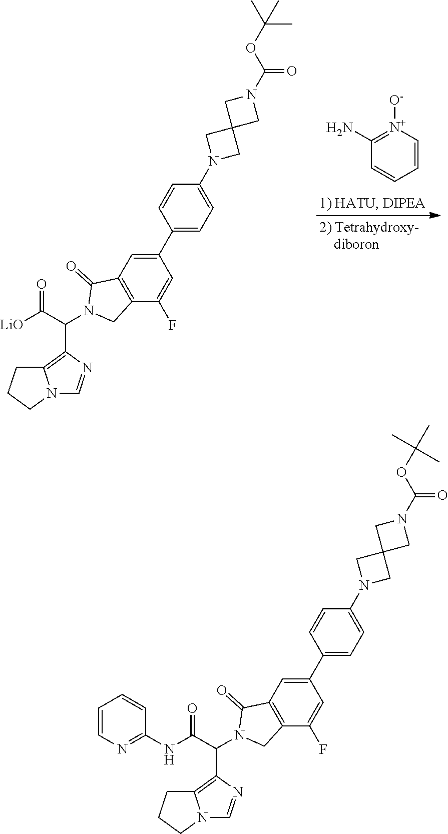

To a solution of 1-oxidopyridin-1-ium-2-amine (43.67 mg, 396.58 μmol) and [2-[6-[4-(2-tert-butoxycarbonyl-2,6-diazaspiro[3.3]heptan-6-yl)phenyl]-4-fluoro-1-oxo-isoindolin-2-yl]-2-(6,7-dihydro-5H-pyrrolo[1,2-c]imidazol-1-yl)acetyl]oxylithium (214 mg, 360.53 μmol) in DMF (3 mL) was added N,N-Diisopropylethylamine (186.38 mg, 1.44 mmol, 251.18 μL) and HATU (178.21 mg, 468.69 μmol) at ambient temperature. After 15 minutes, tetrahydroxydiboron (96.96 mg, 1.08 mmol) was added and stirred for 30 minutes. The reaction mixture was diluted with a water:brine mixture (1:1), extracted with Ethyl Acetate, washed with brine, dried over Na₂SO₄, and concentrated. The crude residue was purified by silica gel column chromatography (24 grams, 0% to 20% Methanol in dichloromethane) to afford tert-butyl 6-[4-[2-[1-(6,7-dihydro-5H-pyrrolo[1,2-c]imidazol-1-yl)-2-oxo-2-(2-pyridylamino)ethyl]-7-fluoro-3-oxo-isoindolin-5-yl]phenyl]-2,6-diazaspiro[3.3]heptane-2-carboxylate (175 mg, 263.66 μmol, 73.13% yield) as a yellow solid. LCMS (ESI+): 664.3 (M+H)/608.3 (M-tBu+H)

### Step 2: 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-(2-pyridyl)acetamide, bis-trifluoroacetic acid salt

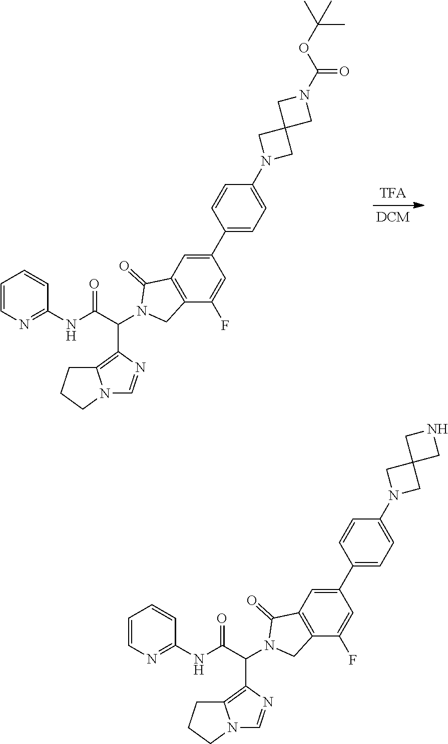 [SMILES: CC(C)(C)OC(=O)N1CC2(C1)CN(c1ccc(-c3cc(F)c4c(c3)C(=O)N(C(C(=O)Nc3ccccn3)c3ncn5c3CCC5)C4)cc1)C2 ; O=C(Nc1ccccn1)C(c1ncn2c1CCC2)N1Cc2c(F)cc(-c3ccc(N4CC5(CNC5)C4)cc3)cc2C1=O]

tert-Butyl 6-[4-[2-[1-(6,7-dihydro-5H-pyrrolo[1,2-c]imidazol-1-yl)-2-oxo-2-(2-pyridylamino)ethyl]-7-fluoro-3-oxo-isoindolin-5-yl]phenyl]-2,6-diazaspiro[3.3]heptane-2-carboxylate (120 mg, 180.79 μmol) was dissolved in dichloromethane (2 mL). Trifluoroacetic acid (278 μL, 3.61 mmol) was added, and the reaction mixture was stirred at 35° C. for 4 hours. The reaction mixture was evaporated under pressure, frozen, and submitted to high vacuum to afford 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-(2-pyridyl)acetamide, bis-trifluoroacetic acid salt (144 mg, 181.89 μmol, quantitative yield). LCMS (ESI+): 564.3 (M+H)

### Step 3: 2-(6,7-dihydro-5H-pyrrolo[1,2-c]imidazol-1-yl)-2-[6-[4-[2-[2-[4-[4-[[(3S)-2,6-dioxo-3-piperidyl]amino]-2-fluoro-phenyl]-1-piperidyl]acetyl]-2,6-diazaspiro[3.3]heptan-6-yl]phenyl]-4-fluoro-1-oxo-isoindolin-2-yl]-N-(2-pyridyl)acetamide

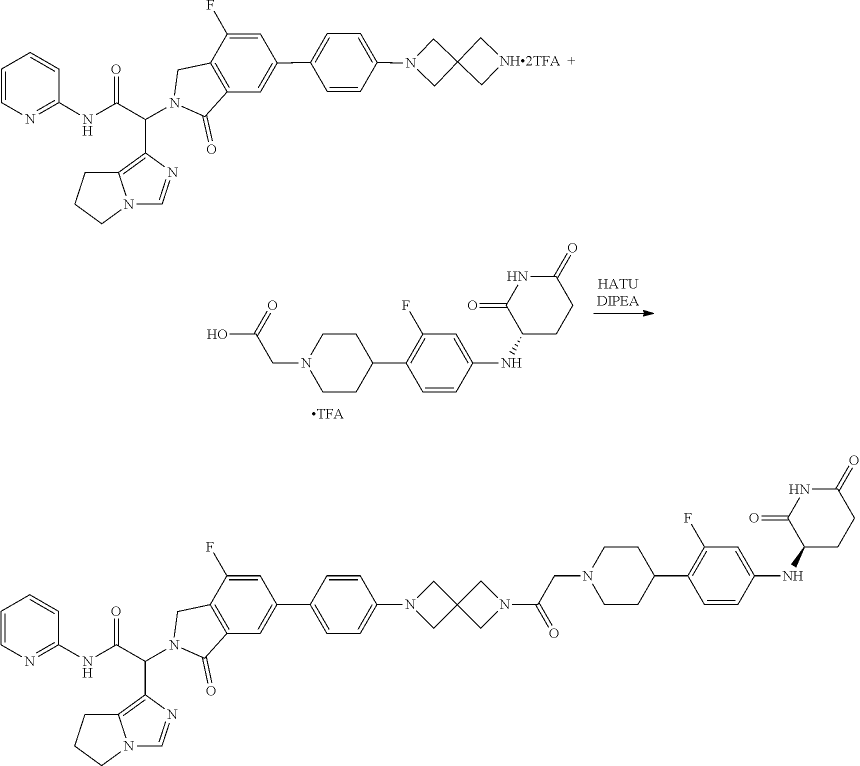

2-[6-[4-(2,6-Diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-(2-pyridyl)acetamide bis-trifluoroacetic acid salt (111 mg, 163.80 μmol) and 2-[4-[4-[[(3S)-2,6-dioxo-3-piperidyl]amino]-2-fluoro-phenyl]-1-piperidyl]acetic acid trifluoroacetic acid salt (93.84 mg, 196.56 μmol) were mixed in DMF and cooled to 0° C. N,N-Diisopropylethylamine (105.85 mg, 819.01 μmol, 142.65 μL) was added to the reaction mixture, and HATU (80.97 mg, 212.94 μmol) was added, and the reaction mixture was stirred for 1 h at 0° C. The reaction mixture was acidified with 4-5 drops of TFA, and injected directly on a RP C18 column (50 g C18) for purification using a 5% to 100% acetonitrile (+0.1% TFA) in water (+0.1% TFA) eluent gradient. The desired fractions were neutralized with aqueous aqueous NaHCO₃ (ca. 60 mL) and extracted with 1:4 isopropanol:chloroform. The organic layer was dried over Na₂SO₄, filtered, and evaporated under reduced pressure to afford a solid. The solid was dissolved in dichloromethane, and purified by silica gel chromatography (0% to 20% methanol in dichloromethane). The desired fractions were evaporated under reduced pressure. The crude residue was dissolved in dichloromethane, transferred to a 8 mL vial, and evaporated under reduced pressure. Water (1 mL) and acetonitrile (1 mL) were added, and the mixture was thoroughly sonicated, vortexed and sonicated again. The suspension was frozen and lyophilized to afford Compound 53 (4 mg, 4.18 μmol, 2.55% yield, 95% purity). LCMS (ESI+): 909.4 (M+H), ¹H NMR (400 MHz, DMSO-d₆) δ 10.81 (s, 1H), 10.71 (s, 2H), 8.37-8.09 (m, 1H), 8.00 (d, J=8.4 Hz, 1H), 7.89-7.31 (m, 7H), 7.05 (dd, J=7.3, 4.8 Hz, 1H), 6.92 (q, J=11.4, 10.2 Hz, 1H), 6.48 (d, J=8.3 Hz, 2H), 6.43-6.21 (m, 2H), 6.12 (s, 1H), 5.94 (d, J=7.6 Hz, 1H), 4.72 (d, J=17.7 Hz, 1H), 4.35 (s, 2H), 4.23 (dt, J=12.2, 6.8 Hz, 1H), 4.14 (d, J=17.7 Hz, 1H), 4.03 (s, 2H), 3.96 (s, 4H), 3.95-3.77 (m, 1H), 3.11-2.46 (m, 4H), 2.20-1.87 (m, 2H), 1.88-1.24 (m, 7H).

### Example 29. Synthesis of 2-(6,7-dihydro-5H-pyrrolo[1,2-c]imidazol-1-yl)-2-[6-[4-[2-[2-[4-[3-(2,4-dioxohexahydropyrimidin-1-yl)-1-methyl-indazol-6-yl]-1-piperidyl]acetyl]-2,6-diazaspiro[3.3]heptan-6-yl]phenyl]-4-fluoro-1-oxo-isoindolin-2-yl]-N-(2-pyridyl)acetamide (Compound 54)

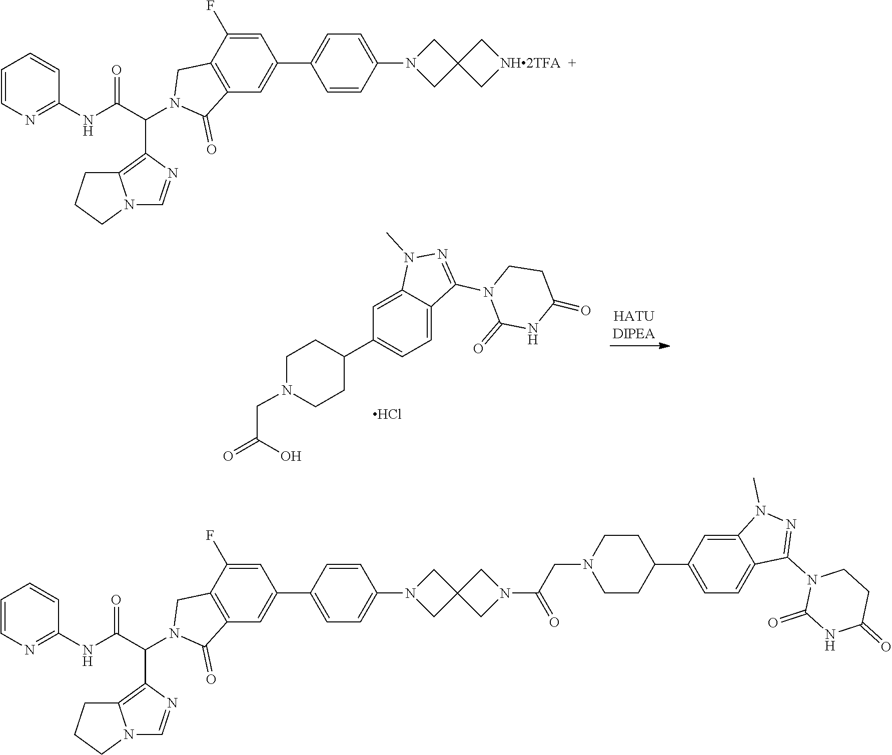

2-[4-[3-(2,4-Dioxohexahydropyrimidin-1-yl)-1-methyl-indazol-6-yl]-1-piperidyl]acetic acid hydrochloride (28.61 mg, 67.87 μmol) were mixed in DMF, the reaction mixture was cooled to 0° C. N,N-diisopropylethylamine (22.04 mg, 170.53 μmol, 29.70 μL) was added to the reaction mixture, and HATU (19.45 mg, 51.16 μmol) was added, and the reaction mixture was stirred at 35° C. for 10 minutes. The reaction mixture was cooled to 0° C. 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-(2-pyridyl)acetamide, bis trifluoroacetic acid salt (27 mg, 34.11 μmol) was added in one portion, and the reaction mixture was stirred for 2 hours while warming to 20° C. The mixture was injected on a 50 g C18 column and purified using a 0% to 100% Acetonitrile in water+0.1% TFA water elution gradient. Desired fractions were neutralized with sodium bicarbonate (aqueous, aqueous), and the aqueous mixture was extracted twice with an isopropanol:chloroform mixture (1:4). The organic layer was washed with brine, dried with sodium sulfate, filtered and evaporated under reduced pressure. The residue was purified by silica gel chromatography (0% to 20% methanol in dichloromethane) to afford Compound 54 (16 mg, 16.33 μmol, 47.87% yield). LCMS (ESI+): 931.3 (M+H), LCMS (ESI−): 929.3 (M−H), ¹H NMR (400 MHz, DMSO-d₆) δ 10.88 (s, 1H), 10.53 (s, 1H), 8.32 (ddd, J=4.9, 1.9, 0.9 Hz, 1H), 8.07 (d, J=8.4 Hz, 1H), 7.87-7.75 (m, 1H), 7.74 (d, J=1.4 Hz, 1H), 7.69 (dd, J=10.6, 1.4 Hz, 1H), 7.67-7.61 (m, 2H), 7.60 (s, 1H), 7.56 (d, J=8.4 Hz, 1H), 7.45 (s, 1H), 7.12 (ddd, J=7.4, 4.9, 1.0 Hz, 1H), 7.05 (dd, J=8.6, 1.3 Hz, 1H), 6.68-6.36 (m, 2H), 6.19 (s, 1H), 4.79 (d, J=17.7 Hz, 1H), 4.45 (s, 2H), 4.21 (d, J=17.7 Hz, 1H), 4.09 (s, 2H), 4.04 (s, 3H), 4.01 (s, 1H), 3.97 (s, 3H), 3.90 (t, J=6.7 Hz, 2H), 3.18-2.87 (m, 4H), 2.87-2.70 (m, 3H), 2.70-2.57 (m, 1H), 2.59-2.50 (m, 5H), 2.31-2.08 (m, 2H), 1.92-1.60 (m, 4H).

### Example 30. Synthesis of 2-(6,7-dihydro-5H-pyrrolo[1,2-c]imidazol-1-yl)-2-[6-[4-[[4-[2-[4-[4-[[(3S)-2,6-dioxo-3-piperidyl]amino]-2-fluoro-phenyl]-1-piperidyl]acetyl]piperazin-1-yl]methyl]phenyl]-4-fluoro-1-oxo-isoindolin-2-yl]-N-thiazol-2-yl-acetamide, (Compound 58)

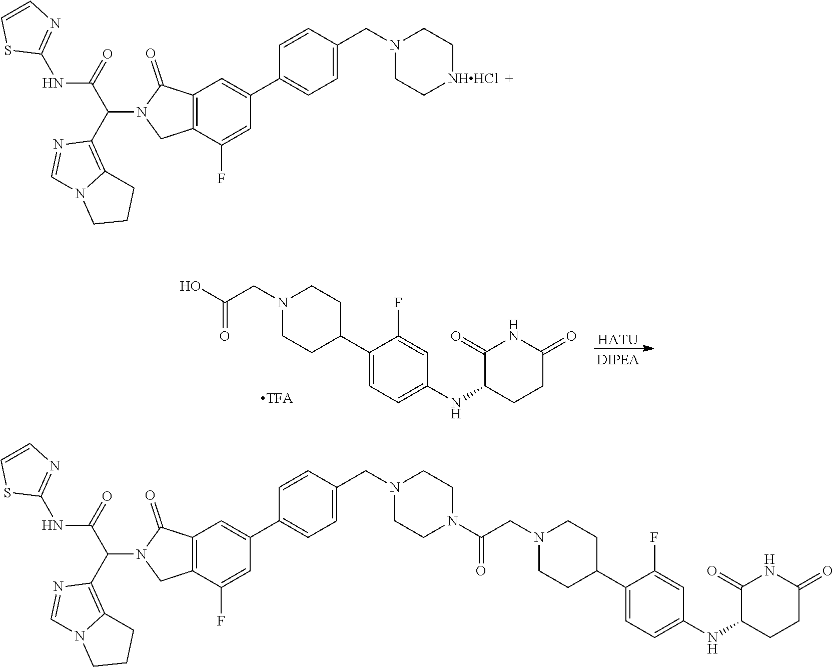

2-(6,7-Dihydro-5H-pyrrolo[1,2-c]imidazol-1-yl)-2-[4-fluoro-1-oxo-6-[4-(piperazin-1-ylmethyl)phenyl]isoindolin-2-yl]-N-thiazol-2-yl-acetamide; dihydrochloride (48 mg, 74.47 μmol) and 2-[4-[4-[[(3S)-2,6-dioxo-3-piperidyl]amino]-2-fluoro-phenyl]-1-piperidyl]acetic acid, trifluoroacetic acid salt (42.66 mg, 89.36 μmol) were mixed in DMF (0.5 mL), the reaction mixture was cooled to 0° C. N,N-Diisopropylethylamine (57.74 mg, 446.80 μmol, 77.82 μL) was added to the reaction mixture, and HATU (36.81 mg, 96.81 μmol) was added, and the reaction mixture was stirred for 1 h at 0° C. The reaction mixture was acidified with 4-5 drops of TFA, and injected directly on a RP C18 column (50 g C18) for purification (5% to 100% acetonitrile (+0.1% TFA) in water (+0.1% TFA) over 12 minutes). The pure fractions were neutralized with aqueous aqueous NaHCO₃ (ca. 60 mL), extracted twice with 1:4 isopropanol:chloroform mixture. The organic layer was dried over Na₂SO₄, filtered, and evaporated under reduced pressure to afford a solid. The solid was purified by silica gel chromatography (0% to 20% methanol in dichloromethane). The desired fractions were evaporated under reduced pressure. The residue was dissolved in dichloromethane, transferred to a 8 mL vial, and evaporated under reduced pressure. 1 mL water+1 mL acetonitrile were added, and the mixture was thoroughly sonicated, vortexed and sonicated again. The suspension was frozen and lyophilized to afford Compound 58 (19.2 mg, 20.73 μmol, 27.84% yield). LCMS (ESI+): 917.2 (M+H), 1H NMR (400 MHz, DMSO-d₆) δ 12.53 (s, 1H), 10.78 (s, 1H), 7.84 (s, 1H), 7.81 (d, J=10.4 Hz, 1H), 7.77 (d, J=7.9 Hz, 2H), 7.62 (s, 1H), 7.49 (d, J=3.6 Hz, 1H), 7.45 (d, J=7.9 Hz, 2H), 7.26 (d, J=3.6 Hz, 1H), 6.98 (t, J=8.7 Hz, 1H), 6.50-6.40 (m, 2H), 6.16 (s, 1H), 5.99 (d, J=7.7 Hz, 1H), 4.84 (d, J=17.8 Hz, 1H), 4.37-4.21 (m, 2H), 4.00 (td, J=14.1, 11.6, 6.5 Hz, 2H), 3.58 (d, J=13.2 Hz, 5H), 3.47 (s, 3H), 3.14 (s, 2H), 2.88 (d, J=10.9 Hz, 3H), 2.83-2.41 (m, 4H), 2.34 (s, 3H), 2.16-1.99 (m, 4H), 1.87 (ddd, J=25.0, 12.1, 4.1 Hz, 1H), 1.72-1.51 (m, 4H).

### Example 31. Synthesis of 2-(6,7-dihydro-5H-pyrrolo[1,2-c]imidazol-1-yl)-2-[6-[4-[[4-[2-[4-[4-[[(3S)-2,6-dioxo-3-piperidyl]amino]-2-fluoro-phenyl]-1-piperidyl]-2-oxo-ethyl]piperazin-1-yl]methyl]phenyl]-4-fluoro-1-oxo-isoindolin-2-yl]-N-thiazol-2-yl-acetamide, (Compound 59)

### Step 1: tert-butyl 2-[4-[[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]methyl]piperazin-1-yl]acetate

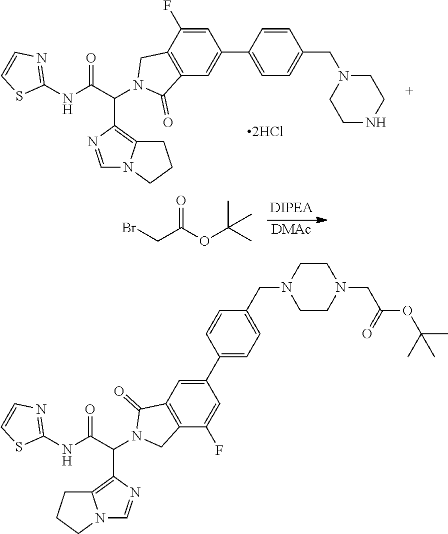

To a stirred solution of 2-(6,7-dihydro-5H-pyrrolo[1,2-c]imidazol-1-yl)-2-[4-fluoro-1-oxo-6-[4-(piperazin-1-ylmethyl)phenyl]isoindolin-2-yl]-N-thiazol-2-yl-acetamide dihydrochloride (109.5 mg, 160.78 μmol) in DMAc (1 mL) was added N,N-diisopropylethylamine (72.73 mg, 562.73 μmol, 98.02 μL) and tert-butyl 2-bromoacetate (34.50 mg, 176.86 μmol, 25.94 The reaction mixture was stirred at ambient temperature. After completion, the reaction mixture was diluted with chloroform/isopropanol (4:1) and aqueous sodium bicarbonate was added. The organic layers were separated, washed with brine, dried over Na₂SO₄, filtered and concentrated under reduced pressure. The mixture was purified by silica gel column chromatography (dichloromethane:methanol) to give tert-butyl 2-[4-[[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]methyl]piperazin-1-yl]acetate (64 mg, 93.32 μmol, 58% yield). LCMS (ESI+): 686.3 (M+H)

### Step 3: N2-(6,7-dihydro-5H-pyrrolo[1,2-c]imidazol-1-yl)-2-[6-[4-[[4-[2-[4-[4-[[(3S)-2,6-dioxo-3-piperidyl]amino]-2-fluoro-phenyl]-1-piperidyl]-2-oxo-ethyl]piperazin-1-yl]methyl]phenyl]-4-fluoro-1-oxo-isoindolin-2-yl]-N-thiazol-2-yl-acetamide

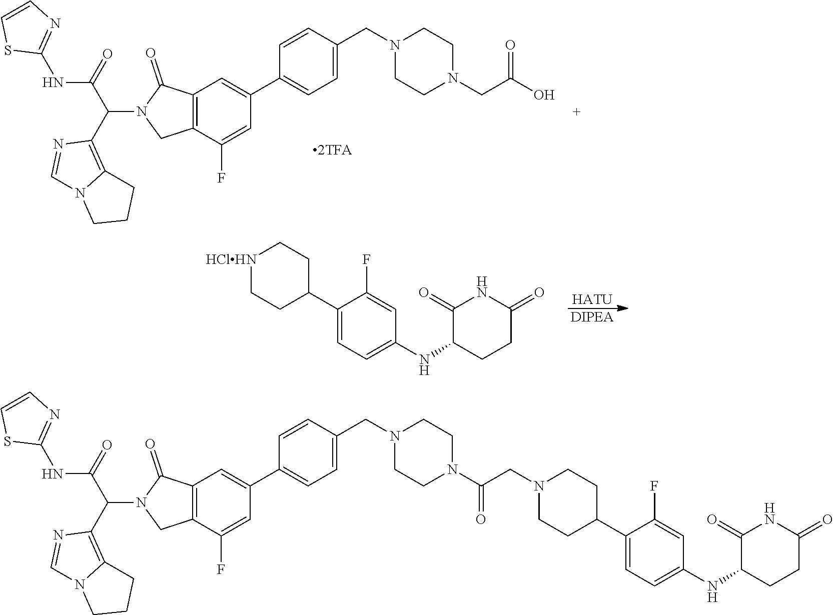

2-[4-[[4-[2-[1-(6,7-Dihydro-5H-pyrrolo[1,2-c]imidazol-1-yl)-2-oxo-2-(thiazol-2-ylamino)ethyl]-7-fluoro-3-oxo-isoindolin-5-yl]phenyl]methyl]piperazin-1-yl]acetic acid, trifluoroacetic acid salt (78.79 mg, 91.86 μmol) and (3S)-3-[3-fluoro-4-(4-piperidyl)anilino]piperidine-2,6-dione; hydrochloride (37.68 mg, 110.23 μmol) were mixed in DMF and cooled to 0° C. N,N-Diisopropylethylamine (83.10 mg, 643.00 μmol, 112.00 μL) was added to the reaction mixture, and HATU (45.40 mg, 119.41 μmol) was added, and the reaction mixture was stirred for 1 h at 0° C. The reaction mixture was acidified with 4-5 drops of TFA and injected directly on a RP C18 column (50 g C18) for purification (5% to 100% acetonitrile in water+0.1% TFA over 12 minutes). The desired fractions were neutralized with aqueous NaHCO₃ (ca. 60 mL), extracted with 1:4 isopropanol:chloroform mixture X2. The organic layer was dried over Na₂SO₄, filtered, and evaporated under reduced pressure to afford a solid. The solid was purified by silica gel column chromatography (0% to 20% methanol in dichloromethane). The desired fractions were evaporated under reduced pressure. The residue was dissolved in dichloromethane, transferred to a 8 mL vial, and evaporated under reduced pressure. 1 mL water+1 mL acetonitrile were added, and the mixture was thoroughly sonicated, vortexed and sonicated again. The suspension was frozen and lyophilized to afford Compound 59 (45 mg, 48.58 μmol, 52.89% yield). LCMS (ESI+): 917.3 (M+H), ¹H-NMR (400 MHz, DMSO-d₆) δ 12.52 (s, 1H), 10.79 (s, 1H), 7.85-7.79 (m, 2H), 7.76 (d, J=7.9 Hz, 2H), 7.61 (s, 1H), 7.49 (d, J=3.6 Hz, 1H), 7.42 (d, J=7.9 Hz, 2H), 7.26 (d, J=3.6 Hz, 1H), 6.95 (t, J=8.7 Hz, 1H), 6.48 (s, 1H), 6.45 (d, J=6.3 Hz, 1H), 6.16 (s, 1H), 6.03 (d, J=7.7 Hz, 1H), 4.83 (d, J=17.8 Hz, 1H), 4.48 (d, J=12.9 Hz, 1H), 4.37-4.20 (m, 2H), 4.16 (d, J=12.7 Hz, 1H), 4.07-3.93 (m, 2H), 3.59-3.46 (m, 3H), 3.44-3.23 (m, 2H), 3.13-2.98 (m, 2H), 2.88 (t, J=10.8 Hz, 2H), 2.82-2.36 (m, 10H), 2.14-2.03 (m, 2H), 1.93-1.81 (m, 1H), 1.77-1.57 (m, 4H), 1.42 (m, 1H).

### Example 32. Synthesis of 2-(6,7-dihydro-5H-pyrrolo[1,2-c]imidazol-1-yl)-2-[6-[4-[[1-[2-[4-[4-[[(3S)-2,6-dioxo-3-piperidyl]amino]-2-fluoro-phenyl]-1-piperidyl]acetyl]-4-piperidyl]methyl]phenyl]-4-fluoro-1-oxo-isoindolin-2-yl]-N-thiazol-2-yl-acetamide, (Compound 60)

### Step 1: tert-butyl 4-[[4-[2-[1-(6,7-dihydro-5H-pyrrolo[1,2-c]imidazol-1-yl)-2-ethoxy-2-oxo-ethyl]-7-fluoro-3-oxo-isoindolin-5-yl]phenyl]methyl]piperidine-1-carboxylate

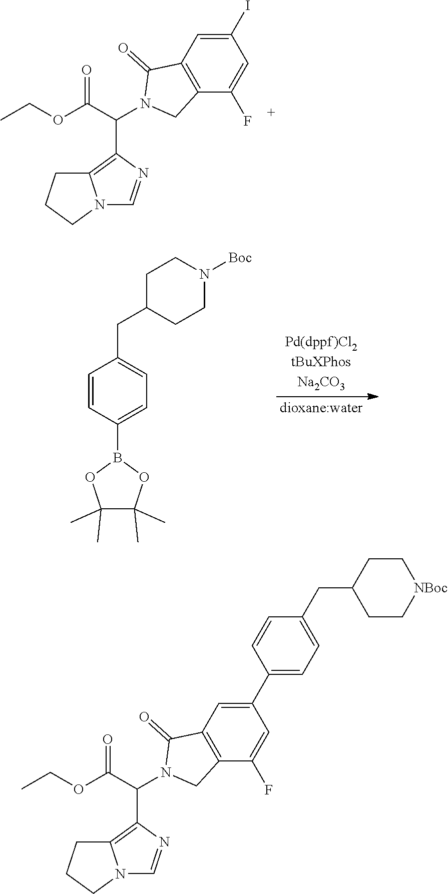 [SMILES: CCOC(=O)C(c1ncn2c1CCC2)N1Cc2c(F)cc(I)cc2C1=O ; CC(C)(C)OC(=O)N1CCC(Cc2ccc(B3OC(C)(C)C(C)(C)O3)cc2)CC1 ; CCOC(=O)C(c1ncn2c1CCC2)N1Cc2c(F)cc(-c3ccc(CC4CCN(C(=O)OC(C)(C)C)CC4)cc3)cc2C1=O]

Ethyl 2-(6,7-dihydro-5H-pyrrolo[1,2-c]imidazol-1-yl)-2-(4-fluoro-6-iodo-1-oxo-isoindolin-2-yl)acetate (400 mg, 852.43 μmol) and tert-butyl 4-[[4-(4,4,5,5-tetramethyl-1,3,2-dioxaborolan-2-yl)phenyl]methyl]piperidine-1-carboxylate (461.86 mg, 1.15 mmol) were dissolved in dioxane (4.8 mL) and tBuXPhos (53.81 mg, 85.24 μmol) was added, followed by Sodium carbonate (198.77 mg, 1.88 mmol, 78.56 μL) dissolved in Water (1.2 mL). The mixture was degassed with argon and Pd(dppf)Cl₂ (31.18 mg, 42.62 μmol) was added. The reaction was sealed and heated at 80° C. on a heating block for 2 h. The mixture was concentrated and purified by silica gel chromatography on (0-100% ethyl acetate in hexane). The desired fractions were concentrated and re-purified by silica gel chromatography (0-20% methanol in ethyl acetate) to give tert-butyl 4-[[4-[2-[1-(6,7-dihydro-5H-pyrrolo[1,2-c]imidazol-1-yl)-2-ethoxy-2-oxo-ethyl]-7-fluoro-3-oxo-isoindolin-5-yl]phenyl]methyl]piperidine-1-carboxylate (380 mg, 616.16 μmol, 72.28% yield). LCMS (ESI+): 617.3 (M+H)

### Step 2: [2-[6-[4-[(1-tert-butoxycarbonyl-4-piperidyl)methyl]phenyl]-4-fluoro-1-oxo-isoindolin-2-yl]-2-(6,7-dihydro-5H-pyrrolo[1,2-c]imidazol-1-yl)acetyl]oxylithium

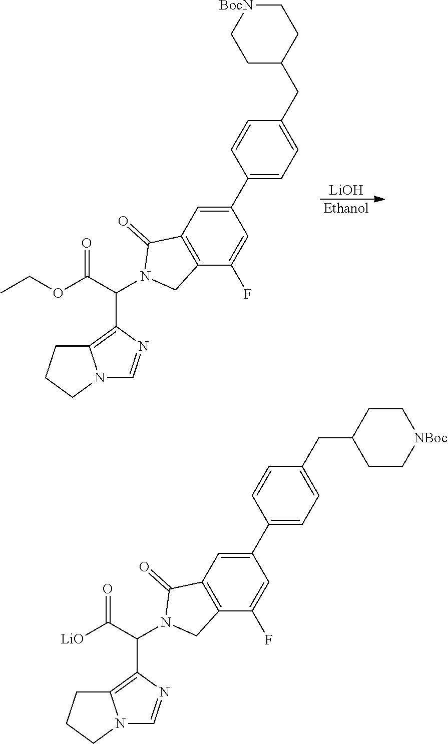

To a solution of tert-butyl 4-[[4-[2-[1-(6,7-dihydro-5H-pyrrolo[1,2-c]imidazol-1-yl)-2-ethoxy-2-oxo-ethyl]-7-fluoro-3-oxo-isoindolin-5-yl]phenyl]methyl]piperidine-1-carboxylate (380 mg, 616.16 μmol) in Ethanol (2.8 mL) was added Lithium hydroxide (1 M aqueous solution, 678 μmol, 678 μL) and stirred at ambient temperature. The reaction mixture was evaporated to dryness to afford [2-[6-[4-[(1-tert-butoxycarbonyl-4-piperidyl)methyl]phenyl]-4-fluoro-1-oxo-isoindolin-2-yl]-2-(6,7-dihydro-5H-pyrrolo[1,2-c]imidazol-1-yl)acetyl]oxylithium (366 mg, 616 μmol) as a yellow solid in quantitative yield. LCMS (ESI+): 589.2 (M+H)

### Step 3: tert-butyl 4-[[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]methyl]piperidine-1-carboxylate

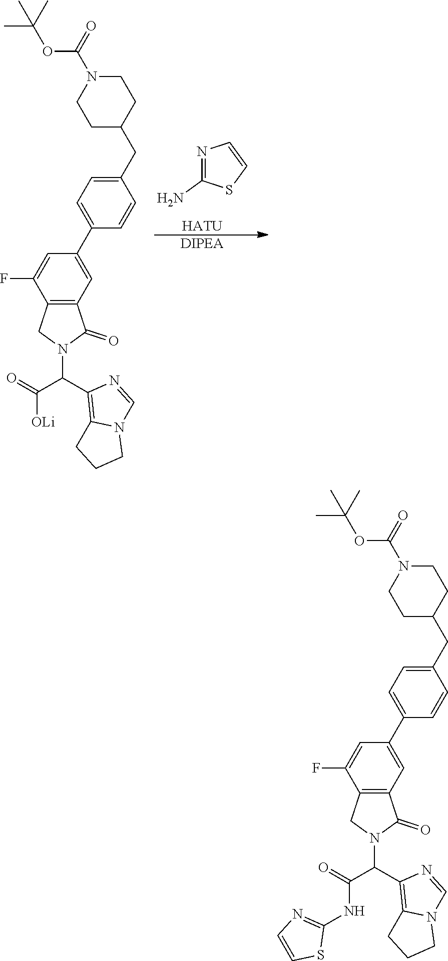

[2-[6-[4-[(1-tert-butoxycarbonyl-4-piperidyl)methyl]phenyl]-4-fluoro-1-oxo-isoindolin-2-yl]-2-(6,7-dihydro-5H-pyrrolo[1,2-c]imidazol-1-yl)acetyl]oxylithium (366.37 mg, 616.16 μmol) and thiazol-2-amine (64.79 mg, 646.97 μmol) were mixed in DMF, the reaction mixture was cooled to 0° C. N,N-Diisopropylethylamine (318.53 mg, 2.46 mmol, 429.29 μL) was added to the reaction mixture, and HATU (304.57 mg, 801.01 μmol) was added, and the reaction mixture was stirred for 30 min at 0° C. The reaction mixture was quenched with saturated NaHCO₃-solution and extracted with ethyl acetate. The organic layers were washed with water and brine, dried over Na₂SO₄, filtered and concentrated. The residue was purified by silica gel chromatography (0-10% methanol in dichloromethane) to afford tert-butyl 4-[[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]methyl]piperidine-1-carboxylate (250 mg, 372.69 μmol, 60.49% yield) LCMS (ESI+): 671.2 (M+H).

### Step 4: 2-(6,7-dihydro-5H-pyrrolo[1,2-c]imidazol-1-yl)-2-[4-fluoro-1-oxo-6-[4-(4-piperidylmethyl)phenyl]isoindolin-2-yl]-N-thiazol-2-yl-acetamide hydrochloride

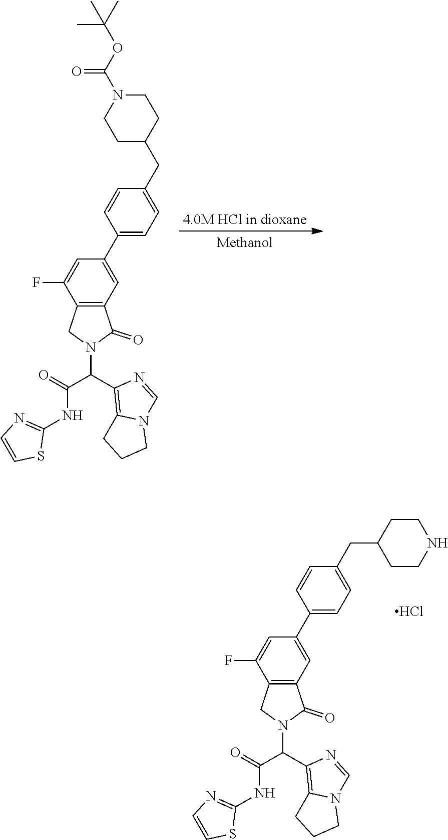 [SMILES: CC(C)(C)OC(=O)N1CCC(Cc2ccc(-c3cc(F)c4c(c3)C(=O)N(C(C(=O)Nc3nccs3)c3ncn5c3CCC5)C4)cc2)CC1 ; O=C(Nc1nccs1)C(c1ncn2c1CCC2)N1Cc2c(F)cc(-c3ccc(CC4CCNCC4)cc3)cc2C1=O ; Cl]

tert-Butyl 4-[[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]methyl]piperidine-1-carboxylate (250 mg, 372.69 μmol) was dissolved in methanol (3 mL) and Hydrogen chloride solution (4.0M in dioxane, 652.67 μL, 2.62 mmol) was added. The reaction mixture was heated at 40° C. for 4 hours. The volatiles were evaporated under reduce pressure. The material was submitted to high vacuum, frozen to −78° C. and thawed to afford a dense solid. The crude material was purified by ISCO column (dichloromethane:methanol=100:0-->50:50) to give 2-(6,7-dihydro-5H-pyrrolo[1,2-c]imidazol-1-yl)-2-[4-fluoro-1-oxo-6-[4-(4-piperidylmethyl)phenyl]isoindolin-2-yl]-N-thiazol-2-yl-acetamide hydrochloride (157 mg, 258.59 μmol, 69.38% yield). LCMS (ESI+): 571.2 (M+H)

### Step 5: 2-(6,7-dihydro-5H-pyrrolo[1,2-c]imidazol-1-yl)-2-[6-[4-[[1-[2-[4-[4-[[(3S)-2,6-dioxo-3-piperidyl]amino]-2-fluoro-phenyl]-1-piperidyl]acetyl]-4-piperidyl]methyl]phenyl]-4-fluoro-1-oxo-isoindolin-2-yl]-N-thiazol-2-yl-acetamide

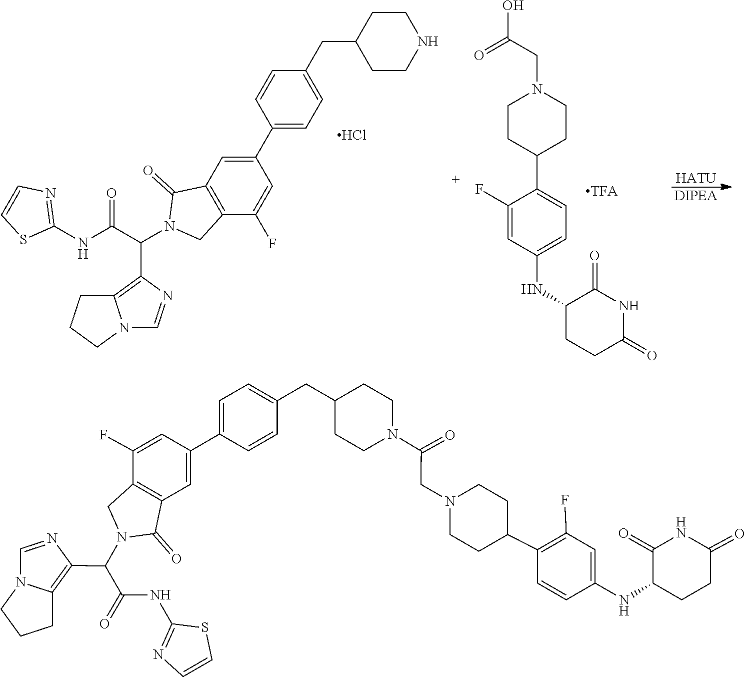 [SMILES: O=C(Nc1nccs1)C(c1ncn2c1CCC2)N1Cc2c(F)cc(-c3ccc(CC4CCNCC4)cc3)cc2C1=O ; Cl ; O=C(O)CN1CCC(c2ccc(N[C@H]3CCC(=O)NC3=O)cc2F)CC1 ; "HATU" ; "DIPEA" ; O=C1CC[C@H](Nc2ccc(C3CCN(CC(=O)N4CCC(Cc5ccc(-c6cc(F)c7c(c6)C(=O)N(C(C(=O)Nc6nccs6)c6ncn8c6CCC8)C7)cc5)CC4)CC3)c(F)c2)C(=O)N1]

2-(6,7-dihydro-5H-pyrrolo[1,2-c]imidazol-1-yl)-2-[4-fluoro-1-oxo-6-[4-(4-piperidylmethyl)phenyl]isoindolin-2-yl]-N-thiazol-2-yl-acetamide; hydrochloride (47 mg, 77.41 μmol) and 2-[4-[4-[[(3S)-2,6-dioxo-3-piperidyl]amino]-2-fluoro-phenyl]-1-piperidyl]acetic acid, trifluoroacetic acid salt(44.35 mg, 92.89 μmol) were mixed in DMF and cooled to 0° C. N,N-Diisopropylethylamine (50.02 mg, 387.06 μmol, 67.42 μL) was added to the reaction mixture, and HATU (38.26 mg, 100.64 μmol) was added, and the reaction mixture was stirred for 1 h at 0° C. The reaction mixture was acidified with 4-5 drops of TFA and injected directly on a C18 column (50 g C18) for purification (5% to 100% acetonitrile in water+0.1% TFA). The desired fractions were neutralized with aqueous NaHCO₃ (ca. 60 mL), extracted twice with a 1:4 isopropanol:chloroform mixture. The organic layer was dried over Na₂SO₄, filtered, and evaporated under reduced pressure to afford a solid. The solid was purified by silica gel chromatography (0% to 20% methanol in dichloromethane). The desired fractions were evaporated under reduced pressure. The crude residue was dissolved in dichloromethane, transferred to an 8 mL vial, and evaporated under reduced pressure. water (1 mL) and acetonitrile (1 mL) were added, and the mixture was thoroughly sonicated, vortexed and sonicated again. The suspension was frozen and lyophilized to afford Compound 60 (44.9 mg, 48.52 μmol, 62.68% yield). LCMS (ESI+): 916.3 (M+H), ¹H NMR (400 MHz, DMSO-d₆) δ 12.52 (s, 1H), 10.78 (s, 1H), 7.82 (s, 1H), 7.78 (d, J=10.4 Hz, 1H), 7.72 (d, J=7.9 Hz, 2H), 7.61 (s, 1H), 7.49 (d, J=3.6 Hz, 1H), 7.32 (d, J=8.0 Hz, 2H), 7.26 (d, J=3.6 Hz, 1H), 6.98 (t, J=8.6 Hz, 1H), 6.50-6.40 (m, 2H), 6.16 (s, 1H), 5.99 (d, J=7.8 Hz, 1H), 4.83 (d, J=17.8 Hz, 1H), 4.36-4.29 (m, 2H), 4.25 (d, J=17.8 Hz, 1H), 4.12-3.93 (m, 3H), 3.21 (d, J=13.1 Hz, 1H), 3.10-2.84 (m, 4H), 2.82-2.66 (m, 3H), 2.65-2.54 (m, 6H), 2.07 (d, J=10.7 Hz, 3H), 1.94-1.78 (m, 2H), 1.75-1.54 (m, 6H), 1.30-1.13 (m, 2H), 1.02 (m, 1H).

### Example 33. Synthesis of (2RS)-2-[4,7-Dichloro-6-[4-[4-[2-[4-[4-[[(3RS)-2,6-dioxo-3-piperidyl]amino]phenyl]-1-piperidyl]acetyl]piperazin-1-yl]phenyl]indazol-2-yl]-2-(6,7-dihydro-5H-pyrrolo[1,2-c]imidazol-1-yl)-N-thiazol-2-yl-acetamide (Compound 61)

### Step 1: 4-Bromo-3,6-dichloro-2-fluorobenzaldehyde

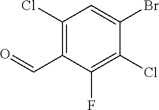 [SMILES: O=Cc1c(Cl)cc(Br)c(Cl)c1F]

A solution of 1-bromo-2,5-dichloro-3-fluorobenzene (CAS 202865-57-4) (9.414 g, 38.6 mmol) in tetrahydrofuran (70 ml) was cooled in a dry ice/acetone bath. LDA, 2 mol/l in THF (21.2 ml, 42.5 mmol, Eq: 1.1) was added and the mixture was stirred at −75° C. for 20 min. N,N-dimethylformamide (2.82 g, 2.99 ml, 38.6 mmol, Eq: 1) was added dropwise and stirred for 1 hour. A solution of acetic acid in diethylether (1:1, 10 ml) was added. The mixture was allowed to warm to room temperature. Water was added and the mixture was extracted with ethylacetate. The organic layers were washed with water, dried (MgSO₄), filtered and concentrated in vacuo to give crude 4-bromo-3,6-dichloro-2-fluorobenzaldehyde (11.3 g, 41.6 mmol, >100%) as a light yellow solid. The compound was used for the next step without further purification.

### Step 2: 6-Bromo-4,7-dichloro-1H-indazole

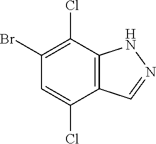 [SMILES: Clc1cc(Br)c(Cl)c2[nH]ncc12]

To a solution of 4-bromo-3,6-dichloro-2-fluorobenzaldehyde (Example 33, step 1) (10.5 g, 38.6 mmol) in Dioxane (50 ml) was added hydrazine hydrate (CAS 10217-52-4) (3.86 g, 3.78 ml, 77.2 mmol, Eq: 2.0). The mixture was stirred at room temperature for 3 days. Hydrazine hydrate (3.86 g, 3.78 ml, 77.2 mmol, Eq: 2.0) was added and the mixture was warmed to 70° C. for 7 hours. After cooling to room temperature water was added and the precipitated solid was collected by filtration. To the solid was added a small amount of acetonitrile and stirred for 2 hours. The solid was collected by filtration, washed with a small amount of acetonitrile and dried to give 6-bromo-4,7-dichloro-1H-indazole (7.84 g, 29.5 mmol, 76.4%) as an off-white solid. MS: m/e=267.0 ([M+H]⁺).

### Step 3: Ethyl 2-(6-bromo-4,7-dichloro-2H-indazol-2-yl)acetate

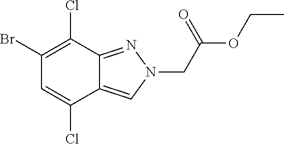 [SMILES: CCOC(=O)Cn1cc2c(Cl)cc(Br)c(Cl)c2n1]

A mixture of 6-bromo-4,7-dichloro-1H-indazole (Example 33, step 2) (7.84 g, 29.5 mmol) and ethyl 2-bromoacetate (CAS 105-36-2) (9.85 g, 6.53 ml, 59 mmol, Eq: 2) in N,N-dimethylacetamide (11.5 ml) was heated to 100° C. for 25 hours. After cooling to room temperature, ice is added and the precipitated solid is collected by filtration and washed with water. The crude was dissolved in boiling ethanol. After cooling the solid was filtered, washed with ethanol and dried to afford ethyl 2-(6-bromo-4,7-dichloro-2H-indazol-2-yl)acetate (7.511 g, 21.3 mmol, 70.9%) MS: m/e=267.0 ([M+H]⁺).

### Step 4: tert-Butyl (2S)-2-[(2RS)-2-(6-bromo-4,7-chloro-indazol-2-yl)-3-ethoxy-3-oxo-propanoyl]pyrrolidine-1-carboxylate

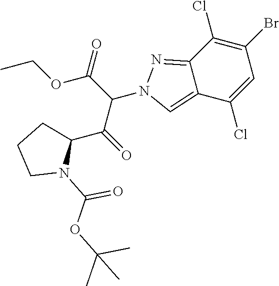 [SMILES: CCOC(=O)C(C(=O)[C@@H]1CCCN1C(=O)OC(C)(C)C)n1cc2c(Cl)cc(Br)c(Cl)c2n1]

A solution of (tert-butoxycarbonyl)-L-proline (CAS 15761-39-4) (4.93 g, 22.9 mmol, Eq: 1.55) in Tetrahydrofuran (25 ml) was cooled in an ice bath. Carbonyldiimidazole (3.71 g, 22.9 mmol, Eq: 1.55) was added. The cooling bath was removed and the mixture was stirred for 3h to give solution A. A solution of ethyl 2-(6-bromo-4,7-dichloro-2H-indazol-2-yl)acetate (Example 33, step 3) (5.2 g, 14.8 mmol, Eq: 1) in Tetrahydrofuran (7.5 ml) was cooled to −75° C. LDA, 2 mol/l in THF (11.4 ml, 22.9 mmol, Eq: 1.55) was added dropwise within 5 min. The mixture was stirred for 30 min at −75° C. Solution A was added dropwise within 5 min. The mixture was allowed to warm to room temperature in the cooling bath overnight. After addition of saturated aqueous NH₄Cl-solution, the mixture was extracted twice with ethylacetate. The organic layers were washed with water, combined, dried over sodium sulphate and concentrated to dryness to give tert-butyl (2S)-2-[(2RS)-2-(6-bromo-4,7-chloro-indazol-2-yl)-3-ethoxy-3-oxo-propanoyl]pyrrolidine-1-carboxylate (10.06 g >100%) which was used for the next step without further purification. MS: m/e=550.2 ([M+H]⁺).

### Step 5: Ethyl (2RS)-2-(6-bromo-4,7-dichloro-indazol-2-yl)-2-(3-thioxo-2,5,6,7-tetrahydropyrrolo[1,2-c]imidazol-1-yl)acetate

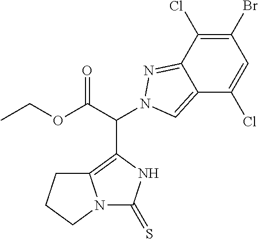 [SMILES: CCOC(=O)C(c1[nH]c(=S)n2c1CCC2)n1cc2c(Cl)cc(Br)c(Cl)c2n1]

A solution of tert-butyl (2S)-2-[(2RS)-2-(6-bromo-6,7-chloro-indazol-2-yl)-3-ethoxy-3-oxo-propanoyl]pyrrolidine-1-carboxylate (Example 33, step 4) (10 g, 18.2 mmol) in HCl, 4M in dioxane (31.9 ml) was stirred for 1 hour at room temperature. The mixture was concentrated to dryness. The residue was dissolved in ethanol (87.5 ml), potassium thiocyanate (2.35 g, 24.2 mmol, Eq: 1.33) and HCl, 0.5 M in ethanol (36.4 ml, 18.2 mmol, Eq: 1) were added and stirred for 36 hours. Water was added and the mixture was extracted with ethylacetate. The organic layers were washed with brine, dried over MgSO₄, filtered, concentrated and dried to give ethyl (2RS)-2-(6-bromo-4,7-dichloro-indazol-2-yl)-2-(3-thioxo-2,5,6,7-tetrahydropyrrolo[1,2-c]imidazol-1-yl)acetate (8.534 g, 17.4 mmol, 95.6%) which was used for the next step without further purification. MS: m/e=491.1 ([M+H]⁺).

### Step 6: Ethyl (2RS)-2-(6-bromo-4,7-dichloro-indazol-2-yl)-2-(6,7-dihydro-5H-pyrrolo[1,2-c]imidazol-1-yl)acetate

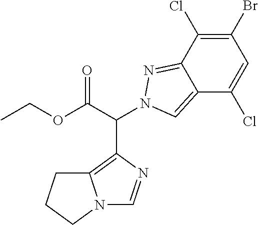 [SMILES: CCOC(=O)C(c1ncn2c1CCC2)n1cc2c(Cl)cc(Br)c(Cl)c2n1]

A solution of ethyl (2RS)-2-(6-bromo-4,7-dichloro-indazol-2-yl)-2-(3-thioxo-2,5,6,7-tetrahydropyrrolo[1,2-c]imidazol-1-yl)acetate (Example 33, step 5) (8.53 g, 17.4 mmol) in AcOH (32.2 ml) at 40° C. After cooling to room temperature, hydrogen peroxide 35% (6.76 g, 6.09 ml, 69.6 mmol, Eq: 4) was added dropwise. The reaction mixture was stirred for 1 hour at room temperature. The excess of hydrogen peroxide was destroyed by addition of saturated sodium sulfit solution. After addition of some water (just enough to dissolve all salts) and ethylacetate the mixture was brought to pH 9 by careful addition of solid sodium carbonate. The mixture was extracted with ethylacetate. The organic layers were washed with water, dried over sodium sulphate and concentrated. The crude product was purified by chromatography (SiO₂, 0-100% ethylacetate in heptane) to give ethyl (2RS)-2-(6-bromo-4,7-dichloro-indazol-2-yl)-2-(6,7-dihydro-5H-pyrrolo[1,2-c]imidazol-1-yl)acetate (3.21 g, 6.67 mmol, 40.3%) as a light yellow solid. MS: m/e=457.1 ([M+H]⁺).

### Step 7: tert-Butyl 4-[4-[4,7-dichloro-2-[(1RS)-1-(6,7-dihydro-5H-pyrrolo[1,2-c]imidazol-1-yl)-2-ethoxy-2-oxo-ethyl]indazol-6-yl]phenyl]piperazine-1-carboxylate

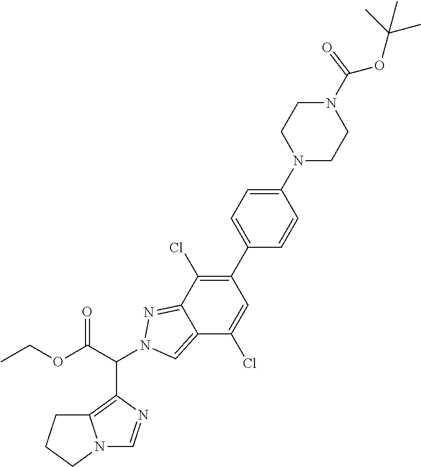 [SMILES: CCOC(=O)C(c1ncn2c1CCC2)n1cc2c(Cl)cc(-c3ccc(N4CCN(C(=O)OC(C)(C)C)CC4)cc3)c(Cl)c2n1]

(2RS)-2-(6-Bromo-4,7-dichloro-indazol-2-yl)-2-(6,7-dihydro-5H-pyrrolo[1,2-c]imidazol-1-yl)acetate (Example 33, step 6) (200 mg, 437 μmol) and (4-(4-(tert-butoxycarbonyl)piperazin-1-yl)phenyl)boronic acid (CAS 457613-78-4) (401 mg, 1.31 mmol, Eq: 3) were mixed with toluene (5.3 ml), degassed by bubbling argon through the mixture under ultra sonic treatment. [1,1′-Bis(diphenylphosphino)ferrocene]dichloropalladium(II) (31.9 mg, 43.7 μmol, Eq:0.1) was added and the mixture was stirred for 40 min at 115° C. in a sealed tube. The mixture was cooled to room temperature, diluted with ethylacetate, washed with half concentrated sodium carbonate solution, dried over sodium sulphate and concentrated. The crude material was purified by flash chromatography (SiO₂, 0% to 100% ethylacetate:methanol 3:2 in ethylacetate) to give tert-butyl 4-[4-[4,7-dichloro-2-[(1RS)-1-(6,7-dihydro-5H-pyrrolo[1,2-c]imidazol-1-yl)-2-ethoxy-2-oxo-ethyl]indazol-6-yl]phenyl]piperazine-1-carboxylate (191.5 mg, 29.9 mmol, 63.1%) as a light brown solid. MS: m/e=639.5 ([M+H]⁺).

### Step 8: tert-Butyl 4-[4-[4,7-dichloro-2-[(1RS)-1-(6,7-dihydro-5H-pyrrolo[1,2-c]imidazol-1-yl)-2-oxo-2-(thiazol-2-ylamino)ethyl]indazol-6-yl]phenyl]piperazine-1-carboxylate

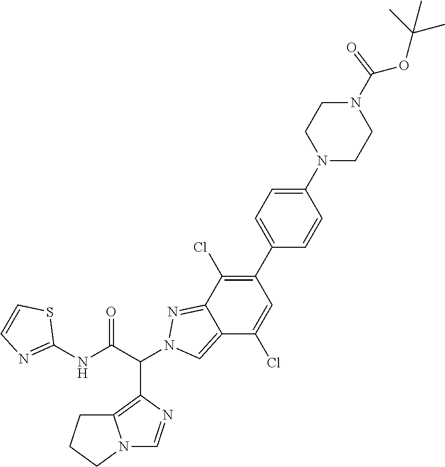 [SMILES: CC(C)(C)OC(=O)N1CCN(c2ccc(-c3cc(Cl)c4cn(C(C(=O)Nc5nccs5)c5ncn6c5CCC6)nc4c3Cl)cc2)CC1]

tert-Butyl 4-[4-[4,7-dichloro-2-[(1RS)-1-(6,7-dihydro-5H-pyrrolo[1,2-c]imidazol-1-yl)-2-ethoxy-2-oxo-ethyl]indazol-6-yl]phenyl]piperazine-1-carboxylate (Example 33, step 7) (190 mg, 0.297 mmol) was dissolved in 3 ml of THF. LiOH (1M in water) (0.45 ml, 0.446 mmol, 1.5 equiv.) was added. The reaction mixture was stirred at room temperature for 30 minutes. The reaction mixture was concentrated in vacuo. The residue was dissolved in 3 ml of N,N-dimethylformamide. Thiazol-2-amine (36 mg, 0.356 mmol, 1.2 equiv.) and Hunig's base (0.156 ml, 0.89 mmol, 3 equiv.) were added followed by HATU (136 mg, 0.356 mmol, 1.2 equiv.). The mixture was stirred at room temperature for 2 hours. The reaction mixture was extracted with ethyl acetate and water. The aqueous layer was back-extracted with ethyl acetate. The organic layers were washed with water and brine. The organic layers were combined, dried over sodium sulfate, filtered and concentrated to dryness. The crude product was purified by flash chromatography on a silica gel column eluting with an ethyl acetate:methanol 100:0 to 70:30 gradient to obtain the desired product (94 mg, 44% yield) as a light brown solid, MS: m/e=691.5 ([M+H]⁺).

### Step 9: (2RS)-2-[4,7-Dichloro-6-(4-piperazin-1-ylphenyl)indazol-2-yl]-2-(6,7-dihydro-5H-pyrrolo[1,2-c]imidazol-1-yl)-N-thiazol-2-yl-acetamide hydrochloride salt

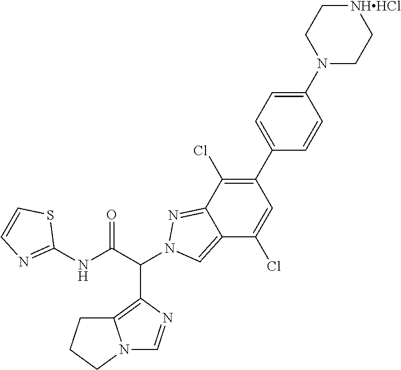

tert-Butyl 4-[4-[4,7-dichloro-2-[(1RS)-1-(6,7-dihydro-5H-pyrrolo[1,2-c]imidazol-1-yl)-2-oxo-2-(thiazol-2-ylamino)ethyl]indazol-6-yl]phenyl]piperazine-1-carboxylate (Example 33, step 9) (92 mg, 0.133 mmol) and HCl (4 M in dioxane) (1.7 ml, 6.63 mmol, 50 equiv.) were combined with 3 ml of dichloromethane and 1.8 ml of methanol. The reaction mixture was stirred at room temperature for 1 hour. The reaction mixture was concentrated to dryness and used without further purification. The desired product (93 mg, quantitative) was obtained as a light yellow solid, MS: m/e=591.4 ([M+H]⁺).

### Step 10: tert-Butyl 4-[4-[[(3RS)-2,6-dioxo-3-piperidyl]amino]phenyl]piperidine-1-carboxylate

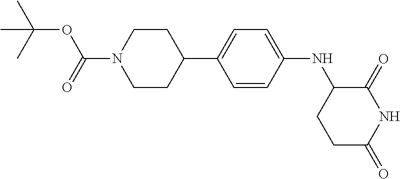 [SMILES: CC(C)(C)OC(=O)N1CCC(c2ccc(NC3CCC(=O)NC3=O)cc2)CC1]

tert-Butyl 4-(4-aminophenyl)piperidine-1-carboxylate (CAS 170011-57-1) (798 mg, 2.89 mmol) was dissolved in 10 ml of acetonitrile. Sodium bicarbonate (485 mg, 5.77 mmol, 2 equiv.) was added followed by 3-bromopiperidine-2,6-dione (CAS 62595-74-8) (610 mg, 3.18 mmol, 1.1 equiv.). The reaction mixture was stirred at 90° C. for 16 hours. The reaction mixture was cooled to room temperature, adsorbed on Isolute® and purified by flash chromatography on a silica gel column eluting with an ethyl acetate:heptane 30:70 to 100:0 gradient. The desired tert-butyl 4-[4-[[(3RS)-2,6-dioxo-3-piperidyl]amino]phenyl]piperidine-1-carboxylate (850 mg, 76% yield) was obtained as an off-white solid, MS: m/e=359.4 (([M-tBu+H]⁺).

### Step 11: (3RS)-3-[4-(4-Piperidyl)anilino]piperidine-2,6-dione hydrochloride

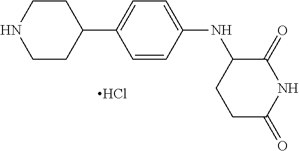 [SMILES: O=C1CCC(Nc2ccc(C3CCNCC3)cc2)C(=O)N1 ; Cl]

tert-Butyl 4-[4-[[(3RS)-2,6-dioxo-3-piperidyl]amino]phenyl]piperidine-1-carboxylate (Example 33, step 10) (850 mg, 2.19 mmol) and HCl (4 M in dioxane) (5.48 ml, 21.9 mmol, 10 equiv.) were combined with 10 ml of methanol at 0-5° C. in an ice bath. The reaction mixture was stirred at room temperature for 18 hours. The reaction mixture was concentrated to dryness and used without further purification. The desired (3RS)-3-[4-(4-piperidyl)anilino]piperidine-2,6-dione hydrochloride (818 mg, quantitative, purity=87%) was obtained as an off-white solid, MS: m/e=286.1 ([M+H]⁺).

### Step 12: tert-Butyl 2-[4-[4-[[(3RS)-2,6-dioxo-3-piperidyl]amino]phenyl]-1-piperidyl]acetate

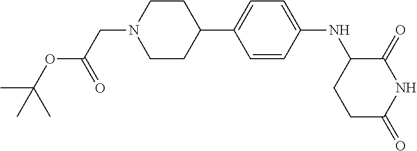 [SMILES: CC(C)(C)OC(=O)CN1CCC(c2ccc(NC3CCC(=O)NC3=O)cc2)CC1]

A mixture of (3RS)-3-[4-(4-piperidyl)anilino]piperidine-2,6-dione hydrochloride (Example 33, step 11) (200 mg, 0.618 mmol), tert-butyl 2-bromoacetate (CAS 5292-43-3) (157 mg, 0.119 ml, 0.803 mmol, 1.3 equiv.) and Hunig's base (399 mg, 0.539 ml, 3.09 mmol, 5 equiv.) in 4.0 ml of N,N-Dimethylformamide was stirred at room temperature for 2 hours. The reaction mixture was extracted with ethyl acetate and water. The aqueous layer was backextracted with ethyl acetate. The organic layers were combined, dried over sodium sulfate, filtered and concentrated to dryness. The crude product was purified by flash chromatography on a silica gel column eluting with an ethyl acetate:heptane 50:50 to 100:0 gradient. The desired tert-butyl 2-[4-[4-[[(3RS)-2,6-dioxo-3-piperidyl]amino]phenyl]-1-piperidyl]acetate (164 mg, 66% yield) was obtained as a white solid, MS: m/e=402.2 ([M+H]⁺).

### Step 13: 2-[4-[4-[[(3RS)-2,6-Dioxo-3-piperidyl]amino]phenyl]-1-piperidyl]acetic acid hydrochloride salt

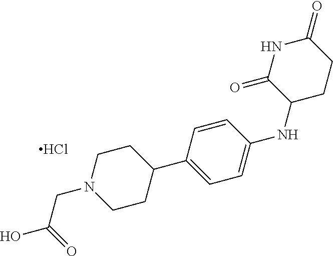 [SMILES: O=C(O)CN1CCC(c2ccc(NC3CCC(=O)NC3=O)cc2)CC1 ; Cl]

To a solution of tert-Butyl 2-[4-[4-[[(3RS)-2,6-dioxo-3-piperidyl]amino]phenyl]-1-piperidyl]acetate (Example 33, step 12) (543 mg, 1.35 mmol, Eq: 1) in ethyl acetate (8 ml) was added 4 M hydrogen chloride solution in 1,4-dioxane (6.3 g, 6 ml, 24 mmol, Eq: 17.7) at room temperature and stirring was continued over the weekend. The product was collected by filtration, washed with ethyl acetate and dried under vaccuo. The desired 2-[4-[4-[[(3RS)-2,6-Dioxo-3-piperidyl]amino]phenyl]-1-piperidyl]acetic acid (537 mg, 1.27 mmol, 93.6% yield) was obtained as light red solid. MS: m/e=346.2 ([M+H]⁺).

### Step 14: (2RS)-2-[4,7-Dichloro-6-[4-[4-[2-[4-[4-[[(3RS)-2,6-dioxo-3-piperidyl]amino]phenyl]-1-piperidyl]acetyl]piperazin-1-yl]phenyl]indazol-2-yl]-2-(6,7-dihydro-5H-pyrrolo[1,2-c]imidazol-1-yl)-N-thiazol-2-yl-acetamide

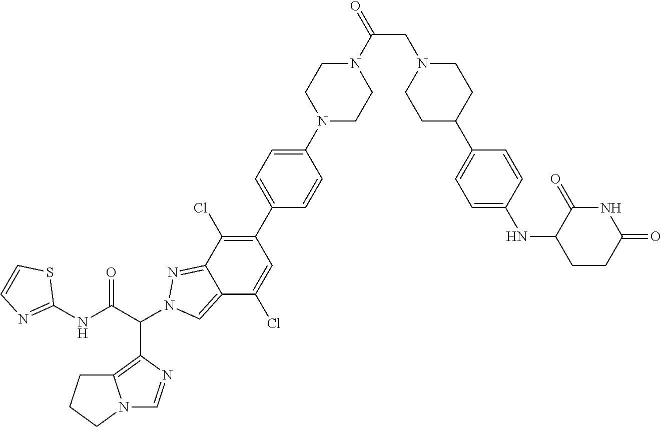 [SMILES: O=C1CCC(Nc2ccc(C3CCN(CC(=O)N4CCN(c5ccc(-c6cc(Cl)c7cn(C(C(=O)Nc8nccs8)c8ncn9c8CCC9)nc7c6Cl)cc5)CC4)CC3)cc2)C(=O)N1]

(2RS)-2-[4,7-Dichloro-6-(4-piperazin-1-ylphenyl)indazol-2-yl]-2-(6,7-dihydro-5H-pyrrolo[1,2-c]imidazol-1-yl)-N-thiazol-2-yl-acetamide hydrochloride salt (Example 33, step 9) (50 mg, 0.08 mmol) and 2-[4-[4-[[(3RS)-2,6-dioxo-3-piperidyl]amino]phenyl]-1-piperidyl]acetic acid hydrochloride salt (Example 33, step 13) (30 mg, 0.08 mmol, 1.0 equiv.) were dissolved in 0.5 ml of N,N-dimethylformamide. Hunig's base (0.07 ml, 0.4 mmol, 5 equiv.) was added followed by HATU (45 mg, 0.12 mmol, 1.5 equiv.). The reaction mixture was stirred at room temperature for 2 hours. The reaction mixture was extracted with saturated NaHCO₃-solution and three times with a mixture of dichloromethane:methanol (9:1). The organic layers were washed with water. The organic layers were combined, dried over sodium sulfate, filtered and concentrated to dryness. The crude product was purified by flash chromatography on a silica gel column eluting with a dichloromethane:methanol 100:0 to 80:20 gradient to obtain Compound 61 (17 mg, 23% yield) as an off-white solid, MS: m/e=920.5 ([M+H]⁺).

### Example 34. Synthesis of (2RS)-2-(6,7-Dihydro-5H-pyrrolo[1,2-c]imidazol-1-yl)-2-[7-fluoro-6-[6-[4-[4-oxo-4-[4-[1-oxo-2-[(3RS)-2,6-dioxo-3-piperidyl]isoindolin-4-yl]oxy-1-piperidyl]butyl]piperazin-1-yl]-3-pyridyl]indazol-2-yl]-N-thiazol-2-yl-acetamide, (Compound 62)

### Step 1: Ethyl 2-(6-bromo-7-fluoro-2H-indazol-2-yl)acetate

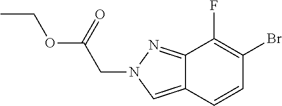 [SMILES: CCOC(=O)Cn1cc2ccc(Br)c(F)c2n1]

The title compound was obtained as a light yellow solid, MS: m/e=302.9 (M+H⁺), using chemistry similar to that described in Example 1, step 3 starting from 6-bromo-7-fluoro-1H-indazole.

### Step 2: tert-Butyl (2S)-2-[(2RS)-2-(6-bromo-7-fluoro-indazol-2-yl)-3-ethoxy-3-oxo-propanoyl]pyrrolidine-1-carboxylate

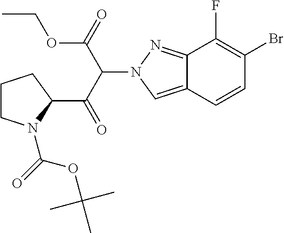 [SMILES: CCOC(=O)C(C(=O)[C@@H]1CCCN1C(=O)OC(C)(C)C)n1cc2ccc(Br)c(F)c2n1]

The title compound was obtained as a light yellow solid, MS: m/e=498.2/500.2 ([M+H]⁺) Br isotopes, using chemistry similar to that described in Example 33, step 4 starting from ethyl 2-(6-bromo-7-fluoro-2H-indazol-2-yl)acetate (Example 33, step 1).

### Step 3: Ethyl (2RS)-2-(6-bromo-7-fluoro-indazol-2-yl)-2-(3-thioxo-2,5,6,7-tetrahydropyrrolo[1,2-c]imidazol-1-yl)acetate

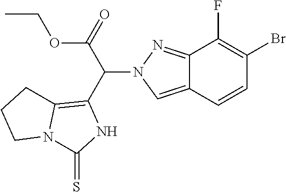 [SMILES: CCOC(=O)C(c1[nH]c(=S)n2c1CCC2)n1cc2ccc(Br)c(F)c2n1]

The title compound was obtained as a light yellow solid, MS: m/e=439.2/441.2 ([M+H]⁺ bromo isotopes) using chemistry similar to that described in Example 33, step 5 starting from tert-butyl (2S)-2-[(2RS)-2-(6-bromo-7-fluoro-indazol-2-yl)-3-ethoxy-3-oxo-propanoyl]pyrrolidine-1-carboxylate (Example 33, step 2).

### Step 4: Ethyl (2RS)-2-(6-bromo-7-fluoro-indazol-2-yl)-2-(6,7-dihydro-5H-pyrrolo[1,2-c]imidazol-1-yl)acetate

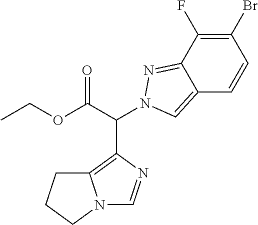 [SMILES: CCOC(=O)C(c1ncn2c1CCC2)n1cc2ccc(Br)c(F)c2n1]

The title compound was obtained as a light brown amorphous solid, MS: m/e=407.2/409.2 ([M+H]⁺) Bromo isotopes using chemistry similar to that described in Example 33, step 6 starting from (2RS)-2-(6-bromo-7-fluoro-indazol-2-yl)-2-(3-thioxo-2,5,6,7-tetrahydropyrrolo[1,2-c]imidazol-1-yl)acetate (Example 33, step 3).

### Step 5: tert-Butyl 4-[5-[7-fluoro-2-[(1RS)-1-(6,7-dihydro-5H-pyrrolo[1,2-c]imidazol-1-yl)-2-ethoxy-2-oxo-ethyl]indazol-6-yl]-2-pyridyl]piperazine-1-carboxylate

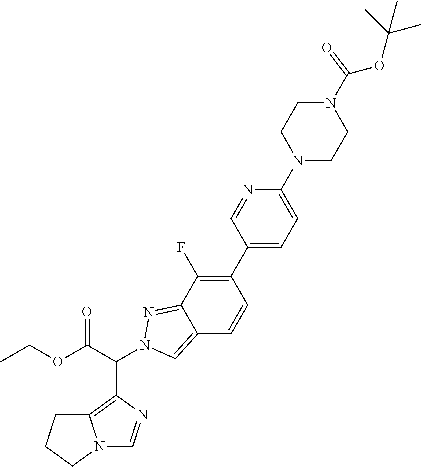 [SMILES: CCOC(=O)C(c1ncn2c1CCC2)n1cc2ccc(-c3ccc(N4CCN(C(=O)OC(C)(C)C)CC4)nc3)c(F)c2n1]

The title compound was obtained as a light brown amorphous solid, MS: m/e=590.5 ([M+H]⁺) using chemistry similar to that described in Example 33, step 7 starting from ethyl (2RS)-2-(6-bromo-7-fluoro-indazol-2-yl)-2-(6,7-dihydro-5H-pyrrolo[1,2-c]imidazol-1-yl)acetate (Example 33, step 4).

### Step 6: tert-Butyl 4-[5-[7-fluoro-2-[(1RS)-1-(6,7-dihydro-5H-pyrrolo[1,2-c]imidazol-1-yl)-2-oxo-2-(thiazol-2-ylamino)ethyl]indazol-6-yl]-2-pyridyl]piperazine-1-carboxylate

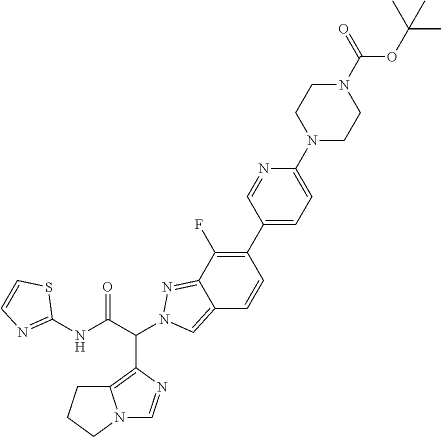 [SMILES: CC(C)(C)OC(=O)N1CCN(c2ccc(-c3ccc4cn(C(C(=O)Nc5nccs5)c5ncn6c5CCC6)nc4c3F)cn2)CC1]

The title compound was obtained as a light brown solid, MS: m/e=644.4 ([M+H]⁺), using chemistry similar to that described in Example 33, step 8 starting from tert-butyl 4-[5-[7-fluoro-2-[(1RS)-1-(6,7-dihydro-5H-pyrrolo[1,2-c]imidazol-1-yl)-2-ethoxy-2-oxo-ethyl]indazol-6-yl]-2-pyridyl]piperazine-1-carboxylate (Example 33, step 5) and thiazol-2-amine.

### Step 7: (2RS)-2-(6,7-Dihydro-5H-pyrrolo[1,2-c]imidazol-1-yl)-2-[7-fluoro-6-(6-piperazin-1-yl-3-pyridyl)indazol-2-yl]-N-thiazol-2-yl-acetamide

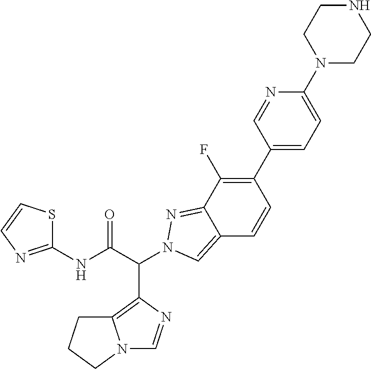 [SMILES: O=C(Nc1nccs1)C(c1ncn2c1CCC2)n1cc2ccc(-c3ccc(N4CCNCC4)nc3)c(F)c2n1]

The title compound was obtained as a light brown solid, MS: m/e=544.4 ([M+H]⁺), using chemistry similar to that described in Example 33, step 9 starting from tert-butyl 4-[5-[7-fluoro-2-[(1RS)-1-(6,7-dihydro-5H-pyrrolo[1,2-c]imidazol-1-yl)-2-oxo-2-(thiazol-2-ylamino)ethyl]indazol-6-yl]-2-pyridyl]piperazine-1-carboxylate (Example 33, step 6).

### Step 8: tert-Butyl 4-[4-[5-[7-fluoro-2-[(1RS)-1-(6,7-dihydro-5H-pyrrolo[1,2-c]imidazol-1-yl)-2-oxo-2-(thiazol-2-ylamino)ethyl]indazol-6-yl]-2-pyridyl]piperazin-1-yl]butanoate

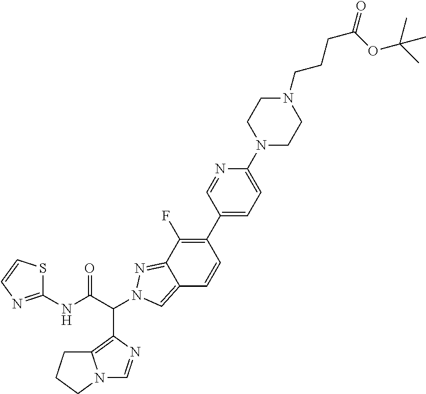 [SMILES: CC(C)(C)OC(=O)CCCN1CCN(c2ccc(-c3ccc4cn(C(C(=O)Nc5nccs5)c5ncn6c5CCC6)nc4c3F)cn2)CC1]

(2RS)-2-(6,7-Dihydro-5H-pyrrolo[1,2-c]imidazol-1-yl)-2-[7-fluoro-6-(6-piperazin-1-yl-3-pyridyl)indazol-2-yl]-N-thiazol-2-yl-acetamide (Example 33, step 7) (50 mg, 0.092 mmol) and Hunig's base (0.080 ml, 0.046 mmol, 5 equiv.) were dissolved in 1.0 ml of N,N-dimethylformamide. tert-Butyl 4-bromobutanoate (CAS 110661-91-1) (33 mg, 0.024 ml, 0.147 mmol, 1.6 equiv.) was added and the reaction mixture was stirred at 60° C. for 7 hours. The reaction mixture was extracted with water and two times with ethyl acetate. The organic layers were washed with water and brine. The organic layers were combined, dried over sodium sulfate, filtered and concentrated to dryness. The crude product was purified by flash chromatography on a silica gel column eluting with a dichloromethane:methanol 100:0 to 95:5 gradient to obtain the desired product (43 mg, 68% yield) as a light brown oil.

### Step 9: (2RS)-2-(6,7-Dihydro-5H-pyrrolo[1,2-c]imidazol-1-yl)-2-[7-fluoro-6-[6-[4-[4-oxo-4-[4-[1-oxo-2-[(3RS)-2,6-dioxo-3-piperidyl]isoindolin-4-yl]oxy-1-piperidyl]butyl]piperazin-1-yl]-3-pyridyl]indazol-2-yl]-N-thiazol-2-yl-acetamide

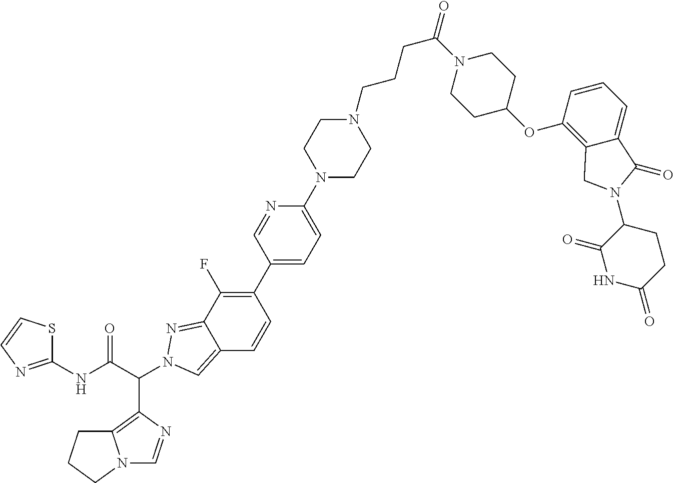 [SMILES: O=C1CCC(N2Cc3c(OC4CCN(C(=O)CCCN5CCN(c6ccc(-c7ccc8cn(C(C(=O)Nc9nccs9)c9ncn%10c9CCC%10)nc8c7F)cn6)CC5)CC4)cccc3C2=O)C(=O)N1]

tert-Butyl 4-[4-[5-[7-fluoro-2-[(1RS)-1-(6,7-dihydro-5H-pyrrolo[1,2-c]imidazol-1-yl)-2-oxo-2-(thiazol-2-ylamino)ethyl]indazol-6-yl]-2-pyridyl]piperazin-1-yl]butanoate (Example 33, step 8) (43 mg, 0.062 mmol) was dissolved in 0.3 ml of dichloromethane and trifluoroacetic acid (148 mg, 0.10 ml, 1.3 mmol, 20 equiv.) was added. The reaction mixture was stirred at room temperature for 90 minutes. The reaction mixture was concentrated to dryness. The residue and (3RS)-3-[1-oxo-4-(4-piperidyloxy)isoindolin-2-yl]piperidine-2,6-dione hydrochloride (CAS 1061605-57-9) (25 mg, 0.065 mmol, 1 equiv.) were dissolved in 0.6 ml of N,N-dimethylformamide. Hunig's base (0.11 ml, 0.62 mmol, 10 equiv.) was added followed by TBTU (22 mg, 0.069 mmol, 1.1 equiv.). The reaction mixture was stirred at room temperature for 2 hours. The reaction mixture was extracted with water and three times with a mixture of dichloromethane:methanol (9:1). The organic layers were washed with water. The organic layers were combined, dried over sodium sulfate, filtered and concentrated to dryness. The crude product was purified by flash chromatography on an amino-silica gel column eluting with a dichloromethane:methanol 100:0 to 95:5 gradient to obtain Compound 62 (30 mg, 50% yield) as an off-white foam, MS: m/e=955.6 ([M+H]⁺).

### Example 35. Synthesis of (2RS)-2-(6,7-Dihydro-5H-pyrrolo[1,2-c]imidazol-1-yl)-2-[7-fluoro-6-[6-[4-[2-[4-[4-[[(3RS)-2,6-dioxo-3-piperidyl]amino]phenyl]-1-piperidyl]acetyl]piperazin-1-yl]-3-pyridyl]indazol-2-yl]-N-thiazol-2-yl-acetamide

### Step 1: tert-Butyl 4-[4-[[(3RS)-2,6-dioxo-3-piperidyl]amino]phenyl]piperidine-1-carboxylate

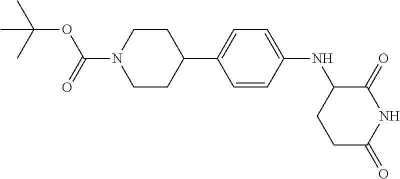 [SMILES: CC(C)(C)OC(=O)N1CCC(c2ccc(NC3CCC(=O)NC3=O)cc2)CC1]

tert-Butyl 4-(4-aminophenyl)piperidine-1-carboxylate (CAS 170011-57-1) (798 mg, 2.89 mmol) was dissolved in 10 ml of acetonitrile. Sodium bicarbonate (485 mg, 5.77 mmol, 2 equiv.) was added followed by 3-bromopiperidine-2,6-dione (CAS 62595-74-8) (610 mg, 3.18 mmol, 1.1 equiv.). The reaction mixture was stirred at 90° C. for 16 hours. The reaction mixture was cooled to room temperature, adsorbed on Isolute® and purified by flash chromatography on a silica gel column eluting with an ethyl acetate:heptane 30:70 to 100:0 gradient. The desired tert-butyl 4-[4-[[(3RS)-2,6-dioxo-3-piperidyl]amino]phenyl]piperidine-1-carboxylate (850 mg, 76% yield) was obtained as an off-white solid, MS: m/e=359.4 (([M-tBu+H]⁺).

### Step 2: (3RS)-3-[4-(4-Piperidyl)anilino]piperidine-2,6-dione hydrochloride

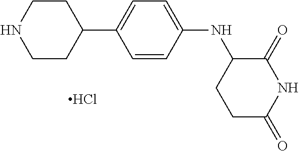 [SMILES: O=C1CCC(Nc2ccc(C3CCNCC3)cc2)C(=O)N1 ; Cl]

tert-Butyl 4-[4-[[(3RS)-2,6-dioxo-3-piperidyl]amino]phenyl]piperidine-1-carboxylate (Example 35, step 1) (850 mg, 2.19 mmol) and HCl (4 M in dioxane) (5.48 ml, 21.9 mmol, 10 equiv.) were combined with 10 ml of methanol at 0-5° C. in an ice bath. The reaction mixture was stirred at room temperature for 18 hours. The reaction mixture was concentrated to dryness and used without further purification. The desired (3RS)-3-[4-(4-piperidyl)anilino]piperidine-2,6-dione hydrochloride (818 mg, quantitative, purity=87%) was obtained as an off-white solid, MS: m/e=286.1 ([M+H]⁺).

### Step 3: tert-Butyl 2-[4-[4-[[(3RS)-2,6-dioxo-3-piperidyl]amino]phenyl]-1-piperidyl]acetate

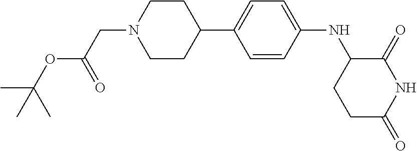 [SMILES: CC(C)(C)OC(=O)CN1CCC(c2ccc(NC3CCC(=O)NC3=O)cc2)CC1]

A mixture of (3RS)-3-[4-(4-piperidyl)anilino]piperidine-2,6-dione hydrochloride (Example 35, step 2) (200 mg, 0.618 mmol), tert-butyl 2-bromoacetate (CAS 5292-43-3) (157 mg, 0.119 ml, 0.803 mmol, 1.3 equiv.) and Hunig's base (399 mg, 0.539 ml, 3.09 mmol, 5 equiv.) in 4.0 ml of N,N-Dimethylformamide was stirred at room temperature for 2 hours. The reaction mixture was extracted with ethyl acetate and water. The aqueous layer was backextracted with ethyl acetate. The organic layers were combined, dried over sodium sulfate, filtered and concentrated to dryness. The crude product was purified by flash chromatography on a silica gel column eluting with an ethyl acetate:heptane 50:50 to 100:0 gradient. The desired tert-butyl 2-[4-[4-[[(3RS)-2,6-dioxo-3-piperidyl]amino]phenyl]-1-piperidyl]acetate (164 mg, 66% yield) was obtained as a white solid, MS: m/e=402.2 ([M+H]⁺).

### Step 4: 2-[4-[4-[(3RS)-2,6-Dioxo-3-piperidyl]amino]phenyl]-1-piperidyl]acetic acid hydrochloride

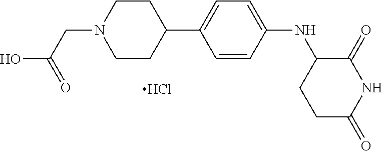 [SMILES: O=C(O)CN1CCC(c2ccc(NC3CCC(=O)NC3=O)cc2)CC1 ; Cl]

To a solution of tert-butyl 2-[4-[4-[[(3RS)-2,6-dioxo-3-piperidyl]amino]phenyl]-1-piperidyl]acetate (Example 35, step 3) (543 mg, 1.35 mmol) in 8.0 ml of ethyl acetate was added HCl (4 M in dioxane) (6.3 g, 6 ml, 24 mmol, 17.7 equiv.) at room temperature and stirring was continued for 72 hours. The product was collected by filtration, washed with ethyl acetate and dried under high vacuum. The desired 2-[4-[4-[(3RS)-2,6-dioxo-3-piperidyl]amino]phenyl]-1-piperidyl]acetic acid hydrochloride (quantitative yield) was obtained as light red solid, MS: m/e=346.2 ([M+H]⁺).

### Step 5: (2RS)-2-(6,7-Dihydro-5H-pyrrolo[1,2-c]imidazol-1-yl)-2-[7-fluoro-6-[6-[4-[2-[4-[4-[[(3RS)-2,6-dioxo-3-piperidyl]amino]phenyl]-1-piperidyl]acetyl]piperazin-1-yl]-3-pyridyl]indazol-2-yl]-N-thiazol-2-yl-acetamide

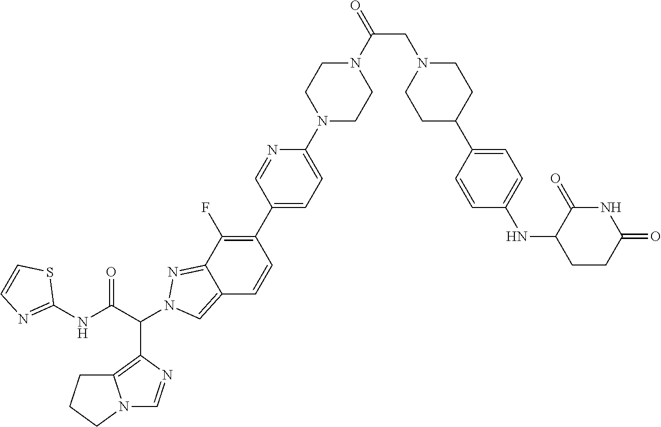 [SMILES: O=C1CCC(Nc2ccc(C3CCN(CC(=O)N4CCN(c5ccc(-c6ccc7cn(C(C(=O)Nc8nccs8)c8ncn9c8CCC9)nc7c6F)cn5)CC4)CC3)cc2)C(=O)N1]

The title compound, Compound 63, was obtained as a light grey solid, MS: m/e=871.7 ([M+H]⁺), using chemistry similar to that described in Example 1, step 14 starting from (2RS)-2-(6,7-dihydro-5H-pyrrolo[1,2-c]imidazol-1-yl)-2-[7-fluoro-6-(6-piperazin-1-yl-3-pyridyl)indazol-2-yl]-N-thiazol-2-yl-acetamide (Example 34, step 7) and 2-[4-[4-[(3RS)-2,6-dioxo-3-piperidyl]amino]phenyl]-1-piperidyl]acetic acid hydrochloride (Example 35, step 4).

### Example 36. Synthesis of 2-(6,7-dihydro-5H-pyrrolo[1,2-c]imidazol-1-yl)-2-[6-[6-[4-[2-[4-[4-[(2,6-dioxo-3-piperidyl)amino]-2-fluoro-phenyl]-1-piperidyl]acetyl]piperazin-1-yl]-3-pyridyl]-4-fluoro-indazol-2-yl]-N-thiazol-2-yl-acetamide, Compound 64

### Step 1: ethyl 2-(6-bromo-4-fluoro-indazol-2-yl)acetate

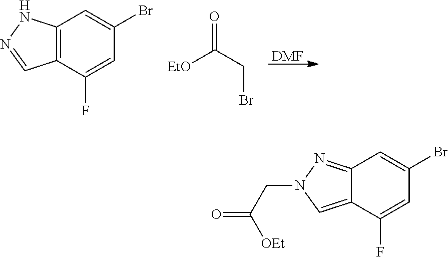

6-bromo-4-fluoro-1H-indazole (15.0 g, 69.76 mmol) and ethyl 2-bromoacetate (46.60 g, 279.04 mmol, 30.86 mL) in N,N-dimethylformamide (170 mL) were stirred at 100° C. for 35 h. The reaction mixture was cooled to ambient temperature and poured onto crushed ice. The mixture was extracted with ethyl acetate (300 mL×3). The combined organic layers were washed with 10% Sodium bicarbonate solution, brine, concentrated under reduced pressure and the residue was purified by silica gel chromatography (10-30% Ethyl acetate:Petroleum Ether) to afford ethyl 2-(6-bromo-4-fluoro-indazol-2-yl)acetate (11 g, 30.69 mmol, 44% yield) as an off white solid. LCMS (ESI+) m/z: 303.0 [M+H], ¹H-NMR (DMSO-d₆) δ 8.66 (s, 1H0, 7.77 (s, 1H), 7.08 (d, J=9.9 Hz, 1H), 5.44 (s, 2H), 4.18 (q, J=6.6 Hz, 2H), 1.22 (t, J=6.9 Hz, 3H).

### Step 2: tert-butyl 2-[2-(6-bromo-4-fluoro-indazol-2-yl)-3-ethoxy-3-oxo-propanoyl]pyrrolidine-1-carboxylate

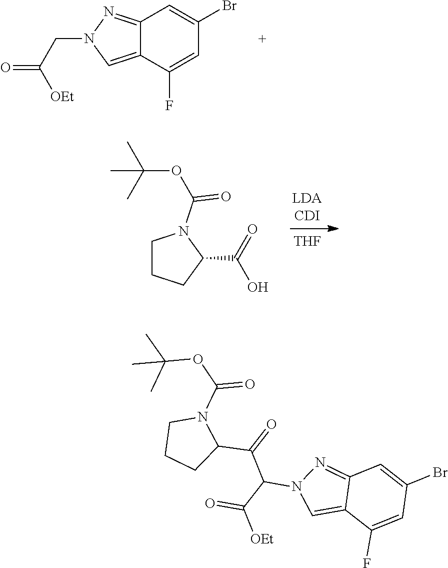 [SMILES: CCOC(=O)Cn1cc2c(F)cc(Br)cc2n1 ; CC(C)(C)OC(=O)N1CCC[C@H]1C(=O)O ; CCOC(=O)C(C(=O)C1CCCN1C(=O)OC(C)(C)C)n1cc2c(F)cc(Br)cc2n1 ; "THF"]

Ethyl 2-(6-bromo-4-fluoro-indazol-2-yl)acetate (10 g, 33.21 mmol) was dissolved in tetrahydrofuran (100 mL) and the solution was cooled to −78° C. Lithium diisopropylamide (0.7 M in tetrahydrofuran, 142 mL, 99.63 mmol) was added to the reaction mixture, upon which a yellow coloured precipitate was observed. The reaction mixture was stirred for 1 hour in −78° C. In a separate vessel, N-(tert-Butoxycarbonyl)-L-proline was dissolved in tetrahydrofuran (100 mL) and 1,1′-Carbonyldiimidazole (8.08 g, 49.82 mmol) was added under stirring. The reaction mixture was stirred for 1 hour. The N-(tert-Butoxycarbonyl)-L-proline/1,1′-Carbonyldiimidazole reaction mixture was slowly added to the 250 mL round bottom flask containing ethyl 2-(6-bromo-4-fluoro-indazol-2-yl)acetate and lithium diisopropylamide. The reaction mixture was stirred for 1 hour at −78° C., warmed to room temperature and stirred for 30 h at room temperature. A saturated ammonium chloride solution was added to the reaction mixture, and the organic layer was separated. Aqueous layer was extracted twice with ethyl acetate (250 mL×2). The combined organic layers were dried over sodium sulfate, concentrated under reduced pressure and the residue was purified by silica gel chromatography (0 to 100% ethyl acetate in petroleum ether) to afford tert-butyl 2-[2-(6-bromo-4-fluoro-indazol-2-yl)-3-ethoxy-3-oxo-propanoyl]pyrrolidine-1-carboxylate (8 g, 14.45 mmol, 44% yield) as a white solid. LCMS (ESI+): m/z 498.0/500.0 [M+H, Br pattern]

### Step 3: Ethyl 2-(6-bromo-4-fluoro-indazol-2-yl)-3-oxo-3-pyrrolidin-2-yl-propanoate hydrochloride

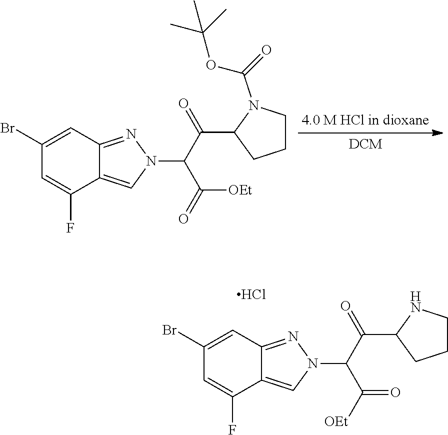 [SMILES: CCOC(=O)C(C(=O)C1CCCN1C(=O)OC(C)(C)C)n1cc2c(F)cc(Br)cc2n1 ; Cl ; CCOC(=O)C(C(=O)C1CCCN1)n1cc2c(F)cc(Br)cc2n1]

tert-Butyl 2-[2-(6-bromo-4-fluoro-indazol-2-yl)-3-ethoxy-3-oxo-propanoyl]pyrrolidine-1-carboxylate (16 g, 32.11 mmol) was dissolved in dichloromethane (160 mL) and the solution was cooled to 0° C. HCl (4.0 M in dioxane, 32.1 mL, 128.43 mmol) was added dropwise at 0° C. The reaction mixture was stirred at room temperature for 9 hours. The reaction mixture was evaporated to dryness to yield ethyl 2-(6-bromo-4-fluoro-indazol-2-yl)-3-oxo-3-pyrrolidin-2-yl-propanoate hydrochloride (13.5 g, 23.29 mmol, 72.55% yield) as a yellow colored solid. LCMS (ESI+): 398.0/400.1 (M+H, Br pattern).

### Step 4: Ethyl 2-(6-bromo-4-fluoro-indazol-2-yl)-2-(3-thioxo-2,5,6,7-tetrahydropyrrolo[1,2-c]imidazol-1-yl)acetate

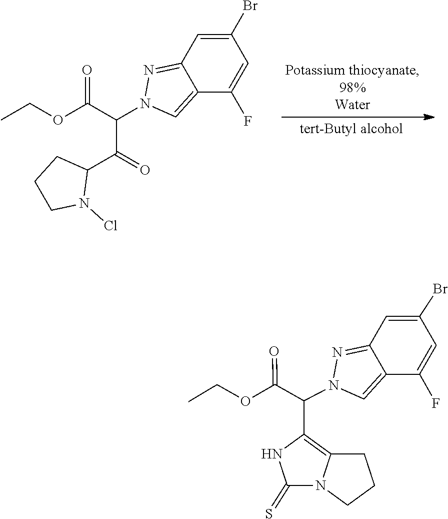 [SMILES: CCOC(=O)C(C(=O)C1CCCN1Cl)n1cc2c(F)cc(Br)cc2n1 ; CCOC(=O)C(c1[nH]c(=S)n2c1CCC2)n1cc2c(F)cc(Br)cc2n1]

Potassium thiocyanate (2.47 g, 25.43 mmol, 1.31 mL) was added to a stirred solution of ethyl 2-(6-bromo-4-fluoro-indazol-2-yl)-3-oxo-3-[pyrrolidin-2-yl]propanoate (6.75 g, 16.95 mmol) in Water (75 mL) and tert-butyl alcohol (24.5 mL) under a nitrogen atmosphere. The reaction mixture was heated to 90° C. for 8 hours. The reaction mixture was cooled to room temperature and extracted with 10% methanol in dichloromethane solution. The organic layer was dried over anhydrous sodium sulfate and concentrated. The residue was purified by silica gel chromatography (0 to 100% ethyl acetate in petroleum ether) to afford ethyl 2-(6-bromo-4-fluoro-indazol-2-yl)-2-(3-thioxo-2,5,6,7-tetrahydropyrrolo[1,2-c]imidazol-1-yl)acetate (2.9 g, 6.40 mmol, 38% yield) as a yellow colored solid. LCMS (ESI+) M/z: 439.0/441.0 [M+H, Br pattern], ¹H-NMR (400 MHz, DMSO-d₆) δ 12.11 (s, 1H), 8.69 (s, 1H), 7.81 (s, 1H), 7.11 (d, J=9.6 Hz, 1H), 6.65 (s, 1H), 4.31-4.19 (m, 2H), 3.81-3.69 (m, 2H), 2.62-2.80 (m, 2H), 2.49-2.39 (m, 2H), 1.20 (t, J=6.8 Hz, 3H).

### Step 5: ethyl 2-(6-bromo-4-fluoro-indazol-2-yl)-2-(6,7-dihydro-5H-pyrrolo[1,2-c]imidazol-1-yl)acetate

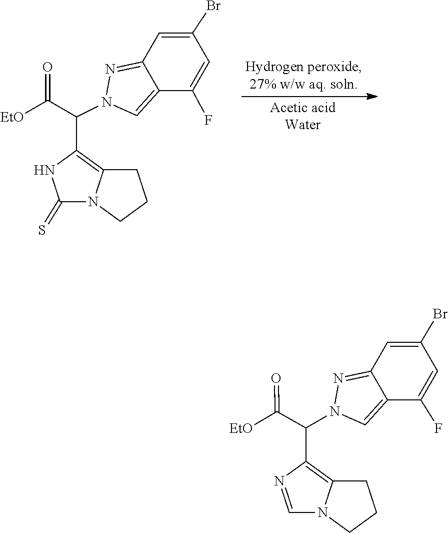 [SMILES: CCOC(=O)C(c1[nH]c(=S)n2c1CCC2)n1cc2c(F)cc(Br)cc2n1 ; CCOC(=O)C(c1ncn2c1CCC2)n1cc2c(F)cc(Br)cc2n1]

Ethyl 2-(6-bromo-4-fluoro-indazol-2-yl)-2-(3-thioxo-2,5,6,7-tetrahydropyrrolo[1,2-c]imidazol-1-yl)acetate (5.9 g, 13.43 mmol) was dissolved in Acetic acid (56 mL) and Water (19 mL). The solution was cooled to −10° C. Under a nitrogen atmosphere, Hydrogen peroxide (27% w/w aq. soln., stabilized, 1.37 g, 40.29 mmol, 1.25 mL) was added dropwise. The reaction was stirred at −10° C. for 100 minutes. A saturated sodium bicarbonate solution was added to the reaction mixture. The reaction mixture was extracted with ethyl acetate. The organic layer was washed with brine, dried over anhydrous sodium sulfate, filtered and concentrated to afford ethyl 2-(6-bromo-4-fluoro-indazol-2-yl)-2-(6,7-dihydro-5H-pyrrolo[1,2-c]imidazol-1-yl)acetate (4.1 g, 7.65 mmol, 69% yield) as a brown colored gum. LCMS (ESI+) M/z: 407 [M+H])

### Step 6: 2-[6-[6-(4-tert-butoxycarbonylpiperazin-1-yl)-3-pyridyl]-4-fluoro-indazol-2-yl]-2-(6,7-dihydro-5H-pyrrolo[1,2-c]imidazol-1-yl)acetic acid

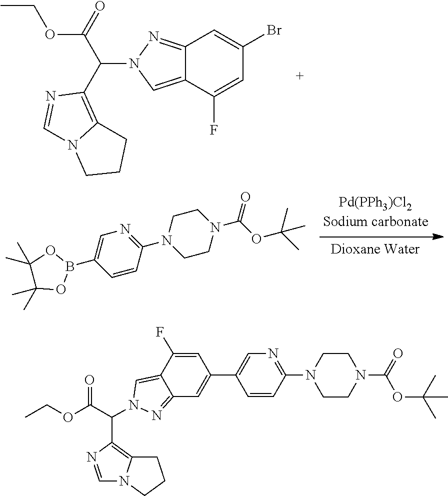

In a 100-mL sealed tube, ethyl 2-(6-bromo-4-fluoro-indazol-2-yl)-2-(6,7-dihydro-5H-pyrrolo[1,2-c]imidazol-1-yl)acetate (2.2 g, 5.40 mmol) and tert-butyl 4-[5-(4,4,5,5-tetramethyl-1,3,2-dioxaborolan-2-yl)-2-pyridyl]piperazine-1-carboxylate (2.52 g, 6.48 mmol) were dissolved in Dioxane (32 mL) and Water (8 mL). Sodium carbonate (1.15 g, 10.80 mmol, 452.64 μL) was added and the reaction mixture was purged with nitrogen for 5 min. Dichlorobis(triphenylphosphine)palladium(II) (379.29 mg, 540.23 μmol) was added under nitrogen atmosphere and the tube was sealed. The reaction was stirred at 90° C. in a heating block for 16 h. The reaction mixture was filtered over celite and washed with ethyl acetate. The organic layer was separated, washed with brine and concentrated. The residue was purified by silica gel chromatography (1% to 5% methanol in dichloromethane) to afford tert-butyl 4-[5-[2-[1-(6,7-dihydro-5H-pyrrolo[1,2-c]imidazol-1-yl)-2-ethoxy-2-oxo-ethyl]-4-fluoro-indazol-6-yl]-2-pyridyl]piperazine-1-carboxylate (1.1 g, 1.36 mmol, 25.2% yield) as an off-white solid. LCMS m/z: 590.0 [M+H]

### Step 7: 2-[6-[6-(4-tert-butoxycarbonylpiperazin-1-yl)-3-pyridyl]-4-fluoro-indazol-2-yl]-2-(6,7-dihydro-5H-pyrrolo[1,2-c]imidazol-1-yl)acetic acid

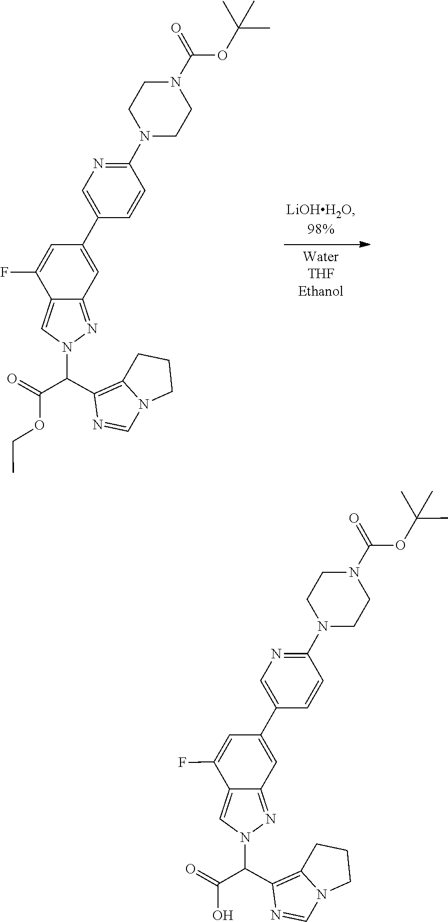 [SMILES: CCOC(=O)C(c1ncn2c1CCC2)n1cc2c(F)cc(-c3ccc(N4CCN(C(=O)OC(C)(C)C)CC4)nc3)cc2n1 ; CC(C)(C)OC(=O)N1CCN(c2ccc(-c3cc(F)c4cn(C(C(=O)O)c5ncn6c5CCC6)nc4c3)cn2)CC1]

tert-Butyl 4-[5-[2-[1-(6,7-dihydro-5H-pyrrolo[1,2-c]imidazol-1-yl)-2-ethoxy-2-oxo-ethyl]-4-fluoro-indazol-6-yl]-2-pyridyl]piperazine-1-carboxylate (1.1 g, 1.87 mmol) was dissolved in ethanol (8 mL) tetrahydrofuran (8 mL), and water (8 mL). Lithium hydroxide monohydrate, 98% (156.56 mg, 3.73 mmol) was added at ambient temperature and the reaction mixture was further stirred at ambient temperature for 5 h. The reaction mixture was adjusted to pH 5-6 with an aqueous potassium bisulfate solution and the mixture was extracted with 10% methanol-dichloromethane (100 ml×2). The organic layer was concentrated under reduced pressure. The resulting solid was stirred in ether, the ether layer was decanted and discarded, and the solid residue was dried under vacuum to afford compound 2-[6-[6-(4-tert-butoxycarbonylpiperazin-1-yl)-3-pyridyl]-4-fluoro-indazol-2-yl]-2-(6,7-dihydro-5H-pyrrolo[1,2-c]imidazol-1-yl)acetic acid (0.75 g, 1.07 mmol, 57% yield) as pale yellow solid. LCMS (ESI+): 561.9 (M+H).

### Step 8: tert-butyl 4-[5-[2-[1-(6,7-dihydro-5H-pyrrolo[1,2-c]imidazol-1-yl)-2-oxo-2-(thiazol-2-ylamino)ethyl]-4-fluoro-indazol-6-yl]-2-pyridyl]piperazine-1-carboxylate

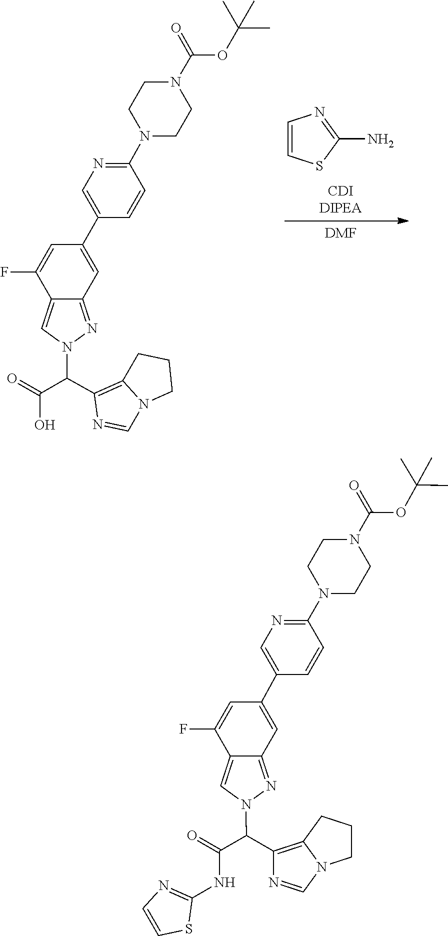

To a stirred solution of 2-[6-[6-(4-tert-butoxycarbonylpiperazin-1-yl)-3-pyridyl]-4-fluoro-indazol-2-yl]-2-(6,7-dihydro-5H-pyrrolo[1,2-c]imidazol-1-yl)acetic acid (0.2 g, 356.12 μmol) in N,N-dimethylformamide (7 mL) was added Carbonyldiimidazole (115.49 mg, 712.24 μmol) at RT and the mixture was stirred for 2 h. Thiazol-2-amine (46.36 mg, 462.96 μmol) was added and the reaction mixture was stirred at ambient temperature for 16 h. The reaction mixture was further stirred at 50° C. for 3 h. The reaction mixture was quenched with water and extracted 10% methanol in dichloromethane. The organic layer was concentrated under reduced pressure and the residue purified by silica gel chromatography (3% to 8% methanol in dichloromethane) to afford tert-butyl 4-[5-[2-[1-(6,7-dihydro-5H-pyrrolo[1,2-c]imidazol-1-yl)-2-oxo-2-(thiazol-2-yl amino)ethyl]-4-fluoro-indazol-6-yl]-2-pyridyl]piperazine-1-carboxylate (0.16 g, 245 μmol, 69% yield). LCMS (ESI+) m/z: 644.2 (M+H).

### Step 9: 2-(6,7-dihydro-5H-pyrrolo[1,2-c]imidazol-1-yl)-2-[4-fluoro-6-(6-piperazin-1-yl-3-pyridyl)indazol-2-yl]-N-thiazol-2-yl-acetamide

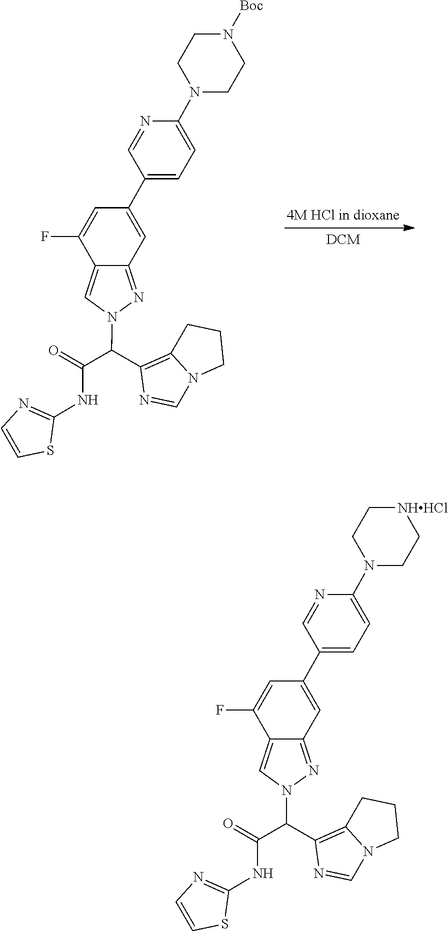

Hydrogen Chloride (4M in 1,4-dioxane, 0.5 mL, 2.0 mmol,) was added to a stirred solution of tert-butyl 4-[5-[2-[1-(6,7-dihydro-5H-pyrrolo[1,2-c]imidazol-1-yl)-2-oxo-2-(thiazol-2-ylamino)ethyl]-4-fluoro-indazol-6-yl]-2-pyridyl]piperazine-1-carboxylate (0.15 g, 233.02 μmol) in dichloromethane (8 mL) at 0° C. After addition the reaction mixture temperature was raised slowly to Room Temperature and stirred further for 5 h. The reaction mixture was concentrated under reduced pressure. Diethyl ether (15 mL) was added to the solid residue and the mixture was stirred for 15 min. The ether layer was decanted and discarded. The solid was dried under vacuum to afford 2-(6,7-dihydro-5H-pyrrolo[1,2-c]imidazol-1-yl)-2-[4-fluoro-6-(6-piperazin-1-yl-3-pyridyl)indazol-2-yl]-N-thiazol-2-yl-acetamide (0.13 g, 221.9 μmol, 95.2% yield) as a brown solid. LCMS (ESI+): m/z 544.2 (M+H).

### Step 10: 2-(6,7-dihydro-5H-pyrrolo[1,2-c]imidazol-1-yl)-2-[6-[6-[4-[2-[4-[4-[(2,6-dioxo-3-piperidyl)amino]-2-fluoro-phenyl]-1-piperidyl]acetyl]piperazin-1-yl]-3-pyridyl]-4-fluoro-indazol-2-yl]-N-thiazol-2-yl-acetamide

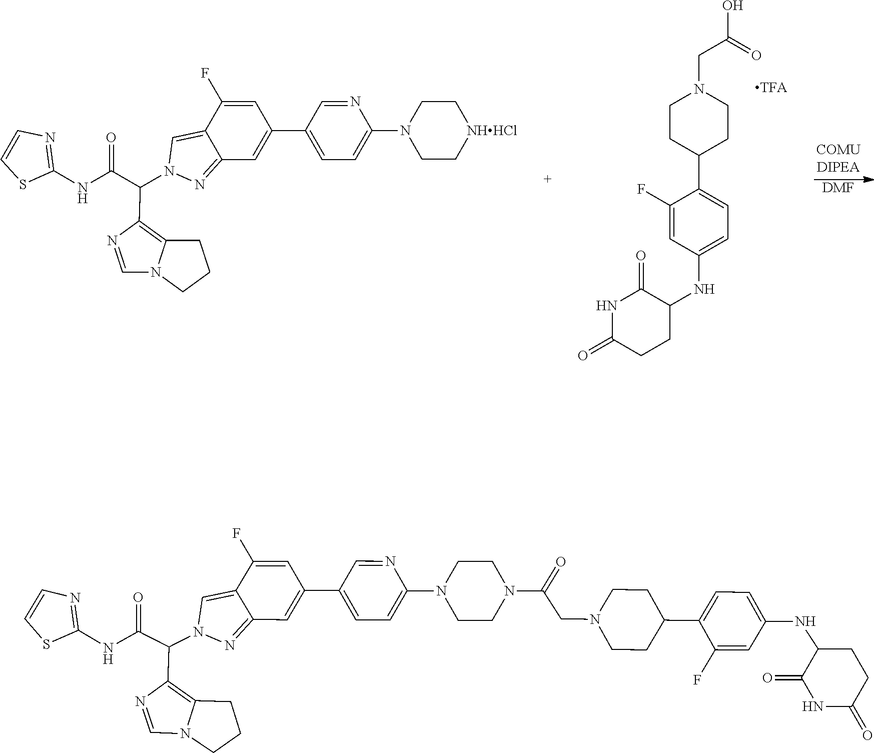

N,N-diisopropylethylamine (189.17 μL, 140.37 mg, 1.09 mmol) was added to a stirred solution of 2-(6,7-dihydro-5H-pyrrolo[1,2-c]imidazol-1-yl)-2-[4-fluoro-6-(6-piperazin-1-yl-3-pyridyl)indazol-2-yl]-N-thiazol-2-yl-acetamide hydrochloride (90 mg, 155.15 μmol) and 2-[4-[4-[(2,6-dioxo-3-piperidyl)amino]-2-fluoro-phenyl]-1-piperidyl]acetic acid hydrochloride (56.38 mg, 141.00 μmol) in N,N-dimethylformamide (1.5 mL) at 0° C. The mixture was stirred for 10 min. COMU was added (99.67 mg, 232.73 μmol), and reaction mixture was stirred for a further 2 h. The reaction mixture was concentrated and the residue was purified by reverse phase silica gel chromatography (C18, 0:100 to 100:0 Acetonitrile:0.1% Ammonium acetate in water). The desired fractions were pooled, frozen and lyophilized. The residue was further purified by

Preparative HPLC. (Purification method: Column: X-Bridge C₈ (50×4.6 mm), 3.5 μm; Mobile Phase A: 10 mM Ammonium acetate in milli-q water; Mobile phase B: Acetonitrile) to afford Compound 64 (26 mg, 29.04 μmol, 19% yield) as an off white solid. LCMS (m/z: 889.2, [M+H]), ¹H-NMR (400 MHz, DMSO-d₆) δ 13.01-12.66 (br. S, 1H), 10.79 (s, 1H), 8.57 (d, J=2.6 Hz, 1H), 8.28 (s, 1H), 7.99 (dd, J=8.9, 2.6 Hz, 1H), 7.69 (d, J=6.1 Hz, 2H), 7.50 (d, J=3.7 Hz, 1H), 7.39-7.09 (m, 2H), 6.99 (t, J=8.6 Hz, 2H), 6.68 (s, 1H), 6.61-6.26 (m, 2H), 6.00 (d, J=7.8 Hz, 1H), 4.30 (dt, J=11.8, 6.2 Hz, 1H), 4.14-3.90 (m, 2H), 3.68 (d, J=30.0 Hz, 5H), 3.58 (s, 4H), 3.22 (s, 2H), 2.94 (d, J=10.7 Hz, 2H), 2.89-2.65 (m, 1H), 2.58 (m, 4H), 2.09 (d, J=8.8 Hz, 2H), 1.91-1.80 (m, 1H), 1.66 (s, 5H).

### Example 37. Synthesis of 2-(6,7-dihydro-5H-pyrrolo[1,2-c]imidazol-1-yl)-2-[6-[6-[4-[2-[4-[4-[(2,6-dioxo-3-piperidyl)amino]phenyl]-1-piperidyl]acetyl]piperazin-1-yl]-3-pyridyl]-4-fluoro-indazol-2-yl]-N-thiazol-2-yl-acetamide, (Compound 65)

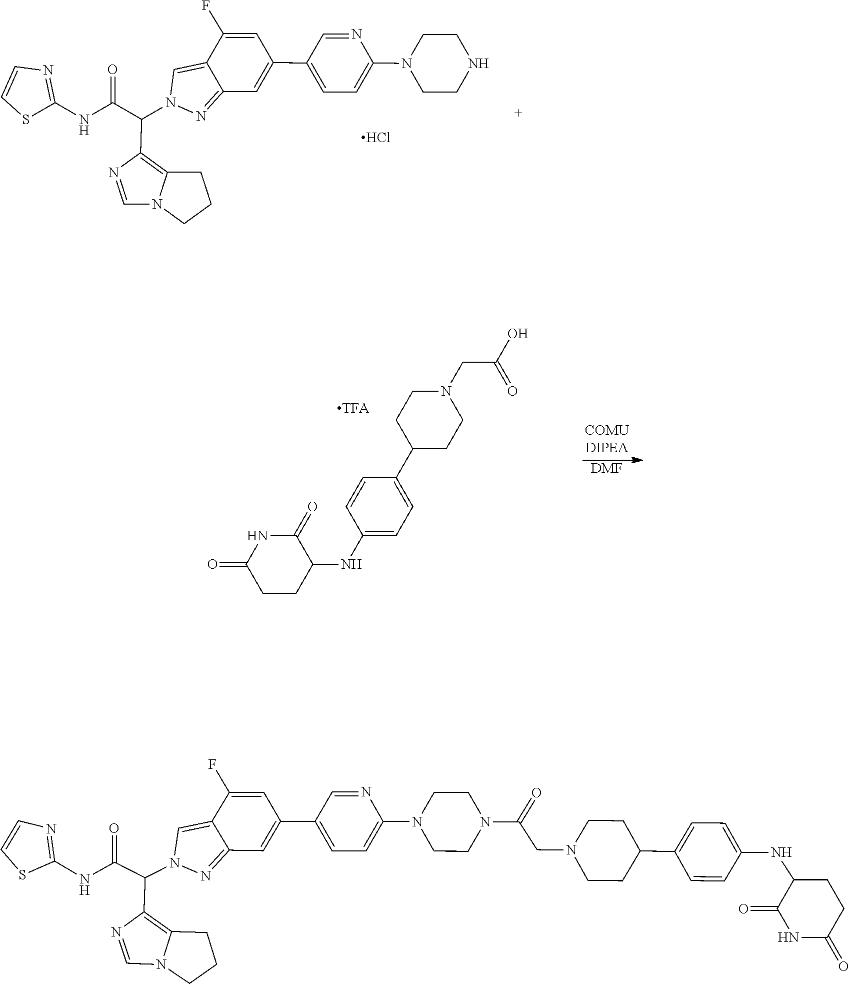

N,N-diisopropylethylamine (151.51 mg, 1.17 mmol, 204.19 μL) was added to a stirred solution of 2-(6,7-dihydro-5H-pyrrolo[1,2-c]imidazol-1-yl)-2-[4-fluoro-6-(6-piperazin-1-yl-3-pyridyl)indazol-2-yl]-N-thiazol-2-yl-acetamide hydrochloride (0.085 g, 146.53 μmol) and 2-[4-[4-[(2,6-dioxo-3-piperidyl)amino]phenyl]-1-piperidyl]acetic acid trifluoroacetic acid (50.61 mg, 110.16 μmol) in N,N-dimethylformamide (3 mL) at 0° C. The mixture was stirred for 15 min. COMU was added (94.12 mg, 219.80 μmol) and the temperature was slowly raised to RT and stirred for 3 h. The reaction mixture was concentrated and the residue was purified on a Reverse phase column (C18), eluting with a 10 to 50% acetonitrile (0.1% TFA) in water (0.1% TFA) gradient. The desired fractions were lyophilized to afford the a dry solid. A 10% sodium bicarbonate solution was added, and the aqueous layer was extracted with ethyl acetate (×2). The organic layers were combined, concentrated and lyophilized to afford Compound 65 (10 mg, 9.84 μmol, 6.7% yield) as an off white solid. LCMS m/z: 871.1 [M+H], ¹H NMR (400 MHz, DMSO-d₆) δ 12.83 (s, 1H), 10.78 (s, 1H), 8.58 (d, J=2.6 Hz, 1H), 8.29 (d, J=0.9 Hz, 1H), 8.00 (dd, J=8.9, 2.6 Hz, 1H), 7.70 (d, J=3.7 Hz, 2H), 7.52 (d, J=3.6 Hz, 1H), 7.29 (d, J=3.6 Hz, 1H), 7.20 (dd, J=12.2, 1.1 Hz, 1H), 6.98 (dd, J=11.7, 8.6 Hz, 3H), 6.71 (s, 1H), 6.61 (d, J=8.1 Hz, 2H), 5.67 (d, J=7.2 Hz, 1H), 4.27 (dt, J=11.9, 6.7 Hz, 1H), 4.13-3.91 (m, 2H), 3.62 (q, J=31.7, 26.3 Hz, 9H), 3.22 (s, 2H), 2.95 (s, 1H), 2.85-2.69 (m, 1H), 2.64-2.55 (m, 2H), 2.52 (s, 4H), 2.20-2.01 (m, 2H), 1.95-1.79 (m, 1H), 1.78-1.47 (m, 3H).

### Example 38. Synthesis of 2-(6,7-dihydro-5H-pyrrolo[1,2-c]imidazol-1-yl)-2-[6-[6-[4-[2-[4-[5-[(2,6-dioxo-3-piperidyl)amino]-2-pyridyl]-1-piperidyl]acetyl]piperazin-1-yl]-3-pyridyl]-4-fluoro-indazol-2-yl]-N-thiazol-2-yl-acetamide (Compound 66)

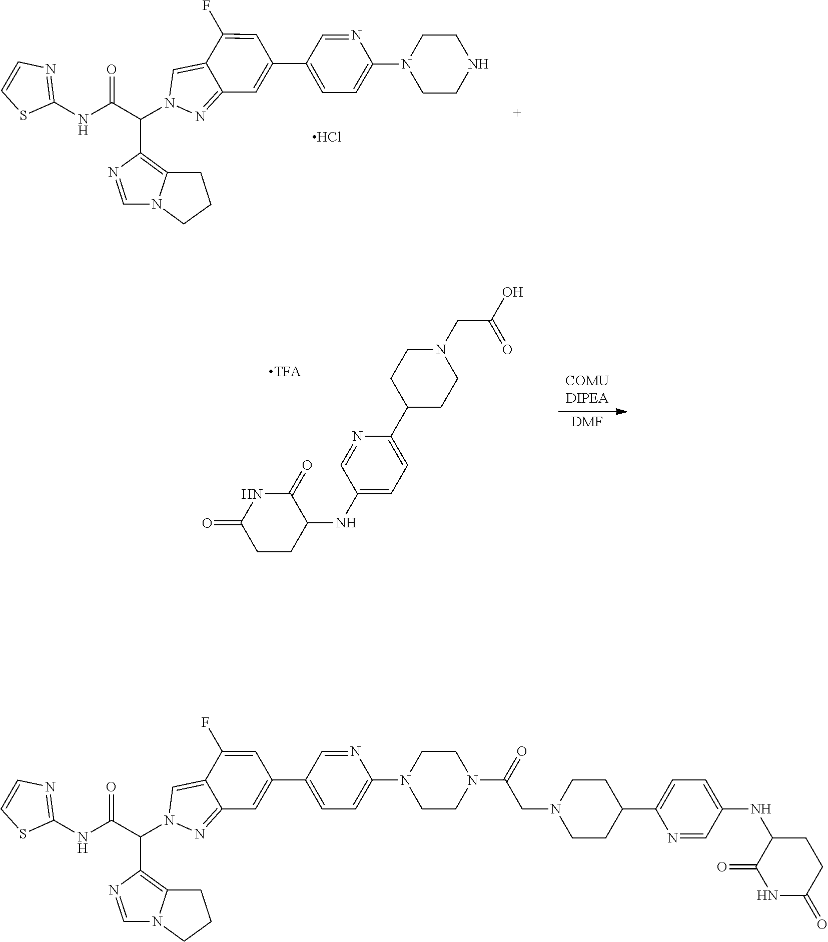

N,N-diisopropylethylamine (151.51 mg, 1.17 mmol, 204.19 μL) was added to a stirred solution of 2-(6,7-dihydro-5H-pyrrolo[1,2-c]imidazol-1-yl)-2-[4-fluoro-6-(6-piperazin-1-yl-3-pyridyl)indazol-2-yl]-N-thiazol-2-yl-acetamide hydrochloride (0.085 g, 146.53 μmol) and 2-[4-[5-[(2,6-dioxo-3-piperidyl)amino]-2-pyridyl]-1-piperidyl]acetic acid, trifluoroacetic acid salt (50.76 mg, 110.24 μmol) in N,N-dimethylformamide (3 mL) at 0° C. The reaction mixture was stirred for 15 min. COMU (94.12 mg, 219.80 μmol) was then added and the temperature was slowly raised to RT. The reaction mixture was stirred for 3 h. The reaction mixture was concentrated under reduced pressure. The crude residue was purified on reverse phase column (C18), eluting with a 10 to 50% acetonitrile (0.1% TFA) in water (0.1% TFA) gradient. The desired fractions were lyophilized. The residue was further purified by preparative HPLC (Purification method: Column: XBRIDGE C8 (4.6×50 mm), 3.5 μm; Mobile Phase A: 10 mM Ammonium acetate in water; Mobile phase B: Acetonitrile) to afford Compound 66 (15.5 mg, 16.85 μmol, 11.50% yield) as an off white solid. LCMS (ESI+): m/z 872.1 [M+H]; ¹H NMR (400 MHz, DMSO-d₆) δ 12.85 (br. s, 1H), 10.80 (s, 1H), 8.57 (d, J=2.6 Hz, 1H), 8.28 (s, 1H), 8.08-7.84 (m, 2H), 7.69 (d, J=4.8 Hz, 2H), 7.50 (d, J=3.6 Hz, 1H), 7.42-7.08 (m, 2H), 7.05-6.79 (m, 3H), 6.69 (s, 1H), 5.93 (d, J=7.8 Hz, 1H), 4.33 (dt, J=12.5, 6.0 Hz, 1H), 4.14-3.92 (m, 2H), 3.85-3.44 (m, 8H), 3.21 (s, 2H), 2.93 (d, J=10.7 Hz, 2H), 2.87-2.52 (m, 2H), 2.50-2.40 (m, 4H), 2.17-2.00 (m, 3H), 2.00-1.38 (m, 5H).

### Example 39. Synthesis of 2-(6,7-dihydro-5H-pyrrolo[1,2-c]imidazol-1-yl)-2-[6-[6-[4-[2-[4-[3-(2,4-dioxohexahydropyrimidin-1-yl)-1-methyl-indazol-6-yl]-1-piperidyl]acetyl]piperazin-1-yl]-3-pyridyl]-4-fluoro-indazol-2-yl]-N-thiazol-2-yl-acetamide (Compound 67)

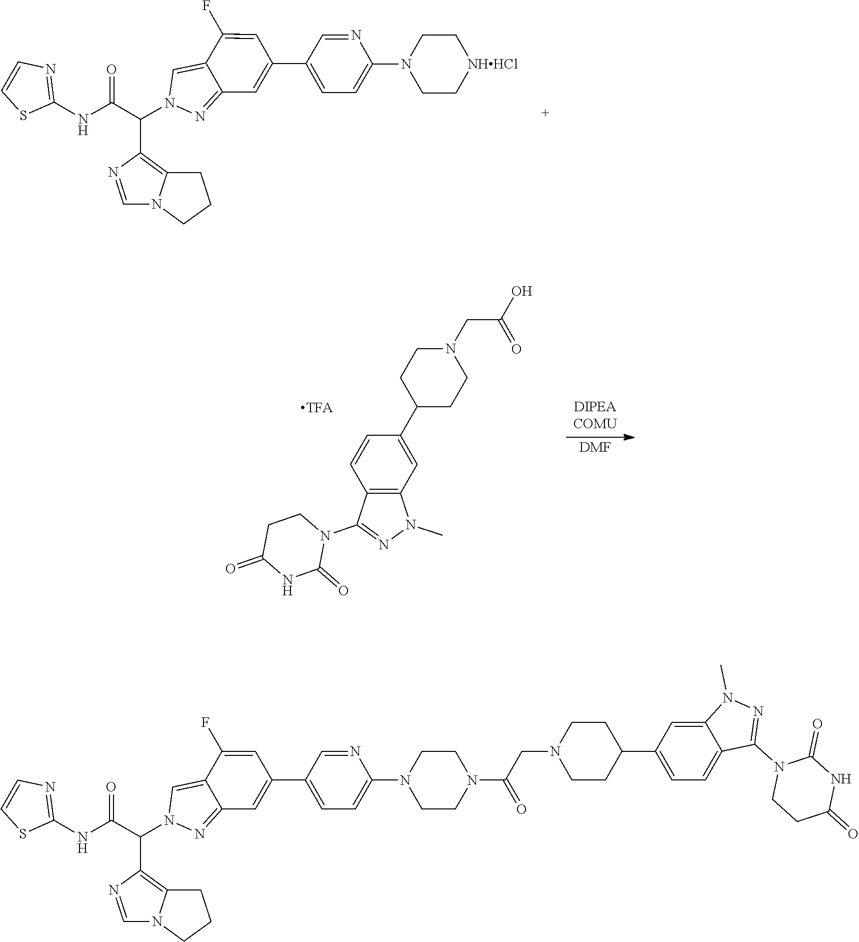

N,N-diisopropylethylamine (133.68 mg, 1.03 mmol, 180.16 μL) was added to a stirred solution of 2-(6,7-dihydro-5H-pyrrolo[1,2-c]imidazol-1-yl)-2-[4-fluoro-6-(6-piperazin-1-yl-3-pyridyl)indazol-2-yl]-N-thiazol-2-yl-acetamide hydrochloride (0.075 g, 129.29 μmol) and 2-[4-[3-(2,4-dioxohexahydropyrimidin-1-yl)-1-methyl-indazol-6-yl]-1-piperidyl]acetic acid, trifluoroacetic acid salt (71.03 mg, 142.22 μmol) in N,N-dimethylformamide (3 mL) at 0° C. The reaction mixture was stirred for 5 min. COMU (83.06 mg, 193.94 μmol) was added at 0° C. and the reaction was further stirred for 2 h. The reaction mixture was concentrated and the residue was purified by reverse phase silica gel chromatography (C18, 1:1 0.1% Ammonium acetate in water:Acetonitrile). The desired fractions were lyophilized to afford Compound 67 (38.4 mg, 41.11 μmol, 32% yield) as an off white solid. LCMS (ESI−): m/z 909.3 [M−H]. ¹H NMR (400 MHz, DMSO-d₆) δ 12.82 (s, 1H), 10.54 (s, 1H), 8.57 (d, J=2.6 Hz, 1H), 8.29 (d, J=0.9 Hz, 1H), 8.00 (dd, J=9.0, 2.6 Hz, 1H), 7.70 (d, J=3.8 Hz, 2H), 7.61-7.47 (m, 2H), 7.44 (s, 1H), 7.29 (d, J=3.5 Hz, 1H), 7.20 (dd, J=12.2, 1.1 Hz, 1H), 7.13-7.02 (m, 1H), 6.99 (d, J=8.9 Hz, 1H), 6.71 (s, 1H), 4.09-3.98 (m, 2H), 3.97 (s, 3H), 3.91 (t, J=6.6 Hz, 2H), 3.71 (d, J=29.0 Hz, 4H), 3.60 (s, 5H), 3.26 (s, 3H), 3.01 (d, J=10.6 Hz, 2H), 2.89-2.71 (m, 3H), 2.67-2.53 (m, 2H), 2.27-2.12 (m, 2H), 1.89-1.68 (m, 4H).

### Example 40. 2-(6,7-dihydro-5H-pyrrolo[1,2-c]imidazol-1-yl)-2-[6-[6-[2-[2-[4-[4-[[(3S)-2,6-dioxo-3-piperidyl]amino]-2-fluoro-phenyl]-1-piperidyl]acetyl]-2,6-diazaspiro[3.3]heptan-6-yl]-3-pyridyl]-7-fluoro-indazol-2-yl]-N-thiazol-2-yl-acetamide, (Compound 69)

### Step 1: Synthesis of tert-butyl 6-(5-(2-(1-(6,7-dihydro-5H-pyrrolo[1,2-c]imidazol-1-yl)-2-ethoxy-2-oxoethyl)-7-fluoro-2H-indazol-6-yl)pyridin-2-yl)-2,6-diazaspiro[3.3]heptane-2-carboxylate

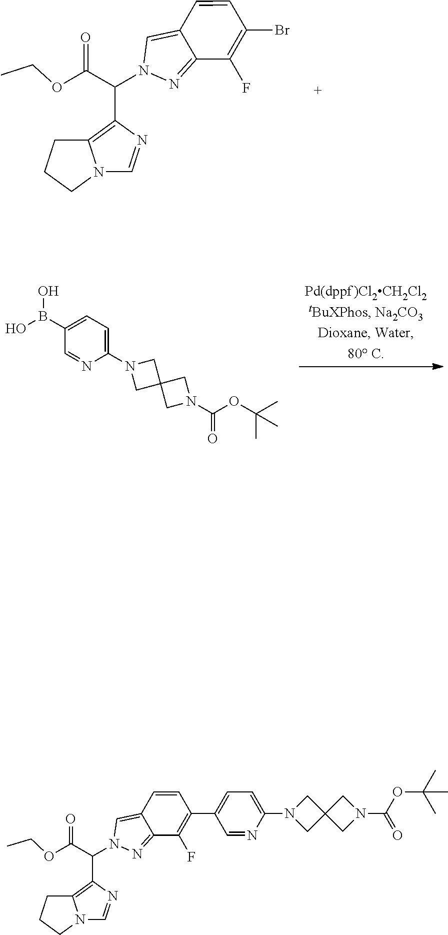 [SMILES: CCOC(=O)C(c1ncn2c1CCC2)n1cc2ccc(Br)c(F)c2n1 ; CC(C)(C)OC(=O)N1CC2(C1)CN(c1ccc(B(O)O)cn1)C2 ; CCOC(=O)C(c1ncn2c1CCC2)n1cc2ccc(-c3ccc(N4CC5(CN(C(=O)OC(C)(C)C)C5)C4)nc3)c(F)c2n1]

In a 50-mL sealed tube, ethyl 2-(6-bromo-7-fluoro-indazol-2-yl)-2-(6,7-dihydro-5H-pyrrolo[1,2-c]imidazol-1-yl)acetate (0.83 g, 2.04 mmol) and [6-(2-tert-butoxycarbonyl-2,6-diazaspiro[3.3]heptan-6-yl)-3-pyridyl]boronic acid (780.59 mg, 2.45 mmol) in 1,4-dioxane (8 mL) was added Sodium carbonate (540.05 mg, 5.10 mmol, 213.46 μL) in Water (2 ml). The reaction mixture was degassed with nitrogen for 10 minutes. [1,1′-Bis(diphenylphosphino)ferrocene]dichloropalladium(II), complex with dichloromethane (166.43 mg, 203.81 μmol) and 2-di-tert-butylphosphino-2′,4′,6′-triisopropylbiphenyl (86.55 mg, 203.81 μmol) was added under nitrogen atmosphere and the mixture was further degassed with nitrogen for 5 minutes. The tube was sealed and was stirred at 80° C. in a heating block for 5 h. The reaction mixture was filtered over celite and washed with ethyl acetate. was separated from the aqueous layer. The organic layer was dried over sodium sulfate, filtered, and concentrated under reduced pressure. The crude residue purified by flash column chromatography on silica gel (0-10% Methanol in Dichloromethane) to give tert-butyl 6-[5-[2-[1-(6,7-dihydro-5H-pyrrolo[1,2-c]imidazol-1-yl)-2-ethoxy-2-oxo-ethyl]-7-fluoro-indazol-6-yl]-2-pyridyl]-2,6-diazaspiro[3.3]-heptane-2-carboxylate (0.43 g, 630.35 μmol, 30.93% yield) as a brown solid. LCMS (ESI+) m/z: 602.3 [M+H]⁺

### Step 2: Synthesis of lithium 2-(6-(6-(6-(tert-butoxycarbonyl)-2,6-diazaspiro[3.3]heptan-2-yl)pyridin-3-yl)-7-fluoro-2H-indazol-2-yl)-2-(6,7-dihydro-5H-pyrrolo[1,2-c]imidazol-1-yl)acetate

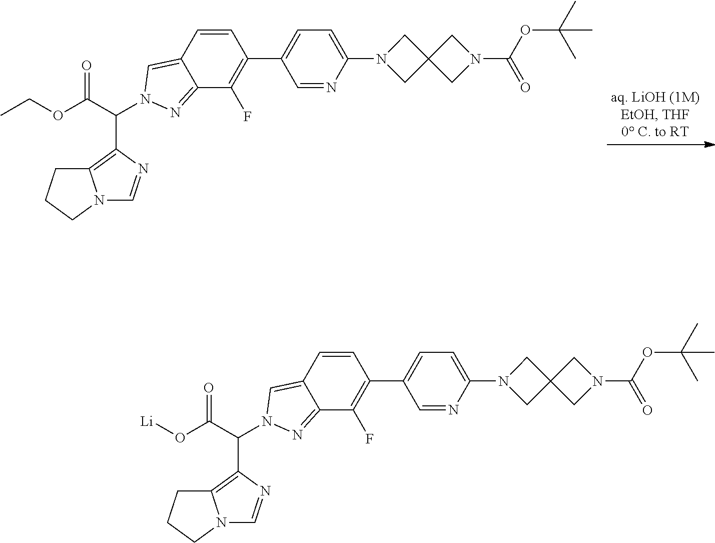

To a stirred solution of tert-butyl 6-[5-[2-[1-(6,7-dihydro-5H-pyrrolo[1,2-c]imidazol-1-yl)-2-ethoxy-2-oxo-ethyl]-7-fluoro-indazol-6-yl]-2-pyridyl]-2,6-diazaspiro[3.3]heptane-2-carboxylate (0.42 g, 698.06 μmol) in ethanol (3 mL) and tetrahydrofuran (3 mL) was added lithium hydroxide (1M aqueous, 0.9 mL, 907.47 μmol) at ambient temperature and the reaction mixture was stirred for 3 h. The mixture was concentrated under reduced pressure to afford solid, which was further triturated with diethyl ether, decanted and dried to get lithium 2-(6-(6-(6-(tert-butoxycarbonyl)-2,6-diazaspiro[3.3]heptan-2-yl)pyridin-3-yl)-7-fluoro-2H-indazol-2-yl)-2-(6,7-dihydro-5H-pyrrolo[1,2-c]imidazol-1-yl)acetate (0.38 g, 529.13 μmol, 93.9% yield) as a brown solid. LCMS (ESI+) m/z: 574.3 [M+H]⁺

### Step 3: tert-butyl 6-(5-(2-(1-(6,7-dihydro-5H-pyrrolo[1,2-c]imidazol-1-yl)-2-oxo-2-(thiazol-2-ylamino)ethyl)-7-fluoro-2H-indazol-6-yl)pyridin-2-yl)-2,6-diazaspiro[3.3]heptane-2-carboxylate

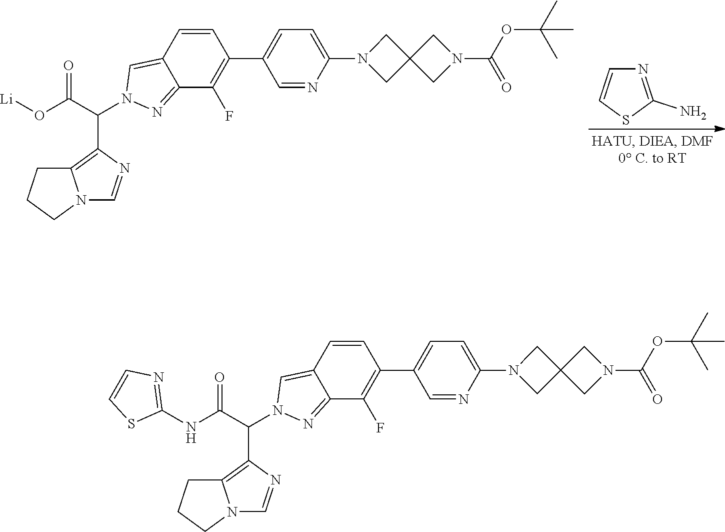

To a stirred solution of [2-[6-[6-(2-tert-butoxycarbonyl-2,6-diazaspiro[3.3]heptan-6-yl)-3-pyridyl]-7-fluoro-indazol-2-yl]-2-(6,7-dihydro-5H-pyrrolo[1,2-c]imidazol-1-yl)acetyl]oxylithium (0.37 g, 638.43 μmol) in N,N-dimethylformamide (8 mL) was added N,N-Diisopropylethylamine (495.07 mg, 3.83 mmol, 667.21 μL) at 0° C. 1-[Bis(dimethylamino)methylene]-1H-1,2,3-triazolo[4,5-b]pyridinium 3-oxid hexafluoro-phosphate (364.12 mg, 957.64 μmol) was added at the same temperature. Thiazol-2-amine (95.90 mg, 957.64 μmol) was added and the reaction mixture was stirred at room temperature for 16 h. The reaction mixture was added ice cold water and obtained solid was filtered, washed with water, and dried by an air stream. The crude solid residue was purified by flash chromatography using silica (0-8% Methanol in Dichloromethane) to afford tert-butyl 6-[5-[2-[1-(6,7-dihydro-5H-pyrrolo[1,2-c]imidazol-1-yl)-2-oxo-2-(thiazol-2-yl amino)ethyl]-7-fluoro-indazol-6-yl]-2-pyridyl]-2,6-diazaspiro[3.3]heptane-2-carboxylate (0.23 g, 323.74 μmol, 50.71% yield) as a brown solid. LCMS (ESI+) m/z: 656.3 [M+H]t

### Step 4: Synthesis of 2-(6-(6-(2,6-diazaspiro[3.3]heptan-2-yl)pyridin-3-yl)-7-fluoro-2H-indazol-2-yl)-2-(6,7-dihydro-5H-pyrrolo[1,2-c]imidazol-1-yl)-N-(thiazol-2-yl)acetamide, trifluoroacetic acid

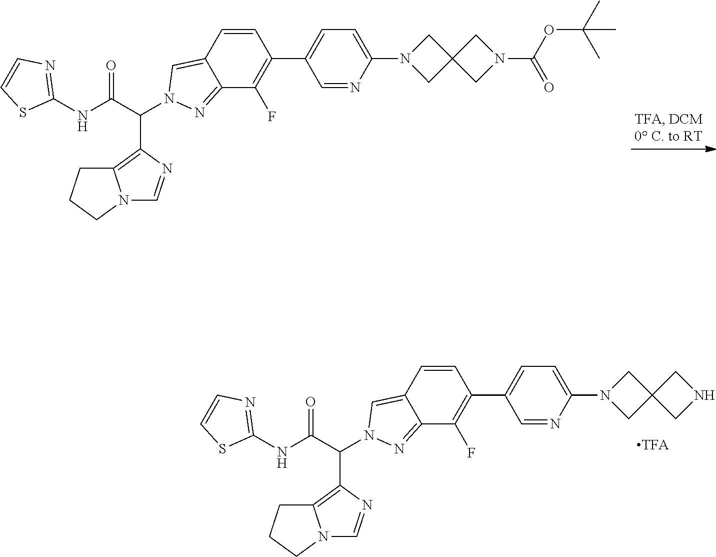 [SMILES: CC(C)(C)OC(=O)N1CC2(C1)CN(c1ccc(-c3ccc4cn(C(C(=O)Nc5nccs5)c5ncn6c5CCC6)nc4c3F)cn1)C2 ; O=C(Nc1nccs1)C(c1ncn2c1CCC2)n1cc2ccc(-c3ccc(N4CC5(CNC5)C4)nc3)c(F)c2n1]

To a stirred solution of tert-butyl 6-[5-[2-[1-(6,7-dihydro-5H-pyrrolo[1,2-c]imidazol-1-yl)-2-oxo-2-(thiazol-2-yl amino)ethyl]-7-fluoro-indazol-6-yl]-2-pyridyl]-2,6-diazaspiro[3.3]heptane-2-carboxylate (0.12 g, 183.00 μmol) in dichloromethane (10 mL) was added trifluoroacetic acid (83.46 mg, 731.99 μmol, 56.39 μL) dissolved in dichloromethane (2 mL) at 0° C. dropwise. The temperature of the reaction mixture was slowly raised to ambient temperature and stirred for 4 h. The reaction mixture was concentrated under reduced pressure, triturated at −40° C. with diethyl ether, decanted to afford 2-[6-[6-(2,6-diazaspiro[3.3]heptan-2-yl)-3-pyridyl]-7-fluoro-indazol-2-yl]-2-(6,7-dihydro-5H-pyrrolo[1,2-c]imidazol-1-yl)-N-thiazol-2-yl-acetamide, trifluoroacetic acid salt as a brown solid. LCMS (ESI+) m/z: 556.2 [M+H]⁺.

### Step 5: 2-(6,7-dihydro-5H-pyrrolo[1,2-c]imidazol-1-yl)-2-(6-(6-(6-(2-(4-(4-(((S)-2,6-dioxopiper-idin-3-yl)amino)-2-fluorophenyl)piperidin-1-yl)acetyl)-2,6-diazaspiro[3.3]heptan-2-yl)pyridin-3-yl)-7-fluoro-2H-indazol-2-yl)-N-(thiazol-2-yl)acetamide

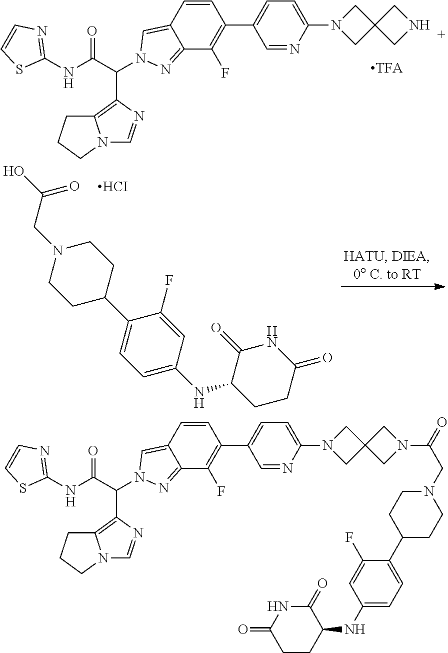

To a stirred solution of 2-[4-[4-[[(3S)-2,6-dioxo-3-piperidyl]amino]-2-fluoro-phenyl]-1-piperidyl]acetic acid; hydrochloride (48.84 mg, 122.14 μmol) in N,N-dimethylformamide (4 mL) at 0° C. was added N,N-Diisopropylethylamine (138.96 mg, 1.08 mmol, 187.28 μL). 1-[Bis(dimethylamino)methylene]-1H-1,2,3-triazolo[4,5-b]pyridinium 3-oxid hexafluorophosphate (76.65 mg, 201.60 μmol) was added at the same temperature. 2-[6-[6-(2,6-diazaspiro[3.3]heptan-2-yl)-3-pyridyl]-7-fluoro-indazol-2-yl]-2-(6,7-dihydro-5H-pyrrolo[1,2-c]imidazol-1-yl)-N-thiazol-2-yl-acetamide, trifluoroacetic acid salt (0.09 g, 134.40 μmol) was added and the reaction mixture was stirred for 2 h while warming to room temperature. The crude mixture was directly injected on a C18 column (50 g) for purification eluting (0% to 60% acetonitrile in water+0.1% ammonium acetate over 15 minutes, then steep gradient to 100% acetonitrile). The pure fractions were frozen and lyophilized to afford Compound 69 (60 mg, 64.82 μmol, 48.23% yield) as an off white solid. LCMS (ESI+) m/z: 899.3 [M−H]⁺. ¹H-NMR (400 MHz, DMSO-d₆: δ 12.81 (s, 1H), 10.79 (s, 1H), 8.33 (t, J=Hz, 2H), 7.79 (d, J=10.00 Hz, 1H), 7.69 (s, 1H), 7.59 (d, J=8.80 Hz, 1H), 7.52 (d, J=3.60 Hz, 1H), 7.28 (d, J=3.60 Hz, 1H), 7.12 (dd, J=8.60, 6.80 Hz, 1H), 7.02 (t, J=8.40 Hz, 1H), 6.72 (s, 1H), 6.53 (d, J=8.80 Hz, 1H), 6.48 (m, 2H), 6.01 (d, J=7.60 Hz, 1H), 4.45 (s, 2H), 4.31□4.30 (m, 1H), 4.16□4.14 (m, 4H), 4.09 (s, 2H), 4.02 (m, 2H), 3.00 (s, 2H), 2.92□2.89 (m, 2H), 2.68□2.84 (m, 2H), 2.52□2.60 (m, 4H), 2.07□2.10 (m, 3H), 1.88 (m, 1H), 1.69□1.65 (m, 4H).

### Example 41. 2-(6,7-dihydro-5H-pyrrolo[1,2-c]imidazol-1-yl)-2-[6-[6-[2-[2-[4-[3-(2,4-dioxohexahydropyrimidin-1-yl)-1-methyl-indazol-6-yl]-3,3-difluoro-1-piperidyl]acetyl]-2,6-diazaspiro[3.3]heptan-6-yl]-3-pyridyl]-7-fluoro-indazol-2-yl]-N-thiazol-2-yl-acetamide, (Compound 70)

### Step 1: Tert-butyl 3,3-difluoro-4-(trifluoromethylsulfonyloxy)-2,6-dihydropyridine-1-carboxylate

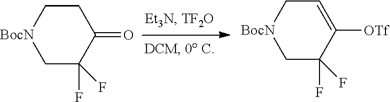 [SMILES: CC(C)(C)OC(=O)N1CCC(=O)C(F)(F)C1 ; CC(C)(C)OC(=O)N1CC=C(OS(=O)(=O)C(F)(F)F)C(F)(F)C1]

Triethylamine (3.23 g, 31.9 mmol, 4.44 mL) was added to a stirred solution of tert-butyl 3,3-difluoro-4-oxo-piperidine-1-carboxylate (2.5 g, 10.6 mmol) in dichloromethane (25 mL) at 0° C. Trifluoromethylsulfonic anhydride (4.50 g, 15.9 mmol, 2.68 mL) was added dropwise to the reaction mixture. The reaction was stirred at ambient temperature for 16 h. Then, the reaction was quenched with aqueous sodium bicarbonate, and extracted with dichloromethane, washed with brine, dried over sodium sulfate, and concentrated under reduced pressure. The residue was purified by silica gel chromatography (100% hexanes to 4:1 hexanes:ethyl acetate) to yield tert-butyl 3,3-difluoro-4-(trifluoromethylsulfonyloxy)-2,6-dihydropyridine-1-carboxylate (1.2 g, 2.29 mmol, 21% yield). ¹H NMR (400 MHz, Methanol-d4) δ 6.59 (s, 1H), 4.29 (q, J=4.3 Hz, 2H), 4.04 (t, J=11.0 Hz, 2H), 1.51 (s, 9H).

### Step 2: 1-[1-methyl-6-(4,4,5,5-tetramethyl-1,3,2-dioxaborolan-2-yl)indazol-3-yl]hexahydropyrimidine-2,4-dione

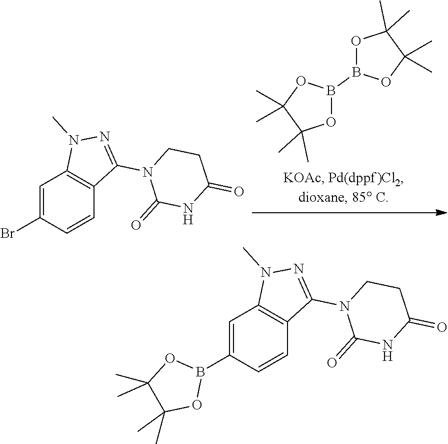

Potassium acetate (911 mg, 9.28 mmol) and Pd(dppf)Cl₂ (113 mg, 155 μmol) were added to a solution of 1-(6-bromo-1-methyl-indazol-3-yl)hexahydropyrimidine-2,4-dione (1.0 g, 3.09 mmol) and bis(pinacolato)diboron (1.18 g, 4.64 mmol) in 1,4-dioxane (15 mL). The mixture was stirred at 85° C. under a nitrogen atmosphere for 16 h. The mixture was cooled to ambient temperature and filtered through a pad of silica gel. The filter cake was washed with ethyl acetate and the filtrate was concentrated under reduced pressure. The residue was purified by silica gel chromatography (100% hexanes to 100% ethyl acetate) to yield 1-[1-methyl-6-(4,4,5,5-tetramethyl-1,3,2-dioxaborolan-2-yl)indazol-3-yl]hexahydropyrimidine-2,4-dione (1.1 g, 2.97 mmol, 96% yield). LCMS (ESI+): 371 (M+H).

### Step 3: tert-Butyl 4-(3-(2,4-dioxotetrahydropyrimidin-1(2H)-yl)-1-methyl-1H-indazol-6-yl)-3,3-difluoro-3,6-dihydropyridine-1(2H)-carboxylate

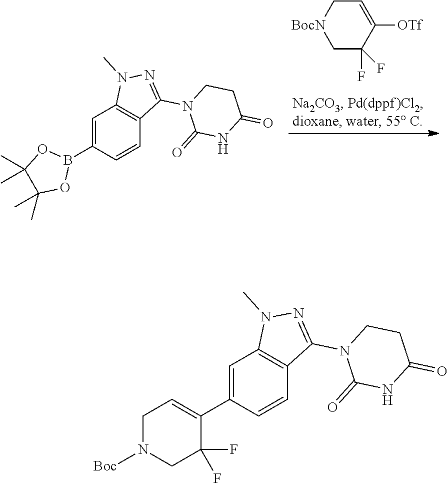 [SMILES: Cn1nc(N2CCC(=O)NC2=O)c2ccc(B3OC(C)(C)C(C)(C)O3)cc21 ; CC(C)(C)OC(=O)N1CC=C(OS(=O)(=O)C(F)(F)F)C(F)(F)C1 ; Cn1nc(N2CCC(=O)NC2=O)c2ccc(C3=CCN(C(=O)OC(C)(C)C)CC3(F)F)cc21]

Sodium carbonate (485 mg, 4.57 mmol) was added to a solution of 1-[1-methyl-6-(4,4,5,5-tetramethyl-1,3,2-dioxaborolan-2-yl)indazol-3-yl]hexahydropyrimidine-2,4-dione (677 mg, 1.83 mmol) and tert-butyl 3,3-difluoro-4-(trifluoromethylsulfonyloxy)-2,6-dihydropyridine-1-carboxylate (560 mg, 1.52 mmol) in 1,4-dioxane (10 mL) and water (2.5 mL) and the solvent was sparged with N₂ gas for 10 minutes. 1,1′-Bis(Diphenylphosphino)ferrocenepalladium (II) dichloride (111 mg, 152 μmol) was added and the reaction mixture was stirred at 55° C. for 2 h. The reaction mixture was cooled and diluted with water/ethyl acetate. After extraction, organic layer was washed with brine, dried over sodium sulfate, and concentrated. The residue was purified by silica gel chromatography (100% hexanes to 100% ethyl acetate) to give tert-butyl 4-[3-(2,4-dioxohexahydropyrimidin-1-yl)-1-methyl-indazol-6-yl]-3,3-difluoro-2,6-dihydropyridine-1-carboxylate (480 mg, 1.04 mmol, 68% yield). LCMS (ESI+): 462.2 (M+H)

### Step 4: tert-butyl 4-[3-(2,4-dioxohexahydropyrimidin-1-yl)-1-methyl-indazol-6-yl]-3,3-difluoro-piperidine-1-carboxylate

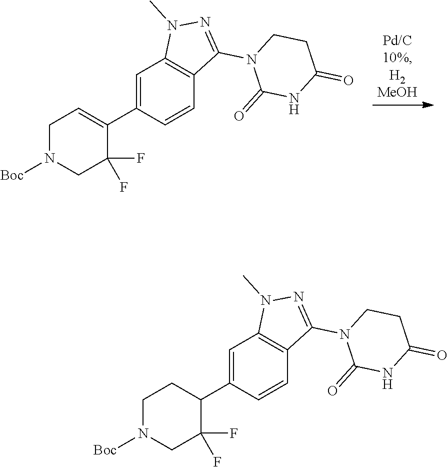 [SMILES: Cn1nc(N2CCC(=O)NC2=O)c2ccc(C3=CCN(C(=O)OC(C)(C)C)CC3(F)F)cc21 ; Cn1nc(N2CCC(=O)NC2=O)c2ccc(C3CCN(C(=O)OC(C)(C)C)CC3(F)F)cc21]

Palladium, 10% on carbon (Type 487, dry) (331 mg, 311 μmol) was added to a solution of tert-butyl 4-[3-(2,4-dioxohexahydropyrimidin-1-yl)-1-methyl-indazol-6-yl]-3,3-difluoro-2,6-dihydropyridine-1-carboxylate (478 mg, 1.04 mmol) in methanol (10.3 mL) and the mixture was stirred at ambient temperature under a hydrogen balloon atmosphere for 24 h. The hydrogen balloon was removed, and the mixture was diluted with dichloromethane (20 mL) and the slurry was stirred for additional 24 h. Then, the mixture was filtered through a pad of celite, washed using a solution of dichloromethane/methanol (3:1), and concentrated to afford tert-butyl 4-[3-(2,4-dioxohexahydropyrimidin-1-yl)-1-methyl-indazol-6-yl]-3,3-difluoro-piperidine-1-carboxylate (450 mg, 94% yield). LCMS (ESI+): 408.2 (M-tert-butyl+H).

### Step 5: 4-[3-(2,4-dioxohexahydropyrimidin-1-yl)-1-methyl-indazol-6-yl]-3,3-difluoro-piperidine hydrochloride

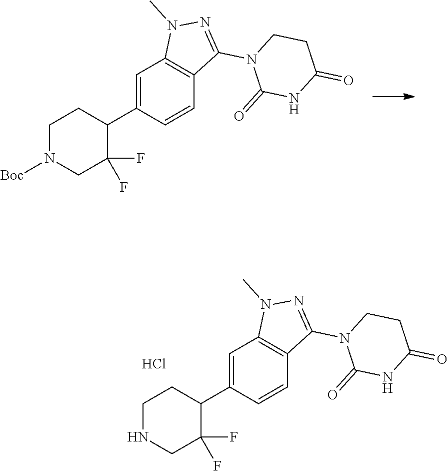 [SMILES: Cn1nc(N2CCC(=O)NC2=O)c2ccc(C3CCN(C(=O)OC(C)(C)C)CC3(F)F)cc21 ; Cn1nc(N2CCC(=O)NC2=O)c2ccc(C3CCNCC3(F)F)cc21 ; Cl]

4-[3-(2,4-dioxohexahydropyrimidin-1-yl)-1-methyl-indazol-6-yl]-3,3-difluoro-piperidine hydrochloride was obtained in quantitative yield from tert-butyl 4-[3-(2,4-dioxohexahydropyrimidin-1-yl)-1-methyl-indazol-6-yl]-3,3-difluoro-piperidine-1-carboxylate using General method B for the removal of the tert-butoxycarbonyl group. LCMS (ESI+): 354.2 (M+H)

### Step 6: tert-butyl 2-(4-(3-(2,4-dioxotetrahydropyrimidin-1(2H)-yl)-1-methyl-1H-indazol-6-yl)-3,3-difluoropiperidin-1-yl)acetate

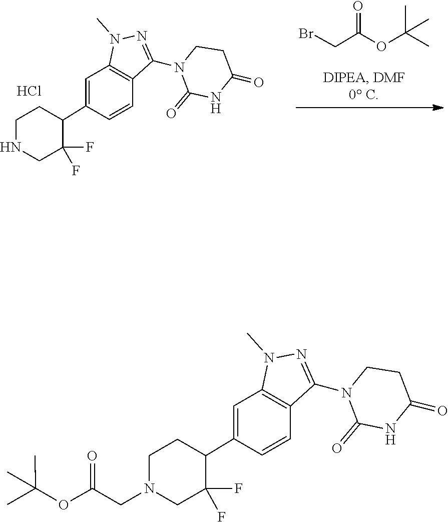

1-(6-(3,3-difluoropiperidin-4-yl)-1-methyl-1H-indazol-3-yl)dihydropyrimidine-2,4(1H,3H)-dione hydrochloride (1.75 g, 4.38 mmol) was dissolved in N,N-dimethylformamide (15 mL) and N,N-diisopropylethylamine (3.43 mL, 2.55 g, 19.7 mmol) was added. The mixture was cooled to 0° C., and tert-butyl 2-bromoacetate (770 μL, 1.02 g, 5.25 mmol) was added. The mixture was stirred at 0° C. for 4 h. The reaction was diluted with ethyl acetate and washed with saturated sodium bicarbonate and brine. The organic layer was concentrated and purified by silica gel chromatography (0-10% Methanol in dichloromethane) to yield tert-butyl 2-(4-(3-(2,4-dioxotetrahydropyrimidin-1(2H)-yl)-1-methyl-1H-indazol-6-yl)-3,3-difluoropiperidin-1-yl)acetate (1.90 g, 3.97 mmol, 90.6%) as a white solid. LCMS (ESI+): 478.3 (M+H)+

### Step 7: 2-(4-(3-(2,4-dioxotetrahydropyrimidin-1(2H)-yl)-1-methyl-1H-indazol-6-yl)-3,3-difluoropiperidin-1-yl)acetic acid, trifluoroacetic acid salt

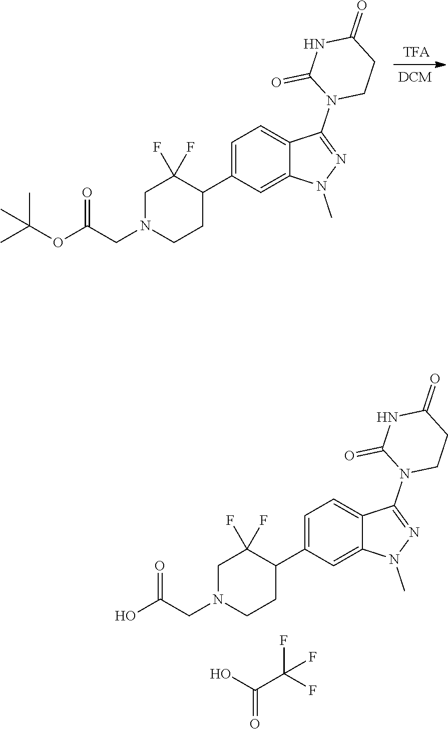

To the stirred solution of tert-butyl 2-[4-[3-(2,4-dioxohexahydropyrimidin-1-yl)-1-methyl-indazol-6-yl]-3,3-difluoro-1-piperidyl]acetate (100 mg, 209.42 μmol) in dichloromethane (5 mL) was added trifluoroacetic acid (1.48 g, 1.0 mL, 12.98 mmol) dropwise at 0° C. The reaction mixture stirred at ambient temperature for 3 h. The reaction mixture was concentrated under reduced pressure to give 2-[4-[3-(2,4-dioxohexahydropyrimidin-1-yl)-1-methyl-indazol-6-yl]-3,3-difluoro-1-piperidyl]acetic acid, trifluoroacetic acid salt (90 mg, 155.00 μmol, 74.01% yield) as an off-white solid. LCMS (ESI+): 422.2 (M+H)⁺

### Step 8: 2-(6,7-dihydro-5H-pyrrolo[1,2-c]imidazol-1-yl)-2-[6-[6-[2-[2-[4-[3-(2,4-dioxohexahydropyrimidin-1-yl)-1-methyl-indazol-6-yl]-3,3-difluoro-1-piperidyl]acetyl]-2,6-diazaspiro[3.3]heptan-6-yl]-3-pyridyl]-7-fluoro-indazol-2-yl]-N-thiazol-2-yl-acetamide

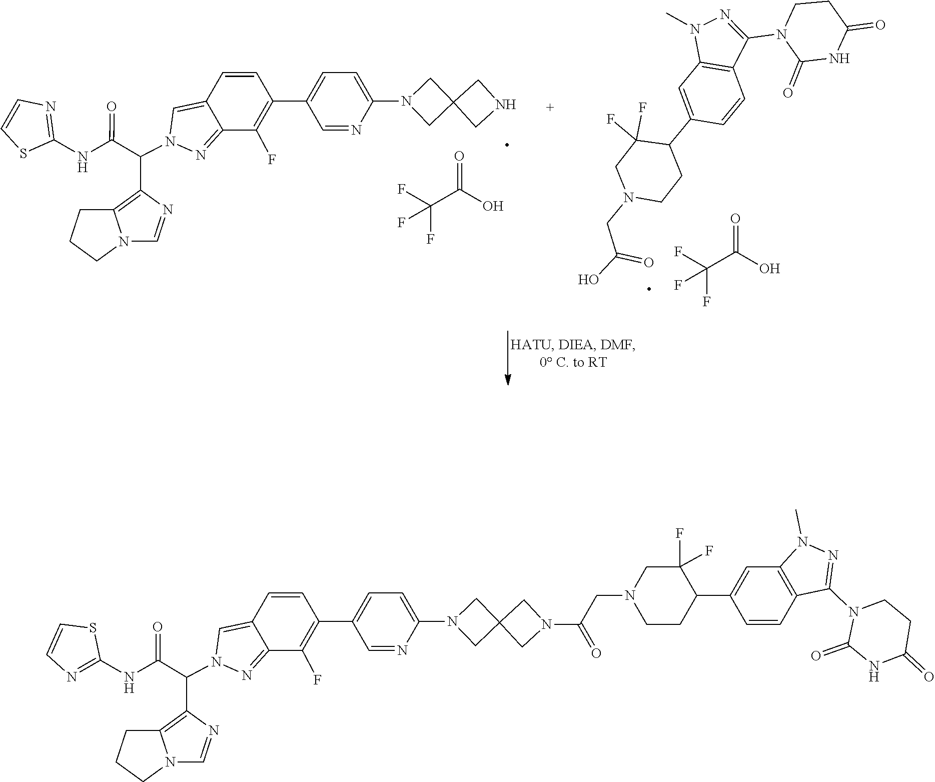

To a stirred solution of 2-[4-[3-(2,4-dioxohexahydropyrimidin-1-yl)-1-methyl-indazol-6-yl]-3,3-difluoro-1-piperidyl]acetic acid, trifluoroacetic acid salt (48.45 mg, 90.50 μmol) in N,N-dimethylformamide (5 mL) at 0° C. was added N,N-Diisopropylethylamine (108.08 mg, 836.26 μmol, 145.66 μL). 1-[Bis(dimethylamino)methylene]-1H-1,2,3-triazolo[4,5-b]pyridinium 3-oxid hexafluorophosphate (39.75 mg, 104.53 μmol) was added at the same temperature. 2-[6-[6-(2,6-diazaspiro[3.3]heptan-2-yl)-3-pyridyl]-7-fluoro-indazol-2-yl]-2-(6,7-dihydro-5H-pyrrolo[1,2-c]imidazol-1-yl)-N-thiazol-2-yl-acetamide, trifluoroacetic acid salt (70 mg, 104.53 μmol) was added and the reaction mixture was stirred for 2 h while warming to room temperature. The crude mixture was directly injected on a C18 column (50 g) for purification while eluting (0% to 60% of acetonitrile in water+0.1% ammonium acetate over 30 minutes, then steep gradient to 100% acetonitrile). The pure fractions were frozen and lyophilized to afford Compound 70 (41 mg, 42.08 μmol, 40.25% yield) as an off white solid. LCMS (ESI+) m/z: 959.3 [M+H]t 1H-NMR (400 MHz, DMSO-d₆: δ 12.85 (s, 1H), 10.58 (s, 1H), 8.34 (dd, J=9.60, 2.80 Hz, 2H), 7.80 (d, J=9.60 Hz, 1H), 7.69 (s, 1H), 7.61 (s, 1H), 7.58 (d, J=6.00 Hz, 2H), 7.52□7.51 (m, 1H), 7.29 (bs, 1H), 7.14□7.09 (m, 2H), 6.72 (bs, 1H), 6.55 (d, J=8.80 Hz, 1H), 4.45 (s, 2H), 4.16□4.12 (m, 6H), 4.04□4.02 (m, 2H), 4.00 (s, 3H), 3.93 (t, J=6.80 Hz, 2H), 3.26□3.23 (m, 4H), 3.01□2.98 (m, 1H), 2.84□2.83 (m, 1H), 2.76 (t, J□□□□□□ Hz, 2H), 2.52□2.51 (m, 2H), 2.50□2.40 (m, 2H), 2.33□2.30 (m, 1H), 1.89□1.81 (m, 1H) (A proton signal could not be observed due to water obscuration).

### Example 42. 2-(6,7-dihydro-5H-pyrrolo[1,2-c]imidazol-1-yl)-2-[6-[6-[2-[2-[1-[4-[(2,6-dioxo-3-piperidyl)amino]-3-fluoro-phenyl]-4-hydroxy-4-piperidyl]acetyl]-2,6-diazaspiro[3.3]heptan-6-yl]-3-pyridyl]-7-fluoro-indazol-2-yl]-N-thiazol-2-yl-acetamide, (Compound 71)

### Step 1: 1-(3-fluoro-4-nitrophenyl)piperidin-4-one

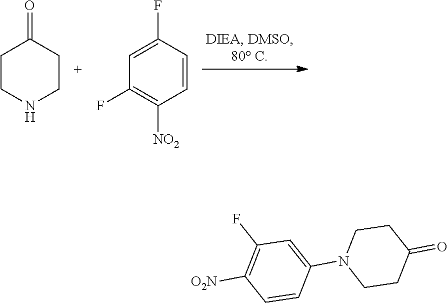

To a stirred solution of piperidin-4-one (13 g, 131.14 mmol), 2,4-difluoro-1-nitro-benzene (20.86 g, 131.14 mmol, 14.39 mL) in N,N-dimethylformamide (80 mL) was added N, N-Diisopropylethylamine (67.80 g, 524.56 mmol, 91.37 mL). The reaction mixture was stirred at 110° C. in a heating block for 16 h. The reaction mixture was diluted with ethyl acetate (500 mL), washed with cold water (150 mL). The organic layer was washed with brine solution (150 mL), dried over sodium sulphate and concentrated under reduced pressure to get crude. The residue was purified by column chromatography on silica gel eluted with 40% ethyl acetate in pet ether to afford 1-(3-fluoro-4-nitrophenyl) piperidin-4-one (9.0 g, 36.65 mmol, 27.95% yield) as brown solid. LCMS (ESI+) m/z: 239.1 [M+H]⁺.

### Step 2: tert-butyl 2-[1-(3-fluoro-4-nitrophenyl)-4-hydroxy-4-piperidyl]acetate

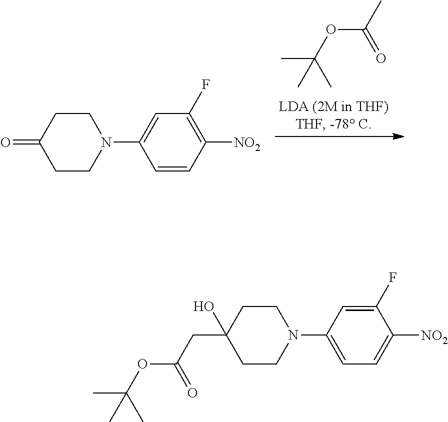

A round bottomed flask was charged with tert-butyl acetate (4.39 g, 37.78 mmol, 5.09 mL) in tetrahydrofuran (150 mL) and the solution was cooled to −78° C. Lithium diisopropylamide (2M solution in tetrahydrofuran, 75.56 mmol, 38 mL) was added dropwise over 15 minutes. The solution was stirred for 1 h at −78° C. 1-(3-fluoro-4-nitro-phenyl) piperidin-4-one (9.00 g, 37.78 mmol) in tetrahydrofuran (50 ml) was added to the reaction mixture at −78° C. and stirred at same reaction temperature for 2 h. The reaction mixture was slowly warmed to −40° C. The reaction mixture was quenched with ammonium chloride solution and extracted with ethyl acetate (600 mL). Organic layers were washed with brine (150 mL), dried over anhydrous sodium sulfate and concentrated under reduced pressure to get crude product. The crude residue was purified using silica gel column chromatography, eluting with 0-50% ethyl acetate in petroleum ether, to afford tert-butyl 2-[1-(3-fluoro-4-nitrophenyl)-4-hydroxy-4-piperidyl]acetate (11.91 g, 29.56 mmol, 77.28% yield) as brown solid. LCMS (ESI+) m/z: 355.1 [M+H]⁺.

### Step 3: tert-butyl 2-[1-(4-amino-3-fluoro-phenyl)-4-hydroxy-4-piperidyl]acetate

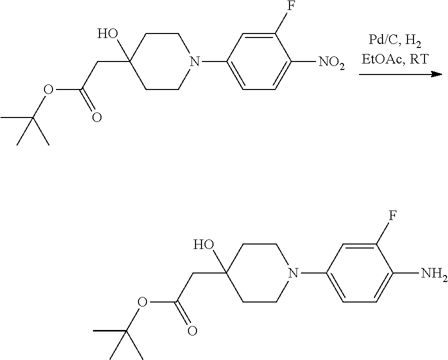 [SMILES: CC(C)(C)OC(=O)CC1(O)CCN(c2ccc([N+](=O)[O-])c(F)c2)CC1 ; CC(C)(C)OC(=O)CC1(O)CCN(c2ccc(N)c(F)c2)CC1]

A round bottomed flask was charged with tert-butyl 2-[1-(3-fluoro-4-nitro-phenyl)-4-hydroxy-4-piperidyl]acetate (11.91 g, 33.62 mmol) in water (4 mL), ethanol (20 mL) were added Fe powder (9.39 g, 168.11 mmol, 1.19 mL), ammonium chloride (5.40 g, 100.87 mmol, 3.53 mL) and stirred at 70° C. for 4 h. After completion of the reaction, the reaction mixture was filtered through celite and washed with ethyl acetate (200 mL). The filtrate was washed with water (80 mL), sodium bicarbonate solution (60 mL) and brine (60 mL). The organic layer was dried over sodium sulphate and concentrated under reduced pressure to get crude. The residue was purified by column chromatography on silica gel eluted with 70% ethyl acetate in pet ether to afford tert-butyl 2-[1-(4-amino-3-fluoro-phenyl)-4-hydroxy-4-piperidyl]acetate (8.5 g, 24.63 mmol, 73.26% yield) as brownish solid.. LCMS (ESI+) m/z: 325.2 [M+H]⁺.

### Step 4: tert-butyl 2-[1-[4-[(2,6-dioxo-3-piperidyl)amino]-3-fluoro-phenyl]-4-hydroxy-4-piperidyl]acetate

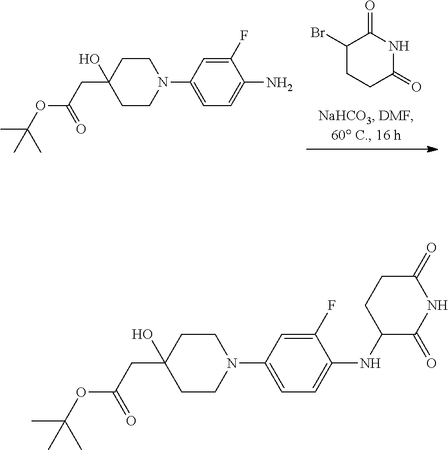 [SMILES: CC(C)(C)OC(=O)CC1(O)CCN(c2ccc(N)c(F)c2)CC1 ; O=C1CCC(Br)C(=O)N1 ; CC(C)(C)OC(=O)CC1(O)CCN(c2ccc(NC3CCC(=O)NC3=O)c(F)c2)CC1]

In a sealed tube, solution of tert-butyl 2-[1-(4-amino-3-fluoro-phenyl)-4-hydroxy-4-piperidyl]acetate (4.50 g, 13.87 mmol) in N,N-dimethylformamide (50 mL) was added Sodium bicarbonate (4.08 g, 48.55 mmol, 1.89 mL) and 3-bromopiperidine-2,6-dione (6.66 g, 34.68 mmol). The reaction tube was sealed and heated in a heating block at 70° C. for 16 h. Reaction mixture was cooled to room temperature, quenched with ice cooled water, extracted using ethyl acetate (200 ml) and washed with brine solution (50 ml). Organic layers were collected and concentrated under reduced pressure to afford crude residue. The crude product was purified using flash silica gel chromatography eluting with 0 to 70% ethyl acetate in petroleum ether to afford tert-butyl 2-[1-[4-[(2,6-dioxo-3-piperidyl)amino]-3-fluoro-phenyl]-4-hydroxy-4-piperidyl]acetate (5 g, 10.22 mmol, 73.66% yield) as a green solid. LCMS (ESI+) m/z: 436.3 [M+H]⁺

### Step 5: 2-(1-(4-((2,6-dioxopiperidin-3-yl) amino)-3-fluorophenyl)-4 hydroxypiperidin-4-yl) acetic acid; hydrochloride

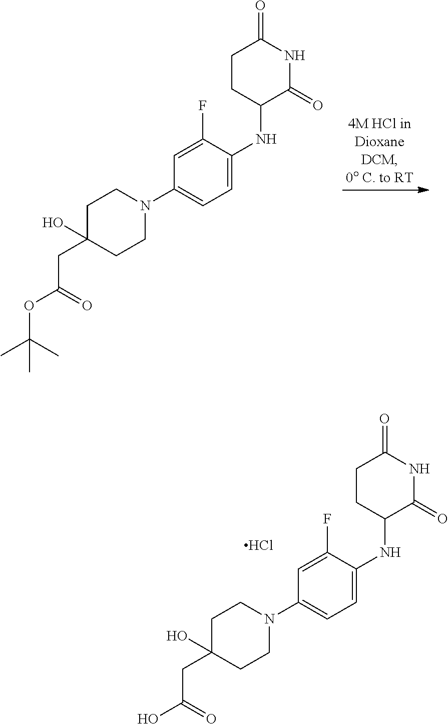 [SMILES: CC(C)(C)OC(=O)CC1(O)CCN(c2ccc(NC3CCC(=O)NC3=O)c(F)c2)CC1 ; O=C(O)CC1(O)CCN(c2ccc(NC3CCC(=O)NC3=O)c(F)c2)CC1 ; Cl]

To a stirred solution of tert-butyl 2-[1-[4-[(2,6-dioxo-3-piperidyl)amino]-3-fluoro-phenyl]-4-hydroxy-4-piperidyl]acetate (300 mg, 688.88 μmol) in dichloromethane (10 mL) at 0° C. under nitrogen added hydrogen chloride (4M in 1,4-dioxane, 1.38 mL, 201.15 mg, 5.51 mmol). The reaction mixture was concentrated under reduced pressure to afford solid residue. Solid residue was stirred in diethyl ether for 10 minutes, decanted and dried to afford 2-(1-(4-((2,6-dioxopiperidin-3-yl) amino)-3-fluorophenyl)-4 hydroxypiperidin-4-yl) acetic acid; hydrochloride (250.0 mg, 581.95 μmol, 84.48% yield). LCMS (ESI+) m/z: 380.1 [M+H]⁺

### Step 6: 2-(6,7-dihydro-5H-pyrrolo[1,2-c]imidazol-1-yl)-2-[6-[6-[2-[2-[1-[4-[(2,6-dioxo-3-piperidyl)amino]-3-fluoro-phenyl]-4-hydroxy-4-piperidyl]acetyl]-2,6-diazaspiro[3.3]heptan-6-yl]-3-pyridyl]-7-fluoro-indazol-2-yl]-N-thiazol-2-yl-acetamide

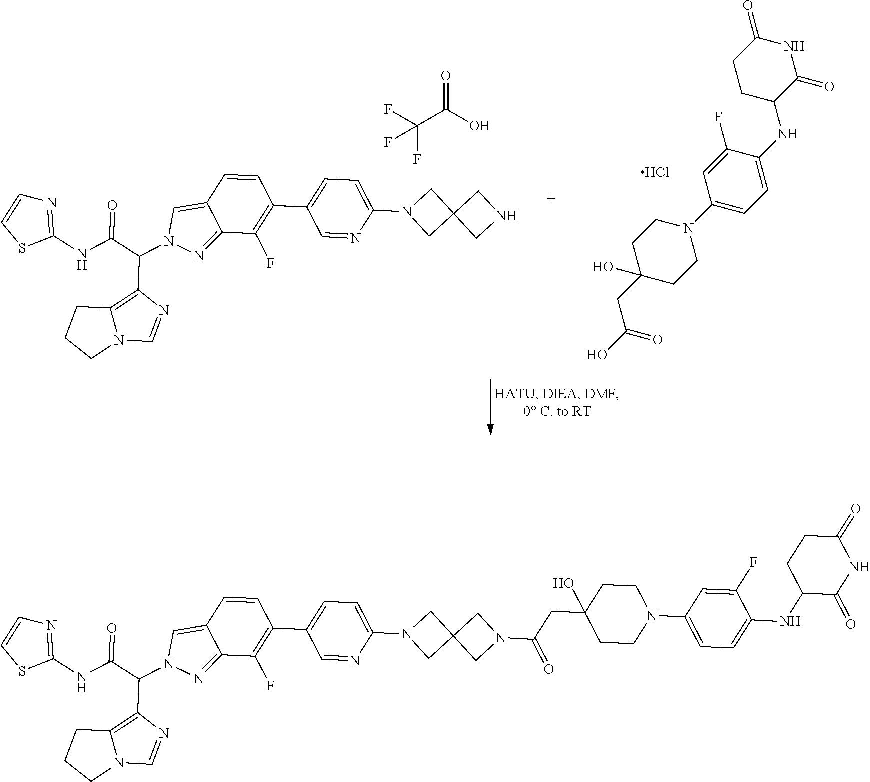

To a stirred solution of 2-[1-[4-[(2,6-dioxo-3-piperidyl)amino]-3-fluoro-phenyl]-4-hydroxy-4-piperidyl]acetic acid hydrochloride (43.47 mg, 104.53 μmol) in N,N-dimethylformamide (4 mL) at 0° C. was added N,N-diisopropylethylamine (108.08 mg, 836.26 μmol, 145.66 μL). 1-[Bis(dimethylamino)methylene]-1H-1,2,3-triazolo[4,5-b]pyridinium 3-oxide hexafluoro-phosphate (59.62 mg, 156.80 μmol) was added at the same temperature. 2-[6-[6-(2,6-diazaspiro[3.3]heptan-2-yl)-3-pyridyl]-7-fluoro-indazol-2-yl]-2-(6,7-dihydro-5H-pyrrolo[1,2-c]imidazol-1-yl)-N-thiazol-2-yl-acetamide, trifluoroacetic acid salt (0.07 g, 104.53 μmol) was added and the reaction mixture was stirred for 2 h while warming to room temperature. The crude mixture was directly injected on a C18 column (50 g) for purification, eluting with 0% to 60% acetonitrile in water (+0.1% ammonium acetate) over 30 minutes, then steep gradient to 100% acetonitrile. The pure fractions were frozen and lyophilized to afford product Compound 71 (45 mg, 48.19 μmol, 46.10% yield) as an off white solid. LCMS (ESI+) m/z: 917.3 [M+H]⁺; 1H-NMR (400 MHz, DMSO-d₆: δ 12.80 (s, 1H), 10.81 (s, 1H), 8.33□8.32 (m, 2H), 7.79 (d, J=9.20 Hz, 2H), 7.69 (s, 1H), 7.59 (d, J=8.80 Hz, 1H), 7.52 (d, J=3.60 Hz, 1H), 7.29 (d, J=3.60 Hz, 1H), 7.12 (dd, J=1.60, Hz, 1H), 6.74-6.73 (m, 3H), 6.59 (d, J=2.00 Hz, 1H), 6.54 (d, J=8.80 Hz, 1H), 5.04 (d, J=6.00 Hz, 1H), 4.78 (s, 1H), 4.38 (s, 2H), 4.31-4.26 (m, 1H), 4.15 (s, 4H), 4.09 (s, 2H), 4.04□4.02 (m, 2H), 3.18□03.15 (m, 2H), 2.95□2.93 (m, 2H), 2.84□2.76 (m, 2H), 2.52□2.51 (m, 2H), 2.22 (s, 2H), 2.09□2.08 (m, 1H), 1.98-1.97 (m, 1H), 1.75□1.73 (m, 2H), 1.64□1.61 (m, 2H) (water obscuration).

### Example 43. Step 1: 2-(6,7-dihydro-5H-pyrrolo[1,2-c]imidazol-1-yl)-2-[6-[6-[2-[2-[1-[4-[(2,6-dioxo-3-piperidyl)amino]-2-fluoro-phenyl]-4-hydroxy-4-piperidyl]acetyl]-2,6-diazaspiro[3.3]heptan-6-yl]-3-pyridyl]-7-fluoro-indazol-2-yl]-N-thiazol-2-yl-acetamide

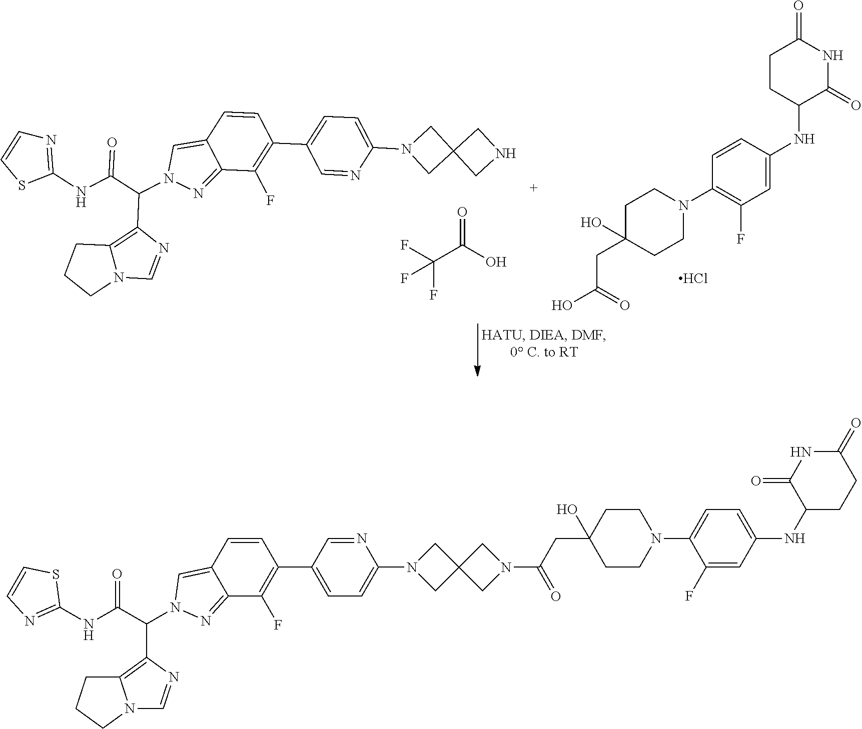

To a mixture of 2-[6-[6-(2,6-diazaspiro[3.3]heptan-2-yl)-3-pyridyl]-7-fluoro-indazol-2-yl]-2-(6,7-dihydro-5H-pyrrolo[1,2-c]imidazol-1-yl)-N-thiazol-2-yl-acetamide, trifluoroacetic acid salt (100 mg, 149.33 μmol) and 2-[1-[4-[(2,6-dioxo-3-piperidyl)amino]-2-fluoro-phenyl]-4-hydroxy-4-piperidyl]acetic acid hydrochloride (56.65 mg, 136.24 μmol) in N,N-dimethylformamide (2 mL) were added N,N-Diisopropylethylamine (57.90 mg, 447.99 μmol, 78.03 μL) at 0° C. 1-[Bis(dimethylamino)methylene]-1H-1,2,3-triazolo[4,5-b]pyridinium 3-oxid hexafluoro-phosphate (85.17 mg, 224.00 μmol) was added and the reaction mixture was stirred for 2 h at room temperature. The reaction mixture was directly injected on C18 column (50 g) for purification while eluting with (0% to 55% acetonitrile in water (+0.1% ammonium acetate) over 30 minutes, then steep gradient to 100% acetonitrile). The pure fractions were frozen and lyophilized to get Compound 74 (37.01 mg, 37.12 μmol, 24.86% yield) as grey colour solid. LCMS (ESI+) m/z: 915.3 [M−H]⁺. 1H-NMR (400 MHz, DMSO-d₆): δ 12.81 (s, 1H), 10.79 (s, 1H), 8.33 (d, J=4.80 Hz, 2H), 7.80 (d, J=4.80 Hz, 1H), 7.69 (s, 1H), 7.59 (d, J=8.80 Hz, 1H), 7.52 (d, J=3.60 Hz, 1H), 7.30 (s, 1H), 7.12 (d, J=1.60 Hz, 1H), 6.52□6.41 (m, 3H), 5.78 (d, J=7.6 Hz, 1H), 4.77 (s, 1H), 4.40 (s, 2H), 4.29-04.20 (m, 1H), 4.09 (bs, 4H), 4.02 (d, J=7.6 Hz, 2H), 4.00 (bs, 2H), 2.90□2.56 (m, 12H), 2.33 (s, 2H), 2.12-2.01 (m, 1H), 1.90-1.71 (m, 3H), 1.69-1.56 (m, 1H) (Water obscuration).

### Example 44. 2-(6,7-dihydro-5H-pyrrolo[1,2-c]imidazol-1-yl)-2-[6-[6-[2-[2-[1-[4-[[(3S)-2,6-dioxo-3-piperidyl]amino]-2-fluoro-phenyl]-4-hydroxy-4-piperidyl]acetyl]-2,6-diazaspiro[3.3]heptan-6-yl]-3-pyridyl]-4-fluoro-indazol-2-yl]-N-thiazol-2-yl-acetamide, (Compound 75)

### Step 1: tert-butyl 2-[1-[4-[[(3S)-2,6-dioxo-3-piperidyl]amino]-2-fluoro-phenyl]-4-hydroxy-4-piperidyl]acetate

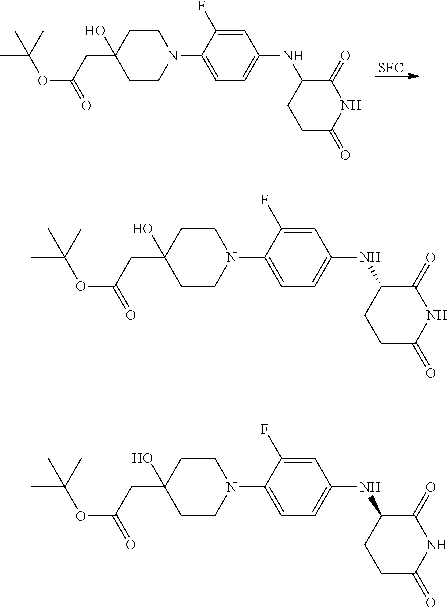

The racemic mixture tert-butyl 2-[1-[4-[(2,6-dioxo-3-piperidyl)amino]-2-fluoro-phenyl]-4-hydroxy-4-piperidyl]acetate (2 g, 4.59 mmol) was resolved by chiral SFC. 2.0 g of tert-butyl 2-[1-[4-[(2,6-dioxo-3-piperidyl)amino]-2-fluoro-phenyl]-4-hydroxy-4-piperidyl]acetate was dissolved in 22.0 ml of acetonitrile. SFC separation conditions: Column: LUX A1 [250×10 mm, 5-micron particle size]; Mobile phase: CO₂: Isopropanol (45:55); Flow rate: 12 g/min; Cycle time: 11.0 minute; Back pressure: 100 bar UV collection, wavelength: 254 nm; Volume: 0.4 ml per injection The first eluting set of fractions was evaporated under pressure to afford tert-butyl 2-[1-[4-[[(3S)-2,6-dioxo-3-piperidyl]amino]-2-fluoro-phenyl]-4-hydroxy-4-piperidyl]acetate (850 mg, 1.84 mmol, 40.04% yield) as an off white solid. LCMS m/z: 436.0 [M+H], LCMS (ESI+) m/z: 436.2 [M+H]+. 1H-NMR (400 MHz, DMSO-d₆): δ 10.77 (s, 1H), 6.83 (t, J=12.00 Hz, 1H), 6.49 (d, J=20.00 Hz, 1H), 6.41 (d, J=12.00 Hz, 1H), 5.77 (d, J=7.60 Hz, 1H), 4.44 (s, 1H), 4.29-4.12 (m, 1H), 2.91-2.79 (m, 5H), 2.74-2.70 (m, 1H), 2.34 (s, 2H), 2.16-2.02 (m, 1H), 1.89-1.69 (m, 3H), 1.65 (d, J=16.80 Hz, 2H), 1.42 (s, 9H), 99.18% ee by chiral SFC (Rt=2.33 minute), Specific optical rotation: −46.2° [α]²⁰_{D}

The second eluting set of fractions was evaporated under pressure to afford tert-butyl 2-[1-[4-[[(3R)-2,6-dioxo-3-piperidyl]amino]-2-fluoro-phenyl]-4-hydroxy-4-piperidyl]acetate (530 mg, 1.17 mmol, 25.52% yield) as an off white solid. LCMS (ESI+) m/z: 436.0 [M+H]t 1H-NMR (400 MHz, DMSO-d₆): δ 10.78 (s, 1H), 6.84 (t, J=13.20 Hz, 1H), 6.49 (d, J=20.00 Hz, 1H), 6.41 (d, J=12.40 Hz, 1H), 5.77 (d, J=10.40 Hz, 1H), 4.44 (s, 1H), 4.27-4.22 (m, 1H), 2.92-2.77 (m, 5H), 2.73-2.63 (m, 1H), 2.34 (s, 2H), 2.18-2.03 (m, 1H), 1.87-1.73 (m, 3H), 1.64 (d, J=18.00 Hz, 2H), 1.48 (s, 9H), 99.13% ee by chiral SFC (Rt=4.92 minute), Specific optical rotation: +46.8° [α]²⁰_{D}

### Step 2: 2-[1-[4-[[(3S)-2,6-dioxo-3-piperidyl]amino]-2-fluoro-phenyl]-4-hydroxy-4-piperidyl]acetic acid hydrochloride

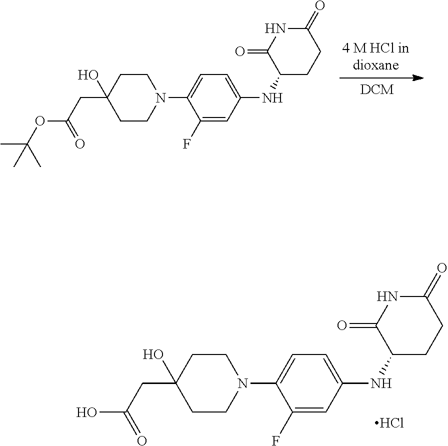 [SMILES: CC(C)(C)OC(=O)CC1(O)CCN(c2ccc(N[C@H]3CCC(=O)NC3=O)cc2F)CC1 ; O=C(O)CC1(O)CCN(c2ccc(N[C@H]3CCC(=O)NC3=O)cc2F)CC1 ; Cl]

To a stirred solution of tert-butyl 2-[1-[4-[[(3S)-2,6-dioxo-3-piperidyl]amino]-2-fluoro-phenyl]-4-hydroxy-4-piperidyl]acetate (600 mg, 1.38 mmol) in dichloromethane (15 mL) at 0° C. was added hydrogen chloride (4M solution in 1,4-dioxane, 1.72 mL, 6.89 mmol) dropwise. The reaction mixture was stirred at room temperature for 6 h. The volatiles were removed by rotary evaporation under reduced pressure. The residue was triturated twice with diethyl ether (2×10 ml). The solid residue was dried under vacuum to afford 2-[1-[4-[[(3S)-2,6-dioxo-3-piperidyl]amino]-2-fluoro-phenyl]-4-hydroxy-4-piperidyl]acetic acid hydrochloride (610 mg, 1.09 mmol, 78.96% yield) as a grey solid. LCMS (ESI+) m/z: 380.0 [M+H]⁺, ¹H-NMR (400 MHz, DMSO-d₆): δ 12.03 (bs, 1H), 10.86 (s, 1H), 7.63 (s, 1H), 6.70 (d, J=15.20 Hz, 1H), 6.58 (dd, J=11.40, 6.80 Hz, 1H), 4.43 (dd, J=11.60, 4.40 Hz, 1H), 3.88-3.65 (m, 5H), 3.41-3.36 (m, 2H), 2.74-2.68 (m, 1H), 2.59-2.54 (m, 1H), 2.46 (s, 2H), 2.33 (bs, 2H), 2.10-2.08 (m, 1H), 1.94-1.88 (m, 2H).

### Step 3: 2-(6,7-dihydro-5H-pyrrolo[1,2-c]imidazol-1-yl)-2-[6-[6-[2-[2-[1-[4-[[(3S)-2,6-dioxo-3-piperidyl]amino]-2-fluoro-phenyl]-4-hydroxy-4-piperidyl]acetyl]-2,6-diazaspiro[3.3]heptan-6-yl]-3-pyridyl]-4-fluoro-indazol-2-yl]-N-thiazol-2-yl-acetamide

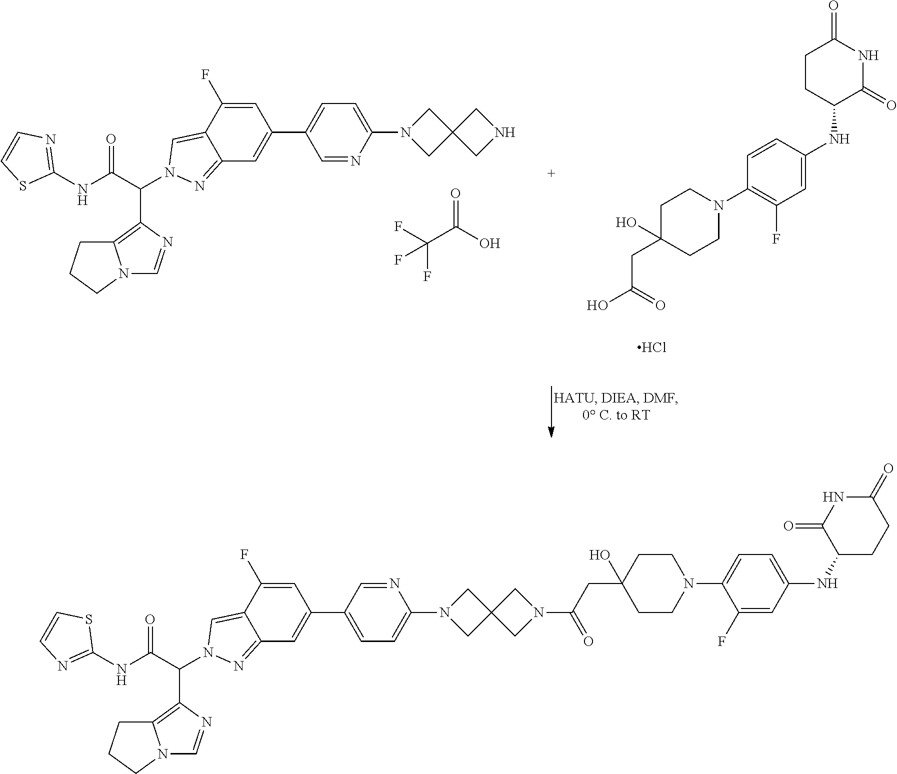

To a stirred solution of 2-[1-[4-[[(3S)-2,6-dioxo-3-piperidyl]amino]-2-fluoro-phenyl]-4-hydroxy-4-piperidyl]acetic acid hydrochloride (29.34 mg, 70.56 μmol) in N,N-dimethylformamide (3 mL) was added N,N-Diisopropylethylamine (69.48 mg, 537.59 μmol, 93.64 μL) at 0° C. 1-[Bis(dimethylamino)methylene]-1H-1,2,3-triazolo[4,5-b]pyridinium 3-oxid hexafluoro-phosphate (38.33 mg, 100.80 μmol) was added at the same temperature. 2-[6-[6-(2,6-diazaspiro[3.3]heptan-2-yl)-3-pyridyl]-4-fluoro-indazol-2-yl]-2-(6,7-dihydro-5H-pyrrolo[1,2-c]imidazol-1-yl)-N-thiazol-2-yl-acetamide, trifluoroacetic acid salt (45 mg, 67.20 μmol) was added, and the reaction mixture was stirred for 5 h while warming to room temperature. The reaction mixture was directly injected on a C18 column (120 g) for purification (0% to 60% acetonitrile in water (+0.1% ammonium acetate) over 45 minutes, then steep gradient to 100% acetonitrile). The pure fractions were frozen and lyophilized to afford product Compound 75 (38 mg, 40.98 μmol, 60.99% yield) as off white solid. LCMS (ESI+) m/z: 917.3 [M+H]t ¹H-NMR (400 MHz, DMSO-d₆): δ 12.83 (s, 1H), 10.79 (s, 1H), 8.51 (d, J=2.40 Hz, 1H), 8.28 (s, 1H), 7.95 (dd, J=8.80, 2.40 Hz, 1H), 7.68 (d, J=11.20 Hz, 1H), 7.51 (d, J=3.60 Hz, 1H), 7.29 (s, 1H), 7.16 (d, J=12.00 Hz, 1H), 6.85 (t, J=9.60 Hz, 1H), 6.70 (s, 1H), 6.50 (d, J=9.20 Hz, 2H), 6.45 (d, J=Hz, 1H), 5.79 (d, J=7.60 Hz, 1H), 4.77 (s, 1H), 4.39 (s, 1H), 4.29□4.21 (m, 1H), 4.14 (s, 4H), 4.12 (s, 2H), 4.09 (m, 2H), 2.85 (m, 7H), 2.51 (m, 2H), 2.22 (s, 2H), 2.09 (m, 1H), 1.87 (m, 1H), 1.80 (m, 2H), 1.62 (br d, J=12.8 Hz, 2H).

### Example 45. 2-(6,7-dihydro-5H-pyrrolo[1,2-c]imidazol-1-yl)-2-[6-[4-[2-[2-[1-[4-[[(3S)-2,6-dioxo-3-piperidyl]amino]-2-fluoro-phenyl]-4-hydroxy-4-piperidyl]acetyl]-2,6-diazaspiro[3.3]heptan-6-yl]phenyl]-4-fluoro-indazol-2-yl]-N-thiazol-2-yl-acetamide, (Compound 77)

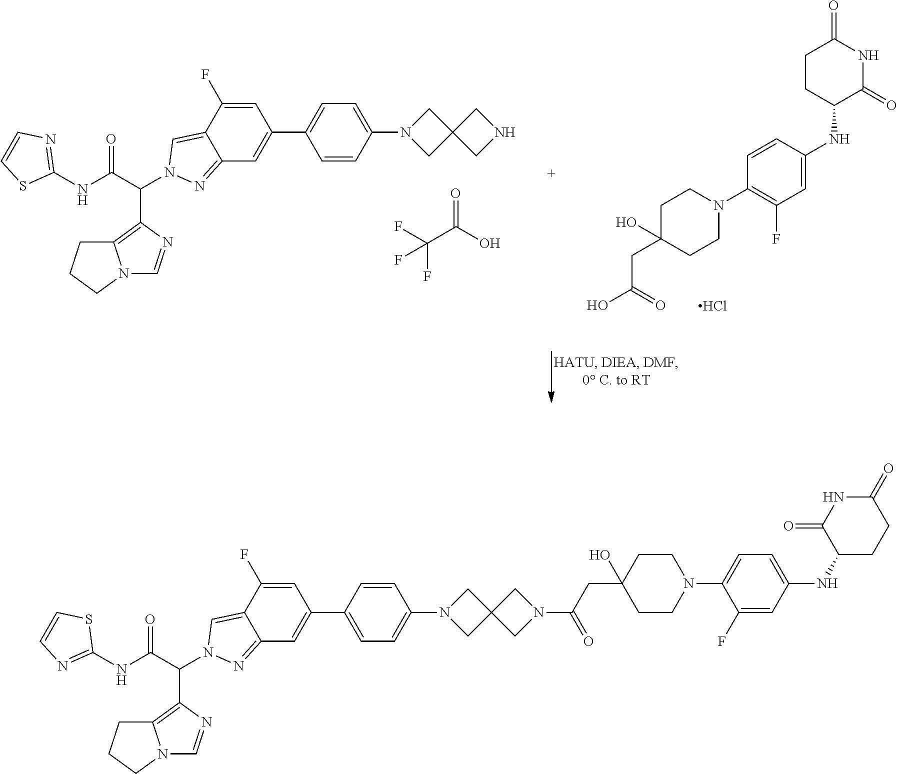

2-[6-[4-(2,6-diazaspiro[3.3]heptan-2-yl)phenyl]-4-fluoro-indazol-2-yl]-2-(6,7-dihydro-5H-pyrrolo[1,2-c]imidazol-1-yl)-N-thiazol-2-yl-acetamide, trifluoroacetic acid salt (300 mg, 448.66 μmol) and 2-[1-[4-[[(3S)-2,6-dioxo-3-piperidyl]amino]-2-fluoro-phenyl]-4-hydroxy-4-piperidyl]acetic acid hydrochloride (223.88 mg, 538.39 μmol) were mixed in N,N-dimethylformamide (2.5 mL). The reaction mixture was cooled to 0° C. and N,N-diisopropylethylamine (347.91 mg, 2.69 mmol, 468.89 μL) was added. HATU (221.77 mg, 583.25 μmol) was added, and stirred for 2 h while warming to room temperature. The reaction mixture was directly injected on a C-18 column (100 g) for purification (0-45% of acetonitrile in water+0.1% ammonium acetate over 30 minutes, then steep gradient to 100% acetonitrile). The pure fractions were combined and lyophilized to get Compound 77 (102 mg, 108.41 μmol, 24.16% yield) as an off white solid. LCMS (ESI+) m/z: 916.8 [M+H]⁺, ¹H-NMR (400 MHz, DMSO-d₆): δ 12.81 (s, 1H), 10.78 (s, 1H), 8.25 (s, 1H), 7.68 (s, 1H), 7.60 (d, J=8.40 Hz, 3H), 7.49 (d, J=2.80 Hz, 1H), 7.28-7.21 (m, 1H), 7.11 (d, J=12.40 Hz, 1H), 6.86 (t, J=9.60 Hz, 1H), 6.68-6.62 (m, 1H), 6.55-6.48 (m, 3H), 6.43-6.41 (m, 1H), 5.78 (d, J=7.60 Hz, 1H), 4.77 (s, 1H), 4.41-4.37 (m, 2H), 4.27-4.24 (m, 1H), 4.09-4.01 (m, 8H), 2.92-2.83 (m, 5H), 2.80-2.70 (m, 1H), 2.61-2.52 (m, 4H), 2.34-2.33 (m, 2H), 2.12-2.08 (m, 1H), 1.90-1.84 (m, 1H), 1.80-1.74 (m, 2H), 1.63 (s, 2H).

### Example 46. 2-(6,7-dihydro-5H-pyrrolo[1,2-c]imidazol-1-yl)-2-[6-[4-[4-[2-[1-[4-[(2,6-dioxo-3-piperidyl)amino]-2-fluoro-phenyl]-4-hydroxy-4-piperidyl]acetyl]piperazin-1-yl]phenyl]-4-fluoro-1-oxo-isoindolin-2-yl]-N-thiazol-2-yl-acetamide, Compound 83

### Step 1: 1-(2-fluoro-4-nitro-phenyl)piperidin-4-one

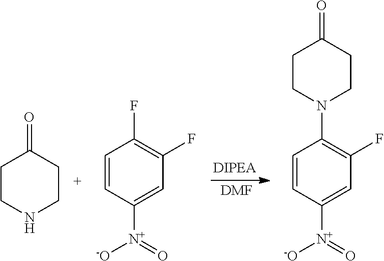

To a solution of piperidin-4-one (15.0 g, 151.31 mmol), 1,2-difluoro-4-nitro-benzene (24.07 g, 151.31 mmol, 16.72 mL) in N,N-dimethylformamide (30 mL) was added N,N-diisopropylethylamine (78.22 g, 605.26 mmol, 105.42 mL) and heated at 110° C. for 14 h. The reaction mixture was diluted with ethyl acetate (500 mL) and washed with cold water (150 mL). The organic layer was washed with a brine solution (150 mL), dried over sodium sulfate and concentrated under reduced pressure. The residue was purified by column chromatography on silica gel (40% ethyl acetate in petroleum ether) to afford 1-(2-fluoro-4-nitro-phenyl)piperidin-4-one (21 g, 77.93 mmol, 51.50% yield) as brown solid. LCMS, m/z: 238.9 [M+H]⁺

### Step 2: Synthesis of tert-butyl 2-[1-(4-amino-2-fluoro-phenyl)-4-hydroxy-4-piperidyl]acetate

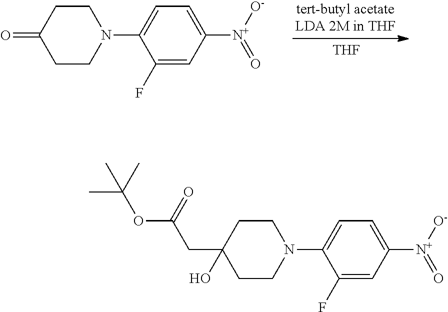 [SMILES: O=C1CCN(c2ccc([N+](=O)[O-])cc2F)CC1 ; CC(C)(C)OC(=O)CC1(O)CCN(c2ccc([N+](=O)[O-])cc2F)CC1 ; "THF"]

To a stirred solution of tert-butyl acetate (1.76 g, 15.11 mmol, 2.03 mL) in tetrahydrofuran (25 mL) was added dropwise lithium diisopropylamide (2 M in tetrahydrofuran, 12.59 mL) at −78° C. The reaction mixture was stirred at −78° C. for 45 minutes. 1-(2-fluoro-4-nitro-phenyl)piperidin-4-one (3 g, 12.59 mmol) dissolved in tetrahydrofuran (15 mL) was added at −78° C. The reaction mixture was stirred at −78° C. for 2 h. The reaction mixture was quenched with saturated ammonium chloride solution and the mixture was extracted with ethyl acetate. The organic layer was dried over sodium sulfate and concentrated under reduced pressure. The residue was purified by silica gel (100-200 mesh) column chromatography (eluent: 30% to 40% ethyl acetate in petroleum ether) to afford tert-butyl 2-[1-(2-fluoro-4-nitro-phenyl)-4-hydroxy-4-piperidyl]acetate (2.7 g, 7.01 mmol, 55.66% yield) as light yellow sticky solid. LCMS (355.1 (M+H)+)

### Step 3: tert-Butyl 2-[1-(4-amino-2-fluoro-phenyl)-4-hydroxy-4-piperidyl]acetate

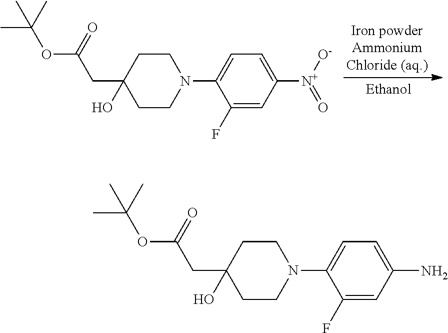 [SMILES: CC(C)(C)OC(=O)CC1(O)CCN(c2ccc([N+](=O)[O-])cc2F)CC1 ; CC(C)(C)OC(=O)CC1(O)CCN(c2ccc(N)cc2F)CC1]

To a solution of tert-butyl 2-[1-(2-fluoro-4-nitro-phenyl)-4-hydroxy-4-piperidyl]acetate (1.5 g, 4.23 mmol) in Ethanol (10 mL) and water (2 mL) were added iron powder (1.18 g, 21.16 mmol, 150.37 μL) and ammonium chloride (679.26 mg, 12.70 mmol, 443.96 μL). The reaction was stirred at 70° C. for 4 h. The reaction mixture was filtered through celite and the filter cake was washed with ethyl acetate (60 mL). The filtrate was washed with water (20 mL), aqueous sodium bicarbonate (20 mL) and brine (20 mL). The organic layer was dried over sodium sulfate and concentrated under reduced pressure to yield a residue, which was purified by column chromatography on silica gel, eluting with 70% ethyl acetate in petroleum ether to afford tert-butyl 2-[1-(4-amino-2-fluoro-phenyl)-4-hydroxy-4-piperidyl]acetate (1.2 g, 3.44 mmol, 81.28% yield) as brown sticky solid. LCMS m/z: 325.1 [M+H], ¹HNMR (DMSO-d₆) 8.02-7.89 (m, 2H), 7.23-7.05 (m, 1H), 4.69 (s, 1H), 3.55-3.43 (m, 2H), 3.22-3.19 (m, 2H), 2.36 (s, 2H), 1.88-1.64 (m, 3H), 1.41 (s, 9H).

### Step 4: tert-Butyl 2-[1-[4-[(2,6-dioxo-3-piperidyl)amino]-2-fluoro-phenyl]-4-hydroxy-4-piperidyl]acetate

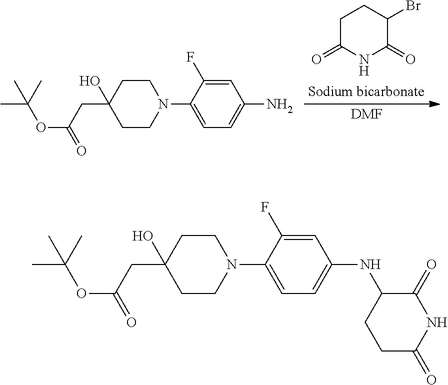

To a stirred solution of tert-butyl 2-[1-(4-amino-2-fluoro-phenyl)-4-hydroxy-4-piperidyl]acetate (1 g, 3.08 mmol) in N,N-dimethylformamide (10 mL) were added sodium bicarbonate (517.94 mg, 6.17 mmol, 239.79 μL) under nitrogen atmosphere in a 25 mL sealed tube. The vial was sealed and heated at 60° C. overnight. The reaction mixture was filtered through celite bed, washed 2 times with ethyl acetate and filtrate was concentrated under reduced pressure at 35° C. The crude residue was purified over silica column (100-200 mesh) eluting with 65-70% ethyl acetate:petroleum ether to afford tert-butyl 2-[1-[4-[(2,6-dioxo-3-piperidyl)amino]-2-fluoro-phenyl]-4-hydroxy-4-piperidyl]acetate (760 mg, 1.67 mmol, 54.11% yield) as an off white solid. LCMS m/z: 436.0 [M+H]. ¹H-NMR (DMSO-d₆): 10.79 (s, 1H), 6.87-6.80 (m, 1H), 6.52 (dd, J=13.6 Hz, 3.6 Hz, 1H), 6.41 (dd, J=3.7 Hz, 1.6 Hz, 1H), 4.89 (d, J=3.6 Hz, 1H), 4.45 (s, 1H), 4.30-4.19 (m, 1H), 2.90-2.80 (m, 4H), 2.78-2.51 (m, 3H), 2.49-2.41 (m, 1H), 2.13-2.01 (m, 2H), 1.95-1.63 (m, 4H), 1.42 (s, 9H).

### Step 5: 2-[1-[4-[(2,6-dioxo-3-piperidyl)amino]-2-fluoro-phenyl]-4-hydroxy-4-piperidyl]acetic acid hydrochloride

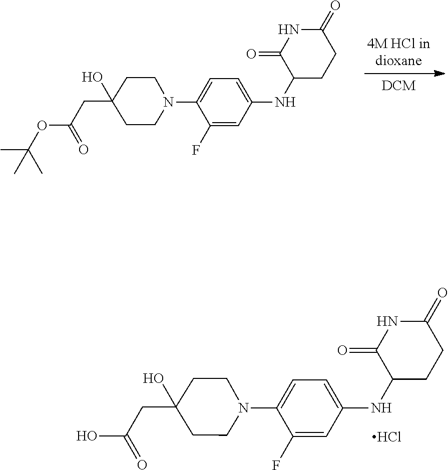 [SMILES: CC(C)(C)OC(=O)CC1(O)CCN(c2ccc(NC3CCC(=O)NC3=O)cc2F)CC1 ; O=C(O)CC1(O)CCN(c2ccc(NC3CCC(=O)NC3=O)cc2F)CC1 ; Cl]

To a stirred solution of tert-butyl 2-[1-[4-[(2,6-dioxo-3-piperidyl)amino]-2-fluoro-phenyl]-4-hydroxy-4-piperidyl]acetate (1.0 g, 2.30 mmol) in dichloromethane (10 mL) was added hydrogen chloride (4M in 1,4-dioxane, 400 mmol, 10 mL) dropwise at 0° C. The reaction mixture stirred at 25° C. for 3 h. The reaction mixture was concentrated under reduced pressure to give 2-[1-[4-[(2,6-dioxo-3-piperidyl)amino]-2-fluoro-phenyl]-4-hydroxy-4-piperidyl]acetic acid hydrochloride (900 mg, 2.16 mmol, 93.89% yield) as an off-white solid. LCMS m/z 380.2 (M+H)⁺.

### Step 6: 2-(6,7-dihydro-5H-pyrrolo[1,2-c]imidazol-1-yl)-2-[6-[4-[4-[2-[1-[4-[(2,6-dioxo-3-piperidyl)amino]-2-fluoro-phenyl]-4-hydroxy-4-piperidyl]acetyl]piperazin-1-yl]phenyl]-4-fluoro-1-oxo-isoindolin-2-yl]-N-thiazol-2-yl-acetamide

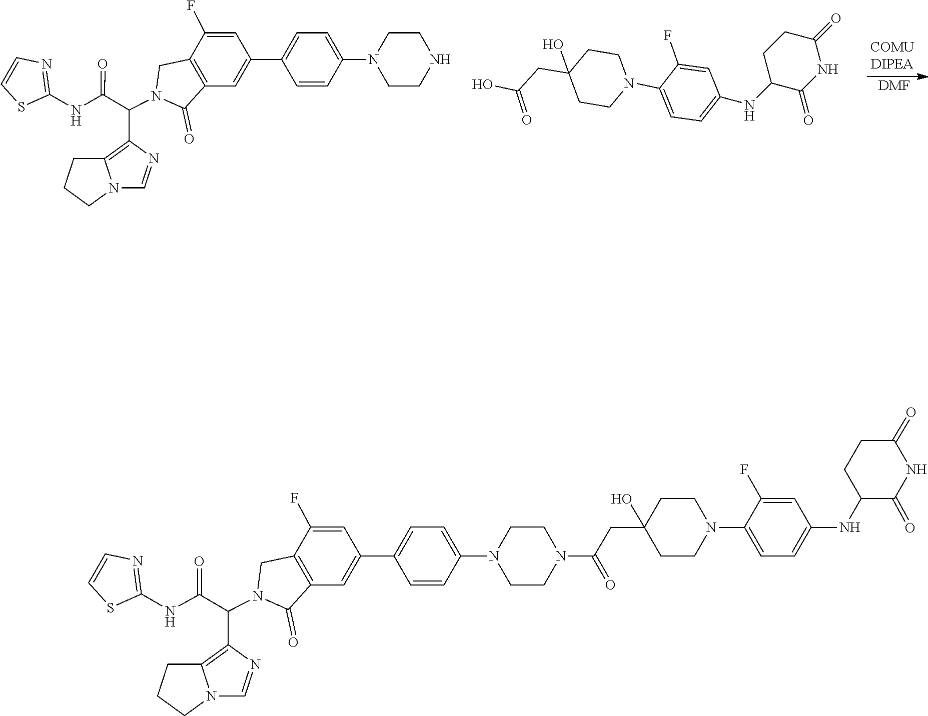

To a stirred solution of 2-[1-[4-[(2,6-dioxo-3-piperidyl)amino]-2-fluoro-phenyl]-4-hydroxy-4-piperidyl]acetic acid hydrochloride (115.49 mg, 277.73 μmol) in N,N-dimethylformamide (1.5 mL) in a round bottom flask was added N,N-diisopropylethylamine (195.79 mg, 1.51 mmol, 263.87 μL) dropwise at 0° C. Reaction mixture was stirred for 5 minutes. 1-[(1-(Cyano-2-ethoxy-2-oxoethylideneaminooxy)-dimethylamino-morpholino)] uronium hexafluorophosphate (324.39 mg, 757.45 μmol) was added, and the reaction mixture was stirred for 5 minutes. 2-(6,7-dihydro-5H-pyrrolo[1,2-c]imidazol-1-yl)-2-[4-fluoro-1-oxo-6-(4-piperazin-1-ylphenyl)isoindolin-2-yl]-N-thiazol-2-yl-acetamide hydrochloride (150 mg, 252.48 μmol) was added. The reaction was continued about 40 min at 0° C. Cold water was added to the reaction mixture, solid was precipitated, collected by filtration, washed with water and dried under suction. The precipitate was purified by preparative HPLC. Purification conditions: Column: Agilent C18 (50*21.2 mm), micron particle size. Mobile Phase: 10 mM ammonium acetate in water:acetonitrile. The collected pure fraction were lyophilized to afford Compound 83 as a pale yellow solid (47 mg, 51.00 μmol, 20.20% yield). LCMS (m/z: 917.3 (M−1)) ¹H-NMR (400 MHz, DMSO-d₆): δ 12.53 (s, 1H), 10.78 (s, 1H), 7.79-7.69 (m, 4H), 7.62 (s, 1H), 7.49 (d, J=3.60 Hz, 1H), 7.27 (d, J=3.60 Hz, 1H), 7.07 (d, J=8.80 Hz, 2H), 6.86 (t, J=9.60 Hz, 1H), 6.50 (dd, J=2.40, 14.80 Hz, 1H), 6.42 (dd, J=6.00, Hz, 1H), 6.16 (s, 1H), 5.78 (d, J=7.60 Hz, 1H), 4.83 (t, J=17.60 Hz, 2H), 4.25-4.21 (m, 2H), 4.02-3.96 (m, 2H), 3.73-3.68 (m, 4H), 3.44-3.34 (m, 2H), 3.44-3.23 (m, 5H), 2.91-2.84 (m, 4H), 2.78-2.67 (m, 2H), 2.58-2.52 (m, 3H), 2.13-2.02 (m, 1H), 1.89-1.65 (m, 5H).

### Example 47. 2-(6,7-dihydro-5H-pyrrolo[1,2-c]imidazol-1-yl)-2-[6-[4-[1-[2-[4-[3-(2,4-dioxohexahydropyrimidin-1-yl)-1-methyl-indazol-6-yl]-1-piperidyl]-2-oxo-ethyl]-4-piperidyl]phenyl]-4-fluoro-1-oxo-isoindolin-2-yl]-N-thiazol-2-yl-acetamide, (Compound 85)

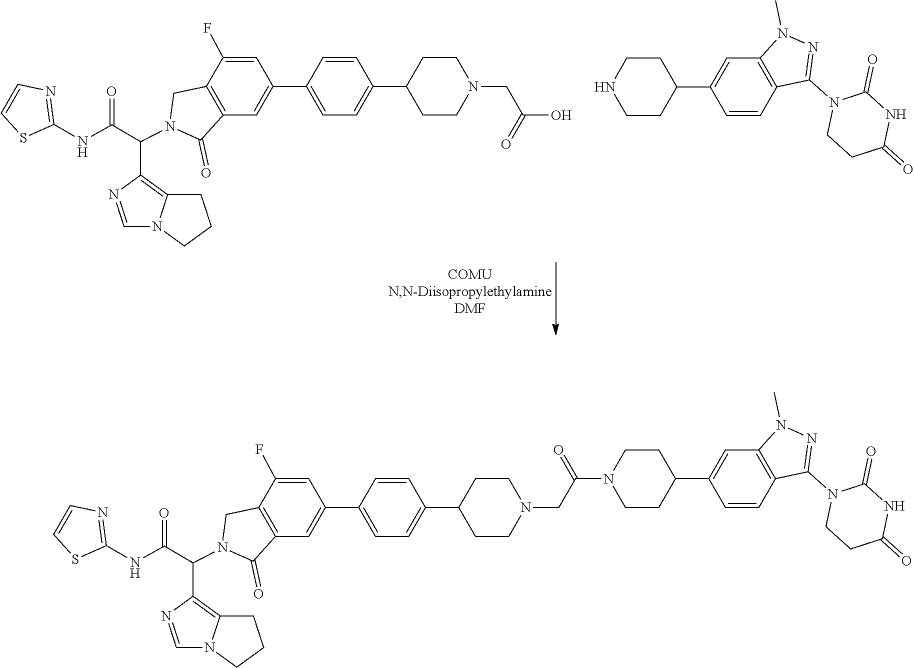 [SMILES: O=C(O)CN1CCC(c2ccc(-c3cc(F)c4c(c3)C(=O)N(C(C(=O)Nc3nccs3)c3ncn5c3CCC5)C4)cc2)CC1 ; Cn1nc(N2CCC(=O)NC2=O)c2ccc(C3CCNCC3)cc21 ; Cn1nc(N2CCC(=O)NC2=O)c2ccc(C3CCN(C(=O)CN4CCC(c5ccc(-c6cc(F)c7c(c6)C(=O)N(C(C(=O)Nc6nccs6)c6ncn8c6CCC8)C7)cc5)CC4)CC3)cc21]

To a solution of 2-[4-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]-1-piperidyl]acetic acid hydrochloride (130 mg, 199.65 μmol) in N,N-dimethylformamide (1.5 mL), N,N-diisopropylethylamine (129.01 mg, 998.23 μmol, 173.87 μL) and 1-[(1-(cyano-2-ethoxy-2-oxoethylideneaminooxy)-dimethylamino-morpholino)] uronium hexafluorophosphate (171.01 mg, 399.30 μmol) were added at 0° C. The reaction mixture was stirred for 15 minutes. 1-[1-methyl-6-(4-piperidyl)indazol-3-yl]hexahydropyrimidine-2,4-dione hydrochloride (72.64 mg, 199.65 μmol) was added. The reaction mixture was stirred for 1 h. The reaction mixture was concentrated under reduced pressure. The residue was purified by reverse phase chromatography (C18 column, 0-100% of 0.1% ammonium acetate in water and acetonitrile). The fractions containing compound were frozen and lyophilized. The residue purified by reverse phase prep HPLC using Column: Zorbax Extend C18(50×4.6 mm) 5 μm, Mobile Phase A: 10 mM ammonium acetate in water, Mobile Phase B: acetonitrile. Pure fractions were lyophilized to get Compound 85 (4.81 mg, 5.07 μmol, 2.54% yield) as a white solid which was submitted for analysis. LCMS (ESI+): 924.3 (M+H); 1H-NMR (400 MHz, DMSO-d₆): 12.50 (s, 1H), 10.55 (d, J=10.0 Hz, 1H), 7.81 (d, J=5.6 Hz, 2H), 7.73 (d, J=8.0 Hz, 2H), 7.63 (s, 1H), 7.59 (d, J=8.40 Hz, 1H), 7.49 (d, J=3.6 Hz, 1H), 7.45 (s, 1H), 7.37 (d, J=8.4 Hz, 2H), 7.26 (d, J=3.2 Hz, 1H), 7.05 (d, J=8.4 Hz, 1H), 6.15 (s, 1H), 4.83 (d, J=17.6 Hz, 1H), 4.61-4.53 (m, 1H), 4.27-4.22 (m, 2H), 4.01-3.91 (m, 7H), 3.34-3.17 (m, 3H), 3.00-2.97 (m, 5H), 2.78-2.75 (m, 4H), 2.33-2.17 (m, 3H), 1.91-1.70 (m, 9H).

### Example 48. 2-(6,7-dihydro-5H-pyrrolo[1,2-c]imidazol-1-yl)-2-[6-[4-[4-[2-[4-[4-[(2,6-dioxo-3-piperidyl)amino]-2-fluoro-phenyl]-1-piperidyl]-2-oxo-ethyl]-1-piperidyl]phenyl]-4-fluoro-1-oxo-isoindolin-2-yl]-N-thiazol-2-yl-acetamide, (Compound 86)

### Step 1: methyl 2-(1-(4-(2-(1-(6,7-dihydro-5H-pyrrolo[1,2-c]imidazol-1-yl)-2-oxo-2-(thiazol-2-ylamino)ethyl)-7-fluoro-3-oxoisoindolin-5-yl)phenyl)piperidin-4-yl)acetate

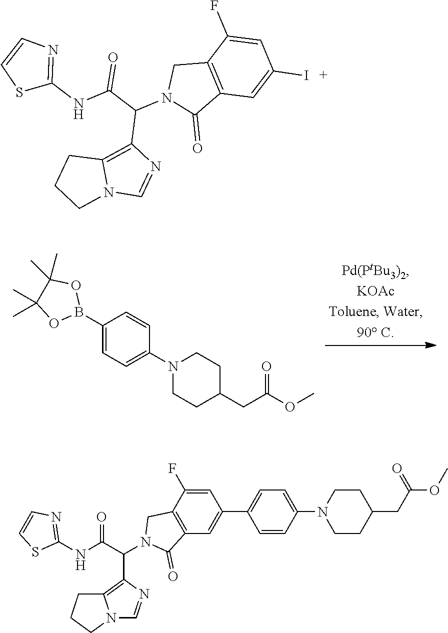

Into a 100 mL sealed tube containing a well-stirred solution of 2-(6,7-dihydro-5H-pyrrolo[1,2-c]imidazol-1-yl)-2-(4-fluoro-6-iodo-1-oxo-isoindolin-2-yl)-N-thiazol-2-yl-acetamide (400 mg, 764.35 μmol) and methyl 2-[1-[4-(4,4,5,5-tetramethyl-1,3,2-dioxaborolan-2-yl)phenyl]-4-piperidyl]acetate (411.91 mg, 1.15 mmol) in anhydrous toluene (5 mL) and water (1.5 mL) was added potassium acetate (225.04 mg, 2.29 mmol, 143.34 μL) at the ambient temperature under nitrogen atmosphere. The resulting mixture was degassed with N₂ for 10 minutes. Bis(tri-tert-butylphosphine) palladium (0) (78.12 mg, 152.87 μmol) was added and the reaction mixture was stirred at 95° C. for 64 h. The reaction mixture was diluted with ethyl acetate and filtered through celite. The filtrate was evaporated under reduced pressure. The crude residue was triturated with diethyl ether to afford methyl 2-[1-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]-4-piperidyl]acetate (205 mg, 295 μmol, 42.7% yield). LCMS (ESI+) m/z: 629.2 [M+H]⁺.

### Step 2: 2-[1-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]-4-piperidyl]acetic acid

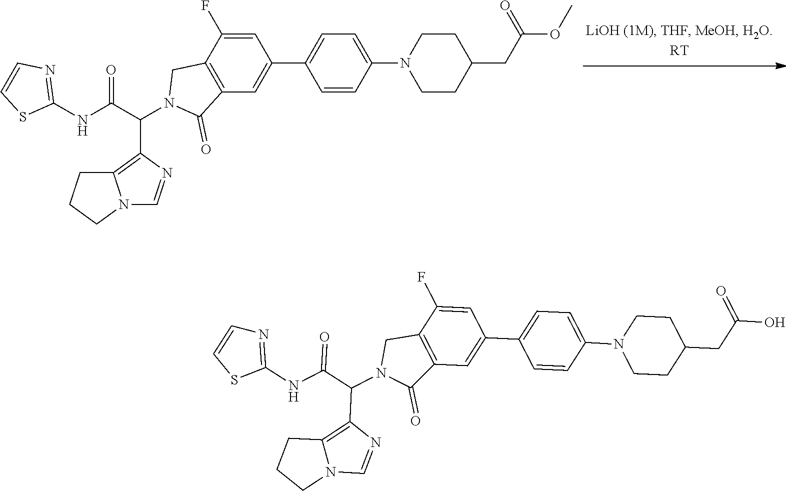 [SMILES: COC(=O)CC1CCN(c2ccc(-c3cc(F)c4c(c3)C(=O)N(C(C(=O)Nc3nccs3)c3ncn5c3CCC5)C4)cc2)CC1 ; O=C(O)CC1CCN(c2ccc(-c3cc(F)c4c(c3)C(=O)N(C(C(=O)Nc3nccs3)c3ncn5c3CCC5)C4)cc2)CC1]

To a solution of methyl 2-[1-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]-4-piperidyl]acetate (200 mg, 318.11 μmol) in tetrahydrofuran (2 mL) and methanol (2 mL) and water (2 mL) was added a lithium hydroxide (1M aqueous solution, 318 μL, 318.11 μmol) at 0° C. Reaction mixture was stirred for 3 hr at room temperature. Reaction mixture was concentrated to get crude, which was further dissolved in 5 mL of water and acidified using aqueous sodium bisulfate (pH 5-6). The solid precipitated was filtered to get 2-[1-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]-4-piperidyl]acetic acid (150 mg, 244.03 μmol, 39.9% yield). LCMS (ESI+) m/z: 615.2 [M+H]t

### Step 3: 2-(6,7-dihydro-5H-pyrrolo[1,2-c]imidazol-1-yl)-2-[6-[4-[4-[2-[4-[4-[(2,6-dioxo-3-piperidyl)amino]-2-fluoro-phenyl]-1-piperidyl]-2-oxo-ethyl]-1-piperidyl]phenyl]-4-fluoro-1-oxo-isoindolin-2-yl]-N-thiazol-2-yl-acetamide

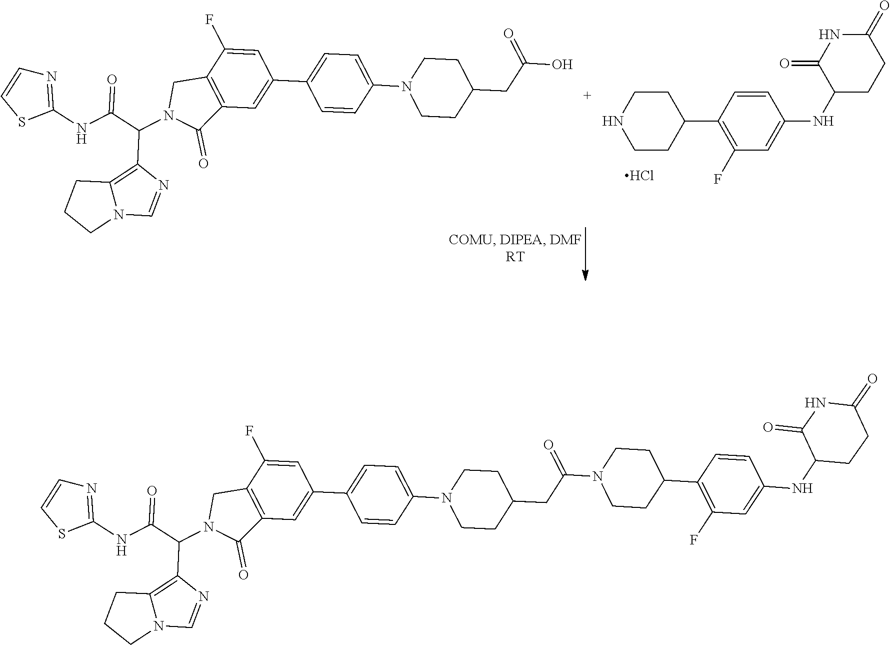

To a solution of 2-[1-[4-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]phenyl]-4-piperidyl]acetic acid (150 mg, 244.03 μmol) in N,N-dimethylformamide (2 mL) was added N,Ndiisopropylethylamine (157.69 mg, 1.22 mmol, 212.52 μL) and 1-[(1-(cyano-2-ethoxy-2-oxoethylideneaminooxy)-dimethylamino-morpholino)] uronium hexafluorophosphate (209.02 mg, 488.05 μmol) at 0° C. 3-[3-fluoro-4-(4-piperidyl) anilino] piperidine-2,6-dione hydrochloride (74.51 mg, 218.00 μmol) was added and stirred for 1 h. The crude was purified by reverse phase chromatography (C18 column, 0-100% of 0.1% Formic acid in water and acetonitrile). Collected fraction were lyophilized. The residue was purified by reverse phase preparative HPLC using Column: Zorbax Extend C18 (50×4.6 mm), 5 μm, (Mobile Phase A: 10 mM ammonium acetate in milli-q water; Mobile phase B: acetonitrile). The pure fractions were frozen and lyophilized to get Compound 86 (4.57 mg, 5.04 μmol, 2.06% yield) as a white solid. LCMS (ESI+) m/z: 902.3 [M+H]t 1H-NMR (400 MHz, DMSO-d₆): δ 12.61 (s, 1H), 10.80 (s, 1H), 7.76 (s, 1H), 7.73 (d, J=10.80 Hz, 1H), 7.65 (d, J=8.80 Hz, 2H), 7.61 (s, 1H), 7.48 (s, 1H), 7.25 (s, 1H), 7.02 (d, J=6.40 Hz, 2H), 6.97 (d, J=8.80 Hz, 1H), 6.47 (s, 1H), 6.44 (d, J=4.00 Hz, 1H), 6.14 (s, 1H), 6.04 (d, J=8.00 Hz, 1H), 4.81 (d, J=17.20 Hz, 1H), 4.60 (d, J=8.40 Hz, 1H), 4.32 (m, 1H), 4.33 (d, J=16.00 Hz, 1H), 4.20-3.99 (m, 3H), 3.81 (d, J=12.00 Hz, 2H), 3.11 (t, J=13.20 Hz, 1H), 2.87 (t, J=10.40 Hz, 1H), 2.79-2.71 (m, 4H), 2.68-2.61 (m, 3H), 2.33 (d, J=6.80 Hz, 2H), 2.09-2.07 (m, 1H), 1.92-1.90 (m, 2H), 1.88-1.71 (m, 5H), 1.68-1.32 (m, 2H), 1.31-1.24 (m, 3H).

### Example 49. 2-(6,7-dihydro-5H-pyrrolo[1,2-c]imidazol-1-yl)-2-(6-(6-(6-(2-(4-(3-(2,4-dioxotetrahydropyrimidin-1(2H)-yl)-1-methyl-1H-indazol-6-yl)-3,3-difluoropiperidin-1-yl)acetyl)-2,6-diazaspiro[3.3]heptan-2-yl)pyridin-3-yl)-4-fluoro-1-oxoisoindolin-2-yl)-N-(thiazol-2-yl)acetamide, (Compound 87)

### Step 1: tert-Butyl 6-(5-bromo-2-pyridyl)-2,6-diazaspiro[3.3]heptane-2-carboxylate

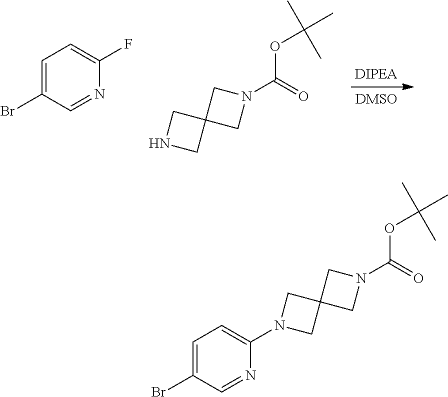 [SMILES: Fc1ccc(Br)cn1 ; CC(C)(C)OC(=O)N1CC2(CNC2)C1 ; CC(C)(C)OC(=O)N1CC2(C1)CN(c1ccc(Br)cn1)C2]

To a stirred solution of tert-butyl 2,6-diazaspiro[3.3]heptane-2-carboxylate (1.69 g, 8.52 mmol) in dimethylsulfoxide (10 mL) were added N,N-diisopropylethylamine (5.51 g, 42.62 mmol, 7.42 mL) and 5-bromo-2-fluoro-pyridine (1.5 g, 8.52 mmol, 877.19 μL). The reaction mixture was heated at 90° C. for 4 h. The reaction mixture was cooled to room temperature and quenched in crushed ice. Solid precipitated and it was filtered and dried to afford tert-butyl 6-(5-bromo-2-pyridyl)-2,6-diazaspiro[3.3]heptane-2-carboxylate (1.5 g, 4.14 mmol, 48.57% yield) as a white solid. LCMS (m/z: 356.1 [M+1]).

### Step 2: [6-(2-tert-Butoxycarbonyl-2,6-diazaspiro[3.3]heptan-6-yl)-3-pyridyl]boronic acid

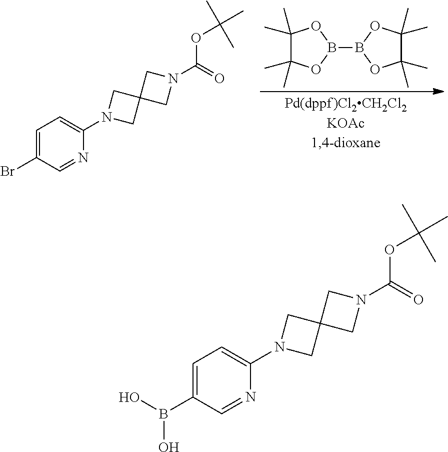

In a sealed tube to a stirred solution of tert-butyl 6-(5-bromo-2-pyridyl)-2,6-diazaspiro[3.3]heptane-2-carboxylate (650 mg, 1.83 mmol) in 1,4-dioxane (7 mL) was added 4,4,5,5-tetramethyl-2-(4,4,5,5-tetramethyl-1,3,2-dioxaborolan-2-yl)-1,3,2-dioxaborolane (698.93 mg, 2.75 mmol) followed by potassium acetate (360.16 mg, 3.67 mmol, 229.40 μL) added to the reaction mixture. The reaction mixture was degassed with nitrogen for 20 mins, followed by Pd(dppf)₂Cl₂·CH₂Cl₂ (449.52 mg, 550.47 μmol) added to the reaction mixture and degassed with nitrogen for 10 mins. The reaction mixture was heated at 90° C. on a heating block for 16 h. The reaction mixture was cooled to ambient temperature, the mixture was filtered through a pad of celite and washed with ethyl acetate. The filtrate was evaporated under reduced pressure afford [6-(2-tert-butoxy carbonyl-2,6-diazaspiro[3.3]heptan-6-yl)-3-pyridyl]boronic acid (810 mg, 1.14 mmol, 62.17% yield) which was submitted for analysis. LCMS data (m/z: 320.2 [M+1]).

### Step 3: tert-Butyl 6-[5-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]-2-pyridyl]-2,6-diazaspiro[3.3]heptane-2-carboxylate

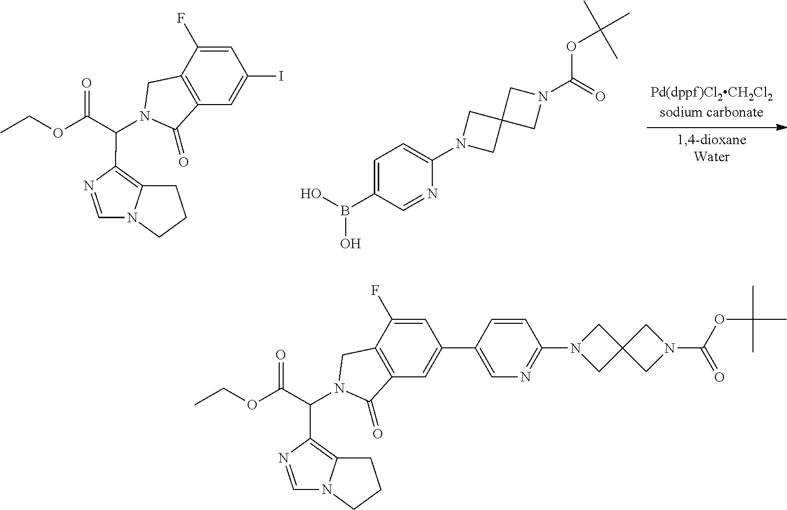 [SMILES: CCOC(=O)C(c1ncn2c1CCC2)N1Cc2c(F)cc(I)cc2C1=O ; CC(C)(C)OC(=O)N1CC2(C1)CN(c1ccc(B(O)O)cn1)C2 ; CCOC(=O)C(c1ncn2c1CCC2)N1Cc2c(F)cc(-c3ccc(N4CC5(CN(C(=O)OC(C)(C)C)C5)C4)nc3)cc2C1=O]

A solution of 2-(6,7-dihydro-5H-pyrrolo[1,2-c]imidazol-1-yl)-2-(4-fluoro-6-iodo-1-oxo-isoindolin-2-yl)-N-thiazol-2-yl-acetamide (350 mg, 668.80 μmol) and [6-(2-tert-butoxycarbonyl-2,6-diazaspiro[3.3]heptan-6-yl)-3-pyridyl]boronic acid (298.84 mg, 936.33 μmol) in 1,4-dioxane (8 mL) and water (2 mL) was degassed with nitrogen for 15 minutes. Sodium carbonate (212.66 mg, 2.01 mmol) and [1,1′-Bis(diphenylphosphino)ferrocene]dichloropalladium(II), complex with dichloromethane (87.02 mg, 106.55 μmol) were added to the reaction mixture and purged with nitrogen gas for 5 mins. The reaction mixture was heated at 80° C. under nitrogen for 16 h. The reaction mixture poured to ice water and the solid was filtered and the solid washed with water and dried. The crude was purified by silica gel column chromatography (0-7% Dichloromethane and Methanol) to get tert-butyl 6-[5-[2-[1-(6,7-dihydro-5H-pyrrolo[1,2-c]imidazol-1-yl)-2-oxo-2-(thiazol-2-yl amino)ethyl]-7-fluoro-3-oxo-isoindolin-5-yl]-2-pyridyl]-2,6-diazaspiro[3.3]heptane-2-carboxylate (160 mg, 224.60 μmol, 34% yield) as a brown solid. LCMS m/z 671.3 (M+H)⁺.

### Step 4: [2-[6-[6-(2-tert-butoxycarbonyl-2,6-diazaspiro[3.3]heptan-6-yl)-3-pyridyl]-4-fluoro-1-oxo-isoindolin-2-yl]-2-(6,7-dihydro-5H-pyrrolo[1,2-c]imidazol-1-yl)acetyl]oxylithium

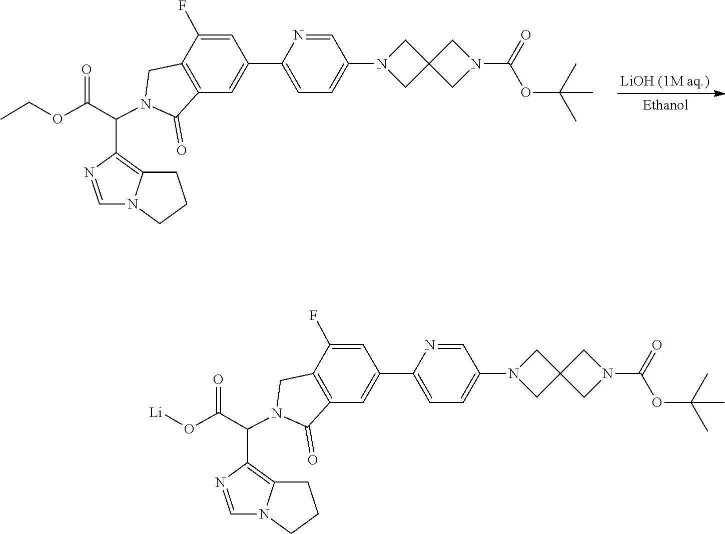

To a solution of tert-butyl 6-[5-[2-[1-(6,7-dihydro-5H-pyrrolo[1,2-c]imidazol-1-yl)-2-ethoxy-2-oxo-ethyl]-7-fluoro-3-oxo-isoindolin-5-yl]-2-pyridyl]-2,6-diazaspiro[3.3]heptane-2-carboxylate (440 mg, 713.50 μmol) in ethanol (3.2 mL) was added a lithium hydroxide aqueous solution (1 M, 784.85 μL) and stirred at 22° C. for 1 h. The volatiles were evaporated under reduced pressure to afford [2-[6-[6-(2-tert-butoxycarbonyl-2,6-diazaspiro[3.3]heptan-6-yl)-3-pyridyl]-4-fluoro-1-oxo-isoindolin-2-yl]-2-(6,7-dihydro-5H-pyrrolo[1,2-c]imidazol-1-yl)acetyl]oxylithium (420 mg, 698 μmol, 98% yield) LCMS: 598.2 (M+H)

### Step 5: Synthesis of tert-butyl 6-(6-(2-(1-(6,7-dihydro-5H-pyrrolo[1,2-c]imidazol-1-yl)-2-oxo-2-(thiazol-2-ylamino)ethyl)-7-fluoro-3-oxoisoindolin-5-yl)pyridin-3-yl)-2,6-diazaspiro[3.3]heptane-2-carboxylate

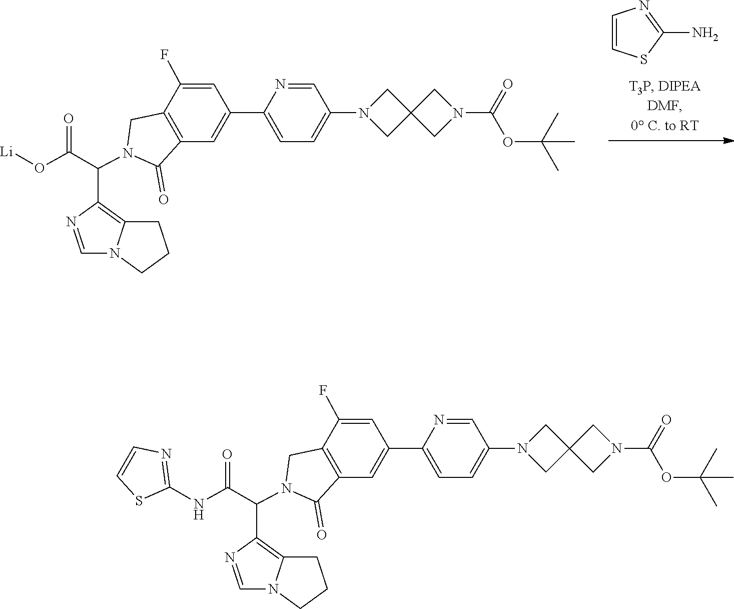

Into a 50 mL single-necked round-bottomed flask containing a well-stirred solution of [2-[6-[6-(2-tert-butoxycarbonyl-2,6-diazaspiro[3.3]heptan-6-yl)-3-pyridyl]-4-fluoro-1-oxo-isoindolin-2-yl]-2-(6,7-dihydro-5H-pyrrolo[1,2-c]imidazol-1-yl)acetyl]oxylithium (400.00 mg, 672.77 μmol) and 2-thiazole amine (87.58 mg, 874.59 μmol) in N,N-dimethylformamide (2.5 mL) was added N,N-diisopropylethylamine (542.95 mg, 672.77 μmol, 117.18 μL) under nitrogen atmosphere at 0° C. Propylphosphonic anhydride solution (50 wt. % in ethyl acetate) (521 μL, 874.59 μmol) was added at 0° C. and the resulting reaction mixture was stirred at room temperature for 4 h. The reaction mixture was added ice cold water and solid precipitated was filtered and dried under reduced pressure to afford tert-butyl 6-[5-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]-2-pyridyl]-2,6-diazaspiro[3.3]heptane-2-carboxylate (290 mg, 380.47 μmol, 56.55% yield) as an off-white solid. LCMS (ESI+) m/z: 671.3 [M+H]⁺.

### Step 6: Synthesis of 2-(6-(6-(2,6-diazaspiro[3.3]heptan-2-yl)pyridin-3-yl)-4-fluoro-1-oxoisoindolin-2-yl)-2-(6,7-dihydro-5H-pyrrolo[1,2-c]imidazol-1-yl)-N-(thiazol-2-yl)acetamide

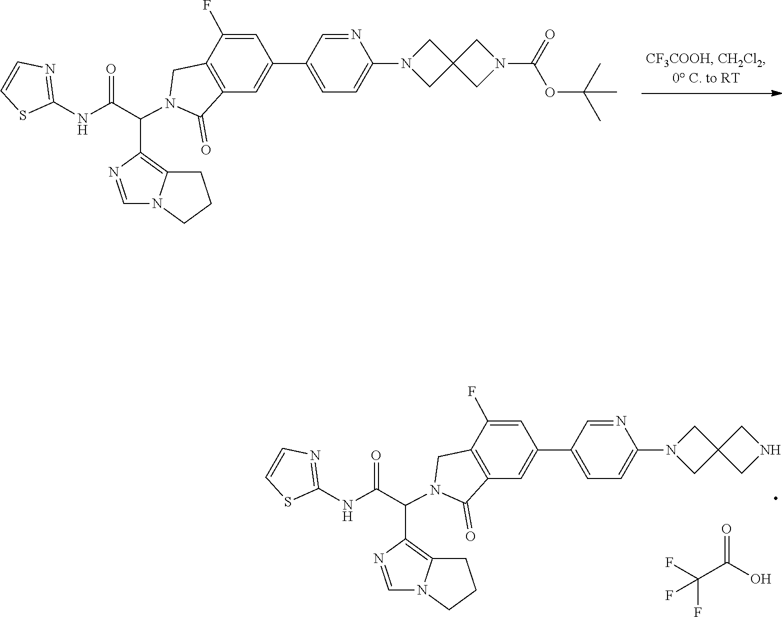 [SMILES: CC(C)(C)OC(=O)N1CC2(C1)CN(c1ccc(-c3cc(F)c4c(c3)C(=O)N(C(C(=O)Nc3nccs3)c3ncn5c3CCC5)C4)cn1)C2 ; O=C(Nc1nccs1)C(c1ncn2c1CCC2)N1Cc2c(F)cc(-c3ccc(N4CC5(CNC5)C4)nc3)cc2C1=O ; O=C(O)C(F)(F)F]

Into a 25 mL single-necked round-bottomed flask containing a well-stirred solution of tert-butyl 6-[5-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]-2-pyridyl]-2,6-diazaspiro[3.3]heptane-2-carboxylate (280.00 mg, 417.44 μmol) in dichloromethane (3.0 mL) was added trifluoroacetic acid (475.97 mg, 4.17 mmol, 321.60 μL) at 0° C. The mixture was stirred at ambient temperature for 3 h. The reaction mixture was concentrated under reduced pressure to afford crude, co-distilled with dichloromethane, triturated with diethyl ether and decanted to afford 2-[6-[6-(2,6-diazaspiro[3.3]heptan-2-yl)-3-pyridyl]-4-fluoro-1-oxo-isoindolin-2-yl]-2-(6,7-dihydro-5H-pyrrolo[1,2-c]imidazol-1-yl)-N-thiazol-2-yl-acetamide (270 mg, 345.06 μmol, 82.66% yield) as an off white solid. LCMS (ESI+) m/z: 571.2 [M+H]⁺.

### Step 7: Synthesis of 2-(6,7-dihydro-5H-pyrrolo[1,2-c]imidazol-1-yl)-2-(6-(6-(6-(2-(4-(3-(2,4-dioxotetrahydropyrimidin-1(2H)-yl)-1-methyl-1H-indazol-6-yl)-3,3-difluoropiperidin-1-yl)acetyl)-2,6-diazaspiro[3.3]heptan-2-yl)pyridin-3-yl)-4-fluoro-1-oxoisoindolin-2-yl)-N-(thiazol-2-yl)acetamide

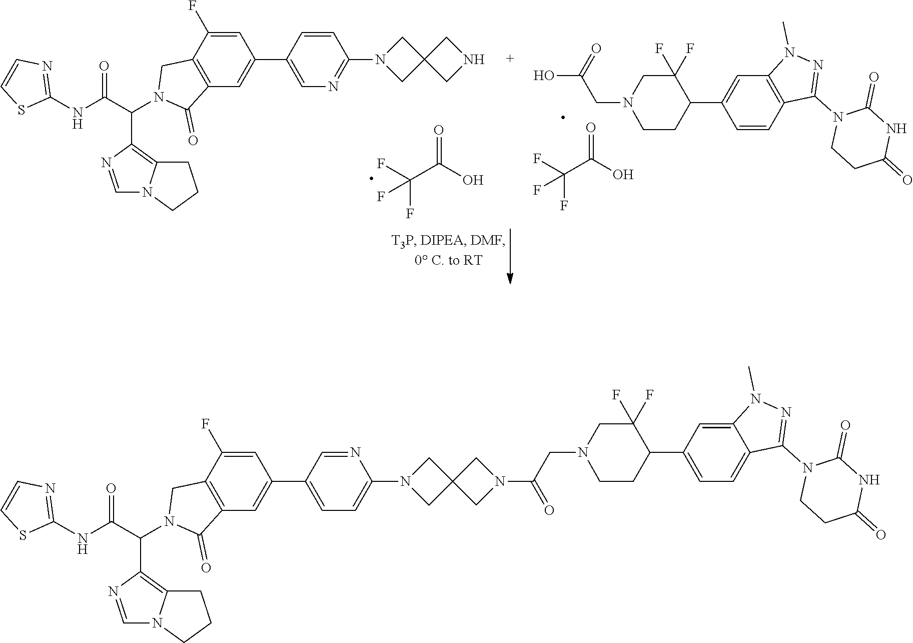

Into a 10 mL single-necked round-bottomed flask containing a well-stirred solution of 2-[6-[6-(2,6-diazaspiro[3.3]heptan-2-yl)-3-pyridyl]-4-fluoro-1-oxo-isoindolin-2-yl]-2-(6,7-dihydro-5H-pyrrolo[1,2-c]imidazol-1-yl)-N-thiazol-2-yl-acetamide; 2,2,2-trifluoroacetic acid (120 mg, 175.27 μmol), and 2-[4-[3-(2,4-dioxohexahydropyrimidin-1-yl)-1-methyl-indazol-6-yl]-3,3-difluoro-1-piperidyl]acetic acid; 2,2,2-trifluoroacetic acid (80.25 mg, 175.27 μmol) in N,N-dimethylformamide (4 mL) was added N, N-diisopropylethylamine (113.26 mg, 876.34 μmol, 152.64 μL) at 0° C. Propylphosphonic anhydride solution (50 wt. % in ethyl acetate) (139.42 mg, 438.17 μmol) was added to the reaction mixture at the same temperature and stirred while warming to room temperature for 1 h. The crude mixture was directly injected on a C18 column (100 g) for purification while eluting (0%-50% of acetonitrile in water+0.1% ammonium acetate over 30 minutes, followed by a steep gradient to 100% acetonitrile). The pure fraction was frozen and lyophilized to afford Compound 87 (48 mg, 48.95 μmol, 27.93% yield) as an off-white solid. LCMS (ESI+) m/z: 975.3[M+H]⁺. 1H-NMR (400 MHz, DMSO-d₆): δ 12.53 (s, 1H), 10.58 (s, 1H), 8.54 (d, J=2.40 Hz, 1H), 8.00 (dd, J=8.60, 2.40 Hz, 1H), 7.80 (s, 1H), 7.77 (s, 1H), 7.62 (s, 1H), 7.60 (d, J=8.80 Hz, 1H), 7.57 (s, 1H), 7.49 (d, J=3.60 Hz, 1H), 7.27 (d, J=3.60 Hz, 1H), 7.10 (d, J=8.40 Hz, 1H), 6.52 (d, J=8.40 Hz, 1H), 6.15 (s, 1H), 4.81 (d, J=18.00 Hz, 1H), 4.45 (s, 2H), 4.25-4.15 (m, 6H), 4.12 (s, 2H), 4.02-4.00 (m, 5H), 3.93 (t, J=6.80 Hz, 3H), 3.21 (d, J=8.40 Hz, 4H), 3.01-2.98 (m, 1H), 2.78 (t, J=6.40 Hz, 4H), 2.51-2.50 (m, 2H), 2.35-2.30 (m, 1H), 1.89-1.80 (m, 1H).

### Example 50. 5-[2-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]ethynyl]-N-[1-[2-[4-[3-(2,4-dioxohexahydropyrimidin-1-yl)-1-methyl-indazol-6-yl]-3,3-difluoro-1-piperidyl]acetyl]-4-piperidyl]pyridine-2-carboxamide, (Compound 88)

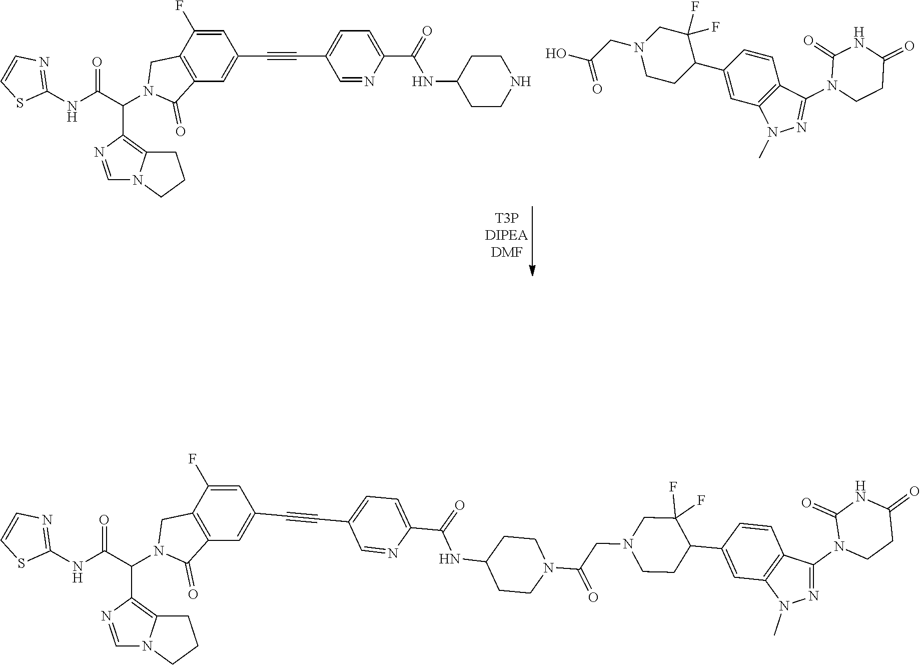 [SMILES: O=C(NC1CCNCC1)c1ccc(C#Cc2cc(F)c3c(c2)C(=O)N(C(C(=O)Nc2nccs2)c2ncn4c2CCC4)C3)cn1 ; Cn1nc(N2CCC(=O)NC2=O)c2ccc(C3CCN(CC(=O)O)CC3(F)F)cc21 ; Cn1nc(N2CCC(=O)NC2=O)c2ccc(C3CCN(CC(=O)N4CCC(NC(=O)c5ccc(C#Cc6cc(F)c7c(c6)C(=O)N(C(C(=O)Nc6nccs6)c6ncn8c6CCC8)C7)cn5)CC4)CC3(F)F)cc21]

To the stirred solution of 5-[2-[2-[1-(6,7-dihydro-5H-pyrrolo[1,2-c]imidazol-1-yl)-2-oxo-2-(thiazol-2-ylamino)ethyl]-7-fluoro-3-oxo-isoindolin-5-yl]ethynyl]-N-(4-piperidyl)pyridine-2-carboxamide hydrochloride (100 mg, 151.25 μmol) and 2-[4-[3-(2,4-dioxohexahydropyrimidin-1-yl)-1-methyl-indazol-6-yl]-3,3-difluoro-1-piperidyl]acetic acid, trifluoroacetic acid salt (80.98 mg, 151.25 μmol) in N,N-dimethylformamide (2 mL) was cooled to 0° C. N,N-diisopropylethylamine (117.29 mg, 907.51 μmol, 158.07 μL) was added to the reaction mixture followed by propanephosphonic acid anhydride (50% in N,N-dimethylformamide) (96.25 mg, 302.50 μmol) at 0° C. The reaction mixture stirred at ambient temperature for 1 h. The reaction mixture was directly injected on a C18 column (100 g) for purification (0-50%, water (0.1% ammonium acetate) in Acetonitrile over 30 minutes, followed by steep gradient to 100% acetonitrile). The pure fractions were combined and lyophilized to get Compound 88 (93 mg, 89.01 μmol, 58.85% yield) as off-white solid. LCMS m/z 1028.3 (M+H)+. 1H-NMR (400 MHz, DMSO-d₆): δ 10.58 (s, 1H), 8.84 (dd, J=8.40, 30.00 Hz, 2H), 8.24-8.22 (m, 1H), 8.13-8.10 (m, 1H), 7.85-7.80 (m, 2H), 7.61-7.49 (m, 4H), 7.27 (d, J=3.60 Hz, 1H), 7.10 (d, J=8.40 Hz, 1H), 6.14 (s, 1H), 4.86 (d, J=18.00 Hz, 1H), 4.40-4.38 (m, 1H), 4.28 (d, J=18.40 Hz, 1H), 4.10-4.07 (m, 2H), 3.98-3.91 (m, 7H), 3.47 (d, J=13.20 Hz, 2H), 3.28-3.12 (m, 3H), 3.02-2.99 (m, 1H), 2.78-2.73 (m, 4H), 2.68-2.65 (m, 2H), 2.51-2.50 (m, 2H), 2.36-2.18 (m, 1H), 1.92-1.45 (m, 6H).

### Example 51. 2-(6,7-dihydro-5H-pyrrolo[1,2-c]imidazol-1-yl)-2-[6-[4-[2-[2-[1-[4-[[(3R)-2,6-dioxo-3-piperidyl]-amino]-2-fluoro-phenyl]-4-hydroxy-4-piperidyl]acetyl]-2,6-diazaspiro[3.3]heptan-6-yl]phenyl]-4-fluoro-1-oxo-isoindolin-2-yl]-N-thiazol-2-yl-acetamide, (Compound 98)

### Step 1: 2-[1-[4-[[(3R)-2,6-dioxo-3-piperidyl]amino]-2-fluoro-phenyl]-4-hydroxy-4-piperidyl]acetic acid

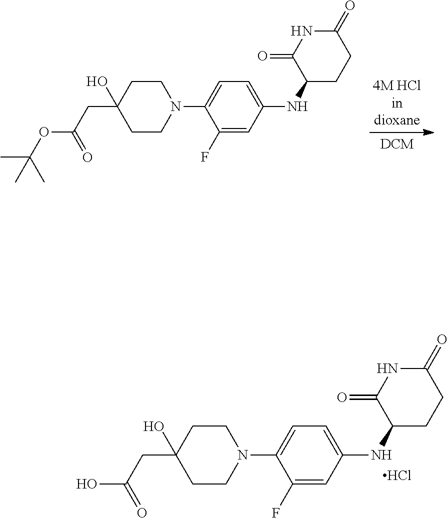 [SMILES: CC(C)(C)OC(=O)CC1(O)CCN(c2ccc(N[C@@H]3CCC(=O)NC3=O)cc2F)CC1 ; O=C(O)CC1(O)CCN(c2ccc(N[C@@H]3CCC(=O)NC3=O)cc2F)CC1 ; Cl]

To a stirred solution of tert-butyl 2-[1-[4-[[(3R)-2,6-dioxo-3-piperidyl]amino]-2-fluoro-phenyl]-4-hydroxy-4-piperidyl]acetate (530 mg, 1.22 mmol) in dichloromethane (10 mL) at 0° C. was added hydrogen chloride (4M in 1,4-dioxane, 1.52 mL, 6.09 mmol) dropwise. After addition allow reaction to stirred at room temperature for 6h. Volatiles were removed by rotary evaporation. The obtained residue was triturated with Diethyl ether (2×10 mL) two times. The solid residue was dried under rotary vacuum to afford 2-[1-[4-[[(3R)-2,6-dioxo-3-piperidyl]amino]-2-fluoro-phenyl]-4-hydroxy-4-piperidyl]acetic acid (470 mg, 928.08 μmol, 76.26% yield) as a bluish gray solid. LCMS (ESI+) m/z: 380.0 [M+H]⁺. ¹H-NMR (400 MHz, DMSO-d₆): δ 12.01 (bs, 1H), 10.86 (s, 1H), 7.64 (s, 1H), 6.70 (d, J=15.60 Hz, 1H), 6.58 (d, J=8.80 Hz, 1H), 4.43 (dd, J=12.00, 4.40 Hz, 2H), 3.79-3.61 (m, 2H), 3.41-3.38 (m, 2H), 2.74-2.68 (m, 1H), 2.61-2.53 (m, 1H), 2.46 (s, 3H), 2.30-2.19 (m, 2H), 2.09-2.06 (m, 1H), 1.94-1.88 (m, 3H).

### Step 2: 2-(6,7-dihydro-5H-pyrrolo[1,2-c]imidazol-1-yl)-2-[6-[4-[2-[2-[1-[4-[[(3R)-2,6-dioxo-3-piperidyl]amino]-2-fluoro-phenyl]-4-hydroxy-4-piperidyl]acetyl]-2,6-diazaspiro[3.3]heptan-6-yl]phenyl]-4-fluoro-1-oxo-isoindolin-2-yl]-N-thiazol-2-yl-acetamide

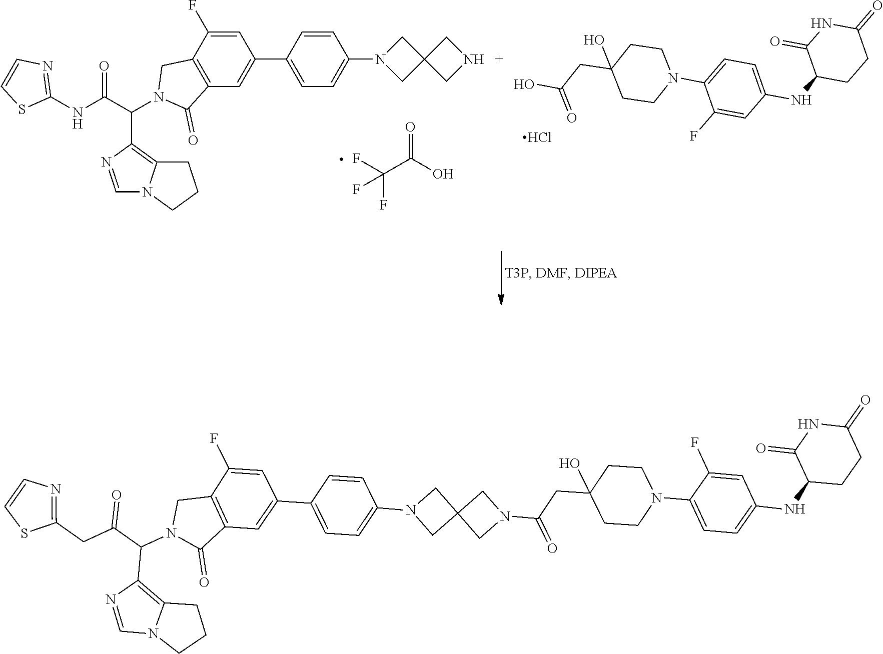

2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide, 2,2,2-trifluoroacetic acid (220 mg, 321.79 μmol), and 2-[1-[4-[[(3R)-2,6-dioxo-3-piperidyl]amino]-2-fluoro-phenyl]-4-hydroxy-4-piperidyl]acetic acid; hydrochloride (160.58 mg, 386.15 μmol) were mixed in NA-dimethylformamide (4 mL). N, N-diisopropylethylamine (207.95 mg, 1.61 mmol, 280.25 μL) was added to the reaction mixture at 0° C. Propyl phosphonic anhydride solution (50 wt. % in ethyl acetate, 204.77 mg, 643.58 μmol) was added to the reaction mixture at 0° C. The reaction mixture was stirred at ambient temperature for 2 h. The crude mixture was directly injected on a C18 column (100 g) for purification while eluting (0% to 50% of acetonitrile+0.1% ammonium acetate in water over 30 minutes, then steep gradient to 100% acetonitrile). The pure fraction was frozen and lyophilized to afford Compound 98 (88.39 mg, 93.64 μmol, 29.10% yield) as an off-white solid compound. LCMS (ESI+): 931.3 [M+H]. ¹H-NMR (400 MHz, DMSO-d₆): ¹H-NMR (400 MHz, DMSO-d₆): δ 12.58 (s, 1H), 10.78 (s, 1H), 7.75-7.70 (m, 2H), 7.65 (d, J=8.80 Hz, 2H), 7.61 (s, 1H), 7.49 (d, J=3.60 Hz, 1H), 7.26 (d, J=3.60 Hz, 1H), 6.86 (t, J=9.60 Hz, 1H), 6.55 (d, J=8.40 Hz, 2H), 6.50 (dd, J=2.40, 14.80 Hz, 1H), 6.42 (dd, J=2.40, 8.80 Hz, 1H), 6.15 (s, 1H), 5.78 (d, J=7.60 Hz, 1H), 4.82-4.76 (m, 2H), 4.39 (bs, 2H), 4.26-4.20 (m, 2H), 4.09 (bs, 2H), 4.05-3.96 (m, 6H), 2.90-2.82 (m, 4H), 2.77-2.68 (m, 3H), 2.23 (bs, 2H), 2.14-2.01 (m, 1H), 1.92-1.81 (m, 1H), 1.80-1.72 (m, 3H), 1.67-1.56 (m, 2H). (Proton signals were not observed due to water obscuration)

### Example 52. 2-(6,7-dihydro-5H-pyrrolo[1,2-c]imidazol-1-yl)-2-[6-[4-[2-[2-[1-[4-[(2,6-dioxo-3-piperidyl)amino]-2-fluoro-phenyl]-4-hydroxy-4-piperidyl]acetyl]-2,6-diazaspiro[3.3]heptan-6-yl]phenyl]-4-fluoro-1-oxo-isoindolin-2-yl]-N-(2-pyridyl)acetamide, (Compound 99)

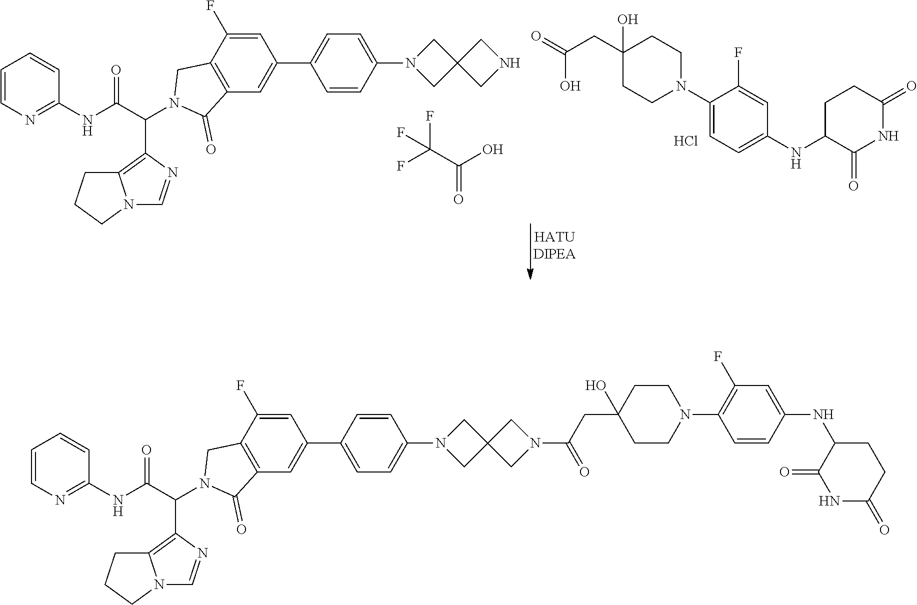

2-[1-[4-[(2,6-dioxo-3-piperidyl)amino]-2-fluoro-phenyl]-4-hydroxy-4-piperidyl]acetic acid hydrochloride (110 mg, 264.52 μmol) and 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-(2-pyridyl)acetamide, trifluoroacetic acid salt (179.25 mg, 264.52 μmol) were mixed in N,N-dimethylformamide, the reaction mixture was cooled to 0° C. N,N-diisopropylethylamine (170.93 mg, 1.32 mmol, 230.37 μL) was added to the reaction mixture, and 1-[bis(dimethylamino)methylene]-1H-1,2,3-triazolo[4,5-b]pyridinium 3-oxide hexafluorophosphate (110.64 mg, 290.98 μmol) was added, and the reaction mixture was stirred for 4 h. The mixture was injected on a 50 g C18 column and purified using a 0% to 100% acetonitrile in water (+0.1% trifluoroacetic acid) water elution gradient. The pure fractions were pooled, partitioned between ethyl acetate and sodium bicarbonate (aq., sat.). The organic layer was washed with brine, dried with sodium sulfate, filtered, and evaporated under reduced pressure. The residue was purified by silica gel chromatography (24 g column, 0% to 20% methanol in ethyl acetate). Pure fractions were evaporated under reduced pressure to afford Compound 99 (80 mg, 83.89 μmol, 31.71% yield). LCMS (ESI+): 925.3 (M+H)/463.4 (M/2+H). ¹H NMR (400 MHz, DMSO-d₆) δ 10.83 (s, 1H), 10.71 (s, 1H), 8.25 (ddd, J=4.9, 2.0, 0.9 Hz, 1H), 8.00 (d, J=8.4 Hz, 1H), 7.72 (ddd, J=8.7, 7.4, 2.0 Hz, 1H), 7.67 (s, 1H), 7.62 (d, J=10.7 Hz, 1H), 7.60-7.42 (m, 3H), 7.05 (ddd, J=7.4, 4.9, 1.1 Hz, 1H), 6.78 (dd, J=10.0, 8.7 Hz, 1H), 6.47 (d, J=8.5 Hz, 2H), 6.43 (dd, J=15.1, 2.6 Hz, 1H), 6.34 (dd, J=8.7, 2.6 Hz, 1H), 6.13 (s, 1H), 5.70 (d, J=7.6 Hz, 1H), 4.72 (d, J=17.7 Hz, 1H), 4.69 (s, 1H), 4.31 (s, 2H), 4.23-4.09 (m, 2H), 4.01 (s, 2H), 3.99-3.84 (m, 6H), 2.96-2.57 (m, 4H), 2.57-2.45 (m, 2H), 2.15 (s, 2H), 2.08-1.91 (m, 1H), 1.92-1.64 (m, 3H), 1.55 (d, J=12.4 Hz, 2H).

### Example 53. 2-(6,7-dihydro-5H-pyrrolo[1,2-c]imidazol-1-yl)-2-[6-[4-[2-[2-[1-[4-[(2,6-dioxo-3-piperidyl)-amino]-2,6-difluoro-phenyl]-4-hydroxy-4-piperidyl]acetyl]-2,6-diazaspiro[3.3]hept-an-6-yl]-phenyl]-4-fluoro-1-oxo-isoindolin-2-yl]-N-thiazol-2-yl-acetamide, (Compound 103)

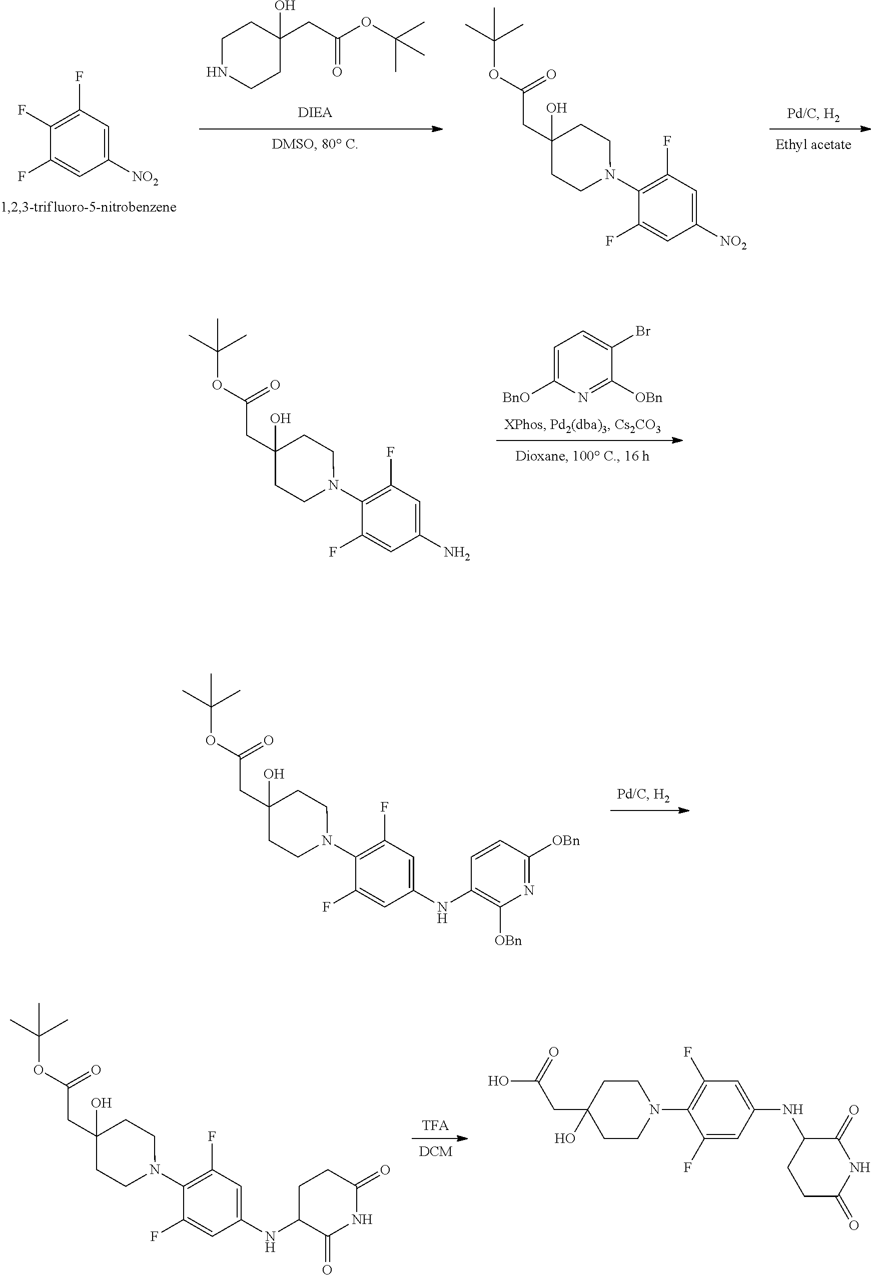

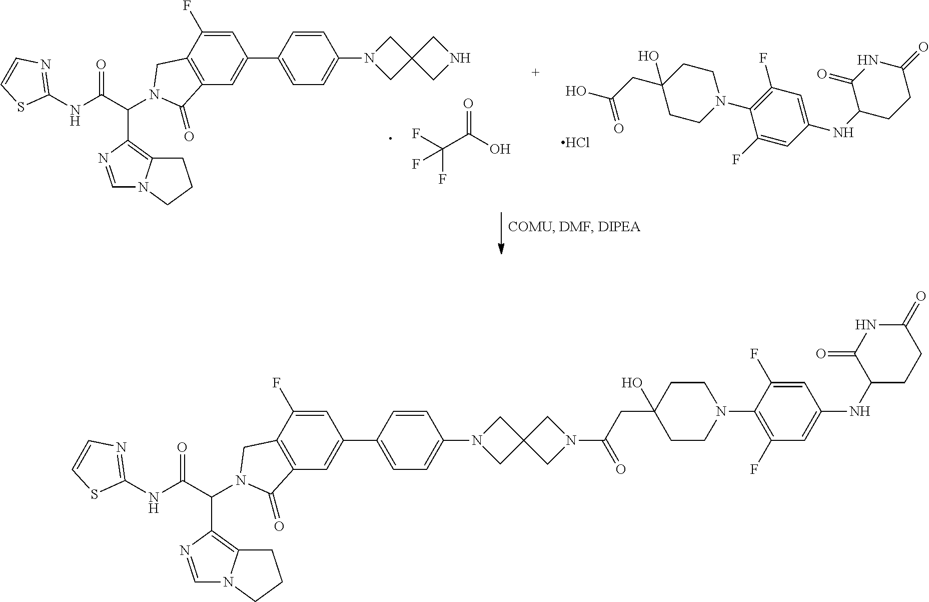 [SMILES: O=C(Nc1nccs1)C(c1ncn2c1CCC2)N1Cc2c(F)cc(-c3ccc(N4CC5(CNC5)C4)cc3)cc2C1=O ; O=C(O)C(F)(F)F ; Cl ; O=C(O)CC1(O)CCN(c2c(F)cc(NC3CCC(=O)NC3=O)cc2F)CC1 ; "COMU, DMF, DIPEA" ; O=C1CCC(Nc2cc(F)c(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1]

### Step 1: tert-Butyl 2-[1-(2,6-difluoro-4-nitro-phenyl)-4-hydroxy-4-piperidyl]acetate

To a solution of tert-butyl 2-(4-hydroxy-4-piperidyl) acetate (6.69 g, 31.06 mmol) and 1,2,3-trifluoro-5-nitro-benzene (5 g, 28.24 mmol) in DMSO (50 mL) was added N,N-diisopropylethylamine (10.95 g, 84.71 mmol, 14.75 mL). The mixture was stirred at 80° C. for 1 h. The mixture was poured into water (200 mL). The precipitated solid was collected by filtering and dried under high vacuum without purification to afford tert-butyl 2-[1-(2,6-difluoro-4-nitro-phenyl)-4-hydroxy-4-piperidyl]acetate (10.5 g, 24.81 mmol, 87.88% yield) as a yellow oil. ¹H NMR (400 MHz, DMSO-d₆) δ=7.95 (d, J=10.4 Hz, 2H), 4.66 (s, 1H), 3.47-3.39 (m, 2H), 3.25 (d, J=12.8 Hz, 2H), 2.37 (s, 2H), 1.82-1.72 (m, 2H), 1.69-1.62 (m, 2H), 1.41-1.40 (s, 9H).

### Step 2: tert-Butyl 2-(1-(4-amino-2,6-difluorophenyl)-4-hydroxypiperidin-4-yl) acetate

To a solution of tert-butyl 2-[1-(2,6-difluoro-4-nitro-phenyl)-4-hydroxy-4-piperidyl]acetate (10 g, 26.86 mmol) in ethyl acetate (100 mL) was added palladium, 10% on charcoal (6.00 g, 4.94 mmol) under N₂ atmosphere. The suspension was degassed and purged with H₂ for 3 times. The mixture was stirred under H₂ (15 Psi) at 25° C. for 6 h. The reaction mixture was filtered, the filtrate was concentrated in vacuum to give tert-butyl 2-(1-(4-amino-2,6-difluorophenyl)-4-hydroxypiperidin-4-yl) acetate (9.1 g, 25.24 mmol, 94.02% yield) as yellow oil. ¹H NMR (400 MHz, DMSO-d₆) δ=6.16-6.06 (m, 2H), 5.38 (s, 2H), 4.40 (s, 1H), 3.17 (d, J=5.2 Hz, 2H), 2.70-2.62 (m, 2H), 2.32 (s, 2H), 1.74-1.65 (m, 2H), 1.58 (br d, J=12.4 Hz, 2H), 1.41 (s, 9H).

### Step 3: tert-Butyl 2-(1-(4-((2,6-bis(benzyloxy)pyridin-3-yl)amino)-2,6-difluorophenyl)-4-hydroxypiperidin-4-yl)acetate

A stirred solution of tert-butyl 2-[1-(4-amino-2,6-difluoro-phenyl)-4-hydroxy-4-piperidyl]acetate (8.7 g, 25.41 mmol), 2,6-dibenzyloxy-3-bromo-pyridine (12.47 g, 33.68 mmol) and CS₂CO₃ (27.41 g, 76.23 mmol) in 1,4-dioxane (360 mL), the reaction mixture was degassed with nitrogen for 15 minutes. Xphos (1.34 g, 2.54 mmol) and Pd₂(dba)₃ (2.57 g, 2.54 mmol) were added and the mixture was degassed with nitrogen for 5 minutes. The reaction mixture was heated to 100° C. for 16h. The reaction mixture was concentrated, diluted with water. The mixture was extracted with ethyl acetate (200 mL×3). The combined organic phase was washed with brine (200×2 mL), dried with anhydrous sodium sulfate, filtered, and concentrated in vacuum to give a residue. The residue was purified by silica gel chromatography (Petroleum ether/Ethyl acetate=5:1) to afford tert-butyl 2-[1-[4-[(2,6-dibenzyloxy-3-pyridyl)amino]-2,6-difluoro-phenyl]-4-hydroxy-4-piperidyl]acetate (12.75 g, 19.17 mmol, 75.5% yield) as a yellow oil. LCMS (ESI): m/z 632.2 [M+H]⁺, ¹H NMR (400 MHz, DMSO-d₆) δ=7.61 (s, 1H), 7.54 (d, J=8.4 Hz, 1H), 7.43-7.27 (m, 10H), 6.44 (d, J=8.4 Hz, 1H), 6.28-6.21 (m, 2H), 5.38 (s, 2H), 5.30 (s, 2H), 4.44 (s, 1H), 3.23 (t, J=10.4 Hz, 2H), 2.76-2.68 (m, 2H), 2.33 (s, 2H), 1.77-1.67 (m, 2H), 1.64-1.57 (m, 2H), 1.41 (s, 9H).

### Step 4: tert-Butyl 2-(1-(4-((2,6-dioxopiperidin-3-yl)amino)-2,6-difluorophenyl)-4-hydroxypiperidin-4-yl)acetate

To a solution of tert-butyl 2-[1-[4-[(2,6-dibenzyloxy-3-pyridyl)amino]-2,6-difluoro-phenyl]-4-hydroxy-4-piperidyl]acetate (12.75 g, 20.18 mmol) in 1,4-dioxane (850 mL) was added Pd/C (8.29 g, 68.24 mmol) under N₂ atmosphere. The suspension was degassed and purged with H₂ for 3 times. The mixture was stirred under H₂ (15 Psi) at 25° C. for 16 h. The reaction mixture was filtered, the mother solution concentrated in vacuum to afford tert-butyl 2-(1-(4-((2,6-dioxopiperidin-3-yl)amino)-2,6-difluorophenyl)-4-hydroxypiperidin-4-yl)acetate (8.5 g, 16.49 mmol, 81.72% yield). LCMS (ESI): m/z 454.1 [M+H]⁺, ¹H NMR (400 MHz, DMSO-d₆) δ=10.80 (s, 1H), 6.30 (d, J=12.0 Hz, 2H), 6.20 (d, J=8.0 Hz, 1H), 4.44-4.39 (m, 1H), 4.35-4.26 (m, 1H), 3.26-3.19 (m, 2H), 2.74-2.66 (m, 1H), 2.71-2.65 (m, 1H), 2.61-2.56 (m, 1H), 2.33 (s, 2H), 2.11-2.01 (m, 1H), 1.90-1.79 (m, 1H), 1.75-1.66 (m, 2H), 1.63-1.56 (m, 2H), 1.41 (s, 9H)

### Step 5: 2-[1-[4-[(2,6-dioxo-3-piperidyl)amino]-2,6-difluoro-phenyl]-4-hydroxy-4-piperidyl]acetic acid hydrochloride

To a stirred solution of tert-butyl 2-[1-[4-[(2,6-dioxo-3-piperidyl)amino]-2,6-difluoro-phenyl]-4-hydroxy-4-piperidyl]acetate (200 mg, 441.04 μmol) in dichloromethane (2 mL) was cooled at 0° C. and added hydrogen chloride (4M in 1,4-dioxane, 1.1 mL, 4.41 mmol) drop wise into reaction mixture. After addition allow the reaction mixture stirred at room temperature for 16 h. The reaction mixture was evaporated under reduced pressure. The obtained solid was washed by diethyl ether (30 mL) to afford crude solid 2-[1-[4-[(2,6-dioxo-3-piperidyl)amino]-2,6-difluoro-phenyl]-4-hydroxy-4-piperidyl]acetic acid hydrochloride (182 mg, 394.97 μmol, 89.56% yield). This crude compound was carried forward next step without purification. LCMS m/z: 398.1 [M+1]

### Step 6: 2-(6,7-dihydro-5H-pyrrolo[1,2-c]imidazol-1-yl)-2-[6-[4-[2-[2-[1-[4-[(2,6-dioxo-3-piper-idyl)amino]-2,6-difluoro-phenyl]-4-hydroxy-4-piperidyl]acetyl]-2,6-diazaspiro[3.3]heptan-6-yl]-phenyl]-4-fluoro-1-oxo-isoindolin-2-yl]-N-thiazol-2-yl-acetamide

To a solution of 2-[1-[4-[(2,6-dioxo-3-piperidyl)amino]-2,6-difluoro-phenyl]-4-piperidyl]acetic acid hydrochloride (98.81 mg, 227.76 μmol) in N,N-dimethylformamide (2 mL) was added N,N-diisopropylethylamine (128.55 mg, 994.63 μmol, 173.25 μL) at 0° C. 1-[(1-(Cyano-2-ethoxy-2-oxoethylideneaminooxy)-dimethylamino-morpholino)] uronium hexafluorophosphate (159.74 mg, 372.98 μmol) was added to the reaction mixture at 0° C. After 10 minutes, 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide, trifluoroacetic acid salt (170 mg, 248.66 μmol), was added to the reaction mixture at the same temperature. The reaction mixture was stirred at ambient temperature for 2 h. The crude mixture was directly injected on a C18 column (60 g) for purification while eluting (0% to 70% of acetonitrile in water (+0.1% ammonium acetate) over 30 minutes, then steep gradient to 100% acetonitrile). The pure fraction was frozen and lyophilized to afford Compound 103 (25.05 mg, 25.48 μmol, 10.25% yield) as an off white solid compound. LCMS (ESI+): 949.3 [M+H]. ¹H-NMR (400 MHz, DMSO-d₆): δ 12.53 (s, 1H), 10.82 (s, 1H), 7.75 (s, 1H), 7.71 (d, J=10.80 Hz, 1H), 7.65 (d, J=8.40 Hz, 2H), 7.61 (s, 1H), 7.49 (d, J=3.60 Hz, 1H), 7.27 (d, J=3.60 Hz, 1H), 6.55 (d, J=8.80 Hz, 2H), 6.32 (d, J=12.40 Hz, 2H), 6.32 (d, J=12.40 Hz, 1H), 6.15 (s, 1H), 4.80 (d, J=17.60 Hz, 1H), 4.75 (s, 1H), 4.39 (s, 2H), 4.38-4.27 (m, 1H), 4.22 (d, J=17.60 Hz, 1H), 4.09 (s, 2H), 4.03-3.96 (m, 7H), 3.30-3.21 (m, 3H), 2.78-2.67 (m, 5H), 2.22 (s, 2H), 2.12-2.02 (m, 1H), 1.89-1.79 (m, 1H), 1.71-1.69 (m, 2H), 1.59-1.56 (m, 2H).

### Example 54. 2-(6,7-dihydro-5H-pyrrolo[1,2-c]imidazol-1-yl)-2-[6-[4-[2-[2-[1-[4-[[2,6-dioxo-3-piperidyl]amino]-2-fluoro-5-methoxy-phenyl]-4-hydroxy-4-piperidyl]acetyl]-2,6-diazaspiro[3.3]heptan-6-yl]phenyl]-4-fluoro-1-oxo-isoindolin-2-yl]-N-thiazol-2-yl-acetamide, isomer 1, (Compound 173)

### Step 1: 1,2-difluoro-4-methoxy-5-nitro-benzene

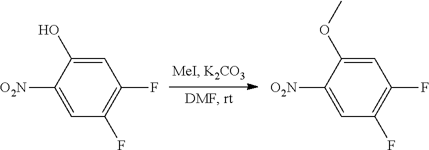

To a solution of 4,5-difluoro-2-nitro-phenol (500 mg, 2.86 mmol) in N,N-dimethylformamide (5 mL) were added potassium carbonate (1.18 g, 8.57 mmol, 517.04 μL) and iodomethane (1.22 g, 8.57 mmol, 533.33 μL). The mixture was stirred at 25° C. for 3 h. Ethyl acetate (20 mL) and water (20 mL) were added to the reaction mixture and layers were separated. The organic phase was washed with water (3×20 mL) followed by brine (3×20 mL), dried over anhydrous sodium sulfate, filtered and concentrated under reduced pressure to give 1,2-difluoro-4-methoxy-5-nitro-benzene (550 mg, 2.91 mmol) as a yellow solid. ¹H NMR (400 MHz, DMSO-d₆) δ=8.23 (dd, J=8.4, 10.0 Hz, 1H), 7.62 (dd, J=6.8, 12.4 Hz, 1H), 3.93 (s, 3H).

### Step 2: tert-butyl 2-[1-(2-fluoro-5-methoxy-4-nitro-phenyl)-4-hydroxy-4-piperidyl]acetate

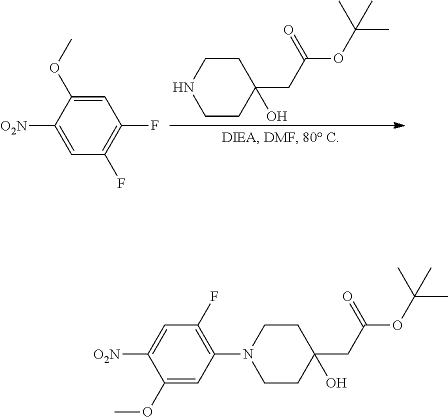 [SMILES: COc1cc(F)c(F)cc1[N+](=O)[O-] ; CC(C)(C)OC(=O)CC1(O)CCNCC1 ; COc1cc(N2CCC(O)(CC(=O)OC(C)(C)C)CC2)c(F)cc1[N+](=O)[O-]]

To a solution of 1,2-difluoro-4-methoxy-5-nitro-benzene (550 mg, 2.91 mmol) in N,N-dimethylformamide (5 mL) were added tert-butyl 2-(4-hydroxy-4-piperidyl)acetate (626 mg, 2.91 mmol) and N-ethyl-N-isopropylpropan-2-amine (645.54 mg, 4.99 mmol, 0.87 mL). The mixture was stirred at 80° C. for 12 h. After being cooled to 25° C., ethyl acetate (20 mL) and water (20 mL) were added to the reaction mixture and layers were separated. The organic phase was washed with water (3×20 mL) followed by brine (3×20 mL), dried over anhydrous sodium sulfate, filtered and concentrated under reduced pressure to give tert-butyl 2-[1-(2-fluoro-5-methoxy-4-nitro-phenyl)-4-hydroxy-4-piperidyl]acetate (1 g, 2.47 mmol, 85% yield) as a yellow solid. LCMS (ESI): m/z 385.1 [M+H]⁺

### Step 3: tert-butyl 2-[1-(4-amino-2-fluoro-5-methoxy-phenyl)-4-hydroxy-4-piperidyl]acetate

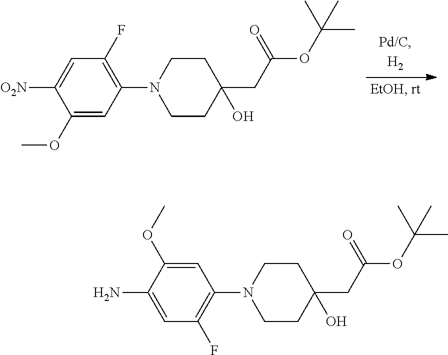 [SMILES: COc1cc(N2CCC(O)(CC(=O)OC(C)(C)C)CC2)c(F)cc1[N+](=O)[O-] ; COc1cc(N2CCC(O)(CC(=O)OC(C)(C)C)CC2)c(F)cc1N]

To a solution of tert-butyl 2-[1-(2-fluoro-5-methoxy-4-nitro-phenyl)-4-hydroxy-4-piperidyl]acetate (1 g, 2.60 mmol) in ethanol (10 mL) was added palladium, 10% in carbon (100 mg) under nitrogen. The mixture was stirred at 25° C. under hydrogen (15 psi) for 12 h. The reaction mixture was filtered through a pad of Celite and the filter cake was washed with ethanol (50 mL). The filtrate was concentrated under vacuum to give tert-butyl 2-[1-(4-amino-2-fluoro-5-methoxy-phenyl)-4-hydroxy-4-piperidyl]acetate (1 g, 2.60 mmol, >98% yield) as a brown solid. LCMS (ESI): m/z 355.1 [M+H]⁺

### Step 4: tert-butyl 2-[1-[4-[(2,6-dioxo-3-piperidyl)amino]-2-fluoro-5-methoxy-phenyl]-4-hydroxy-4-piperidyl]acetate

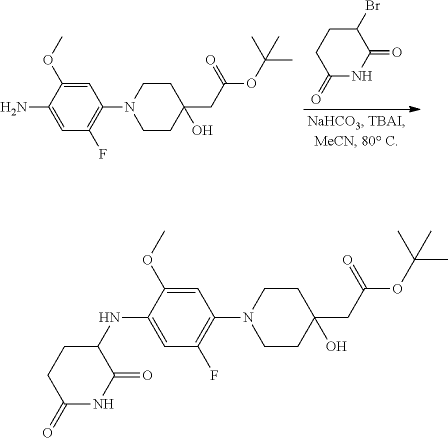

To a solution of tert-butyl 2-[1-(4-amino-2-fluoro-5-methoxy-phenyl)-4-hydroxy-4-piperidyl]acetate (200 mg, 564.31 μmol) and 3-bromopiperidine-2,6-dione (217 mg, 1.13 mmol) in acetonitrile (2 mL) were added sodium bicarbonate (142 mg, 1.69 mmol, 65.74 μL) and tetrabutylammonium iodide (21 mg, 56.85 μmol). The mixture was stirred at 80° C. for 16 h. After being cooled to 25° C., the reaction mixture was concentrated under reduce pressure. The residue was purified by column chromatography on silica gel (petroleum ether/ethyl acetate=5/1 to 1/4) to give tert-butyl 2-[1-[4-[(2,6-dioxo-3-piperidyl)amino]-2-fluoro-5-methoxy-phenyl]-4-hydroxy-4-piperidyl]acetate (200 mg, 421.04 μmol, 75% yield) as a brown solid. LCMS (ESI): m/z 466.2 [M+H]⁺

### Step 5: tert-butyl 2-[1-[4-[[2,6-dioxo-3-piperidyl]amino]-2-fluoro-5-methoxy-phenyl]-4-hydroxy-4-piperidyl]acetate, isomer 1 and tert-butyl 2-[1-[4-[[2,6-dioxo-3-piperidyl]amino]-2-fluoro-5-methoxy-phenyl]-4-hydroxy-4-piperidyl]acetate, isomer 2

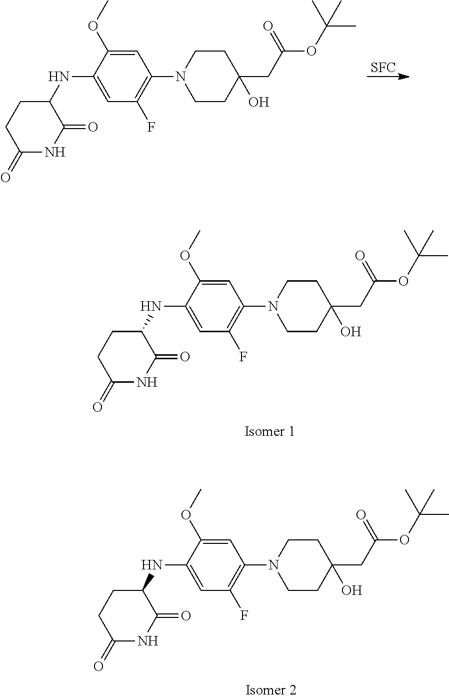

Racemic tert-butyl 2-[1-[4-[(2,6-dioxo-3-piperidyl)amino]-2-fluoro-5-methoxy-phenyl]-4-hydroxy-4-piperidyl]acetate was separated by Chiral SFC (Isopropanol condition, column: Phenomenex-Cellulose-2 (250 mm×30 mm, 10 μm); B %: 50%-50%; 4.0 min, 25 min) to afford two sets of fractions. The first eluting set of fractions was evaporated to afford tert-butyl 2-[1-[4-[[2,6-dioxo-3-piperidyl]amino]-2-fluoro-5-methoxy-phenyl]-4-hydroxy-4-piperidyl]acetate, isomer 1 (100 mg, 212.67 μmol, 50% yield) as a brown solid (LCMS (ESI): m/z 466.1 [M+H]⁺). The second set of fractions was evaporated to afford tert-butyl 2-[1-[4-[[(3R)-2,6-dioxo-3-piperidyl]amino]-2-fluoro-5-methoxy-phenyl]-4-hydroxy-4-piperidyl]acetate (70 mg, 148.87 μmol, 35% yield) as a brown solid (LCMS (ESI): m/z 466.1 [M+H]⁺).

### Step 6: 2-[1-[4-[[2,6-dioxo-3-piperidyl]amino]-2-fluoro-5-methoxy-phenyl]-4-hydroxy-4-piperidyl]acetic acid, Isomer 1

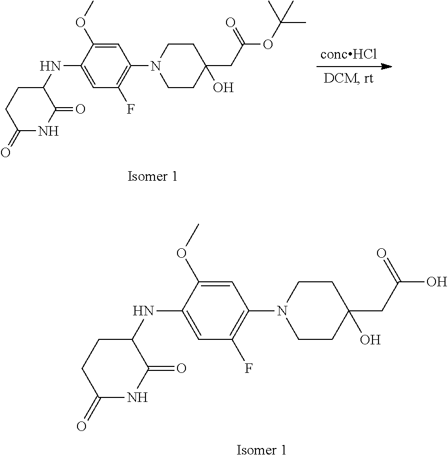 [SMILES: COc1cc(N2CCC(O)(CC(=O)OC(C)(C)C)CC2)c(F)cc1NC1CCC(=O)NC1=O ; COc1cc(N2CCC(O)(CC(=O)O)CC2)c(F)cc1NC1CCC(=O)NC1=O]

To a solution of tert-butyl 2-[1-[4-[[2,6-dioxo-3-piperidyl]amino]-2-fluoro-5-methoxy-phenyl]-4-hydroxy-4-piperidyl]acetate, isomer 1 (100 mg, 214.82 μmol) in dichloromethane (1.5 mL) was added hydrochloric acid (12 M, 0.1 mL). The mixture was stirred at 25° C. for 0.5 h. The mixture was concentrated under reduced pressure at 35° C. to give 2-[1-[4-[[2,6-dioxo-3-piperidyl]amino]-2-fluoro-5-methoxy-phenyl]-4-hydroxy-4-piperidyl]acetic acid, hydrochloride (92 mg, 196.02 μmol, 91% yield, HCl salt) as a brown oil. LCMS (ESI): m/z 410.1 [M+H]+

### Step 7: 2-(6,7-dihydro-5H-pyrrolo[1,2-c]imidazol-1-yl)-2-[6-[4-[2-[2-[1-[4-[[(3S)-2,6-dioxo-3-piperidyl]amino]-2-fluoro-5-methoxy-phenyl]-4-hydroxy-4-piperidyl]acetyl]-2,6-diazaspiro[3.3]heptan-6-yl]phenyl]-4-fluoro-1-oxo-isoindolin-2-yl]-N-thiazol-2-yl-acetamide, Isomer 1

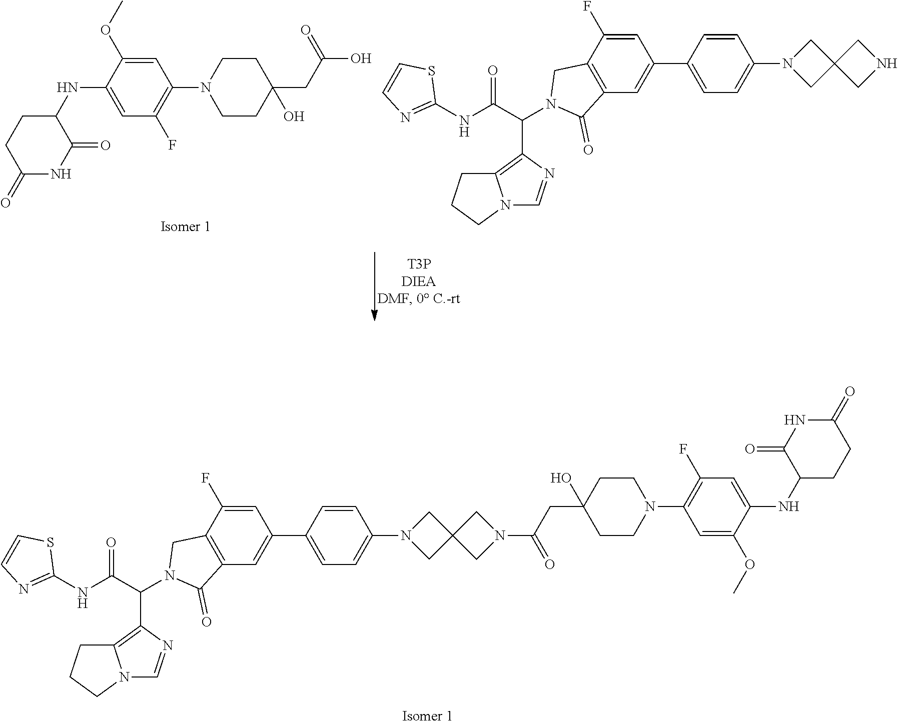

To a solution of 2-[1-[4-[[2,6-dioxo-3-piperidyl]amino]-2-fluoro-5-methoxy-phenyl]-4-hydroxy-4-piperidyl]acetic acid, isomer 1, hydrochloride (92 mg, 206.34 μmol) in N,N-dimethylformamide (2 mL) were added N-ethyl-N-isopropylpropan-2-amine (192.92 mg, 1.49 mmol, 260.00 μL) and propylphosphonic anhydride, 50% in ethyl acetate (132 mg, 207.43 μmol) at 0° C. The mixture was stirred at 0° C. for 20 min. Then 2-[6-[4-(2,6-diazaspiro[3.3]heptan-2-yl)phenyl]-4-fluoro-1-oxo-isoindolin-2-yl]-2-(6,7-dihydro-5H-pyrrolo[1,2-c]imidazol-1-yl)-N-thiazol-2-yl-acetamide, trifluoroacetic acid salt (113 mg, 165.28 μmol) was added and the mixture was stirred at 25° C. for 12 h. The reaction mixture was filtered. The filtrate was purified by reversed phase column (flow: 40 mL/min; gradient: from 5-51% acetonitrile in water over 28 min; column: I.D. 31 mm×H 140 mm, Welch Ultimate Xb C18 20-40 μm; 120 Å) and lyophilized to give Compound 173 (51.97 mg, 53.54 μmol, 26% yield) as a white solid. LCMS (ESI): m/z 961.3 [M+H]⁺, ¹H NMR (400 MHz, DMSO-d₆) δ=12.50 (s, 1H), 10.84 (s, 1H), 7.74 (s, 1H), 7.70 (d, J=10.8 Hz, 1H), 7.64 (d, J=8.8 Hz, 2H), 7.60 (s, 1H), 7.48 (d, J=3.6 Hz, 1H), 7.25 (d, J=3.6 Hz, 1H), 6.62 (d, J=8.0 Hz, 1H), 6.58-6.50 (m, 3H), 6.14 (s, 1H), 5.07 (d, J=6.8 Hz, 1H), 4.83-4.74 (m, 2H), 4.38 (s, 2H), 4.28-4.18 (m, 2H), 4.08 (s, 2H), 4.04-3.92 (m, 6H), 3.79 (s, 3H), 3.23 (d, J=6.4 Hz, 2H), 3.00-2.91 (m, 2H), 2.91-2.82 (m, 2H), 2.82-2.82 (m, 1H), 2.82-2.72 (m, 2H), 2.56 (s, 1H), 2.43 (s, 1H), 2.23 (s, 2H), 2.17-2.08 (m, 1H), 1.98-1.84 (m, 1H), 1.82-1.72 (m, 2H), 1.63 (d, J=12.4 Hz, 2H).

### Example 55: Efficacy of Compound 1 in Non-Small Cell Lung Cancer

Compound 1 is a potent and selective degrader of EGFR L858R that is active in vitro and in vivo. Compound 1 is active, as a single agent, in models of EGFR L858R driven NSCLC without resistance-causing secondary mutations in EGFR, and in similar models that do harbor secondary resistance mutations (such as T790M and C797S). Compound 1 may be active in EGFR L858R driven NSCLC in the front-line setting and avoid the emergence of resistance-causing secondary EGFR mutations seen with EGFR inhibitors, as well as in the setting of resistance to EGFR inhibitors.

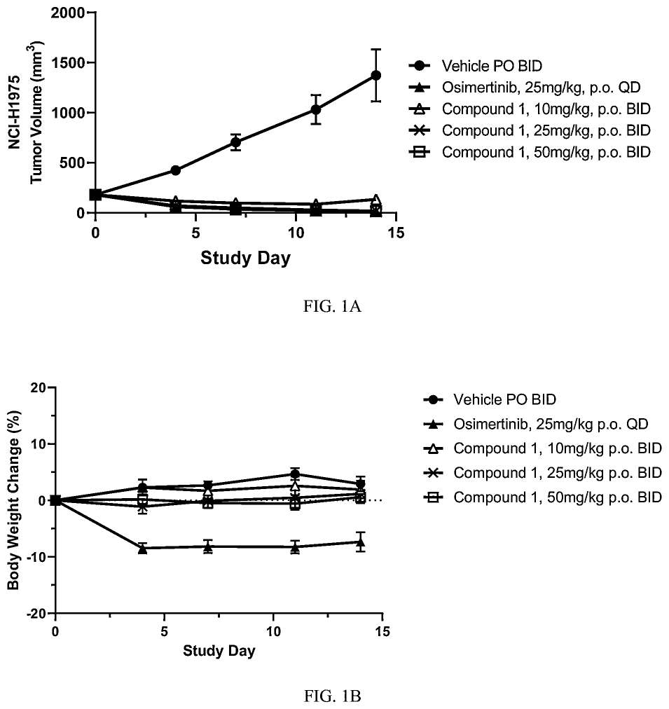

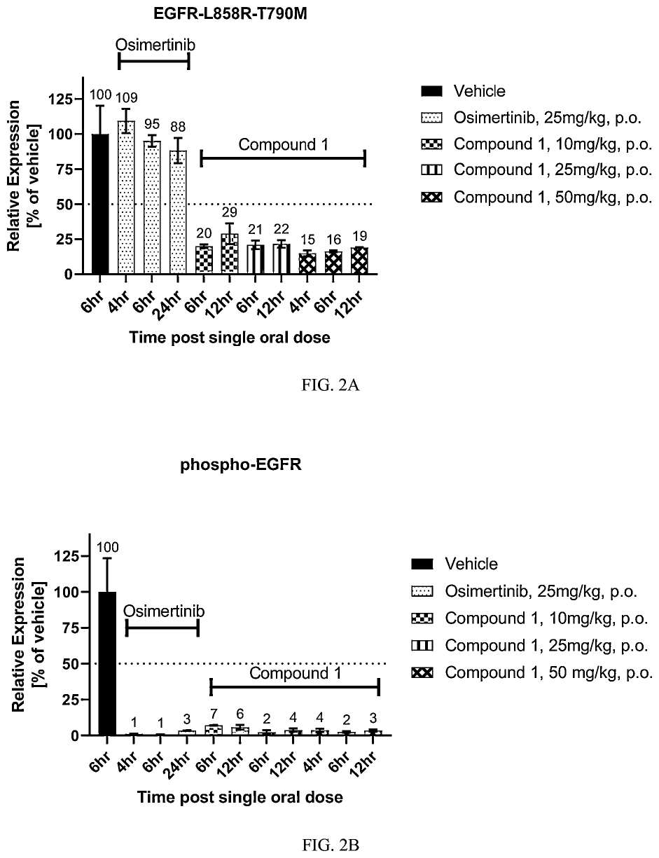

Compound 1 in vivo efficacy in a non-small cell lung cancer (NSCLC) cell line-derived xenograft (NCI-H1975 EGFR-L858R-T790M) was evaluated at three different concentrations: 20 mg/kg/day, 50 mg/kg/day, and 100 mg/kg/day. Compound 1 was administered BID (twice a day) orally (PO). The efficacy of Compound 1 was compared to osimertinib (administered at 25 mg/kg/day). The results in the NCI-H1975 are shown in FIG. 1. Table 11 describes the statistical significance of the experiment for each dose. Treatment with Compound 1 at all 20, 50 and 100 mg/kg resulted in a reduced tumor volume.

TABLE 11
Statistical Significance of Compound 1 Compared to Vehicle

| | | |
|---|---|---|
| | | Statistical Significance |
| | Dose Level | Compared to Vehicle |
| Treatment | (mg/kg/day) | (p-value) |

NCI-H1975 Xenograft

| | | |
|---|---|---|
| Vehicle | N/A | N/A |
| Compound 1 | 20 | <0.001 |
| Compound 1 | 50 | <0.001 |
| Compound 1 | 100 | <0.001 |
| Osimertinib | 25 | <0.001 |

For the NCI-H1975 cells, the xenograft study was conducted in female BALB/c nude mice bearing NCI-H1975 NSCLC tumors. Female nude mice were inoculated subcutaneously in the right flank with 5×10⁶ tumors cells in 0.1 mL of PBS. Tumor volume was measured twice weekly in two dimensions using calipers, and volume (mm³) was calculated using the formula: V=0.5 a×b² where a and b are the long and short diameters of the tumor in mm, respectively. Once the tumors reached an average tumor volume of 181 mm³ (13 days after implantation), the animals were divided randomly into groups of 6, stratified to result in about equal average tumor sizes in each treatment group. Treatment began on Day 0 with established subcutaneous NCI-H1975 tumors with a mean tumor volume (MTV) of 181 mm³.

All agents were administered to mice bearing NCI-H1975 tumors on day 0 and dosed PO daily (osimertinib) or twice a day (Compound 1) for 14 days. Compound 1 was dosed at 20, 50, or 100 mg/kg/day and was formulated in PEG400 (20% v/v)+80% v/v SBECD (25% w/v) in ddH2O. Osimertinib was dosed at 25 mg/kg/day and was formulated in 1% Tween 80. Body weight and MTV were measured on a 2× weekly schedule. Statistical analysis was performed using a one-way ANOVA. Data are expressed as MTV±SEM.

### Example 56. Compound 1 Demonstrates Potent EGFR-L858R Degradation and Phospho-EGFR Inhibition in Non-Small Lung Cancer in vivo Model

Compound 1 was administered as a single oral (PO) dose at 10, 25, or 50 mg/kg to female BALB/c nude mice with NCI-H1975 tumors. At 4h, 6h or 12h tumors were harvested and the protein expression of mutant EGFR-L858R and phospho-EGFR was measured by western blot. Compound 1 shows potent EGFR degradation and phospho-EGFR inhibition at 4, 6, and 12 hours.

A xenograft study was conducted in female BALB/c nude mice bearing NCI-H1975 NSCLC tumors. Female nude mice were inoculated subcutaneously in the right flank with 5×10⁶ tumors cells in 0.1 mL of PBS. Tumor volume was measured twice weekly in two dimensions using calipers, and volume (mm³) was calculated using the formula: V=0.5 a×b2 where a and b are the long and short diameters of the tumor in mm, respectively. Once the tumors reached an average volume of 363 mm³, the animals were divided randomly into groups of 3 for the single dose study.

All agents were administered to mice bearing NCI-H1975 tumors on day 0 as a single oral (PO) dose. Compound 1 was dosed at 10, 25, or 50 mg/kg and was formulated in PEG400 (20% v/v)+80% v/v SBECD (25% w/v) in ddH2O, which was also used as the vehicle control. Osimertinib was dosed at 25 mg/kg and was formulated in 1% Tween 80. For the vehicle group, 3 mice were sacrificed, and tumors were harvested 6 hours post a single dose. Mice in the Compound 1 10 mg/kg and 25 mg/kg groups were sampled at 6- and 12-hours post a single dose and in the 50 mg/kg group at 4-, 6- and 12-hours post a single dose. Mice in the Osimertinib 25 mg/kg group were sampled at 4-, 6- and 24-hours post a single dose. 3 tumors were collected per each time point sampled. Tumors were then mechanically homogenized, and protein extracted using RIPA buffer (Sigma Aldrich). Protein concentration was quantified using a Pierce™ BCA Protein Assay Kit, samples were reduced, and equal protein amounts were then loaded onto a western blot gel for analysis. Tumors were analyzed for EGFR Receptor L858R Mutant Specific (CST, 3197) or Phospho-EGFR Receptor Tyr1068 (CST, 2234) expression. The intensity of individual bands was measured for data analysis using Image Studio software. Protein expression was quantitated in relation to the reference protein, alpha-tubulin, to control for total protein concentration. The data was then normalized to the amount of target in the Compound 1 treated samples in comparison to the vehicle control samples. Data is represented as percent of target present in the vehicle control and normalized for total protein. Error bars represent ±SEM values.

### Example 57. Efficacy of Compound 1 in Triple Mutant EGFR Model

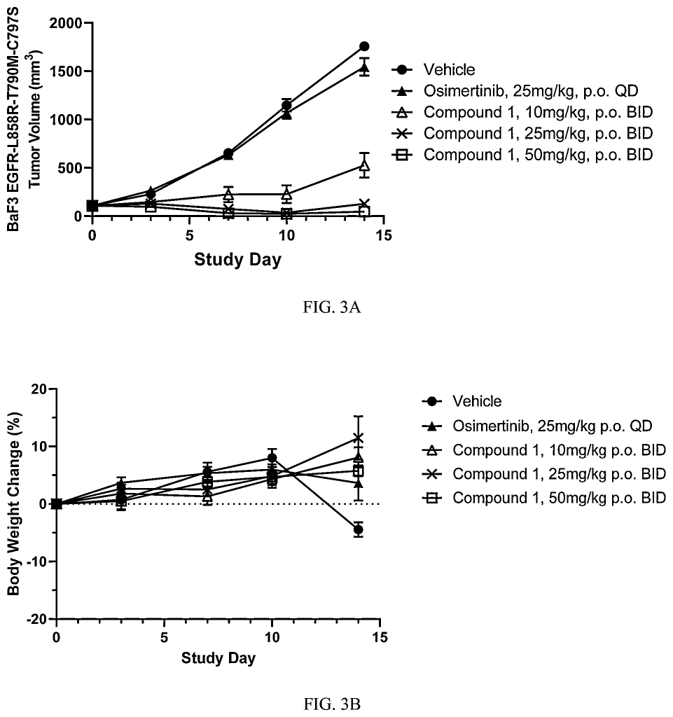

Compound 1 and Compound 2 efficacy in an engineered triple mutant EGFR (L858R-T790M-C797S) BaF3 tumors was evaluated at three different concentrations: 20 mg/kg/day, 50 mg/kg/day, and 100 mg/kg/day. Each Compound was administered BID (twice a day) orally (PO). The efficacy of Compound 1 and Compound 2 was compared to osimertinib (administered orally at 25 mg/kg/day). The results for Compound 1 in the BaF3 tumors are shown in FIG. 3. Table 12 describes the statistical significance of the experiment for each dose. Treatment with Compound 1 at all 20, 50 and 100 mg/kg resulted in a reduced tumor volume.

TABLE 12
Statistical Significance of Compound 1 Compared to Vehicle

| | | |
|---|---|---|
| | | Statistical Significance |
| | Dose Level | Compared to Vehicle |
| Treatment | (mg/kg/day) | (p-value) |

BaF3 Cells

| | | |
|---|---|---|
| Vehicle | N/A | N/A |
| Compound 1 | 20 | <0.0001 |
| Compound 1 | 50 | <0.0001 |
| Compound 1 | 100 | <0.0001 |
| Osimertinib | 25 | >0.05 |

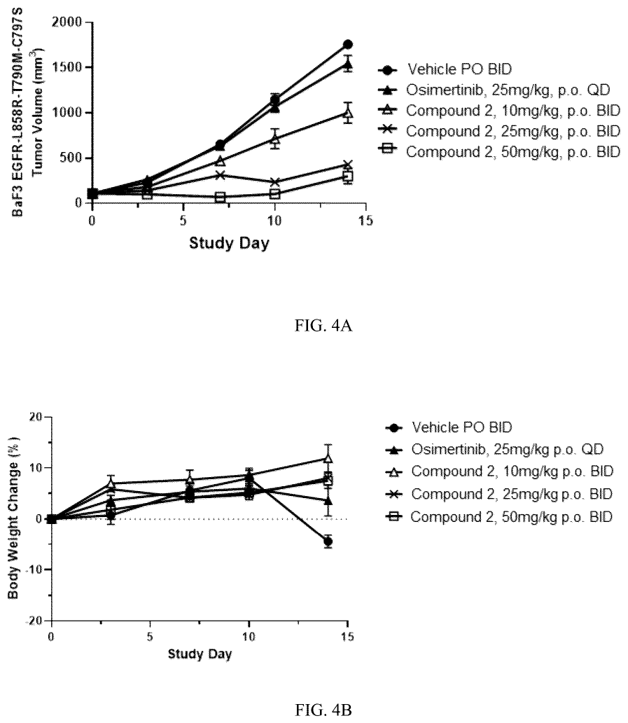

The results of Compound 2 administration in the BaF3 tumors are shown in FIG. 4A and FIG. 4B. Treatment with Compound 2 at all 20, 50 and 100 mg/kg resulted in a reduced tumor volume.

For the BaF3 (EGFR-L858R-T790M-C797S) cells, the allograft study was conducted in female BALB/c nude mice bearing BaF3 tumors. Female nude mice were inoculated subcutaneously in the right flank with 10×10⁶ tumors cells in 0.1 mL of PBS. Tumor volume was measured twice weekly in two dimensions using calipers, and volume (mm³) was calculated using the formula: V=0.5 a×b² where a and b are the long and short diameters of the tumor in mm, respectively. Once the tumors reached an average tumor volume of 108 mm³ (6 days after implantation), the animals were divided randomly into groups of 6, stratified to result in about equal average tumor sizes in each treatment group. Treatment began on Day 0 with established subcutaneous BaF3 tumors with a mean tumor volume (MTV) of 108 mm³.

All agents were administered to mice bearing BaF3 tumors on day 0 and dosed PO daily (osimertinib) or twice a day (Compound 1 and Compound 2) for 14 days. Compound 1 and Compound 2 were dosed at 20, 50, or 100 mg/kg/day and was formulated in PEG400 (20% v/v)+80% v/v SBECD (25% w/v) in ddH2O. Osimertinib was dosed at 25 mg/kg/day and was formulated in 1% Tween 80. Body weight and MTV were measured on a 2× weekly schedule. Statistical analysis was performed using a one-way ANOVA followed by Dunnett's multiple comparison test. Data are expressed as MTV±SEM.

### Example 58. Compound 1 Demonstrates Rapid Tumor Regression in NCI-111975-Luc Intracranial Tumor Model Established by Intracranial Injection of Tumor Cells

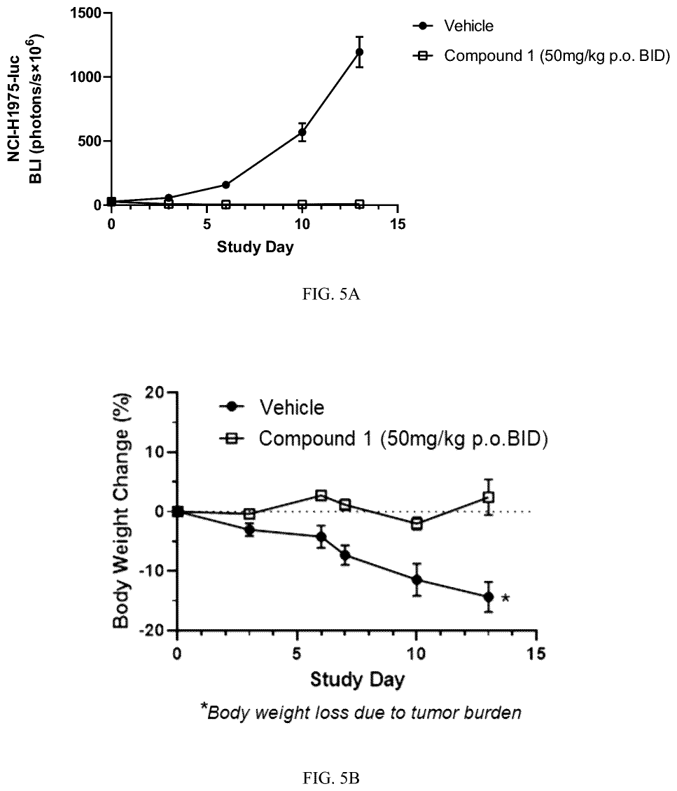

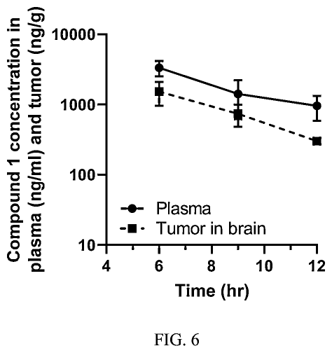

Compound 1 efficacy in a NCI-H1975-luciferase expressing intracranial tumor model established by injecting tumor cells intracranially into the mouse forebrain was evaluated at two different concentrations: 100 mg/kg/day. Compound 1 was administered BID (twice a day) orally (PO). Eight mice were used per group. The results in the intracranial tumor model are shown in FIG. 5A. Table 13 describes the statistical significance of the experiment for each dose. Treatment with Compound 1 at both dose levels 100 mg/kg/day resulted in a reduced tumor volume.

TABLE 13
Statistical Significance of Compound 1 Compared to Vehicle

| | | |
|---|---|---|
| | Dose Level | Statistical Significance |
| Treatment | (mg/kg/day) | Compared to Vehicle |

NCI-H1975-luc Cells

| | | |
|---|---|---|
| Vehicle | N/A | N/A |
| Compound 1 | 100 | 0.001 |

2×10⁵ luciferase-expressing NCI-H1975-luc tumor cells suspended in 2 μL RPMI-1640 media were injected into the right forebrain of female BALB/c nude mice. Tumor growth was monitored using imaging analysis. Mice were injected intraperitoneally with 15 mg/mL of D-luciferin and anesthetized with 1-2% isoflurane inhalation. 10 minutes post luciferin injection, mice were imaged using IVIS Lumina III (Perkin Elmer) and total bioluminescence signal (BLI, photons/s) was measured in a region of interest (ROI). BLI from ROI were quantified and used as an indicator of tumor burden. 7 days post tumor cell inoculation mice were randomized into groups of 8 with a mean BLI of 27×10⁶ photons/s. All agents were administered to mice bearing NCI-H1975-luc brain tumors on day 8. Compound 1 was dosed orally (PO) at 100 mg/kg and was formulated in PEG400 (20% v/v)+80% v/v SBECD (20% w/v) in ddH2O, which was also used as the vehicle control. Compound 1 was administered BID (twice a day). Body weight and MTV were measured on a 2× weekly schedule. Statistical analysis was calculated using the Mann-Whitney U test. Data are expressed as bioluminescent signal (photons/s). The resulting data is shown in FIG. 5A and FIG. 5B.

### Example 59. Ba/F3 EGFR Cell Viability Assays

### Materials

RPMI 1640 media, cell culture flasks, and 384-well microplates were purchased from VWR (Radnor, PA, USA). Fetal bovine serum (FBS) was purchased from Life Technologies (Carlsbad, CA, USA) and CellTiter-Glo® was purchased from Promega (Madison, WI, USA). The Ba/F3 parental cell line was purchased from CrownBio (Santa Clara, CA). BaF3 cell lines ectopically expressing the EGFR mutations L858R, L858R-T790M, L858R-C797S, and L858R-T790M-C797S were purchased from CrownBio and EGFR mutations L858R-L718Q, L858R-T790M-L718Q, L858R-L792H, and L858R-T790M-L792H, were purchased from Signosis (Santa Clara, CA).

### Methods

Cell viability was assessed based on the quantification of ATP using the CellTiter-Glo® assay (Promega, Madison, WI, USA). To determine the effect of compounds on cell viability, all Ba/F3 cell lines were plated at a cell density of 2000 cells/well in 384-well plates using RPMI 1640+10% FBS and treated with compounds of interest using a 10-point half-log dilution series in duplicate for 72 hours, with the highest dose set at 10 mM. CellTiter-Glo® reagent was then added and luminescence signals were measured using an EnVision™ Multimode plate reader (Perkin Elmer, Santa Clara, CA). For data normalization, cells untreated with the test compounds at Time=72 hours were set to 100% (equivalent to maximal cell growth after 72 hours); cells untreated with the test compounds at Time=0 hours were set to 0% (equivalent to cytostasis); and media-only wells were set to −100% (equivalent to complete cytotoxicity). % Viability was determined by normalizing the signal with positive and DMSO treated negative controls on the same microtiter plate. The half-maximal inhibition of cell growth (GI50) is computed from where the fitted curve crosses 50% while the half-maximal lethal dose (LD50) is computed from where the fitted curve crosses −50%.

### Example 60. Kinome Selectivity Assay Experimental

Kinome-wide binding selectivity of CFT8919 was assessed via the KINOMEscan scanMAX assay at Eurofins DiscoverX (Freemont, CA, USA). Compound was tested at 100 nM against a diverse panel of 468 kinases. The size of circles was mapped onto the kinase phylogenetic tree utilizing DiscoverX TREEspot using <50% of % control as a cutoff.

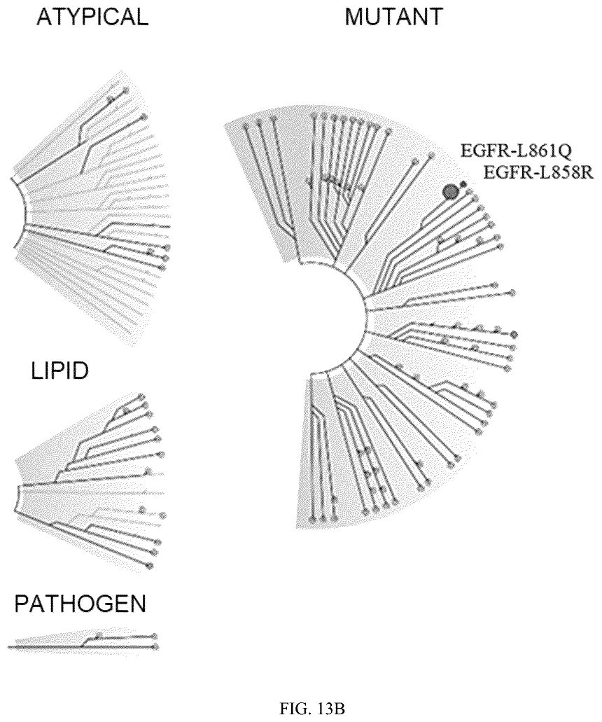

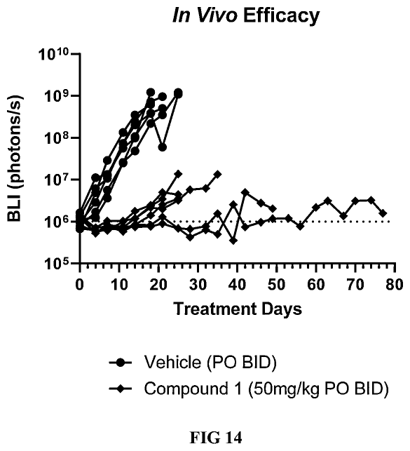

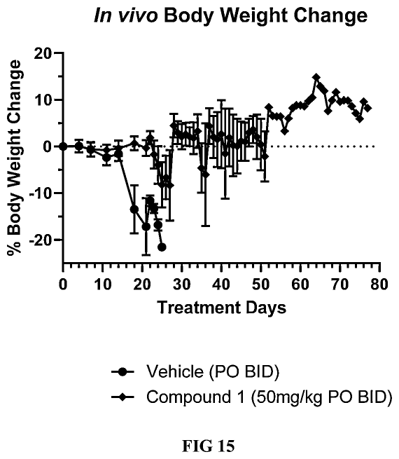

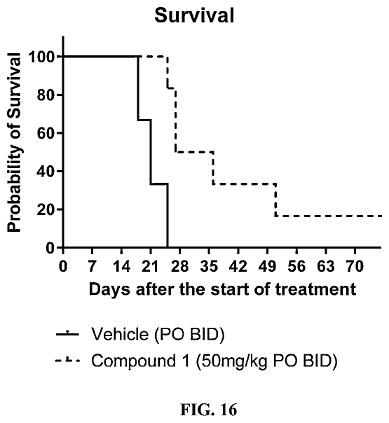

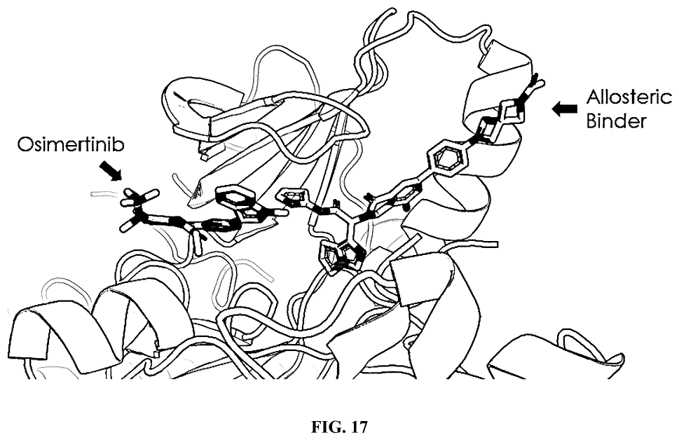

For most assays, kinase-tagged T7 phage strains were grown in parallel in 24-well blocks in an E. coli host derived from the BL21 strain. E. coli were grown to log-phase and infected with T7 phage from a frozen stock (multiplicity of infection=0.4) and incubated with shaking at 32° C. until lysis (90-150 minutes). The lysates were centrifuged (6,000×g) and filtered (0.2 μm) to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce non-specific phage binding. Binding reactions were assembled by combining kinases, liganded affinity beads, and test compounds in 1× binding buffer (20% SeaBlock, 0.17×PBS, 0.05% Tween 20, 6 mM DTT). Test compounds were prepared as 40× stocks in 100% DMSO and directly diluted into the assay. All reactions were performed in polypropylene 384-well plates in a final volume of 0.02 ml. The assay plates were incubated at room temperature with shaking for 1 hour and the affinity beads were washed with wash buffer (lx PBS, 0.05% Tween 20). The beads were then re-suspended in elution buffer (lx PBS, 0.05% Tween 20, 0.5 μM non-biotinylated affinity ligand) and incubated at room temperature with shaking for 30 minutes. The kinase concentration in the eluates was measured by qPCR. The resulting data is shown in FIG. 13A and FIG. 13B.

### Example 61 Global Proteomic Experiments for Compound 1 in A431 and NCI-H1975 Cell Lines

Each cell line was treated in duplicate with either DMSO or Compound 1 (final concentration 300 nM) for 6 hours in an incubator at 37° C. with 5% CO₂. After 6 hour incubation, cells were harvested, washed twice with PBS, and snap frozen in liquid nitrogen. Samples were re-suspended in lysis buffer [8 M urea, 50 mM HEPS (pH 8.5), 50 mM NaCl, 1× protease inhibitor cocktail] and lysed by sonication at 4° C. with 85% amplitude in a pulse setting with 30 seconds on and 30 seconds off for a total sonication time of 5 minutes. Lysates were centrifuged at maximum speed for 10 minutes at 4° C. and the supernatant collected, reduced for 1 hour at room temperature with 5 mM TCEP, and the cysteine residues were then alkylated with 15 mM iodoacetamide (room temperature, in dark, 30 minutes). Protein content was extracted by methanol-chloroform precipitation and subsequent ice-cold acetone washes. Protein pellets were resuspended in 8 M urea, 50 mM HEPES (pH 8.5) buffer and protein concentrations were measured by BCA assay. Samples were then diluted to 4 M urea with 50 mM HEPES (pH 8.5) and digested at 37° C. for 1 hour with endoproteinase Lys-C, at a 1/100 enzyme/protein ratio. The mixtures were then diluted to 1 M urea with 50 mM HEPES (pH 8.5) and trypsin was added at a 1/100 enzyme/protein ratio. The reaction was incubated overnight at 37° C. and stopped by acidification with formic acid to a final concentration of 5% (v/v). Peptides were purified using C18 SepPak solid-phase extraction cartridges and dried to completion using a SpeedVac system.

For peptide tandem mass tag (TMT) labeling, 100 μg of peptides per sample was prepared at 1 μg/μl concentration in 200 mM HEPES (pH 8.5), 30% acetonitrile (ACN) and the specific TMT reagent. After 1 hour incubation at room temperature, reactions were quenched with 0.3% hydroxylamine for 15 minutes and mixed equally. The mixed sample was desalted using C18 SepPak solid-phase extraction cartridges, and dried to completion in a SpeedVac. Dried peptides were resuspended in 5% ACN, 10 mM NH₄HCO₃ pH 8 and fractionated in a basic pH reversed phase chromatography using a HPLC equipped with a 3.5 μm)(Bridge Peptide BEH C18 column. 96 fractions were collected, which were consolidated into 12 samples, desalted using SepPAK C18 cartridges, and then dried via vacuum centrifugation. Samples were then reconstituted in 164, of 5% formic for LC-MS/MS/MS analysis.

6 μL of each sample was separated by reversed phase chromatography using an EASY-Spray C18 column (2 μm particle size, 500 mm length×75 μm ID) mounted in an EASY-nLC 1200 LC pump coupled to an Orbitrap Fusion Lumos Tribid mass spectrometer. Peptides were separated using a 450 min gradient divided into 3 sections (5 to 25% ACN for 300 minutes, 25-40% ACN for 120 minutes, and 95% ACN for 30 minutes) at a flow rate of 300 nL/min. Peptides were collected in a data dependent method using CID for MS2 fragmentation and HCD for synchronous precursor selection based MS3 (SPS-MS3) fragmentation for TMT reporter ion release. All data files obtained from the mass spectrometer were processed using SEQUEST-based software developed by Professor Steve Gygi's laboratory at Harvard Medical School. Briefly, mass spectra were searched against the human non-redundant Uniprot protein database concatenated with a database composed of all protein sequences in the reversed order as well as known contaminants. In all SEQUEST searches, precursor ion tolerance was set at 25 ppm, including methionine oxidation (+15.9949 Da) and cysteine carbamidomethylation (+57.0215 Da) as variable modifications. TMT tag (+229.1629 Da) on lysine residues and peptide N-termini were set as static modifications. Peptide-spectrum matches (PSMs) were performed using a linear discriminant analysis and adjusted to a 2% false discovery rate (FDR). TMT reporter ion intensities were quantified by extracting the signal-to-noise ratio for each. Peptides were next collapsed into protein groups using a 4% FDR target. The resulting data is summarized below:

TABLE 14
Proteome-Wide Degradation Selectivity

| | | # of | # of proteins with |
|---|---|---|---|
| Cell | EGFR | proteins | >50% protein level |
| Line | Genotype | detected | decrease* |
| A431 | Wild-type | 9190 | 0 |
| H1975 | L858R-T790M | 8853 | 2 [EGFR, CCND1⁺] |

*p-value < 0.001
⁺Likely due to the biological effect of EGFR suppression; similar change observed upon Osimertinib treatment

### Example 62 SALL4 Degradation

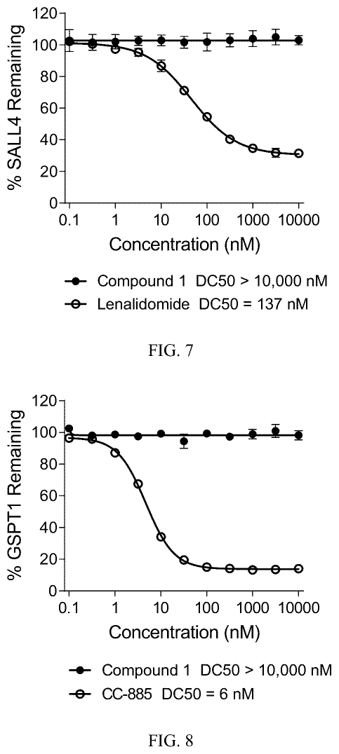

Specific studies were carried out to assess the ability of Compound 1 to degrade Sal-like protein 4 (SALL4), a protein implicated in the teratogenicity induced by the IMiD®s class of IKZF1/IKZF3 degraders including lenalidomide (Matyskiela et al., Crystal structure of the SALL4-pomalidomide-cereblon-DDB1 complex, Nature Structural and Molecular Biology, (2020) 27, 319-322). Endogenous expression of SALL4 in KELLY cells was determined by addition of C-terminal HiBiT tag to the KELLY cell line (DSMZ ACC355) using CRISPR-Cas9. Briefly, test compound was added to 384-well plates at a top concentration of 1 μM with 11 points, half log titration in duplicates. KELLY cells expressing HiBiT-tagged SALL4 were seeded into the 384-well plates in RPMI medium containing 10% FBS and 2 mM pyruvate at a cell density of 6000 cells per well. Cells treated in the absence of the test compound were the negative control and wells containing media only were the positive control. Following compound treatment, cells were incubated at 37° C. with 5% CO2 for 6 hr. HiBiT signal was determined using Nano-Glo™ HiBiT Lytic Assay System (Promega, N3050) and luminescence was acquired on EnVision™ Multilabel Reader (PerkinElmer, Santa Clara, CA, USA). % SALL4 remaining was determined by normalizing the signal with positive and negative controls on the same microtiter plate. Data in FIG. 7 are expressed as the mean±SD. Curve fit and IC₅₀ determination was performed by 4 parametric regression analysis.

### Example 63 GSPT1 Degradation

Some cereblon E3 ligase modulators have been shown to promote degradation of the GI to S phase transition 1, GSPT1 with potential for phenotypically relevant off-target effects. Endogenous expression of GSPT1 in 293T cells was determined by addition of N-terminal HiBiT tag to the 293T cell line (ATCC CRL-3216) using CRISPR-Cas9. Briefly, test compound was added to 384-well plates at a top concentration of 10 μM with 11 points, half log titration in duplicates. 293T cells expressing HiBiT-tagged GSPT1 were seeded into the 384-well plates in DMEM medium containing 10% FBS at a cell density of 6000 cells per well. Cells treated in the absence of the test compound were the negative control and wells containing media only were the positive control. Following compound treatment, cells were incubated at 37° C. with 5% CO2 for 6 hr. HiBiT signal was determined using Nano-Glo™ HiBiT Lytic Assay System (Promega, N3050) and luminescence was acquired on EnVision™ Multilabel Reader (PerkinElmer, Santa Clara, CA, USA). % GSPT1 remaining was determined by normalizing the signal with positive and negative controls on the same microtiter plate. Data in FIG. 8 are expressed as the mean±SD. Curve fit and IC50 determination was performed by 4 parametric regression analysis.

### Example 64 Measurement of Body Weight, Bioluminescence Signal, and Survivability in NCI-111975-Luc Intracranial Tumor Model Established by Intracaroid Artery Injection of Tumor Cells with Compound 1 Treatment

Female BALB/c nude mice were inoculated by intracarotid injection with NCI-H1975-luc cells (1×10⁵ in 100 μL PBS) for tumor development. Tumor growth was monitored using imaging analysis. Tumor-bearing mice were weighed and injected intraperitoneally with luciferin at a dose of 150 mg/kg and anesthetized with a mixture gas of oxygen and isoflurane. 10 minutes post luciferin injection, mice were imaged using IVIS Lumina III machine and total bioluminescence signal (BLI, photons/s) was measured in a region of interest (ROI). BLI from ROI were quantified and used as an indicator of tumor burden. Twenty days post tumor cell inoculation mice were randomized into groups of 6 with a mean BLI of 1.08×10⁶ photons/s. Compound 1 was dosed orally (PO) twice a day at 50 mg/kg and was formulated in 20% PEG400+80% (25% SBECD, “sulfobutylether-β-cyclodextrin”) in double distilled H₂Om which was also used as the vehicle control. Body weight and MTV were measured on a 2× weekly schedule. Survival data was also measured and is represented as median survival time±SEM.

### Example 65 EGFR-L858R Protein Expression and Purification Used in Example 66 Co-Crystallization Study

The mutant 2×StrepII-TEV-EGFR (L858R/V948R) protein, residues 696-1022, was expressed in Sf9 insect cells. After two days of expression the 4.8 L of Sf9 cells were harvested and then resuspended in lysis buffer (50 mM Tris-HCl pH 7.5, 500 mM NaCl, 10% glycerol, 1 mM TCEP, 1 mM PMSF) mixed with 1 mM MgCl₂, nuclease, and cOmplete Protease Inhibitor Tablet (Roche). The lysate was homogenized, lysed, and centrifuged for 60 min at 16,000 rpm while being kept at 4° C. The supernatant was incubated with Strep-Tactin®XT beads and washed extensively with Buffer A (50 mM Tris-HCl pH 7.5, 500 mM NaCl, 10% glycerol, 1 mM TCEP, and 1 mM PMSF). The 2×StrepII-TEV-EGFR (L858R/V948R) protein was eluted with Buffer A plus 75 mM biotin. The pooled elution fractions were incubated with His-TEV protease overnight where complete digestion was observed. The mixture was subjected to reverse Ni-affinity by loading onto a HisTrap Excel (Cytiva) Ni Sepharose column to remove the His-TEV protease. The column was washed with Buffer A and the flow through was collected. The cleaved material was concentrated and loaded onto a HiLoad 16/600 Superdex 200 pg size exclusion column with SEC buffer (25 mM Tris-HCl pH 8.0, 100 mM NaCl, 10% glycerol, and 1 mM TCEP). A singular peak from the chromatogram was observed. Fractions from this peak were pooled and the resulting purified protein was concentrated to 6.3 mg/mL and flash frozen at −80° C.

### Example 66 Cocrystallization of EGFR-L858R Protein with the EGFR Binding Portion of Compound 1 and Osimertinib

The concentrated EGFR (L858R/V948R) (6.3 mg/mL) was incubated with two-fold excess of osimertinib followed by 1.5-fold excess of the allosteric EGFR binding portion of Compound 1 for 60 min on ice. Crystals were grown using the sitting drop vapor diffusion method at 20° C. by combining 100 nL of protein-small molecule solution with 50 nL of reservoir solution containing 100 mM MES pH 6.5 and 25% PEG MME 2K. After 8 days the crystals were harvested with 20% ethylene glycol as cryoprotectant and flash frozen in liquid nitrogen.

Data were collected from a single crystal at NSLSii FMX beamline 17-ID-2 and processed using DIALS. See Winter, G.; Waterman, D. G.; Parkhurst, J. M.; Brewster, A. S.; Gildea, R. J.; Gerstel, M.; Fuentes-Montero, L.; Vollmar, M.; Michels-Clark, T.; Young, I. D.; Sauter, N. K.; Evans, G. DIALS: Implementation and Evaluation of a New Integration Package. Acta Crystallogr Sect D Struct Biology 2018, 74 (2), 85-97. The protein complex structure was solved using PhaserMR in the CCP4 suite with the molecular replacement search model PDB ID: 7JXP. For PhaserMR methods see McCoy, A. J.; Grosse-Kunstleve, R. W.; Adams, P. D.; Winn, M. D.; Storoni, L. C.; Read, R. J. Phaser Crystallographic Software. J Appl Crystallogr 2007, 40 (4), 658-674. Structures were refined using iterative rounds of model building in Coot followed by refinement using Refmac5. For model building in Coot methods see Emsley, P.; Cowtan, K. Coot: Model-Building Tools for Molecular Graphics. Acta Crystallogr Sect D Biological Crystallogr 2004, 60 (12), 2126-2132. For Refmac5 methods see Winn, M. D.; Ballard, C. C.; Cowtan, K. D.; Dodson, E. J.; Emsley, P.; Evans, P. R.; Keegan, R. M.; Krissinel, E. B.; Leslie, A. G. W.; McCoy, A.; McNicholas, S. J.; Murshudov, G. N.; Pannu, N. S.; Potterton, E. A.; Powell, H. R.; Read, R. J.; Vagin, A.; Wilson, K. S. Overview of the CCP4 Suite and Current Developments. Acta Crystallogr Sect D Biological Crystallogr 2011, 67 (4), 235-242. For search model see PDB ID: 7JXP see Beyett, T. S.; To, C.; Heppner, D. E.; Rana, J. K.; Schmoker, A. M.; Jang, J.; Clercq, D. J. H. D.; Gomez, G.; Scott, D. A.; Gray, N. S.; Janne, P. A.; Eck, M. J. Molecular Basis for Cooperative Binding and Synergy of ATP-Site and Allosteric EGFR Inhibitors. Nat Commun 2022, 13 (1), 2530.”

The EGFR binding portion of Compound 1 used for this experiment is structure:

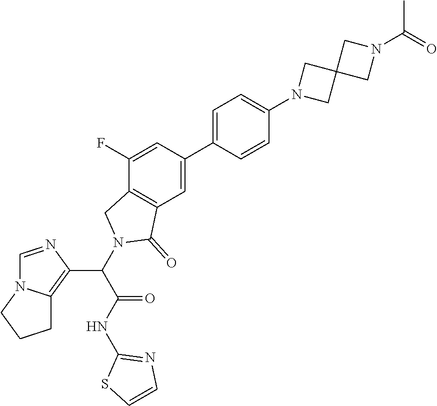 [SMILES: CC(=O)N1CC2(C1)CN(c1ccc(-c3cc(F)c4c(c3)C(=O)N(C(C(=O)Nc3nccs3)c3ncn5c3CCC5)C4)cc1)C2]

### Example 67 Differential Scanning Fluorimetry (DSF)

The thermal stability of EGFR (L858R) was measured using differential scanning fluorimetry. The protein was incubated at 10 μM in buffer (100 mM HEPES pH 7.5 and 150 mM NaCl) with DMSO to determine the stability of the protein alone. In addition, the protein (10 μM) was incubated with each compound (100 μM) individually, or in combination, for 30 min on ice. Protein stability was measured in the presence of 4×SYPRO™ Orange dye (Thermo Fisher) using a ViiA7 Real-Time PCR System (Thermo Fisher) adapted for thermal melts. Reactions of 20 μL were run in quadruplicate using Microamp Optical 384-well reaction plates (Applied Biosystems). Temperature was increased by 0.5° C. intervals from 25° C. to 99° C. and fluorescence intensity was measured. In-house curve fitting analysis software was used to calculate the thermal melting point (T_{M}) of the protein in each reaction.

| | ΔT_{M} |
|---|---|
| | (° C.) |
| DMSO | — |
| Compound 1 | 5.8 |
| osimertinib | 10.6 |
| osimertinib + Compound 1 | 14.6 |

DSF Table: The change in thermal stability compared to the apo proten (+DMSO; T_{M} = 45.4° C.) was calculated and reported as ΔT_{M} (° C). The degrader Compound 1 and Osimertinib alone showed stabilization effects of 5.8° C. and 10.6° C. respectively. However, in combination their effect was additive yielding a ΔT_{M} = 14.6° C., suggesting they can both engage EGFR simultaneously.

### Example 68 SPR Assay to Assess EGFR^{L858R}: Compound 1: CRBN-DDB1 Ternary Complex Formation in the Presence and Absence of Osimertinib

### Proteins & Reagents

CRBN (residues 1-442 with an N-terminal Avi-tag) in complex with DDB 1 (residues 4-1143) (CRBN-DDB 1) and EGFR^{L858R} (residues 696-1022) proteins were expressed recombinantly in High Five (Thermo Fisher Scientific) and Sf9 insect cells, respectively, and subsequently purified. The CRBN-DDB1 protein complex was then biotinylated (Btn-CRBN-DDB1). Stock solutions for osimertinib and Compound 1 were prepared at 10 mM in DMSO. 100× working stocks of Compound 1 were then prepared at 1 μM, 3.16 μM, 10 μM, 31.6 μM, and 100 μM by dilution with DMSO.

### SPR Experiments

All SPR experiments were conducted using a Bruker Sierra SPR-32 Pro instrument with a Biotin-Tag Capture (BTC) sensor chip equilibrated at 25° C. in Running Buffer (50 mM HEPES pH 7.4, 200 mM NaCl, 0.5 mM TCEP, 0.05% Tween-20, 1% DMSO). The flow rate for all steps is 30 μL/min unless otherwise specified. Btn-CRBN-DDB1 protein was diluted to 150 μg/mL in Running Buffer and injected over two sensor spots of a pre-conditioned BTC sensor for 60 s at a flow rate of 7.5 μL/min, achieving final responses of 3240 RU and 2455 RU for experiments conducted with Compound 1 plus EGFR^{L858R} in the apo form (EGFRₐₚₒ^{L858R}) or osimertinib bound form (EGFR_{osimertinib}^{L858R}), respectively. Sensor surfaces were then equilibrated with five 300 second injections of Running Buffer.

Analyte samples were prepared with a variable concentration of Compound 1 (or an equivalent volume of DMSO for referencing) and a fixed concentration of either EGFRₐₚₒ^{L858R} or EGFR_{osimertinib}^{L858R} as detailed below.

EGFRₐₚₒ^{L858R} protein was first desalted into Sample Loading Buffer (50 mM HEPES pH 7.4, 200 mM NaCl, 0.5 mM TCEP, 0.05% Tween-20) using a 0.5 mL Zeba spin desalting column (Thermo Fischer Scientific) in accordance with the manufacturer's instructions. Protein concentration was then measured by absorbance at 280 nm (ε₂₈₀=52370 M⁻¹cm^{-b 1}). EGFRₐₚₒ^{L858R}+Compound 1 analyte samples were prepared by first diluting desalted EGFRₐₚₒ^{L858R} with Sample Loading Buffer to a 1.52 μM working stock which was then mixed in a 1:99 (Compound 1: EGFRₐₚₒ^{L858R}) volume ratio with one of the 100× Compound 1 working stocks listed above (or with an equivalent volume of DMSO) to generate six samples with a final EGFRₐₚₒ^{L858R} concentration of 1.511M and final Compound 1 concentrations of 0 nM, 10 nM, 31.6 nM, 100 nM, 316 nM or 1000 nM.

To prepare EGFR_{osimertinib}^{L858R}+Compound 1 samples, desalted EGFRₐₚₒ^{L858R} and osimertinib were first diluted to respective concentrations of 5.05 μM and 10.1 μM in Sample Loading Buffer and incubated for 15 minutes at room temperature to produce EGFR_{osimertinib}^{L858R}. EGFR_{osimertinib}^{L858R} was then mixed in a 1:99 (Compound 1: EGFR_{osimertinib}^{L858R}) volume ratio with one of the 100× Compound 1 working stocks listed above (or with an equivalent volume of DMSO) to generate six samples with a final EGFR_{osimertinib}^{L858R} concentration of 5.011M and final Compound 1 concentrations of 0 nM, nM, 31.6 nM, 100 nM, 316 nM or 1000 nM.

Analyte samples were injected in increasing order of Compound 1 concentration with each injection cycle comprised of a 160 s injection period followed by a dissociation period of 320 seconds. To account for bulk solvent effects resulting from excess DMSO present in the EGFR_{osimertinib}^{L858R}+Compound 1 samples as a consequence of osimertinib addition, five 30 second solvent correction injections were conducted by injecting Sample Loading Buffer containing 0.9%, 1.0%, 1.1%, 1.2%, or 1.3% DMSO.

### Data Analysis

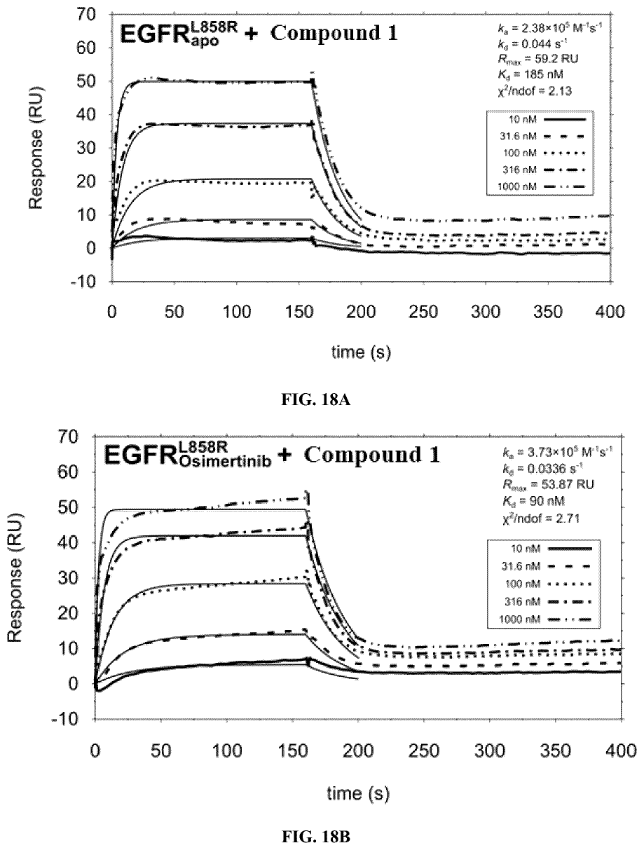

All SPR experiments were processed and analyzed using Sierra Analyzer 3.4.5. Analyte sensorgrams were double referenced to both a BTC sensor spot without immobilized Btn-CRBN-DDB1 as well as a reference injection with 0 nM Compound 1. EGFR_{osimertinib}^{L858R}+Compound 1 sensorgrams were also solvent corrected using the software's solvent correction feature and the five DMSO solvent correction injections described above. The built-in 1:1 Langmuir binding model was then used to globally fit SPR sensorgrams for all Compound 1 concentrations in either EGFRₐₒₚ^{L585R}+Compound 1 or EGFR_{osimertinib}^{L858R}+Compound 1 titration experiments. A local parameter (Rₘᵢₙ) was floated to account for residual bulk shifts during the injection period. Due to baseline drift and subsequent evolution of a baseline offset upon consecutive analyte injections, only the initial 40 s of the dissociation period were included in data fitting. The resulting data is shown in FIGS. 18A and 18B.

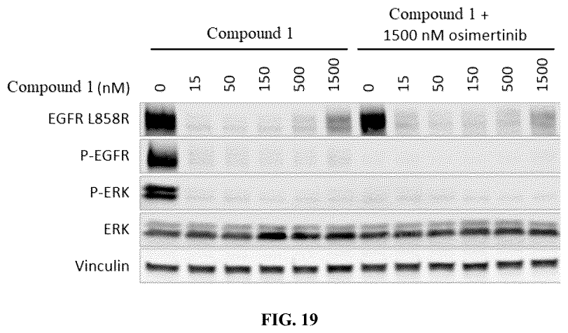

Example 69 Western Blot Experiment on the Combination of Compound 1 and Osimertinib NCI-H3255 cell lines were prepared and then incubated for six hours with 0, 15, 50, 150, 500, or 1,500 nM of Compound 1 in the presence or absence of 1,500 nM osimertinib. The compound bearing solutions were formulated to achieve a 0.1% final concentration of DMSO. As shown in the western blot image (FIG. 19) Compound 1 efficiently degraded EGFR-L858R and inhibited phospho-EGFR and phospho-ERK in the presence and absence of osimertinib. Antibodies used in western blot were purchased from the following vendors: EGFR-L858R specific antibody (CST 3197), pY1068 EGFR (CST 2234), pERK (CST 4370), ERK (CST 4695), vinculin (Millipore 05-386).

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teaching of this invention that certain changes and modifications may be made thereto without departing from the spirit or scope of the invention as defined in the appended claims. Additionally, those skilled in the art will recognize, or be able to ascertain using no more than routine experimentation, many equivalents to the specific embodiments and methods described herein. Such equivalents are intended to be encompassed by the scope of the present application.

We claim:

1. A method of treating a brain or central nervous system metastasis of an EGFR mediated cancer in a human patient comprising administering an effective amount of a compound of formula: 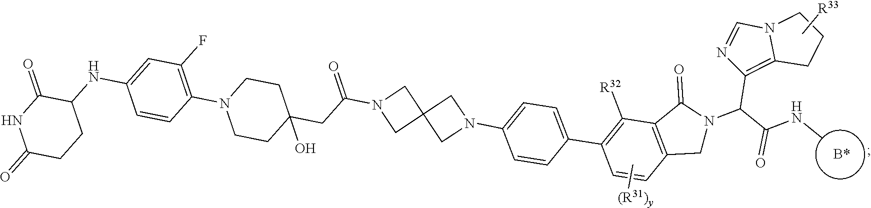
or a pharmaceutically acceptable salt thereof to a human patent in need thereof,
wherein:
B* is heteroaryl or aryl, each of which is optionally substituted with 1, 2, or 3 R³¹ substituents;
y is 0 or 1;
R³¹ is independently selected at each occurrence from the group consisting of F, Cl, Br, C₁₋₆-alkyl, cyano, C₁₋₆-alkoxy, halo-C₁₋₆-alkoxy, C₃₋₈-cycloalkyl, and halo-C₃₋₈-cycloalkyl and can be located on either ring when present on a bicycle;
R³² is hydrogen, F, Cl, Br, C₁₋₆-alkyl, C₃₋₈-cycloalkyl, or halo-C₃₋₈-cycloalkyl; and
R³³ is hydrogen, F, Cl, Br, C₁₋₆-alkyl, C₃₋₈-cycloalkyl, or halo-C₃₋₈-cycloalkyl and can be located on the dihydropyrrole or imidazole ring.

2. The method of claim 1, wherein B* is

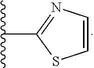 [SMILES: *c1nccs1]

3. The method of claim 2, wherein y is 1.

4. The method of claim 3, wherein R³¹ is selected from the group consisting of F, C₁₋₆-alkyl, cyano, C₁₋₆-alkoxy, and halo-C₁₋₆-alkyl.

5. The method of claim 3, wherein R³¹ is F.

6. The method of claim 5, wherein R³² is hydrogen.

7. The method of claim 6, wherein R³³ is hydrogen.

8. The method of claim 1, wherein B* is

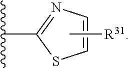

9. The method of claim 8, wherein R³¹ is selected from the group consisting of F, C₁₋₆-alkyl, cyano, C₁₋₆-alkoxy, and halo-C₁₋₆-alkyl.

10. The method of claim 9, wherein R³² is hydrogen.

11. The method of claim 10, wherein R³³ is hydrogen.

12. The method of claim 1, wherein the compound is of structure: 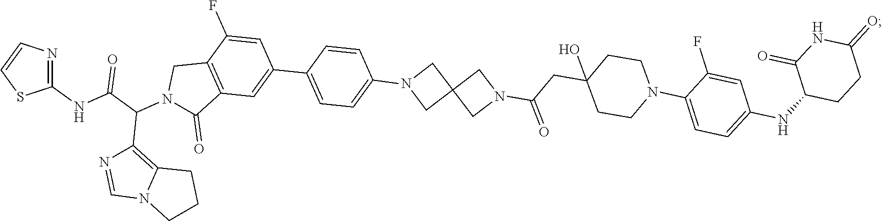 [SMILES: O=C1CC[C@H](Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1]
or pharmaceutically acceptable salt thereof.

13. The method of claim 1, wherein the compound is of structure: 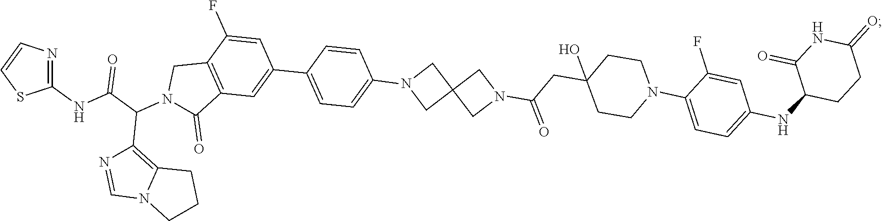 [SMILES: O=C1CC[C@@H](Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1]
or pharmaceutically acceptable salt thereof.

14. The method of claim 1, wherein the compound is of structure: 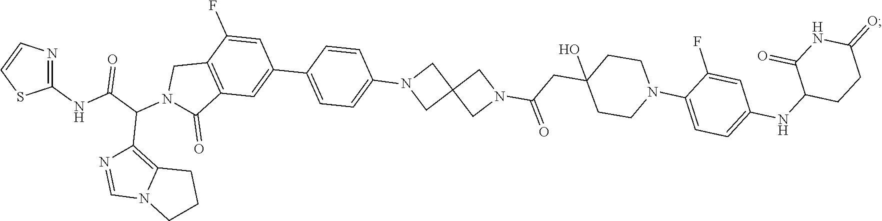 [SMILES: O=C1CCC(Nc2ccc(N3CCC(O)(CC(=O)N4CC5(C4)CN(c4ccc(-c6cc(F)c7c(c6)C(=O)N(C(C(=O)Nc6nccs6)c6ncn8c6CCC8)C7)cc4)C5)CC3)c(F)c2)C(=O)N1]
or pharmaceutically acceptable salt thereof.

15. The method of claim 1, wherein the EGFR mediated cancer is a lung cancer.

16. The method of claim 15, wherein the lung cancer is non-small cell lung cancer.

17. The method of claim 15, wherein the lung cancer is small cell lung cancer.

18. The method of claim 1, wherein the EGFR mediated cancer is adenocarcinoma.

19. The method of claim 1, wherein the EGFR mediated cancer is selected from the group consisting of squamous cell lung cancer, large-cell undifferentiated carcinoma, neuroendocrine carcinoma, sarcomatoid carcinoma, adenosquamous carcinoma, oat-cell cancer, combined small cell carcinoma, lung carcinoid tumor, central carcinoid, peripheral carcinoid, salivary gland-type lung carcinoma, mesothelioma, mediastinal tumor, colorectal cancer, rectal cancer, head and neck cancer, esophageal cancer, pancreatic cancer, thyroid cancer, ovarian cancer, uterine cancer, cervical cancer, kidney cancer, liver cancer, bladder cancer, and melanoma.

20. The method of claim 1, wherein the EGFR mediated cancer is breast cancer.

21. The method of claim 20, wherein the breast cancer is HER-2 positive breast cancer.

22. The method of claim 20, wherein the breast cancer is estrogen receptor positive breast cancer.

23. The method of claim 20, wherein the breast cancer is progesterone receptor positive breast cancer.

24. The method of claim 20, wherein the breast cancer is triple negative breast cancer.

25. The method of claim 1, wherein the EGFR mediated cancer is relapsed and/or refractory.

26. The method of claim 1, wherein the compound is administered orally.

27. The method of claim 26, wherein the compound is administered orally by a tablet administration.

28. The method of claim 26, wherein the compound is administered orally by a capsule administration.

29. The method of claim 1, wherein the compound is administered parenterally.

30. The method of claim 29, wherein the compound is administered intravenously.
